(19) United States
(12) Patent Application Publication
(10) Pub. No.: US 2024/0116921 A1
(43) Pub. Date: Apr. 11, 2024

(54) LACTAM (HETERO)ARYLFUSEDPYRIMIDINE DERIVATIVES AS INHIBITORS OF ERBB2

(71) Applicant: EnLiven Therapeutics, Inc., Boulder, CO (US)

(72) Inventors: Joseph P. LYSSIKATOS, Boulder, CO (US); Samuel KINTZ, Boulder, CO (US); Li REN, Superior, CO (US)

(21) Appl. No.: 18/258,765

(22) PCT Filed: Dec. 21, 2021
(86) PCT No.: PCT/US2021/073047

§ 371 Date: Jun. 21, 2023

Related U.S. Application Data
(60) Provisional application No. 63/263,072, filed on Oct. 26, 2021.
(60) Provisional application No. 63/226,619, filed on Jul. 28, 2021.
(60) Provisional application No. 63/129,309, filed on Dec. 22, 2020.

(51) Int. Cl. C07D 471/04; C07D 401/14; C07D 403/14; C07D 513/04; C07D 519/00

(52) U.S. Cl. CPC C07D 471/04; C07D 401/14; C07D 403/14; C07D 513/04; C07D 519/00

(57) ABSTRACT

The present disclosure relates generally to compounds and compositions thereof for inhibition of ErbB2, including mutant forms of ErbB2, particularly those harboring an Exon 20 mutation, methods of preparing said compounds and compositions, and their use in the treatment or prophylaxis of various cancers, such as lung, glioma, skin, head neck, salivary gland, breast, esophageal, liver, stomach (gastric), uterine, cervical, biliary tract, pancreatic, colorectal, renal, bladder or prostate cancer.

### CROSS-REFERENCE TO RELATED APPLICATIONS

This application claims priority to and the benefit of U.S. Provisional Application No. 63/129,309, filed on Dec. 22, 2020, U.S. Provisional Application No. 63/226,619, filed on Jul. 28, 2021, and U.S. Provisional Application No. 63/263,072, filed on Oct. 26, 2021, the disclosures of each of which are incorporated herein by reference in their entireties.

### FIELD OF THE INVENTION

The present disclosure relates generally to compounds and compositions thereof for inhibition of ErbB2, including mutant forms of ErbB2, particularly those harboring an Exon 20 mutation, methods of preparing said compounds and compositions, and their use in the treatment or prophylaxis of various cancers, such as lung, glioma, skin, head and neck, salivary gland, breast, esophageal, liver, stomach (gastric), uterine, cervical, biliary tract, pancreatic, colorectal, renal, bladder or prostate cancer.

### BACKGROUND

ErbB2 (or HER2) is a member of the ErbB receptor tyrosine kinase family consisting of four related receptors, including ErbB1 (also known as epidermal growth factor receptor, or EGFR), ErbB3 and ErbB4. Although there are no known ligands that bind to monomeric ErbB2, it can dimerize with other ErbB receptors, particularly ErbB3, and regulate downstream signaling cascades including, but not limited to, the MAPK and PI3K pathways, that promote cell proliferation and survival. Aberrant overexpression of ErbB2 or certain genetic alterations (including point mutations that lead to certain amino acid substitutions or small in-frame insertions in Exon 20 that lead to the deletion and/or insertion of certain small stretches of amino acids) are known to confer elevated or constitutive tyrosine kinase activation to the receptor. Accordingly, the overexpression or mutation of ErbB2 is highly associated with aggressive forms of solid cancers, including breast, ovarian, stomach, and lung cancer (NSCLC).

Currently, there are few approved treatments for cancers associated with ErbB2 overexpression, including tyrosine kinase inhibitors (TKIs) such as tucatinib. Although these TKIs can be effective at ameliorating cancers associated with ErbB2 overexpression, their therapeutic utility is often limited by inadequate selectivity for ErbB2 over EGFR, and consequently are dose-limited by toxicity concerns related to EGFR inhibition (especially gastrointestinal and skin-related toxicities). These toxicities necessitate restrictive dosing regimens, leading to suboptimal target engagement and thus limited therapeutic benefit. Moreover, while current TKIs provide therapeutic benefit for cancers driven by ErbB2 overexpression, they may have limited efficacy in patients harboring specific genetic alterations, such as EGFR or ERBB2 exon 20 insertions, specific point mutations or genetic alterations associated with ErbB family ligands, such as NRG1 gene fusions.

For example, in a small proportion of lung cancer patients, certain especially pernicious mutations in EGFR and ErbB2 known as EGFR exon 20 insertions/ErbB2 insertions are markedly less sensitive to first and second generation reversible TKIs. An added challenge to the development of viable therapies for these specific ErbB Exon 20 mutants (20 ins or E20I) is the fact these alterations are heterogeneous, encompassing a diversity of amino acid insertions/deletions. In addition to E20I mutations, a number of other genetic alterations of the receptor, specifically point mutations leading to single amino acid substitutions, have been associated with the development of a variety of cancers, including lung cancer. Although the resistance mechanisms associated with each of these mutations are not fully understood, it is believed that the mutations may share a commonality in promoting ligand-independent activation of the kinases. Further investigation of the underlying mechanisms and development of TKIs tailored to these mutants are needed.

Other aggressive, refractory cancers exhibiting ErbB2 overexpression have been observed to harbor NRG1 gene re-arrangements resulting in novel fusion proteins. NRG1 gene fusions may induce overproduction of neuregulin-1, the cognate ligand for ErbB3. The simultaneous overexpression of ErbB2 and overproduction of neuregulin-1 may lead to excess activation of ErbB2-ErbB3 heterodimers and resultant hyperplasia.

Accordingly, there remains a need for new therapeutics for the treatment of cancers driven by dysregulated ErbB2 receptor kinase activity, not only with improved safety and selectivity for ErbB2 over EGFR, but also for addressing mutation-associated sub-variants of ErbB2 (e.g., E20I mutations and NRG1 gene fusions) with enhanced potency.

### SUMMARY OF THE INVENTION

In one aspect, provided is a compound of formula (I)

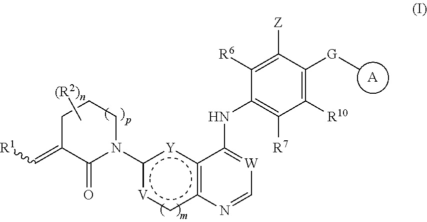

or a pharmaceutically acceptable salt, solvate, hydrate, or co-crystal thereof, or a mixture of any of the foregoing, wherein:
Ring A is

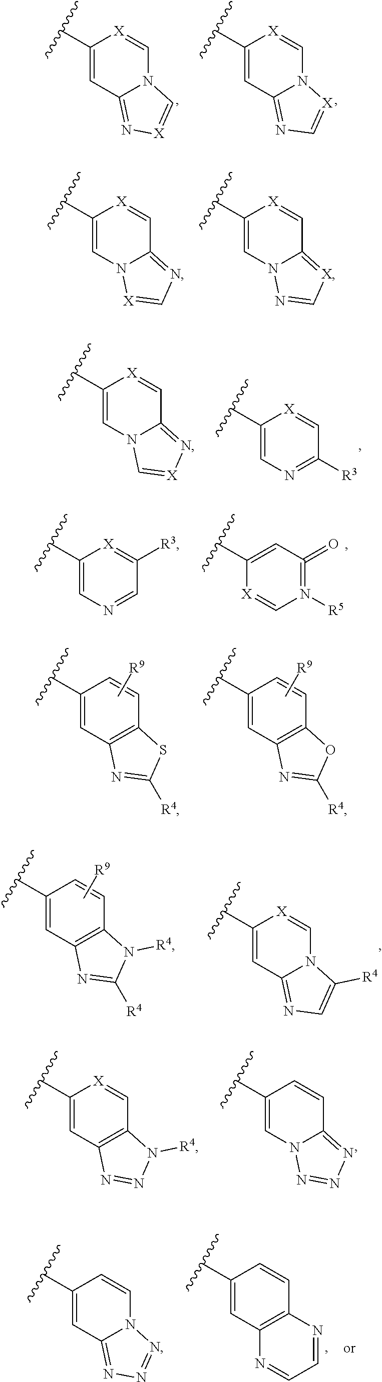

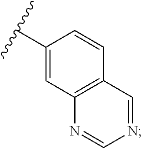 [SMILES: *c1ccc2cncnc2c1]

V is N, S, or C—R⁸;
W is N or C—CN;
each X is independently N or CH;
G is —CR^{g1}R^{g2}—, —O—, or —S—, wherein R^{g1} and R^{g2} are independently —H or —F;
Y is N or C—R^{y}, wherein R^{y} is —H or —F;
Z is —H, halogen, —OCH₃, —C≡CH, or C₁-C₂ alkyl;
R¹ is —H, C₁-C₃ alkyl, or 3- to 7-membered heterocycloalkyl,
wherein the C₁-C₃ alkyl is optionally substituted by 1-4 substituents selected from the group consisting of —F, —OH, a 3- to 7-membered carbon-linked N-heterocycloalkyl, and —NR^{1a}R^{1b} wherein each R^{1a} and R^{1b} is independently hydrogen, —CD₃, or C₁-C₃ alkyl, or wherein each pair of geminal R^{1a} and R^{1b} may be taken together with the nitrogen atom to which they are attached to form an N-heterocycloalkyl,
wherein the 3- to 7-membered heterocycloalkyl of R¹ is optionally substituted by —F, and wherein each heterocyclic nitrogen atom, if present, is independently optionally substituted with C₁-C₃ alkyl, and wherein R¹ may be cis or trans when R¹ is not —H;
m is an integer 0 or 1, wherein if m=0, then V is S, and if m=1, then V is N or CR⁸;
n is an integer 0, 1, or 2;
p is an integer 0 or 1;
each R² is independently —H or C₁-C₃ alkyl, wherein the C₁-C₃ alkyl is optionally substituted by 1-4 substituents selected from the group consisting of —F, —OH, —O(C₁-C₃ alkyl), a 3- to 7-membered carbon-linked N-heterocycloalkyl, and NR^{1a}R^{1b} wherein the C₁-C₃ alkyl of the —O(C₁-C₃ alkyl) is optionally substituted by —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl; or
two R² groups on the same carbon atom may be taken together with the carbon atom to which they are attached to form a C₃-C₆ spirocycloalkyl or 4- to 6-membered spiroheterocycloalkyl containing 1-2 ring heteroatoms selected from the group consisting of N, O, and S; or
two R² groups on vicinal carbon atoms may be taken together with the two carbon atoms to form a fused C₃-C₆ cycloalkyl;
R³ is —H, —F, —CD₃, C₁-C₃ alkyl, —CF₂H, —CN, —OR⁴, —SR⁴, —S(O)(C₁-C₃ alkyl), or —S(O)₂(C₁-C₃ alkyl);
each R⁴ is independently —H, —CD₃, C₁-C₃ alkyl, —CF₂H, —CF₃, or cyclopropyl;
R⁵ is C₁-C₃ alkyl, —CD₃, —CF₂H, allyl, —CH₂-cyclopropyl, cyclopropyl, or —OR⁴;
R⁶ is —H, -halogen, or C₁-C₃ alkyl;
R⁷ is —H, -halogen, or C₁-C₃ alkyl;
R⁸ is —H, —F, C₁-C₃ alkyl, or —O(C₁-C₃ alkyl),
wherein the C₁-C₃ alkyl and the the C₁-C₃ alkyl of the —O(C₁-C₃ alkyl) are each optionally substituted by 1-4 substituents selected from the group consisting of —F, —OH, —OR^{8a}, and —NR^{8a}R^{8b}, wherein each R^{8a} and R^{8b} are independently H or C₁-C₃ alkyl, or wherein each pair of geminal R^{8a} and R^{8b} may be taken together with the nitrogen atom to which they are attached to form an N-heterocycloalkyl wherein the N-heterocycloalkyl is optionally substituted by C₁-C₃ alkyl;
R⁹ is —H, halogen, —CN, C₁-C₃ alkyl, —CF₂H, —CF₃, cyclopropyl, —O(C₁-C₃ alkyl), or —O-cyclopropyl; and
R¹⁰ is —H or halogen.

In some embodiments, p is 0. In other embodiments, p is 1. In some embodiments, m is 0. In other embodiments, m is 1. In some embodiments, the compound of formula (I) is a compound of formula (II-a):

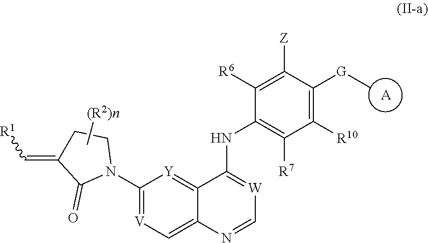

or a pharmaceutically acceptable salt, solvate, hydrate, or co-crystal thereof, or a mixture of any of the foregoing. In other embodiments, the compound of formula (I) is a compound of formula (II-b):

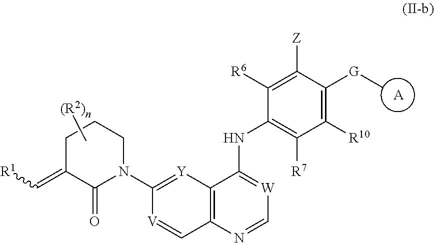

or a pharmaceutically acceptable salt, solvate, hydrate, or co-crystal thereof, or a mixture of any of the foregoing. In other embodiments, the compound of formula (I) is a compound of formula (II-c):

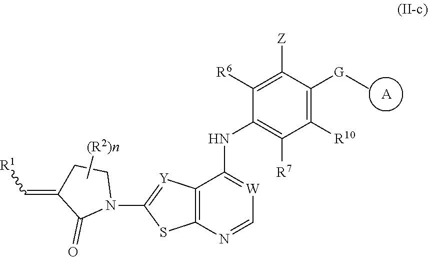

or a pharmaceutically acceptable salt, solvate, hydrate, or co-crystal thereof, or a mixture of any of the foregoing. In other embodiments, the compound of formula (I) is a compound of formula (II-d):

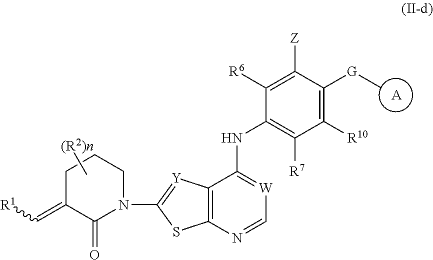

or a pharmaceutically acceptable salt, solvate, hydrate, or co-crystal thereof, or a mixture of any of the foregoing. In other embodiments, which may be combined with any of the preceding embodiments, Ring A is

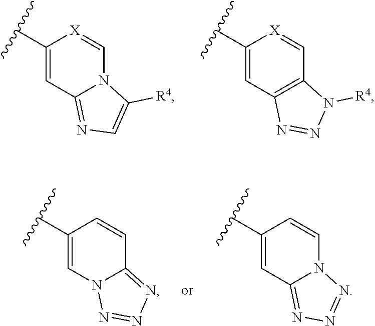

In one aspect, provided is a compound of formula (III)

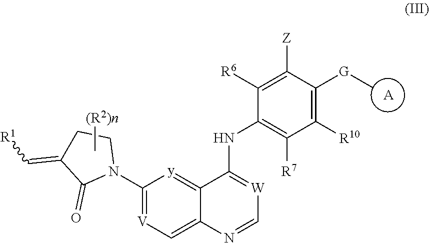

or a pharmaceutically acceptable salt, solvate, hydrate, or co-crystal thereof, or a mixture of any of the foregoing, wherein:
ring A is

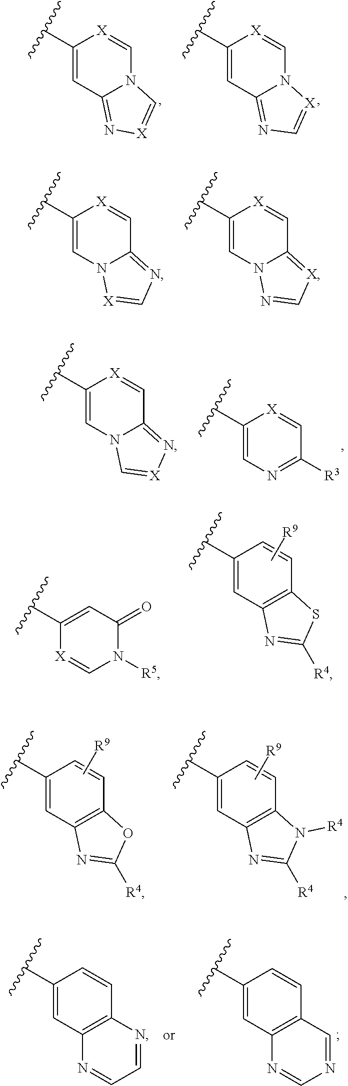

### V is N or C—R⁸;

W is N or C—CN;
each X is independently N or CH;
G is —CR^{g1}R^{g2}—, —O—, or —S—, wherein R^{g1} and R^{g2} are independently —H or —F;
Y is N or C—R^{y}, wherein R^{y} is —H or —F;
Z is —H, —F, —Cl, or C₁-C₂ alkyl;
R¹ is —H or C₁-C₃ alkyl,
wherein the C₁-C₃ alkyl is optionally substituted by 1-4 substituents selected from the group consisting of —F, —OH, a 3- to 7-membered carbon-linked N-heterocycloalkyl, and —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl, and wherein R¹ may be cis or trans when R¹ is not —H;
n is an integer 0, 1, or 2;
each R² is independently —H or C₁-C₃ alkyl, wherein the C₁-C₃ alkyl is optionally substituted by 1-4 substituents selected from the group consisting of —F, —OH, —O(C₁-C₃ alkyl), a 3- to 7-membered carbon-linked N-heterocycloalkyl, and NR^{1a}R^{1b} wherein the C₁-C₃ alkyl of the —O(C₁-C₃ alkyl) is optionally substituted by —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl; or
two R² groups on the same carbon atom may be taken together with the carbon atom to which they are attached to form a C₃-C₆ spirocycloalkyl or 4- to 6-membered spiroheterocycloalkyl containing 1-2 ring heteroatoms selected from the group consisting of N, O, and S; or
two R² groups on vicinal carbon atoms may be taken together with the two carbon atoms to form a fused C₃-C₆ cycloalkyl;
R³ is —H, —CD₃, C₁-C₃ alkyl, —CF₂H, —CN, —OR⁴, —SR⁴, —S(O)(C₁-C₃ alkyl), or —S(O)₂(C₁-C₃ alkyl);
each R⁴ is independently —H, C₁-C₃ alkyl, —CF₂H, —CF₃, or cyclopropyl;
R⁵ is C₁-C₃ alkyl, —CD₃, —CF₂H, allyl, —CH₂-cyclopropyl, cyclopropyl, or —OR⁴;
R⁶ is —H or —F;
R⁷ is —H or —F;
R⁸ is —H, —F, or —O(C₁-C₃ alkyl), wherein the C₁-C₃ alkyl is optionally substituted by 1-4 substituents selected from the group consisting of —F, —OH, —OR^{8a}, and —NR^{8a}R^{8b}, wherein each R^{8a} and R^{8b} are independently H or C₁-C₃ alkyl, or wherein each pair of geminal R^{8a} and R^{8b} may be taken together with the nitrogen atom to which they are attached to form an N-heterocycloalkyl wherein the N-heterocycloalkyl is optionally substituted by C₁-C₃ alkyl; and
R⁹ is —H, halogen, —CN, C₁-C₃ alkyl, —CF₂H, —CF₃, cyclopropyl, —O(C₁-C₃ alkyl), or —O-cyclopropyl.

In some embodiments of the present aspect, Ring A is

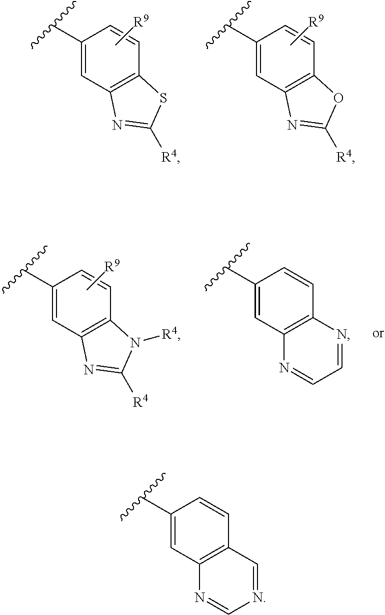

In some embodiments, which may be combined with the preceding embodiment, Ring A is

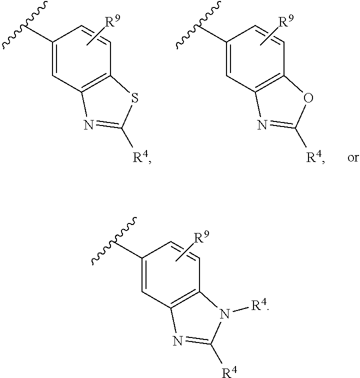

In other embodiments, which may be combined with any of the preceding embodiments, Ring A is

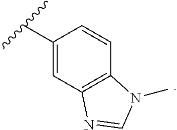 [SMILES: *c1ccc2c(c1)ncn2C]

In still other embodiments, which may be combined with any of the preceding embodiments, R⁸ is O(C₁-C₃ alkyl), wherein the C₁-C₃ alkyl is substituted with —NR^{1a}R^{1b}.

In some embodiments, the compound of formula (I), the compound of formula (II-a), the compound of formula (III) is a compound of formula (IV), or a pharmaceutically acceptable salt, solvate, hydrate, or co-crystal thereof, or a mixture of any of the foregoing.

In another aspect, provided is a compound of formula (IV)

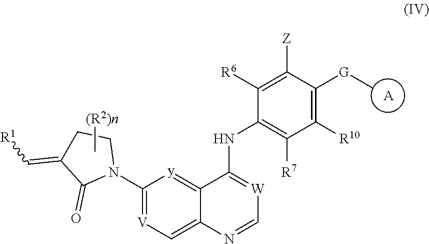

or a pharmaceutically acceptable salt, solvate, hydrate, or co-crystal thereof, or a mixture of any of the foregoing, wherein:
ring A is

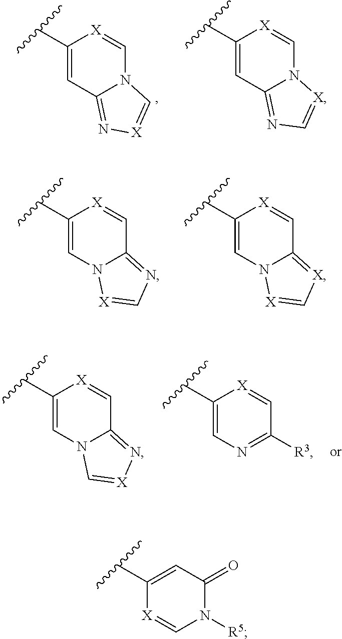

V is N or C—R⁸;
W is N or C—CN;
each X is independently N or CH;
G is —CR^{g1}R^{g2}—, —O—, or —S—, wherein R^{g1} and R^{g2} are independently —H or —F;
Y is N or C—R^{y}, wherein R^{y} is —H or —F;
Z is —H, —F, —Cl, or C₁-C₂ alkyl;
R¹ is —H or C₁-C₃ alkyl,
wherein the C₁-C₃ alkyl is optionally substituted by 1-4 substituents selected from the group consisting of —F, —OH, a 3- to 7-membered carbon-linked N-heterocycloalkyl, and —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl, and wherein R¹ may be cis or trans when R¹ is not —H;
n is an integer 0, 1, or 2;
each R² is independently —H or C₁-C₃ alkyl, wherein the C₁-C₃ alkyl is optionally substituted by 1-4 substituents selected from the group consisting of —F, —OH, a 3- to 7-membered carbon-linked N-heterocycloalkyl, and —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl; or
two R² groups on the same carbon atom may be taken together with the carbon atom to which they are attached to form a C₃-C₆ spirocycloalkyl or 4- to 6-membered spiroheterocycloalkyl containing 1-2 ring heteroatoms selected from the group consisting of N, O, and S; or
two R² groups on vicinal carbon atoms may be taken together with the two carbon atoms to form a fused C₃-C₆ cycloalkyl;
R³ is —H, —CD₃, C₁-C₃ alkyl, —CF₂H, —CN, —OR⁴, —SR⁴, —S(O)(C₁-C₃ alkyl), or —S(O)₂(C₁-C₃ alkyl);
R⁴ is —H, C₁-C₃ alkyl, —CF₂H, —CF₃, or cyclopropyl;
R⁵ is C₁-C₃ alkyl, —CD₃, —CF₂H, allyl, —CH₂-cyclopropyl, cyclopropyl, or —OR⁴;
R⁶ is —H or —F;
R⁷ is —H or —F, and
R⁸ is —H or —F.

In some embodiments of the present aspect, Ring A is

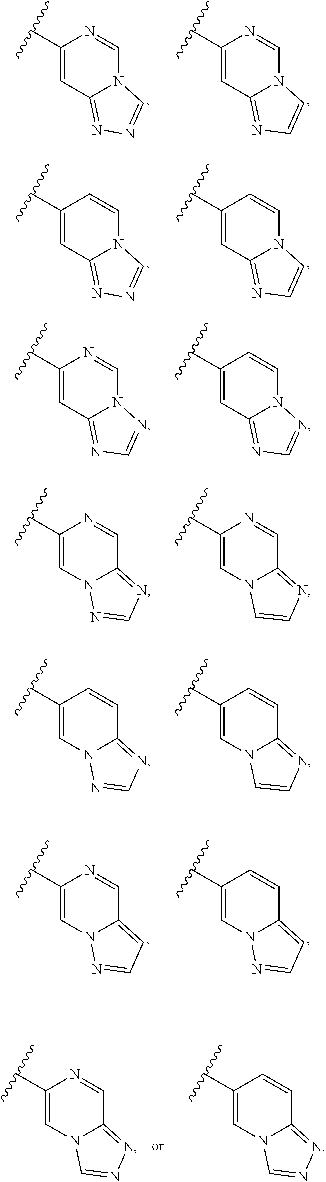

In some embodiments of the present aspect, ring A is

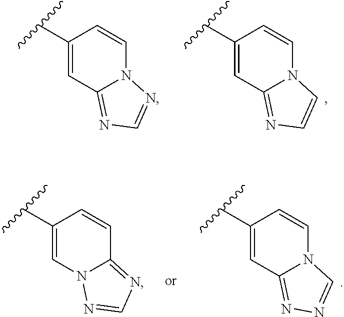 [SMILES: *c1ccn2ncnc2c1 ; *c1ccn2ccnc2c1 ; *c1ccc2ncnn2c1 ; *c1ccn2cnnc2c1]

In certain embodiments of the present aspect, ring A

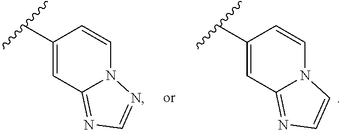 [SMILES: *c1ccn2ncnc2c1 ; "or" ; *c1ccn2ccnc2c1 ; "."]

In some embodiments of the present aspect, ring A is

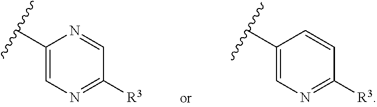

In some embodiments, R³ is —H, —CD₃, C₁-C₂ alkyl, or —CF₂H. In certain embodiments, R³ is —CH₃ or —CH₂CH₃.

In some embodiments of the present aspect, ring A is

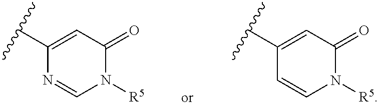

In certain embodiments, R₅ is C₁-C₂ alkyl, —CD₃, or —CF₂H. In certain embodiments, R₅ is —CH₃ or CF₂H.

In some embodiments of the present aspect, ring A is

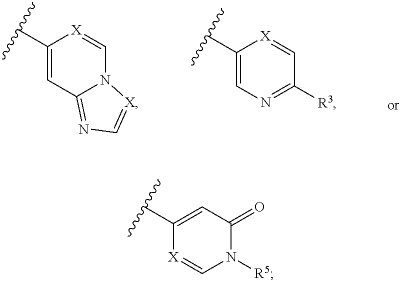

R³ is H, —CD₃, C₁-C₂ alkyl, or —CF₂H, and R⁵ is C₁-C₂ alkyl, —CD₃, or —CF₂H. In certain embodiments, R³ is —CH₃ or —CF₂H, and R⁵ is —CH₃ or —CF₂H.

In some embodiments of the present aspect, which may be combined with any of the preceding embodiments, Z is —H, —F, —Cl, or —CH₃. In certain embodiments, Z is —CH₃.

In some embodiments, R¹ is —H. In some embodiments, R¹ is C₁-C₃ alkyl optionally substituted by —OH or —N(C₁-C₃ alkyl)(C₁-C₃ alkyl). In certain embodiments, R¹ is —CH₃, —CH₂OH, —(CH₂)₂OH or —CH₂N(CH₃)₂. In still other embodiments, R¹ is —CH₃.

In yet further embodiments, which may be combined with any of the preceding embodiments, R^{y} is —H or —F. In certain embodiments, R^{y} is —H. In certain embodiments, R^{y} is —F. In some embodiments, which may be combined with any of the preceding embodiments, W is N. In other embodiments, W is C—CN. In some embodiments, which may be combined with any of the preceding embodiments, V is N. In other embodiments, V is C—R⁸. In some embodiments, n is an integer 1 or 2. In still additional embodiments, which may be combined with any of the preceding embodiments, n is 2, and the two R² groups are present on the same carbon atom and are taken together with the carbon atom to which they are attached to form a C₃-C₆ spirocycloalkyl or 4- to 6-membered spiroheterocycloalkyl containing 1-2 ring heteroatoms selected from the group consisting of N, O, and S, or the two R² groups are on vicinal carbon atoms and are taken together with the two carbon atoms to form a fused C₃-C₆ cycloalkyl. In some embodiments, n is 2, and the two R² groups are present on the same carbon atom and are taken together with the carbon atom to which they are attached to form a C₃-C₆ spirocycloalkyl. In some embodiments, n is 2, and the two R² groups are on vicinal carbon atoms and are taken together with the two carbon atoms to form a fused C₃-C₆ cycloalkyl. In certain embodiments, each R² is independently —H or C₁-C₃ alkyl. In some embodiments, which may be combined with any of the preceding embodiments, G is —O—. In other embodiments, G is —CH₂—. In yet other embodiments, G is S. In other embodiments, R⁶ is —H. In some embodiments, R⁶ is —F. In still further embodiments, R⁷ is —H. In yet other embodiments, R⁷ is —F.

In another aspect, provided herein are compounds as described herein in Table 1. In yet another aspect, provided herein are pharmaceutical compositions comprising a compound of formula (I), formula (II-a), formula (II-b), formula (II-c), formula (II-d), formula (III), formula (IV), or of Table 1 as described herein, or a pharmaceutically acceptable salt, solvate, hydrate, or co-crystal thereof, or a mixture of any of the foregoing, and at least one pharmaceutically acceptable excipient.

In yet another aspect, provided herein are synthetic methods and intermediates thereof for the compounds of formula (I), formula (II-a), formula (II-b), formula (II-c), formula (II-d), formula (III), formula (IV), or of Table 1, or a pharmaceutically acceptable salt, solvate, hydrate, or co-crystal thereof, or a mixture of any of the foregoing, as described herein.

In one aspect, provided herein is a method of inhibiting kinase activity of a human receptor tyrosine kinase ErbB2 or a mutant form of human ErbB2 comprising contacting the ErbB2 or the mutant form with a therapeutically effective amount of a compound of formula (I), formula (II-a), formula (II-b), formula (II-c), formula (II-d), formula (III), formula (IV), or of Table 1, or a pharmaceutically acceptable salt, solvate, hydrate, or co-crystal thereof, or a mixture of any of the foregoing, as described herein, or a therapeutically effective amount of the pharmaceutical composition as described herein. In some embodiments of the present aspect, the mutant form of human ErbB2 comprises a mutation in Exon 20. In further embodiments of the present aspect, the mutant form of human ErbB2 comprises one or more mutations that introduce amino acid deletions and/or insertions selected from the group consisting of: A775_A776insYVMA, G778_P780insGSP, G776delinsVC, P780_Y781insGSP, M774delinsWLV, A775_G776insSVMA, A775_G776insI, G776delinsLC, G778_S779InsCPG, and V777_G778insGSP. In other embodiments of the present aspect, the mutant form of human ErbB2 comprises a disease-associated point mutation in ErbB2. In still further embodiments of the present aspect, the mutant form of human ErbB2 comprises one or more point mutations in ErbB2 that introduce amino acid substitutions selected from the group consisting of P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, N1219S, and A1232fs. In other embodiments, the mutant form of human ErbB2 comprises one or more point mutations in ErbB2 that introduce (a) an amino acid substitution selected from the group consisting of are P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, and N1219S; or a frameshift at A1232.

In yet another aspect, provided herein is a method of treating a patient having a cancer, comprising administering to the patient a therapeutically effective amount of a compound of formula (I), formula (II-a), formula (II-b), formula (II-c), formula (II-d), formula (III), formula (IV), or of Table 1, or a pharmaceutically acceptable salt, solvate, hydrate, or co-crystal thereof, or a mixture of any of the foregoing, as described herein, or a therapeutically effective amount of the pharmaceutical composition as described herein. In some embodiments of the present aspect, the cancer comprises cells or cell tissue having increased ErbB2 kinase activity. In some embodiments of the present aspect, the cancer comprises cells or cell tissue having increased ErbB2 kinase activity as compared to a control. In certain embodiments, the cancer comprises cells or cell tissue having increased ErbB2 kinase activity as compared to ErbB2 kinase activity in control cell or in control cell tissue. In further embodiments of the present aspect, the cancer comprises cells or cell tissue having one or more mutations in Exon 20 of the ErbB2. In certain embodiments, the cancer comprises cells or cell tissue having one or more mutations in Exon 20 of the ErbB2 that introduce amino acid deletions and/or insertions selected from the group consisting of A775_A776insYVMA, G778_P780insGSP, G776delinsVC, P780_Y781insGSP, M774delinsWLV, A775_G776insSVMA, A775_G776insI, G776delinsLC, G778_S779InsCPG, and V777_G778insGSP. In other embodiments, the cancer comprises cells or cell tissue having one or more disease-associated point mutations in ErbB2. In certain other embodiments, the cancer comprises cells or cell tissue having one or more point mutations that introduce amino acid substitutions selected from the group consisting of are P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, N1219S, and A1232fs. In certain embodiments, the cancer comprises cells or cell tissue having one or more point mutations that introduce (a) an amino acid substitution selected from the group consisting of are P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, and N1219S; or a frameshift at A1232.

In some embodiments of the present aspect, which may be combined with any of the preceding embodiments, the cancer is lung, glioma, skin, head and neck, salivary gland, breast, esophageal, liver, stomach (gastric), uterine, cervical, biliary tract, pancreatic, colorectal, renal, bladder or prostate cancer. In certain embodiments, the cancer is non-small cell lung cancer. In still other embodiments, which may be combined with any of the preceding embodiments, the patient has received at least one, at least two, or at least three prior therapies for the cancer. In certain embodiments, one or more of the prior therapies selected from the group consisting of lapatinib, neratinib, afatinib, pyrotinib, poziotinib, TAK-788, and tucatinib. In some embodiments, the method further comprises administering one or more additional anti-cancer agents.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

### I. Definitions

As used herein, the following definitions shall apply unless otherwise indicated.

Further, if any term or symbol used herein is not defined as set forth below, it shall have its ordinary meaning in the art.

The term “excipient” as used herein means an inert or inactive substance that may be used in the production of a drug or pharmaceutical, such as a tablet containing a compound of the present disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

The terms “individual”, “subject” and “patient” refer to mammals and includes humans and non-human mammals. Examples of patients include, but are not limited to, mice, rats, hamsters, guinea pigs, pigs, rabbits, cats, dogs, goats, sheep, cows, and humans. In some embodiments, patient refers to a human.

As used herein, the term “mammal” includes, but is not limited to, humans, mice, rats, guinea pigs, monkeys, dogs, cats, horses, cows, pigs, and sheep.

“Pharmaceutically acceptable” refers to safe and non-toxic, and suitable for in vivo or for human administration.

As used herein, the term “alkyl”, by itself or as part of another substituent, means, unless otherwise stated, a straight or branched chain hydrocarbon radical, having the number of carbon atoms designated (i.e., C₁-C₆ means one to six carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. In some embodiments, the term “alkyl” may encompass C₁-C₆ alkyl, C₂-C₆ alkyl, C₃-C₆ alkyl, C₄-C₆ alkyl, C₅-C₆ alkyl, C₁-C₅ alkyl, C₂-C₅ alkyl, C₃-C₅ alkyl, C₄-C₅ alkyl, C₁-C₄ alkyl, C₂-C₄ alkyl, C₃-C₄ alkyl, C₁-C₃ alkyl, C₂-C₃ alkyl, or C₁-C₂ alkyl.

The term “cycloalkyl” refers to hydrocarbon rings having the indicated number of ring atoms (e.g., C₃-C₆ cycloalkyl means 3-6 carbons) and being fully saturated or having no more than one double bond between ring vertices. In some embodiments, “cycloalkyl” encompasses C₃-C₇ cycloalkyl, C₄-C₇ cycloalkyl, C₅-C₇ cycloalkyl, C₅-C₇ cycloalkyl, C₃-C₆ cycloalkyl, C₄-C₆ cycloalkyl, C₅-C₆ cycloalkyl, C₃-C₅ cycloalkyl, C₄-C₅ cycloalkyl, or C₃-C₄ cycloalkyl. In some embodiments, the term “cycloalkyl” may be further described as a “spirocycloalkyl” or a “fused cycloalkyl”. The term “spirocycloalkyl” refers to hydrocarbon rings having the indicated number of ring atoms (e.g., C₃-C₆ cycloalkyl means 3-6 carbons) and being fully saturated or having no more than one double bond between ring vertices, wherein the hydrocarbon ring is attached to the rest of the molecule at a single ring vertex (e.g., ring carbon atom) by two covalent bonds. The term “fused “cycloalkyl” refers to hydrocarbon rings having the indicated number of ring atoms (e.g., C₃-C₆ cycloalkyl means 3-6 carbons) and being fully saturated or having no more than one double bond between ring vertices, wherein the hydrocarbon ring is attached to the rest of the molecule at two ring vertices (e.g. two carbon atoms) by two covalent bonds.

The term “heterocycloalkyl”, “heterocyclic”, “heterocyclyl”, or “heterocycle” refers to a cycloalkyl radical group having the indicated number of ring atoms (e.g., 5-6 membered heterocycloalkyl) that contain from one to five heteroatoms selected from the group consisting of N, O, and S, wherein the nitrogen and sulfur atoms are optionally oxidized, nitrogen atom(s) are optionally quaternized, as ring atoms. Unless otherwise stated, a “heterocycloalkyl,” “heterocyclic,” or “heterocycle” ring can be a monocyclic, a bicyclic, spirocyclic or a polycylic ring system. Non-limiting examples of “heterocycloalkyl,” “heterocyclic,” or “heterocycle” rings include pyrrolidine, piperidine, N-methylpiperidine, imidazolidine, pyrazolidine, butyrolactam, valerolactam, imidazolidinone, hydantoin, dioxolane, phthalimide, piperidine, pyrimidine-2,4(1H,3H)-dione, 1,4-dioxane, morpholine, thiomorpholine, thiomorpholine-5-oxide, thiomorpholine-S,S-oxide, piperazine, pyran, pyridone, 3-pyrroline, thiopyran, pyrone, tetrahydrofuran, tetrhydrothiophene, quinuclidine, tropane and the like. A “heterocycloalkyl,” “heterocyclic,” or “heterocycle” group can be attached to the remainder of the molecule through one or more ring carbons or heteroatoms. In some embodiments, “heterocycloalkyl” encompasses 4- to 8-membered heterocycloalkyl, 5- to 8-membered heterocycloalkyl, 6- to 8-membered heterocycloalkyl, 7- to 8-membered heterocycloalkyl, 4- to 7-membered heterocycloalkyl, 5- to 7-membered heterocycloalkyl, 6- to 7-membered heterocycloalkyl, 4- to 6-membered heterocycloalkyl, 5- to 6-membered heterocycloalkyl, or 4- to 5-membered heterocycloalkyl. The term “alkylene” by itself or as part of another substituent means a divalent radical derived from an alkane, as exemplified by —CH₂CH₂CH₂CH₂—. Typically, an alkyl (or alkylene) group will have from 1 to 24 carbon atoms. In some embodiments, an alkyl (or alkylene) group will have 10 or fewer carbon atoms.

The term “heteroalkylene” by itself or as part of another substituent means a divalent radical, saturated or unsaturated or polyunsaturated, derived from heteroalkyl, as exemplified by —CH₂—CH₂—S—CH₂CH₂—, —CH₂—S—CH₂—CH₂—NH—CH₂—, —O—CH₂—CH═CH—, —CH₂—CH═C(H)CH₂—O—CH₂— and —S—CH₂—C≡C—. For heteroalkylene groups, heteroatoms can also occupy either or both of the chain termini (e.g., alkyleneoxy, alkylenedioxy, alkyleneamino, alkylenediamino, and the like).

The term “heterocycloalkylene” by itself or as part of another substituent means a divalent radical, saturated or unsaturated or polyunsaturated, derived from heterocycloalkyl. For heterocycloalkylene groups, heteroatoms can also occupy either or both of the chain termini.

The terms “alkoxy” and “alkylamino” are used in their conventional sense, and refer to those alkyl groups attached to the remainder of the molecule via an oxygen atom or an amino group, respectively.

The term “heterocycloalkoxy” refers to a heterocycloalkyl-O— group in which the heterocycloalkyl group is as previously described herein.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “C₁-C₄ haloalkyl” is mean to include trifluoromethyl, 2,2,2-trifluoroethyl, 4-chlorobutyl, 3-bromopropyl, difluoromethyl, and the like.

The term “haloalkyl-OH” refers to a haloalkyl group as described above which is also substituted by one or more hydroxyl groups. The term “haloalkyl-OH” is meant to include haloalkyl substituted by one hydroxyl group, as well as haloalkyl substituted by multiple hydroxyl groups. For example, the term “haloalkyl-OH” includes —CH(F)OH, —CH₂CFHCH₂OH, —CH(OH)CF₃, and the like.

The term “alkyl-OH” refers to an alkyl substituted by one or more hydroxyl groups. The term “alkyl-OH” is meant to include alkyl substituted by one hydroxyl group, as well as alkyl substituted by multiple hydroxyl groups. For example, the term “alkyl-OH” includes —CH₂OH, —CH(OH)CH₃, —CH₂CH₂OH, and the like.

The term “aryl” means, unless otherwise stated, a polyunsaturated, typically aromatic, hydrocarbon group, which can be a single ring or multiple rings (up to three rings) which are fused together. In some embodiments, “aryl” encompasses C₆-C₁₄ aryl, C₈-C₁₄ aryl, C₁₀-C₁₄ aryl, C₁₂-C₁₄ aryl, C₆-C₁₂ aryl, C₈-C₁₂ aryl, C₁₀-C₁₂ aryl, C₆-C₁₀ aryl, C₅-C₁₀ aryl, or C₆-C₈ aryl. The term “heteroaryl” refers to aryl groups (or rings) that contain from one to five heteroatoms selected from the group consisting of N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl groups include phenyl, naphthyl and biphenyl, while non-limiting examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. In some embodiments, the term “heteroaryl” encompasses 5- to 10-membered heteroaryl, 6- to 10-membered heteroaryl, 7- to 10-membered heteroaryl, 8- to 10-membered heteroaryl, 9- to 10-membered heteroaryl, 5- to 9-membered heteroaryl, 6- to 9-membered heteroaryl, 7- to 9-membered heteroaryl, 8- to 9-membered heteroaryl, 5- to 8-membered heteroaryl, 6- to 8-membered heteroaryl, 7- to 8-membered heteroaryl, 5- to 7-membered heteroaryl, 6- to 7-membered heteroaryl, or 5- to 6-membered heteroaryl.

The above terms (e.g., “alkyl,” “aryl” and “heteroaryl”), in some embodiments, will include both substituted and unsubstituted forms of the indicated radical. The term “substituted” means that the specified group or moiety bears one or more substituents including, but not limited to, substituents such as alkoxy, acyl, acyloxy, alkoxycarbonyl, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, alkyl, alkenyl, alkynyl, heterocycloalkyl, heterocycloalkenyl, aralkyl, aminosulfonyl, sulfonylamino, sulfonyl, oxo and the like. The term “unsubstituted” means that the specified group bears no substituents. Where the term “substituted” is used to describe a structural system, the substitution is meant to occur at any valency-allowed position on the system. When a group or moiety bears more than one substituent, it is understood that the substituents may be the same or different from one another. In some embodiments, a substituted group or moiety bears from one to five substituents. In some embodiments, a substituted group or moiety bears one substituent. In some embodiments, a substituted group or moiety bears two substituents. In some embodiments, a substituted group or moiety bears three substituents. In some embodiments, a substituted group or moiety bears four substituents. In some embodiments, a substituted group or moiety bears five substituents.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible, and/or inherently unstable. It will also be understood that where a group or moiety is optionally substituted, the disclosure includes both embodiments in which the group or moiety is substituted and embodiments in which the group or moiety is unsubstituted.

As used herein, the term “heteroatom” is meant to include oxygen (O), nitrogen (N), sulfur (S), boron (B), and silicon (Si).

As used herein, the term “chiral” refers to molecules which have the property of non-superimposability of the mirror image partner, while the term “achiral” refers to molecules which are superimposable on their mirror image partner.

As used herein, the term “stereoisomers” refers to compounds which have identical chemical constitution, but differ with regard to the arrangement of the atoms or groups in space.

As used herein, a wavy line “ [symbol] ” that intersects a bond in a chemical structure indicates the point of attachment of the atom to which the wavy bond is connected in the chemical structure to the remainder of a molecule, or to the remainder of a fragment of a molecule.

As used herein, the representation of a group (e.g., X^{a}) in parenthesis followed by a subscript integer range (e.g., (X^{a})₀₋₁) means that the group can have the number of occurrences as designated by the integer range. For example, (X^{a})₀₋₁ means the group X^{a} can be absent or can occur one time.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers can separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the present disclosure can contain asymmetric or chiral centers, and therefore exist in different stereoisomeric forms. It is intended that all stereoisomeric forms of the compounds of the present disclosure, including but not limited to, diastereomers, enantiomers and atropisomers, as well as mixtures thereof such as racemic mixtures, form part of the present disclosure. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

As used herein, the term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

As used herein, the term “solvate” refers to an association or complex of one or more solvent molecules and a compound of the present disclosure. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water. Certain compounds of the present disclosure can exist in unsolvated forms as well as solvated forms, including hydrated forms. In general, the solvated forms are equivalent to unsolvated forms and are intended to be encompassed within the scope of the present disclosure.

The term “co-crystal” as used herein refers to a solid that is a crystalline single phase material composed of two or more different molecular or ionic compounds generally in a stoichiometric ratio which are neither solvates nor simple salts. A co-crystal consists of two or more components that form a unique crystalline structure having unique properties. Co-crystals are typically characterized by a crystalline structure, which is generally held together by freely reversible, non-covalent interactions. As used herein, a co-crystal refers to a compound of the present disclosure and at least one other component in a defined stoichiometric ratio that form a crystalline structure.

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethyl silyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein, the term “pharmaceutically acceptable salts” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

Certain compounds of the present disclosure possess asymmetric carbon atoms (optical centers) or double bonds; the racemates, diastereomers, geometric isomers, regioisomers and individual isomers (e.g., separate enantiomers) are all intended to be encompassed within the scope of the present disclosure.

The compounds of the present disclosure can also contain unnatural proportions of atomic isotopes at one or more of the atoms that constitute such compounds. For example, the present disclosure also embraces isotopically-labeled variants of the present disclosure which are identical to those recited herein, but for the fact that one or more atoms are replaced by an atom having the atomic mass or mass number different from the predominant atomic mass or mass number usually found in nature for the atom. All isotopes of any particular atom or element as specified are contemplated within the scope of the compounds of the present disclosure and include isotopes of hydrogen, carbon, nitrogen, oxygen, phosphorous, sulfur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Certain isotopically labeled compounds of the present disclosure (e.g., those labeled with ³H or ¹⁴C) are useful in compound and/or substrate tissue distribution assays. Tritiated (³H) and carbon-14 (¹⁴C) isotopes are useful for their ease of preparation and detectability. Further substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵I, ¹³N, ¹¹C, and ¹⁸F are useful for positron emission tomography (PET) studies to examine substrate receptor occupancy. Isotopically labeled compounds of the present disclosure can generally be prepared by following procedures analogous to those disclosed in the Schemes and/or in the Examples herein below, by substituting an isotopically labeled reagent for a non-isotopically labeled reagent.

“Treating” or “treatment” of a disease in a patient refers to inhibiting the disease or arresting its development; or ameliorating or causing regression of the disease. As used herein, “treatment” or “treating” is an approach for obtaining beneficial or desired results including clinical results. For purposes of this disclosure, beneficial or desired results include, but are not limited to, one or more of the following: decreasing one more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delay or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of the present disclosure contemplate any one or more of these aspects of treatment.

“Preventing”, “prevention”, or “prophylaxis” of a disease in a patient refers to preventing the disease from occurring in a patient that is predisposed or does not yet display symptoms of the disease.

The phrase “therapeutically effective amount” means an amount of a compound of the present disclosure that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The terms “cancer” and “cancerous” refer to or describe the physiological condition in mammals that is typically characterized by unregulated cell growth.

It is appreciated that certain features of the present disclosure, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present invention and are disclosed herein just as if each and every such sub-combination of chemical groups was individually and explicitly disclosed herein.

### II. Compounds

In one aspect, provided is a compound of formula (I)

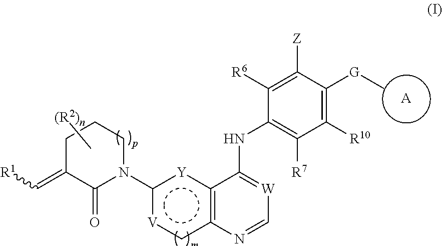

or a pharmaceutically acceptable salt, solvate, hydrate, or co-crystal thereof, or a mixture of any of the foregoing, wherein:
ring A is

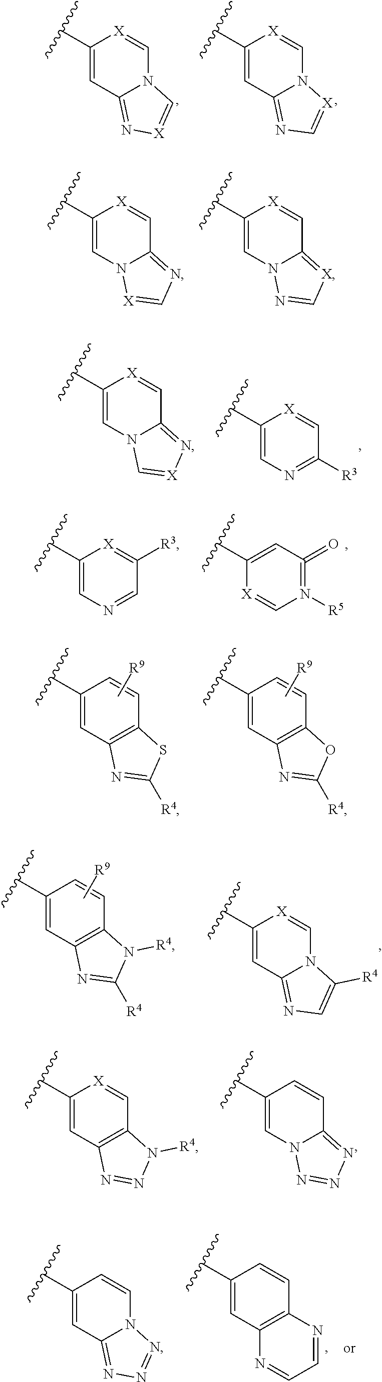

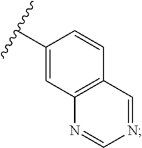 [SMILES: *c1ccc2cncnc2c1]

V is N, S, or C—R⁸;
W is N or C—CN;
each X is independently N or CH;
G is —CR^{g1}R^{g2}—, —O—, or —S—, wherein R^{g1} and R^{g2} are independently —H or —F;
Y is N or C—R^{y}, wherein R^{y} is —H or —F;
Z is —H, halogen, —OCH₃, —C≡CH, or C₁-C₂ alkyl;
R¹ is —H, C₁-C₃ alkyl, or 3- to 7-membered heterocycloalkyl,
wherein the C₁-C₃ alkyl is optionally substituted by 1-4 substituents selected from the group consisting of —F, —OH, a 3- to 7-membered carbon-linked N-heterocycloalkyl, and —NR^{1a}R^{1b} wherein each R^{1a} and R^{1b} is independently hydrogen, —CD₃, or C₁-C₃ alkyl, or wherein each pair of geminal R^{1a} and R^{1b} may be taken together with the nitrogen atom to which they are attached to form an N-heterocycloalkyl,
wherein the 3- to 7-membered heterocycloalkyl of R¹ is optionally substituted by —F, and wherein each heterocyclic nitrogen atom, if present, is independently optionally substituted with C₁-C₃ alkyl, and wherein R¹ may be cis or trans when R¹ is not —H;
m is an integer 0 or 1, wherein if m=0, then V is S, and if m=1, then V is N or CR⁸;
n is an integer 0, 1, or 2;
p is an integer 0 or 1;
each R² is independently —H or C₁-C₃ alkyl, wherein the C₁-C₃ alkyl is optionally substituted by 1-4 substituents selected from the group consisting of —F, —OH, —O(C₁-C₃ alkyl), a 3- to 7-membered carbon-linked N-heterocycloalkyl, and NR^{1a}R^{1b} wherein the C₁-C₃ alkyl of the —O(C₁-C₃ alkyl) is optionally substituted by —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl; or
two R² groups on the same carbon atom may be taken together with the carbon atom to which they are attached to form a C₃-C₆ spirocycloalkyl or 4- to 6-membered spiroheterocycloalkyl containing 1-2 ring heteroatoms selected from the group consisting of N, O, and S; or
two R² groups on vicinal carbon atoms may be taken together with the two carbon atoms to form a fused C₃-C₆ cycloalkyl;
R³ is —H, —F, —CD₃, C₁-C₃ alkyl, —CF₂H, —CN, —OR⁴, —SR⁴, —S(O)(C₁-C₃ alkyl), or —S(O)₂(C₁-C₃ alkyl);
each R⁴ is independently —H, —CD₃, C₁-C₃ alkyl, —CF₂H, —CF₃, or cyclopropyl;
R⁵ is C₁-C₃ alkyl, —CD₃, —CF₂H, allyl, —CH₂-cyclopropyl, cyclopropyl, or —OR⁴;
R⁶ is —H, -halogen, or C₁-C₃ alkyl;
R⁷ is —H, -halogen, or C₁-C₃ alkyl;
R⁸ is —H, —F, C₁-C₃ alkyl, or —O(C₁-C₃ alkyl),
wherein the C₁-C₃ alkyl and the C₁-C₃ alkyl of the —O(C₁-C₃ alkyl) are each optionally substituted by 1-4 substituents selected from the group consisting of —F, —OH, —OR^{8a}, and —NR^{8a}R^{8b}, wherein each R^{8a} and R^{8b} are independently H or C₁-C₃ alkyl, or wherein each pair of geminal R^{8a} and R^{8b} may be taken together with the nitrogen atom to which they are attached to form an N-heterocycloalkyl wherein the N-heterocycloalkyl is optionally substituted by C₁-C₃ alkyl;
R⁹ is —H, halogen, —CN, C₁-C₃ alkyl, —CF₂H, —CF₃, cyclopropyl, —O(C₁-C₃ alkyl), or —O-cyclopropyl; and
R¹⁰ is —H or halogen.

In some embodiments, m is 0. In other embodiments, m is 1. In some embodiments, p is 0. In other embodiments, p is 1. In some embodiments, m is 1 and the compound of formula (I) is a compound of formula (II-a) or a compound of formula (II-b). In other embodiments, m is 0 and the compound of formula (I) is a compound of formula (II-c) or a compound of formula (II-d). In some embodiments, p is 0 and the compound of formula (I) is a compound of formula (II-a) or a compound of formula (II-c). In other embodiments, p is 1 and the compound of formula (I) is a compound of formula (II-b) or a compound of formula (II-d).

In some embodiments, m is 1, p is 0, and the compound of formula (I) is a compound of formula (II-a):

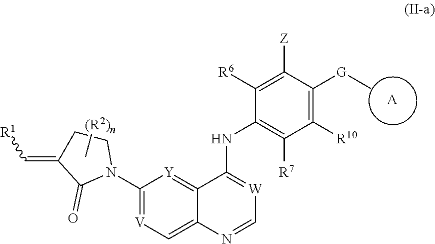

or pharmaceutically acceptable salt, solvate, hydrate, or co-crystal thereof, or a mixture of any of the foregoing. In other embodiments, m is 1, p is 1 and the compound of formula (I) is a compound of formula (II-b):

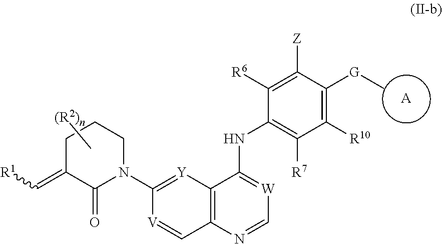

or pharmaceutically acceptable salt, solvate, hydrate, or co-crystal thereof, or a mixture of any of the foregoing. In yet other embodiments, m is 0, p is 0 and the compound of formula (I) is a compound of formula (II-c):

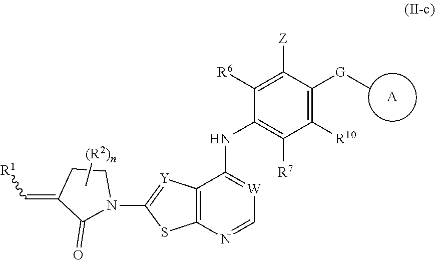

or pharmaceutically acceptable salt, solvate, hydrate, or co-crystal thereof, or a mixture of any of the foregoing. In still yet other embodiments, m is 0, p is 1 and the compound of formula (I) is a compound of formula (II-d):

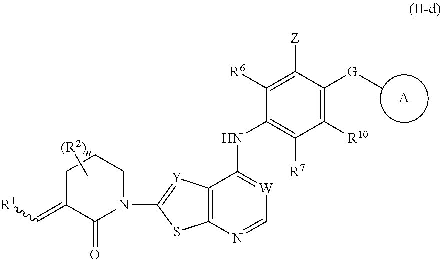

or pharmaceutically acceptable salt, solvate, hydrate, or co-crystal thereof, or a mixture of any of the foregoing.

In some embodiments, the compound of formula (I) or formula (II-a) is a compound of formula (III):

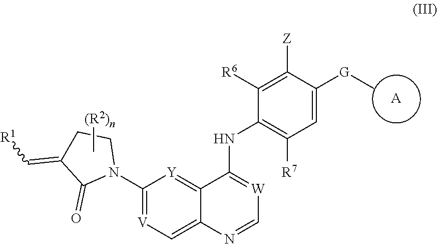

or pharmaceutically acceptable salt, solvate, hydrate, or co-crystal thereof, or a mixture of any of the foregoing.

In one aspect, provided herein is a compound of formula (III)

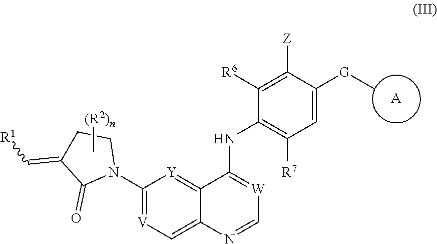

or pharmaceutically acceptable salt, solvate, hydrate, or co-crystal thereof, or a mixture of any of the foregoing, wherein:
ring A is

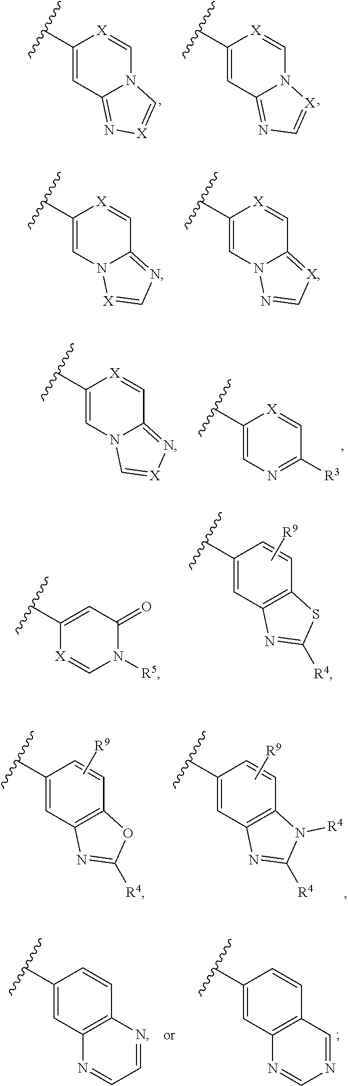

V is N or C—R⁸;
W is N or C—CN;
each X is independently N or CH;
G is —CR^{g1}R^{g2}—, —O—, or —S—, wherein R^{g1} and R^{g2} are independently —H or —F;
Y is N or C—R^{y}, wherein R^{y} is —H or —F;
Z is —H, —F, —Cl, or C₁-C₂ alkyl;
R¹ is —H or C₁-C₃ alkyl,
wherein the C₁-C₃ alkyl is optionally substituted by 1-4 substituents selected from the group consisting of —F, —OH, a 3- to 7-membered carbon-linked N-heterocycloalkyl, and —NR^{1a}R^{1b},
wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl, and wherein R¹ may be cis or trans when R¹ is not —H;
n is an integer 0, 1, or 2;
each R² is independently —H or C₁-C₃ alkyl, wherein the C₁-C₃ alkyl is optionally substituted by 1-4 substituents selected from the group consisting of —F, —OH, —O(C₁-C₃ alkyl), a 3- to 7-membered carbon-linked N-heterocycloalkyl, and NR^{1a}R^{1b} wherein the C₁-C₃ alkyl of the —O(C₁-C₃ alkyl) is optionally substituted by —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl; or
two R² groups on the same carbon atom may be taken together with the carbon atom to which they are attached to form a C₃-C₆ spirocycloalkyl or 4- to 6-membered spiroheterocycloalkyl containing 1-2 ring heteroatoms selected from the group consisting of N, O, and S; or
two R² groups on vicinal carbon atoms may be taken together with the two carbon atoms to form a fused C₃-C₆ cycloalkyl;
R³ is —H, —CD₃, C₁-C₃ alkyl, —CF₂H, —CN, —OR⁴, —SR⁴, —S(O)(C₁-C₃ alkyl), or —S(O)₂(C₁-C₃ alkyl);
each R⁴ is independently —H, C₁-C₃ alkyl, —CF₂H, —CF₃, or cyclopropyl;
R⁵ is C₁-C₃ alkyl, —CD₃, —CF₂H, allyl, —CH₂-cyclopropyl, cyclopropyl, or —OR⁴;
R⁶ is —H or —F;
R⁷ is —H or —F;
R⁸ is —H, —F, or —O(C₁-C₃ alkyl), wherein the C₁-C₃ alkyl is optionally substituted by 1-4 substituents selected from the group consisting of —F, —OH, —OR^{8a}, and —NR^{8a}R^{8b}, wherein each R^{8a} and R^{8b} are independently H or C₁-C₃ alkyl, or wherein each pair of geminal R^{8a} and R^{8b} may be taken together with the nitrogen atom to which they are attached to form an N-heterocycloalkyl wherein the N-heterocycloalkyl is optionally substituted by C₁-C₃ alkyl; and
R⁹ is —H, halogen, —CN, C₁-C₃ alkyl, —CF₂H, —CF₃, cyclopropyl, —O(C₁-C₃ alkyl), or —O-cyclopropyl.

In some embodiments, the compound of formula (I), the compound of formula (II-a), or the compound of formula (III) is a compound of formula (IV)

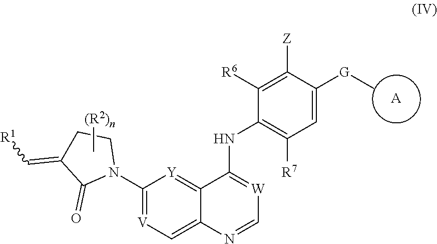

or pharmaceutically acceptable salt, solvate, hydrate, or co-crystal thereof, or a mixture of any of the foregoing, wherein:
ring A is

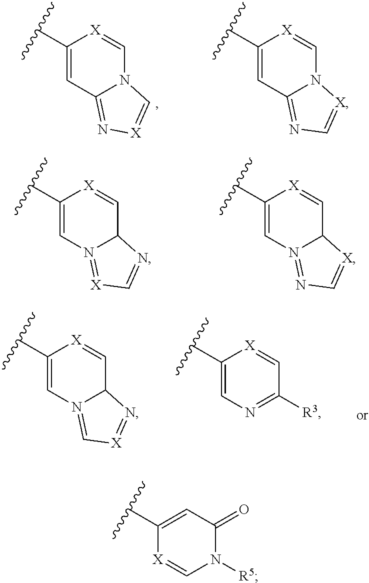

V is N or C—R⁸;
W is N or C—CN;
each X is independently N or CH;
G is —CR^{g1}R^{g2}—, —O—, or —S—, wherein R^{g1} and R^{g2} are independently —H or —F;
Y is N or C—R^{y}, wherein R^{y} is —H or —F;
Z is —H, —F, —Cl, or C₁-C₂ alkyl;
R¹ is —H or C₁-C₃ alkyl,
   wherein the C₁-C₃ alkyl is optionally substituted by 1-4 substituents selected from the group consisting of —F, —OH, a 3- to 7-membered carbon-linked N-heterocycloalkyl, and —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl, and wherein R¹ may be cis or trans when R¹ is not —H;
n is an integer 0, 1, or 2;
each R² is independently —H or C₁-C₃ alkyl, wherein the C₁-C₃ alkyl is optionally substituted by 1-4 substituents selected from the group consisting of —F, —OH, a 3- to 7-membered carbon-linked N-heterocycloalkyl, and —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl; or
two R² groups on the same carbon atom may be taken together with the carbon atom to which they are attached to form a C₃-C₆ spirocycloalkyl or 4- to 6-membered spiroheterocycloalkyl containing 1-2 ring heteroatoms selected from the group consisting of N, O, and S; or
two R² groups on vicinal carbon atoms may be taken together with the two carbon atoms to form a fused C₃-C₆ cycloalkyl;
R³ is —H, —CD₃, C₁-C₃ alkyl, —CF₂H, —CN, —OR⁴, —SR⁴, —S(O)(C₁-C₃ alkyl), or —S(O)₂(C₁-C₃ alkyl);
each R⁴ is independently —H, C₁-C₃ alkyl, —CF₂H, —CF₃, or cyclopropyl;
R⁵ is C₁-C₃ alkyl, —CD₃, —CF₂H, allyl, —CH₂-cyclopropyl, cyclopropyl, or —OR⁴;
R⁶ is —H or —F;
R⁷ is —H or —F, and
R⁸ is —H or F.

In another aspect, provided herein is a compound of formula (IV)

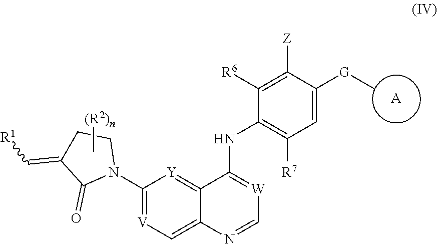

or pharmaceutically acceptable salt, solvate, hydrate, or co-crystal thereof, or a mixture of any of the foregoing, wherein:
ring A is

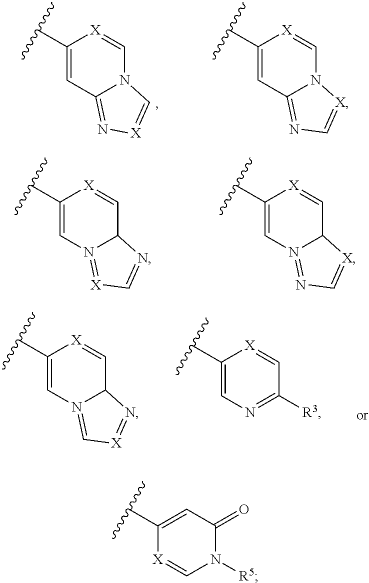

V is N or C—C⁸;
N is independently N or CN;
G is —CR^{g1}R^{g2}—, —O—, or —S—, wherein R^{g1} and R^{g2} are independently —H or —F;
Y is N or C—R^{y}, wherein R^{y} is —H or —F;
Z is —H, —F, —Cl, or C₁-C₂ alkyl;
R¹ is —H or C₁-C₃ alkyl,
   wherein the C₁-C₃ alkyl is optionally substituted by 1-4 substituents selected from the group consisting of —F, —OH, a 3- to 7-membered carbon-linked N-heterocycloalkyl, and —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl, and wherein R¹ may be cis or trans when R¹ is not —H;
n is an integer 0, 1, or 2;
each R² is independently —H or C₁-C₃ alkyl, wherein the C₁-C₃ alkyl is optionally substituted by 1-4 substituents selected from the group consisting of —F, —OH, a 3- to 7-membered carbon-linked N-heterocycloalkyl, and —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl; or
two R² groups on the same carbon atom may be taken together with the carbon atom to which they are attached to form a C₃-C₆ spirocycloalkyl or 4- to 6-membered spiroheterocycloalkyl containing 1-2 ring heteroatoms selected from the group consisting of N, O, and S; or
two R² groups on vicinal carbon atoms may be taken together with the two carbon atoms to form a fused C₃-C₆ cycloalkyl;
R³ is —H, —CD₃, C₁-C₃ alkyl, —CF₂H, —CN, —OR⁴, —SR⁴, —S(O)(C₁-C₃ alkyl), or —S(O)₂(C₁-C₃ alkyl);
R⁴ is —H, C₁-C₃ alkyl, —CF₂H, —CF₃, or cyclopropyl;
R⁵ is C₁-C₃ alkyl, —CD₃, —CF₂H, allyl, —CH₂-cyclopropyl, cyclopropyl, or —OR⁴;
R⁶ is —H or —F;
R⁷ is —H or —F, and
R⁸ is —H or F.

In some embodiments, ring A is

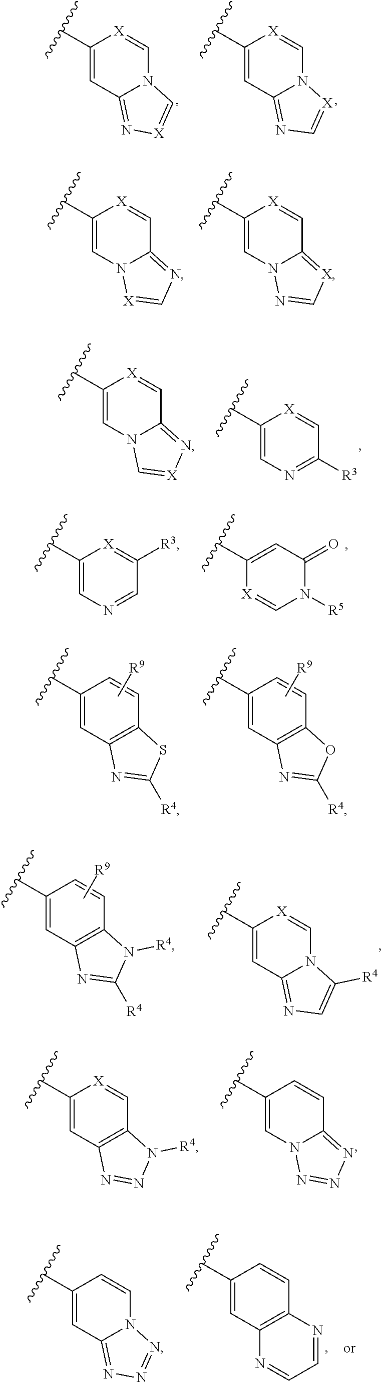

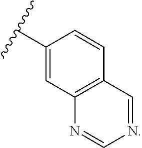 [SMILES: *c1ccc2cncnc2c1]

In some embodiments, ring A is

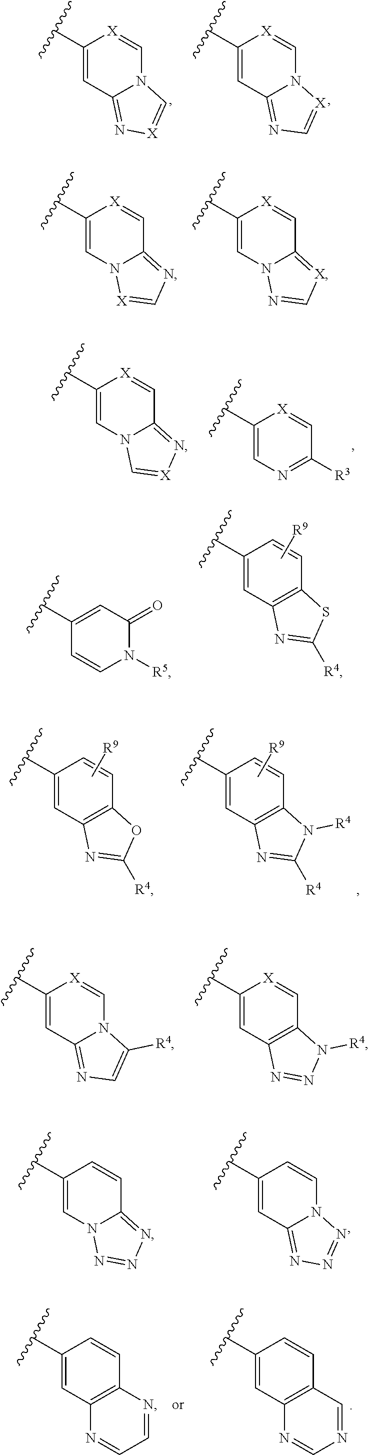

In some embodiments, ring A is

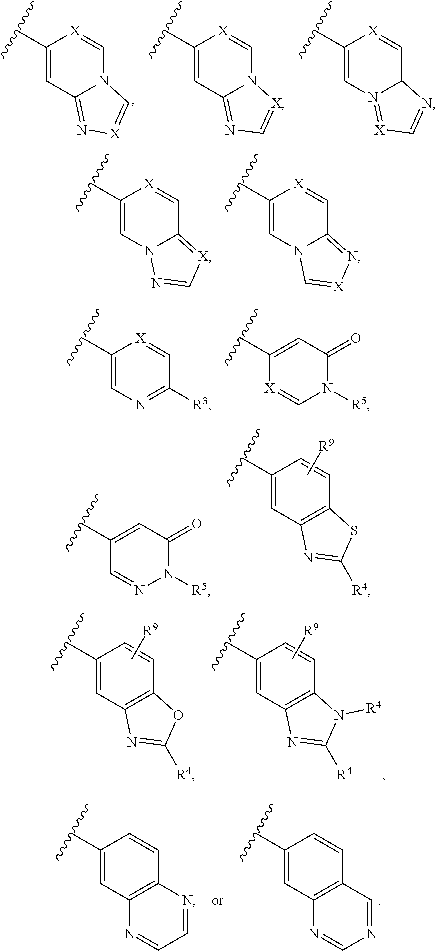

In some embodiments, ring A is

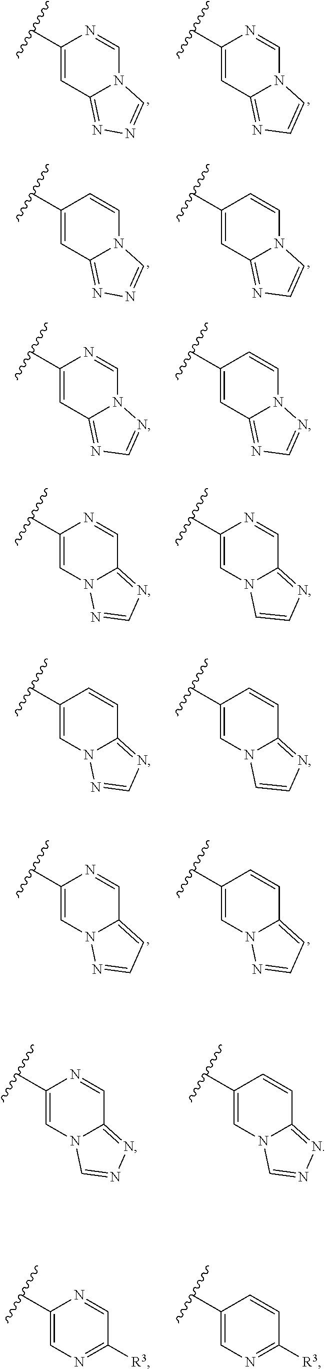

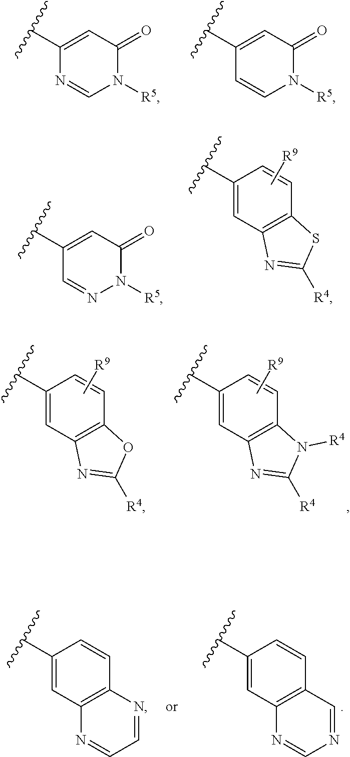

In some embodiments, ring A is

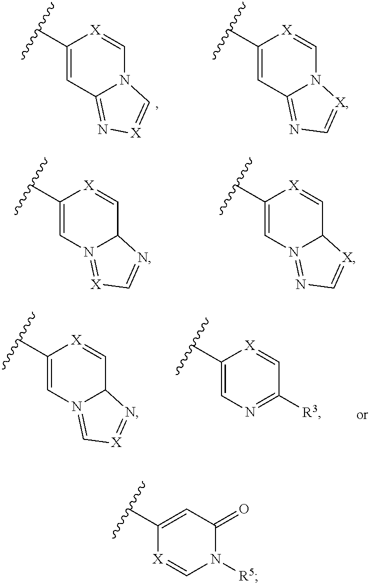

In some embodiments, ring A is

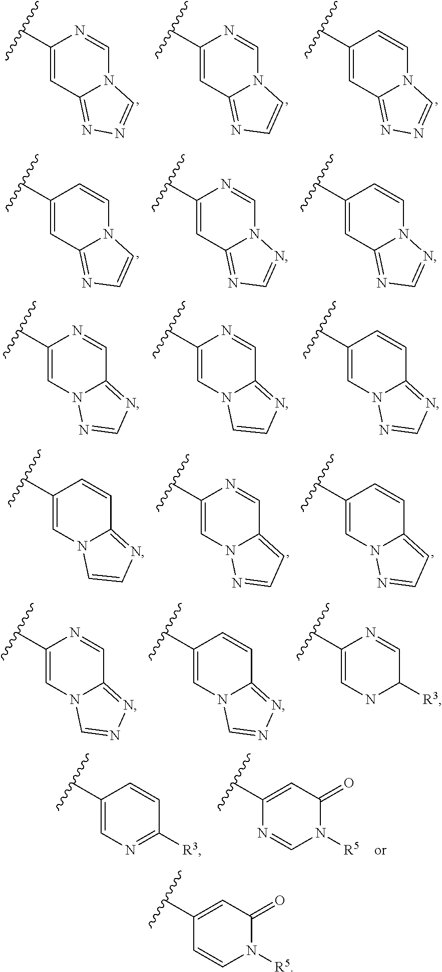

In some embodiments, ring A is

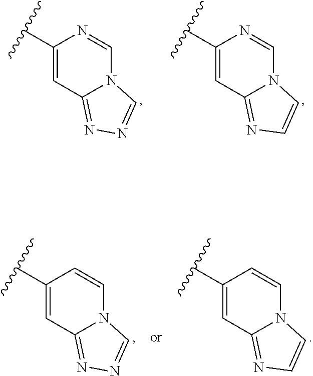 [SMILES: *c1cc2nncn2cn1 ; *c1cc2nccn2cn1 ; *c1ccn2cnnc2c1 ; *c1ccn2ccnc2c1]

In some embodiments, ring A is

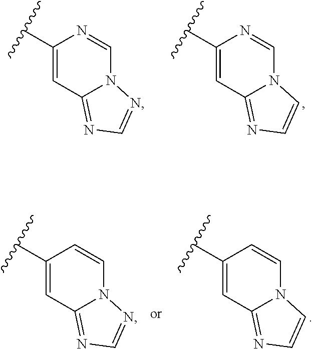 [SMILES: *c1cc2ncnn2cn1 ; *c1cc2nccn2cn1 ; *c1ccn2ncnc2c1 ; *c1ccn2ccnc2c1 ; "."]

In some embodiments, ring A is,

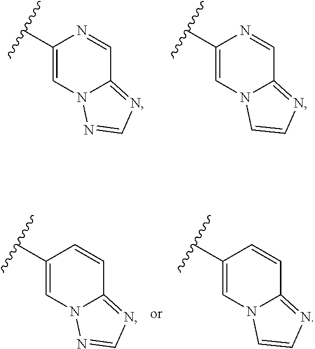 [SMILES: *c1cn2ncnc2cn1 ; *c1cn2ccnc2cn1 ; *c1ccc2ncnn2c1 ; *c1ccc2nccn2c1]

In some embodiments, ring A is

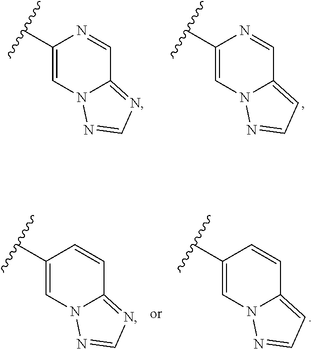 [SMILES: *c1cn2ncnc2cn1 ; *c1cn2nccc2cn1 ; "," ; *c1ccc2ncnn2c1 ; *c1ccc2ccnn2c1 ; "."]

In some embodiments, ring A is

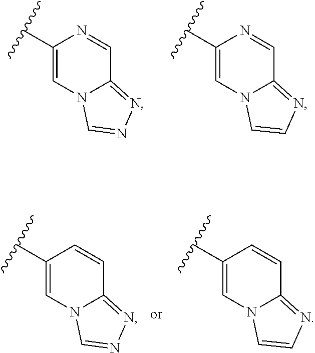 [SMILES: *c1cn2cnnc2cn1 ; *c1cn2ccnc2cn1 ; *c1ccc2nncn2c1 ; *c1ccc2nccn2c1]

In some embodiments, ring A is

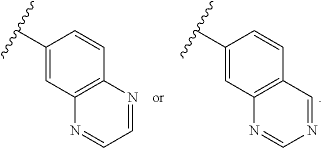 [SMILES: *c1ccc2nccnc2c1 ; *c1ccc2cncnc2c1]

In some embodiments, ring A is

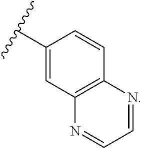 [SMILES: *c1ccc2nccnc2c1]

In some embodiments, ring A is

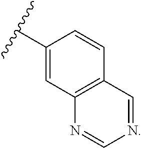 [SMILES: *c1ccc2cncnc2c1]

In some embodiments, ring A is

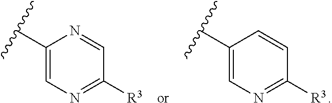

In some embodiments, ring A is

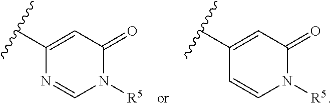

In some embodiments, ring A is

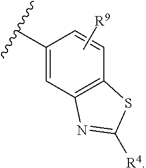

In some embodiments ring A is

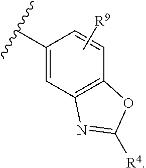

In some embodiments, ring A is

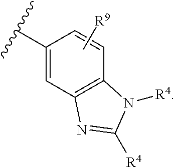

In some embodiments, ring A is

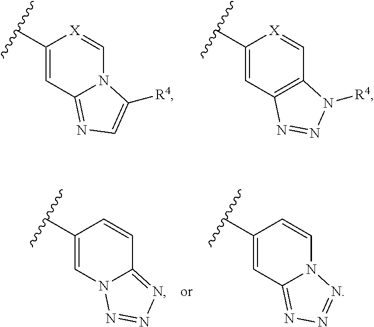

In some embodiments, ring A is

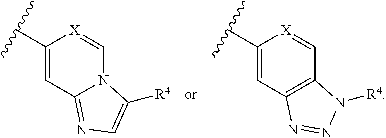

In some embodiments, ring A is

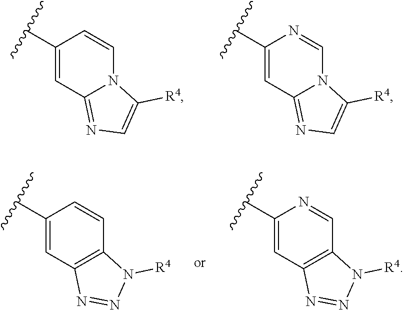

In some embodiments, ring A is

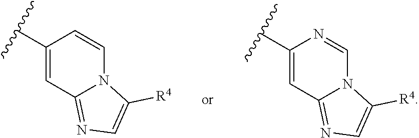

In some embodiments, ring A is

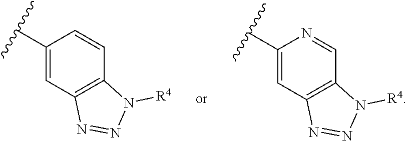

In some embodiments, ring A is

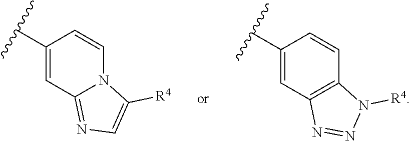

In some embodiments, ring A is

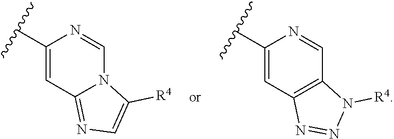

In some embodiments, ring A is

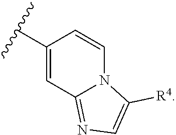

In some embodiments, ring A is

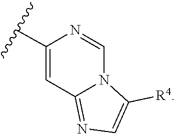

In some embodiments, ring A is

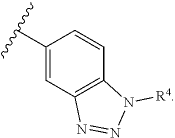

In some embodiments, ring A is

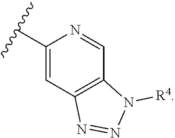

In some embodiments, ring A is

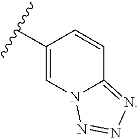 [SMILES: *c1ccc2nnnn2c1]

In some embodiments, ring A is

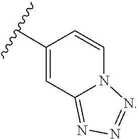 [SMILES: *c1ccn2nnnc2c1]

In some embodiments, ring A is

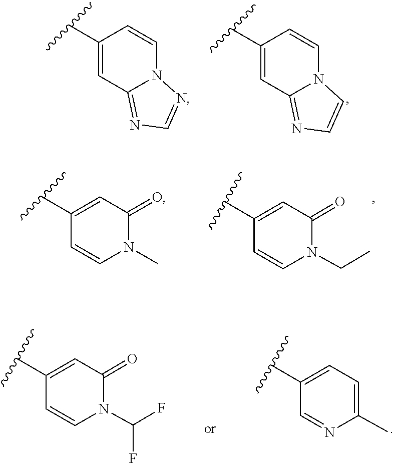

In some embodiments, ring A is

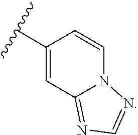 [SMILES: *c1ccn2ncnc2c1]

In some embodiments, ring A is

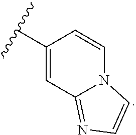 [SMILES: *c1ccn2ccnc2c1]

In some embodiments, ring A is

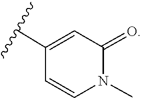 [SMILES: *c1ccn(C)c(=O)c1]

In some embodiments, ring A is

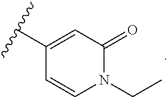 [SMILES: *c1ccn(CC)c(=O)c1]

In some embodiments, ring A is

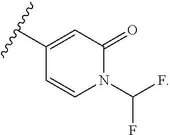 [SMILES: *c1ccn(C(F)F)c(=O)c1]

In some embodiments, ring A is

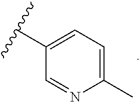 [SMILES: *c1ccc(C)nc1]

In some embodiments, ring A is

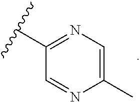 [SMILES: *c1cnc(C)cn1]

In some embodiments, ring A is

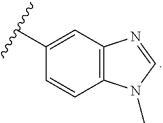 [SMILES: *c1ccc2c(c1)ncn2C]

In some embodiments, ring A is

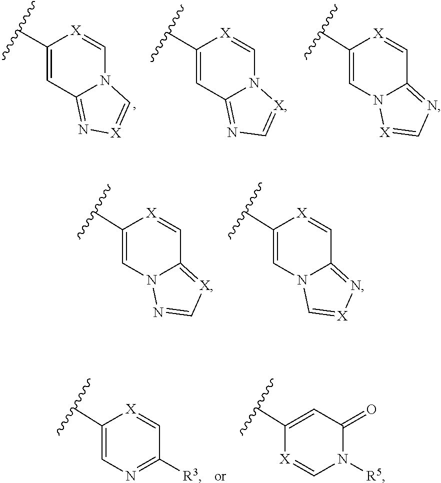

and each X is independently N or CH.
In some embodiments, ring A is

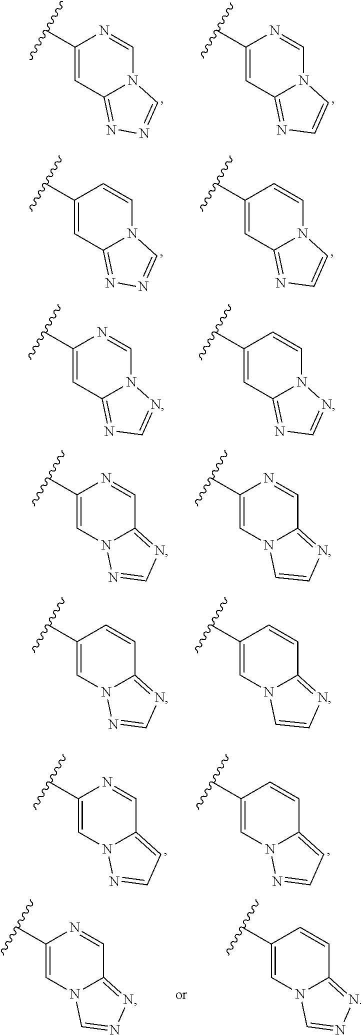

In some embodiments, ring A is

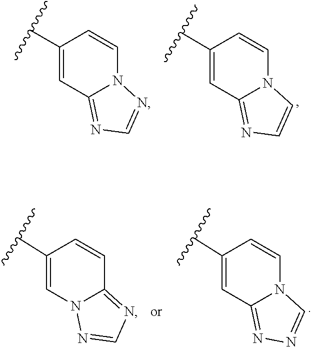 [SMILES: *c1ccn2ncnc2c1 ; *c1ccn2ccnc2c1 ; *c1ccc2ncnn2c1 ; *c1ccn2cnnc2c1]

In some embodiments, ring A is

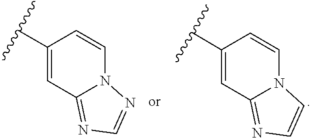 [SMILES: *c1ccn2ncnc2c1 ; *c1ccn2ccnc2c1]

In some embodiments, ring A is

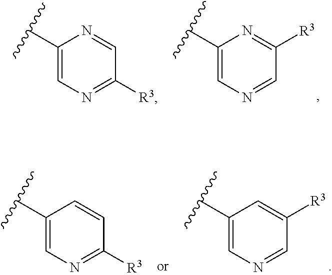

In some embodiments, ring A is

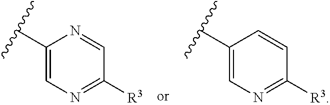

In some embodiments, ring A is

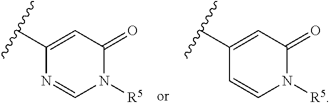

In some embodiments, ring A is

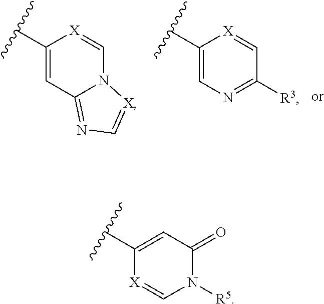

In some embodiments, ring A is

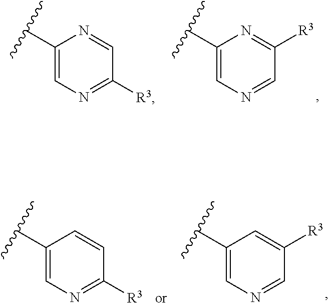

and R³ is —H, —CD₃, C₁-C₃ alkyl, —CF₂H, —CN, —OR⁴, —SR⁴, —S(O)(C₁-C₃ alkyl), or —S(O)₂(C₁-C₃ alkyl). In some embodiments, rign A is

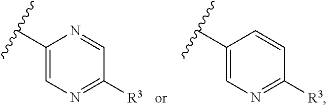

and R³ is —H, —CD₃, C₁-C₃ alkyl, CF₂H, —CN, —OR⁴, —SR⁴, —S(O)(C₁-C₃ alkyl), or S(O)₂(C₁-C₃ alkyl). In some embodiments, R³ is —H, —CD₃, C₁-C₂ alkyl, —CF₂H, —CN, —OR⁴, —SR⁴, —S(O)(C₁-C₂ alkyl), or —S(O)₂(C₁-C₂ alkyl). In some embodiments, R³ is —H, —CD₃, —CH₃, —CF₂H, —CN, —OR⁴, —SR⁴, —S(O)(CH₃), or —S(O)₂(CH₃). In some embodiments, R³ is —CH₃ or —CD₃. In some embodiments, R³ is C₁-C₃ alkyl. In some embodiments, R³ is methyl, ethyl, n-propyl, or isopropyl. In some embodiments, R³ is —S(O)(methyl), —S(O)(ethyl), —S(O)(n-propyl), or —S(O)(isopropyl). In some embodiments, R³ is —S(O)₂(methyl), —S(O)₂(ethyl), —S(O)₂(n-propyl), or —S(O)₂(isopropyl). In some embodiments, ring A is

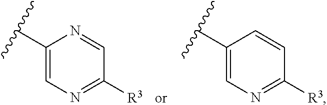

R³ is —H, —CD₃, C₁-C₂ alkyl, or —CF₂H. In some embodiments, ring A is

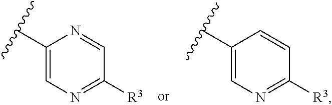

R³ is —CH₃ or —CH₂CH₃.

In some embodiments, ring A is

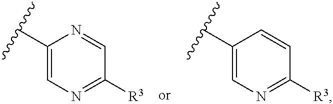

R³ is —OR⁴ or —SR⁴, and R⁴ is —H, —CD₃, C₁-C₃ alkyl, —CF₂H, —CF₃, or cyclopropyl. In some embodiments, ring A is

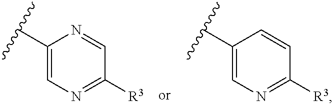

R³ is —OR⁴ or —SR⁴, and R⁴ is —H, C₁-C₃ alkyl, —CF₂H, —CF₃, or cyclopropyl. In some embodiments, R³ is —OR⁴ or —SR⁴, and R⁴ is —H, C₁-C₂ alkyl, —CF₂H, —CF₃, or cyclopropyl. In some embodiments, R³ is —OR⁴ or —SR⁴, and R⁴ is —H, —CH₃, —CHF₂, or cyclopropyl. In some embodiments, R³ is —OH or —SH. In some embodiments, R³ is —O(C₁-C₃ alkyl) or —S(C₁-C₃ alkyl). In some embodiments, R³ is —O(methyl), —O(ethyl), —O(n-propyl), —O(isopropyl), —S(methyl), —S(ethyl), —S(n-propyl), or —S(isopropyl). In some embodiments, R³ is —O(cyclopropyl). In some embodiments, R³ is —O(CF₂H) or —S(CF₂H). In some embodiments, R³ is —O(CF₃) or —S(CF₃).

In some embodiments, ring A is

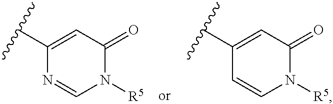

and R⁵ is C₁-C₃ alkyl, —CD₃, —CF₂H, allyl, —CH₂-cyclopropyl, cyclopropyl, or —OR⁴. In some embodiments, R⁵ is C₁-C₂ alkyl, —CD₃, —CF₂H, allyl, —CH₂-cyclopropyl, cyclopropyl, or —OR⁴. In some embodiments, R⁵ is —CH₃ or —CD₃. In some embodiments, R⁵ is C₁-C₃ alkyl. In some embodiments, R⁵ is methyl, ethyl, n-propyl, or isopropyl. In some embodiments, R⁵ is cyclopropyl. In some embodiments, ring A is

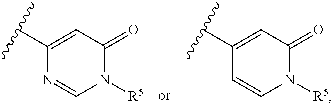

and R⁵ is C₁-C₂ alkyl, —CD₃, or —CF₂H. In some embodiments, ring A is

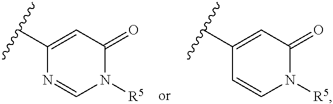

and R⁵ is —CH₃ or —CF₂H.

In some embodiments, ring A is

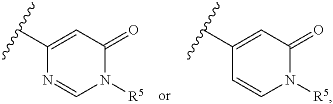

R⁵ is —OR⁴, and R⁴ is —H, —CD₃, C₁-C₃ alkyl, —CF₂H, —CH₃, or cyclopropyl. In some embodiments, ring A is

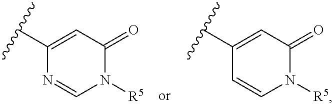

R⁵ is —OR⁴, and R⁴ is —H, C₁-C₃ alkyl, —CF₂H, —CF₃, or cyclopropyl. In some embodiments, R⁵ is —OR⁴, and R⁴ is —H, C₁-C₂ alkyl, —CF₂H, —CF₃, or cyclopropyl. In some embodiments, R⁵ is —OR⁴, and R⁴ is —H, —CH₃, —CHF₂, or cyclopropyl. In some embodiments, R⁵ is —OH. In some embodiments, R⁵ is —O(C₁-C₃ alkyl). In some embodiments, R⁵ is —O(methyl), —O(ethyl), —O(n-propyl), or —O(isopropyl). In some embodiments, R⁵ is —O(cyclopropyl). In some embodiments, R⁵ is —O(CF₂H). In some embodiments, R⁵ is —O(CF₃).

In some embodiments, ring A is

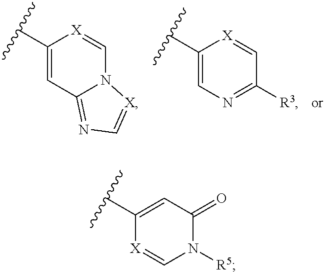

R³ is H, —CD₃, C₁-C₂ alkyl, or —CF₂H, and R⁵ is C₁-C₂ alkyl, —CD₃, or —CF₂H. In some embodiments, ring A is

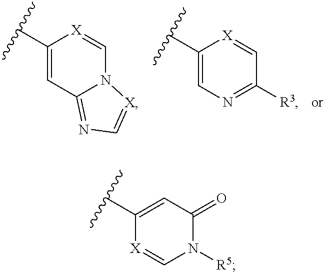

R³ is —CH₃ or —CF₂H, and R⁵ is —CH₃ or —CF₂H.

In some embodiments, ring A is

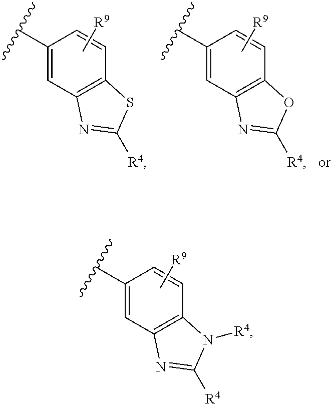

and each R⁴ is independently —H, —CD₃, C₁-C₃ alkyl, —CF₂H, —CF₃, or cyclopropyl. In some embodiments, ring A is

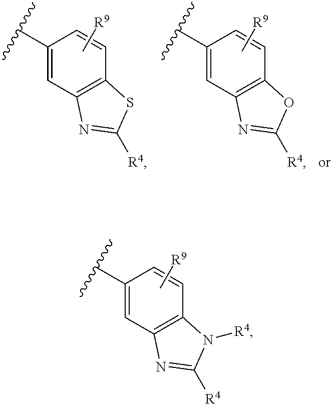

and each R⁴ is independently H, C₁-C₃ alkyl, —CF₂H, —CF₃, or cyclopropyl. In some embodiments, each R⁴ is independently H, C₁-C₂ alkyl, —CF₂H, —CF₃, or cyclopropyl. In some embodiments, each R⁴ is independently H, —CH₃, —CHF₂, or cyclopropyl. In some embodiments, each R⁴ is independently H. In some embodiments, each R⁴ is independently (C₁-C₃ alkyl). In some embodiments, each R⁴ is independently methyl, ethyl, -n-propyl, or -isopropyl. In some embodiments, R⁴ is cyclopropyl. In some embodiments, R⁴ is CF₂H. In some embodiments, R⁴ is CF₃.

In some embodiments, ring A is

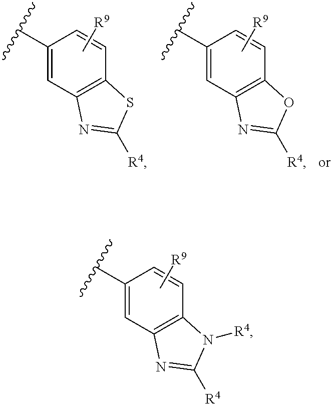

and R⁹ is H, halogen, C₁-C₃ alkyl, —CF₂H, —CF₃, cyclopropyl, —CN or —OR⁴. In some embodiments, R⁹ is H, halogen C₁-C₂ alkyl CF₂H, —CF₃, cyclopropyl, —CN or —OR⁴. In some embodiments, R⁹ is H. In some embodiments, R⁹ is halogen. In some embodiments, R⁹ is C₁-C₃ alkyl. In some embodiments, R⁹ is methyl, ethyl, n-propyl, or isopropyl. In some embodiments, R⁹ is cyclopropyl. In some embodiments, R⁹ is —CN. In some embodiments, R⁹ is —OR⁴.

In some embodiments, ring A is

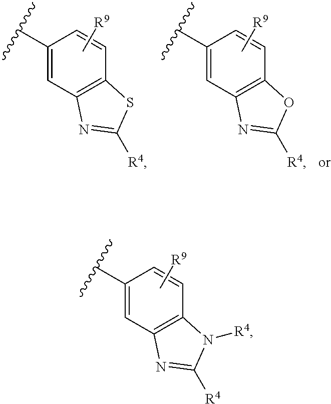

and R⁹ is —H, halogen, —CN, C₁-C₃ alkyl, —CF₂H, —CF₃, cyclopropyl, —O(C₁-C₃ alkyl), or —O— cyclopropyl, and each R⁴ is independently —H, —CD₃, C₁-C₃ alkyl, —CF₂H, —CF₃, or cyclopropyl. In some embodiments, ring A is

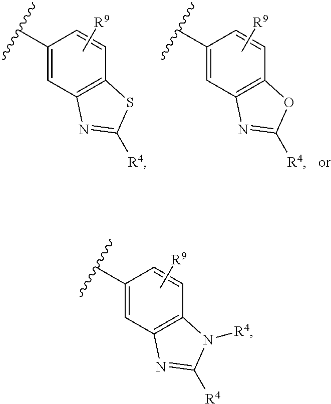

R⁹ is —OR⁴, and each R⁴ is independently H, C₁-C₃ alkyl, —CF₂H, —CF₃, or cyclopropyl. In some embodiments, R⁹ is —OR⁴, and each R⁴ is independently H, C₁-C₂ alkyl, —CF₂H, —CF₃, or cyclopropyl. In some embodiments, R⁹ is —OR⁴, and each R⁴ is independently H, —CH₃, —CHF₂, or cyclopropyl. In some embodiments, R⁹ is —OH. In some embodiments, R⁹ is —O(C₁-C₃ alkyl). In some embodiments, R⁹ is —O(methyl), —O(ethyl), —O(n-propyl), or —O(isopropyl). In some embodiments, R⁹ is —O(cyclopropyl). In some embodiments, R⁹ is —O(CF₂H). In some embodiments, R⁹ is —O(CF₃).

In some embodiments, ring A is

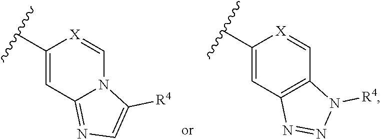

and R⁴ is independently —H, —CD₃, C₁-C₃ alkyl, —CF₂H, —CF₃, or cyclopropyl. In some embodiments, ring A is

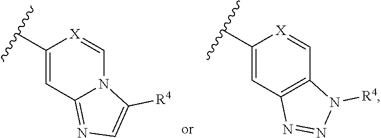

and each R⁴ is independently H, C₁-C₃ alkyl, —CF₂H, —CF₃, or cyclopropyl. In some embodiments, each R⁴ is independently H, C₁-C₂ alkyl, —CF₂H, —CF₃, or cyclopropyl. In some embodiments, each R⁴ is independently H, —CH₃, —CHF₂, or cyclopropyl. In some embodiments, each R⁴ is independently H. In some embodiments, each R⁴ is independently (C₁-C₃ alkyl). In some embodiments, each R⁴ is independently methyl, ethyl, -n-propyl, or -isopropyl. In some embodiments, R⁴ is cyclopropyl. In some embodiments, R⁴ is CF₂H. In some embodiments, R⁴ is CF₃. In some embodiments, ring A is

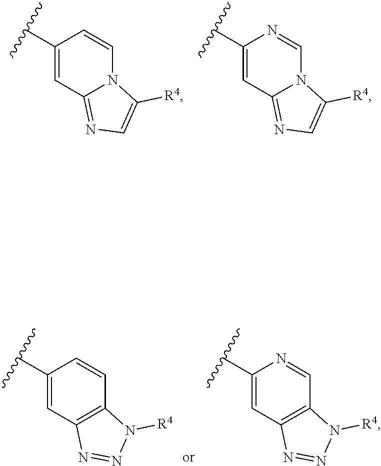

and each R⁴ is independently H, C₁-C₃ alkyl, —CF₂H, —CF₃, or cyclopropyl. In some embodiments, ring A is

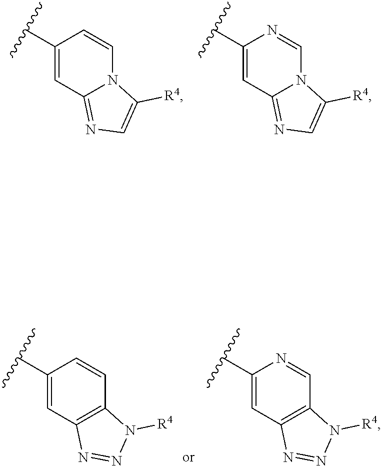

and R⁴ is independently —H, C₁-C₃ alkyl, —CF₂H, —CF₃, or cyclopropyl. In some embodiments, each R⁴ is independently H, C₁-C₂ alkyl, —CF₂H, —CF₃, or cyclopropyl. In some embodiments, each R⁴ is independently H, —CH₃, —CHF₂, or cyclopropyl. In some embodiments, each R⁴ is independently H. In some embodiments, each R⁴ is independently (C₁-C₃ alkyl). In some embodiments, each R⁴ is independently methyl, ethyl, -n-propyl, or -isopropyl. In some embodiments, R⁴ is cyclopropyl. In some embodiments, R⁴ is CF₂H. In some embodiments, R⁴ is CF₃. In some embodiments, ring A is

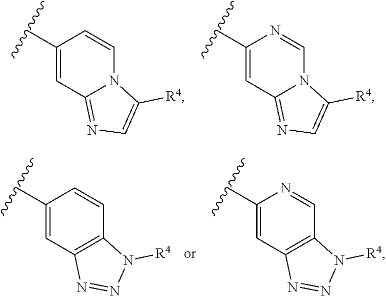

and each R⁴ is independently H, —CH₃, —CHF₂, or cyclopropyl.

In some embodiments, m is an integer 0 or 1, wherein if m=0, then V is S, and if m=1, then V is N or C—R⁸. In some embodiments, m is 0 and V is S. In other embodiments m is 1 and V is N or C—R⁸. In some embodiments wherein m is 1, V is N. In other embodiments wherein m is 1 and V is C—R⁸. In some embodiments, V is N. In other embodiments, V is C—R⁸.

In some embodiments, W is N. In other embodiments, W is C—CN.

In some embodiments, G is —CR^{g1}R^{g2}—, —O—, or —S—, wherein R^{g1} and R^{g2} are independently —H or —F. In some embodiments, G is —CH₂—, —CHF—, or —CF₂—. In some embodiments, G is —CH₂— or —O—. In some embodiments, G is —CH₂— or —S—. In some embodiments, G is —CR^{g1}R^{g2}— or —O—. In some embodiments, G is —CR^{g1}R^{g2}— or —S—. In some embodiments, G is —O— or —S—. In some embodiments, G is —CH₂—. In some embodiments, G is —CHF—. In some embodiments, G is —CF₂—. In some embodiments, G is —O—. In some embodiments, G is —S—.

In some embodiments, Y is N or C—R^{y}, wherein R^{y} is —H or —F. In some embodiments, Y is N. In some embodiments, Y is C—R^{y}, wherein R^{y} is —H or —F. In some embodiments, Y is C R^{y}, wherein R^{y} is —H. In some embodiments, Y is C—R^{y}, wherein R^{y} is —F.

In other embodiments, m is 1, V is N or C—R⁸, and the compound of formula (I) is a compound of formula (II-a), formula (II-b), formula (III), or formula (IV). In some embodiments, V is N, W is N, and Y is N. In some embodiments, V is C—R⁸, W is C—CN, and Y is C—R^{y}. In some embodiments, V is C—R⁸, W is N, and Y is N. In some embodiments, V is N, W is C—CN, and Y is N. In some embodiments, V is N, W is N and Y is C—R^{y}. In some embodiments, V is C—R⁸, W is C—CN, and Y is N. In some embodiments, V is N, W is C—CN, and Y is C—R^{y}. In some embodiments, V is C—R⁸, W is N, and Y is C—R^{y}.

In some embodiments, m is 0, V is S, and the compound of formula (I) is a compound of formula (II-c) or formula (II-d). In some embodiments, V is S, W is N, and Y is N. In some embodiments, V is S, W is N, and Y is C—R^{y}. In some embodiments, V is S, W is C—CN, and Y is N. In some embodiments, V is S, W is C—CN, and Y is C—R^{y}. In some embodiments, V is S, W is N, and Y is N. In some embodiments, V is S, W is N and Y is C—R^{y}.

In some embodiments, Z is —H, halogen, —OCH₃, —C≡CH, or C₁-C₂ alkyl. In some embodiments, Z is halogen. In some embodiments, Z is —OCH₃. In Some embodiments, Z is —C≡CH. In some embodiments, Z is C₁-C₂ alkyl. In some embodiments, Z is —H, —F, —Cl, or C₁-C₂ alkyl. In some embodiments, Z is —H, —F, —Cl, or —CH₃. In some embodiments, Z is —CH₃. In some embodiments, Z is —H. In some embodiments, Z is —F. In some embodiments, Z is —Cl. In some embodiments, Z is —CH₂CH₃.

In some embodiments, R¹ is —H, C₁-C₃ alkyl, or 3- to 7-membered heterocycloalkyl, wherein the C₁-C₃ alkyl is optionally substituted by 1-4 substituents selected from the group consisting of —F, —OH, a 3- to 7-membered carbon-linked N-heterocycloalkyl, and —NR^{1a}R^{1b} wherein each R^{1a} and R^{1b} is independently hydrogen, —CD₃, or C₁-C₃ alkyl, or wherein each pair of geminal R^{1a} and R^{1b} may be taken together with the nitrogen atom to which they are attached to form an N-heterocycloalkyl, wherein the 3- to 7-membered heterocycloalkyl of R¹ is optionally substituted by —F, and wherein each heterocyclic nitrogen atom, if present, is independently optionally substituted with C₁-C₃ alkyl, and wherein R¹ may be cis or trans when R¹ is not —H. In some embodiments, R¹ is —H, C₁-C₃ alkyl, or 3- to 7-membered heterocycloalkyl, wherein the C₁-C₃ alkyl is optionally substituted by 1-4 substituents selected from the group consisting of —F, —OH, a 3- to 7-membered carbon-linked N-heterocycloalkyl, and —NR^{1a}R^{1b} wherein each R^{1a} and R^{1b} is independently hydrogen, —CD₃, or C₁-C₃ alkyl, or wherein each pair of geminal R^{1a} and R^{1b} may be taken together with the nitrogen atom to which they are attached to form an N-heterocycloalkyl, and wherein each heterocyclic nitrogen atom, if present, is independently optionally substituted with C₁-C₃ alkyl, and wherein R¹ may be cis or trans when R¹ is not —H. In some embodiments, R¹ is —H or C₁-C₃ alkyl, wherein the C₁-C₃ alkyl is optionally substituted by 1-4 substituents selected from the group consisting of —F, —OH, a 3- to 7-membered carbon-linked N-heterocycloalkyl, and —NR^{1a}R^{1b} wherein each R^{1a} and R^{1b} are independently hydrogen, —CD₃, or C₁-C₃ alkyl, or wherein each pair of geminal R^{1a} and R^{1b} may be taken together with the nitrogen atom to which they are attached to form an N-heterocycloalkyl, and wherein R¹ may be cis or trans when R¹ is not —H.

In some embodiments, R¹ is 3- to 7-membered heterocycloalkyl, wherein the 3- to 7-membered heterocycloalkyl is optionally substituted by —F, wherein each heterocyclic nitrogen atom, if present, is independently optionally substituted with C₁-C₃ alkyl, and wherein R¹ may be cis or trans when R¹ is not —H. In some embodiments, R¹ is 3- to 7-membered heterocycloalkyl wherein each heterocyclic nitrogen atom, if present, is independently optionally substituted with C₁-C₃ alkyl, and wherein R¹ may be cis or trans when R¹ is not —H. In some embodiments, R¹ is 3- to 7-membered heterocycloalkyl containing at least 1 nitrogen atom. In other embodiments, R¹ is 3- to 7-membered heterocycloalkyl containing at least 2 nitrogen atoms. In some embodiments, R¹ is nitrogen-linked 3- to 7-membered heterocycloalkyl. In certain embodiments, R¹ is nitrogen-linked 4- to 6-membered heterocycloalkyl. In other embodiments, R¹ is carbon-linked 3- to 7-membered heterocycloalkyl. In certain other embodiments, R¹ is carbon-linked 4- to 6-membered heterocycloalkyl. In some embodiments wherein R¹ is a carbon-linked 3- to 7-membered heterocycloalkyl containing at least one nitrogen atom or R¹ is a nitrogen-linked 3- to 7-membered heterocycloalkyl containing at least two nitrogen atoms, each heterocyclic nitrogen, if present and valency permits, is substituted with C₁-C₃ alkyl.

In some embodiments, R¹ is —H or C₁-C₃ alkyl, wherein the C₁-C₃ alkyl is optionally substituted by 1-4 substituents selected from the group consisting of —F, —OH, a 3- to 7-membered carbon-linked N-heterocycloalkyl, and —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl, and wherein R¹ may be cis or trans when R¹ is not —H. In some embodiments, R¹ is —H. In some embodiments, R¹ is C₁-C₃ alkyl. In some embodiments, R¹ is optionally substituted C₁-C₃ alkyl. In some embodiments, R¹ is unsubstituted C₁-C₃ alkyl. In some embodiments, R¹ is methyl, ethyl, n-propyl, or isopropyl, each of which is unsubstituted. In some embodiments, R¹ is —CH₃.

In some embodiments, R¹ is methyl, ethyl, n-propyl, or isopropyl, each of which is substituted by 1-3 F groups. In some embodiments, R¹ is —CH₂F, —CHF₂, —CF₃, —CH₂CH₂F, —CH₂CHF₂, —CH₂CF₃, —CHFCH₃, —CF₂CH₃, —CHFCH₂F, —CF₂CH₂F, —CHFCHF₂, —CH₂CH₂CH₂F, —CH₂CH₂CHF₂, —CH₂CH₂CF₃, —CH₂CHFCH₂F, —CH₂CHFCHF₂, —CH₂CF₂CH₂F, —CHFCHFCH₂F, —CF₂CH₂CH₂F, —CHFCH₂CH₃, —CH₂CHFCH₃, —CHFCHFCH₃, —CHFCF₂CH₃, or —CF₂CHFCH₃.

In some embodiments, R¹ is methyl, ethyl, n-propyl, or isopropyl, each of which is substituted by 1-3 —OH groups. In some embodiments, R¹ is methyl-OH, ethyl-OH, n-propyl-OH, or isopropyl-OH. In some embodiments, R¹ is —CH₂OH, —CH(OH)CH₃, —CH₂CH₂OH, —CH(OH)CH₂CH₃, —CH₂CH(OH)CH₃, —CH₂CH₂CH₂OH, —C(CH₃)₂OH, or —CH(CH₂OH)(CH₃).

In some embodiments, R¹ is methyl, ethyl, n-propyl, or isopropyl, each of which is substituted by 3- to 7-membered carbon-linked N-heterocycloalkyl. In some embodiments, R¹ is methyl, ethyl, n-propyl, or isopropyl, each of which is substituted by 4- to 6-membered carbon-linked N-heterocycloalkyl. In some embodiments, R¹ is methyl, ethyl, n-propyl, or isopropyl, each of which is substituted by 3- to 7-membered carbon-linked N-heterocycloalkyl containing 1 nitrogen atom. In some embodiments, R¹ is methyl, ethyl, n-propyl, or isopropyl, each of which is substituted by 3- to 7-membered carbon-linked N-heterocycloalkyl containing 2 nitrogen atoms. In some embodiments, R¹ is methyl, ethyl, n-propyl, or isopropyl, each of which is substituted by 4- to 6-membered carbon-linked heterocycloalkyl containing 1 nitrogen atom. In some embodiments, R¹ is methyl, ethyl, n-propyl, or isopropyl, each of which is substituted by 4- to 6-membered carbon-linked heterocycloalkyl containing 2 nitrogen atoms. In some embodiments, R¹ is methyl, ethyl, n-propyl, or isopropyl, each of which is substituted by carbon-linked azetidinyl, diazetidinyl, pyrrolidinyl, pyrazolidinyl, imidazolidinyl, piperidinyl, or piperazinyl. In some embodiments, R¹ is methyl, ethyl, n-propyl, or isopropyl, each of which is substituted by carbon-linked azetidinyl, pyrrolidinyl, or piperidinyl.

In some embodiments, R¹ is methyl, ethyl, n-propyl, or isopropyl, each of which is substituted by —NWT (wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl.

In some embodiments, R¹ is methyl, ethyl, n-propyl, or isopropyl, each of which is substituted by —NWT (wherein each R^{1a} and R^{1b} are independently C₁-C₃ alkyl. In some embodiments, R¹ is methyl, ethyl, n-propyl, or isopropyl, each of which is substituted by —N(C₁-C₃ alkyl)(C₁-C₃ alkyl). In some embodiments, R¹ is methyl, ethyl, n-propyl, or isopropyl, each of which is substituted by —N(methyl)(methyl), —N(methyl)(ethyl), —N(methyl)(n-propyl), —N(methyl)(isopropyl), —N(ethyl)(ethyl), —N(ethyl)(n-propyl), —N(ethyl)(isopropyl), —N(n-propyl)(n-propyl), —N(n-propyl)(isopropyl), or —N(isopropyl)(isopropyl). In some embodiments, R¹ is methyl substituted by —N(methyl)(methyl), —N(methyl)(ethyl), —N(methyl)(n-propyl), —N(methyl)(isopropyl), —N(ethyl)(ethyl), —N(ethyl)(n-propyl), —N(ethyl)(isopropyl), —N(n-propyl)(n-propyl), —N(n-propyl)(isopropyl), or —N(isopropyl)(isopropyl). In some embodiments, R¹ is methyl, ethyl, n-propyl, or isopropyl, each of which is substituted by —N(methyl)(methyl). In some embodiments, R¹ is methyl substituted by —N(methyl)(methyl).

In some embodiments, R¹ is methyl, ethyl, n-propyl, or isopropyl, each of which is substituted by —NR^{1a}R^{1b} wherein each pair of geminal R^{1a} and R^{1b} may be taken together with the nitrogen atom to which they are attached to form an N-heterocycloalkyl. In some embodiments, each pair of geminal R^{1a} and R^{1b} may be taken together to form a 3- to 7-membered N-heterocycloalkyl. In some embodiments, R¹ is methyl, ethyl, n-propyl, or isopropyl, each of which is substituted by 3- to 7-membered nitrogen-linked N-heterocycloalkyl. In some embodiments, R¹ is methyl, ethyl, n-propyl, or isopropyl, each of which is substituted by 4- to 6-membered nitrogen-linked N-heterocycloalkyl. In some embodiments, R¹ is methyl, ethyl, n-propyl, or isopropyl, each of which is substituted by 3- to 7-membered nitrogen-linked N-heterocycloalkyl containing 1 nitrogen atom. In some embodiments, R¹ is methyl, ethyl, n-propyl, or isopropyl, each of which is substituted by 3- to 7-membered nitrogen-linked N-heterocycloalkyl containing 2 nitrogen atoms. In some embodiments, R¹ is methyl, ethyl, n-propyl, or isopropyl, each of which is substituted by 4- to 6-membered nitrogen-linked heterocycloalkyl containing 1 nitrogen atom. In some embodiments, R¹ is methyl, ethyl, n-propyl, or isopropyl, each of which is substituted by 4- to 6-membered nitrogen-linked heterocycloalkyl containing 2 nitrogen atoms. In some embodiments, R¹ is methyl, ethyl, n-propyl, or isopropyl, each of which is substituted by nitrogen-linked azetidinyl, diazetidinyl, pyrrolidinyl, pyrazolidinyl, imidazolidinyl, piperidinyl, or piperazinyl. In some embodiments, R¹ is methyl, ethyl, n-propyl, or isopropyl, each of which is substituted by nitrogen-linked azetidinyl, pyrrolidinyl, or piperidinyl.

In some embodiments wherein R¹ is not —H, R¹ is cis to the carbonyl moiety on the lactam ring. In some embodiments wherein R¹ is not —H, R¹ is trans to the carbonyl moiety on the lactam ring.

In some embodiments, n is an integer 0, 1, or 2. In some embodiments, n is an integer 1 or 2. In some embodiments, n is 0. In some embodiments, n is 1. In some embodiments, n is 2.

In some embodiments, each R² is independently —H or C₁-C₃ alkyl, wherein the C₁-C₃ alkyl is optionally substituted by 1-4 substituents selected from the group consisting of —F, —OH, —O(C₁-C₃ alkyl), a 3- to 7-membered carbon-linked N-heterocycloalkyl, and —NR^{1a}R^{1b} wherein the C₁-C₃ alkyl of the —O(C₁-C₃ alkyl) is optionally substituted by NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl.

In some embodiments, each R² is independently —H or C₁-C₃ alkyl, wherein the C₁-C₃ alkyl is optionally substituted by 1-4 substituents selected from the group consisting of —F, —OH, a 3- to 7-membered carbon-linked N-heterocycloalkyl, and —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl. In some embodiments wherein n is 2 and two R² groups are on the same carbon atom, the two R² groups on the same carbon atom may be taken together with the carbon atom to which they are attached to form a C₃-C₆ spirocycloalkyl or 4- to 6-membered spiroheterocycloalkyl containing 1-2 ring heteroatoms selected from the group consisting of N, O, and S. In other embodiments wherein n is 2 and two R² groups are on vicinal carbon atoms, the two R² groups on vicinal carbon atoms may be taken together with the two carbon atoms to form a fused C₃-C₆ cycloalkyl. In some embodiments, R² is —H or C₁-C₃ alkyl. In some embodiments, R² is —H. In some embodiments, R² is C₁-C₃ alkyl. In some embodiments, R² is methyl, ethyl, n-propyl, or isopropyl.

In some embodiments wherein R² is C₁-C₃ alkyl, the C₁-C₃ alkyl is optionally substituted by 1-4 substituents selected from the group consisting of —F, —OH, —O(C₁-C₃ alkyl), a 3- to 7-membered carbon-linked N-heterocycloalkyl, and NR^{1a}R^{1b} wherein the C₁-C₃ alkyl of the —O(C₁-C₃ alkyl) is optionally substituted by —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl. In some embodiments wherein R² is C₁-C₃ alkyl, the C₁-C₃ alkyl is optionally substituted by 1-4 substituents selected from the group consisting of —F, —OH, a 3- to 7-membered carbon-linked N-heterocycloalkyl, and —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl.

In some embodiments, R² is methyl, ethyl, n-propyl, or isopropyl, each of which is substituted by 1-3 F groups. In some embodiments, R² is —CH₂F, —CHF₂, —CF₃, —CH₂CH₂F, —CH₂CHF₂, —CH₂CF₃, —CHFCH₃, —CF₂CH₃, —CHFCH₂F, —CF₂CH₂F, —CHFCHF₂, —CH₂CH₂CH₂F, —CH₂CH₂CHF₂, —CH₂CH₂CF₃, —CH₂CHFCH₂F, —CH₂CHFCHF₂, —CH₂CF₂CH₂F, —CHFCHFCH₂F, —CF₂CH₂CH₂F, —CHFCH₂CH₃, —CH₂CHFCH₃, —CHFCHFCH₃, —CHFCF₂CH₃, or —CF₂CHFCH₃.

In some embodiments, R² is methyl, ethyl, n-propyl, or isopropyl, each of which is substituted by 1-3 —OH groups. In some embodiments, R² is methyl-OH, ethyl-OH, n-propyl-OH, or isopropyl-OH. In some embodiments, R² is —CH₂OH, —CH(OH)CH₃, —CH₂CH₂OH, —CH(OH)CH₂CH₃, —CH₂CH(OH)CH₃, —CH₂CH₂CH₂OH, —C(CH₃)₂OH, or —CH(CH₂OH)(CH₃).

In some embodiments, R² is methyl, ethyl, n-propyl, or isopropyl, each of which is substituted by 1-3 —O(C₁-C₃ alkyl), wherein each C₁-C₃ alkyl of each —O(C₁-C₃ alkyl) is optionally further substituted by —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl. In some embodiments, R² is methyl substituted by —OCH₃, —OCH₂CH₃, —O(CH₂)₂CH₃, —OCH₂NR^{1a}R^{1b}, —OCH₂CH₂NR^{1a}R^{1b}, or —O(CH₂)₃NR^{1a}R^{1b} In some embodiments, R² is ethyl substituted by —OCH₃, —OCH₂CH₃, —O(CH₂)₂CH₃, —OCH₂NR^{1a}R^{1b} —OCH₂CH₂NR^{1a}R^{1b} or —O(CH₂)₃NR^{1a}R^{1b} In some embodiments, R² is n-propyl substituted by —OCH₃, —OCH₂CH₃, —O(CH₂)₂CH₃, —OCH₂NR^{1a}R^{1b}, —O(CH₂)₂NR^{1a}R^{1b} or —O(CH₂)₃NR^{1a}R^{1b} In some embodiments, R² is methyl substituted by —O(CH₂)₂N(CH₃)₂.

In some embodiments, R² is methyl, ethyl, n-propyl, or isopropyl, each of which is substituted by 3- to 7-membered carbon-linked N-heterocycloalkyl. In some embodiments, R² is methyl, ethyl, n-propyl, or isopropyl, each of which is substituted by 4- to 6-membered carbon-linked N-heterocycloalkyl. In some embodiments, R² is methyl, ethyl, n-propyl, or isopropyl, each of which is substituted by 3- to 7-membered carbon-linked N-heterocycloalkyl containing 1 nitrogen atom. In some embodiments, R² is methyl, ethyl, n-propyl, or isopropyl, each of which is substituted by 3- to 7-membered carbon-linked N-heterocycloalkyl containing 2 nitrogen atoms. In some embodiments, R² is methyl, ethyl, n-propyl, or isopropyl, each of which is substituted by 4- to 6-membered carbon-linked heterocycloalkyl containing 1 nitrogen atom. In some embodiments, R² is methyl, ethyl, n-propyl, or isopropyl, each of which is substituted by 4- to 6-membered carbon-linked heterocycloalkyl containing 2 nitrogen atoms. In some embodiments, R² is methyl, ethyl, n-propyl, or isopropyl, each of which is substituted by carbon-linked azetidinyl, diazetidinyl, pyrrolidinyl, pyrazolidinyl, imidazolidinyl, piperidinyl, or piperazinyl. In some embodiments, R² is methyl, ethyl, n-propyl, or isopropyl, each of which is substituted by carbon-linked azetidinyl, pyrrolidinyl, or piperidinyl.

In some embodiments, R² is methyl, ethyl, n-propyl, or isopropyl, each of which is substituted by NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl.

In some embodiments, R² is methyl, ethyl, n-propyl, or isopropyl, each of which is substituted by —NR^{1a}R^{1b} wherein each R^{1a} and R^{1b} are independently C₁-C₃ alkyl. In some embodiments, R² is methyl, ethyl, n-propyl, or isopropyl, each of which is substituted by —N(C₁-C₃ alkyl)(C₁-C₃ alkyl). In some embodiments, R² is methyl, ethyl, n-propyl, or isopropyl, each of which is substituted by —N(methyl)(methyl), —N(methyl)(ethyl), —N(methyl)(n-propyl), —N(methyl)(isopropyl), —N(ethyl)(ethyl), —N(ethyl)(n-propyl), —N(ethyl)(isopropyl), —N(n-propyl)(n-propyl), —N(n-propyl)(isopropyl), or —N(isopropyl)(isopropyl). In some embodiments, R² is methyl substituted by —N(methyl)(methyl), —N(methyl)(ethyl), —N(methyl)(n-propyl), —N(methyl)(isopropyl), —N(ethyl)(ethyl), —N(ethyl)(n-propyl), —N(ethyl)(isopropyl), —N(n-propyl)(n-propyl), —N(n-propyl)(isopropyl), or —N(isopropyl)(isopropyl). In some embodiments, R² is methyl, ethyl, n-propyl, or isopropyl, each of which is substituted by —N(methyl)(methyl). In some embodiments, R² is methyl substituted by —N(methyl)(methyl).

In some embodiments, R² is methyl, ethyl, n-propyl, or isopropyl, each of which is substituted by —NR^{1a}R^{1b} wherein each pair of geminal R^{1a} and R^{1b} may be taken together with the nitrogen atom to which they are attached to form an N-heterocycloalkyl. In some embodiments, each pair of geminal R^{1a} and R^{1b} may be taken together to form a 3- to 7-membered N-heterocycloalkyl. In some embodiments, R² is methyl, ethyl, n-propyl, or isopropyl, each of which is substituted by 3- to 7-membered nitrogen-linked N-heterocycloalkyl. In some embodiments, R² is methyl, ethyl, n-propyl, or isopropyl, each of which is substituted by 4- to 6-membered nitrogen-linked N-heterocycloalkyl. In some embodiments, R² is methyl, ethyl, n-propyl, or isopropyl, each of which is substituted by 3- to 7-membered nitrogen-linked N-heterocycloalkyl containing 1 nitrogen atom. In some embodiments, R² is methyl, ethyl, n-propyl, or isopropyl, each of which is substituted by 3- to 7-membered nitrogen-linked N-heterocycloalkyl containing 2 nitrogen atoms. In some embodiments, R² is methyl, ethyl, n-propyl, or isopropyl, each of which is substituted by 4- to 6-membered nitrogen-linked heterocycloalkyl containing 1 nitrogen atom. In some embodiments, R² is methyl, ethyl, n-propyl, or isopropyl, each of which is substituted by 4- to 6-membered nitrogen-linked heterocycloalkyl containing 2 nitrogen atoms. In some embodiments, R² is methyl, ethyl, n-propyl, or isopropyl, each of which is substituted by nitrogen-linked azetidinyl, diazetidinyl, pyrrolidinyl, pyrazolidinyl, imidazolidinyl, piperidinyl, or piperazinyl. In some embodiments, R² is methyl, ethyl, n-propyl, or isopropyl, each of which is substituted by nitrogen-linked azetidinyl, pyrrolidinyl, or piperidinyl.

In some embodiments wherein n is 2, the two R² groups are present on the same carbon atom and are taken together with the carbon atom to which they are attached to form a C₃-C₆ spirocycloalkyl or 4- to 6-membered spiroheterocycloalkyl containing 1-2 ring heteroatoms selected from the group consisting of N, O, and S, or the two R² groups are present on vicinal carbon atoms and are taken together with the two carbon atoms to form a fused C₃-C₆ cycloalkyl. In some embodiments, wherein n is 2, two R² groups may be present on the same carbon. In some embodiments, wherein n is 2 and two R² groups are present on the same carbon, the two R² groups on the same carbon atom may be taken together with the carbon atom to which they are attached to form a C₃-C₆ spirocycloalkyl or 4- to 6-membered spiroheterocycloalkyl containing 1-2 ring heteroatoms selected from the group consisting of N, O, and S. In some embodiments, the two R² groups present on the same carbon are taken together with the carbon atom to which they are attached to form a C₃-C₆ spirocycloalkyl. In some embodiments, the two R² groups on the same carbon are taken together with the carbon atom to which they are attached to form a spirocyclopropyl (C₃-spirocycloalkyl), a spirocyclobutyl (C₄-spirocycloalkyl), a spirocyclopentyl (C₅-spirocycloalkyl), or a spirocyclohexyl (C₆-spirocycloalkyl).

In some embodiments, the two R² groups on the same carbon are taken together with the carbon atom to which they are attached to form a 4- to 6-membered spiroheterocycloalkyl containing 1-2 ring heteroatoms selected from the group consisting of N, O, and S. In some embodiments, the two R² groups on the same carbon are taken together with the carbon atom to which they are attached to form a 4-membered spiroheterocycloalkyl. In certain embodiments, the two R² groups on the same carbon are taken together with the carbon atom to which they are attached to form a spiroazetidinyl, spirooxetanyl, or spirothietanyl. In some embodiments, the two R² groups on the same carbon are taken together with the carbon atom to which they are attached to form a 5-membered spiroheterocycloalkyl. In certain embodiments, the two R² groups on the same carbon are taken together with the carbon atom to which they are attached to form a spiropyrrolidinyl, spiropyrazolidinyl, spiroimidazolidinyl, spirotetrohydrofuranyl, spirodioxolanyl, spirotetrahydrothiophenyl, or spirooxathiolanyl. In some embodiments, the two R² groups on the same carbon are taken together with the carbon atom to which they are attached to form a 6-membered spiroheterocycloalkyl. In certain embodiments, the two R² groups on the same carbon are taken together with the carbon atom to which they are attached to form a spiropiperidinyl, spiropiperazinyl, spirotetrahydropyranyl, spirodioxanyl, spirothianyl, spirodithianyl, spiromorpholinyl, or spirothiomorpholinyl.

In some embodiments, wherein n is 2, two R² groups may be present on vicinal carbons. In some embodiments wherein n is 2 and two R² groups are present on vicinal carbons, the two R² groups are taken together with the carbon atoms to which they are attached to form a fused C₃-C₆ cycloalkyl. In some embodiments, the two R² groups are taken together with the carbon atoms to which they are attached to form a fused cyclopropyl (C₃-cycloalkyl), a fused cyclobutyl (C₄-cycloalkyl), a fused cyclopentyl (C₅-cycloalkyl), or a fused cyclohexyl (C₆-cycloalkyl).

In some embodiments, R⁶ is —H, -halogen, or C₁-C₃ alkyl. In some embodiments, R⁶ is —H or halogen. In some embodiments, R⁶ is halogen. In some embodiments, R⁶ is —H or —F. In some embodiments, R⁶ is —H. In other embodiments, R⁶ is —F. In some embodiments, R⁶ is C₁-C₃ alkyl. In some embodiments, R⁶ is methyl, ethyl, n-propyl, or isopropyl. In some embodiments, R⁶ is methyl. In some embodiments, R⁷ is —H, -halogen, or C₁-C₃ alkyl. In some embodiments, R⁷ is —H or halogen. In some embodiments, R⁷ is halogen. In some embodiments, R⁷ is —H or —F.

In some embodiments, R⁷ is —H. In other embodiments, R⁷ is —F. In some embodiments, R⁷ is C₁-C₃ alkyl. In some embodiments, R⁷ is methyl, ethyl, n-propyl, or isopropyl. In some embodiments, R⁷ is methyl. In some embodiments, both R⁶ and R⁷ are H. In some embodiments, both R⁶ and R⁷ are F. In some embodiments, R⁶ is H and R⁷ is F. In some embodiments, R⁶ is F and R⁷ is H. In some embodiments, at least one of R⁶ and R⁷ is C₁-C₃ alkyl. In some embodiments, at least one of R⁶ and R⁷ is methyl. In some embodiments, at least one of R⁶ and R⁷ is —H. In other embodiments, at least one of R⁶ and R⁷ is —F.

In some embodiments, V is N or CR⁸. In some embodiments, V is N.

In some embodiments, V is CR⁸ and R⁸ is H, F, C₁-C₃-alkyl or —O(C₁-C₃-alkyl), wherein the C₁-C₃-alkyl or the C₁-C₃-alkyl of the —O(C₁-C₃-alkyl) is optionally substituted by 1-4 substituents selected from the group consisting of —F, —OH, —OR^{8a}, and —NR^{8a}R^{8b}, wherein each R^{8a} and R^{8b} are independently H or C₁-C₃ alkyl or wherein each pair of geminal R^{8a} and R^{8b} may be taken together with the nitrogen atom to which they are attached to form an N-heterocycloalkyl wherein the N-heterocycloalkyl is optionally substituted by C₁-C₃ alkyl. In some embodiments, V is CR⁸ and R⁸ is C₁-C₃-alkyl. In some embodiments, R⁸ is —CH₃, —CH₂CH₃, —CH₂CH₂CH₃, or —CH(CH₃)₂, each of which is optionally substituted by 1-4 substituents selected from the group consisting of —F, —OH, —OR^{8a}, and —NR^{8a}R^{8b}.

In some embodiments, V is CR⁸ and R⁸ is H, F, or —O(C₁-C₃-alkyl), wherein the C₁-C₃-alkyl of the —O(C₁-C₃-alkyl) is optionally substituted by 1-4 substituents selected from the group consisting of —F, —OH, —OR^{8a}, and —NR^{8a}R^{8b}, wherein each R^{8a} and R^{8b} are independently H or C₁-C₃ alkyl or wherein each pair of geminal R^{8a} and R^{8b} may be taken together with the nitrogen atom to which they are attached to form an N-heterocycloalkyl wherein the N-heterocycloalkyl is optionally substituted by C₁-C₃ alkyl. In some embodiments, R⁸ is —H. In some embodiments, R⁸ is —F. In some embodiments, R⁸ is optionally substituted —O(C₁-C₃-alkyl). In some embodiments, R⁸ is optionally substituted methoxy, ethoxy, n-propoxy, or isopropoxy. In some embodiments, R⁸ is —(O)C₁-C₃ alkyl. In some embodiments, R⁸ is —OCH₃, —OCH₂CH₃, —OCH₂CH₂CH₃, or —OCH(CH₃)₂. In certain embodiments, R⁸ is —OCH₃. In certain embodiments, R⁸ is —OCH₂CH₃. In certain embodiments, R⁸ is —OCH₂CH₂CH₃. In certain embodiments, R⁸ is —OCH(CH₃)₂.

In some embodiments, V is CR⁸ and R⁸ is —O(C₁-C₃ alkyl), wherein the —O(C₁-C₃ alkyl) is substituted by 1-4 substituents selected from the group consisting of —F, —OH, a 3- to 7-membered carbon-linked N-heterocycloalkyl, and —NR^{8a}R^{8b}, wherein each R^{8a} and R^{8b} are independently H or C₁-C₃ alkyl or wherein each pair of geminal R^{8a} and R^{8b} may be taken together with the nitrogen atom to which they are attached to form an N-heterocycloalkyl wherein the N-heterocycloalkyl is optionally substituted by C₁-C₃ alkyl.

In some embodiments, V is CR⁸ and R⁸ is —O(C₁-C₃-alkyl) substituted by 1 substituent selected from the group consisting of —F, —OH, —OR^{8a} and —NR^{8a}R^{8b}. In some embodiments, R⁸ is —O(C₁-C₃-alkyl) substituted by 2 substituents selected from the group consisting of —F, —OH, —OR^{8a} and —NR^{8a}R^{8b}. In some embodiments, R⁸ is —O(C₁-C₃-alkyl) substituted by 3 substituents selected from the group consisting of —F, —OH, —OR^{8a} and —NR^{8a}R^{8b}. In some embodiments, R⁸ is —O(C₁-C₃-alkyl) substituted by 4 substituents selected from the group consisting of —F, —OH, —OR^{8a} and —NR^{8a}R^{8b}.

In some embodiments, V is CR⁸ and R⁸ is —O(C₁-C₃-alkyl) substituted by —F. In some embodiments, R⁸ is methoxy, ethoxy, n-propoxy, or isopropoxy, each of which is substituted by 1-4 F groups. In some embodiments, R⁸ is —OCH₂F, —OCHF₂, —OCF₃, —OCH₂CH₂F, —OCH₂CHF₂, —OCH₂CF₃, —OCHFCH₃, —OCF₂CH₃, —OCHFCH₂F, —OCF₂CH₂F, —OCHFCHF₂, —OCH₂CH₂CH₂F, —OCH₂CH₂CHF₂, —OCH₂CH₂CF₃, —OCH₂CHFCH₂F, —OCH₂CHFCHF₂, —OCH₂CF₂CH₂F, —OCHFCHFCH₂F, —OCF₂CH₂CH₂F, —OCHFCH₂CH₃, —OCH₂CHFCH₃, —OCHFCHFCH₃, —OCHFCF₂CH₃, or —OCF₂CHFCH₃.

In some embodiments, V is CR⁸ and R⁸ is —O(C₁-C₃-alkyl) substituted by —OH. In some embodiments, R⁸ is methoxy, ethoxy, n-propoxy, or isopropoxy, each of which is substituted by 1-4 —OH groups. In some embodiments, R⁸ is —OCH₂OH, —OCH(OH)CH₃, —OCH₂CH₂OH, —OCH(OH)CH₂CH₃, —OCH₂CH(OH)CH₃, —OCH₂CH₂CH₂OH, —OC(CH₃)₂OH, or —OCH(CH₂OH)(CH₃). In some embodiments, R⁸ is —OCH₂CH₂OH.

In some embodiments, V is CR⁸ and R⁸ is —O(C₁-C₃-alkyl) substituted by —OR^{8a}, wherein R^{8a} is C₁-C₃ alkyl. In some embodiments, R⁸ is methoxy, ethoxy, n-propoxy, or isopropoxy, each of which is substituted by 1-4 —OR^{8a} groups. In some embodiments, R⁸ is —O(C₁-C₃-alkyl) substituted by —OR^{8a}, wherein R^{8a} is —CH₃, —CH₂CH₃, —CH₂CH₂CH₃, or —CH(CH₃)₂. In some embodiments, R⁸ is methoxy substituted by —OR^{8a}. In some embodiments, R⁸ is ethoxy substituted by —OR^{8a}. In some embodiments, R⁸ is n-propoxy substituted by —OR^{8a}. In some embodiments, R⁸ is isopropoxy substituted by —OR^{8a}. In certain embodiments, R⁸ is —O(C₁-C₃-alkyl) substituted by —OR^{8a}, wherein R^{8a} is —CH₃. In certain embodiments, R⁸ is —O(C₁-C₃-alkyl) substituted by —OR^{8a}, wherein R^{8a} is —CH₂CH₃. In certain embodiments, R⁸ is —O(C₁-C₃-alkyl) substituted by —OR^{8a}, wherein R^{8a} is —CH₂CH₂CH₃. In certain embodiments, R⁸ is —O(C₁-C₃-alkyl) substituted by —OR^{8a}, wherein R^{8a} is —CH(CH₃)₂. In some embodiments, R⁸ is —OCH₂OCH₃, —OCH₂OCH₂CH₃, —OCH₂OCH₂CH₂CH₃, —OCH₂OCH(CH₃)₂, —OCH(OCH₃)CH₃, —OCH(OCH₂CH₃)CH₃, —OCH(OCH₂CH₂CH₃)CH₃, —OCH(CH₃)OCH(CH₃)₂, —OCH₂CH₂OCH₃, —OCH₂CH₂OCH₂CH₃, —OCH₂CH₂OCH₂CH₂CH₃, —OCH₂CH₂OCH(CH₃)₂, —OCH(OCH₃)CH₂CH₃, —OCH(CH₂CH₃)OCH₂CH₃, —OCH(OCH₂CH₂CH₃)CH₂CH₃, —OCH(OCH(CH₃)₂)CH₂CH₃, —OCH₂CH(CH₃)OCH₃, —OCH₂CH(CH₃)OCH₂CH₃, —OCH₂CH(CH₃)OCH₂CH₂CH₃, —OCH₂CH(CH₃)OCH(CH₃)₂, —OCH₂CH₂CH₂OCH₃, —OCH₂CH₂CH₂OCH₂CH₃, —OCH₂CH₂CH₂OCH₂CH₂CH₃, —OCH₂CH₂CH₂OCH(CH₃)₂, —OC(OCH₃)(CH₃)₂, —OC(OCH₂CH₃)(CH₃)₂, —OC(OCH₂CH₂CH₃)(CH₃)₂, —OC(OCH(CH₃)₂)(CH₃)₂, —OCH(CH₃)CH₂OCH₃, —OCH(CH₃)CH₂OCH₂CH₃, —OCH(CH₃)CH₂OCH₂CH₂CH₃, or —OCH(CH₃)CH₂OCH(CH₃)₂.

In some embodiments, V is CR⁸ and R⁸ is —O(C₁-C₃-alkyl) substituted by 1-4 —NR^{8a}R^{8b} groups. In some embodiments, R⁸ is —O(C₁-C₃-alkyl) substituted by NR^{8a}R^{8b}, wherein each R^{8a} and R^{8b} are independently H or C₁-C₃ alkyl. In some embodiments, R⁸ is methoxy, ethoxy, n-propoxy, or isopropoxy, each of which is substituted by —NR^{8a}R^{8b}, wherein each R^{8a} and R^{8b} are independently H or C₁-C₃ alkyl. In some embodiments, R⁸ is methoxy, ethoxy, n-propoxy, or isopropoxy, each of which is substituted by —NH₂, —NH(C₁-C₃ alkyl), or —N(C₁-C₃ alkyl)(C₁-C₃ alkyl). In some embodiments, R⁸ is methoxy, ethoxy, n-propoxy, or isopropoxy, each of which is substituted by —NH₂, —NH(methyl), —NH(ethyl), —NH(n-propyl), —NH(isopropyl), —N(methyl)(methyl), —N(methyl)(ethyl), —N(methyl)(n-propyl), —N(methyl)(isopropyl), —N(ethyl)(ethyl), —N(ethyl)(n-propyl), —N(ethyl)(isopropyl), —N(n-propyl)(n-propyl), —N(n-propyl)(isopropyl), or —N(isopropyl)(isopropyl). In some embodiments, R⁸ is methoxy substituted by —NH₂, —NH(methyl), —NH(ethyl), —NH(n-propyl), —NH(isopropyl), —N(methyl)(methyl), —N(methyl)(ethyl), —N(methyl)(n-propyl), —N(methyl)(isopropyl), —N(ethyl)(ethyl), —N(ethyl)(n-propyl), —N(ethyl)(isopropyl), —N(n-propyl)(n-propyl), —N(n-propyl)(isopropyl), or —N(isopropyl)(isopropyl). In some embodiments, R⁸ is methoxy, ethoxy, n-propoxy, or isopropoxy, each of which is substituted by —N(methyl)(methyl). In certain embodiments, R⁸ is —O(C₁-C₃-alkyl) substituted by —NR^{8a}R^{8b}, wherein each R^{8a} and R^{8b} are —CH₃. In some embodiments, R⁸ is methoxy substituted by —N(methyl)(methyl). In some embodiments, R⁸ is ethoxy or n-propoxy substituted by —N(C₁-C₃ alkyl)(C₁-C₃ alkyl). In some embodiments, R⁸ is ethoxy or n-propoxy substituted by —N(methyl)(methyl). In some embodiments, R⁸ is —OCH₂CH₂N(CH₃)₂, or —OCH₂CH₂CH₂N(CH₃)₂. In some embodiments, R⁸ is ethoxy substituted by —NH(C₁-C₃ alkyl). In some embodiments, R⁸ is ethoxy substituted by —NH(methyl). In some embodiments, R⁸ is —OCH₂CH₂NH(CH₃).

In other embodiments, V is CR⁸ and R⁸ is —O(C₁-C₃-alkyl) substituted by —NR^{8a}R^{8b}, wherein each pair of geminal R^{8a} and R^{8b} may be taken together with the nitrogen atom to which they are attached to form an N-heterocycloalkyl wherein the N-heterocycloalkyl is optionally substituted by C₁-C₃ alkyl. In some embodiments, R⁸ is methoxy, ethoxy, n-propoxy, or isopropoxy, each of which is substituted by —NR^{8a}R^{8b}, wherein each pair of geminal R^{8a} and R^{8b} may be taken together with the nitrogen atom to which they are attached to form an N-heterocycloalkyl wherein the N-heterocycloalkyl is optionally substituted by C₁-C₃ alkyl. In some embodiments, each pair of geminal R^{8a} and R^{8b} may be taken together to form a 3- to 7-membered N-heterocycloalkyl wherein the 3- to 7-membered N-heterocycloalkyl is optionally substituted by C₁-C₃ alkyl. In some embodiments, R⁸ is methoxy, ethoxy, n-propoxy, or isopropoxy, each of which is substituted by 3- to 7-membered nitrogen-linked N-heterocycloalkyl wherein the 3- to 7-membered nitrogen-linked N-heterocycloalkyl is optionally substituted by C₁-C₃ alkyl. In some embodiments, R⁸ is methoxy, ethoxy, n-propoxy, or isopropoxy, each of which is substituted by 4- to 6-membered nitrogen-linked N-heterocycloalkyl wherein the 4- to 6-membered nitrogen-linked N-heterocycloalkyl is optionally substituted by C₁-C₃ alkyl. In some embodiments, R⁸ is methoxy, ethoxy, n-propoxy, or isopropoxy, each of which is substituted by 3- to 7-membered nitrogen-linked N-heterocycloalkyl containing 1 nitrogen atom wherein the 3- to 7-membered nitrogen-linked N-heterocycloalkyl is optionally substituted by C₁-C₃ alkyl. In some embodiments, R⁸ is methoxy, ethoxy, n-propoxy, or isopropoxy, each of which is substituted by 3- to 7-membered nitrogen-linked N-heterocycloalkyl containing 2 nitrogen atoms wherein the 3- to 7-membered nitrogen-linked N-heterocycloalkyl is optionally substituted by C₁-C₃ alkyl. In some embodiments, R⁸ is methoxy, ethoxy, n-propoxy, or isopropoxy, each of which is substituted by 4- to 6-membered nitrogen-linked heterocycloalkyl containing 1 nitrogen atom wherein the 4- to 6-membered nitrogen-linked heterocycloalkyl is optionally substituted by C₁-C₃ alkyl. In some embodiments, R⁸ is methoxy, ethoxy, n-propoxy, or isopropoxy, each of which is substituted by 4- to 6-membered nitrogen-linked heterocycloalkyl containing 2 nitrogen atoms wherein the 4- to 6-membered nitrogen-linked heterocycloalkyl is optionally substituted by C₁-C₃ alkyl. In some embodiments, R⁸ is methoxy, ethoxy, n-propoxy, or isopropoxy, each of which is substituted by nitrogen-linked azetidinyl, diazetidinyl, pyrrolidinyl, pyrazolidinyl, imidazolidinyl, piperidinyl, or piperazinyl. In some embodiments, R⁸ is methoxy, ethoxy, n-propoxy, or isopropoxy, each of which is substituted by nitrogen-linked azetidinyl, pyrrolidinyl, or piperidinyl. In some embodiments, R⁸ is methoxy, ethoxy, n-propoxy, or isopropoxy, each of which is substituted by nitrogen-linked azetidinyl, diazetidinyl, pyrrolidinyl, pyrazolidinyl, imidazolidinyl, piperidinyl, or piperazinyl wherein the nitrogen-linked azetidinyl, diazetidinyl, pyrrolidinyl, pyrazolidinyl, imidazolidinyl, piperidinyl, and piperazinyl are substituted by C₁-C₃ alkyl. In some embodiments, R⁸ is methoxy, ethoxy, n-propoxy, or isopropoxy, each of which is substituted by nitrogen-linked azetidinyl, pyrrolidinyl, or piperidinyl wherein the nitrogen-linked azetidinyl, pyrrolidinyl, and piperidinyl are substituted by C₁-C₃ alkyl. In some embodiments, R⁸ is methoxy, ethoxy, n-propoxy, or isopropoxy, each of which is substituted by nitrogen-linked azetidinyl, pyrrolidinyl, or piperidinyl wherein the nitrogen-linked azetidinyl, pyrrolidinyl, and piperidinyl are substituted by CH₃.

In some embodiments, V is CR⁸ and R⁸ is methoxy, ethoxy, n-propoxy, or isopropoxy, each of which is substituted by 4- to 6-membered nitrogen-linked N-heterocycloalkyl containing 1-2 additional ring heteroatoms selected from the group consisting of N and O wherein the 4- to 6-membered nitrogen-linked N-heterocycloalkyl is optionally substituted by C₁-C₃ alkyl. In some embodiments, R⁸ is methoxy, ethoxy, n-propoxy, or isopropoxy, each of which is substituted by 4- to 6-membered nitrogen-linked N-heterocycloalkyl containing 1 oxygen atom wherein the 4- to 6-membered nitrogen-linked N-heterocycloalkyl is optionally substituted by C₁-C₃ alkyl. In some embodiments, R⁸ is methyl, ethyl, n-propyl, or isopropyl, each of which is substituted by 4- to 6-membered nitrogen-linked N-heterocycloalkyl containing 2 nitrogen atoms wherein the 4- to 6-membered nitrogen-linked N-heterocycloalkyl is optionally substituted by C₁-C₃ alkyl. In some embodiments, R⁸ is methoxy, ethoxy, n-propoxy, or isopropoxy, each of which is substituted by diazetidinyl, pyrazolidinyl, imidazolidinyl, piperazinyl, oxetanyl, or morpholinyl. In some embodiments, R⁸ is methoxy, ethoxy, n-propoxy, or isopropoxy, each of which is substituted by diazetidinyl, pyrazolidinyl, imidazolidinyl, piperazinyl, oxetanyl, or morpholinyl wherein the diazetidinyl, pyrazolidinyl, imidazolidinyl, piperazinyl, oxetanyl, or morpholinyl are substituted by C₁-C₃ alkyl. In some embodiments, R⁸ is methoxy, ethoxy, n-propoxy, or isopropoxy, each of which is substituted by diazetidinyl, pyrazolidinyl, imidazolidinyl, piperazinyl, oxetanyl, or morpholinyl wherein the diazetidinyl, pyrazolidinyl, imidazolidinyl, piperazinyl, oxetanyl, or morpholinyl are substituted by CH₃.

In other embodiments, V is CR⁸ and R⁸ is H. In other embodiments, R⁸ is —O(C₂-C₃ alkyl), wherein the C₂-C₃ alkyl is substituted by 1-2 substituents selected from the group consisting of —OH and —NR^{8a}R^{8b}, wherein each R^{8a} and R^{8b} are independently H, or C₁-C₃ alkyl. In other embodiments, R⁸ is —OCH₂CH₂OH, —OCH₂CH₂N(CH₃)₂, or —OCH₂CH₂CH₂N(CH₃)₂.

In some embodiments, R⁸ is —H or —F. In certain embodiments wherein V is C—R⁸, R⁸ is —H. In other embodiments wherein V is C—R⁸, R⁸ is —F.

In some embodiments, R¹⁰ is —H or halogen. In some embodiments, R¹⁰ is —H. In other embodiments, R¹⁰ is halogen. In some embodiments, R¹⁰ is —F. In some embodiments, R¹⁰ is —Cl. In some embodiments, R¹⁰ is —H, —F, or —Cl. In some embodiments, R¹⁰ is —H or —F.

In some embodiments, the compound of formula (I), formula (II-a), formula (III), or formula (IV) is a compound of formula (I-a):

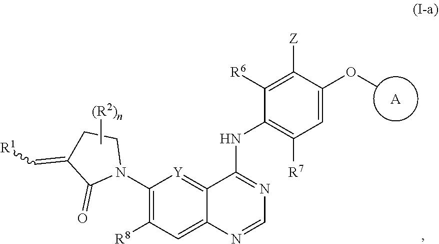

wherein R¹, R², n, R⁶, R⁷, R⁸, Y, Z, and ring A are as defined for formula (I), formula (II-a), formula (III), or formula (IV).

In some embodiments, the compound of formula (I), formula (II-a), formula (III), or formula (IV) is a compound of formula (I-b):

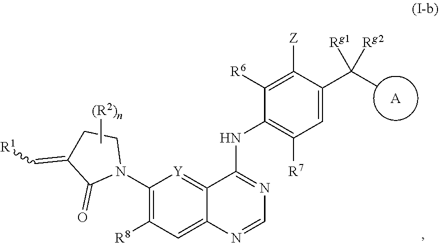

wherein R¹, R², n, R⁶, R⁷, R⁸, R^{g1}, R^{g2}, Y, Z, and ring A are as defined for formula (I), formula (II-a), formula (III), or formula (IV).

In some embodiments, the compound of formula (I), formula (II-a), formula (III), or formula (IV) is a compound of formula (I-c):

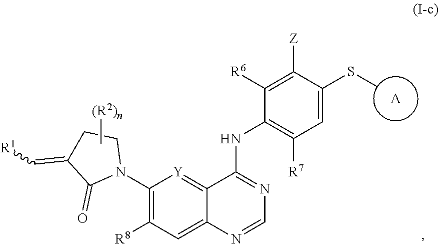

wherein R¹, R², n, R⁶, R⁷, R⁸, Y, Z, and ring A are as defined for formula (I), formula (II-a), formula (III), or formula (IV).

In some embodiments, the compound of formula (I), formula (II-a), formula (III), or formula (IV) is a compound of formula (I-d):

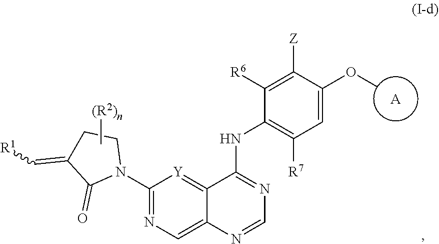

wherein R¹, R², n, R⁶, R⁷, Y, Z, and ring A are as defined for formula (I), formula (II-a), formula (III), or formula (IV).

In some embodiments, the compound of formula (I), formula (II-a), formula (III), or formula (IV) is a compound of formula (I-e):

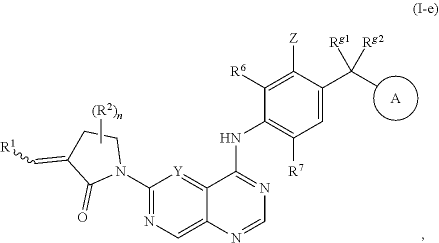

wherein R¹, R², n, R⁶, R⁷, R^{g1}, R^{g2}, Y, Z, and ring A are as defined for formula (I), formula (II-a), formula (III), or formula (IV).

In some embodiments, the compound of formula (I), formula (II-a), formula (III), or formula (IV) is a compound of formula (I-f):

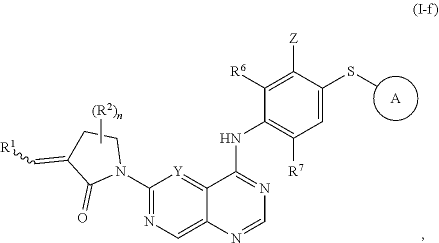

wherein R¹, R², n, R⁶, R⁷, Y, Z, and ring A are as defined for formula (I), formula (II-a), formula (III), or formula (IV).

In some embodiments, the compound of formula (I), formula (II-a), formula (III), or formula (IV) is a compound of formula (I-g):

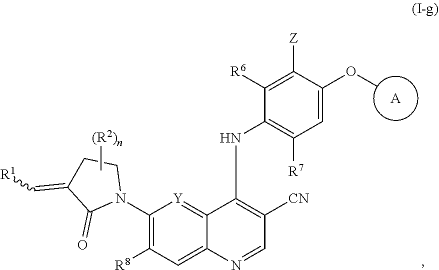

wherein R¹, R², n, R⁶, R⁷, R⁸, Y, Z, and ring A are as defined for formula (I), formula (II-a), formula (III), or formula (IV).

In some embodiments, the compound of formula (I), formula (II-a), formula (III), or formula (IV) is a compound of formula (I-h):

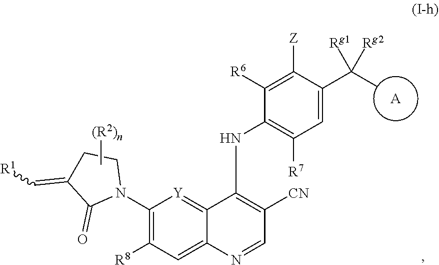

wherein R¹, R², n, R⁶, R⁷, R⁸, R^{g1}, R^{g2}, Y, Z, and ring A are as defined for formula (I), formula (II-a), formula (III), or formula (IV).

In some embodiments, the compound of formula (I), formula (II-a), formula (III), or formula (IV) is a compound of formula (I-i):

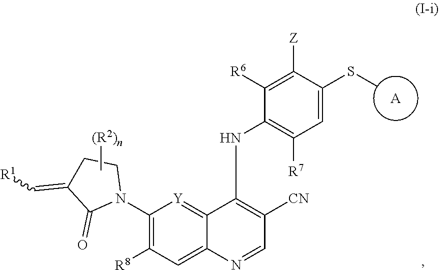

wherein R¹, R², n, R⁶, R⁷, R⁸, Y, Z, and ring A are as defined for formula (I), formula (II-a), formula (III), or formula (IV).

In some embodiments, the compound of formula (I-a) is a compound of formula (I-a-1) or (I-a-2):

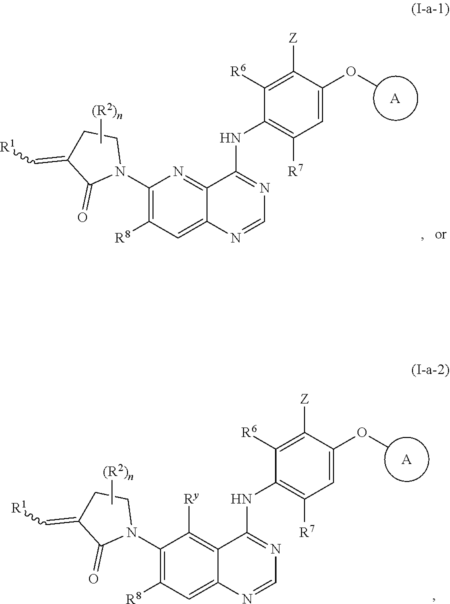

wherein R¹, R², n, R⁶, R⁷, R⁸, R^{y}, Z, and ring A are as defined for formula (I), formula (II-a), formula (III), or formula (IV). In some embodiments, the compound is a compound of formula (I-a-1). In some embodiments, the compound is a compound of formula (I-a-2). In any variation of formula (I-a-1) or (I-a-2), Z is —H, —F, —Cl, or —CH₃, and Ring A is

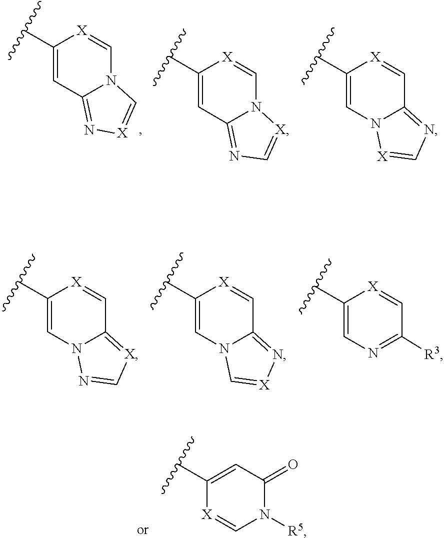

wherein each X is independently N or CH. In some embodiments, Ring A is

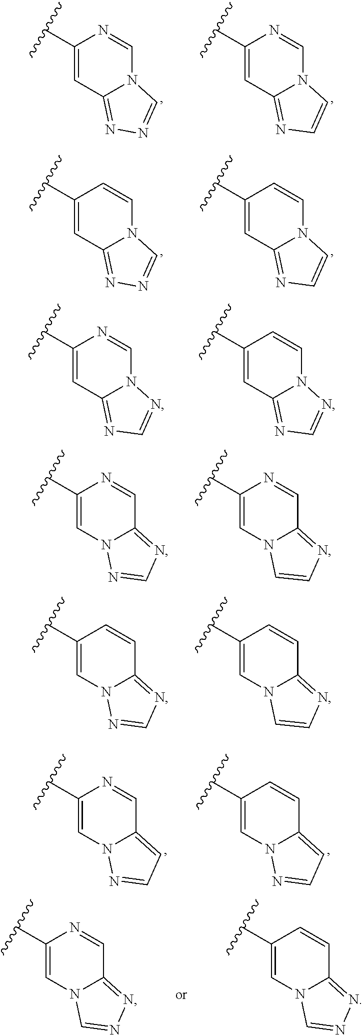

In some embodiments, Ring A is

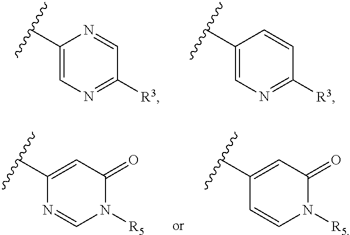

In some embodiments, Ring A is

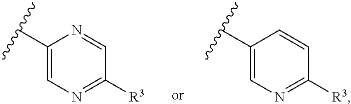

and R³ is —CH₃, —CH₂CH₃, —CHF₂, or —CD₃. In some embodiments, Ring A is

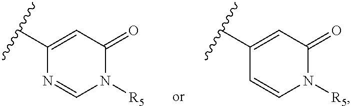

and R⁵ is —CH₃, —CH₂CH₃, —CHF₂, or —CD₃.

In some embodiments, the compound of formula (I-b) is a compound of formula (I-b-1) or (I-b-2):

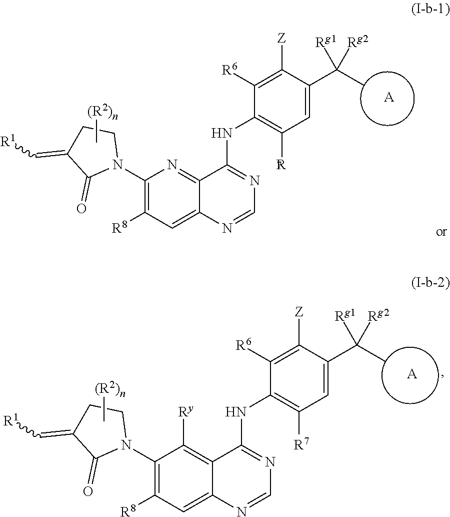

wherein R¹, R², n, R⁶, R⁷, R⁸, R^{g1}, R^{g2}, R^{y}, Z, and ring A are as defined for formula (I), formula (II-a), formula (III), or formula (IV). In some embodiments, the compound is a compound of formula (I-b-1). In some embodiments, the compound is a compound of formula (I-b-2). In any variation of formula (I-b-1) or (I-b-2), Z is —H, —F, —Cl, or —CH₃, and Ring A is

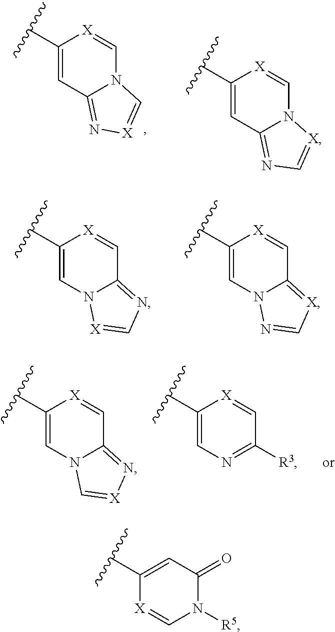

wherein each X is independently N or CH. In some embodiments, Ring A is

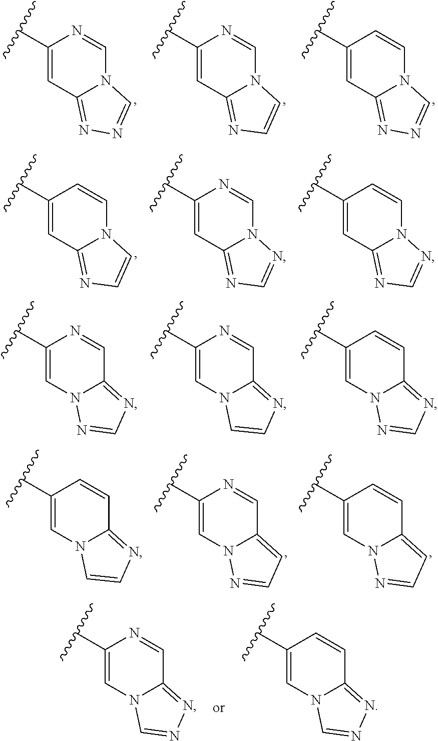

In some embodiments, Ring A is

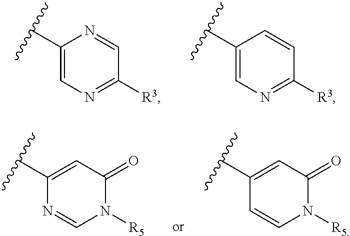

In some embodiments, Ring A is

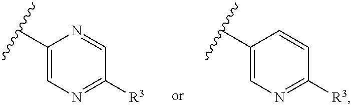

and R³ is —CH₃, —CH₂CH₃, —CHF₂, or —CD₃. In some embodiments, Ring A is

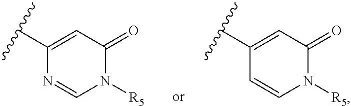

and R⁵ is —CH₃, —CH₂CH₃, —CHF₂, or —CD₃.

In some embodiments, the compound of formula (I-c) is a compound of formula (I-c-1) or (I-c-2):

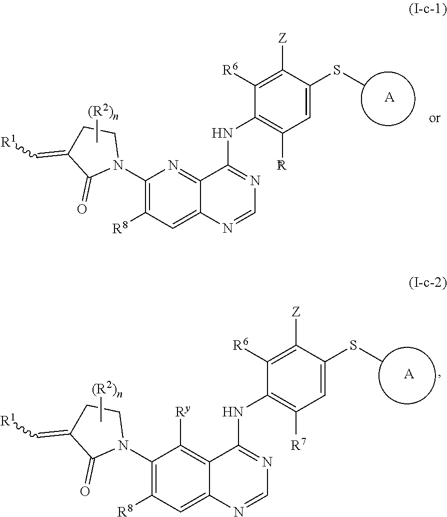

wherein R¹, R², n, R⁶, R⁷, R⁸, R^{y}, Z, and ring A are as defined for formula (I), formula (II-a), formula (III), or formula (IV). In some embodiments, the compound is a compound of formula (I-c-1). In some embodiments, the compound is a compound of formula (I-c-2). In any variation of formula (I-c-1) or (I-c-2), Z is —H, —F, —Cl, or —CH₃, and Ring A is

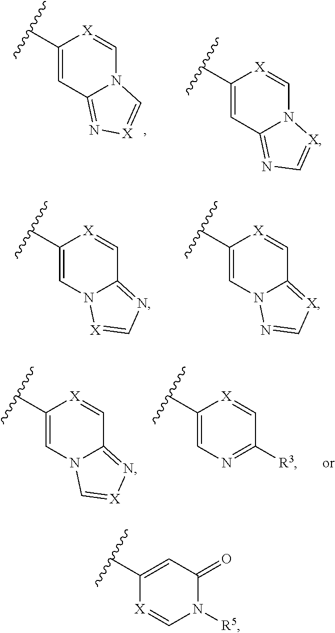

wherein each X is independently N or CH. In some embodiments, Ring A is

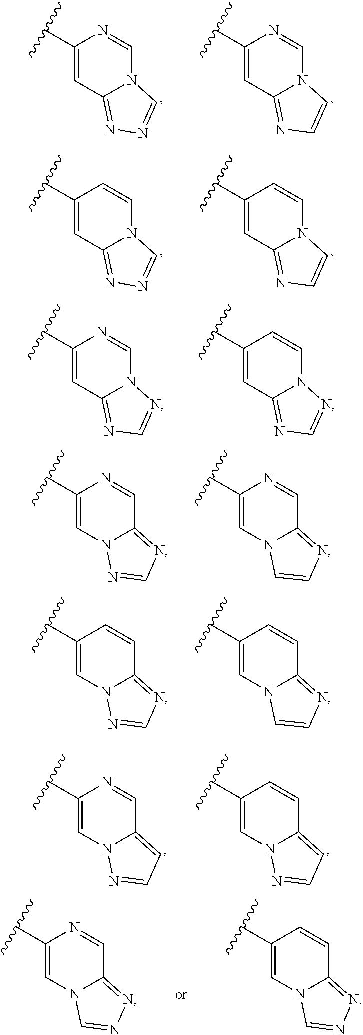

In some embodiments, Ring A is

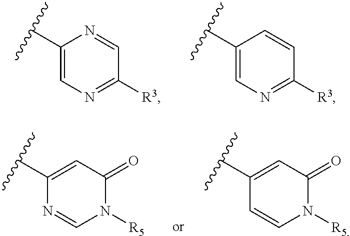

In some embodiments, Ring A is

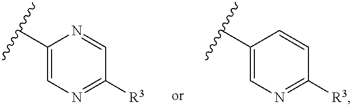

and R³ is —CH₃, —CH₂CH₃, —CHF₂, or —CD₃. In some embodiments, Ring A is

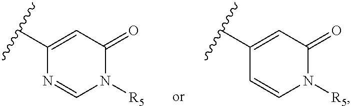

and R⁵ is —CH₃, —CH₂CH₃, —CHF₂, or —CD₃.

In some embodiments, the compound of formula (I-d) is a compound of formula (I-d-1) or (I-d-2):

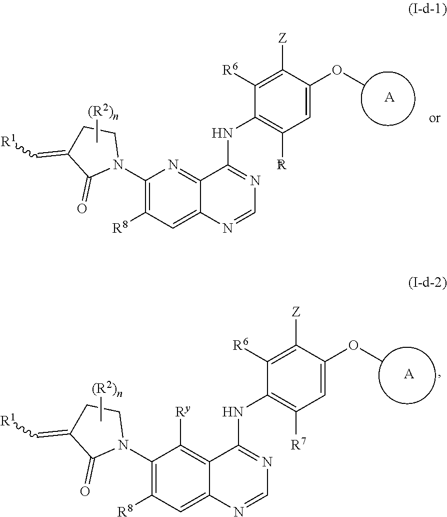

wherein R¹, R², n, R⁶, R⁷, R^{y}, Z, and ring A are as defined for formula (I), formula (II-a), formula (III), or formula (IV). In some embodiments, the compound is a compound of formula (I-d-1). In some embodiments, the compound is a compound of formula (I-d-2). In any variation of formula (I-d-1) or (I-d-2), Z is —H, —F, —Cl, or —CH₃, and Ring A is

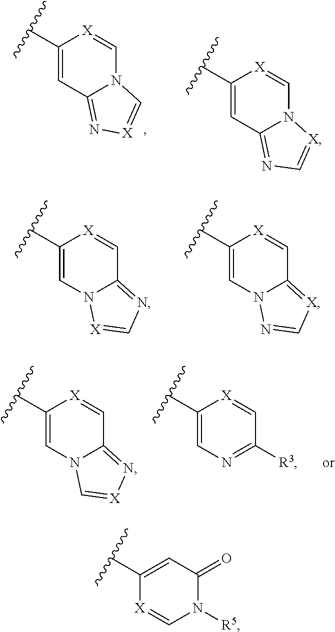

wherein each X is independently N or CH. In some embodiments, Ring A is

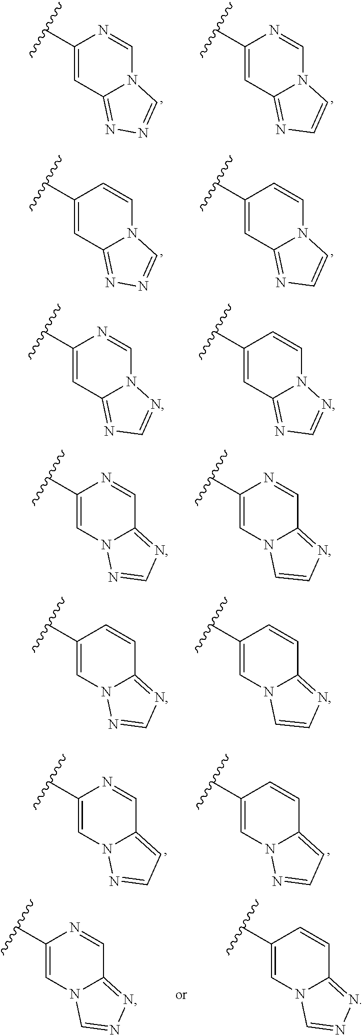

In some embodiments, Ring A is

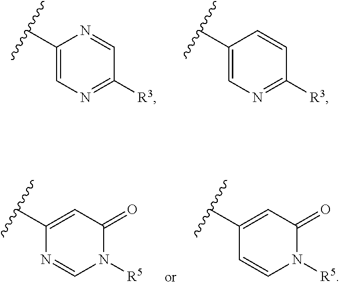

In some embodiments, Ring A is

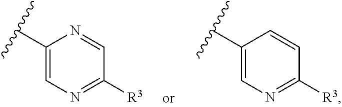

and R³ is —CH₃, —CH₂CH₃, —CHF₂, or —CD₃. In some embodiments, Ring A is

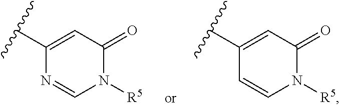

and R⁵ is —CH₃, —CH₂CH₃, —CHF₂, or —CD₃.

In some embodiments, the compound of formula (I-e) is a compound of formula (I-e-1) or (I-e-2):

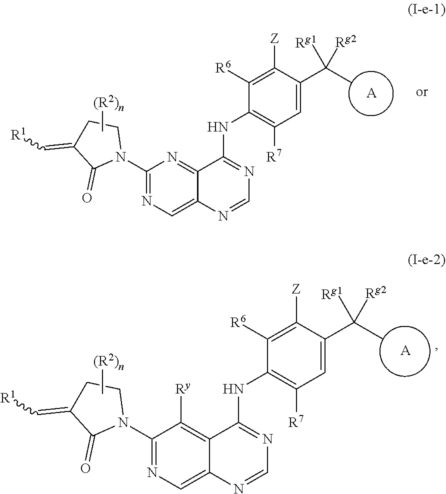

wherein R¹, R², n, R⁶, R⁷, R^{g1}, R^{g2}, R^{y}, Z, and ring A are as defined for formula (I), formula (II-a), formula (III), or formula (IV). In some embodiments, the compound is a compound of formula (I-e-1). In some embodiments, the compound is a compound of formula (I-e-2). In any variation of formula (I-e-1) or (I-e-2), Z is —H, —F, —Cl, or —CH₃, and Ring A is

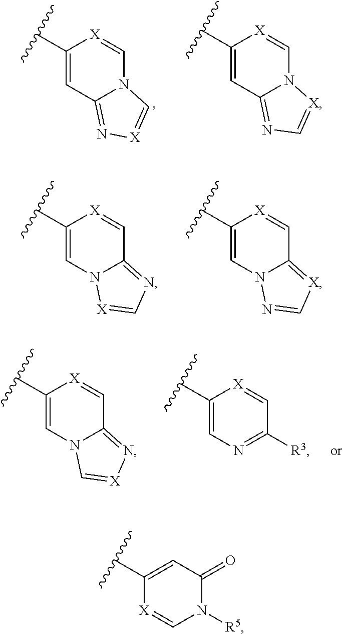

wherein each X is independently N or CH. In some embodiments, Ring A is

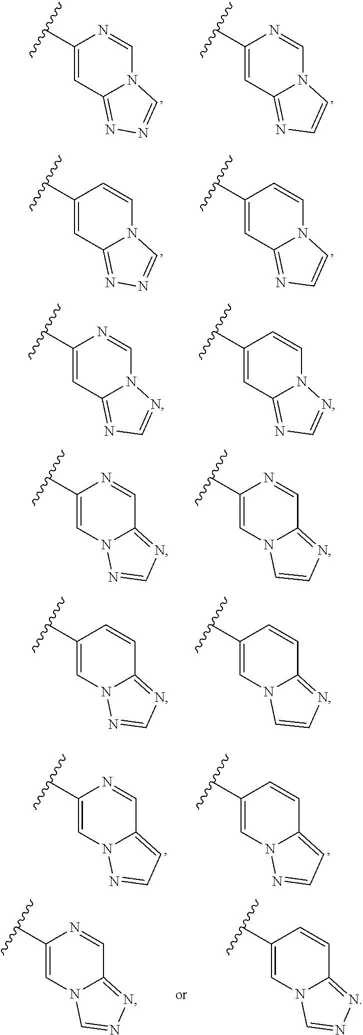

In some embodiments, Ring A is

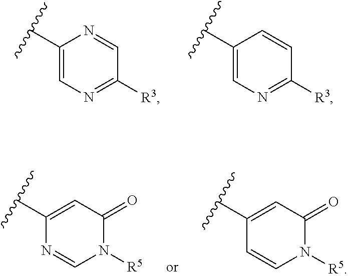

In some embodiments, Ring A is

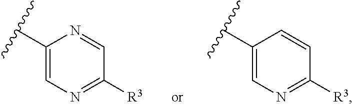

and R³ is —CH₃, —CH₂CH₃, —CHF₂, or —CD₃. In some embodiments, Ring A is

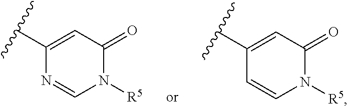

and R⁵ is —CH₃, —CH₂CH₃, —CHF₂, or —CD₃.

In some embodiments, the compound of formula (I-f) is a compound of formula (I-f-1) or (I-f-2):

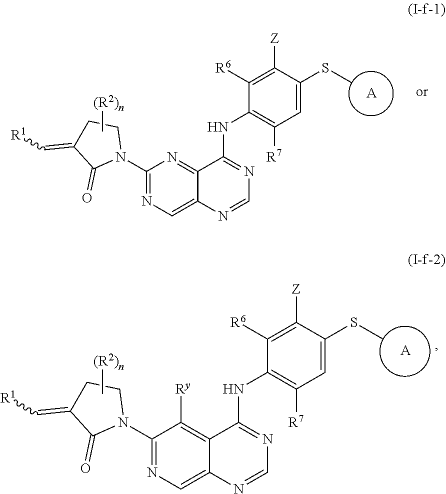

wherein R¹, R², n, R⁶, R⁷, R^{y}, Z, and ring A are as defined for formula (I), formula (II-a), formula (III), or formula (IV). In some embodiments, the compound is a compound of formula (I-f-1). In some embodiments, the compound is a compound of formula (I-f-2). In any variation of formula (I-f-1) or (I-f-2), Z is —H, —F, —Cl, or —CH₃, and Ring A is

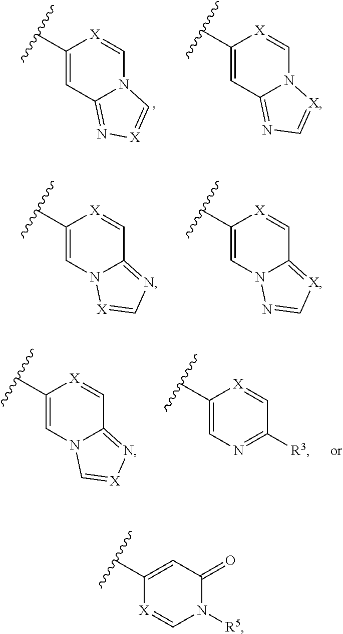

wherein each X is independently N or CH. In some embodiments, Ring A is

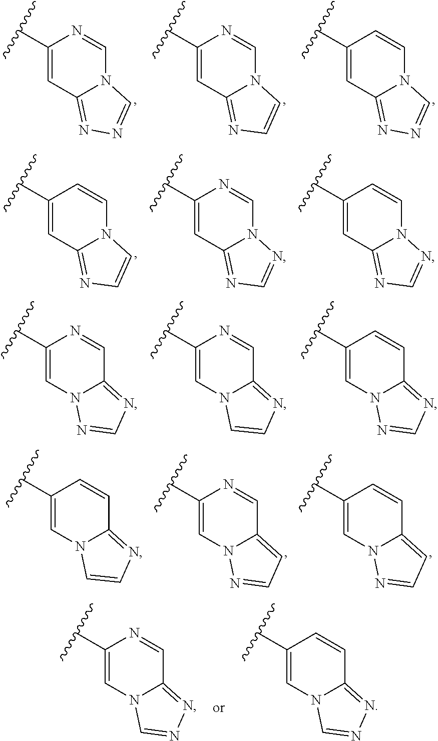

In some embodiments, Ring A is

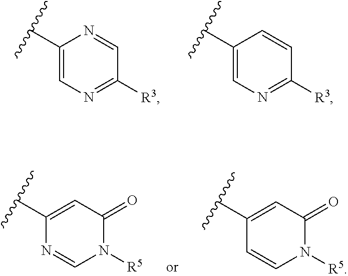

In some embodiments, Ring A is

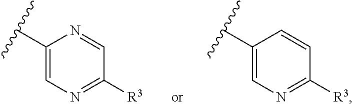

and R³ is —CH₃, —CH₂CH₃, —CHF₂, or —CD₃. In some embodiments, Ring A is

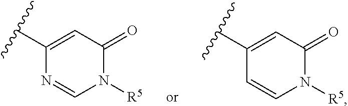

and R⁵ is —CH₃, —CH₂CH₃, —CHF₂, or —CD₃.

In some embodiments, the compound of formula (I-g) is a compound of formula (I-g-1) or (I-g-2):

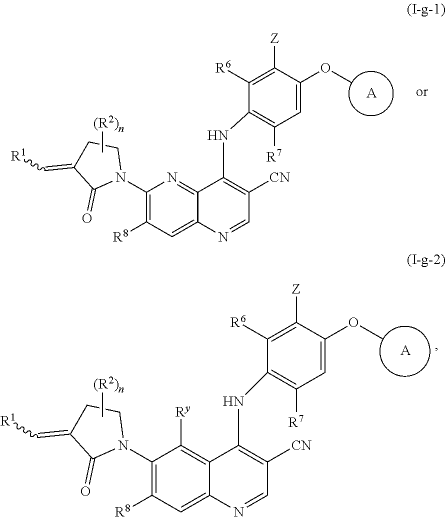

wherein R¹, R², n, R⁶, R⁷, R⁸, R^{y}, Z, and ring A are as defined for formula (I), formula (II-a), formula (III), or formula (IV). In some embodiments, the compound is a compound of formula (I-g-1). In some embodiments, the compound is a compound of formula (I-g-2). In any variation of formula (I-g-1) or (I-g-2), Z is —H, —F, —Cl, or —CH₃, and Ring A is

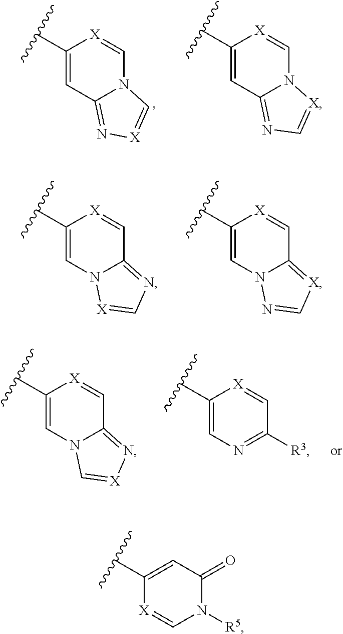

wherein each X is independently N or CH. In some embodiments, Ring A is

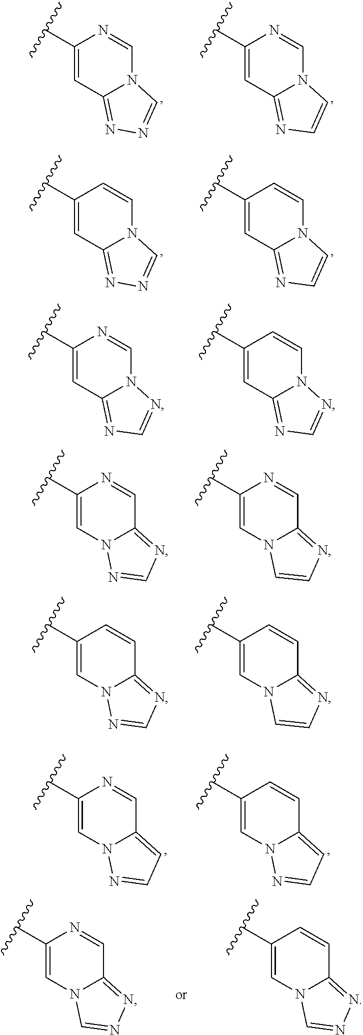

In some embodiments, Ring A is

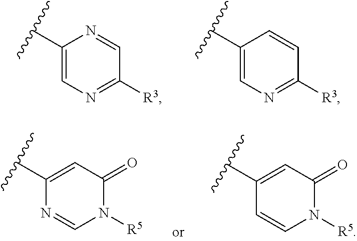

In some embodiments, Ring A is

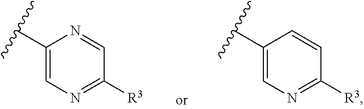

and R³ is —CH₃, —CH₂CH₃, —CHF₂, or —CD₃. In some embodiments, Ring A is

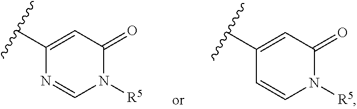

and R⁵ is —CH₃, —CH₂CH₃, —CHF₂, or —CD₃.

In some embodiments, the compound of formula (I-h) is a compound of formula (I-h-1) or (I-h-2):

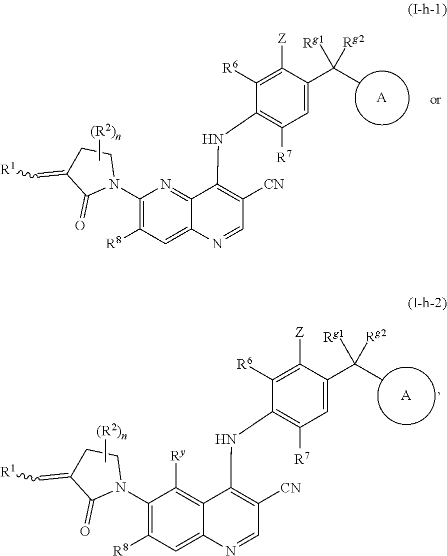

wherein R¹, R², n, R⁶, R⁷, R⁸, R^{g1}, R^{g2}, R^{y}, Z, and ring A are as defined for formula (I), formula (II-a), formula (III), or formula (IV). In some embodiments, the compound is a compound of formula (I-h-1). In some embodiments, the compound is a compound of formula (I-h-2). In any variation of formula (I-h-1) or (I-h-2), Z is —H, —F, —Cl, or —CH₃, and Ring A is

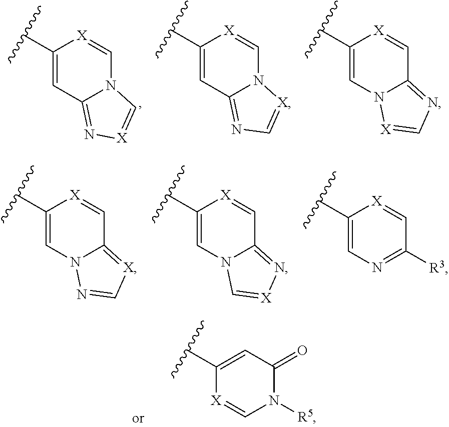

wherein each X is independently N or CH. In some embodiments, Ring A is

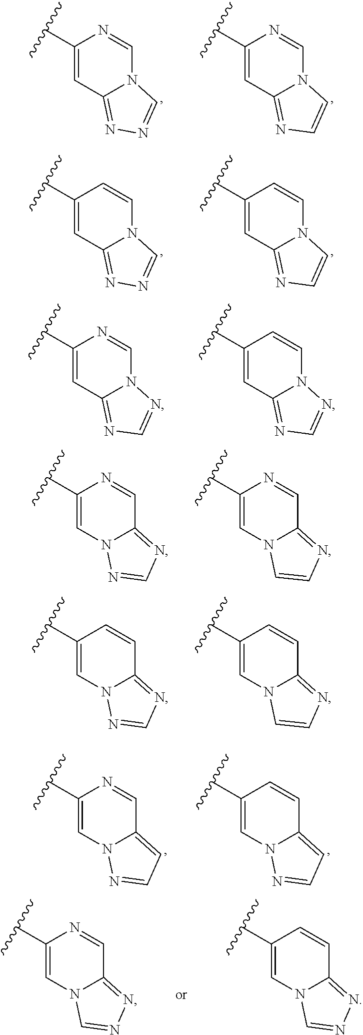

In some embodiments, Ring A is

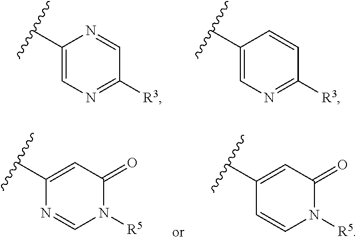

In some embodiments, Ring A is

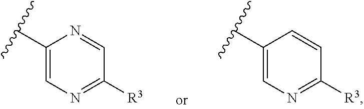

and R³ is —CH₃, —CH₂CH₃, —CHF₂, or —CD₃. In some embodiments, Ring A is

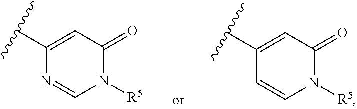

and R⁵ is —CH₃, —CH₂CH₃, —CHF₂, or —CD₃.

In some embodiments, the compound of formula (I-i) is a compound of formula (I-i 1) or (I-i-2):

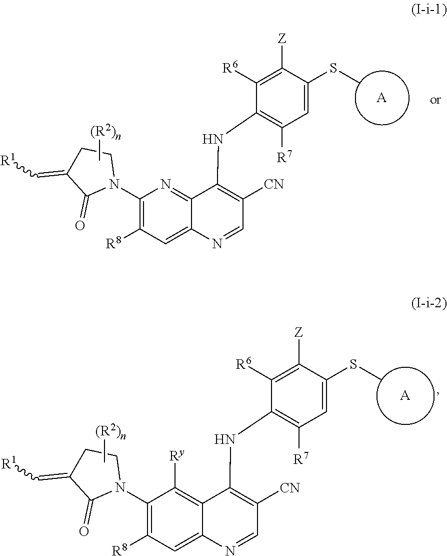

wherein R¹, R², n, R⁶, R⁷, R⁸, R^{y}, Z, and ring A are as defined for formula (I), formula (II-a), formula (III), or formula (IV). In some embodiments, the compound is a compound of formula (I-i-1). In some embodiments, the compound is a compound of formula (I-i-2). In any variation of formula (I-i-1) or (I-i-2), Z is —H, —F, —Cl, or —CH₃, and Ring A is

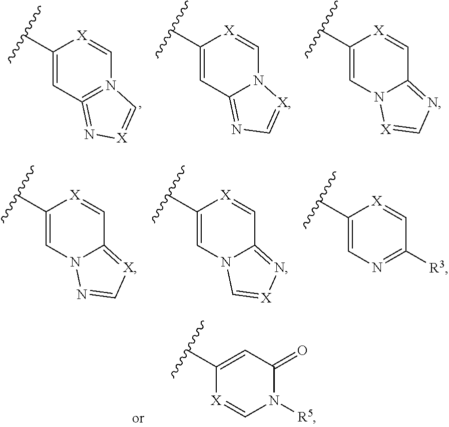

wherein each X is independently N or CH. In some embodiments, Ring A is

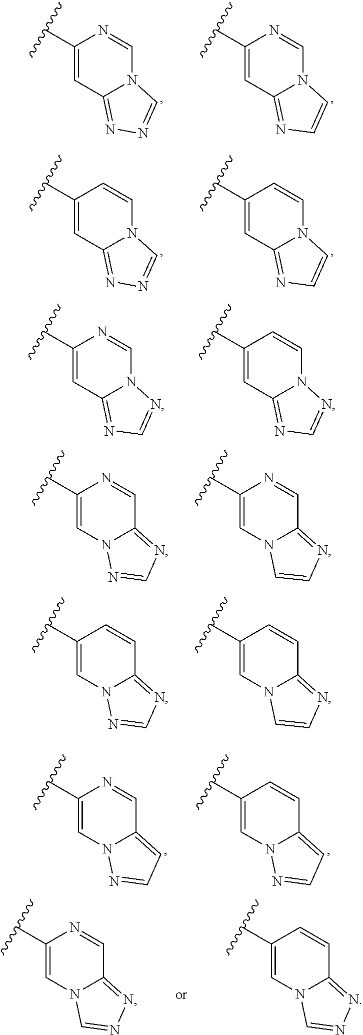

In some embodiments, Ring A is

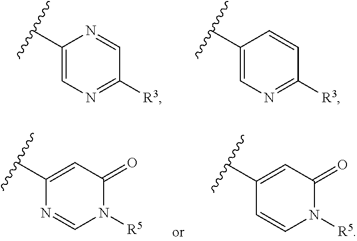

In some embodiments, Ring A is

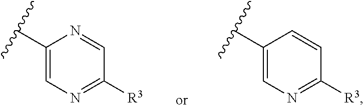

and R³ is —CH₃, —CH₂CH₃, —CHF₂, or —CD₃. In some embodiments, Ring A is

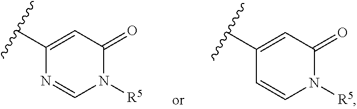

and R⁵ is —CH₃, —CH₂CH₃, —CHF₂, or —CD₃.

In some embodiments, the compound of formula (I-a) is a compound of formula (I-j-1), (I-j-2), (I-j-3), (I-j-4), (I-j-5), (I-j-6), (I-j-7), (I-j-8), (I-j-9), (I j-10), (I j-11), (I-j-12), (I-j-13), (I-j-14), (I-j-15), (I-j-16), (I-j-17), or (I-j-18):

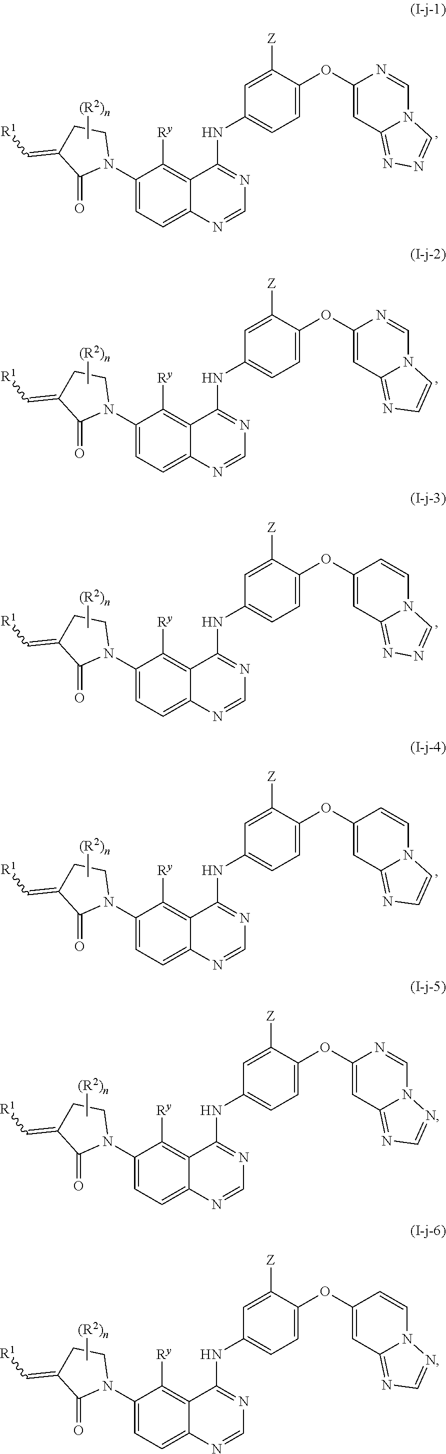

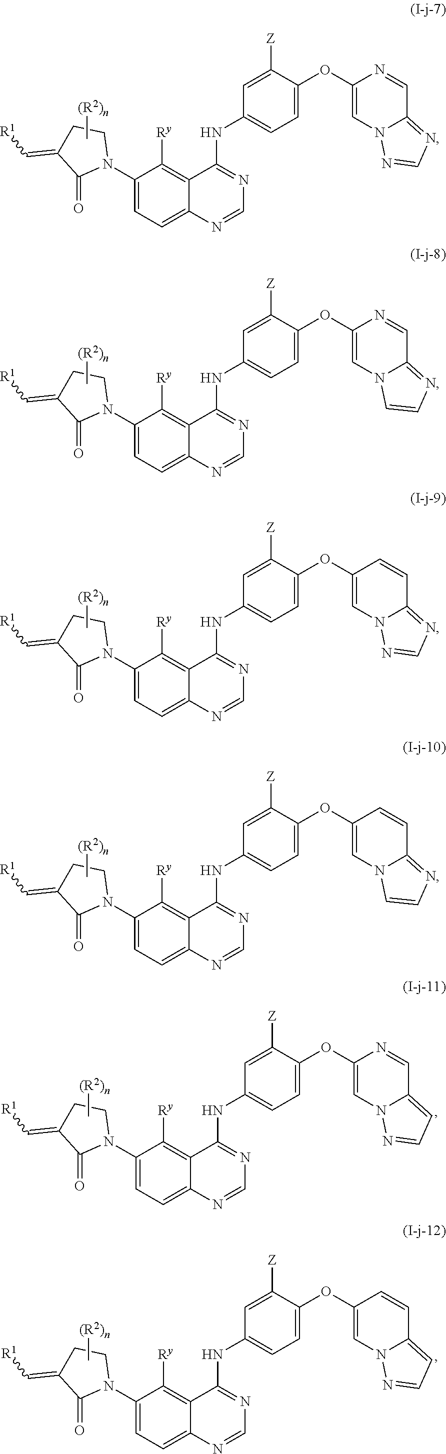

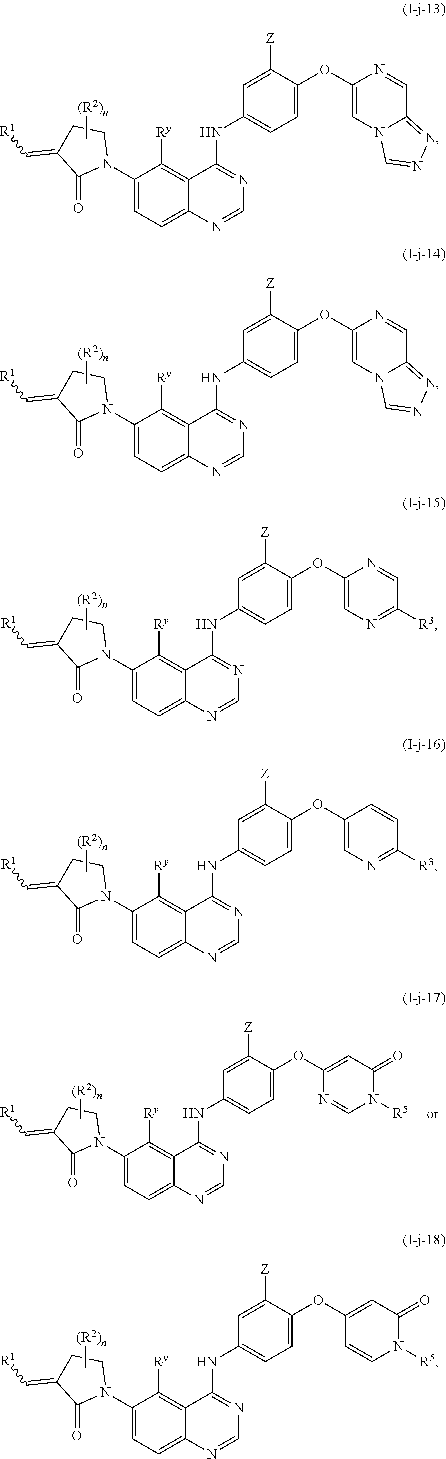

wherein R¹, R², n, R³, R⁵, R^{y}, and Z are as defined for formula (I), formula (II-a), formula (III), or formula (IV). In some embodiments, the compound is a compound of formula (I-j-1). In some embodiments, the compound is a compound of formula (I-j-2). In some embodiments, the compound is a compound of formula (I-j-3). In some embodiments, the compound is a compound of formula (I-j-4). In some embodiments, the compound is a compound of formula (I-j-5). In some embodiments, the compound is a compound of formula (I-j-6). In some embodiments, the compound is a compound of formula (I-j-7). In some embodiments, the compound is a compound of formula (I-j-8). In some embodiments, the compound is a compound of formula (I-j-9). In some embodiments, the compound is a compound of formula (I-j-10). In some embodiments, the compound is a compound of formula (I-j-11). In some embodiments, the compound is a compound of formula (I-j-12). In some embodiments, the compound is a compound of formula (I-j-13). In some embodiments, the compound is a compound of formula (I-j-14). In some embodiments, the compound is a compound of formula (I-j-15). In some embodiments, the compound is a compound of formula (I-j-16). In some embodiments, the compound is a compound of formula (I-j-17). In some embodiments, the compound is a compound of formula (I-j-18). In some embodiments, R¹ is —H. In some embodiments, R¹ is C₁-C₂ alkyl optionally substituted by —OH or —N(C₁-C₃ alkyl)(C₁-C₃ alkyl). In some embodiments, R¹ is unsubstituted C₁-C₂ alkyl. In some embodiments, R¹ is —CH₃. In some embodiments, R¹ is C₁-C₂ alkyl substituted by —N(C₁-C₂ alkyl)(C₁-C₂ alkyl). In some embodiments, R¹ is —CH₂N(CH₃)₂. In some embodiments, R¹ is C₁-C₂ alkyl substituted by —OH. In some embodiments, R¹ is —CH₂CH₂OH. In some embodiments, R² is H, —CH₃, or —CH₂CH₃. In some embodiments, R² is H. In some embodiments, R² is —CH₃. In other embodiments, wherein n is 2 and two R² groups are present on the same carbon atom, the two R² groups on the same carbon atom may be taken together with the carbon atom to which they are attached to form a C₃-C₆ spirocycloalkyl or 4- to 6-membered spiroheterocycloalkyl containing 1-2 ring heteroatoms selected from the group consisting of N, O, and S. In certain embodiments, wherein n is 2 and two R² groups are present on the same carbon atom, the two R² groups on the same carbon atom may be taken together with the carbon atom to which they are attached to form a C₃-spirocycloalkyl. In still other embodiments, wherein n is 2 and two R² groups are present vicinal carbon atoms, the two R² groups on vicinal carbon atoms may be taken together with the two carbon atoms to form a fused C₃-C₆ cycloalkyl. In certain embodiments, wherein n is 2 and two R² groups are present vicinal carbon atoms, the two R² groups on vicinal carbon atoms may be taken together with the two carbon atoms to form a fused C₃-cycloalkyl. In some embodiments, R^{y} is —H. In some embodiments, R^{y} is —F. In some embodiments of the compound of formula (I-j-15) or (I-j-16), R³ is —CH₃, —CH₂CH₃, —CHF₂, or —CD₃. In some embodiments of the compound of formula (I-j-17) or (I-j-18), R⁵ is —CH₃, —CH₂CH₃, —CHF₂, or —CD₃.

In some embodiments, the compound of formula (I-b) is a compound of formula (I-k-1), (I-k-2), (I-k-3), (I-k-4), (I-k-5), (I-k-6), (I-k-7), (I-k-8), (I-k-9), (I-k-10), (I-k-11), (I-k-12), (I-k-13), (I-k-14), (I-k-15), (I-k-16), (I-k-17), or (I-k-18):

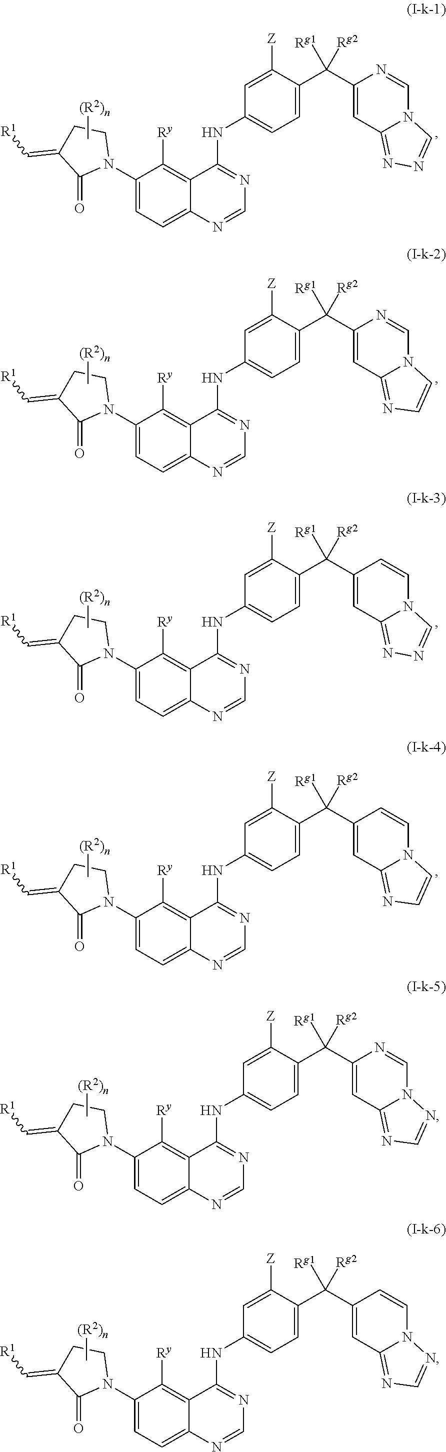

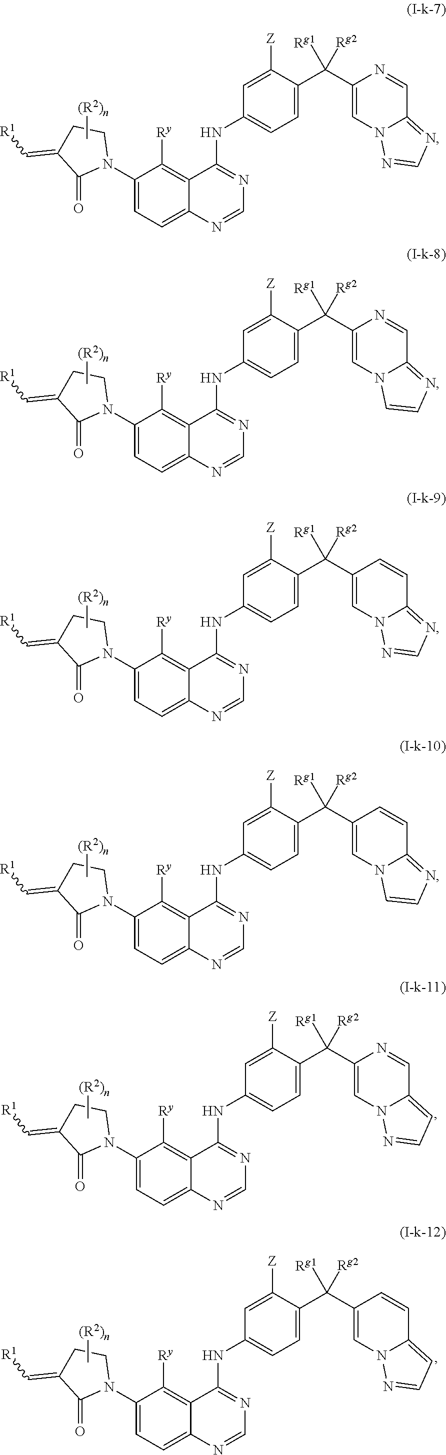

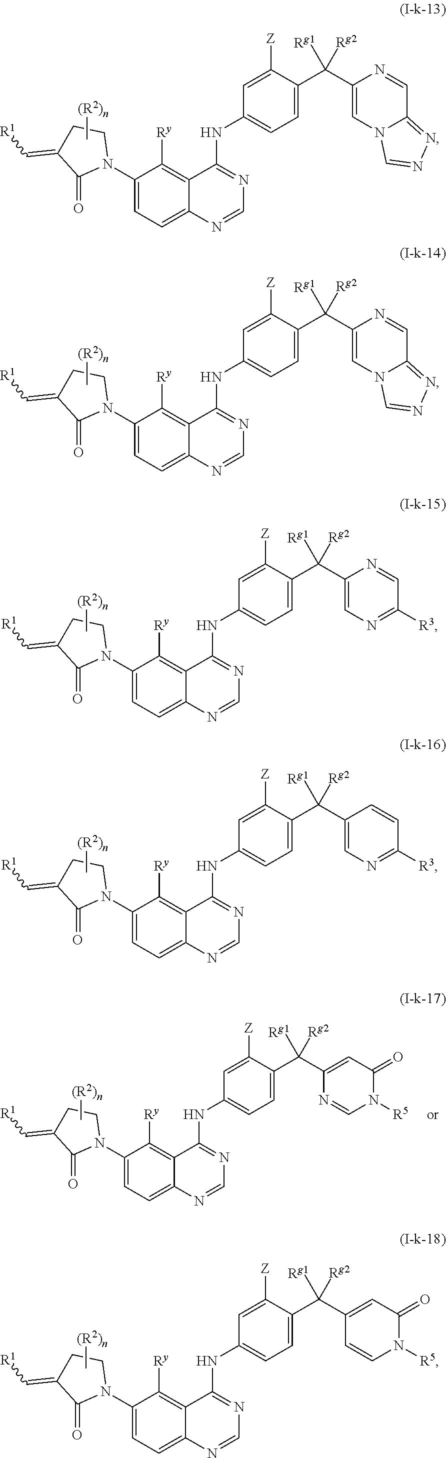

wherein R¹, R², n, R³, R⁵, R^{g1}, R^{g2}, R^{y}, and Z are as defined for formula (I), formula (II-a), formula (III), or formula (IV). In some embodiments, the compound is a compound of formula (I-k-1). In some embodiments, the compound is a compound of formula (I-k-2). In some embodiments, the compound is a compound of formula (I-k-3). In some embodiments, the compound is a compound of formula (I-k-4). In some embodiments, the compound is a compound of formula (I-k-5). In some embodiments, the compound is a compound of formula (I-k-6). In some embodiments, the compound is a compound of formula (I-k-7). In some embodiments, the compound is a compound of formula (I-k-8). In some embodiments, the compound is a compound of formula (I-k-9). In some embodiments, the compound is a compound of formula (I-k-10). In some embodiments, the compound is a compound of formula (I-k-11). In some embodiments, the compound is a compound of formula (I-k-12). In some embodiments, the compound is a compound of formula (I-k-13). In some embodiments, the compound is a compound of formula (I-k-14). In some embodiments, the compound is a compound of formula (I-k-15). In some embodiments, the compound is a compound of formula (I-k-16). In some embodiments, the compound is a compound of formula (I-k-17). In some embodiments, the compound is a compound of formula (I-k-18). In some embodiments, R¹ is —H. In some embodiments, R¹ is C₁-C₂ alkyl optionally substituted by —OH or —N(C₁-C₃ alkyl)(C₁-C₃ alkyl). In some embodiments, R¹ is unsubstituted C₁-C₂ alkyl. In some embodiments, R¹ is —CH₃. In some embodiments, R¹ is C₁-C₂ alkyl substituted by —N(C₁-C₂ alkyl)(C₁-C₂ alkyl). In some embodiments, R¹ is —CH₂N(CH₃)₂. In some embodiments, R¹ is C₁-C₂ alkyl substituted by —OH. In some embodiments, R¹ is —CH₂CH₂OH. In some embodiments, R² is —H, —CH₃, or —CH₂CH₃. In some embodiments, R² is —H. In some embodiments, R² is —CH₃.

In other embodiments, wherein n is 2 and two R² groups are present on the same carbon atom, the two R² groups on the same carbon atom may be taken together with the carbon atom to which they are attached to form a C₃-C₆ spirocycloalkyl or 4- to 6-membered spiroheterocycloalkyl containing 1-2 ring heteroatoms selected from the group consisting of N, O, and S. In certain embodiments, wherein n is 2 and two R² groups are present on the same carbon atom, the two R² groups on the same carbon atom may be taken together with the carbon atom to which they are attached to form a C₃-spirocycloalkyl. In still other embodiments, wherein n is 2 and two R² groups are present vicinal carbon atoms, the two R² groups on vicinal carbon atoms may be taken together with the two carbon atoms to form a fused C₃-C₆ cycloalkyl. In certain embodiments, wherein n is 2 and two R² groups are present vicinal carbon atoms, the two R² groups on vicinal carbon atoms may be taken together with the two carbon atoms to form a fused C₃-cycloalkyl. In some embodiments, R^{y} is —H. In some embodiments, R^{y} is —F. In some embodiments of the compound of formula (I-k-15) or (I-k-16), R³ is —CH₃, —CH₂CH₃, —CHF₂, or —CD₃. In some embodiments of the compound of formula (I-k-17) or (I-k-18), R⁵ is —CH₃, —CH₂CH₃, —CHF₂, or —CD₃.

In some embodiments, the compound of formula (I-c) is a compound of formula (I-l-1), (I-l-2), (I-l-3), (I-l-4), (I-l-5), (I-l-6), (I-l-7), (I-l-8), (I-l-9), (I-l-10), (I-l-11), (I-l-12), (I-l-13), (I-l-14), (I-l-15), (I-l-16), (I-l-17), or (I-l-18):

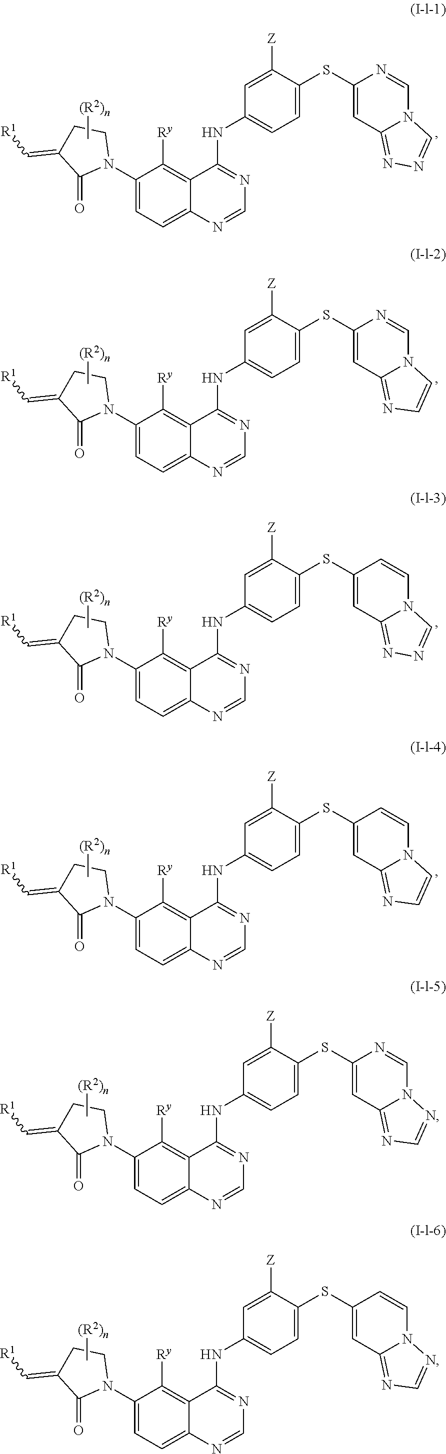

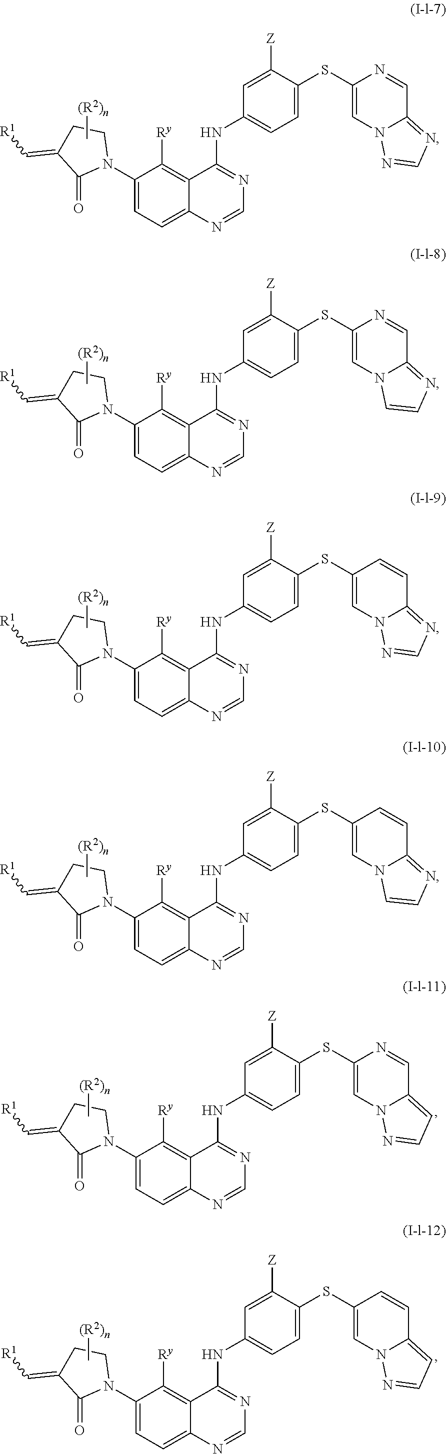

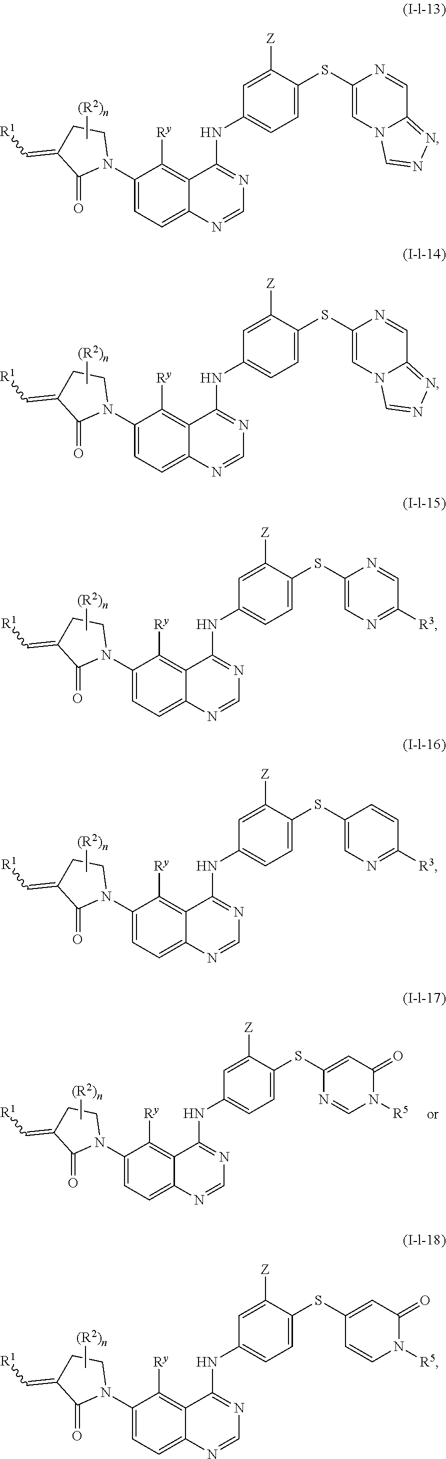

wherein R¹, R², n, R³, R⁵, R^{y}, and Z are as defined for formula (I), formula (II-a), formula (III), or formula (IV). In some embodiments, the compound is a compound of formula (I-l-1). In some embodiments, the compound is a compound of formula (I-l-2). In some embodiments, the compound is a compound of formula (I-l-3). In some embodiments, the compound is a compound of formula (I-l-4). In some embodiments, the compound is a compound of formula (I-l-5). In some embodiments, the compound is a compound of formula (I-l-6). In some embodiments, the compound is a compound of formula (I-l-7). In some embodiments, the compound is a compound of formula (I-l-8). In some embodiments, the compound is a compound of formula (I-l-9). In some embodiments, the compound is a compound of formula (I-l-10). In some embodiments, the compound is a compound of formula (I-l-11). In some embodiments, the compound is a compound of formula (I-l-12). In some embodiments, the compound is a compound of formula (I-l-13). In some embodiments, the compound is a compound of formula (I-l-14). In some embodiments, the compound is a compound of formula (I-l-15). In some embodiments, the compound is a compound of formula (I-l-16). In some embodiments, the compound is a compound of formula (I-l-17). In some embodiments, the compound is a compound of formula (I-l-18). In some embodiments, R¹ is —H. In some embodiments, R¹ is C₁-C₂ alkyl optionally substituted by —OH or —N(C₁-C₃ alkyl)(C₁-C₃ alkyl). In some embodiments, R¹ is unsubstituted C₁-C₂ alkyl. In some embodiments, R¹ is —CH₃. In some embodiments, R¹ is C₁-C₂ alkyl substituted by —N(C₁-C₂ alkyl)(C₁-C₂ alkyl). In some embodiments, R¹ is —CH₂N(CH₃)₂. In some embodiments, R¹ is C₁-C₂ alkyl substituted by —OH. In some embodiments, R¹ is —CH₂CH₂OH. In some embodiments, R² is —H, —CH₃, or —CH₂CH₃. In some embodiments, R² is —H. In some embodiments, R² is —CH₃. In other embodiments, wherein n is 2 and two R² groups are present on the same carbon atom, the two R² groups on the same carbon atom may be taken together with the carbon atom to which they are attached to form a C₃-C₆ spirocycloalkyl or 4- to 6-membered spiroheterocycloalkyl containing 1-2 ring heteroatoms selected from the group consisting of N, O, and S. In certain embodiments, wherein n is 2 and two R² groups are present on the same carbon atom, the two R² groups on the same carbon atom may be taken together with the carbon atom to which they are attached to form a C₃-spirocycloalkyl. In still other embodiments, wherein n is 2 and two R² groups are present vicinal carbon atoms, the two R² groups on vicinal carbon atoms may be taken together with the two carbon atoms to form a fused C₃-C₆ cycloalkyl. In certain embodiments, wherein n is 2 and two R² groups are present vicinal carbon atoms, the two R² groups on vicinal carbon atoms may be taken together with the two carbon atoms to form a fused C₃-cycloalkyl. In some embodiments, R^{3′} is —H. In some embodiments, R^{3′} is —F. In some embodiments of the compound of formula (I-l-15) or (I-l-16), R³ is —CH₃, —CH₂CH₃, —CHF₂, or —CD₃. In some embodiments of the compound of formula (I-l-17) or (I-l-18), R⁵ is —CH₃, —CH₂CH₃, —CHF₂, or —CD₃.

In some embodiments, provided is a compound of formula (I), a compound of formula (II-a), compound of formula (II-b), compound of formula (II-c), compound of formula (II-d), a compound of formula (III), or a compound of formula (IV) selected from the compounds in Table 1, or pharmaceutically acceptable salt, solvate, hydrate, or co-crystal thereof, or a mixture of any of the foregoing. Although certain compounds described in Table 1 may be presented as specific stereoisomers and/or in a non-stereochemical form, it is understood that any or all stereochemical forms, including any enantiomeric or diastereomeric forms, and any tautomers or other forms of any of the compounds of Table 1 are herein described.

TABLE 1

| Cmpd | | Cmpd | |
|---|---|---|---|
| No. | Structure | No. | Structure |
| 1 | 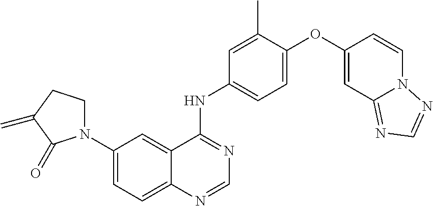 [SMILES: C=C1CCN(c2ccc3ncnc(Nc4ccc(Oc5ccn6ncnc6c5)c(C)c4)c3c2)C1=O] | | |
| 2 | 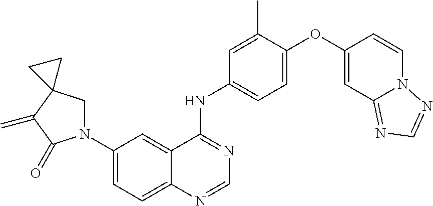 [SMILES: C=C1C(=O)N(c2ccc3ncnc(Nc4ccc(Oc5ccn6ncnc6c5)c(C)c4)c3c2)CC12CC2] | | |
| 3 | 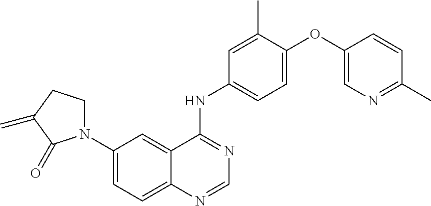 | | |
| 4 | 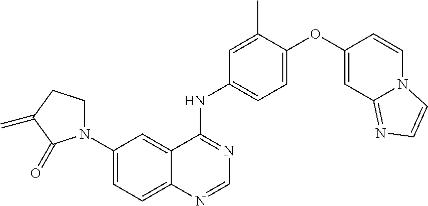 [SMILES: C=C1CCN(c2ccc3ncnc(Nc4ccc(Oc5ccn6ccnc6c5)c(C)c4)c3c2)C1=O] | | |
| 5 | 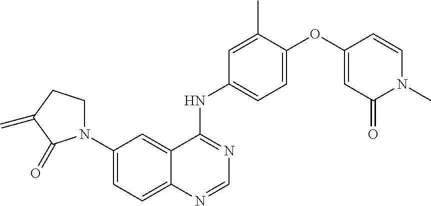 | | |
| 6 | 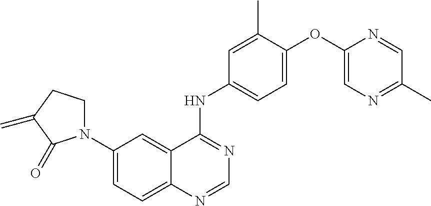 | | |
| 7 | 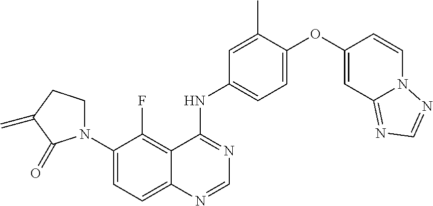 [SMILES: C=C1CCN(c2ccc3ncnc(Nc4ccc(Oc5ccn6ncnc6c5)c(C)c4)c3c2F)C1=O] | | |
| 8 | 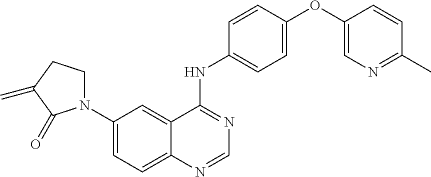 | | |
| 9 | 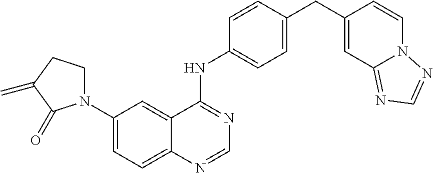 [SMILES: C=C1CCN(c2ccc3ncnc(Nc4ccc(Cc5ccn6ncnc6c5)cc4)c3c2)C1=O] | | |
| 10 | 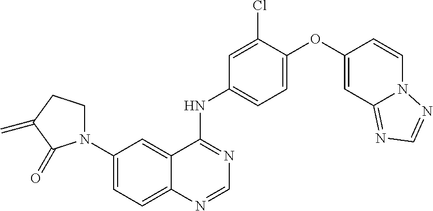 [SMILES: C=C1CCN(c2ccc3ncnc(Nc4ccc(Oc5ccn6ncnc6c5)c(Cl)c4)c3c2)C1=O] | | |
| 11 | 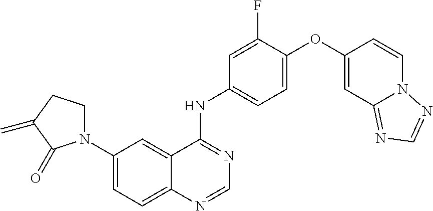 [SMILES: C=C1CCN(c2ccc3ncnc(Nc4ccc(Oc5ccn6ncnc6c5)c(F)c4)c3c2)C1=O] | | |
| 12 | 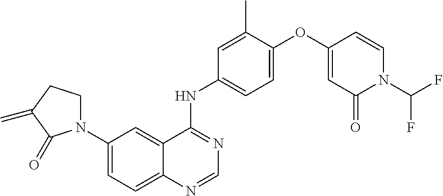 [SMILES: C=C1CCN(c2ccc3ncnc(Nc4ccc(Oc5ccn(C(F)F)c(=O)c5)c(C)c4)c3c2)C1=O] | | |
| 13 | 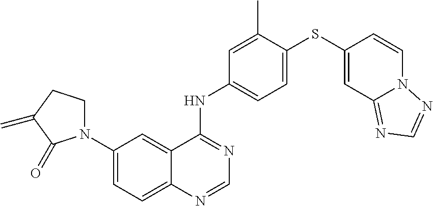 [SMILES: C=C1CCN(c2ccc3ncnc(Nc4ccc(Sc5ccn6ncnc6c5)c(C)c4)c3c2)C1=O] | | |
| 14 | 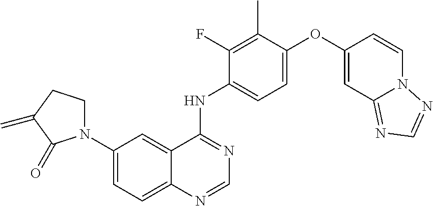 [SMILES: C=C1CCN(c2ccc3ncnc(Nc4ccc(Oc5ccn6ncnc6c5)c(C)c4F)c3c2)C1=O] | | |
| 15 | 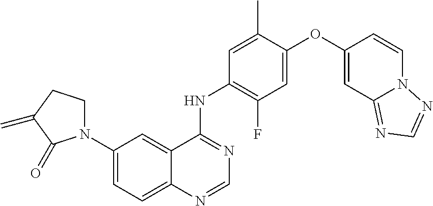 [SMILES: C=C1CCN(c2ccc3ncnc(Nc4cc(C)c(Oc5ccn6ncnc6c5)cc4F)c3c2)C1=O] | | |
| 16 | 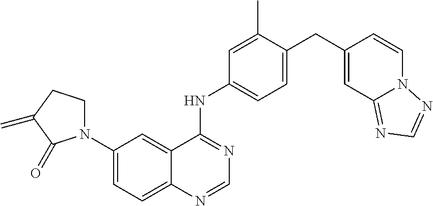 [SMILES: C=C1CCN(c2ccc3ncnc(Nc4ccc(Cc5ccn6ncnc6c5)c(C)c4)c3c2)C1=O] | | |
| 17 | 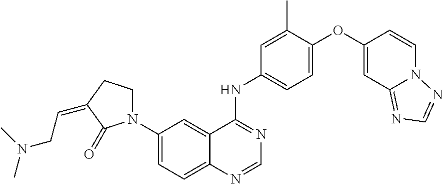 [SMILES: Cc1cc(Nc2ncnc3ccc(N4CC/C(=C/CN(C)C)C4=O)cc23)ccc1Oc1ccn2ncnc2c1] | | |
| 18 | 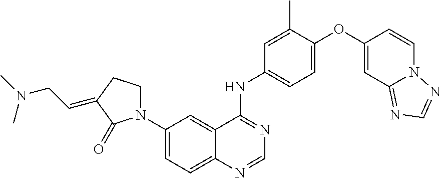 [SMILES: Cc1cc(Nc2ncnc3ccc(N4CC/C(=C\CN(C)C)C4=O)cc23)ccc1Oc1ccn2ncnc2c1] | | |
| 19 | 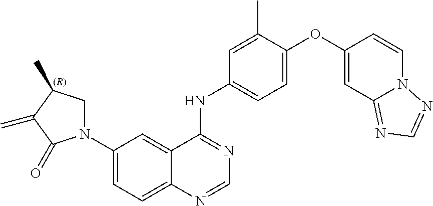 | 19-1 | 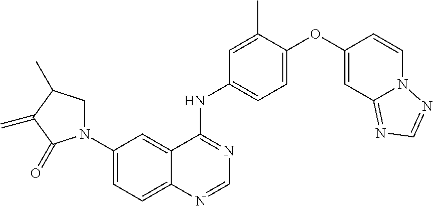 [SMILES: C=C1C(=O)N(c2ccc3ncnc(Nc4ccc(Oc5ccn6ncnc6c5)c(C)c4)c3c2)CC1C] |
| 20 | 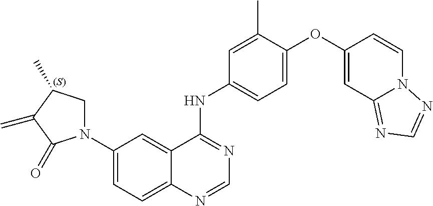 | 19-1 | 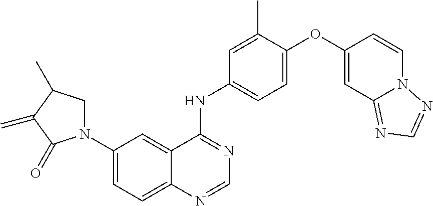 [SMILES: C=C1C(=O)N(c2ccc3ncnc(Nc4ccc(Oc5ccn6ncnc6c5)c(C)c4)c3c2)CC1C] |
| 21 | 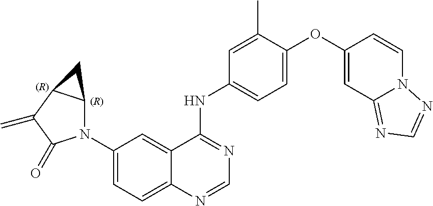 | 21-1 | 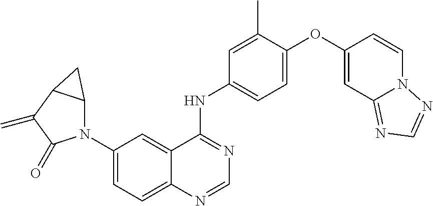 [SMILES: C=C1C(=O)N(c2ccc3ncnc(Nc4ccc(Oc5ccn6ncnc6c5)c(C)c4)c3c2)C2CC12] |
| 22 | 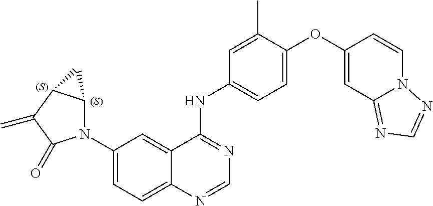 | 21-1 | 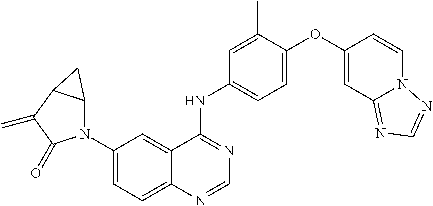 [SMILES: C=C1C(=O)N(c2ccc3ncnc(Nc4ccc(Oc5ccn6ncnc6c5)c(C)c4)c3c2)C2CC12] |
| 23 | 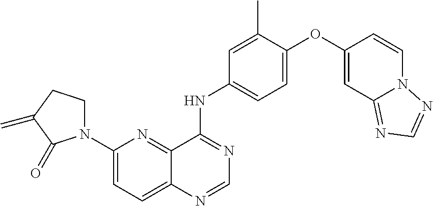 [SMILES: C=C1CCN(c2ccc3ncnc(Nc4ccc(Oc5ccn6ncnc6c5)c(C)c4)c3n2)C1=O] | | |
| 24 | 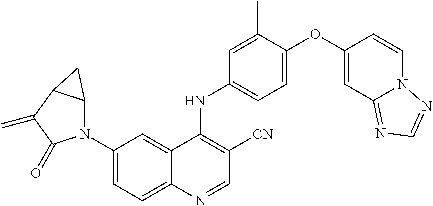 [SMILES: C=C1C(=O)N(c2ccc3ncc(C#N)c(Nc4ccc(Oc5ccn6ncnc6c5)c(C)c4)c3c2)C2CC12] | | |
| 25 | 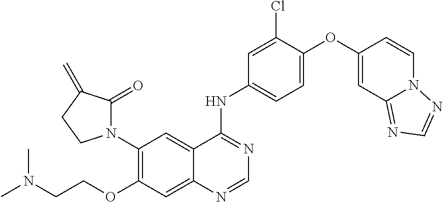 [SMILES: C=C1CCN(c2cc3c(Nc4ccc(Oc5ccn6ncnc6c5)c(Cl)c4)ncnc3cc2OCCN(C)C)C1=O] | | |
| 26 | 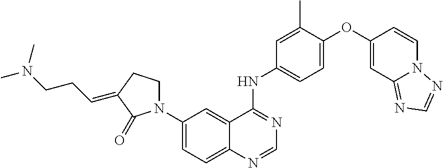 [SMILES: Cc1cc(Nc2ncnc3ccc(N4CC/C(=C\CCN(C)C)C4=O)cc23)ccc1Oc1ccn2ncnc2c1] | | |
| 27 | 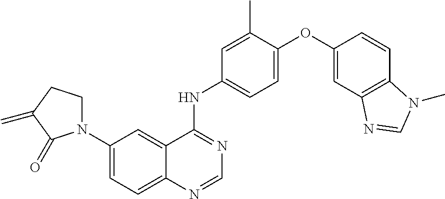 | | |
| 28 | 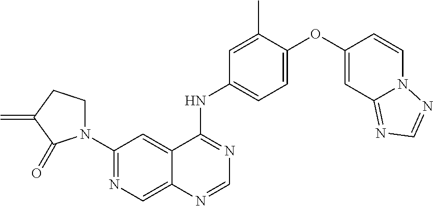 [SMILES: C=C1CCN(c2cc3c(Nc4ccc(Oc5ccn6ncnc6c5)c(C)c4)ncnc3cn2)C1=O] | | |
| 29 | 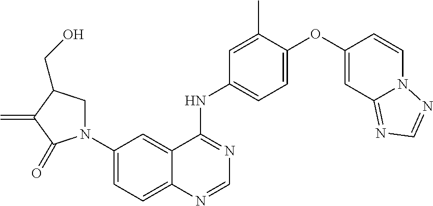 [SMILES: C=C1C(=O)N(c2ccc3ncnc(Nc4ccc(Oc5ccn6ncnc6c5)c(C)c4)c3c2)CC1CO] | | |
| 30 | 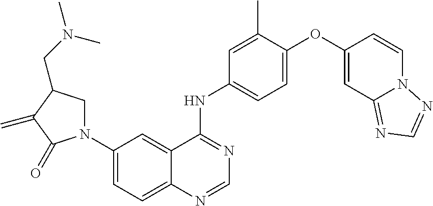 [SMILES: C=C1C(=O)N(c2ccc3ncnc(Nc4ccc(Oc5ccn6ncnc6c5)c(C)c4)c3c2)CC1CN(C)C] | | |
| 31 | 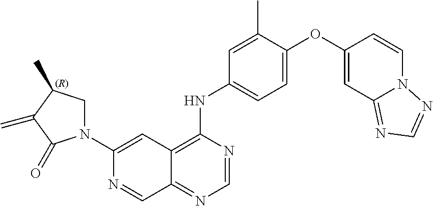 | 31-1 | 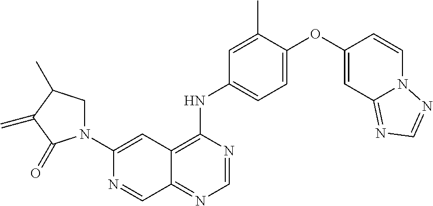 [SMILES: C=C1C(=O)N(c2cc3c(Nc4ccc(Oc5ccn6ncnc6c5)c(C)c4)ncnc3cn2)CC1C] |
| 32 | 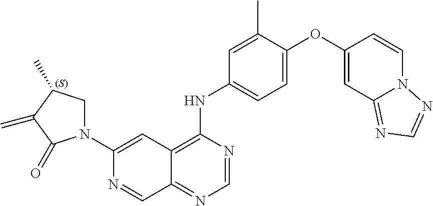 | 31-1 | 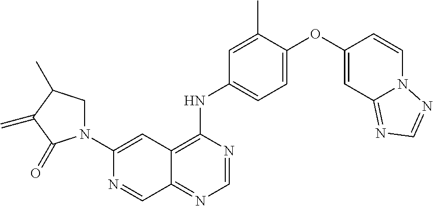 [SMILES: C=C1C(=O)N(c2cc3c(Nc4ccc(Oc5ccn6ncnc6c5)c(C)c4)ncnc3cn2)CC1C] |
| 33 | 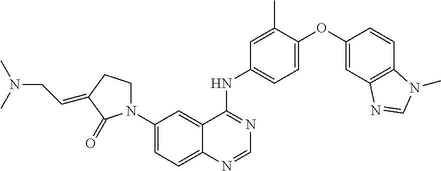 | | |
| 34 | 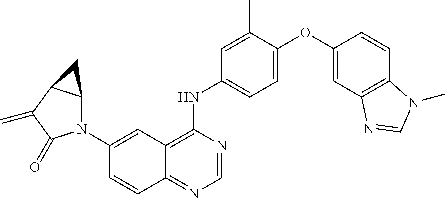 | 34-1 | 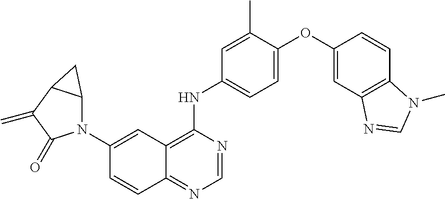 |
| 35 | 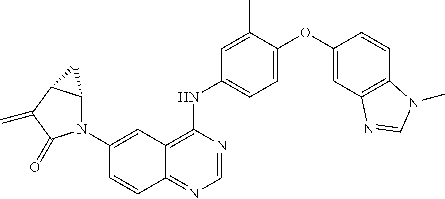 | 34-1 | 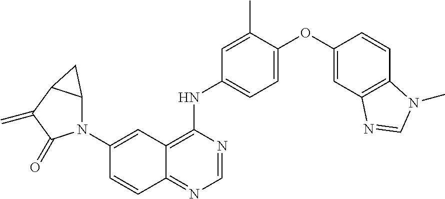 |
| 36 | 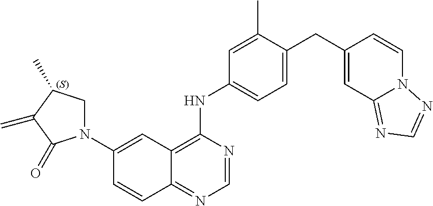 | 36-1 | 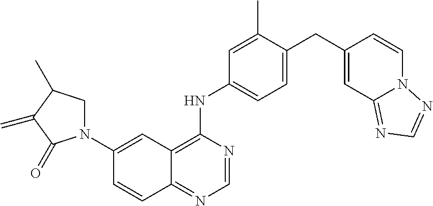 [SMILES: C=C1C(=O)N(c2ccc3ncnc(Nc4ccc(Cc5ccn6ncnc6c5)c(C)c4)c3c2)CC1C] |
| 37 | 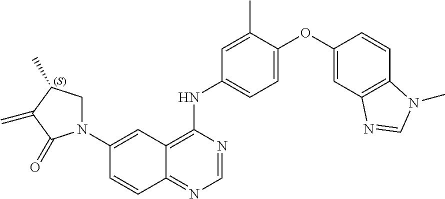 | 37-1 | 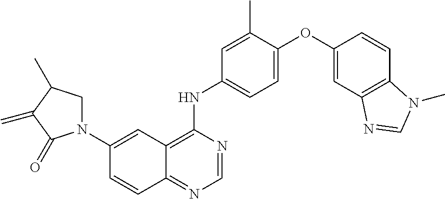 |
| 38 | 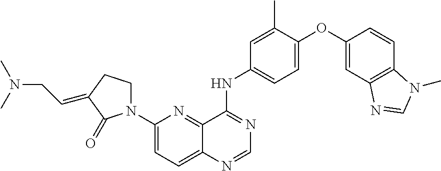 | | |
| 39 | 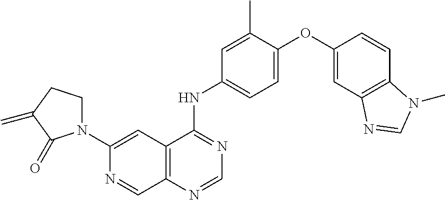 | | |
| 40 | 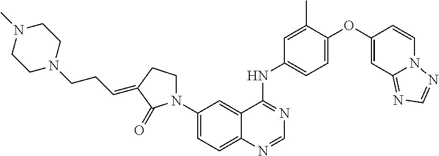 [SMILES: Cc1cc(Nc2ncnc3ccc(N4CC/C(=C\CCN5CCN(C)CC5)C4=O)cc23)ccc1Oc1ccn2ncnc2c1] | | |
| 41 | 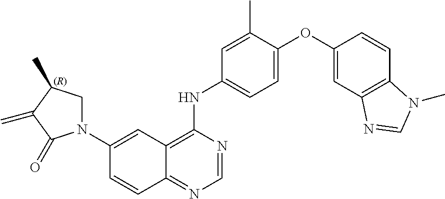 | 37-1 | 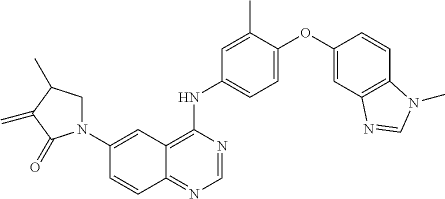 |
| 42 | 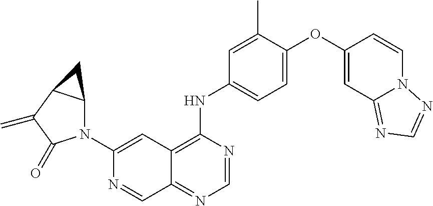 [SMILES: C=C1C(=O)N(c2cc3c(Nc4ccc(Oc5ccn6ncnc6c5)c(C)c4)ncnc3cn2)[C@@H]2C[C@H]12] | 42-1 | 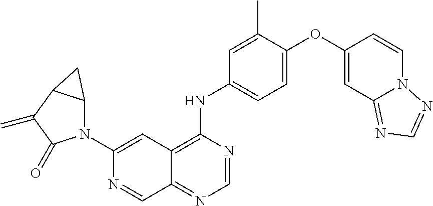 [SMILES: C=C1C(=O)N(c2cc3c(Nc4ccc(Oc5ccn6ncnc6c5)c(C)c4)ncnc3cn2)C2CC12] |
| 43 | 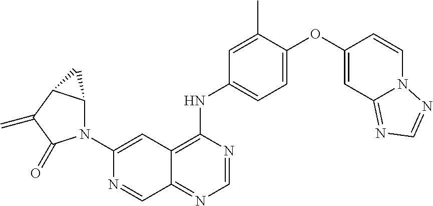 [SMILES: C=C1C(=O)N(c2cc3c(Nc4ccc(Oc5ccn6ncnc6c5)c(C)c4)ncnc3cn2)[C@H]2C[C@@H]12] | 42-1 | 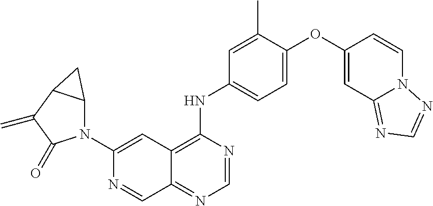 [SMILES: C=C1C(=O)N(c2cc3c(Nc4ccc(Oc5ccn6ncnc6c5)c(C)c4)ncnc3cn2)C2CC12] |
| 44 | 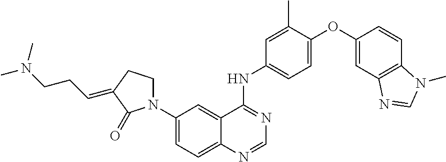 | | |
| 45 | 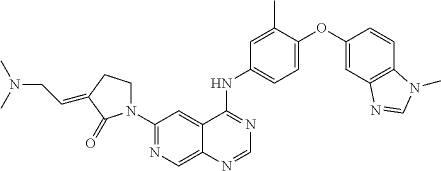 | | |
| 46 | 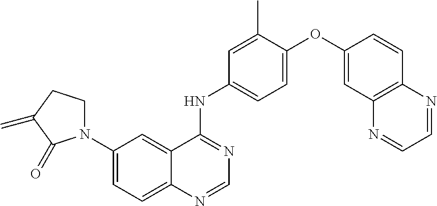 [SMILES: C=C1CCN(c2ccc3ncnc(Nc4ccc(Oc5ccc6nccnc6c5)c(C)c4)c3c2)C1=O] | | |
| 47 | 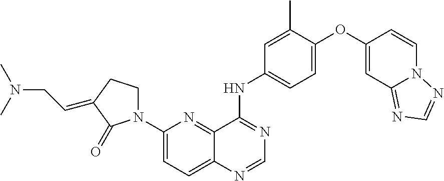 [SMILES: Cc1cc(Nc2ncnc3ccc(N4CC/C(=C\CN(C)C)C4=O)nc23)ccc1Oc1ccn2ncnc2c1] | | |
| 48 | 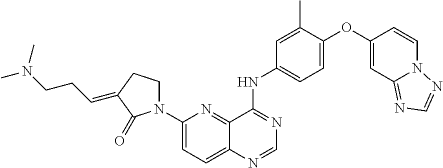 [SMILES: Cc1cc(Nc2ncnc3ccc(N4CC/C(=C\CCN(C)C)C4=O)nc23)ccc1Oc1ccn2ncnc2c1] | | |
| 49 | 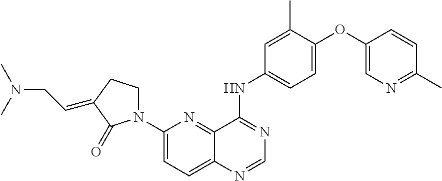 | | |
| 50 | 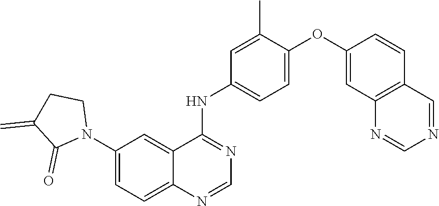 [SMILES: C=C1CCN(c2ccc3ncnc(Nc4ccc(Oc5ccc6cncnc6c5)c(C)c4)c3c2)C1=O] | | |
| 51 | 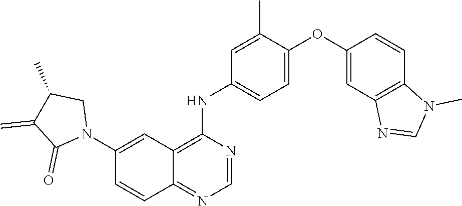 | 51-1 | 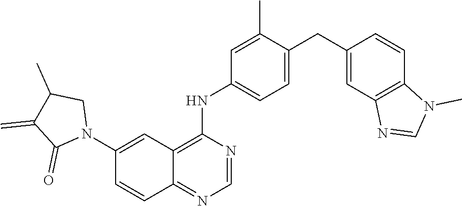 |
| 52 | 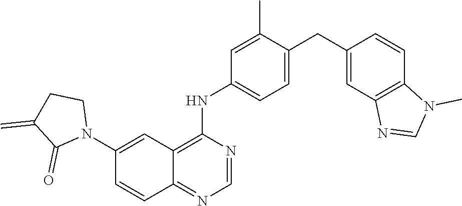 | | |
| 53 | 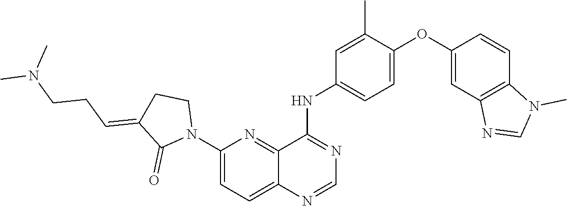 | | |
| 54 | 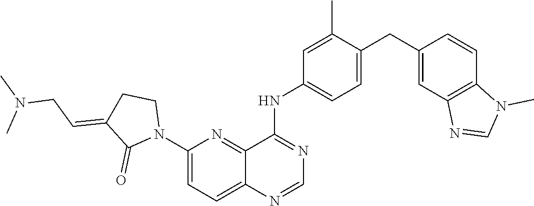 | | |
| 55 | 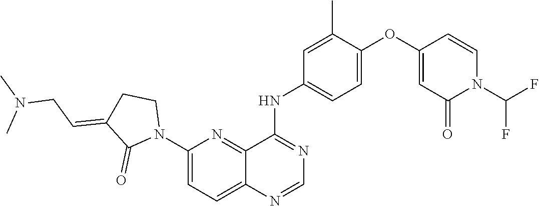 [SMILES: Cc1cc(Nc2ncnc3ccc(N4CC/C(=C\CN(C)C)C4=O)nc23)ccc1Oc1ccn(C(F)F)c(=O)c1] | | |
| 56 | 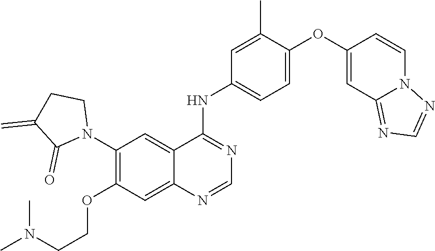 [SMILES: C=C1CCN(c2cc3c(Nc4ccc(Oc5ccn6ncnc6c5)c(C)c4)ncnc3cc2OCCN(C)C)C1=O] | | |
| 57 | 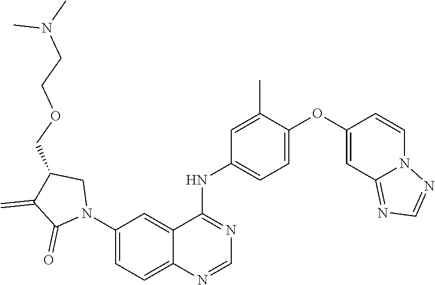 [SMILES: C=C1C(=O)N(c2ccc3ncnc(Nc4ccc(Oc5ccn6ncnc6c5)c(C)c4)c3c2)C[C@H]1COCCN(C)C] | 57-1 | 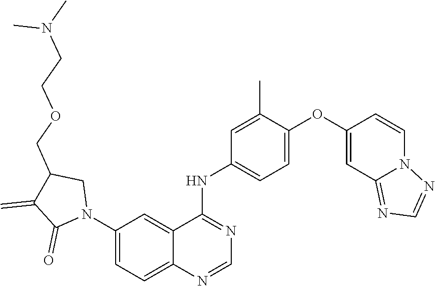 [SMILES: C=C1C(=O)N(c2ccc3ncnc(Nc4ccc(Oc5ccn6ncnc6c5)c(C)c4)c3c2)CC1COCCN(C)C] |
| 58 | 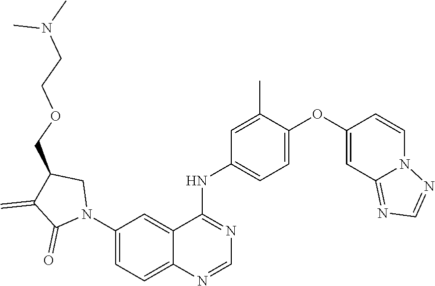 [SMILES: C=C1C(=O)N(c2ccc3ncnc(Nc4ccc(Oc5ccn6ncnc6c5)c(C)c4)c3c2)C[C@@H]1COCCN(C)C] | 57-1 | 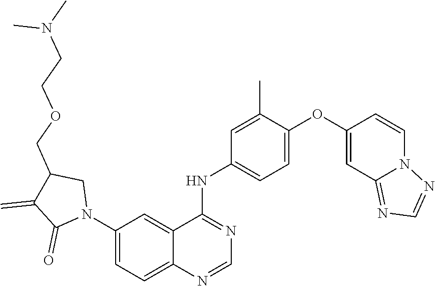 [SMILES: C=C1C(=O)N(c2ccc3ncnc(Nc4ccc(Oc5ccn6ncnc6c5)c(C)c4)c3c2)CC1COCCN(C)C] |
| 59 | 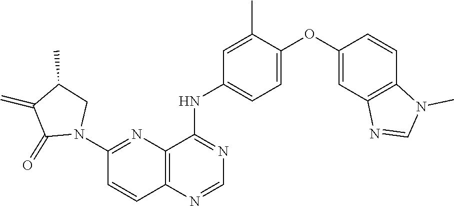 | 59-1 | 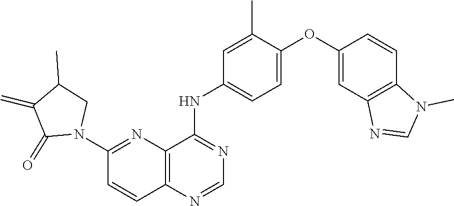 |
| 60 | 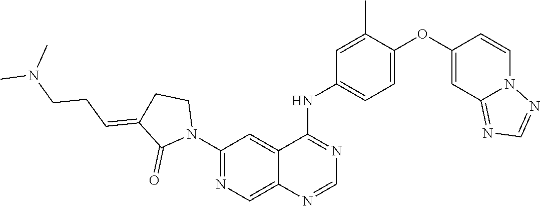 [SMILES: Cc1cc(Nc2ncnc3cnc(N4CC/C(=C\CCN(C)C)C4=O)cc23)ccc1Oc1ccn2ncnc2c1] | | |
| 61 | 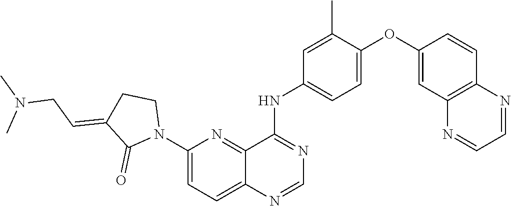 [SMILES: Cc1cc(Nc2ncnc3ccc(N4CC/C(=C\CN(C)C)C4=O)nc23)ccc1Oc1ccc2nccnc2c1] | | |
| 62 | 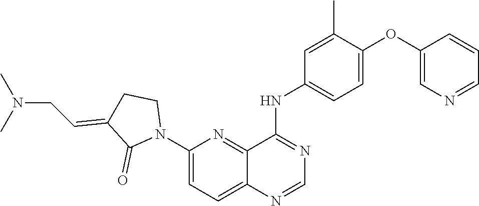 [SMILES: Cc1cc(Nc2ncnc3ccc(N4CC/C(=C\CN(C)C)C4=O)nc23)ccc1Oc1cccnc1] | | |
| 63 | 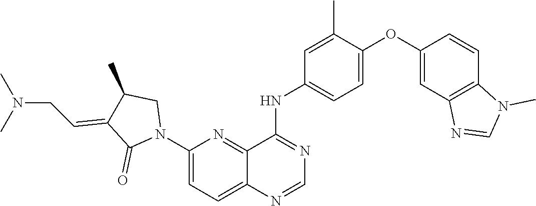 | 63-1 | 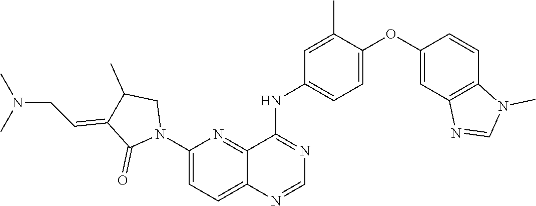 |
| 64 | 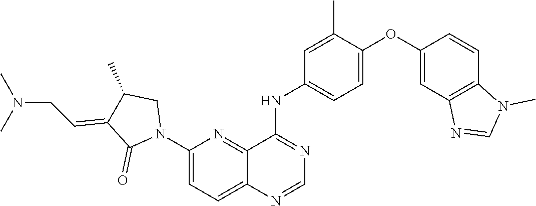 | 63-1 | 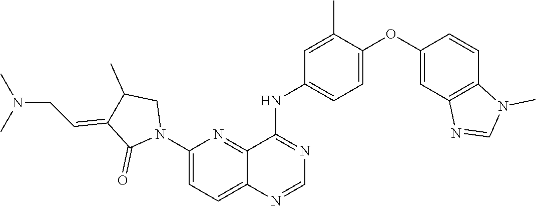 |
| 65 | 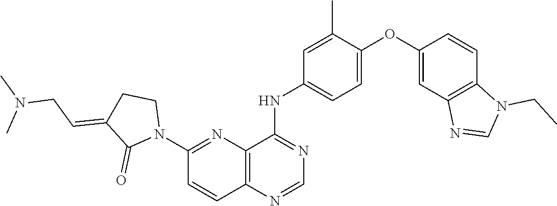 [SMILES: CCn1cnc2cc(Oc3ccc(Nc4ncnc5ccc(N6CC/C(=C\CN(C)C)C6=O)nc45)cc3C)ccc21] | | |
| 66 | 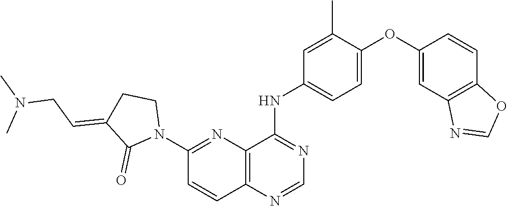 [SMILES: Cc1cc(Nc2ncnc3ccc(N4CC/C(=C\CN(C)C)C4=O)nc23)ccc1Oc1ccc2ocnc2c1] | | |
| 67 | 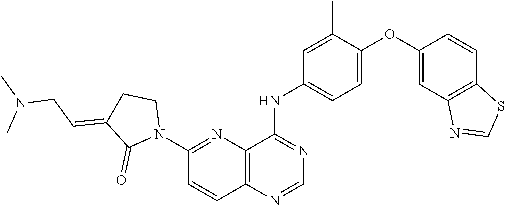 [SMILES: Cc1cc(Nc2ncnc3ccc(N4CC/C(=C\CN(C)C)C4=O)nc23)ccc1Oc1ccc2scnc2c1] | | |
| 68 | 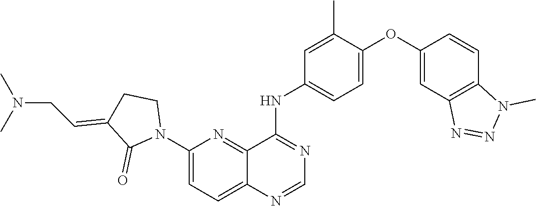 | | |
| 69 | 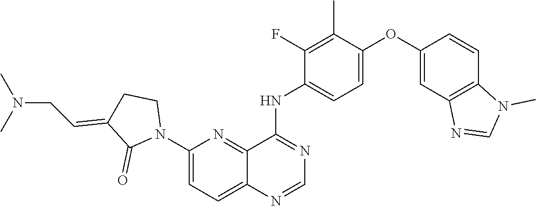 | | |
| 70 | 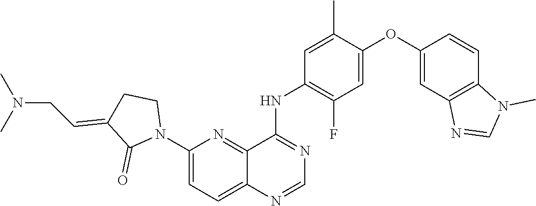 | | |
| 71 | 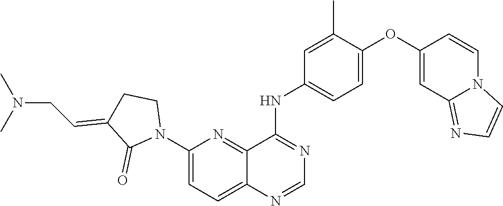 [SMILES: Cc1cc(Nc2ncnc3ccc(N4CC/C(=C\CN(C)C)C4=O)nc23)ccc1Oc1ccn2ccnc2c1] | | |
| 72 | 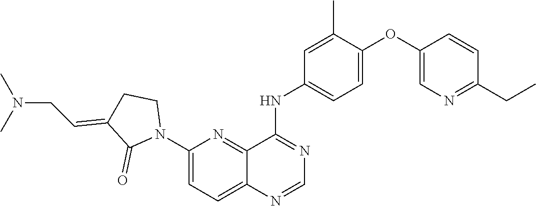 [SMILES: CCc1ccc(Oc2ccc(Nc3ncnc4ccc(N5CC/C(=C\CN(C)C)C5=O)nc34)cc2C)cn1] | | |
| 73 | 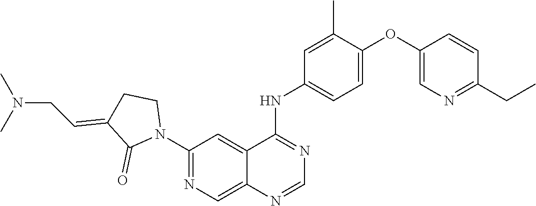 [SMILES: CCc1ccc(Oc2ccc(Nc3ncnc4cnc(N5CC/C(=C\CN(C)C)C5=O)cc34)cc2C)cn1] | | |
| 74 | 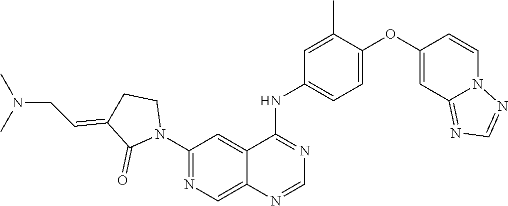 [SMILES: Cc1cc(Nc2ncnc3cnc(N4CC/C(=C\CN(C)C)C4=O)cc23)ccc1Oc1ccn2ncnc2c1] | | |
| 75 | 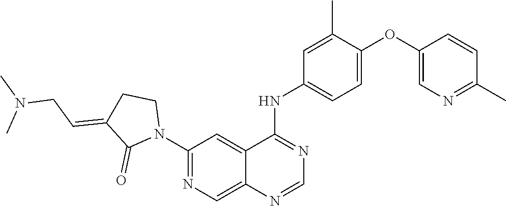 | | |
| 76 | 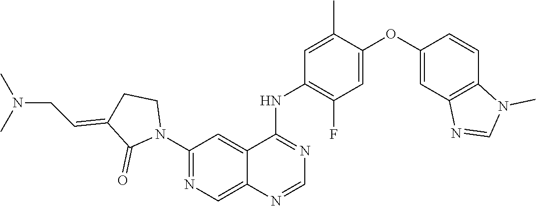 | | |
| 77 | 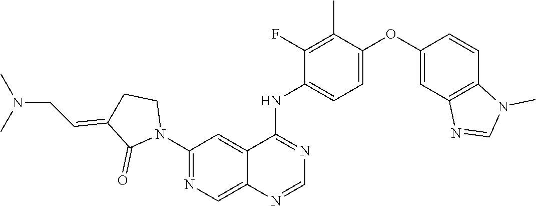 | | |
| 78 | 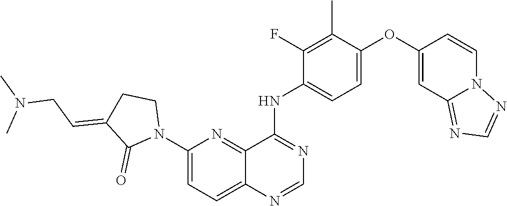 [SMILES: Cc1c(Oc2ccn3ncnc3c2)ccc(Nc2ncnc3ccc(N4CC/C(=C\CN(C)C)C4=O)nc23)c1F] | | |
| 79 | 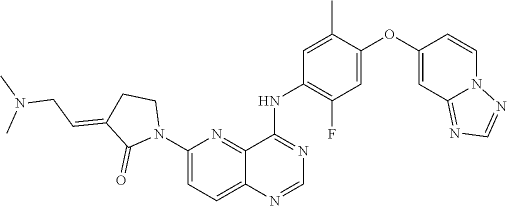 [SMILES: Cc1cc(Nc2ncnc3ccc(N4CC/C(=C\CN(C)C)C4=O)nc23)c(F)cc1Oc1ccn2ncnc2c1] | | |
| 80 | 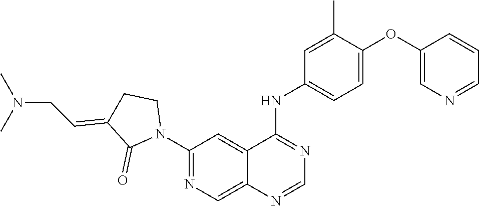 [SMILES: Cc1cc(Nc2ncnc3cnc(N4CC/C(=C\CN(C)C)C4=O)cc23)ccc1Oc1cccnc1] | | |
| 81 | 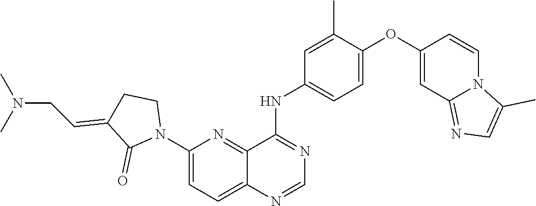 | | |
| 82 | 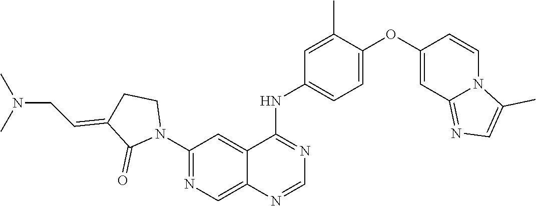 | | |
| 83 | 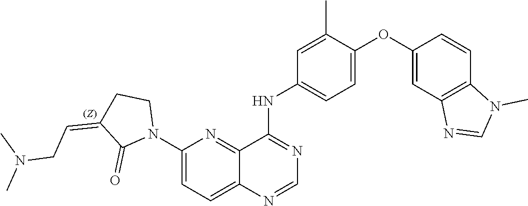 | | |
| 84 | 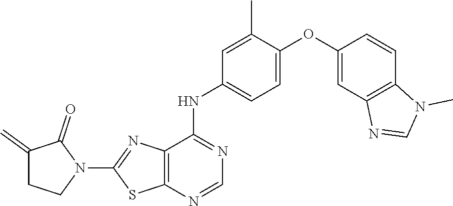 | | |
| 85 | 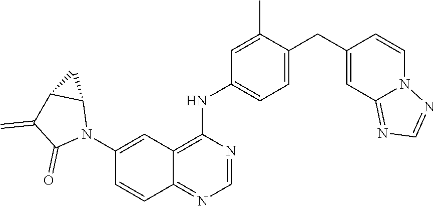 [SMILES: C=C1C(=O)N(c2ccc3ncnc(Nc4ccc(Cc5ccn6ncnc6c5)c(C)c4)c3c2)[C@H]2C[C@@H]12] | 85-1 | 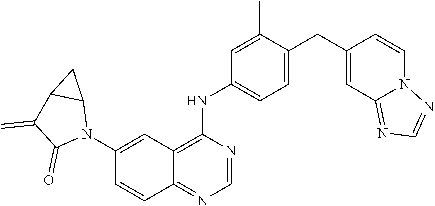 [SMILES: C=C1C(=O)N(c2ccc3ncnc(Nc4ccc(Cc5ccn6ncnc6c5)c(C)c4)c3c2)C2CC12] |
| 86 | 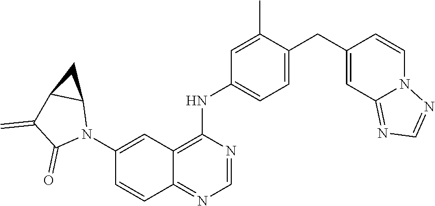 [SMILES: C=C1C(=O)N(c2ccc3ncnc(Nc4ccc(Cc5ccn6ncnc6c5)c(C)c4)c3c2)[C@@H]2C[C@H]12] | 85-1 | 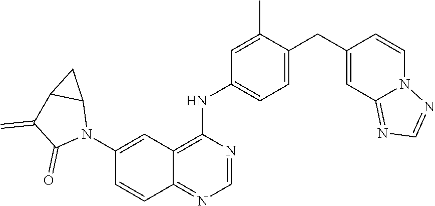 [SMILES: C=C1C(=O)N(c2ccc3ncnc(Nc4ccc(Cc5ccn6ncnc6c5)c(C)c4)c3c2)C2CC12] |
| 87 | 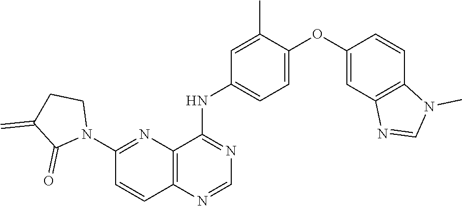 | | |
| 88 | 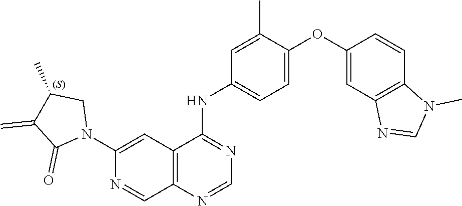 | 88-1 | 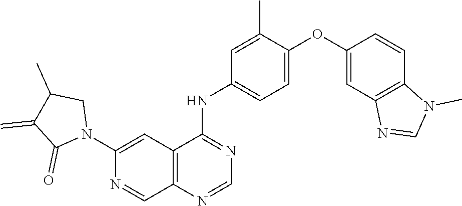 |
| 89 | 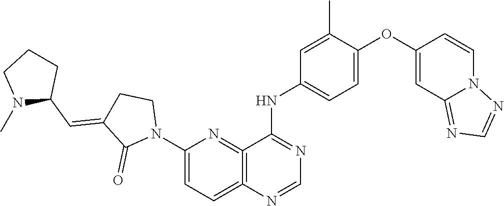 | 89-1 | 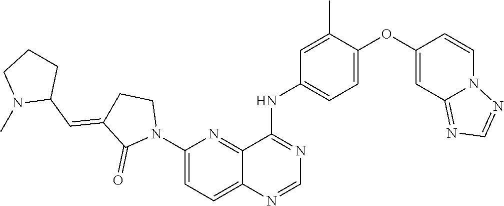 [SMILES: Cc1cc(Nc2ncnc3ccc(N4CC/C(=C\C5CCCN5C)C4=O)nc23)ccc1Oc1ccn2ncnc2c1] |
| 90 | 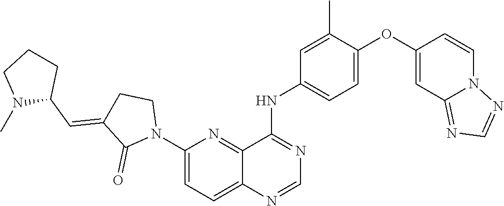 [SMILES: Cc1cc(Nc2ncnc3ccc(N4CC/C(=C\[C@H]5CCCN5C)C4=O)nc23)ccc1Oc1ccn2ncnc2c1] | 89-1 | 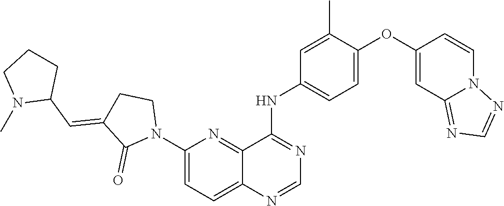 [SMILES: Cc1cc(Nc2ncnc3ccc(N4CC/C(=C\C5CCCN5C)C4=O)nc23)ccc1Oc1ccn2ncnc2c1] |
| 91 | 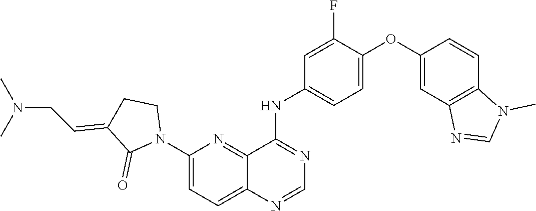 | | |
| 92 | 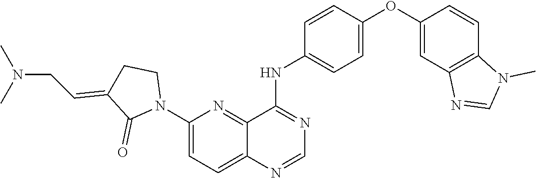 | | |
| 93 | 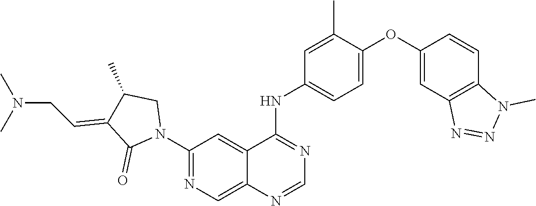 | 93-1 | 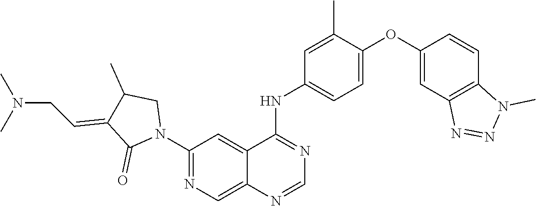 |
| 94 | 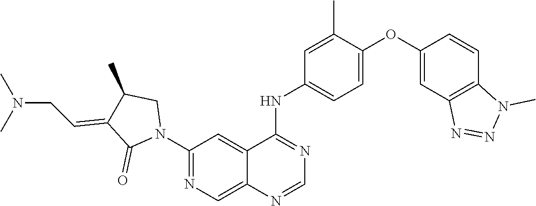 | 93-1 | 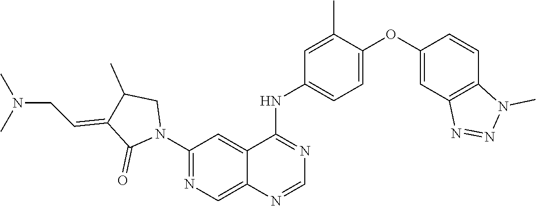 |
| 95 | 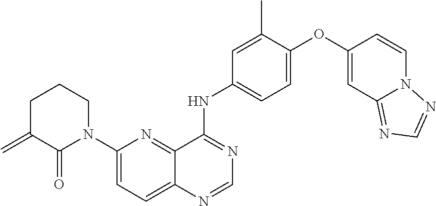 [SMILES: C=C1CCCN(c2ccc3ncnc(Nc4ccc(Oc5ccn6ncnc6c5)c(C)c4)c3n2)C1=O] | | |
| 96 | 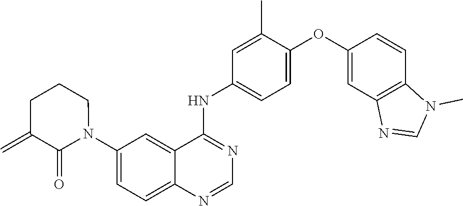 | | |
| 97 | 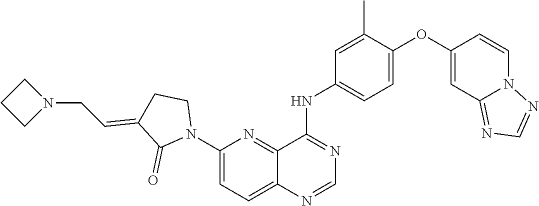 [SMILES: Cc1cc(Nc2ncnc3ccc(N4CC/C(=C\CN5CCC5)C4=O)nc23)ccc1Oc1ccn2ncnc2c1] | | |
| 98 | 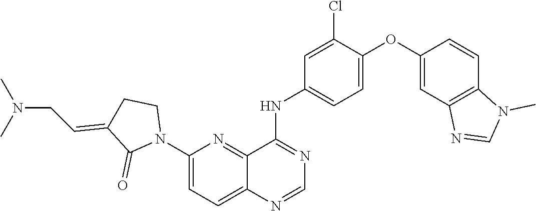 | | |
| 99 | 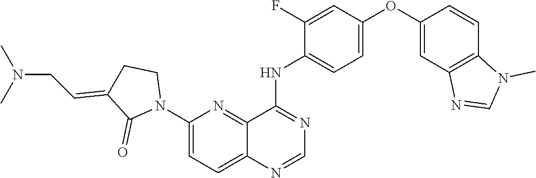 | | |
| 100 | 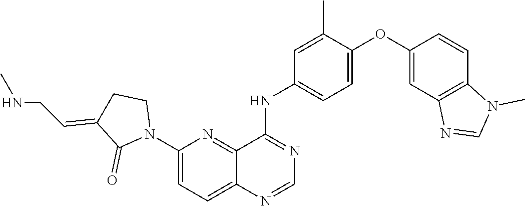 | | |
| 101 | 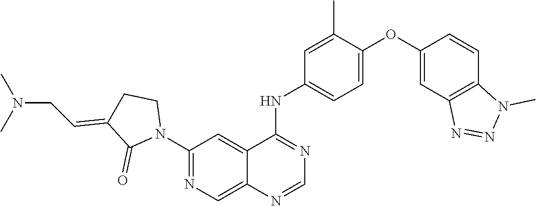 | | |
| 102 | 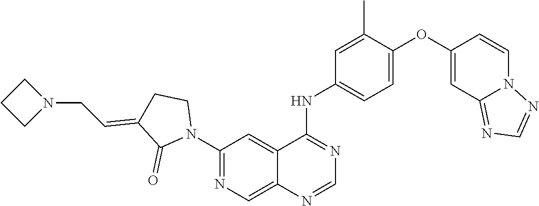 [SMILES: Cc1cc(Nc2ncnc3cnc(N4CC/C(=C\CN5CCC5)C4=O)cc23)ccc1Oc1ccn2ncnc2c1] | | |
| 103 | 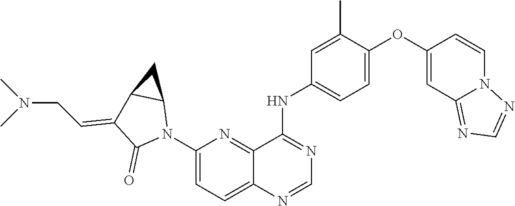 [SMILES: Cc1cc(Nc2ncnc3ccc(N4C(=O)/C(=C/CN(C)C)[C@H]5C[C@H]54)nc23)ccc1Oc1ccn2ncnc2c1] | 103-1 | 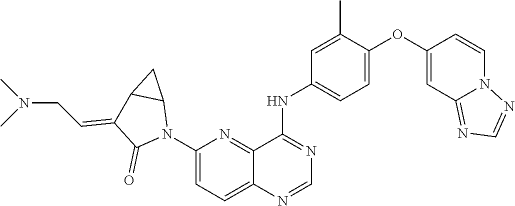 [SMILES: Cc1cc(Nc2ncnc3ccc(N4C(=O)/C(=C/CN(C)C)C5CC54)nc23)ccc1Oc1ccn2ncnc2c1] |
| 104 | 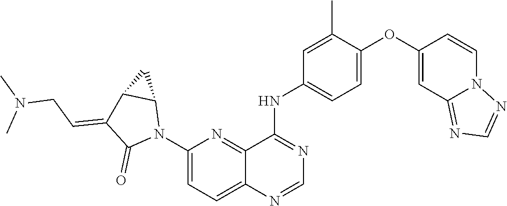 [SMILES: Cc1cc(Nc2ncnc3ccc(N4C(=O)/C(=C/CN(C)C)[C@@H]5C[C@@H]54)nc23)ccc1Oc1ccn2ncnc2c1] | 103-1 | 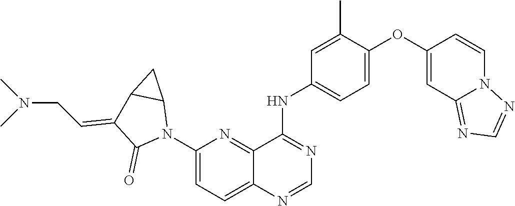 [SMILES: Cc1cc(Nc2ncnc3ccc(N4C(=O)/C(=C/CN(C)C)C5CC54)nc23)ccc1Oc1ccn2ncnc2c1] |
| 105 | 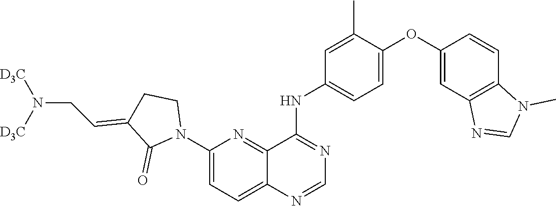 [SMILES: [2H]C([2H])([2H])N(C/C=C1\CCN(c2ccc3ncnc(Nc4ccc(Oc5ccc6c(c5)ncn6C)c(C)c4)c3n2)C1=O)C([2H])([2H])[2H]] | | |
| 106 | 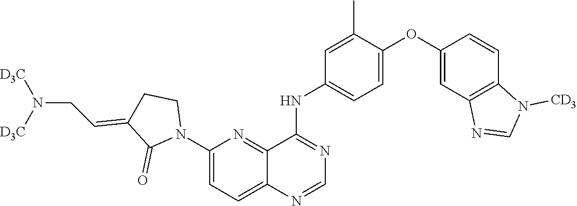 [SMILES: [2H]C([2H])([2H])N(C/C=C1\CCN(c2ccc3ncnc(Nc4ccc(Oc5ccc6c(c5)ncn6C([2H])([2H])[2H])c(C)c4)c3n2)C1=O)C([2H])([2H])[2H]] | | |
| 107 | 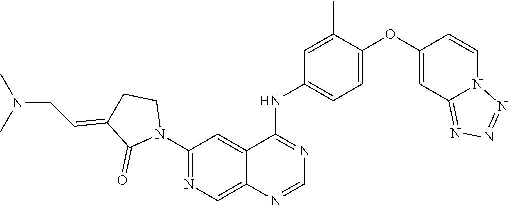 [SMILES: Cc1cc(Nc2ncnc3cnc(N4CC/C(=C\CN(C)C)C4=O)cc23)ccc1Oc1ccn2nnnc2c1] | | |
| 108 | 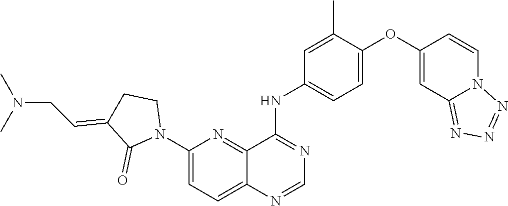 [SMILES: Cc1cc(Nc2ncnc3ccc(N4CC/C(=C\CN(C)C)C4=O)nc23)ccc1Oc1ccn2nnnc2c1] | | |
| 109 | 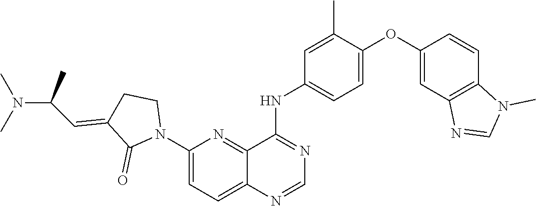 | 109-1 | 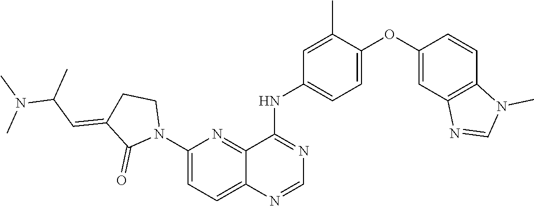 |
| 110 | 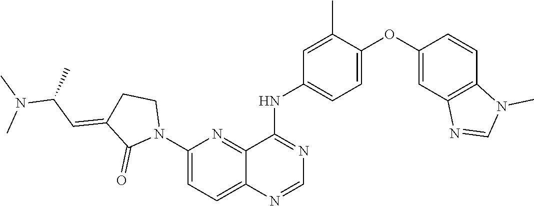 | 109-1 | 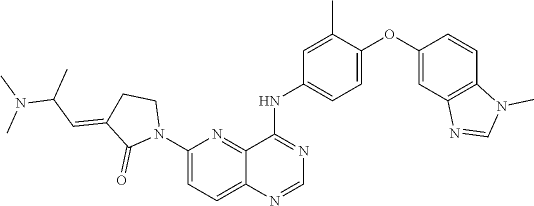 |
| 111 | 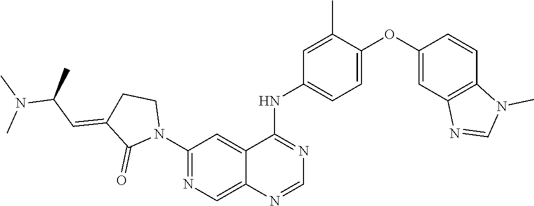 | 111-1 | 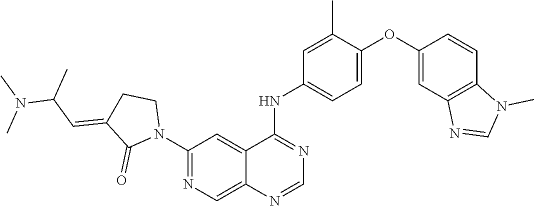 |
| 112 | 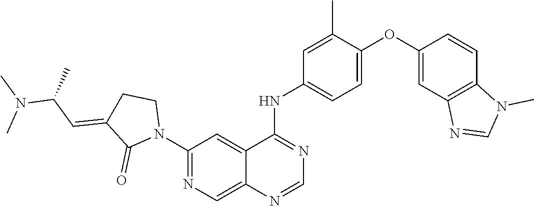 | 111-1 | 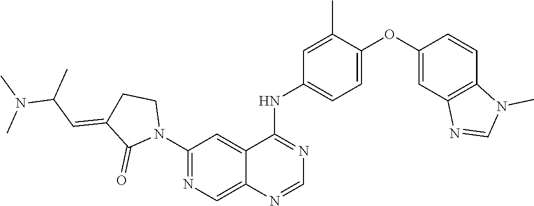 |
| 113 | 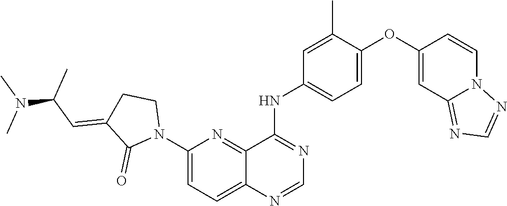 [SMILES: Cc1cc(Nc2ncnc3ccc(N4CC/C(=C\[C@H](C)N(C)C)C4=O)nc23)ccc1Oc1ccn2ncnc2c1] | 113-1 | 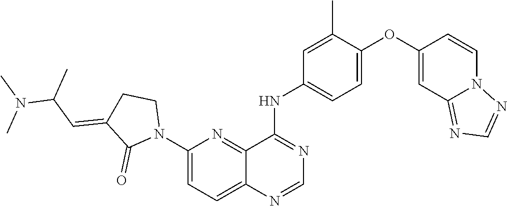 [SMILES: Cc1cc(Nc2ncnc3ccc(N4CC/C(=C\C(C)N(C)C)C4=O)nc23)ccc1Oc1ccn2ncnc2c1] |
| 114 | 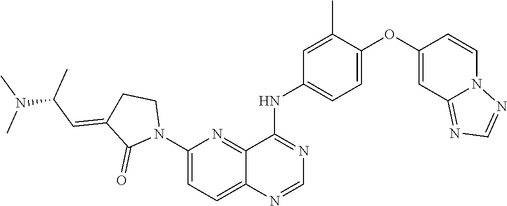 [SMILES: Cc1cc(Nc2ncnc3ccc(N4CC/C(=C\[C@@H](C)N(C)C)C4=O)nc23)ccc1Oc1ccn2ncnc2c1] | 113-1 | 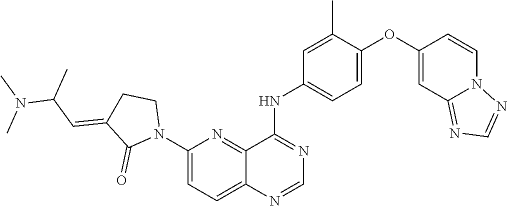 [SMILES: Cc1cc(Nc2ncnc3ccc(N4CC/C(=C\C(C)N(C)C)C4=O)nc23)ccc1Oc1ccn2ncnc2c1] |
| 115 | 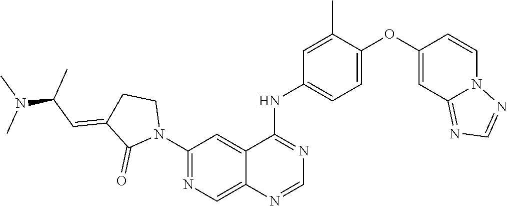 [SMILES: Cc1cc(Nc2ncnc3cnc(N4CC/C(=C\[C@H](C)N(C)C)C4=O)cc23)ccc1Oc1ccn2ncnc2c1] | 115-1 | 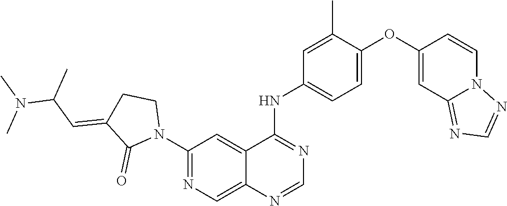 [SMILES: Cc1cc(Nc2ncnc3cnc(N4CC/C(=C\C(C)N(C)C)C4=O)cc23)ccc1Oc1ccn2ncnc2c1] |
| 116 | 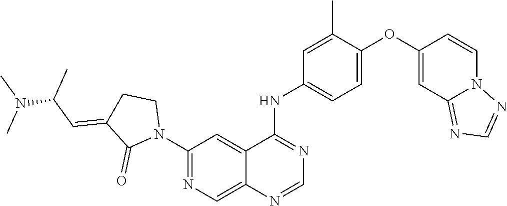 [SMILES: Cc1cc(Nc2ncnc3cnc(N4CC/C(=C\[C@@H](C)N(C)C)C4=O)cc23)ccc1Oc1ccn2ncnc2c1] | 115-1 | 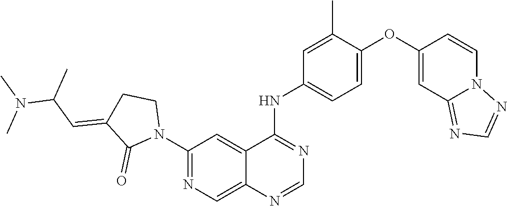 [SMILES: Cc1cc(Nc2ncnc3cnc(N4CC/C(=C\C(C)N(C)C)C4=O)cc23)ccc1Oc1ccn2ncnc2c1] |
| 117 | 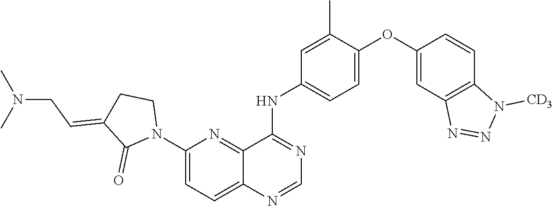 [SMILES: [2H]C([2H])([2H])n1nnc2cc(Oc3ccc(Nc4ncnc5ccc(N6CC/C(=C\CN(C)C)C6=O)nc45)cc3C)ccc21] | | |
| 118 | 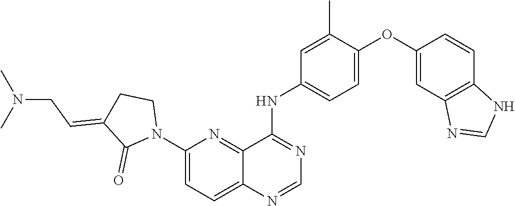 [SMILES: Cc1cc(Nc2ncnc3ccc(N4CC/C(=C\CN(C)C)C4=O)nc23)ccc1Oc1ccc2[nH]cnc2c1] | | |
| 119 | 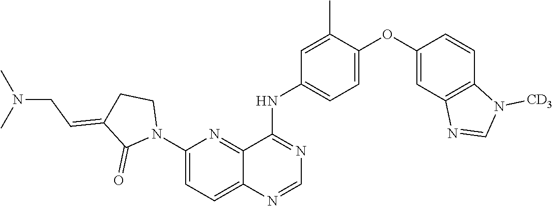 [SMILES: [2H]C([2H])([2H])n1cnc2cc(Oc3ccc(Nc4ncnc5ccc(N6CC/C(=C\CN(C)C)C6=O)nc45)cc3C)ccc21] | | |
| 120 | 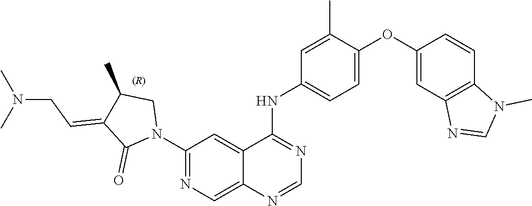 | 120-1 | 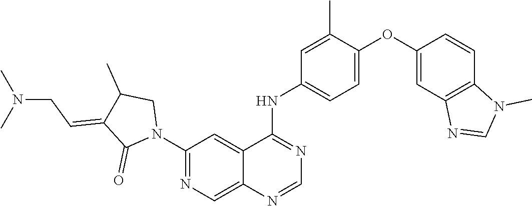 |
| 121 | 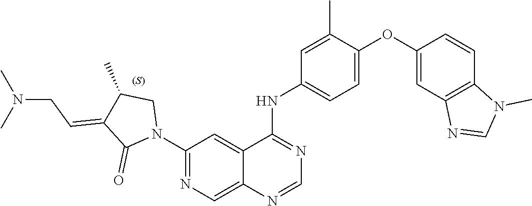 | 120-1 | 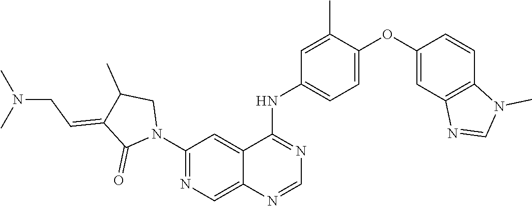 |
| 122 | 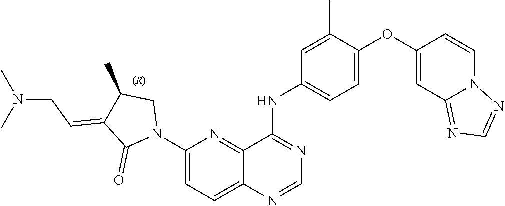 | 122-1 | 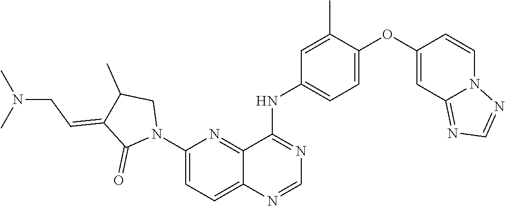 [SMILES: Cc1cc(Nc2ncnc3ccc(N4CC(C)/C(=C\CN(C)C)C4=O)nc23)ccc1Oc1ccn2ncnc2c1] |
| 123 | 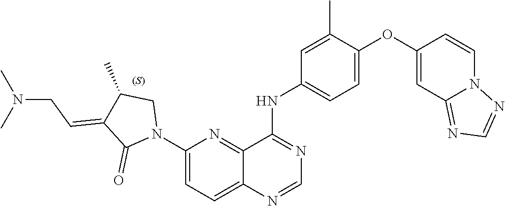 | 122-1 | 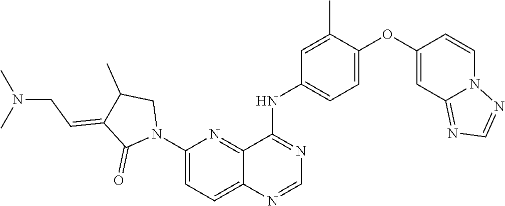 [SMILES: Cc1cc(Nc2ncnc3ccc(N4CC(C)/C(=C\CN(C)C)C4=O)nc23)ccc1Oc1ccn2ncnc2c1] |
| 124 | 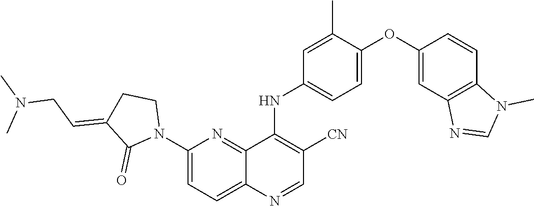 | | |
| 125 | 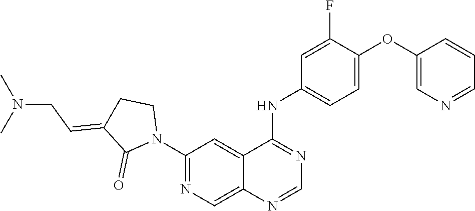 [SMILES: CN(C)C/C=C1\CCN(c2cc3c(Nc4ccc(Oc5cccnc5)c(F)c4)ncnc3cn2)C1=O] | | |
| 126 | 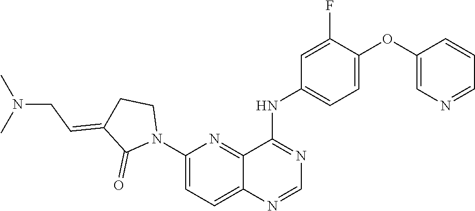 [SMILES: CN(C)C/C=C1\CCN(c2ccc3ncnc(Nc4ccc(Oc5cccnc5)c(F)c4)c3n2)C1=O] | | |
| 127 | 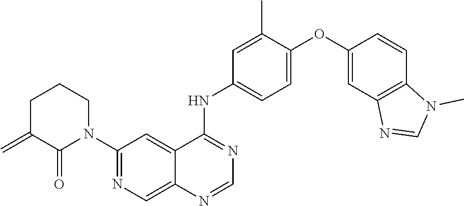 | | |
| 128 | 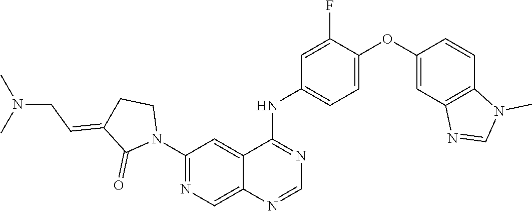 | | |
| 129 | 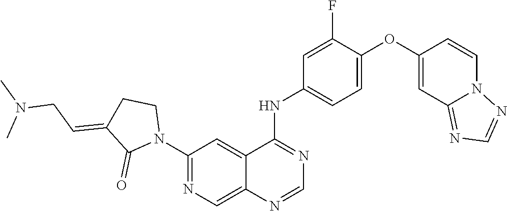 [SMILES: CN(C)C/C=C1\CCN(c2cc3c(Nc4ccc(Oc5ccn6ncnc6c5)c(F)c4)ncnc3cn2)C1=O] | | |
| 130 | 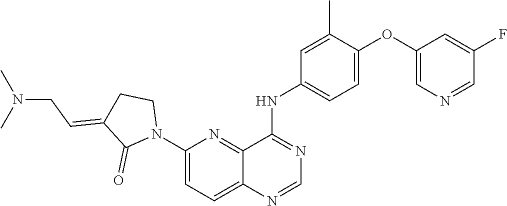 [SMILES: Cc1cc(Nc2ncnc3ccc(N4CC/C(=C\CN(C)C)C4=O)nc23)ccc1Oc1cncc(F)c1] | | |
| 131 | 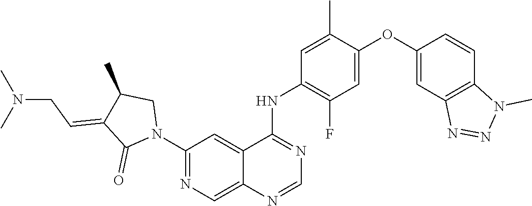 | 131- | 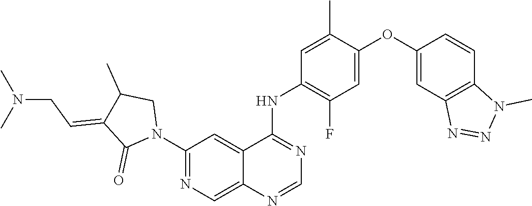 |
| 132 | 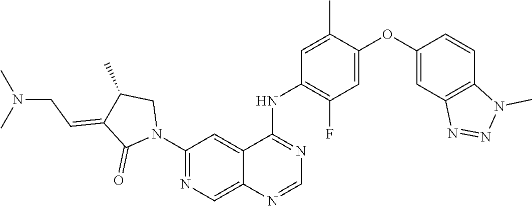 | 131-1 | 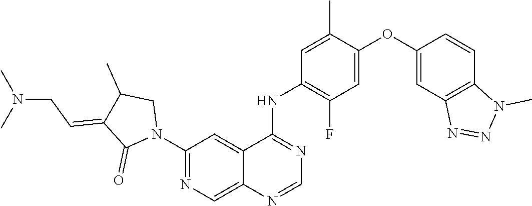 |
| 133 | 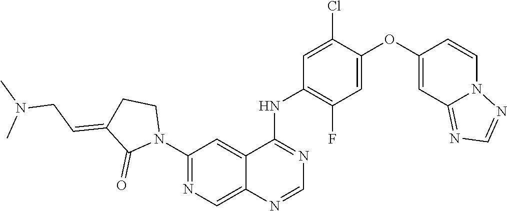 [SMILES: CN(C)C/C=C1\CCN(c2cc3c(Nc4cc(Cl)c(Oc5ccn6ncnc6c5)cc4F)ncnc3cn2)C1=O] | | |
| 134 | 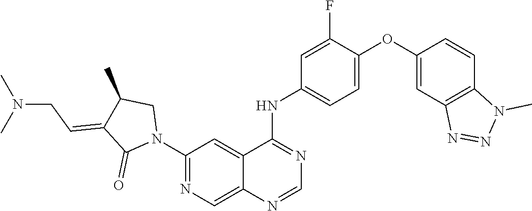 | 134-1 | 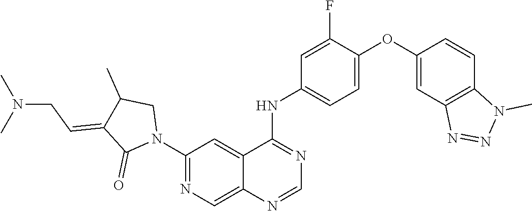 |
| 135 | 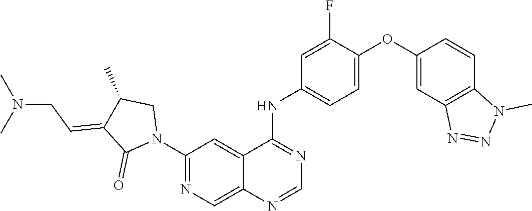 | 134-1 | 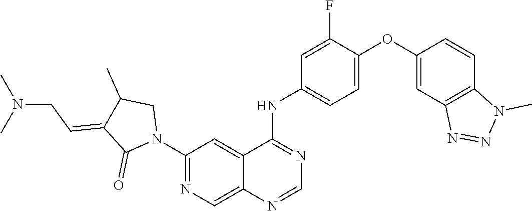 |
| 136 | 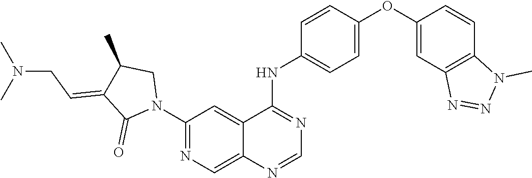 | 136-1 | 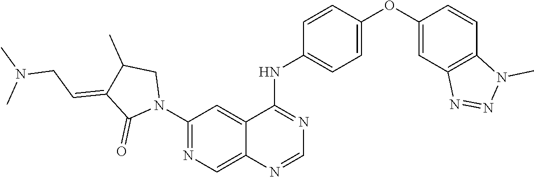 |
| 137 | 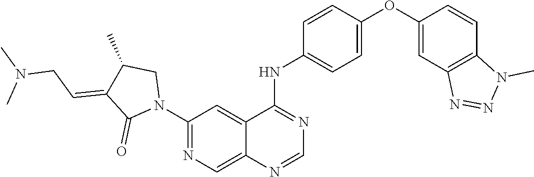 | 136-1 | 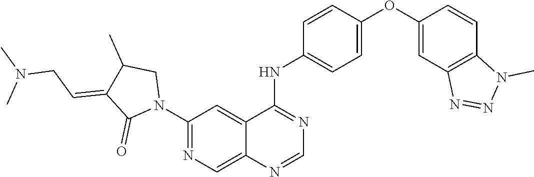 |
| 138 | 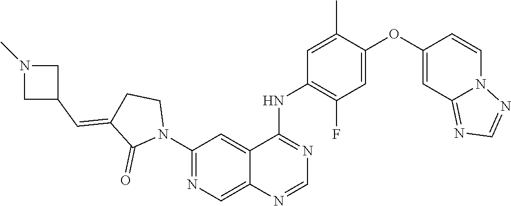 [SMILES: Cc1cc(Nc2ncnc3cnc(N4CC/C(=C\C5CN(C)C5)C4=O)cc23)c(F)cc1Oc1ccn2ncnc2c1] | | |
| 139 | 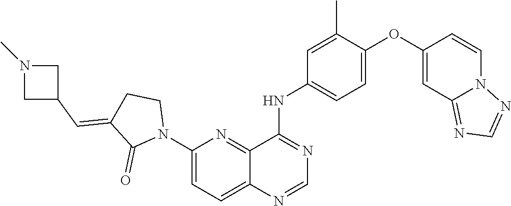 [SMILES: Cc1cc(Nc2ncnc3ccc(N4CC/C(=C\C5CN(C)C5)C4=O)nc23)ccc1Oc1ccn2ncnc2c1] | | |
| 140 | 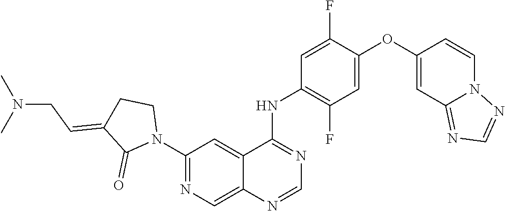 [SMILES: CN(C)C/C=C1\CCN(c2cc3c(Nc4cc(F)c(Oc5ccn6ncnc6c5)cc4F)ncnc3cn2)C1=O] | | |
| 141 | 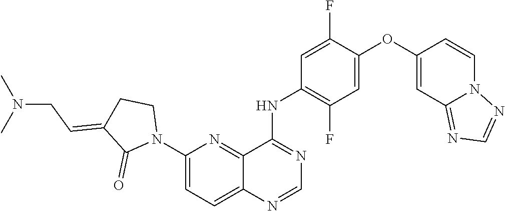 [SMILES: CN(C)C/C=C1\CCN(c2ccc3ncnc(Nc4cc(F)c(Oc5ccn6ncnc6c5)cc4F)c3n2)C1=O] | | |
| 142 | 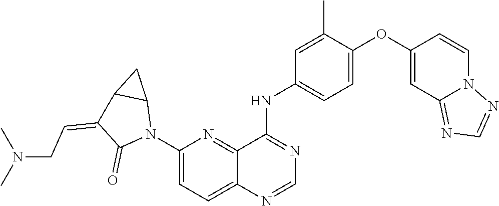 [SMILES: Cc1cc(Nc2ncnc3ccc(N4C(=O)/C(=C\CN(C)C)C5CC54)nc23)ccc1Oc1ccn2ncnc2c1] | | |
| 143 | 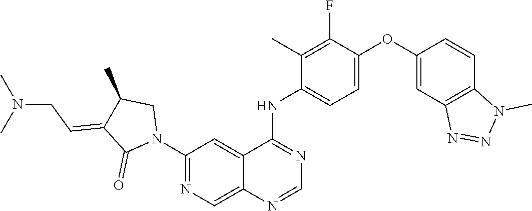 | 143-1 | 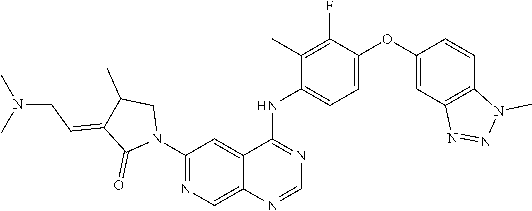 |
| 144 | 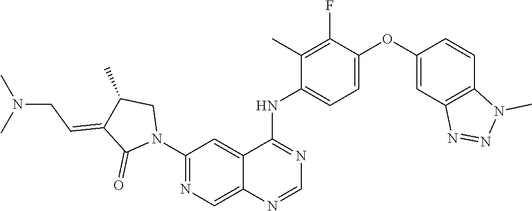 | 143-1 | 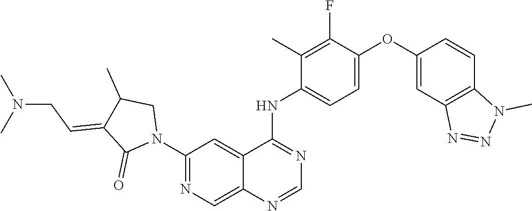 |
| 145 | 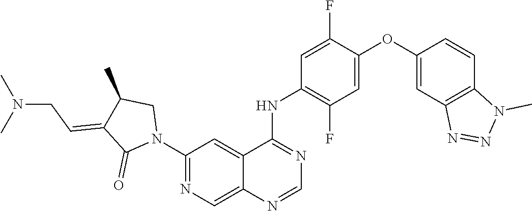 | 145-1 | 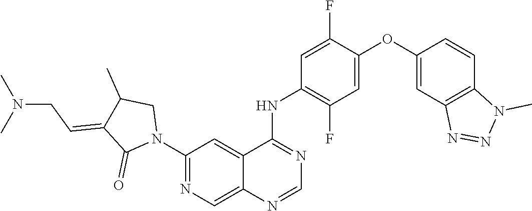 |
| 146 | 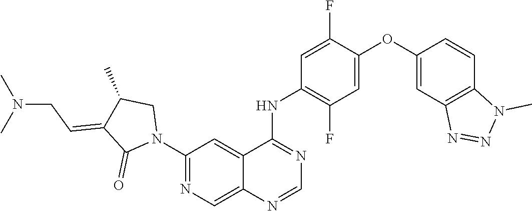 | 145-1 | 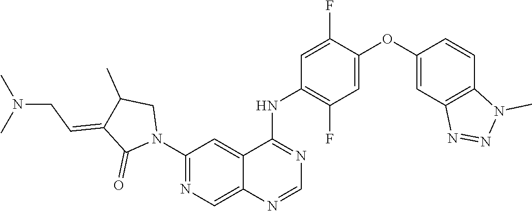 |
| 147 | 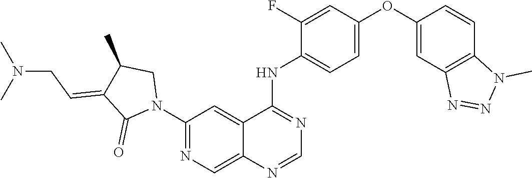 | 147-1 | 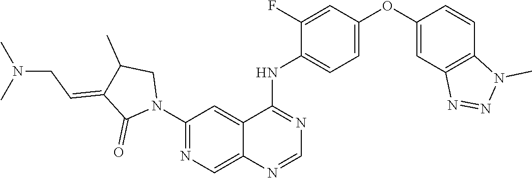 |
| 148 | 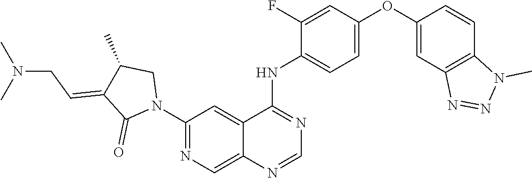 | 147-1 | 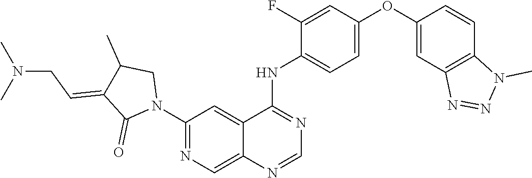 |
| 149 | 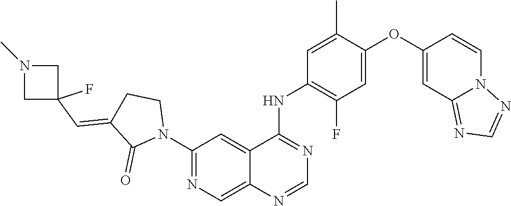 [SMILES: Cc1cc(Nc2ncnc3cnc(N4CC/C(=C\C5(F)CN(C)C5)C4=O)cc23)c(F)cc1Oc1ccn2ncnc2c1] | | |
| 150 | 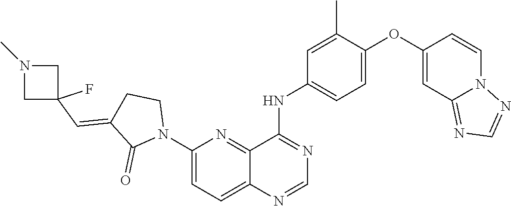 [SMILES: Cc1cc(Nc2ncnc3ccc(N4CC/C(=C\C5(F)CN(C)C5)C4=O)nc23)ccc1Oc1ccn2ncnc2c1] | | |
| 151 | 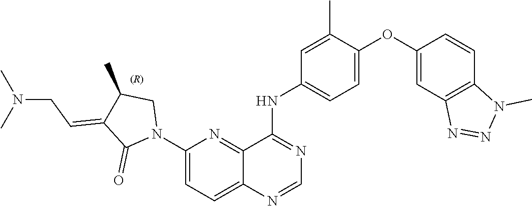 | 151-1 | 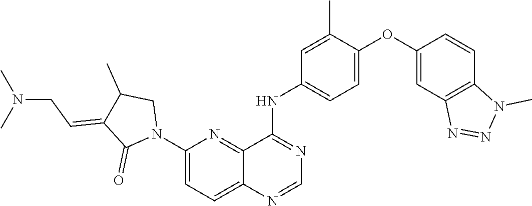 |
| 152 | 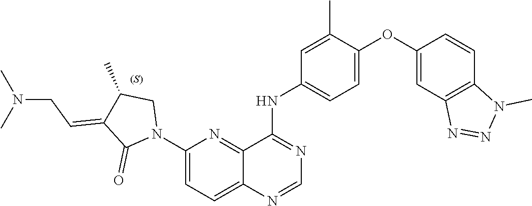 | 151-1 | 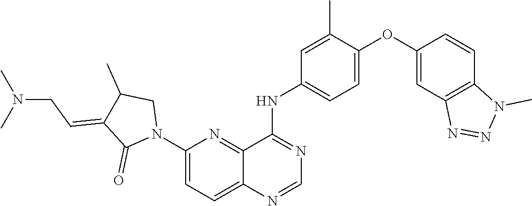 |
| 153 | 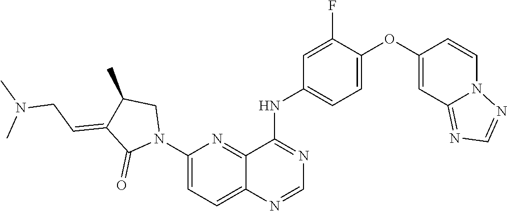 [SMILES: C[C@H]1CN(c2ccc3ncnc(Nc4ccc(Oc5ccn6ncnc6c5)c(F)c4)c3n2)C(=O)/C1=C/CN(C)C] | 153-1 | 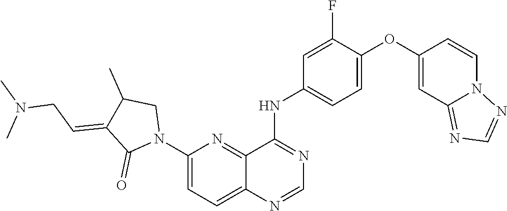 [SMILES: CC1CN(c2ccc3ncnc(Nc4ccc(Oc5ccn6ncnc6c5)c(F)c4)c3n2)C(=O)/C1=C/CN(C)C] |
| 154 | 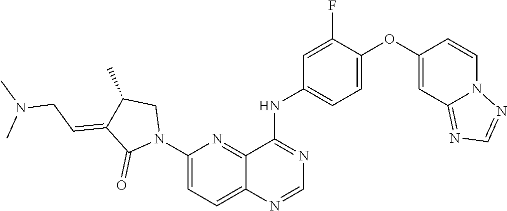 [SMILES: C[C@@H]1CN(c2ccc3ncnc(Nc4ccc(Oc5ccn6ncnc6c5)c(F)c4)c3n2)C(=O)/C1=C/CN(C)C] | 153-1 | 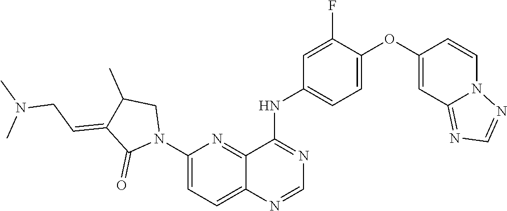 [SMILES: CC1CN(c2ccc3ncnc(Nc4ccc(Oc5ccn6ncnc6c5)c(F)c4)c3n2)C(=O)/C1=C/CN(C)C] |
| 155 | 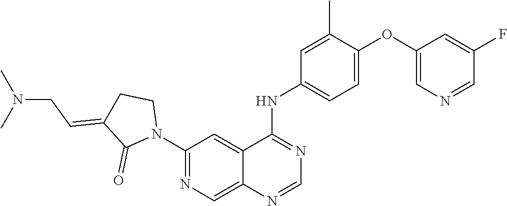 [SMILES: Cc1cc(Nc2ncnc3cnc(N4CC/C(=C\CN(C)C)C4=O)cc23)ccc1Oc1cncc(F)c1] | | |
| 156 | 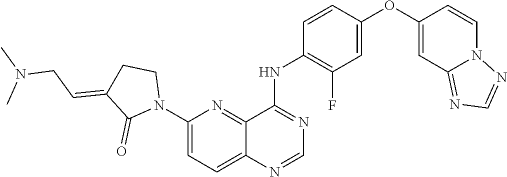 [SMILES: CN(C)C/C=C1\CCN(c2ccc3ncnc(Nc4ccc(Oc5ccn6ncnc6c5)cc4F)c3n2)C1=O] | | |

Although certain compounds described in Table 1 are presented as specific stereoisomers and/or in a non-stereochemical form, it is understood that any or all non-stereochemical forms and any or all stereochemical forms, including any enantiomeric or diastereomeric forms, and any tautomers or other forms of any of the compounds of Table 1 are herein described. In some embodiments, the compound described herein is selected from Compound Nos. 1-156.

This disclosure also includes all salts, such as pharmaceutically acceptable salts, of compounds referred to herein. This disclosure also includes any or all of the stereochemical forms, including any enantiomeric or diastereomeric forms, and any tautomers or other forms, such as N-oxides, solvates, hydrates, or isotopomers, of the compounds described. The present disclosure also includes co-crystals of the compounds described herein. Unless stereochemistry is explicitly indicated in a chemical structure or name, the structure or name is intended to embrace all possible stereoisomers of a compound depicted. In addition, where a specific stereochemical form is depicted, it is understood that other stereochemical forms are also embraced by the invention. All forms of the compounds are also embraced by the invention, such as crystalline or non-crystalline forms of the compounds. Compositions comprising a compound of the invention are also intended, such as a composition of substantially pure compound, including a specific stereochemical form thereof. Compositions comprising a mixture of compounds of the invention in any ratio are also embraced by the invention, including mixtures of two or more stereochemical forms of a compound of the invention in any ratio, such that racemic, non-racemic, enantioenriched and scalemic mixtures of a compound are embraced.

### III. Pharmaceutical Compositions and Formulations

Any of the compounds described herein may be formulated as a pharmaceutically acceptable composition.

Pharmaceutical compositions of any of the compounds detailed herein are embraced by this disclosure. Thus, the present disclosure includes pharmaceutical compositions comprising a compound as detailed herein, or a pharmaceutically acceptable salt, solvate, hydrate, or co-crystal thereof, or a mixture of any of the foregoing, and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound, or a pharmaceutically acceptable salt, solvate, hydrate, or co-crystal thereof, or a mixture of any of the foregoing, as detailed herein are provided, such as compositions of substantially pure compounds. In some embodiments, a composition containing a compound, or a pharmaceutically acceptable salt, solvate, hydrate, or co-crystal thereof, or a mixture of any of the foregoing, as detailed herein is in substantially pure form. In one variation, “substantially pure” intends a composition that contains no more than 35% impurity, wherein the impurity denotes a compound other than the compound comprising the majority of the composition or a salt thereof. For example, a composition of a substantially pure compound selected from a compound of Table 1 intends a composition that contains no more than 35% impurity, wherein the impurity denotes a compound other than the compound of Table 1. In one variation, a composition of substantially pure compound, or a pharmaceutically acceptable salt, solvate, hydrate, or co-crystal thereof, or a mixture of any of the foregoing, is provided wherein the composition contains no more than 25% impurity. In another variation, a composition of substantially pure compound, or a pharmaceutically acceptable salt, solvate, hydrate, or co-crystal thereof, or a mixture of any of the foregoing, is provided wherein the composition contains or no more than 20% impurity. In still another variation, a composition of substantially pure compound, or a pharmaceutically acceptable salt, solvate, hydrate, or co-crystal thereof, or a mixture of any of the foregoing, is provided wherein the composition contains or no more than 10% impurity. In a further variation, a composition of substantially pure compound, or a pharmaceutically acceptable salt, solvate, hydrate, or co-crystal thereof, or a mixture of any of the foregoing, is provided wherein the composition contains no more than 5% impurity. In another variation, a composition of substantially pure compound, or a pharmaceutically acceptable salt, solvate, hydrate, or co-crystal thereof, or a mixture of any of the foregoing, is provided wherein the composition contains no more than 3% impurity. In still another variation, a composition of substantially pure compound, or a pharmaceutically acceptable salt, solvate, hydrate, or co-crystal thereof, or a mixture of any of the foregoing, is provided wherein the composition contains no more than 1% impurity. In a further variation, a composition of substantially pure compound, or a pharmaceutically acceptable salt, solvate, hydrate, or co-crystal thereof, or a mixture of any of the foregoing, is provided wherein the composition contains no more than 0.5% impurity. In yet other variations, a composition of substantially pure compound means that the composition contains no more than 15%, no more than 10%, no more than 5%, no more than 3%, or no more than 1% impurity, which impurity may be the compound in a different stereochemical form. For instance, and without limitation, a composition of substantially pure (S) compound means that the composition contains no more than 15% or no more than 10% or no more than 5% or no more than 3% or no more than 1% of the (R) form of the compound.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein. In some embodiments, the compounds and compositions as provided herein are sterile. Methods for sterilization known in the art may be suitable for any compounds or form thereof and compositions thereof as detailed herein.

A compound detailed herein, or a pharmaceutically acceptable salt, solvate, hydrate, or co-crystal thereof, or a mixture of any of the foregoing, may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound, or a pharmaceutically acceptable salt, solvate, hydrate, or co-crystal thereof, or a mixture of any of the foregoing, may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

A compound detailed herein, or a pharmaceutically acceptable salt, solvate, hydrate, or co-crystal thereof, or a mixture of any of the foregoing, can be used in the preparation of a formulation, such as a pharmaceutical formulation, by combining the compound or compounds, or a pharmaceutically acceptable salt, solvate, hydrate, or co-crystal thereof, or a mixture of any of the foregoing, with a pharmaceutically acceptable carrier. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, PA, 20th ed. (2000), which is incorporated herein by reference.

A compound detailed herein, or a pharmaceutically acceptable salt, solvate, hydrate, or co-crystal thereof, or a mixture of any of the foregoing, may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid polyols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds, or a pharmaceutically acceptable salt, solvate, hydrate, or co-crystal thereof, or a mixture of any of the foregoing, described herein can be formulated in a tablet in any dosage form described, for example, a compound as described herein, or a pharmaceutically acceptable salt, solvate, hydrate, or co-crystal thereof, or a mixture of any of the foregoing, can be formulated as a 10 mg tablet.

Compositions comprising a compound, or a pharmaceutically acceptable salt, solvate, hydrate, or co-crystal thereof, or a mixture of any of the foregoing, provided herein are also described. In one variation, the composition comprises a compound, or a pharmaceutically acceptable salt, solvate, hydrate, or co-crystal thereof, or a mixture of any of the foregoing, and a pharmaceutically acceptable carrier or excipient. I n another variation, a composition of substantially pure compound, or a pharmaceutically acceptable salt, solvate, hydrate, or co-crystal thereof, or a mixture of any of the foregoing, is provided. In some embodiments, the composition is for use as a human or veterinary medicament. In some embodiments, the composition is for use in a method described herein. In some embodiments, the composition is for use in the treatment of a disease or disorder described herein.

Compositions formulated for co-administration of a compound provided herein and one or more additional pharmaceutical agents are also described. The co-administration can be simultaneous or sequential in any order. A compound provided herein may be formulated for co-administration with the one or more additional pharmaceutical agents in the same dosage form (e.g., single tablet or single i.v.) or separate dosage forms (e.g., two separate tablets, two separate i.v., or one tablet and one i.v.). Furthermore, co-administration can be, for example, 1) concurrent delivery, through the same route of delivery (e.g., tablet or i.v.), 2) sequential delivery on the same day, through the same route or different routes of delivery, or 3) delivery on different days, through the same route or different routes of delivery.

### IV. Methods of Use

Compounds and compositions detailed herein, such as a pharmaceutical composition containing a compound of formula (I), a compound of formula (II-a), a compound of formula (II-b), a compound of formula (II-c), a compound of formula (II-d), a compound of formula (III), a compound of formula (IV), or any variation thereof provided herein, or a pharmaceutically acceptable salt, solvate, hydrate, or co-crystal thereof, or a mixture of any of the foregoing, and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

In one aspect, provided herein is a method of inhibiting kinase activity of a human receptor tyrosine kinase ErbB2 or a mutant form of human ErbB2, comprising contacting the ErbB2 or the mutant form with a therapeutically effective amount of a compound or composition provided herein. In some embodiments, provided herein is a method of inhibiting kinase activity of a human receptor tyrosine kinase ErbB2 or a mutant form of human ErbB2 in a cell, comprising administering an effective amount of a compound or composition of the disclosure to the cell. In some embodiments, provided herein is a method of inhibiting kinase activity of a human receptor tyrosine kinase ErbB2 or a mutant form of human ErbB2 in an individual in need thereof, comprising administering an effective amount of a compound or composition of the disclosure to the individual.

In some embodiments, the mutant form of human ErbB2 comprises a mutation in Exon 20 that introduces certain amino acid deletions and/or insertions selected from the group consisting of: A775_A776insYVMA, G778_P780insGSP, G776delinsVC, P780_Y781insGSP, M774delinsWLV, A775_G776insSVMA, A775_G776insI, G776delinsLC, G778_S779InsCPG, V777_G778insGSP. In other embodiments, the mutant form of human ErbB2 comprises one or more mutations that introduce certain amino acid substitutions selected from the group consisting of: P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, N1219S, and A1232fs. In still further embodiments of the present aspect, the mutant form of human ErbB2 comprises one or more point mutations in ErbB2 that introduce (a) an amino acid substitution selected from the group consisting of P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, and N1219S; or (b) a frameshift at A1232.

In some variations, the compounds provided herein are selective for inhibiting human receptor tyrosine kinase ErbB2. As such, in some embodiments, provided herein is a method of selectively inhibiting human receptor tyrosine kinase ErbB2, as compared to other receptor tyrosine kinases, including but not limited to ErbB1 (EGFR), ErbB3, or ErbB4.

The compounds and compositions described herein may be used in a method of treating a disease or disorder in an individual, wherein the individual has cells or cell tissue having increased ErbB2 kinase activity, for example, as compared to the ErbB2 kinase activity in a corresponding cell type or cell tissue from a healthy individual. In some embodiments, the compound or composition is administered according to a dosage described herein.

In some embodiments, provided herein is a method for treating a disease or disorder in an individual, wherein the individual has cells or cell tissue having increased ErbB2 kinase activity, comprising administering to an individual in need of treatment a therapeutically effective amount of a compound of formula (I), a compound of formula (II-a), a compound of formula (II-b), a compound of formula (II-c), a compound of formula (II-d), a compound of formula (III), a compound of formula (IV), or any variation thereof, or a pharmaceutically acceptable salt, solvate, hydrate, or co-crystal thereof, or a mixture of any of the foregoing, or a therapeutically effective amount of a composition as described herein. In some embodiments, the disease or disorder is cancer. In some embodiments, the disease or disorder is lung cancer, glioma, head and/or neck cancer, salivary gland cancer, breast cancer, esophageal cancer, liver cancer, stomach (gastric) cancer, uterine cancer, cervical cancer, biliary tract cancer, pancreatic cancer, colorectal cancer, renal cancer, bladder cancer, or prostate cancer. In some embodiments, the cancer is non-small cell lung cancer. In some embodiments, the individual has received at least one, at least two or at least three prior therapies for the cancer. In certain embodiments, the one or more prior therapies are selected from the group consisting of lapatinib, neratinib, afatinib, pyrotinib, poziotinib, TAK-788 and tucatinib.

In some embodiments, the disease or disorder is refractory or resistant to first-line treatment, second-line treatment, and/or third-line treatment. In certain embodiments, the condition having increased activation of ErbB2 kinase activity is refractory or resistant to treatment with one or more tyrosine kinase inhibitors selected from the group consisting of lapatinib, neratinib, afatinib, pyrotinib, poziotinib, TAK-788, and tucatinib.

Resistant subtypes of tyrosine kinase-mediated diseases or disorders may be associated with any number of ErbB2 independent resistance mechanisms. In some embodiments wherein the disease or disorder in the individual having cells or cell tissue with increased ErbB2 kinase activity is refractory to treatment, the disease or disorder is characterized as being associated with one or more ErbB2 dependent resistance mechanisms. ErbB2-dependent resistance mechanisms include, but are not limited to, one or more mutations in Exon 20 of ErbB2 or other disease-associated point mutations. The one or more mutations of ErbB2 introduce certain amino acid deletions and/or insertions, for example, A775_A776insYVMA, G778_P780insGSP, G776delinsVC, P780_Y781insGSP, M774delinsWLV, A775_G776insSVMA, A775_G776insI, G776delinsLC, G778_S779InsCPG, and/or V777 G778insGSP. In other variations, the mutations introduce certain amino acid substitutions, for example, P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, N1219S, and/or A1232fs. In some variations, the mutations introduce certain (a) amino acid substitutions, for example, P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, and N1219S, and/or (b) frameshifts, such as a frameshift at A1232. In some variations, the refractory disease or disorder in an individual having increased activation of the ErbB2 kinase activity is associated with one or more mutations in Exon 20 of the ErbB2. In certain variations, the one or more mutations in Exon 20 of the ErbB2 that introduce certain amino acid deletions and/or insertions selected from the group consisting of: A775_A776insYVMA, G778_P780insGSP, G776delinsVC, P780_Y781insGSP, M774delinsWLV, A775_G776insSVMA, A775_G776insI, G776delinsLC, G778_S779InsCPG, and V777_G778insGSP. In other variations, the refractory disease or disorder in an individual having increased activation of the ErbB2 kinase activity is associated with one or more disease-associated point mutations. In certain variations, the one or more point mutations introduce certain amino acid substitutions selected from the group consisting of: P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, N1219S, and A1232fs. In other embodiments, the one or more point mutations introduce (a) an amino acid substitution selected from the group consisting of P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, and N1219S; or (b) a frameshift at A1232.

In some embodiments, provided is a method for treating cancer in an individual in need thereof, comprising administering to the individual a therapeutically effective amount of a compound of formula (I), a compound of formula (II-a), a compound of formula (II-b), a compound of formula (II-c), a compound of formula (II-d), a compound of formula (III), a compound of formula (IV), or any variation thereof as described herein, or a therapeutically effective amount of a composition as described herein. In some embodiments, the cancer comprises cells or cell tissue having increased ErbB2 kinase activity, for example, as compared to the ErbB2 kinase activity in a corresponding cell type or cell tissue from a healthy individual.

In some embodiments, the cancer comprises cells or cell tissue having one or more mutations in Exon 20 of the ErbB2. In certain embodiments, the one or more mutations in Exon 20 of the ErbB2 introduce certain amino acid deletions and/or insertions selected from the group consisting of A775_A776insYVMA, G778_P780insGSP, G776delinsVC, P780_Y781insGSP, M774delinsWLV, A775_G776insSVMA, A775_G776insI, G776delinsLC, G778_S779InsCPG, and V777_G778insGSP. In some embodiments, the cancer comprises cells or cell tissue comprising one or more disease-associated point mutations. In certain embodiments, the one or more point mutations introduce certain amino acid substitutions selected from the group consisting of: P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, N1219S, and A1232fs. In certain other embodiments, the cancer comprises cells or cell tissue having one or more point mutations that introduce (a) an amino acid substitution selected from the group consisting of P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, and N1219S; or (b) a frameshift at A1232. In some embodiments, the disease or disorder is lung cancer, glioma, head and/or neck cancer, salivary gland cancer, breast cancer, esophageal cancer, liver cancer, stomach (gastric) cancer, uterine cancer, cervical cancer, biliary tract cancer, pancreatic cancer, colorectal cancer, renal cancer, bladder cancer, or prostate cancer. In some embodiments, the cancer is non-small cell lung cancer.

In one aspect, provided herein is a method of treating cancer in an individual in need thereof, wherein modulation of ErbB2 kinase activity inhibits or ameliorates the pathology and/or symptomology of the cancer, comprising administering to the individual a therapeutically effective amount of a compound or composition provided herein. In one embodiment, provided herein is a method of treating cancer, wherein modulation of ErbB2 kinase activity inhibits the pathology and/or symptomology of the cancer, in an individual, comprising administering to the individual a therapeutically effective amount of a compound or composition provided herein. In one embodiment, provided herein is a method of treating a cancer, wherein modulation of ErbB2 kinase activity ameliorates the pathology and/or symptomology of the cancer, in an individual, comprising administering to the individual a therapeutically effective amount of a compound or composition provided herein.

In another aspect, provided herein is a method of preventing cancer, wherein modulation of ErbB2 kinase activity prevents the pathology and/or symptomology of the cancer, in an individual, comprising administering to the individual a therapeutically effective amount of a compound or composition provided herein. In another aspect, provided herein is a method of delaying the onset and/or development of a cancer in an individual (such as a human) who is at risk for developing the cancer, e.g., an individual who has cells or cell tissue having increased ErbB2 kinase activity. It is appreciated that delayed development may encompass prevention in the event the individual does not develop the cancer.

In one aspect, provided herein is a method of delaying the onset and/or development of cancer in an individual having cells or cell tissue having increased ErbB2 kinase activity in need thereof, comprising administering to the individual a therapeutically effective amount of a compound or composition provided herein. In some embodiments, the cancer is lung cancer, glioma, head and/or neck cancer, salivary gland cancer, breast cancer, esophageal cancer, liver cancer, stomach (gastric) cancer, uterine cancer, cervical cancer, biliary tract cancer, pancreatic cancer, colorectal cancer, renal cancer, bladder cancer, or prostate cancer. In some embodiments, the cancer is non-small cell lung cancer.

In one aspect, provided herein is a compound of formula (I), a compound of formula (II-a), a compound of formula (II-b), a compound of formula (II-c), a compound of formula (II-d), a compound of formula (III), a compound of formula (IV), or any variation thereof, or a pharmaceutically acceptable salt, solvate, hydrate, or co-crystal thereof, or a mixture of any of the foregoing, for use in therapy. In some embodiments, provided herein is a compound of formula (I), a compound of formula (II-a), a compound of formula (II-b), a compound of formula (II-c), a compound of formula (II-d), a compound of formula (III), a compound of formula (IV), or any variation thereof, or a pharmaceutically acceptable salt, solvate, hydrate, or co-crystal thereof, or a mixture of any of the foregoing, or pharmaceutical composition comprising such compound, for use in the treatment of cancer. In some embodiments, provided is a compound of formula (I), a compound of formula (II-a), a compound of formula (II-b), a compound of formula (II-c), a compound of formula (II-d), a compound of formula (III), a compound of formula (IV), or any variation thereof, or a pharmaceutically acceptable salt, solvate, hydrate, or co-crystal thereof, or a mixture of any of the foregoing, or a pharmaceutical composition comprising such compound, for use in the treatment of cancer. In some embodiments, provided is a compound of formula (I), a compound of formula (II-a), a compound of formula (II-b), a compound of formula (II-c), a compound of formula (II-d), a compound of formula (III), a compound of formula (IV), or any variation thereof, or a pharmaceutically acceptable salt, solvate, hydrate, or co-crystal thereof, or a mixture of any of the foregoing, or a pharmaceutical composition comprising such compound, for use in the treatment of cancer, wherein the cancer comprises cells or cell tissue having increased activation of ErbB2 kinase activity. In some embodiments, provided is a compound of formula (I), a compound of formula (II-a), a compound of formula (II-b), a compound of formula (II-c), a compound of formula (II-d), a compound of formula (III), a compound of formula (IV), or any variation thereof, or a pharmaceutically acceptable salt, solvate, hydrate, or co-crystal thereof, or a mixture of any of the foregoing, or a pharmaceutical composition comprising such compound, for use in the treatment of cancer, wherein the cancer comprises cells or cell tissue having one or more mutations in Exon 20 of the ErbB2. In some embodiments, provided is a compound of formula (I), a compound of formula (II-a), a compound of formula (II-b), a compound of formula (II-c), a compound of formula (II-d), a compound of formula (III), a compound of formula (IV), or any variation thereof, or a pharmaceutically acceptable salt, solvate, hydrate, or co-crystal thereof, or a mixture of any of the foregoing, or a pharmaceutical composition comprising such compound, for use in the treatment of cancer, wherein the cancer cells comprise one or more genetic alterations in Exon 20 of the ErbB2 that introduce certain amino acid deletions and/or insertions selected from the group consisting of A775_A776insYVMA, G778_P780insGSP, G776delinsVC, P780_Y781insGSP, M774delinsWLV, A775_G776insSVMA, A775_G776insI, G776delinsLC, G778_S779InsCPG, and V777_G778insGSP. In some embodiments, provided is a compound of formula (I), a compound of formula (II-a), a compound of formula (II-b), a compound of formula (II-c), a compound of formula (II-d), a compound of formula (III), a compound of formula (IV), or any variation thereof, or a pharmaceutically acceptable salt, solvate, hydrate, or co-crystal thereof, or a mixture of any of the foregoing, or a pharmaceutical composition comprising such compound, for use in the treatment of cancer, wherein the cancer comprises cells or cell tissue having one or more disease-associated point mutations in ErbB2. In certain embodiments, the cancer comprises cells or cell tissue having one or more point mutations that introduce certain amino acid substitutions selected from the group consisting of P122L, R217C, 1263T, A293T, S305C, S310F/Y, H470Q, 1655V, V659E, G660D, R678Q/C, L755R/S/P, 1767M, D769H/N/Y, V777L/M, V842I, R868W, H878Y, E930K/D, E1021Q, F1030C, V1128I, N1219S, and A1232fs. In certain other embodiments, the cancer comprises cells or cell tissue having one or more point mutations that introduce (a) an amino acid substitution selected from the group consisting of P122L, R217C, 1263T, A293T, S305C, S310F/Y, H470Q, 1655V, V659E, G660D, R678Q/C, L755R/S/P, 1767M, D769H/N/Y, V777L/M, V842I, R868W, H878Y, E930K/D, E1021Q, F1030C, V1128I, and N1219S; or (b) a frameshift at A1232. In some embodiments, provided is a compound of formula (I), a compound of formula (II-a), a compound of formula (II-b), a compound of formula (II-c), a compound of formula (II-d), a compound of formula (III), a compound of formula (IV), or any variation thereof, or a pharmaceutically acceptable salt, solvate, hydrate, or co-crystal thereof, or a mixture of any of the foregoing, or a pharmaceutical composition comprising such compound, for use in the treatment of lung cancer, glioma, head and/or neck cancer, salivary gland cancer, breast cancer, esophageal cancer, liver cancer, stomach (gastric) cancer, uterine cancer, cervical cancer, biliary tract cancer, pancreatic cancer, colorectal cancer, renal cancer, bladder cancer, or prostate cancer. In some embodiments, the lung cancer is non-small cell lung cancer.

In another embodiment, provided herein is a compound of formula (I), a compound of formula (II-a), a compound of formula (II-b), a compound of formula (II-c), a compound of formula (II-d), a compound of formula (III), a compound of formula (IV), or any variation thereof, or a pharmaceutically acceptable salt, solvate, hydrate, or co-crystal thereof, or a mixture of any of the foregoing, for use in the manufacture of a medicament for the treatment of cancer. In another embodiment, provided herein is a compound of formula (I), a compound of formula (II-a), a compound of formula (II-b), a compound of formula (II-c), a compound of formula (II-d), a compound of formula (III), a compound of formula (IV), or any variation thereof, or a pharmaceutically acceptable salt, solvate, hydrate, or co-crystal thereof, or a mixture of any of the foregoing, for use in the manufacture of a medicament for the treatment of cancer, wherein the cancer comprises cells or cell tissue having increased ErbB2 kinase activity. In another embodiment, provided herein is a compound of formula (I), a compound of formula (II-a), a compound of formula (II-b), a compound of formula (II-c), a compound of formula (II-d), a compound of formula (III), a compound of formula (IV), or any variation thereof, or a pharmaceutically acceptable salt, solvate, hydrate, or co-crystal thereof, or a mixture of any of the foregoing, for use in the manufacture of a medicament for the treatment of cancer, wherein the cancer cells or cancer cell tissue comprise one or more mutations in Exon 20 of the ErbB2.

In some embodiments, the medicament is for the treatment of cancer, wherein the cancer cells comprise one or more genetic alterations in Exon 20 of the ErbB2 that introduce certain amino acid deletions and/or insertions selected from the group consisting of A775_A776insYVMA, G778_P780insGSP, G776delinsVC, P780_Y781insGSP, M774delinsWLV, A775_G776insSVMA, A775_G776insI, G776delinsLC, G778_S779InsCPG, and V777_G778insGSP. In another embodiment, provided herein is a compound of formula (I), a compound of formula (II-a), a compound of formula (II-b), a compound of formula (II-c), a compound of formula (II-d), a compound of formula (III), a compound of formula (IV), or any variation thereof, or a pharmaceutically acceptable salt, solvate, hydrate, or co-crystal thereof, or a mixture of any of the foregoing, for use in the manufacture of a medicament for the treatment of cancer, wherein the cancer cells or cancer cell tissue comprise one or more disease-associated point mutations in ErbB2. In some embodiments, the medicament is for the treatment of cancer, wherein the cancer cells comprise one or more point mutations that introduce certain amino acid substitutions selected from the group consisting of: P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, N1219S, and A1232fs. In some embodiments, the medicament is for the treatment of cancer, wherein the cancer cells comprise one or more point mutations that introduce (a) an amino acid substitution selected from the group consisting of: P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, and N1219S, or (b) a frameshift at A1232. In some embodiments, the medicament is for the treatment of lung cancer, glioma, head and/or neck cancer, salivary gland cancer, breast cancer, esophageal cancer, liver cancer, stomach (gastric) cancer, uterine cancer, cervical cancer, biliary tract cancer, pancreatic cancer, colorectal cancer, renal cancer, bladder cancer, or prostate cancer. In some embodiments, the medicament is for the treatment of non-small cell lung cancer.

In some embodiments, the individual is a mammal. In some embodiments, the individual is a primate, dog, cat, rabbit, or rodent. In some embodiments, the individual is a primate. In some embodiments, the individual is a human. In some embodiments, the human is at least about or is about any of 18, 21, 30, 50, 60, 65, 70, 75, 80, or 85 years old. In some embodiments, the human is a child. In some embodiments, the human is less than about or about any of 21, 18, 15, 10, 5, 4, 3, 2, or 1 years old.

In some embodiments, the method further comprises administering one or more additional pharmaceutical agents. In some embodiments, the method further comprises administering one or more additional anti-cancer agents to the patient. In some embodiments, the method further comprises administering radiation. In some embodiments, the method further comprises administering one or more additional pharmaceutical agents and radiation.

### V. Dosing and Method of Administration

The dose of a compound described herein, or a pharmaceutically acceptable salt, solvate, hydrate, or co-crystal thereof, or a mixture of any of the foregoing, administered to an individual (such as a human) may vary with the particular compound or salt thereof, the method of administration, and the particular cancer, such as type and stage of cancer, being treated. In some embodiments, the amount of the compound, or a pharmaceutically acceptable salt, solvate, hydrate, or co-crystal thereof, or a mixture of any of the foregoing, is a therapeutically effective amount.

The compounds provided herein, or a pharmaceutically acceptable salt, solvate, hydrate, or co-crystal thereof, or a mixture of any of the foregoing, may be administered to an individual via various routes, including, e.g., intravenous, intramuscular, subcutaneous, oral, and transdermal.

The effective amount of the compound may in one aspect be a dose of between about 0.01 and about 100 mg/kg. Effective amounts or doses of the compounds of the present disclosure may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein, or a pharmaceutically acceptable salt, solvate, hydrate, or co-crystal thereof, or a mixture of any of the foregoing, and a pharmaceutically acceptable excipient.

A compound or composition provided herein may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

### VI. Articles of Manufacture and Kits

The present disclosure further provides articles of manufacture comprising a compound described herein, or a pharmaceutically acceptable salt, solvate, hydrate, or co-crystal thereof, or a mixture of any of the foregoing, a composition described herein, or one or more unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises one or more compounds described herein or a composition comprising a compound described herein. The kits may employ any of the compounds disclosed herein. In one variation, the kit employs a compound described herein, or a pharmaceutically acceptable salt, solvate, hydrate, or co-crystal thereof, or a mixture of any of the foregoing, thereof. The kits may be used for any one or more of the uses described herein, and, accordingly, may contain instructions for the treatment of any disease or described herein, for example for the treatment of cancer, including lung, glioma, skin, head and neck, salivary gland, breast, esophageal, liver, stomach (gastric), uterine, cervical, biliary tract, pancreatic, colorectal, renal, bladder or prostate cancer. In some embodiments, the kit may contain instructions for the treatment of non-small cell lung cancer.

In certain embodiments of the foregoing, the cancer comprises cells or cell tissue having one or more mutations in Exon 20 of the ErbB2. In still further embodiments, the cancer cells or cancer cell tissue comprise one or more mutations in Exon 20 of the ErbB2 that introduce certain amino acid deletions and/or insertions selected from the group consisting of A775_A776insYVMA, G778_P780insGSP, G776delinsVC, P780_Y781insGSP, M774delinsWLV, A775_G776insSVMA, A775_G776insI, G776delinsLC, G778_S779InsCPG, and V777_G778insGSP. In certain other embodiments of the cancer comprises cells or cell tissue having one or more disease-associated point mutations in ErbB2. In still further embodiments, the cancer cells or cancer cell tissue comprise the one or more point mutations that introduce amino acid substitutions selected from the group consisting of P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, N1219S, and A1232fs. In other embodiments, the cancer comprises cells or cell tissue having one or more point mutations that introduce (a) an amino acid substitution selected from the group consisting of P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, and N1219S; or (b) a frameshift at A1232.

The kits optionally further comprise a container comprising one or more additional pharmaceutical agents and which kits further comprise instructions on or in the package insert for treating the subject with an effective amount of the one or more additional pharmaceutical agents.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### VII. General Synthetic Methods

The compounds of the present disclosure may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

The intermediates described in the following preparations may contain a number of nitrogen, hydroxy, and acid protecting groups such as esters. The variable protecting group may be the same or different in each occurrence depending on the particular reaction conditions and the particular transformations to be performed. The protection and deprotection conditions are well known to the skilled artisan and are described in the literature. See. e.g., Greene and Wuts, Protective Groups in Organic Synthesis, (T. Greene and P. Wuts, eds., 2d ed. 1991).

Certain stereochemical centers have been left unspecified and certain substituents have been eliminated in the following schemes for the sake of clarity and are not intended to limit the teaching of the schemes in any way. Furthermore, individual isomers, enantiomers, and diastereomers may be separated or resolved by one of ordinary skill in the art at any convenient point in the synthesis of compounds of the invention, by methods such as selective crystallization techniques or chiral chromatography (See for example, J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994).

The compounds of the present invention, or salts thereof, may be prepared by a variety of procedures known in the art, some of which are illustrated in the Examples below.

The specific synthetic steps for each of the routes described may be combined in different ways, to prepare compounds of the invention, or salts thereof. The products of each step can be recovered by conventional methods well known in the art, including extraction, evaporation, precipitation, chromatography, filtration, trituration, and crystallization. The reagents and starting materials are readily available to one of ordinary skill in the art. Others may be made by standard techniques of organic and heterocyclic chemistry which are analogous to the syntheses of known structurally-similar compounds and the procedures described in the Examples which follow including any novel procedures.

Compounds of formula (I), formula (II-a), formula (II-b), formula (II-c), formula (II-d), formula (III), formula (IV), or Table 1 can be prepared according to Scheme A, Scheme B, Scheme C, Scheme D, Scheme E, Scheme F, Scheme G, Scheme H, Scheme I, Scheme J, Scheme K, Scheme L, Scheme M, Scheme N, Scheme O, Scheme P, Scheme Q, Scheme R, Scheme S, or Scheme T, wherein the Ring A moiety, R¹, R², R³, R⁴, R⁵, R⁶, R⁷, R⁸, n G, R^{g1}, R^{g2}, V, W, X, Y, R^{y}, and Z are as defined for formula (I), for formula (II-a), for formula (II-b), for formula (II-c), for formula (II-d), formula (III), for formula (IV), or any applicable variation thereof as detailed herein.

Compounds of formula (I), (II-a), (II-b), (II-c), (II-d), (III), (IV), (I-a), (I-b), (I-c), (I-d), (I-e), (I-f), (I-g), (I-h), (I-i), (I-a-1), (I-a-2), (I-b-1), (I-b-2), (I-c-1), (I-c-2), (I-d-1), (I-d-2), (I-e-1), (I-e-2), (I-f-1), (I-f-2), (I-g-1), (I-g-2), (I-h-1), (I-h-2), (I-i-1), (I-i-2), (I-j-1), (I-j-2), (I-j-3), (T-j-4), (T-j-5), (T-j-6), (I-j-7), (I-j-8), (T-j-9), (I j-10), (I-j-11), (I-j-12), (I-j-13), (I-j-14), (I-j-15), (I-j-16), (I-j-17), (I-j-18), (I-k-1), (I-k-2), (I-k-3), (I-k-4), (I-k-5), (I-k-6), (I-k-7), (I-k-8), (I-k-9), (I-k-10), (I-k-11), (I-k-12), (I-k-13), (I-k-14), (I-k-15), (I-k-16), (I-k-17), (I-k-18), (I-l-1), (I-l-2), (I-l-3), (I-l-4), (I-l-5), (I-l-6), (I-l-7), (I-l-8), (I-l-9), (I-l-10), (I-l-11), (I-l-12), (I-l-13), (I-l-14), (I-l-15), (I-l-16), (I-l-17), or (I-l-18), can be prepared according to Scheme A, Scheme B, Scheme C, Scheme D, Scheme E, Scheme F, Scheme G, Scheme H, Scheme I, Scheme J, Scheme K, Scheme L, Scheme M, Scheme N, Scheme O, Scheme P, Scheme Q, Scheme R, Scheme S, or Scheme T, wherein the Ring A moiety, R¹, R², R³, R⁴, R⁵, R⁶, R⁷, R⁸, R⁹, R¹⁰, m, n, p, G, R^{g1}, R^{g2}, V, W X, Y, R^{y}, and Z are as defined for formula (I), formula (II-a), formula (II-b), formula (II-c), formula (II-d), for formula (III), for formula (IV), or any applicable variation thereof as detailed herein.

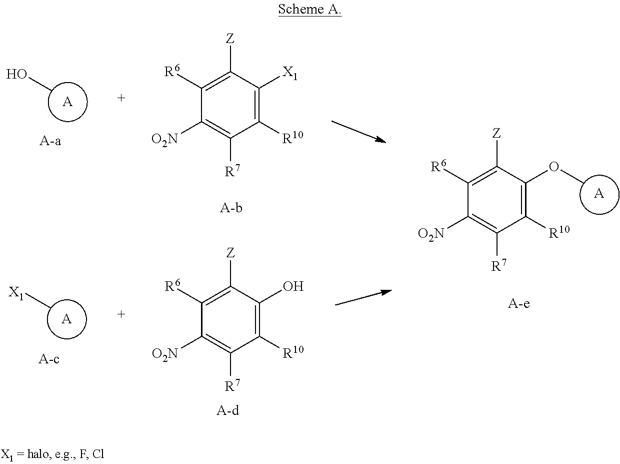

As shown in Scheme A, nucleophilic substitution by hydroxylated Ring A-containing heterocycles of general formula A-a of nitrosylated benzenes of general formula A-b provides the coupled ether compounds of general formula A-e. Alternatively, compounds of general formula A-c may be reacted with compounds of general formula A-d to yield the coupled ether compounds of general formula A-e.

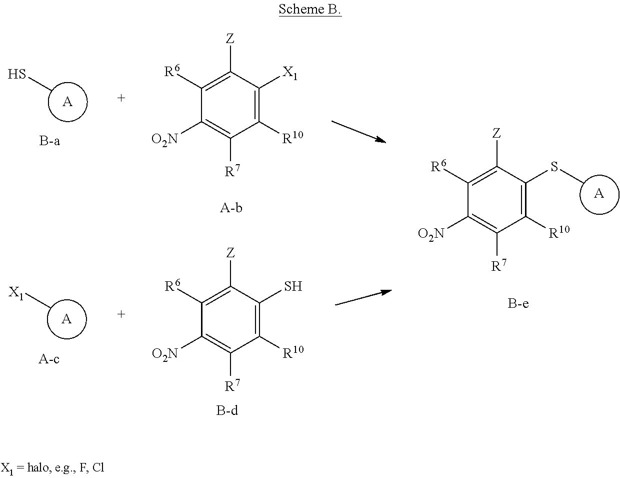

As shown in Scheme B, nucleophilic substitution by thiolated Ring A-containing heterocycles of general formula B-a of nitrosylated benzenes of general formula A-b provides the coupled thioether compounds of general formula B-e. Alternatively, compounds of general formula A-c may be reacted with compounds of general formula B-d to yield the coupled thioether compounds of general formula B-e.

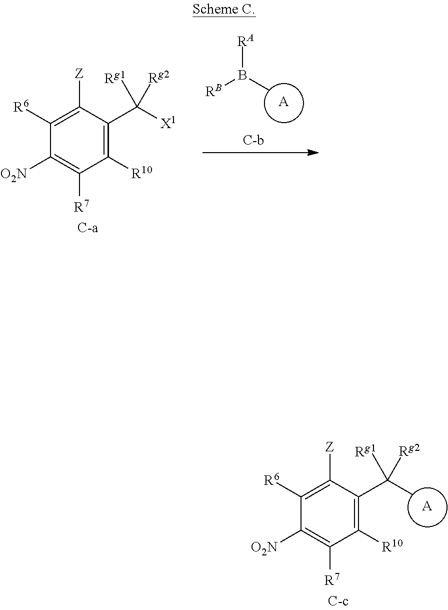

As shown in Scheme C, compounds of general formula C-a can be coupled with a suitable Ring A-substituted boronic acid derivative C-b, wherein R^{A} and R^{B} are independently selected from the group consisting of OH and O—(C₁-C₆ alkyl), or R^{A} and R^{B} are taken together with the boron atom to which they are attached to form a 5-10 membered heterocycle, to afford compounds of general formula C-c.

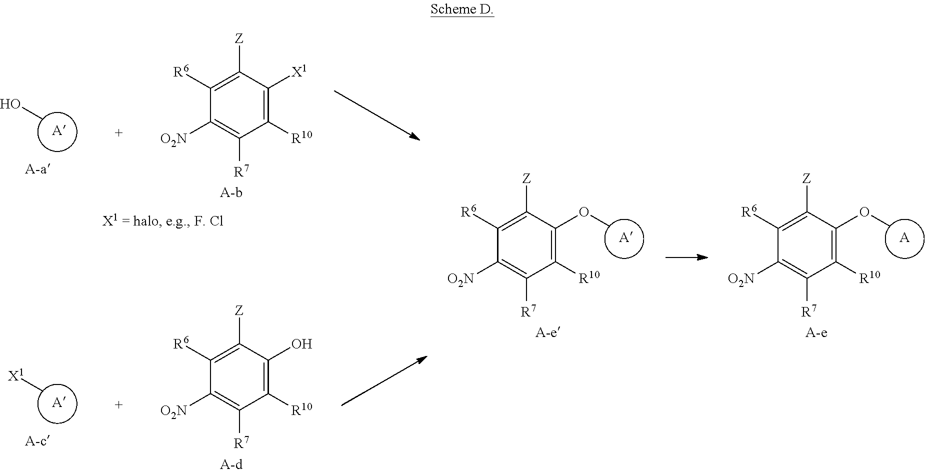

In one variation of Scheme A, as shown in Scheme D, the final Ring A may be prepared or formed from Ring A′, a precursor to Ring A, as part of the synthetic scheme. With reference to Scheme D, nucleophilic substitution by hydroxylated Ring A′-containing heterocycles of general formula A-a′ of nitrosylated benzenes of general formula A-b provides the coupled ether compounds of general formula A-e′. Alternatively, compounds of general formula A-c′ may be reacted with compounds of general formula A-d to yield the coupled ether compounds of general formula A-e′. Ring A′ may be converted to Ring A, for example, by the conversion of formula A-e′ to formula A-e.

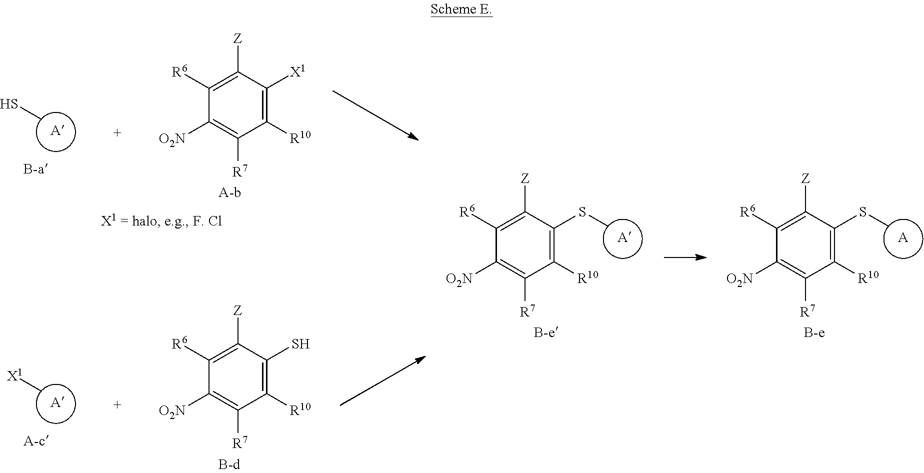

In one variation of Scheme B, as shown in Scheme E, the final Ring A may be prepared or formed from Ring A′, a precursor to Ring A, as part of the synthetic scheme. With reference to Scheme E, nucleophilic substitution by thiolated Ring A′-containing heterocycles of general formula B-a′ of nitrosylated benzenes of general formula A-b provides the coupled thioether compounds of general formula B-e′. Alternatively, compounds of general formula A-c′ may be reacted with compounds of general formula B-d to yield the coupled thioether compounds of general formula B-e′. Ring A′ may be converted to Ring A, for example, by the conversion of formula B-e′ to formula B-e.

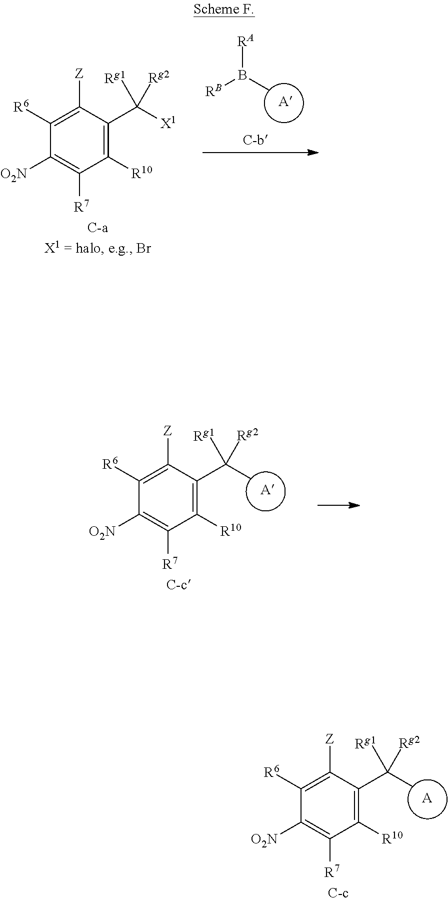

In one variation of Scheme C, as shown in Scheme F, the final Ring A may be prepared or formed from Ring A′, a precursor to Ring A, as part of the synthetic scheme. With reference to Scheme F, compounds of general formula C-a can be coupled with a suitable Ring A′-substituted boronic acid derivative C-b′, wherein R^{A} and R^{B} are independently selected from the group consisting of OH and O—(C₁-C₆ alkyl), or R^{A} and R^{B} are taken together with the boron atom to which they are attached to form a 5-10 membered heterocycle, to afford compounds of general formula C-c′. Ring A′ may be converted to Ring A, for example, by the conversion of formula C-c′ to formula C-c.

Ring A′ may encompass, but is not limited to, monocyclic heteroaryl rings such as pyridyl, pyrimidine, pyrazine. Exemplary reactions to convert Ring A′ to Ring A (in Scheme D, Scheme E, or Scheme F) may include, for example, reacting a monocyclic Ring A′ with a suitable substrate and cyclizing the substrate to form a bicyclic Ring A as shown in Scheme G, Scheme H, or Scheme I below, or adding one or more substituents to Ring A′ to give Ring A.

As shown in Scheme G, compounds of general formula D-a (corresponding to compounds of general formulae A-e′, B-e′, or C-c′) are reacted with N,N-dimethylformamide-dimethyl acetal (DMF-DMA) to give compounds of general formula D-b, and further reacting compounds of general formula D-b with ammonium hydroxide to yield compounds of general formula D-c. Compounds of general formula D-c are further cyclized, for example, in the presence of trifluoroacetic anhydride, to provide compounds of general formula D-d (corresponding to compounds of general formulae A-e, B-e, or C-c) having bicyclic Ring A.

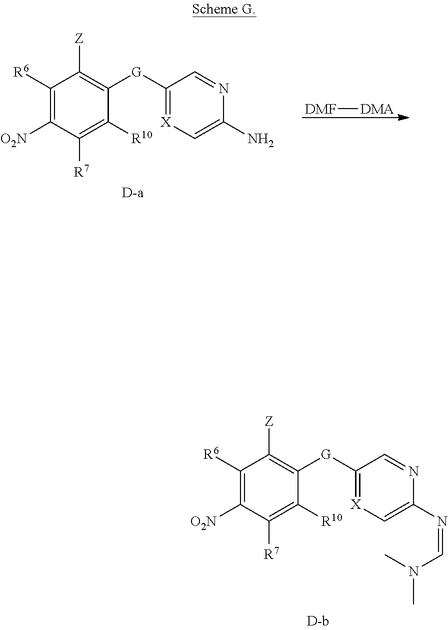

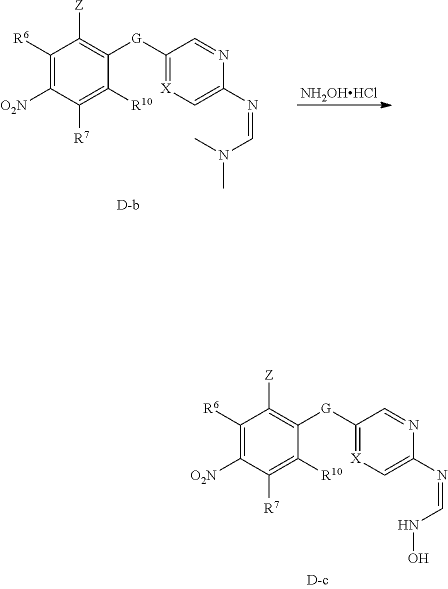

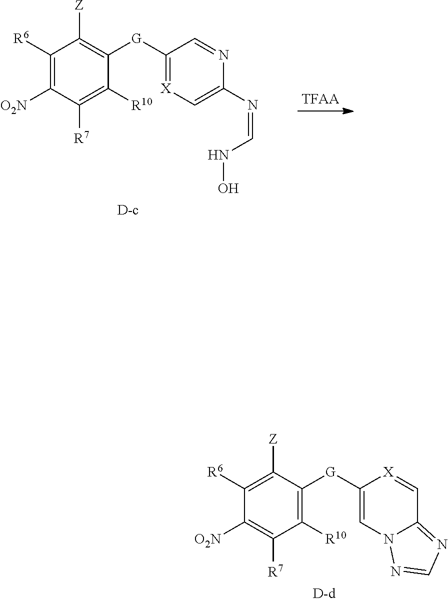

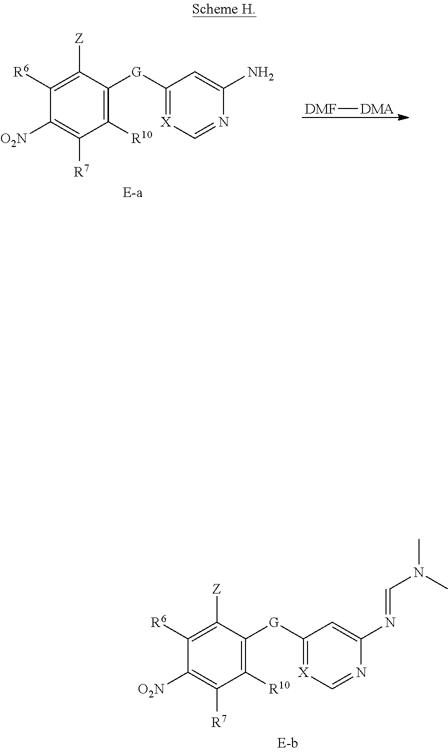

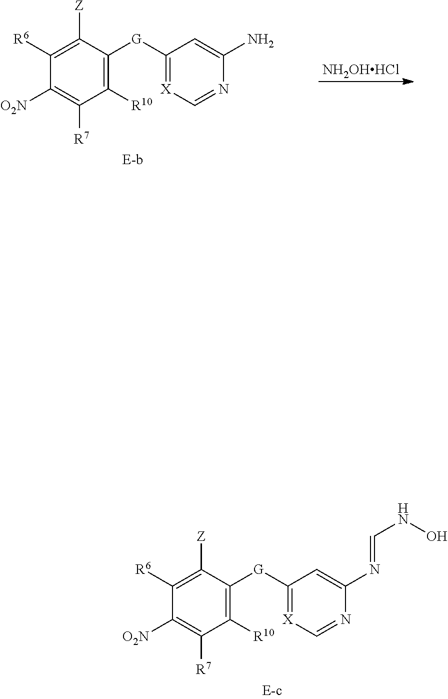

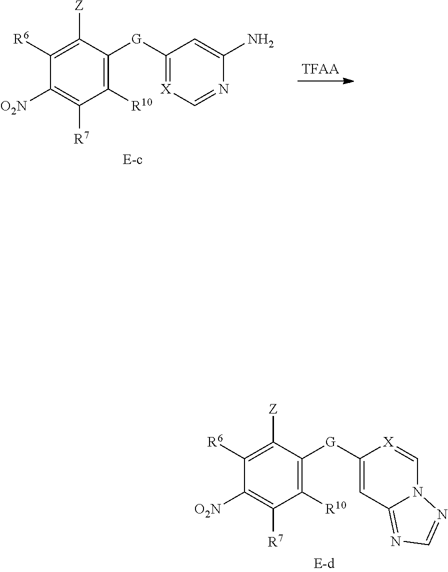

As shown in Scheme H, compounds of general formula E-a (corresponding to compounds of general formulae A-e′, B-e′, or C-c′) are reacted with N,N-dimethylformamide-dimethyl acetal to give compounds of general formula E-b, and further reacting compounds of general formula E-b with ammonium hydroxide to yield compounds of general formula E-c. Compounds of general formula E-c are further cyclized, for example, in the presence of trifluoroacetic anhydride, to provide compounds of general formula E-d (corresponding to compounds of general formulae A-e, B-e, or C-c) having bicyclic Ring A.

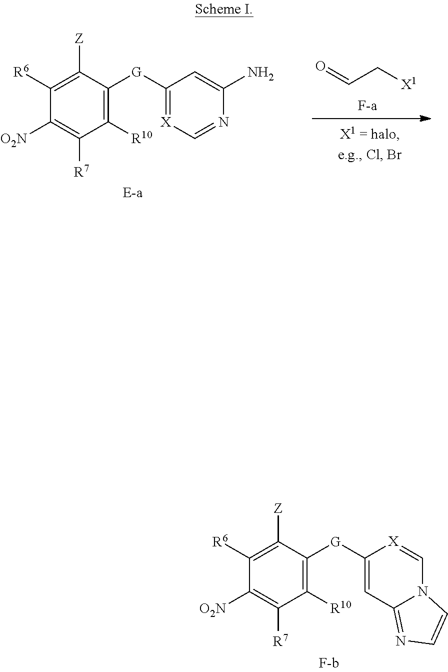

As shown in Scheme I, compounds of general formula E-a (corresponding to compounds of general formulae A-e′, B-e′, or C-c′) are reacted with compounds of general formula F-a to give compounds of general formula F-b (corresponding to compounds of general formulae A-e, B-e, or C-c) having bicyclic Ring A.

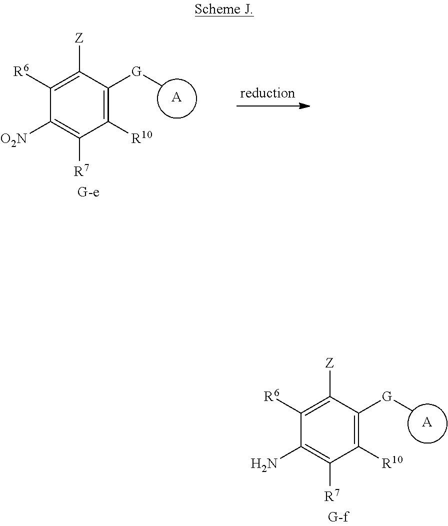

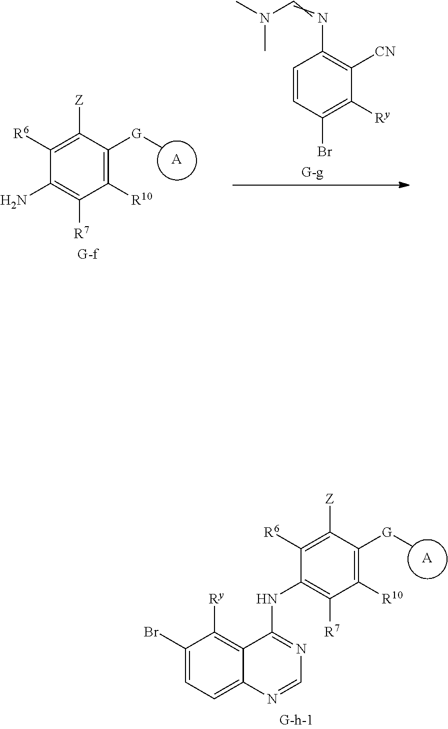

Compounds of formula G-h-1 may be prepared according to the general synthetic scheme shown in Scheme J. The nitrosyl group on the compounds of general formula G-e (corresponding to compounds of general formulae A-e, B-e, or C-c) is reduced (for example, in the presence of H2 and Pd/C) to give compounds of general formula G-f. The compounds of general formula G-f are reacted with compounds of general formula G-g to yield intermediate compounds of general formula G-h-1.

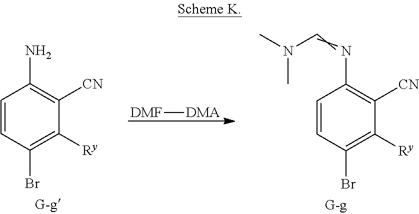

The compounds of formula G-g may be prepared from the reaction of a suitable precursor G-g′ with N,N-dimethylformamide dimethyl acetal (DMF-DMA), as shown in Scheme K above.

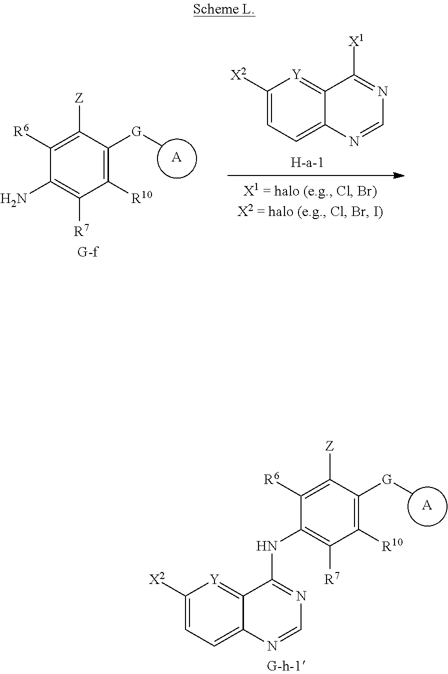

An alternative to the synthesis shown for preparing compounds of general formula G-h-1 provided in Scheme J, Scheme L above shows the synthesis for compounds of general formula G-h-1′. The compounds of general formula G-f are reacted with compounds of general formula H-a-1 to give compounds of general formula G-h-1′. Scheme M and Scheme N depict a similar synthetic approach as Scheme L to give compounds of general formulae G-h-2 and G-h 3, respectively.

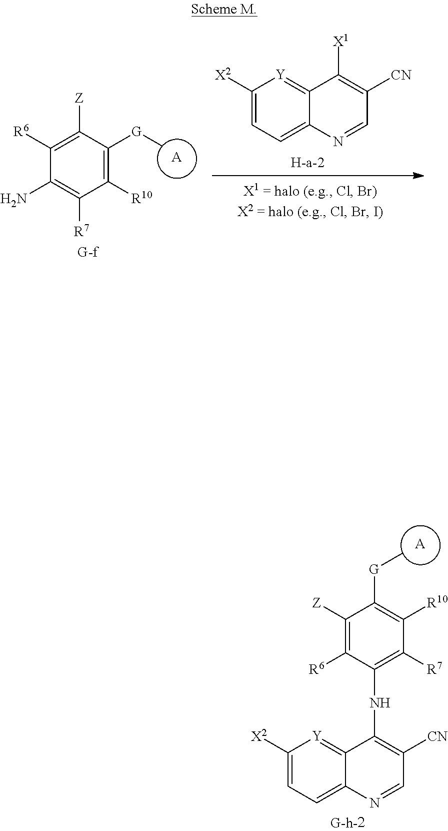

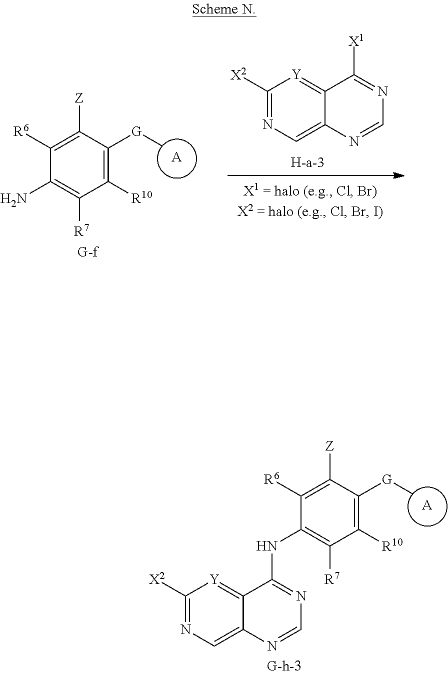

As shown in Scheme O, compounds of general formula G-h (including compounds of formulae G-h-1, G-h-1′, G-h-2, and G-h-3) are reacted with lactam rings of formula G-i to yield compounds of formula (II-a). The lactam compounds of formula G-i may be obtained and/or prepared from commercially available starting materials, as shown in General Scheme P.

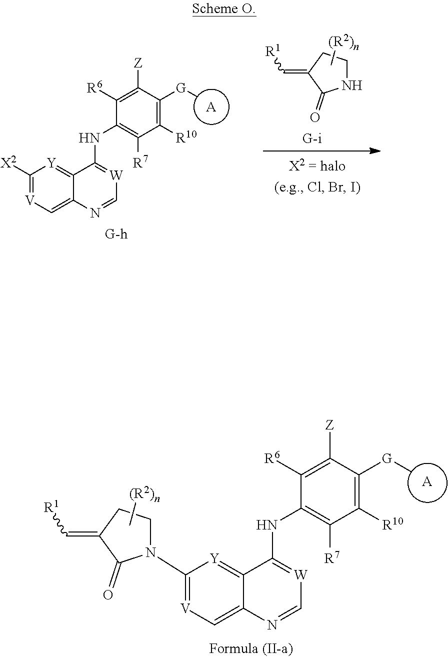

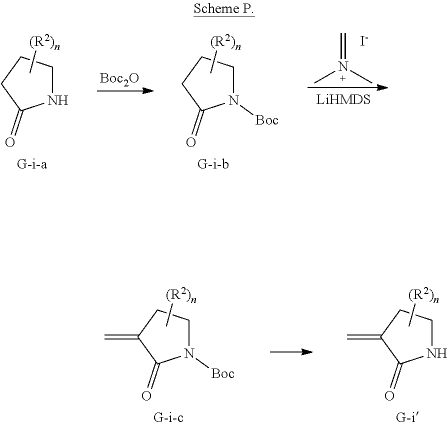

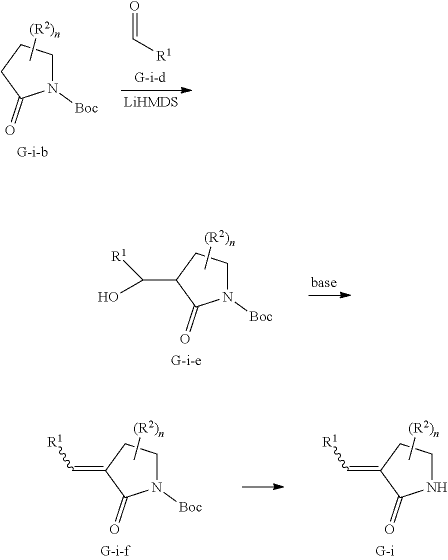

As shown in Scheme P, lactam compounds of general formula G-i-a are protected with a suitable nitrogen protecting moiety (e.g., Boc-group) to give the protected lactam rings of formula G-i-b. The compound of general formula G-i-b may be reacted with Eschenmoser's salt in the presence of a suitable strong non-nucleophilic base, such as LiHMDS, to yield the intermediate compounds of general formula G-i-c. The compounds of general formula G-i-c are deprotected to give the compounds of formula G-i′, wherein R¹ is hydrogen. Alternatively, the lactam compounds of formula G-i-b may be reacted with an R¹-substituted carbonyl compound of general formula G-i-d in the presence of a suitable strong non-nucleophilic base, such as LiHMDS, to yield the intermediate hydroxymethylated compounds of general formula G-i-e. The compounds of general formula G-i-e are treated with a base in an elimination reaction to give the intermediate compound of general formula G-i-f. The compound of general formula G-i-f is deprotected to give the final compound of general formula G-i. Similar synthetic methods to those shown in Scheme P for five-membered lactam intermediates G-i and G-i′ (p=0) may be employed to obtain six-membered lactam intermediates G-ii and G-ii′ (p=1), as shown in Scheme Q. The six-membered lactam intermediates G-ii and G-ii′ (p=1) may be used for preparing compounds of formula (II-b) via synthetic methods analogous to Scheme O.

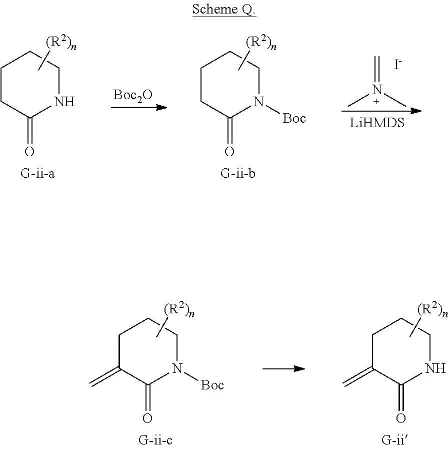

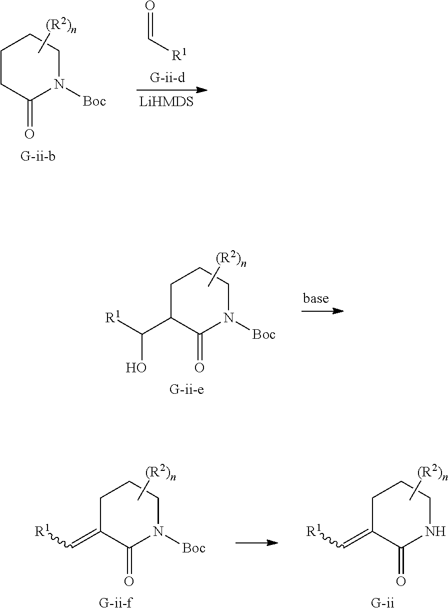

In still other variations of Scheme M, N and O, compounds of formula (II-c) and (II-d) may be prepared as shown in Schemes R, S, and T below.

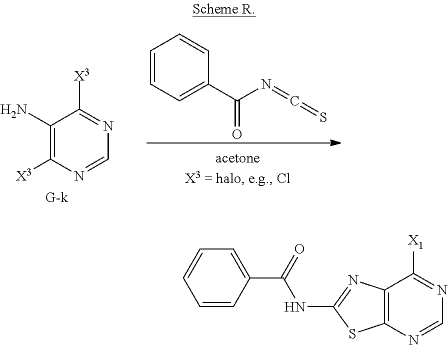

As shown in Scheme R, thiazolo[5,4-d]pyrimidine compounds of formula H-a-4 may be prepared by reacting a suitable pyrimidine of formula G-k with a benzoyl isothiocyanate to form a benzoyl-protected thiazolo[5,4-d]pyrimidine H-a-4.

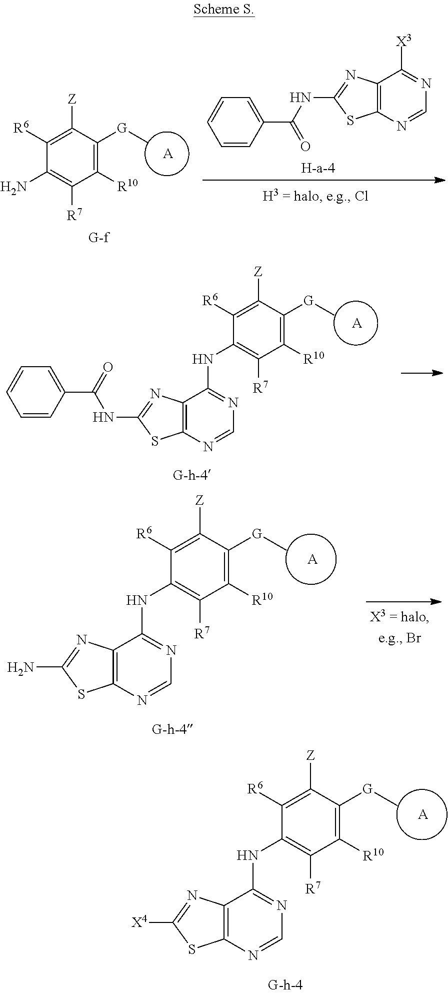

As further shown in Scheme S, the benzoyl-protected thiazolo[5,4-d]pyrimidine of formula H-a-4 may be reacted with a compound of formula G-f, analogous to Schemes L, M and N, to provide an intermediate compound of G-h-4′, which may be subsequently deprotected to a compound of formula G-h-4″, and halogenated to give an intermediate of formula G-h-4. In Scheme T, analogous to Scheme O, the compound of G-h-4 may be subsequently reacted with lactam rings of formula G-i to yield compounds of formula (II-c). Compounds of formula (II-d) may be prepared by employing six-membered lactam precursor G-ii (p=1) in place of the five-membered lactam G-i (p=0) of Scheme T.

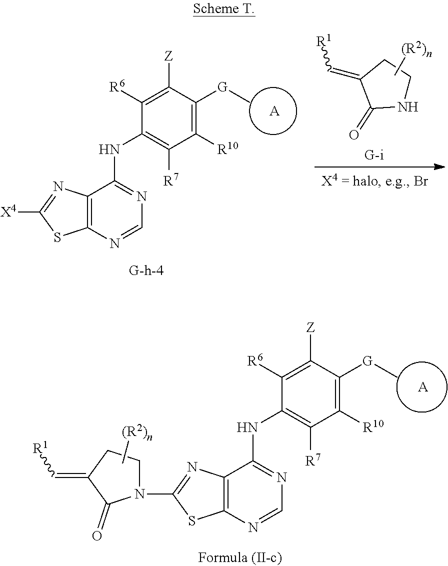

It should be recognized that the present disclosure also provides for intermediates of and methods of synthesizing the compounds of (I), (II-a), (II-b), (II-c), (II-d), (III), (IV), (I-a), (I-b), (I-c), (I-d), (I-e), (I-f), (I-g), (I-h), (I-i), (I-a-1), (I-a-2), (I-b-1), (I-b-2), (I-c-1), (I-c-2), (I-d-1), (I-d-2), (I-e-1), (I-e-2), (I-f-1), (I-f-2), (I-g-1), (I-g-2), (I-h-1), (I-h-2), (I-i-1), (I-i-2), (I-j-1), (I-j 2), (I-j-3), (I-j-4), (I-j-5), (I-j-6), (I-j-7), (I-j-8), (I-j-9), (I j-10), (I j-11), (I-j-12), (I-j-13), (I j-14), (I-j-15), (I-j-16), (I-j-17), (I-j-18), (I-k-1), (I-k-2), (I-k-3), (I-k-4), (I-k-5), (I-k-6), (I-k-7), (I-k-8), (I-k-9), (I-k-10), (I-k-11), (I-k-12), (I-k-13), (I-k-14), (I-k-15), (I-k-16), (I-k-17), (I-k-18), (I-l-1), (I-l-2), (I-l-3), (I-l-4), (I-l-5), (I-l-6), (I-l-7), (I-l-8), (I-l-9), (I-l-10), (I-l-11), (I-l-12), (I-l-13), (I-l-14), (I-l-15), (I-l-16), (I-l-17), or (I-l-18), described herein. In one aspect, provided herein are synthetic methods as described in any one of Schemes A through T above or in the examples below. In another aspect, provided herein are general intermediates as described in any one of Schemes A through T above, or compound-specific intermediates as described in the examples below. It should be further recognized that the present disclosure also provides for synthetic methods comprising any individual step or combination of individual process steps, or compositions of synthetic intermediates and/or reaction products as described herein

### EXAMPLES

It is understood that the present disclosure has been made only by way of example, and that numerous changes in the combination and arrangement of parts can be resorted to by those skilled in the art without departing from the spirit and scope of present disclosure.

The chemical reactions in the Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by appropriately protecting interfering groups, by utilizing other suitable reagents known in the art other than those described, or by making routine modifications of reaction conditions, reagents, and starting materials. Alternatively, other reactions disclosed herein or known in the art will be recognized as having applicability for preparing other compounds of the present disclosure.

Abbreviations used in the Examples include the following: ACN: acetonitrile; AcOH: acetic acid; AcOK: potassium acetate; BSA: bovine serum albumin; DBU: 1,8-Diazabicyclo[5.4.0]undec-7-ene; DCM: dichloromethane; DIEA: diisopropylethylamine; DMAP: 4-dimethylaminopyridine; DMF: dimethylformamide; DMF-DMA: dimethylformamide-dimethyl acetal; DMSO: dimethyl sulfoxide; DTT: dithiothreitol; ESI: electrospray ionization; EGTA: ethylene glycol-bis(β-aminoethyl ether)-N,N,N′,N′-tetraacetic acid; EtOAc: ethyl acetate; EtOH: ethanol or ethyl alcohol; ¹H NMR: proton nuclear magnetic resonance; HATU: 1-[Bis(dimethylamino)methylene]-1H-1,2,3-triazolo[4,5-b]pyridinium 3-oxide hexafluorophosphate (Hexafluorophosphate Azabenzotriazole Tetramethyl Uronium); LCMS: liquid chromatography-mass spectrometry; LiHMDS: lithium hexamethyldisilazide; MeOH: methanol or methyl alcohol; n-BuLi: n-butyllithium; MsCl: mesyl chloride; NMP: N-methylpyrrolidone; PBS: phosphate-buffered saline; PBST: PBS with Tween 20; Py: pyridine; TEA: triethylamine; TFA: trifluoroacetic acid; TFAA: trifluoroacetic anhydride; THF: tetrahydrofuran; and TLC: thin-layer chromatography.

### Example S1. Synthesis of 1-[4-[(3-methyl-4-[[1,2,4]triazolo[1,5-a]pyridin-7-yloxy]phenyl)amino]quinazolin-6-yl]-3-methylidenepyrrolidin-2-one (Compound 1)

### Step 1. Synthesis of tert-butyl 3-methylidene-2-oxopyrrolidine-1-carboxylate

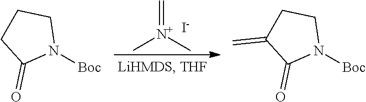

To a solution of tert-butyl 2-oxopyrrolidine-1-carboxylate (6.0 g, 32.39 mmol) in THF (50.0 mL) was added LiHMDS (7.05 g, 42.111 mmol) dropwise at −78° C. under N₂. The mixture was stirred at −78° C. for 1 h. Then Eschenmoser's salt (9.0 g, 48.59 mmol) was added to the mixture. The mixture was stirred at −78° C. for another 1 h. After the reaction was completed, the reaction mixture was quenched with NH₄Cl (aq.) and extracted with ethyl acetate. The combined organic layer was washed with brine, dried over anhydrous sodium sulfate and filtered. The filtrate was concentrated under reduced pressure. The residue was used in the next step without further purification. The residue was diluted in EtOH (50.0 mL) and allyl bromide (31.8 g, 263.03 mmol). Na₂CO₃ (20.3 g, 192.09 mmol) was successively added. The mixture was stirred at room temperature for 3 d. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (45/55, v/v) to afford tert-butyl 3-methylidene-2-oxopyrrolidine-1-carboxylate (4.3 g, 67%) as a yellow solid. LCMS (ESI, m/z): [M+H]⁺=198.1.

### Step 2. Synthesis of 3-methylidenepyrrolidin-2-one

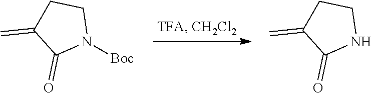

To a solution of tert-butyl 3-methylidene-2-oxopyrrolidine-1-carboxylate (4.3 g, 21.8 mmol) in CH₂Cl₂ (20.0 mL) was added TFA (10 mL). The resulting mixture was stirred at room temperature for 1 h. After the reaction was completed, the resulting mixture was concentrated under reduced pressure to afford 3-methylidenepyrrolidin-2-one (2.0 g, crude) as a yellow oil. LCMS (ESI, m/z): [M+H]⁺=98.1.

### Step 3. Synthesis of N′-(4-bromo-2-cyanophenyl)-N,N-dimethylformimidamide

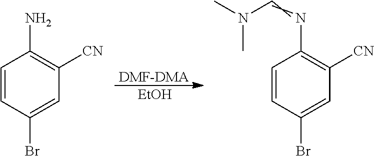

To a solution of 2-amino-5-bromobenzonitrile (3.0 g, 15.22 mmol) in EtOH (10.0 mL) was added DMF-DMA (3.1 g, 25.88 mmol) at room temperature. The resulting mixture was stirred at 75° C. for 3 h. After the reaction was completed, the resulting mixture was concentrated under vacuum. The residue was purified by flash column chromatography with petroleum ether/ethyl acetate (1/9, v/v) to afford N′-(4-bromo-2-cyanophenyl)-N,N-dimethylformimidamide (3.5 g, 91%) as a white solid. LCMS (ESI, m/z): [M+H]⁺=252.0.

### Step 4. Synthesis of 6-bromo-N-(3-methyl-4-[[1,2,4]triazolo[1,5-a]pyridin-7-yloxy]phenyl)quinazolin-4-amine

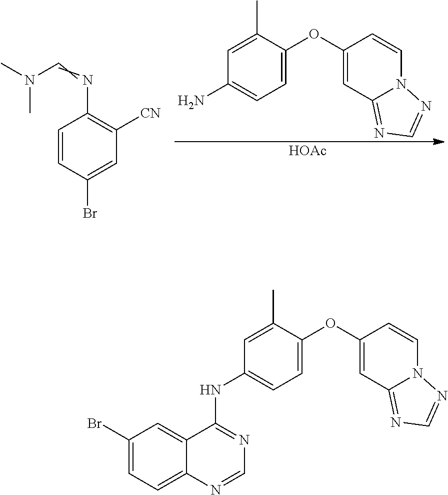 [SMILES: CN(C)C=Nc1ccc(Br)cc1C#N ; Cc1cc(N)ccc1Oc1ccn2ncnc2c1 ; Cc1cc(Nc2ncnc3ccc(Br)cc23)ccc1Oc1ccn2ncnc2c1 ; "HOAc"]

To a solution of 3-methyl-4-[[1,2,4]triazolo[1,5-a]pyridin-7-yloxy]aniline (500.0 mg, 2.08 mmol) in acetic acid (10.0 mL) was added N′-(4-bromo-2-cyanophenyl)-N,N-dimethylformimidamide (524.7 mg, 2.08 mmol). The resulting mixture was stirred at 85° C. for 2 h. After the reaction was completed, the mixture was evaporated in vacuo. The residue was purified by reverse phase flash column chromatography with acetonitrile/water (81/19, v/v) to afford 6-bromo-N-(3-methyl-4-[[1,2,4]triazolo[1,5-a]pyridin-7-yloxy]phenyl)quinazolin-4-amine (400.0 mg, 42%) as a yellow solid. LCMS (ESI, m/z): [M+H]⁺=447.0.

### Step 5. Synthesis of 1-[4-[(3-methyl-4-[[1,2,4]triazolo[1,5-a]pyridin-7-yloxy]phenyl)amino]quinazolin-6-yl]-3-methylidenepyrrolidin-2-one (Compound 1)

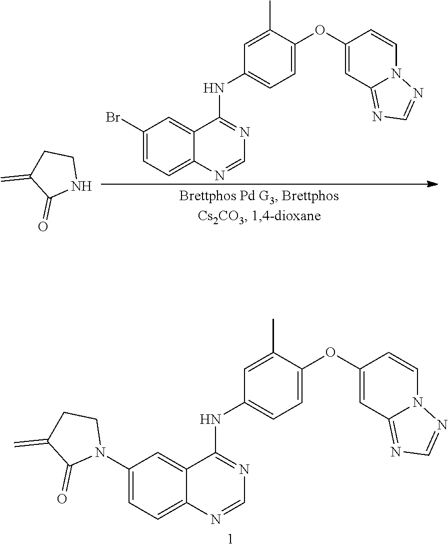

To a solution of 6-bromo-N-(3-methyl-4-[[1,2,4]triazolo[1,5-a]pyridin-7-yloxy]phenyl)quinazolin-4-amine (300.0 mg, 0.67 mmol) in 1,4-dioxane (5.0 mL) was added 3-methylidenepyrrolidin-2-one (195.4 mg, 2.01 mmol), Cs₂CO₃ (1.31 g, 4.02 mmol), BrettPhos (72.0 mg, 0.13 mmol) and Brettphos Pd G3 (60.8 mg, 0.07 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (85/15, v/v) and then purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 22% B to 46 B % in 7 min; 254 nm) to afford 1-[4-[(3-methyl-4-[[1,2,4]triazolo[1,5-a]pyridin-7-yloxy]phenyl)amino]quinazolin-6-yl]-3-methylidenepyrrolidin-2-one (Compound 1) (68.8 mg, 22%) as a yellow solid. LCMS (ESI, m/z): [M+H]⁺=463.9. ¹H NMR (300 MHz, DMSO-d₆): δ 9.87 (s, 1H), 8.95 (d, J=7.5 Hz, 1H), 8.72-8.68 (m, 1H), 8.59 (s, 1H), 8.39-8.36 (m, 2H), 7.86-7.80 (m, 3H), 7.26-7.22 (m, 1H), 7.06-7.03 (m, 1H), 6.82 (d, J=2.4 Hz, 1H), 6.00 (s, 1H), 5.56 (s, 1H), 4.09-4.04 (m, 2H), 3.01-2.96 (m, 2H), 2.22 (s, 3H).

### Example S2. Synthesis of 5-[4-[(3-methyl-4-[[1,2,4]triazolo[1,5-a]pyridin-7-yloxy]phenyl)amino]quinazolin-6-yl]-7-methylidene-5-azaspiro[2.4]heptan-6-one (Compound 2)

### Step 1. Synthesis of tert-butyl 6-oxo-5-azaspiro[2.4]heptane-5-carboxylate

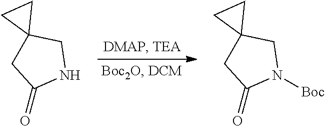 [SMILES: O=C1CC2(CC2)CN1 ; CC(C)(C)OC(=O)N1CC2(CC2)CC1=O]

To a solution of 5-azaspiro[2.4]heptan-6-one (500.0 mg, 4.50 mmol) in CH₂Cl₂ (10.0 mL) added TEA (1.4 g, 13.49 mmol), DMAP (11.0 mg, 0.09 mmol) and Boc₂O (981.8 mg, 4.50 mmol) at room temperature. The resulting mixture was stirred at room temperature for 2 h. After the reaction was completed, the resulting mixture was concentrated under reduced pressure The residue was purified by flash column chromatography with petroleum ether/ethyl acetate (75/25, v/v) to afford tert-butyl 6-oxo-5-azaspiro[2.4]heptane-5-carboxylate (500.0 mg, 52%) as a white solid. LCMS (ESI, m/z): [M+H]⁺=212.1.

### Step 2. Synthesis of tert-butyl 7-methylidene-6-oxo-5-azaspiro[2.4]heptane-5-carboxylate

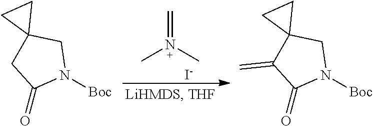

To a solution of tert-butyl 6-oxo-5-azaspiro[2.4]heptane-5-carboxylate (500.0 mg, 2.37 mmol) in THE (20.0 mL) was added LiHMDS (514.8 mg, 3.08 mmol) dropwise at −78° C. under N₂. The mixture was stirred at −78° C. for 1 h. Then Eschenmoser's salt (656.8 mg, 3.55 mmol) was added to the mixture at −78° C. The mixture was stirred at −78° C. for another 1 h and then warmed up to 0° C. After the reaction was completed, the reaction mixture was quenched with aq. NH₄Cl at room temperature. The resulting mixture was extracted with ethyl acetate. The combined organic layer was washed with brine, dried over anhydrous sodium sulfate and filtered. The filtrate was concentrated under reduced pressure. The residue was dissolved in EtOH (20.0 mL). Allyl bromide (2.3 g, 19.22 mmol) and Na₂CO₃ (1.5 g, 13.97 mmol) were successively added to the mixture. The mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (78/22, v/v) to afford tert-butyl 7-methylidene-6-oxo-5-azaspiro[2.4]heptane-5-carboxylate (500.0 mg, 94%) as a white solid. LCMS (ESI, m/z): [M+H]⁺=224.1.

### Step 3. Synthesis of 7-methylidene-5-azaspiro[2.4]heptan-6-one

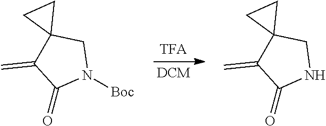

To a solution of tert-butyl 7-methylidene-6-oxo-5-azaspiro[2.4]heptane-5-carboxylate (500.0 mg, 2.24 mmol) in CH₂Cl₂ (5.0 mL) was added TFA (2.0 mL) at room temperature. The resulting mixture was stirred at room temperature for 1 h. After the reaction was completed, the mixture was basified to pH 8 with aq. NaHCO₃ and then extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 7-methylidene-5-azaspiro[2.4]heptan-6-one (200.0 mg, 72%) as a yellow solid. LCMS (ESI, m/z): [M+H]⁺=124.0.

### Step 4. Synthesis of 5-[4-[(3-methyl-4-[[1,2,4]triazolo[1,5-a]pyridin-7-yloxy]phenyl)amino]quinazolin-6-yl]-7-methylidene-5-azaspiro[2.4]heptan-6-one (Compound 2)

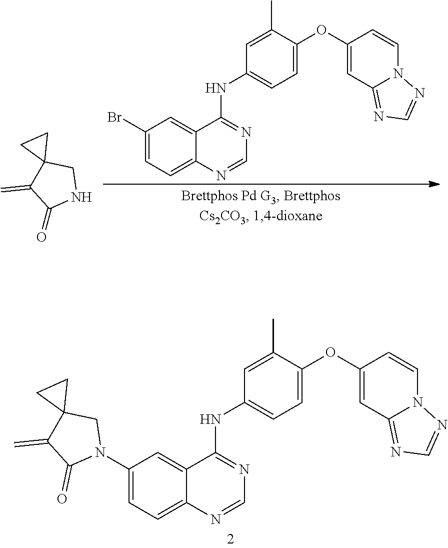

To a mixture of 6-bromo-N-(3-methyl-4-[[1,2,4]triazolo[1,5-a]pyridin-7-yloxy]phenyl)quinazolin-4-amine (100.0 mg, 0.22 mmol) and 7-methylidene-5-azaspiro[2.4]heptan-6-one (82.6 mg, 0.67 mmol) in 1,4-dioxane (5.0 mL) was added Cs₂CO₃ (437.1 mg, 1.34 mmol), BrettPhos (24.0 mg, 0.05 mmol) and Brettphos Pd G3 (20.3 mg, 0.02 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (9/1, v/v) and then purified by Prep-HPLC with the following conditions Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 26% B to 56% B in 9 min, 56 B in 9 min, 254 nm) to afford 5-[4-[(3-methyl-4-[[1,2,4]triazolo[1,5-a]pyridin-7-yloxy]phenyl)amino]quinazolin-6-yl]-7-methylidene-5-azaspiro[2.4]heptan-6-one (Compound 2) (51.5 mg, 47%) as a white solid. LCMS (ESI, m/z): [M+H]⁺=490.1. ¹H NMR (300 MHz, DMSO-d₆): δ 9.86 (s, 1H), 8.95 (d, J=7.5 Hz, 1H), 8.72-8.69 (m, 1H), 8.60 (s, 1H), 8.39 (s, 1H), 8.36 (d, J=2.1 Hz, 1H), 7.88-7.81 (m, 3H), 7.26-7.23 (m, 1H), 7.06-7.03 (m, 1H), 6.81 (d, J=2.4 Hz, 1H), 5.72 (s, 1H), 5.11 (s, 1H), 4.08 (s, 2H), 2.21 (s, 3H), 1.25-1.22 (m, 2H), 1.15-1.11 (m, 2H).

### Example S3: Synthesis of 1-[4-([3-methyl-4 [(6-methylpyridin-3-yl)oxy]phenyl]amino)quinazolin-6-yl]-3-methylidenepyrrolidin-2-one (Compound 3)

### Step 1. Synthesis of 2-methyl-5-(2-methyl-4-nitrophenoxy)pyridine

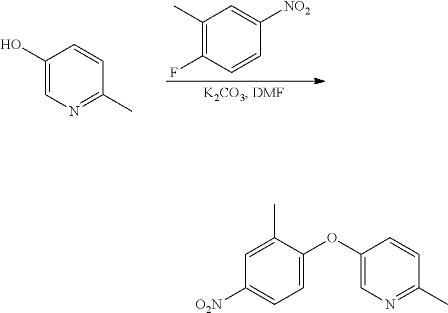

To a solution of 6-methylpyridin-3-ol (5.0 g, 45.82 mmol) in DMF (30.0 mL) was added 1-fluoro-2-methyl-4-nitrobenzene (8.5 g, 54.98 mmol) and K₂CO₃ (12.7 g, 91.64 mmol) at room temperature. The resulting mixture was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse flash column chromatography with acetonitrile/water (55/45, v/v) to afford 2-methyl-5-(2-methyl-4-nitrophenoxy)pyridine (4.0 g, 35%) as a yellow oil. LCMS (ESI, m/z): [M+H]⁺=245.1.

### Step 2a. Synthesis of 3-methyl-4-[(6-methylpyridin-3-yl)oxy]aniline

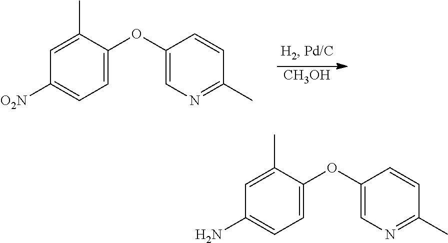

To a solution of 2-methyl-5-(2-methyl-4-nitrophenoxy)pyridine (4.0 g, 16.38 mmol) in CH₃OH (30.0 mL) was added Pd/C (1.7 g, dry) at room temperature. The resulting mixture was stirred at room temperature for 16 h under H₂. After the reaction was completed, the resulting mixture was filtered. The filtrate was concentrated under reduced pressure. The residue was purified by flash column chromatography with petroleum ether/ethyl acetate (36/64, v/v) to afford 3-methyl-4-[(6-methylpyridin-3-yl)oxy]aniline (3.5 g, 99%) as a white solid. LCMS (ESI, m/z): [M+H]⁺=215.1.

### Step 2b. Alternative Synthesis of 3-methyl-4-[(6-methylpyridin-3-yl)oxy]aniline

In an alternative to Step 2a: To a solution of 2-methyl-5-(2-methyl-4-nitrophenoxy)pyridine (30.10 g, 123.2 mmol) in MeOH (300.0 mL) was added Pd/C (3.0 g, dry) at 0° C. and was stirred for 19 h at room temperature under H2 atmosphere. After the reaction was completed, the resulting mixture was filtered. The filtrate was concentrated under reduced pressure to afford 27.1 g (crude) of 3-methyl-4-((6-methylpyridin-3-yl)oxy)aniline as a yellow solid. LCMS (ESI, m/z): [M+H]⁺=215.

### Step 3. Synthesis of 6-bromo-N-[3-methyl-4-[(6-methylpyridin-3-yl)oxy]phenyl]quinazolin-4-amine

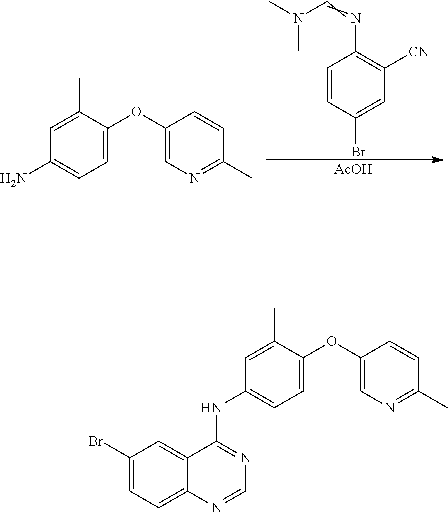 [SMILES: "AcOH" ; Cc1ccc(Oc2ccc(N)cc2C)cn1 ; CN(C)C=Nc1ccc(Br)cc1C#N ; Cc1ccc(Oc2ccc(Nc3ncnc4ccc(Br)cc34)cc2C)cn1]

To a solution of 3-methyl-4-[(6-methylpyridin-3-yl)oxy]aniline (300.0 mg, 1.40 mmol) in HOAc (5.0 mL) was added N′-(4-bromo-2-cyanophenyl)-N,N-dimethylformimidamide (353.0 mg, 1.40 mmol) at room temperature. The resulting mixture was stirred at 85° C. for 2 h. After the reaction was completed, the resulting mixture was concentrated under reduced pressure. The residue was purified by flash column chromatography with CH₂Cl₂/CH₃OH (9/1, v/v) to afford 6-bromo-N-[3-methyl-4-[(6-methylpyridin-3-yl)oxy]phenyl]quinazolin-4-amine (500.0 mg, 84%) as a yellow oil. LCMS (ESI, m/z): [M+H]⁺=421.1.

### Step 4. Synthesis of 1-[4-([3-methyl-4-[(6-methylpyridin-3-yl)oxy]phenyl]amino)quinazolin-6-yl]-3-methylidenepyrrolidin-2-one (Compound 3)

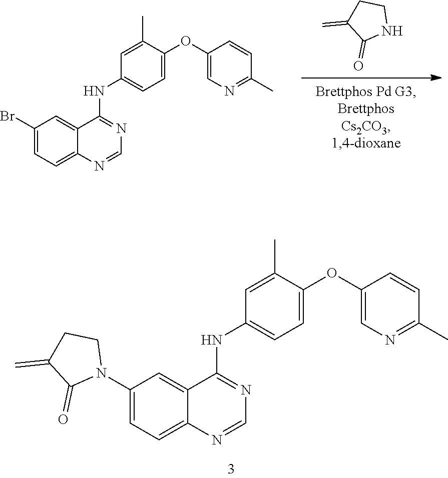

To a solution of 6-bromo-N-[3-methyl-4-[(6-methylpyridin-3-yl)oxy]phenyl]quinazolin-4-amine (300.0 mg, 0.71 mmol) in 1,4-dioxane (5.0 mL) was added 3-methylidenepyrrolidin-2-one (207.5 mg, 2.14 mmol), Cs₂CO₃ (1.39 g, 4.27 mmol), BrettPhos (76.4 mg, 0.14 mmol) and Brettphos Pd G3 (64.6 mg, 0.07 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (88/12, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 27% B to 57% B in 10 min; 254 nm) to afford 1-[4-([3-methyl-4-[(6-methylpyridin-3-yl)oxy]phenyl]amino)quinazolin-6-yl]-3-methylidenepyrrolidin-2-one (Compound 3)(43.0 mg, 13%) as a white solid. LCMS (ESI, m/z): [M+H]⁺=438.2. ¹H NMR (300 MHz, DMSO-d₆): δ 9.80 (s, 1H), 8.71-8.67 (m, 1H), 8.55 (s, 1H), 8.34 (d, J=2.1 Hz, 1H), 8.20 (s, 1H), 7.83 (d, J=9.3 Hz, 1H), 7.73-7.66 (m, 2H), 7.28-7.21 (m, 2H), 6.99 (d, J=8.7 Hz, 1H), 6.00 (s, 1H), 5.55 (s, 1H), 4.08-4.04 (m, 2H), 3.01-2.96 (m, 2H), 2.45 (s, 3H), 2.28 (s, 3H).

### Example S4: Synthesis of 1-[4-[(4-[imidazo[1,2-a]pyridin-7 yloxy]-3-methylphenyl)amino]quinazolin-6-yl]-3-methylidenepyrrolidin-2-one (Compound 4)

### Step 1. Synthesis of N′-(4-bromo-2-cyanophenyl)-N,N-dimethylformimidamide

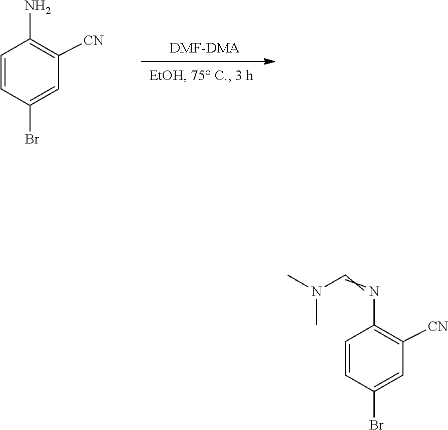

A mixture of 2-amino-5-bromobenzonitrile (10.00 g, 50.752 mmol, 1.00 equiv) and DMF-DMA (12.10 g, 101.504 mmol, 2.00 equiv) in EtOH (50 mL) was stirred at 75° C. for 3 h and then concentrated under vacuum. The residue was purified by trituration with EtOH (20 mL) to afford N′-(4-bromo-2-cyanophenyl)-N,N-dimethylformimidamide (9.8 g, 76%) as a white solid. ¹H NMR (300 MHz, DMSO-d₆) δ 7.98 (s, 1H), 7.81 (d, J=2.4 Hz, 1H), 7.63 (dd, J=8.7, 2.4 Hz, 1H), 7.13 (d, J=8.7 Hz, 1H), 3.08 (s, 3H), 2.99 (s, 3H), MS (ESI, m/z): 252, 254 (M+H)⁺

### Step 2. Synthesis of imidazo[1,2-a]pyridin-7-ol

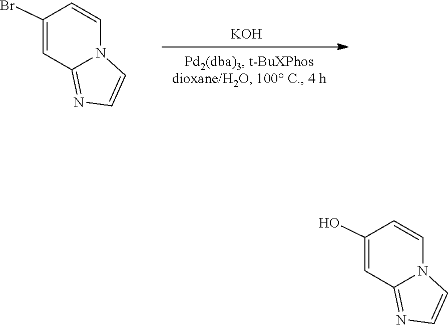 [SMILES: Brc1ccn2ccnc2c1 ; "KOH" ; Oc1ccn2ccnc2c1]

To a stirred solution of 7-bromoimidazo[1,2-a]pyridine (15.00 g, 76.129 mmol, 1.00 equiv) in dioxane/H₂O (150 mL/30 mL) at ambient temperature was added KOH (12.81 g, 228.386 mmol, 3 equiv), Pd₂(dba)₃ (6.97 g, 7.613 mmol, 0.1 equiv), and t-BuXPhos (0.32 g, 0.761 mmol, 0.01 equiv) under N₂ atmosphere. The resulting mixture was stirred at 100° C. for 4 h and then diluted with EtOAc and extracted with H₂O. The aqueous layer was concentrated under vacuum. The residue was purified by flash chromatography on silica gel (0-5% MeOH in DCM) to afford imidazo[1,2-a]pyridin-7-ol (7.5 g, 62%) as a white solid. ¹H NMR (300 MHz, DMSO-d₆) δ 8.33 (dd, J=7.2, 0.9 Hz, 1H), 7.68 (dd, J=1.5, 0.9 Hz, 1H), 7.34 (d, J=1.5 Hz, 1H), 6.70-6.63 (m, 1H), 6.53 (dd, J=7.2, 2.4 Hz, 1H). MS (ESI, m/z): 135 (M+H)+.

### Step 3. Synthesis of 7-(2-methyl-4-nitrophenoxy)imidazo[1,2-a]pyridine

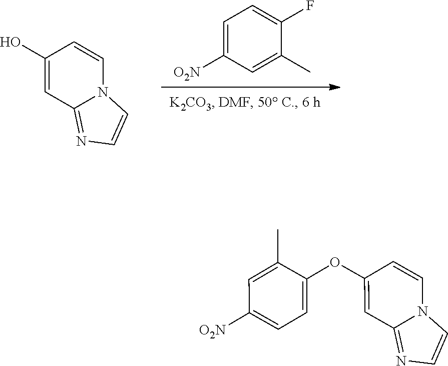 [SMILES: Oc1ccn2ccnc2c1 ; Cc1cc([N+](=O)[O-])ccc1F ; Cc1cc([N+](=O)[O-])ccc1Oc1ccn2ccnc2c1]

A mixture of imidazo[1,2-a]pyridin-7-ol (7.5 g, 55.913 mmol, 1.00 equiv), 1-fluoro-2-methyl-4-nitrobenzene (8.67 g, 55.913 mmol, 1.00 equiv), and K₂CO₃ (15.45 g, 111.826 mmol, 2.00 equiv) in N,N-dimethylformamide (75 mL) was stirred at 50° C. for 6 h and then diluted with H₂O (200 mL). The precipitate solid was collected by filtration, rinsed with water 3 times, and dried in an oven at 50° C. for 16 h. The crude product was purified by flash chromatography on silica gel (0-5% MeOH in DCM) to afford 7-(2-methyl-4-nitrophenoxy)imidazo[1,2-a]pyridine (5 g, 31%) as a white solid. ¹H NMR (400 MHz, DMSO-d₆) δ 8.64 (dd, J=7.2, 0.8 Hz, 1H), 8.29 (dd, J=2.8, 0.8 Hz, 1H), 8.10 (dd, J=8.8, 2.8 Hz, 1H), 7.94 (t, J=1.0 Hz, 1H), 7.55 (d, J=1.2 Hz, 1H), 7.17-7.07 (m, 2H), 6.84 (dd, J=7.6, 2.8 Hz, 1H), 2.38 (s, 3H). MS (ESI, m/z): 270 (M+H)+.

### Step 4. Synthesis of 4-[imidazo[1,2-a]pyridin-7-yloxy]-3-methylaniline

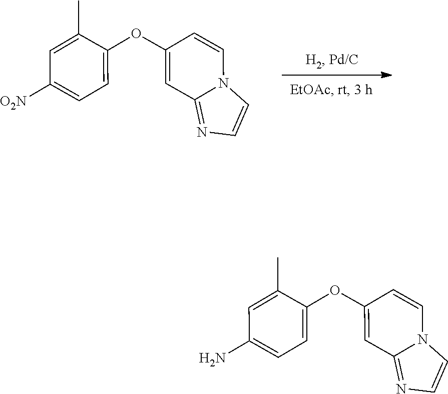 [SMILES: Cc1cc([N+](=O)[O-])ccc1Oc1ccn2ccnc2c1 ; Cc1cc(N)ccc1Oc1ccn2ccnc2c1]

A mixture of 7-(2-methyl-4-nitrophenoxy)imidazo[1,2-a]pyridine (5.00 g, 18.569 mmol, 1.00 equiv) and palladium carbon (wet, 10%, 1.5 g, 30% w/w) in EtOAc (50.00 mL) was stirred at ambient temperature for 4 h under H₂ atmosphere (˜3 atm) and then filtered through a pad of celite. The filtrate was concentrated under vacuum to afford 4-[imidazo[1,2-a]pyridin-7-yloxy]-3-methylaniline (crude, 5 g) as an off-white solid, which was used for the next step without further purification. ¹H NMR (400 MHz, DMSO-d₆) δ 8.48 (dd, J=7.6, 0.8 Hz, 1H), 7.78 (t, J=0.8 Hz, 1H), 7.39 (d, J=1.2 Hz, 1H), 6.78 (d, J=8.8 Hz, 1H), 6.73 (dd, J=7.6, 2.4 Hz, 1H), 6.55-6.51 (m, 1H), 6.48 (dd, J=8.6, 2.8 Hz, 1H), 6.37-6.33 (m, 1H), 5.04 (brs, 2H), 1.99 (s, 3H). MS (ESI, m/z): 240 (M+H)⁺.

### Step 5. Synthesis of 6-bromo-N-(4-[imidazo[1,2-a]pyridin-7-yloxy]-3-methylphenyl)quinazolin-4-amine

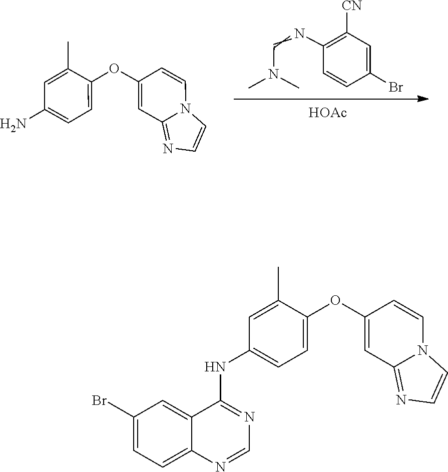 [SMILES: Cc1cc(N)ccc1Oc1ccn2ccnc2c1 ; CN(C)C=Nc1ccc(Br)cc1C#N ; Cc1cc(Nc2ncnc3ccc(Br)cc23)ccc1Oc1ccn2ccnc2c1 ; "HOAc"]

To a solution of 4-[imidazo[1,2-a]pyridin-7-yloxy]-3-methylaniline (180.0 mg, 0.75 mmol) in HOAc (5.0 mL) was added N′-(4-bromo-2-cyanophenyl)-N,N-dimethylformimidamide (189.7 mg, 0.75 mmol) at room temperature. The resulting mixture was stirred at 85° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (87/13, v/v) to afford 6-bromo-N-(4-[imidazo[1,2-a]pyridin-7-yloxy]-3-methylphenyl)quinazolin-4-amine (130.0 mg, 38%) as a yellow solid. LCMS (ESI, m/z): [M+H]⁺=446.1.

### Step 6. Synthesis of 1-[4-[(4-[imidazo[1,2-a]pyridin-7-yloxy]-3-methylphenyl)amino]quinazolin-6-yl]-3-methylidenepyrrolidin-2-one (Compound 4)

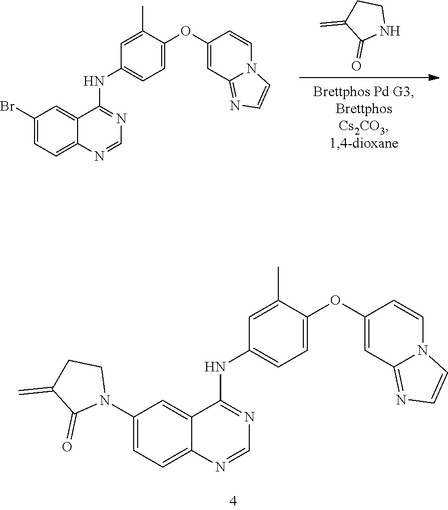

To a mixture of 6-bromo-N-(4-[imidazo[1,2-a]pyridin-7-yloxy]-3-methylphenyl)quinazolin-4-amine (100.0 mg, 0.22 mmol) and 3-methylidenepyrrolidin-2-one (65.3 mg, 0.67 mmol) in 1,4-dioxane (3.0 mL) was added Cs₂CO₃ (438.0 mg, 1.34 mmol), BrettPhos (24.0 mg, 0.04 mmol) and Brettphos Pd G3 (20.3 mg, 0.02 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (88/12, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 29% B to 59% B in 10 min; 254 nm) to afford 1-[4-[(4-[imidazo[1,2-a]pyridin-7-yloxy]-3-methylphenyl)amino]quinazolin-6-yl]-3-methylidenepyrrolidin-2-one (Compound 4) (11.6 mg, 11%) as a white solid. LCMS (ESI, m/z): [M+H]⁺=463.2. ¹H NMR (300 MHz, DMSO-d₆): δ 9.85 (s, 1H), 8.72-8.68 (m, 1H), 8.58-8.55 (m, 2H), 8.36 (d, J=2.1 Hz, 1H), 7.86-7.76 (m, 4H), 7.44 (d, J=0.9 Hz, 1H), 7.17 (d, J=8.1 Hz, 1H), 6.83-6.80 (m, 1H), 6.57 (d, J=2.4 Hz, 1H), 6.00 (s, 1H), 5.56 (s, 1H), 4.09-4.05 (m, 2H), 3.01-2.97 (m, 2H), 2.23 (s, 3H).

### Example S5: Synthesis of 1-methyl-4-(2-methyl-4-[[6-(3-methylidene-2-oxopyrrolidin-1-yl)quinazolin-4-yl]amino]phenoxy)pyridin-2-one (Compound 5)

### Step 1. Synthesis of 1-methyl-4-(2-methyl-4-nitrophenoxy)pyridin-2-one

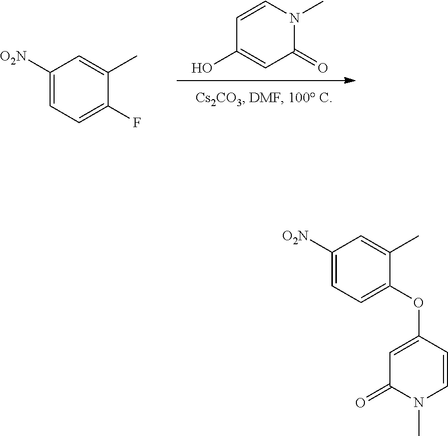

A mixture of 1-fluoro-2-methyl-4-nitrobenzene (1.0 g, 6.70 mmol), 4-hydroxy-1-methylpyridin-2-one (0.9 g, 7.11 mmol) and Cs₂CO₃ (4.4 g, 13.41 mmol) in DMF (20.0 mL) was stirred at 100 C for 4 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum to afford 1-methyl-4-(2-methyl-4-nitrophenoxy)pyridin-2-one (1.6 g, crude) as a yellow solid. LCMS (ESI, m/z): [M+H]⁺=261.0.

### Step 2. Synthesis of 4-(4-amino-2-methylphenoxy)-1-methylpyridin-2-one

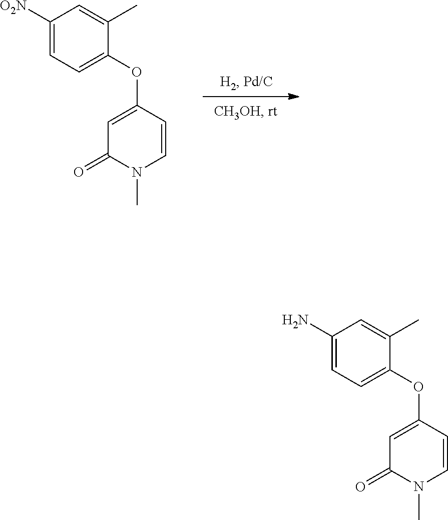 [SMILES: Cc1cc([N+](=O)[O-])ccc1Oc1ccn(C)c(=O)c1 ; Cc1cc(N)ccc1Oc1ccn(C)c(=O)c1]

A mixture of 1-methyl-4-(2-methyl-4-nitrophenoxy)pyridin-2-one (1.9 g, 5.99 mmol) and Pd/C (307.4 mg, dry) in CH₃OH (14.0 mL) was stirred at room temperature for 4 h under H₂. After the reaction was completed, the resulting mixture was filtered. The filtrate was concentrated under reduced pressure to afford 4-(4-amino-2-methylphenoxy)-1-methylpyridin-2-one (1.3 g, crude) as a brown yellow oil. LCMS (ESI, m/z): [M+H]⁺=231.1.

### Step 3. Synthesis of 4-(4-((6-bromoquinazolin-4-yl)amino)-2-methylphenoxy)-1-methylpyridin-2(1H)-one

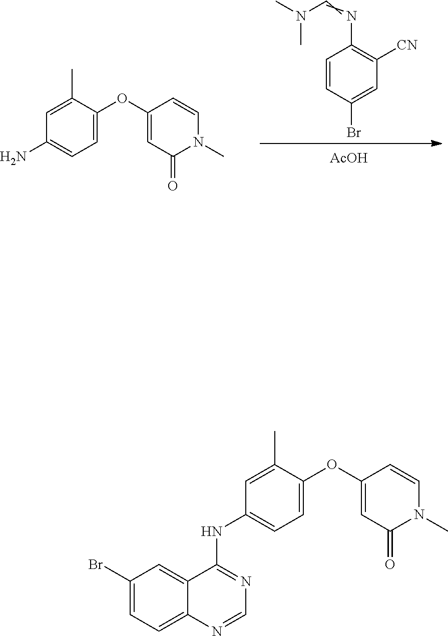 [SMILES: "AcOH" ; Cc1cc(N)ccc1Oc1ccn(C)c(=O)c1 ; CN(C)C=Nc1ccc(Br)cc1C#N ; Cc1cc(Nc2ncnc3ccc(Br)cc23)ccc1Oc1ccn(C)c(=O)c1]

A mixture of 4-(4-amino-2-methylphenoxy)-1-methylpyridin-2-one (900.0 mg, 3.90 mmol) and N′-(4-bromo-2-cyanophenyl)-N,N-dimethylformimidamide (985.3 mg, 3.90 mmol) in HOAc (20.0 mL) was stirred at 85° C. for 2 h. After the reaction was completed, the resulting mixture was concentrated under vacuum. The residue was purified by flash column chromatography with CH₂Cl₂/MeOH (90/10, v/v) to afford 4-(4-((6-bromoquinazolin-4-yl)amino)-2-methylphenoxy)-1-methylpyridin-2(1H)-one (1.5 g, 91%) as an off-white solid. LCMS (ESI, m/z): [M+H]⁺=437.1.

### Step 4. Synthesis of 1-methyl-4-(2-methyl-4-[[6-(3-methylidene-2-oxopyrrolidin-1-yl)quinazolin-4-yl]amino]phenoxy)pyridin-2-one (Compound 5)

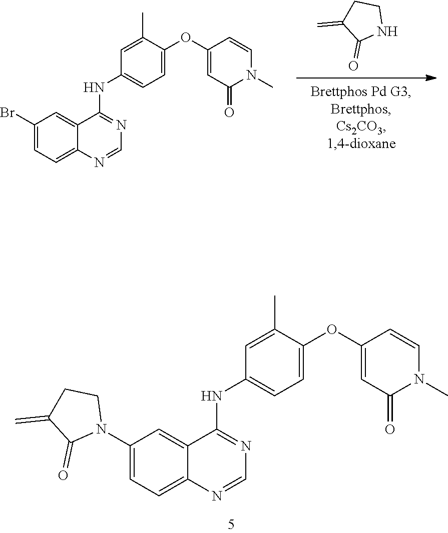

To a solution of 4-[4-[(6-bromoquinazolin-4-yl)amino]-2-methylphenoxy]-1-methylpyridin-2-one (400.0 mg, 0.91 mmol) in dioxane (10.0 mL) was added 3-methylidenepyrrolidin-2-one (106.6 mg, 1.09 mmol), Cs₂CO₃ (1.7 g, 5.48 mmol), BrettPhos (98.2 mg, 0.18 mmol) and Brettphos Pd G3 (82.9 mg, 0.09 mmol) at room temperature under N₂. The resulting mixture was stirred at 100° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (50/50, v/v) and then purified by Prep-HPLC with the following conditions (Column: Xselect CSH OBD Column 30×<150 mm, 5 um; Mobile Phase A: Water (0.1% FA), Mobile Phase B: ACN; Flow rate: 60 mL/min; Gradient: 9% B to 29% B in 7 min; 254/220 nm) to afford 1-methyl-4-(2-methyl-4-[[6-(3-methylidene-2-oxopyrrolidin-1-yl)quinazolin-4-yl]amino]phenoxy)pyridin-2-one (Compound 5) (46.2 mg, 11%) as a white solid. LCMS (ESI, m/z): [M+H]⁺=454.2. ¹H NMR (400 MHz, DMSO-d₆): δ 9.86 (s, 1H), 8.71 (d, J=2.0 Hz, 1H), 8.69 (d, J=2.4 Hz, 1H), 8.57 (s, 1H), 7.84 (d, J=9.2 Hz, 1H), 7.78-7.72 (m, 3H), 7.15 (d, J=9.6 Hz, 1H), 6.12-6.09 (m, 1H), 5.99 (s, 1H), 5.55 (s, 1H), 5.34 (d, J=2.4 Hz, 1H), 4.08-4.04 (m, 2H), 3.37 (s, 3H), 3.00-2.96 (m, 2H), 2.16 (s, 3H).

### Example S6: Synthesis of 1-[4-([3-methyl-4 [(5-methylpyrazin-2-yl)oxy]phenyl]amino)quinazolin-6-yl]-3-methylidenepyrrolidin-2-one (Compound 6)

### Step 1. Synthesis of 2-methyl-5-(2-methyl-4-nitrophenoxy)pyrazine

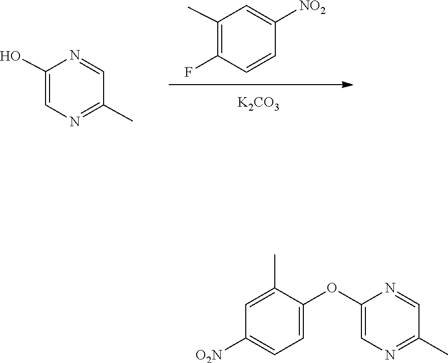

To a solution of 5-methylpyrazin-2-ol (5.0 g, 45.40 mmol) in DMF (50.0 mL) was added K₂CO₃ (12.5 g, 90.81 mmol) and 1-fluoro-2-methyl-4-nitrobenzene (7.0 g, 45.40 mmol). The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 2-methyl-5-(2-methyl-4-nitrophenoxy)pyrazine (5.1 g, 45%) as a light brown solid. LCMS (ESI, m/z): [M+H]⁺=246.0.

### Step 2. Synthesis of 3-methyl-4-[(5-methylpyrazin-2-yl)oxy]aniline

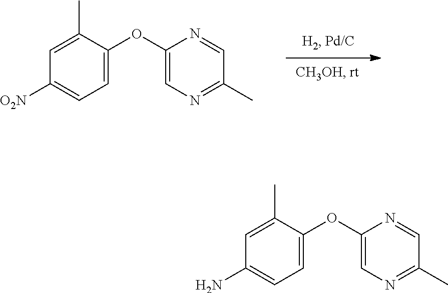 [SMILES: Cc1cnc(Oc2ccc([N+](=O)[O-])cc2C)cn1 ; Cc1cnc(Oc2ccc(N)cc2C)cn1]

To a solution of 2-methyl-5-(2-methyl-4-nitrophenoxy)pyrazine (5.1 g, 20.79 mmol) in MeOH (50.0 mL) was added Pd/C (1.7 g, dry). The resulting mixture was stirred at room temperature for 3 h under H₂. After the reaction was completed, the resulting mixture was filtered. The filtrate was concentrated under vacuum to afford 3-methyl-4-[(5-methylpyrazin-2-yl)oxy]aniline (4.4 g, crude) as a brown oil. LCMS (ESI, m/z): [M+H]⁺=216.0.

### Step 3. Synthesis of 6-bromo-N-[3-methyl-4-[(5-methylpyrazin-2-yl)oxy]phenyl]quinazolin-4-amine

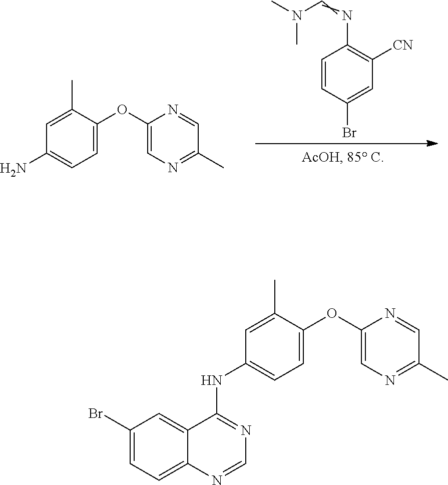 [SMILES: Cc1cnc(Oc2ccc(N)cc2C)cn1 ; CN(C)C=Nc1ccc(Br)cc1C#N ; "AcOH, 85° C." ; Cc1cnc(Oc2ccc(Nc3ncnc4ccc(Br)cc34)cc2C)cn1]

A mixture of 3-methyl-4-[(5-methylpyrazin-2-yl)oxy]aniline (500.0 mg, 2.32 mmol) and N′-(4-bromo-2-cyanophenyl)-N,N-dimethylformimidamide (585.6 mg, 2.32 mmol) in HOAc (10.0 mL) was stirred at 85° C. for 2 h. After the reaction was completed, the resulting mixture was concentrated under vacuum. The residue was purified by flash column chromatography with petroleum ether/ethyl acetate (1/1, v/v) to afford 6-bromo-N-[3-methyl-4-[(5-methylpyrazin-2-yl)oxy]phenyl]quinazolin-4-amine (760.0 mg, 77%) as a brown solid. LCMS (ESI, m/z): [M+H]⁺=422.3.

### Step 4. Synthesis of 1-[4-([3-methyl-4-[(5-methylpyrazin-2-yl)oxy]phenyl]amino)quinazolin-6-yl]-3-methylidenepyrrolidin-2-one (Compound 6)

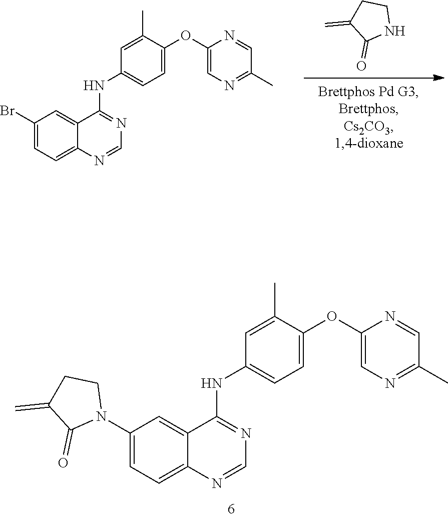

A mixture of 6-bromo-N-[3-methyl-4-[(5-methylpyrazin-2-yl)oxy]phenyl]quinazolin-4-amine (300.0 mg, 0.71 mmol), 3-methylidenepyrrolidin-2-one (206.9 mg, 2.13 mmol), Cs₂CO₃ (1388.8 mg, 4.26 mmol), BrettPhos (152.5 mg, 0.28 mmol) and Brettphos Pd G3 (128.8 mg, 0.14 mmol) in dioxane (8.0 mL) was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by reverse phase flash column chromatography with ACN in water (5% to 100% gradient in 40 min) and then purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 16% B to 30% B in 7 min; 254/220 nm) to afford 1-[4-([3-methyl-4-[(5-methylpyrazin-2-yl)oxy]phenyl]amino)quinazolin-6-yl]-3-methylidenepyrrolidin-2-one (Compound 6) (24.6 mg, 7%) as a light yellow solid. LCMS (ESI, m/z): [M+H]⁺=439.2. ¹H NMR (400 MHz, DMSO-d₆): δ 9.83 (s, 1H), 8.71-8.68 (m, 1H), 8.55 (s, 1H), 8.43 (d, J=1.2 Hz, 1H), 8.34 (d, J=2.0 Hz, 1H), 8.07 (s, 1H), 7.83 (d, J=9.2 Hz, 1H), 7.69-7.67 (m, 2H), 7.14 (d, J=8.4 Hz, 1H), 5.99-5.98 (m, 1H), 5.56 (s, 1H), 4.08-4.04 (m, 2H), 3.00-2.97 (m, 2H), 2.45 (m, 3H), 2.14 (s, 3H).

### Example S7: Synthesis of 1-[5-fluoro-4-[(3-methyl-4-[[1,2,4]triazolo[1,5-a]pyridin-7-yloxy]phenyl)amino]quinazolin-6-yl]-3-methylidenepyrrolidin-2-one (Compound 7)

### Step 1. Synthesis of 4-(2-methyl-4-nitrophenoxy)pyridin-2-amine

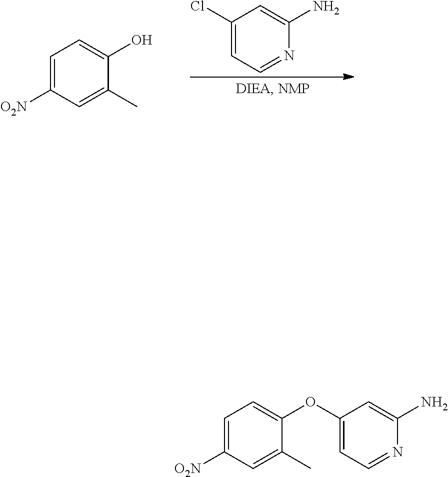 [SMILES: Cc1cc([N+](=O)[O-])ccc1O ; Nc1cc(Cl)ccn1 ; Cc1cc([N+](=O)[O-])ccc1Oc1ccnc(N)c1]

To a solution of 4-nitro-o-cresol (10.0 g, 65.30 mmol) in NMP (50.0 mL) was added 4-chloropyridin-2-amine (8.4 g, 65.30 mmol) and DIEA (16.9 g, 130.60 mmol) at room temperature. The resulting mixture was stirred at 150° C. for 24 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/8, v/v) to afford 4-(2-methyl-4-nitrophenoxy)pyridin-2-amine (2.9 g, 18%) as a brown solid. LCMS (ESI, m/z): [M+H]⁺=246.1.

### Step 2. Synthesis of (E)-N-hydroxy-N′-(4-(2-methyl-4-nitrophenoxy)pyridin-2-yl)formimidamide

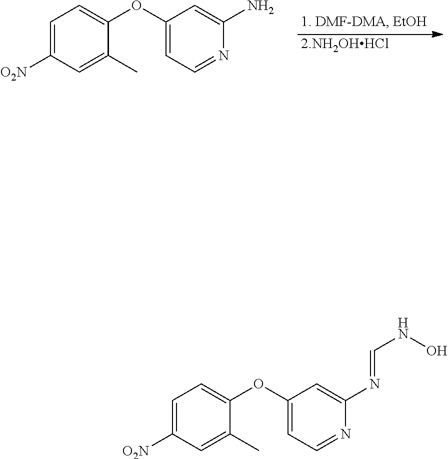 [SMILES: Cc1cc([N+](=O)[O-])ccc1Oc1ccnc(N)c1 ; "2.NH2OH·HCl" ; Cc1cc([N+](=O)[O-])ccc1Oc1ccnc(/N=C/NO)c1]

To a solution of 4-(2-methyl-4-nitrophenoxy)pyridin-2-amine (2.9 g, 11.83 mmol) in EtOH (30.0 mL) was added DMF-DMA (2.4 g, 20.10 mmol) at room temperature. The resulting mixture was stirred at 75° C. for 3 h. Then hydroxylamine hydrochloride (2.9 g, 41.39 mmol) was added to the mixture at 40° C. The resulting mixture was stirred at 50° C. for 0.5 h. After the reaction was completed, the reaction mixture was cooled to room temperature and then filtered. The solid was washed with EtOH and dried to afford (E)-N-hydroxy-N′-(4-(2-methyl-4-nitrophenoxy)pyridin-2-yl)formimidamide (2.7 g, crude) as a light brown solid. LCMS (ESI, m/z): [M+H]⁺=289.1.

### Step 3: Synthesis of 7-(2-methyl-4-nitrophenoxy)-[1,2,4]triazolo[1,5-a]pyridine (1c)

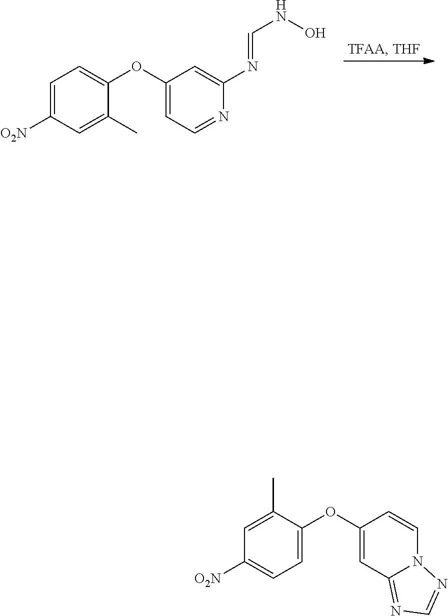 [SMILES: Cc1cc([N+](=O)[O-])ccc1Oc1ccnc(/N=C/NO)c1 ; Cc1cc([N+](=O)[O-])ccc1Oc1ccn2ncnc2c1]

To a solution of (E)-N-hydroxy-N′-(4-(2-methyl-4-nitrophenoxy)pyridin-2-yl)formimidamide (2.8 g, crude) in THF (40.0 mL) was added TFAA (8.0 g, 38.16 mmol) at 0° C. The resulting mixture was stirred at room temperature for 5 h. After the reaction was completed, the reaction was diluted with EtOAc and washed with NaHCO₃(aq), dried over anhydrous sodium sulfate and filtered. The filtrate was concentrated under reduced pressure. The residue was purified by flash column chromatography with petroleum ether/ethyl acetate (2/4, v/v) to afford 7-(2-methyl-4-nitrophenoxy)-[1,2,4]triazolo[1,5-a]pyridine (460.0 mg, 17%) as a white solid. LCMS (ESI, m/z): [M+H]⁺=271.1.

### Step 4. Synthesis of 3-methyl-4-[[1,2,4]triazolo[1,5-a]pyridin-7-yloxy]aniline

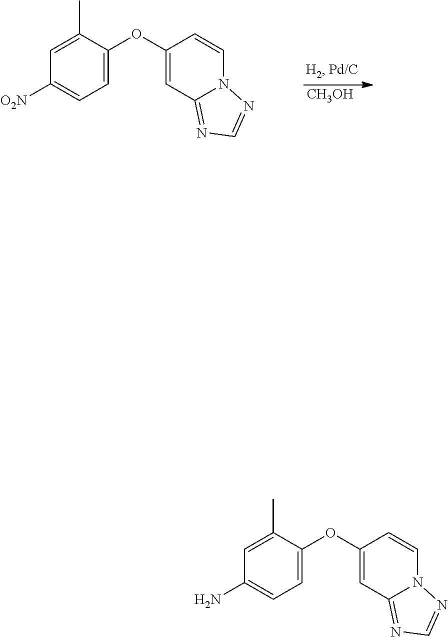 [SMILES: Cc1cc([N+](=O)[O-])ccc1Oc1ccn2ncnc2c1 ; "CH3OH" ; Cc1cc(N)ccc1Oc1ccn2ncnc2c1]

To a solution of 7-(2-methyl-4-nitrophenoxy)-[1,2,4]triazolo[1,5-a]pyridine (410.0 mg, 1.52 mmol) in MeOH (10.0 mL) was added Pd/C (120.0 mg, dry). The mixture was stirred at at room temperature for 3 h under H2. After the reaction was completed, the resulting mixture was filtered. The filtrate was concentrated under reduced pressure to afford 3-methyl-4-[[1,2,4]triazolo[1,5-a]pyridin-7-yloxy]aniline (360.0 mg, crude) as a yellow solid. LCMS (ESI, m/z): [M+H]⁺=241.1.

### Step 5. Synthesis of N′-(4-bromo-2-cyano-3-fluorophenyl)-N,N-dimethylformimidamide

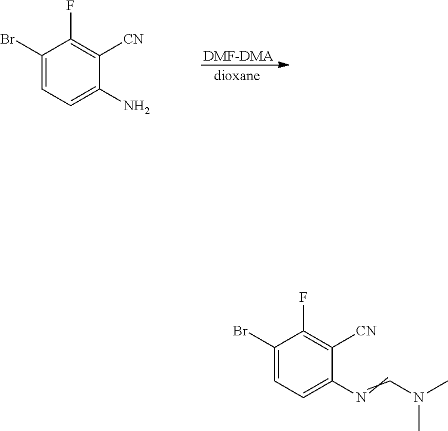

To a solution of 6-amino-3-bromo-2-fluorobenzonitrile (500.0 mg, 2.33 mmol) in 1,4-dioxane (5.0 mL) was added DMF-DMA (554.2 mg, 4.65 mmol) at room temperature. The resulting mixture was stirred at 75° C. for 30 min. After the reaction was completed, the resulting mixture was concentrated under reduced pressure to afford N′-(4-bromo-2-cyano-3-fluorophenyl)-N,N-dimethylformimidamide (600.0 mg, crude) as a yellow solid. LCMS (ESI, m/z): [M+H]⁺=270.0.

### Step 6. Synthesis of 6-bromo-5-fluoro-N-(3-methyl-4-[[1,2,4]triazolo[1,5-a]pyridin-7-yloxy]phenyl)quinazolin-4-amine

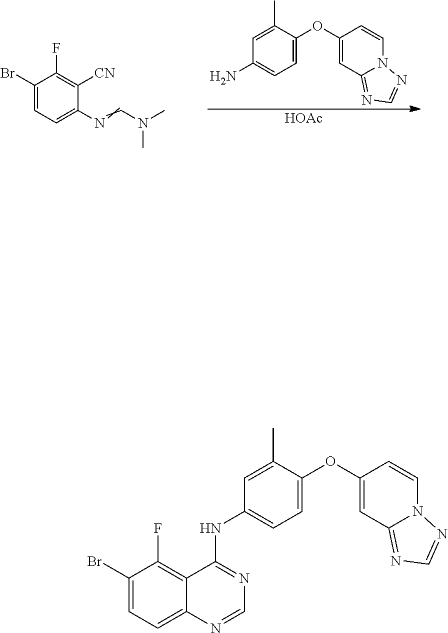 [SMILES: CN(C)C=Nc1ccc(Br)c(F)c1C#N ; Cc1cc(N)ccc1Oc1ccn2ncnc2c1 ; Cc1cc(Nc2ncnc3ccc(Br)c(F)c23)ccc1Oc1ccn2ncnc2c1 ; "HOAc"]

To a solution of N′-(4-bromo-2-cyano-3-fluorophenyl)-N,N-dimethylformimidamide (300.0 mg, 1.11 mmol) in acetic acid (10.0 mL) was added 3-methyl-4-[[1,2,4]triazolo[1,5-a]pyridin-7-yloxy]aniline (266.9 mg, 1.11 mmol) at room temperature. The resulting mixture was stirred at 85° C. for 2 h. After the reaction was completed, the resulting mixture was concentrated under reduced pressure. The residue was purified by flash column chromatography with CH₂Cl₂/CH₃OH (89/11, v/v) to afford 6-bromo-5-fluoro-N-(3-methyl-4-[[1,2,4]triazolo[1,5-a]pyridin-7-yloxy]phenyl)quinazolin-4-amine (270.0 mg, 52%) as a yellow solid. LCMS (ESI, m/z): [M+H]⁺=465.0.

### Step 7. Synthesis of 1-[5-fluoro-4-[(3-methyl-4-[[1,2,4]triazolo[1,5-a]pyridin-7-yloxy]phenyl)amino]quinazolin-6-yl]-3-methylidenepyrrolidin-2-one (Compound 7)

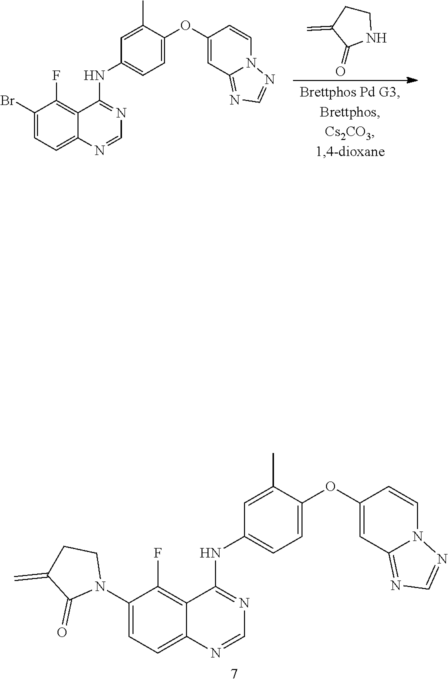

To a solution of 6-bromo-5-fluoro-N-(3-methyl-4-[[1,2,4]triazolo[1,5-a]pyridin-7-yloxy]phenyl)quinazolin-4-amine (200.0 mg, 0.43 mmol) in 1,4-dioxane (5.0 mL) was added 3-methylidenepyrrolidin-2-one (125.2 mg, 1.29 mmol), Cs₂CO₃ (840.3 mg, 2.58 mmol), BrettPhos (46.2 mg, 0.08 mmol) and Brettphos Pd G3 (38.9 mg, 0.04 mmol) at room temperature under N₂. The resulting mixture was stirred at 100° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (87/13, v/v) and then purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 14% B to 43% B in 7 min; 254 nm) to afford 1-[5-fluoro-4-[(3-methyl-4-[[1,2,4]triazolo[1,5-a]pyridin-7-yloxy]phenyl)amino]quinazolin-6-yl]-3-methylidenepyrrolidin-2-one (Compound 7) (16.5 mg, 8%) as a white solid. LCMS (ESI, m/z): [M+H]⁺=482.0. ¹H NMR (300 MHz, DMSO-d₆): 9.29-9.25 (m, 1H), 8.95 (d, J=7.2 Hz, 1H), 8.63 (s, 1H), 8.39 (s, 1H), 8.02-7.96 (m, 1H), 7.77-7.71 (m, 3H), 7.22 (d, J=8.4 Hz, 1H), 7.04 (d, J=5.7 Hz, 1H), 6.82 (s, 1H), 5.96 (s, 1H), 5.56 (s, 1H), 3.95-3.90 (m, 2H), 3.05-2.95 (m, 2H), 2.20 (s, 3H).

### Example S8: Synthesis of 3-methylidene-1-[4-([4-[(6-methylpyridin-3-yl)methyl]phenyl]amino)quinazolin-6-yl]pyrrolidin-2-one (Compound 8)

### Step 1. Synthesis of 2-methyl-5-[(4-nitrophenyl)methyl]pyridine

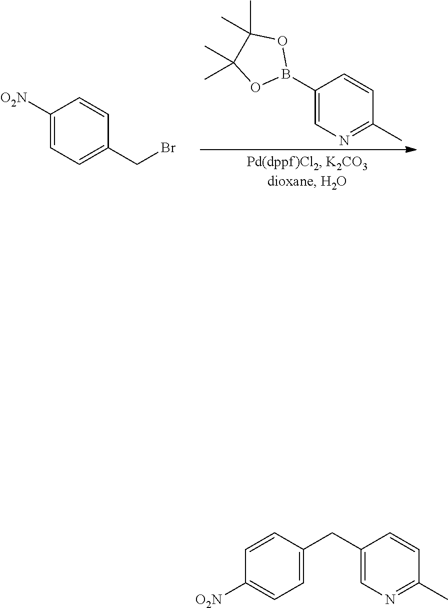

To a solution of nitrobenzyl bromide (2.0 g, 9.26 mmol) in 1,4-dioxane/H₂O (10.0/2.0 mL) was added 2-methyl-5-(4,4,5,5-tetramethyl-1,3,2-dioxaborolan-2-yl)pyridine (2.4 g, 11.11 mmol), K₂CO₃ (3.8 g, 27.77 mmol) and Pd(dppf)Cl₂ (677.4 mg, 0.93 mmol) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (9/1, v/v) to afford 2-methyl-5-[(4-nitrophenyl)methyl]pyridine (780.0 mg, 36%) as a yellow solid. LCMS (ESI, m/z): [M+H]⁺=229.1.

### Step 2. Synthesis of 4-[(6-methylpyridin-3-yl)methyl]aniline

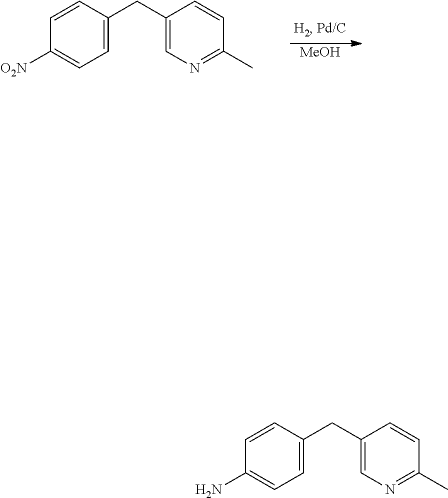 [SMILES: "MeOH" ; Cc1ccc(Cc2ccc([N+](=O)[O-])cc2)cn1 ; Cc1ccc(Cc2ccc(N)cc2)cn1]

To a solution of 2-methyl-5-[(4-nitrophenyl)methyl]pyridine (780.0 mg, 3.42 mmol) in CH₃OH (10.0 mL) was added Pd/C (363.7 mg, dry) at room temperature. The resulting mixture was stirred at room temperature for 2 h under H2. After the reaction was completed, the resulting mixture was filtered. The filtrate was concentrated under reduced pressure. The residue was purified by flash column chromatography with CH₂Cl₂/CH₃OH (95/5, v/v) to afford 4-[(6-methylpyridin-3-yl)methyl]aniline (300.0 mg, 44%) as a yellow solid. LCMS (ESI, m/z): [M+H]⁺=199.1.

### Step 3. Synthesis of 6-bromo-N-[4-[(6-methylpyridin-3-yl)methyl]phenyl]quinazolin-4-amine

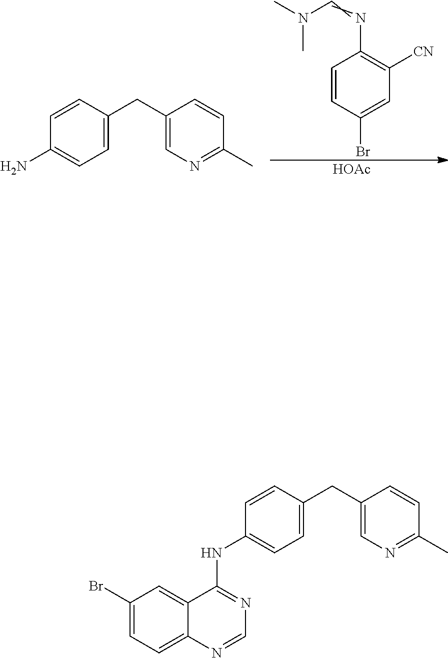 [SMILES: Cc1ccc(Cc2ccc(N)cc2)cn1 ; CN(C)C=Nc1ccc(Br)cc1C#N ; Cc1ccc(Cc2ccc(Nc3ncnc4ccc(Br)cc34)cc2)cn1 ; "HOAc"]

To a solution of 4-[(6-methylpyridin-3-yl)methyl]aniline (100.0 mg, 0.50 mmol) in AcOH (5.0 mL) was added N′-(4-bromo-2-cyanophenyl)-N,N-dimethylformimidamide (127.2 mg, 0.50 mmol) at room temperature. The resulting mixture was stirred at 85° C. for 2 h. After the reaction was completed, the resulting mixture was concentrated under reduced pressure. The residue was purified by flash column chromatography with CH₂Cl₂/CH₃OH (89/11, v/v) to afford 6-bromo-N-[4-[(6-methylpyridin-3-yl)methyl]phenyl]quinazolin-4-amine (200.0 mg, 97%) as a yellow solid. LCMS (ESI, m/z): [M+H]⁺=405.0.

### Step 4. Synthesis of 3-methylidene-1-[4-([4-[(6-methylpyridin-3-yl)methyl]phenyl]amino)quinazolin-6-yl]pyrrolidin-2-one (Compound 8)

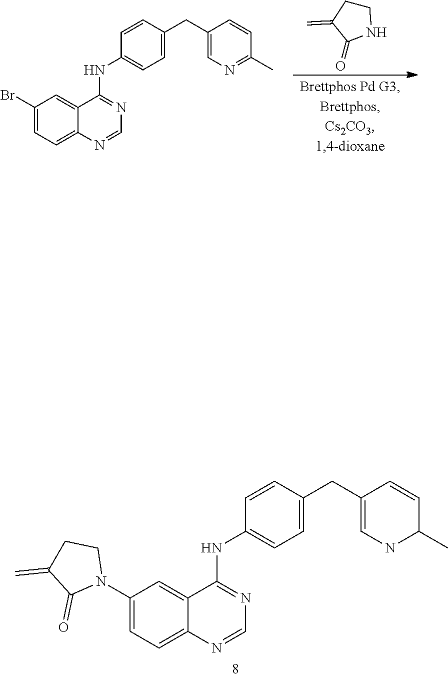

To a solution of 6-bromo-N-[4-[(6-methylpyridin-3-yl)methyl]phenyl]quinazolin-4-amine (150.0 mg, 0.37 mmol) in 1,4-dioxane (5.0 mL) was added 3-methylidenepyrrolidin-2-one (107.8 mg, 1.11 mmol), Cs₂CO₃ (723.5 mg, 2.22 mmol), BrettPhos (39.7 mg, 0.07 mmol) and Brettphos Pd G3 (33.6 mg, 0.04 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (92/8, v/v) and then purified by Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 42% B to 51% B in 8 min; 254 nm) to afford 3-methylidene-1-[4-([4-[(6-methylpyridin-3-yl)methyl]phenyl]amino)quinazolin-6-yl]pyrrolidin-2-one (Compound 8) (34.5 mg, 22%) as a white solid. LCMS (ESI, m/z): [M+H]⁺=422.2. ¹H NMR (300 MHz, DMSO-d₆): δ 9.80 (s, 1H), 8.70-8.66 (m, 1H), 8.51 (s, 1H), 8.40-8.32 (m, 2H), 7.82 (d, J=9.0 Hz, 1H), 7.70 (d, J=8.4 Hz, 2H), 7.56-7.52 (m, 1H), 7.28 (d, J=8.7 Hz, 2H), 7.19 (d, J=7.8 Hz, 1H), 5.99-5.97 (m, 1H), 5.55 (s, 1H), 4.07-4.02 (m, 2H), 3.94 (s, 2H), 2.99-2.95 (m, 2H), 2.43 (s, 3H).

### Example S9: Synthesis of 3-methylidene-1-[4-[(4-[[1,2,4]triazolo[1,5-a]pyridin-7-ylmethyl]phenyl)amino]quinazolin-6-yl]pyrrolidin-2-one (Compound 9)

### Step 1. Synthesis of 7-(4,4,5,5-tetramethyl-1,3,2-dioxaborolan-2-yl)-[1,2,4]triazolo[1,5-a]pyridine

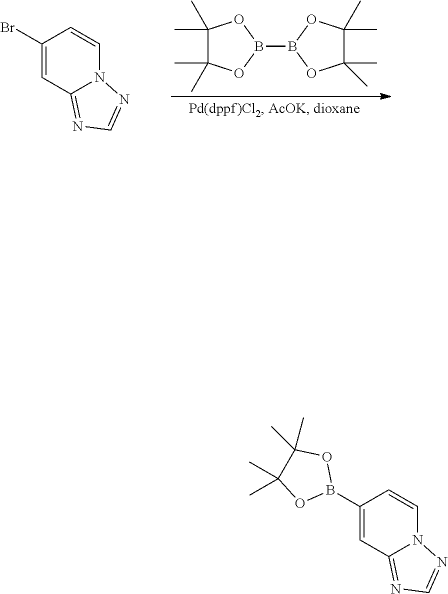

To a solution of 7-bromo-[1,2,4]triazolo[1,5-a]pyridine (1.0 g, 5.05 mmol) in 1,4-dioxane (10.0 mL) was added bis(pinacolato)diboron (3.8 g, 15.15 mmol), AcOK (1.5 g, 15.15 mmol) and Pd(dppf)Cl₂ (369.5 mg, 0.51 mmol) at room temperature under N₂. The resulting mixture was stirred at 80° C. for 16 h under N₂. After the reaction was completed, the resulting mixture was diluted with H₂O and extracted with ethyl acetate. The aqueous layer was concentrated under reduced pressure to afford 7-(4,4,5,5-tetramethyl-1,3,2-dioxaborolan-2-yl)-[1,2,4]triazolo[1,5-a]pyridine (1.0 g, crude) as a white solid. LCMS (ESI, m/z): [M+H]⁺=246.1

### Step 2. Synthesis of 7-[(4-nitrophenyl)methyl]-[1,2,4]triazolo[1,5-a]pyridine

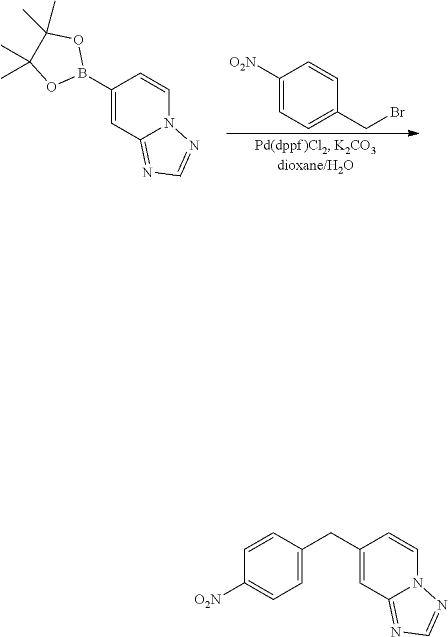 [SMILES: CC1(C)OB(c2ccn3ncnc3c2)OC1(C)C ; O=[N+]([O-])c1ccc(CBr)cc1 ; O=[N+]([O-])c1ccc(Cc2ccn3ncnc3c2)cc1]

To a solution of 1-(bromomethyl)-4-nitrobenzene (1.0 g, 4.63 mmol) in 1,4-dioxane/H₂O (10.0/2.0 mL) was added K₂CO₃ (1.9 g, 13.89 mmol), 7-(4,4,5,5-tetramethyl-1,3,2-dioxaborolan-2-yl)-[1,2,4]triazolo[1,5-a]pyridine (3.4 g, 13.89 mmol) and Pd(dppf)Cl₂ (338.7 mg, 0.46 mmol) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (47/53, v/v) to afford 7-[(4-nitrophenyl)methyl]-[1,2,4]triazolo[1,5-a]pyridine (500.0 mg, 42%) as a white solid. LCMS (ESI, m/z): [M+H]⁺=255.0.

### Step 3. Synthesis of 4-[[1,2,4]triazolo[1,5-a]pyridin-7-ylmethyl]aniline

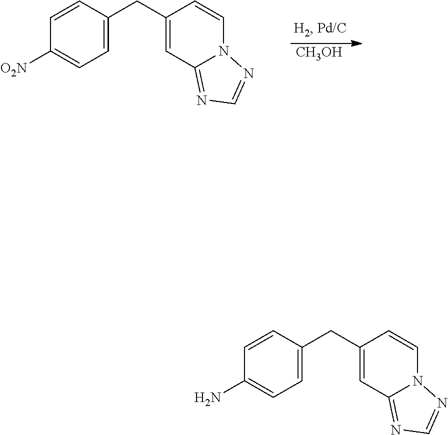

To a solution of 7-[(4-nitrophenyl)methyl]-[1,2,4]triazolo[1,5-a]pyridine (450.0 mg, 1.77 mmol) in MeOH (10.0 mL) was added Pd/C (188.4 mg, dry) at room temperature under N₂. The resulting mixture was stirred at room temperature for 2 h under H2. After the reaction was completed, the resulting mixture was filtered. The filtrate was concentrated under reduced pressure. The residue was purified by flash column chromatography with CH₂Cl₂/CH₃OH (9/1, v/v) to afford 4-[[1,2,4]triazolo[1,5-a]pyridin-7-ylmethyl]aniline (300.0 mg, 75%) as a yellow solid. LCMS (ESI, m/z): [M+H]⁺=225.1.

### Step 4. Synthesis of 6-bromo-N-(4-[[1,2,4]triazolo[1,5-a]pyridin-7-ylmethyl]phenyl)quinazolin-4-amine

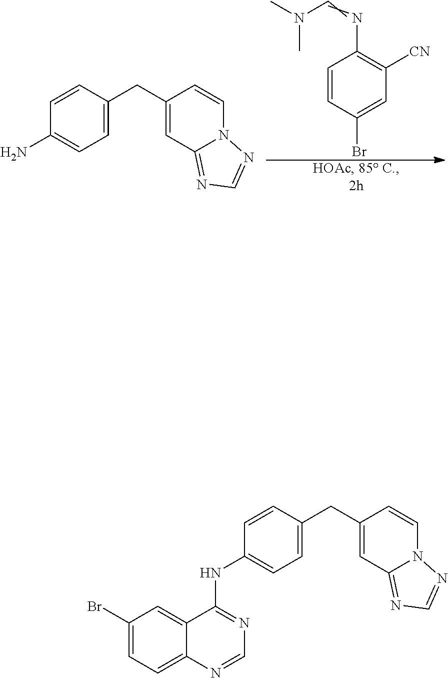

To a solution of 4-[[1,2,4]triazolo[1,5-a]pyridin-7-ylmethyl]aniline (150.0 mg, 0.66 mmol) in HOAc (5.0 mL) was added N′-(4-bromo-2-cyanophenyl)-N,N-dimethylformimidamide (168.6 mg, 0.66 mmol) at room temperature. The resulting mixture was stirred at 85° C. for 2 h. After the reaction was completed, the resulting mixture was concentrated under vacuum. The residue was purified by flash column chromatography with dichloromethane/methanol (10/1, v/v) to afford 6-bromo-N-(4-[[1,2,4]triazolo[1,5-a]pyridin-7-ylmethyl]phenyl)quinazolin-4-amine (200.0 mg, 69%) as a brown solid. LCMS (ESI, m/z): [M+H]⁺=431.0.

### Step 5. Synthesis of 3-methylidene-1-[4-[(4-[[1,2,4]triazolo[1,5-a]pyridin-7-ylmethyl]phenyl)amino]quinazolin-6-yl]pyrrolidin-2-one (Compound 9)

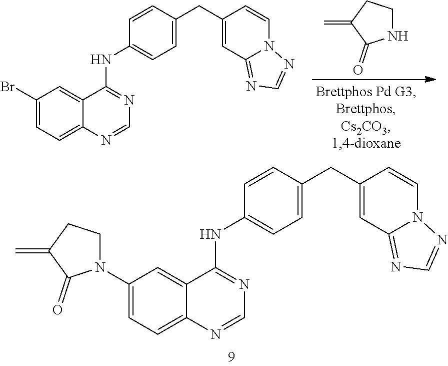

To a solution of 6-bromo-N-(4-[[1,2,4]triazolo[1,5-a]pyridin-7-ylmethyl]phenyl)quinazolin-4-amine (150.0 mg, 0.35 mmol) in 1,4-dioxane (9.0 mL) was added 3-methylidenepyrrolidin-2-one (101.3 mg, 1.04 mmol), Cs₂CO₃ (226.6 mg, 0.70 mmol), BrettPhos (37.3 mg, 0.07 mmol) and Brettphos Pd G3 (31.5 mg, 0.04 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: Column: Xselect CSH OBD Column 30×150 mm, 5 um; Mobile Phase A: Water (0.1% FA), Mobile Phase B: ACN; Flow rate: 60 mL/min; Gradient: 9% B to 31% B in 7 min; 254/220 nm; RT1:7 min to afford 3-methylidene-1-[4-[(4-[[1,2,4]triazolo[1,5-a]pyridin-7-ylmethyl]phenyl)amino]quinazolin-6-yl]pyrrolidin-2-one (Compound 9) (27.6 mg, 17%) as a light yellow solid. LCMS (ESI, m/z): [M+H]⁺=448.2. ¹H NMR (400 MHz, DMSO-d₆): δ 9.83 (s, 1H), 8.86 (d, J=6.8 Hz, 1H), 8.69-8.66 (m, 1H), 8.52 (s, 1H), 8.44 (s, 1H), 8.33 (d, J=2.0 Hz, 1H), 7.82 (d, J=9.2 Hz, 1H), 7.75-7.72 (m, 3H), 7.37 (d, J=8.4 Hz, 2H), 7.10-7.08 (m, 1H), 5.97 (d, J=2.8 Hz, 1H), 5.54 (s, 1H), 4.12 (s, 2H), 4.06-4.02 (m, 2H), 2.99-2.95 (m, 2H).

### Example S10: Synthesis of 1-[4-[(3-chloro-4-[[1,2,4]triazolo[1,5-a]pyridin-7-yloxy]phenyl)amino]quinazolin-6-yl]-3-methylidenepyrrolidin-2-one (Compound 10)

### Step 1. Synthesis of 7-(2-chloro-4-nitrophenoxy)-[1,2,4]triazolo[1,5-a]pyridine

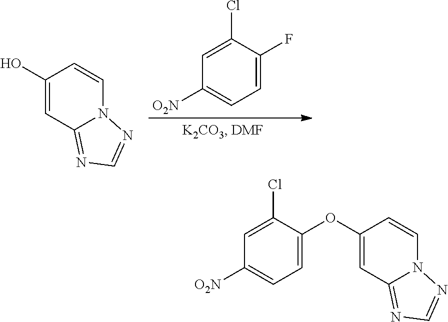 [SMILES: Oc1ccn2ncnc2c1 ; O=[N+]([O-])c1ccc(F)c(Cl)c1 ; O=[N+]([O-])c1ccc(Oc2ccn3ncnc3c2)c(Cl)c1]

A mixture of 2-chloro-1-fluoro-4-nitrobenzene (2.0 g, 11.39 mmol), [1,2,4]triazolo[1,5-a]pyridin-7-ol (1.9 g, 13.67 mmol) and K₂CO₃ (3.2 g, 22.79 mmol) in DMF (30.0 mL) was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (26/74, v/v) to afford 7-(2-chloro-4-nitrophenoxy)-[1,2,4]triazolo[1,5-a]pyridine (3.1 g, 93%) as a yellow solid. LCMS (ESI, m/z): [M+H]⁺=291.0.

### Step 2. Synthesis of 3-chloro-4-[[1,2,4]triazolo[1,5-a]pyridin-7-yloxy]aniline

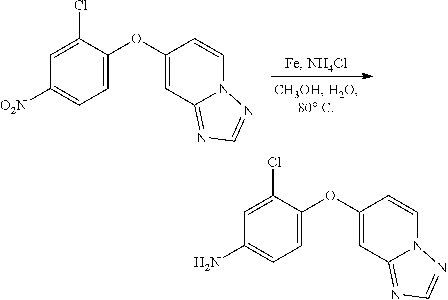

A solution of 7-(2-chloro-4-nitrophenoxy)-[1,2,4]triazolo[1,5-a]pyridine (3.0 g, 10.32 mmol), Fe (5.8 g, 103.21 mmol) and NH₄Cl (5521.0 mg, 103.21 mmol) in CH₃OH (40.0 mL) and H₂O (4.0 ml) was stirred at 80° C. for 1 h. After the reaction was completed, the resulting mixture was filtered. The filtrate was concentrated under reduced pressure. The residue was purified by flash column chromatography with CH₂Cl₂/CH₃OH (94/6, v/v) to afford 3-chloro-4-[[1,2,4]triazolo[1,5-a]pyridin-7-yloxy]aniline (2.3 g, 85%) as a yellow solid. LCMS (ESI, m/z): [M+H]⁺=261.0.

### Step 3. Synthesis of 6-bromo-N-(3-chloro-4-[[1,2,4]triazolo[1,5-a]pyridin-7-yloxy]phenyl)quinazolin-4-amine

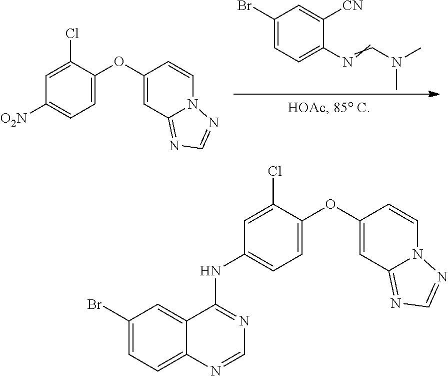 [SMILES: O=[N+]([O-])c1ccc(Oc2ccn3ncnc3c2)c(Cl)c1 ; CN(C)C=Nc1ccc(Br)cc1C#N ; Clc1cc(Nc2ncnc3ccc(Br)cc23)ccc1Oc1ccn2ncnc2c1]

A solution of 3-chloro-4-[[1,2,4]triazolo[1,5-a]pyridin-7-yloxy]aniline (500.0 mg, 1.92 mmol) and N′-(4-bromo-2-cyanophenyl)-N,N-dimethylformimidamide (580.3 mg, 2.30 mmol) in HOAc (15.0 mL) was stirred at 85° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford 6-bromo-N-(3-chloro-4-[[1,2,4]triazolo[1,5-a]pyridin-7-yloxy]phenyl)quinazolin-4-amine (750.0 mg, 83%) as a yellow solid. LCMS (ESI, m/z): [M+H]⁺=467.0.

### Step 4. Synthesis of 1-[4-[(3-chloro-4-[[1,2,4]triazolo[1,5-a]pyridin-7-yloxy]phenyl)amino]quinazolin-6-yl]-3-methylidenepyrrolidin-2-one (Compound 10)

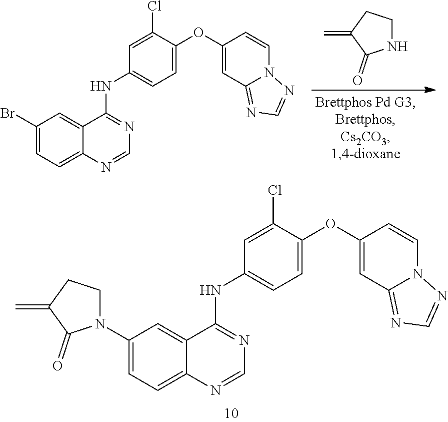

To a solution of 6-bromo-N-(3-chloro-4-[[1,2,4]triazolo[1,5-a]pyridin-7-yloxy]phenyl)quinazolin-4-amine (300.0 mg, 0.64 mmol) in dioxane (15.0 mL) was added 3-methylidenepyrrolidin-2-one (186.9 mg, 1.92 mmol), BrettPhos (68.9 mg, 0.13 mmol), BrettPhos Pd G3 (58.2 mg, 0.06 mmol) and Cs₂CO₃ (627.0 mg, 1.92 mmol) at room temperature. The mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) and then purified by Prep-HPLC with the following conditions (Column: XBridge Prep OBD C18 Column, 30×150 mm 5 um; Mobile Phase A: Water (10 mmol/L NH₄HCO₃), Mobile Phase B: ACN; Flow rate: 60 mL/min; Gradient: 23% B to 53% B in 9 min; 254 nm) to afford 1-[4-[(3-chloro-4-[[1,2,4]triazolo[1,5-a]pyridin-7-yloxy]phenyl)amino]quinazolin-6-yl]-3-methylidenepyrrolidin-2-one (Compound 10) (35.0 mg, 11%) as a white solid. LCMS (ESI, m/z): [M+H]⁺=484.1. ¹H NMR (400 MHz, DMSO-d₆) δ 10.00 (s, 1H), 8.97 (d, J=7.2 Hz, 1H), 8.71-8.66 (m, 2H), 8.42-8.39 (m, 2H), 8.28 (d, J=2.8 Hz, 1H), 7.98-7.96 (m, 1H), 7.88 (d, J=9.2 Hz, 1H), 7.50 (d, J=9.2 Hz, 1H), 7.10-7.07 (m, 1H), 6.96 (d, J=2.4 Hz, 1H), 6.00-5.99 (m, 1H), 5.56 (s, 1H), 4.08-4.05 (m, 2H), 3.01-2.97 (m, 2H).

### Example S11: Synthesis of 1-[4-[(3 fluoro-4-[[1,2,4]triazolo[1,5-a]pyridin-7-yloxy]phenyl)amino]quinazolin-6-yl]-3-methylidenepyrrolidin-2-one (Compound 11)

### Step 1. Synthesis of 7-(2-fluoro-4-nitrophenoxy)-[1,2,4]triazolo[1,5-a]pyridine

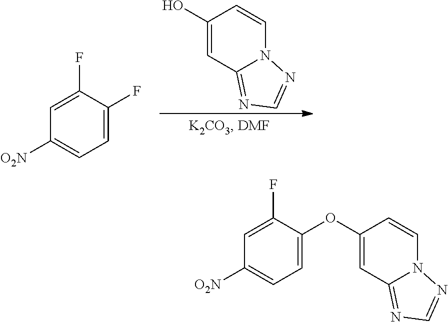 [SMILES: O=[N+]([O-])c1ccc(F)c(F)c1 ; Oc1ccn2ncnc2c1 ; O=[N+]([O-])c1ccc(Oc2ccn3ncnc3c2)c(F)c1]

To a mixture of 1,2-difluoro-4-nitrobenzene (2.0 g, 12.57 mmol) and [1,2,4]triazolo[1,5-a]pyridin-7-ol (1.7 g, 12.57 mmol) in DMF (30.0 mL) was added K₂CO₃ (3.5 g, 25.14 mmol) at room temperature. The resulting mixture was stirred at 70° C. for 2.5 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/7, v/v) to afford 7-(2-fluoro-4-nitrophenoxy)-[1,2,4]triazolo[1,5-a]pyridine (2.5 g, 72%) as a white solid. LCMS (ESI, m/z): [M+H]⁺=275.0.

### Step 2. Synthesis of 3-fluoro-4-[[1,2,4]triazolo[1,5-a]pyridin-7-yloxy]aniline

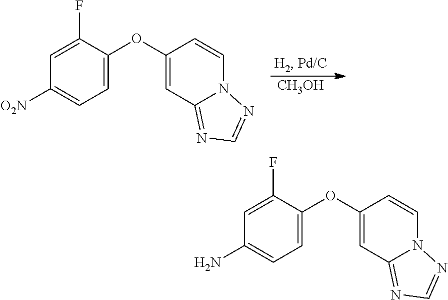

To a solution of 7-(2-fluoro-4-nitrophenoxy)-[1,2,4]triazolo[1,5-a]pyridine (2.6 g, 9.77 mmol) in MeOH (25.0 mL) was added Pd/C (800.0 mg, dry). The resulting mixture was stirred at room temperature for 3 h under H2. After the reaction was completed, the resulting mixture was filtered. The filtrate was concentrated under vacuum to afford 3-fluoro-4-[[1,2,4]triazolo[1,5-a]pyridin-7-yloxy]aniline (1.3 g, crude) as a yellow solid. LCMS (ESI, m/z): [M+H]⁺=245.0.

### Step 3. Synthesis of 6-bromo-N-(3-fluoro-4-[[1,2,4]triazolo[1,5-a]pyridin-7-yloxy]phenyl)quinazolin-4-amine

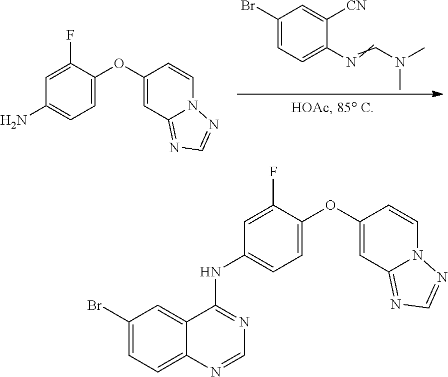 [SMILES: Nc1ccc(Oc2ccn3ncnc3c2)c(F)c1 ; CN(C)C=Nc1ccc(Br)cc1C#N ; Fc1cc(Nc2ncnc3ccc(Br)cc23)ccc1Oc1ccn2ncnc2c1]

A mixture of 3-fluoro-4-[[1,2,4]triazolo[1,5-a]pyridin-7-yloxy]aniline (400.0 mg, 1.63 mmol) and N′-(4-bromo-2-cyanophenyl)-N,N-dimethylformimidamide (412.9 mg, 1.63 mmol) in HOAc (8.0 mL) was stirred at 85° C. for 2 h. After the reaction was completed, the resulting mixture was concentrated under reduced pressure. The residue was purified by flash column chromatography with CH₂Cl₂/MeOH (10/1, v/v) to afford 6-bromo-N-(3-fluoro-4-[[1,2,4]triazolo[1,5-a]pyridin-7-yloxy]phenyl)quinazolin-4-amine (420.0 mg, 56%) as an off-white solid. LCMS (ESI, m/z): [M+H]⁺=451.0.

### Step 4. Synthesis of 1-[4-[(3-fluoro-4-[[1,2,4]triazolo[1,5-a]pyridin-7-yloxy]phenyl)amino]quinazolin-6-yl]-3-methylidenepyrrolidin-2-one (Compound 11)

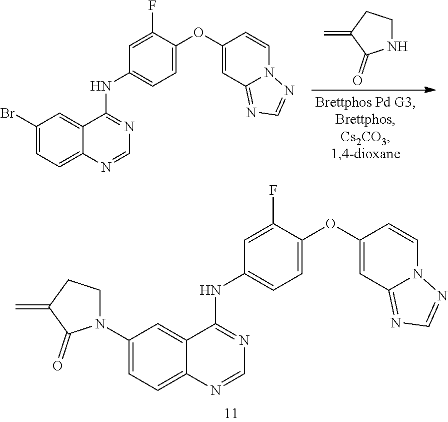

To a solution of 6-bromo-N-(3-fluoro-4-[[1,2,4]triazolo[1,5-a]pyridin-7-yloxy]phenyl)quinazolin-4-amine (200.0 mg, 0.44 mmol) in dioxane (5.0 mL) was added 3-methylidenepyrrolidin-2-one (129.1 mg, 1.33 mmol), Cs₂CO₃ (866.4 mg, 2.65 mmol), BrettPhos (95.1 mg, 0.17 mmol) and Brettphos Pd G3 (80.3 mg, 0.08 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) and then purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 36% B to 45% B in 8 min; 254/220 nm) to afford 1-[4-[(3-fluoro-4-[[1,2,4]triazolo[1,5-a]pyridin-7-yloxy]phenyl)amino]quinazolin-6-yl]-3-methylidenepyrrolidin-2-one (Compound 11) (13.3 mg, 6%) as a white solid. LCMS (ESI, m/z): [M+H]⁺=468.2. ¹H NMR (300 MHz, DMSO-d₆): δ 10.01 (s, 1H), 8.96 (d, J=7.5 Hz, 1H), 8.69-8.65 (m, 2H), 8.64-8.15 (m, 2H), 8.19-8.13 (m, 1H), 7.86 (d, J=9.3 Hz, 1H), 7.72-7.70 (m, 1H), 7.51-7.45 (m, 1H), 7.72-7.70 (m, 1H), 7.11-7.04 (m, 1H), 7.03 (s, 1H), 5.99-5.97 (m, 1H), 5.55 (s, 1H), 4.08-4.03 (m, 1H), 2.99-2.95 (m, 2H).

### Example S12: Synthesis of 1-(difluoromethyl)-4-(2-methyl-4-[[6-(3-methylidene-2-oxopyrrolidin-1 yl)quinazolin-4 yl]amino]phenoxy)pyridin-2-one (Compound 12)

### Step 1a. Synthesis of 2-chloro-4-(2-methyl-4-nitrophenoxy)pyridine

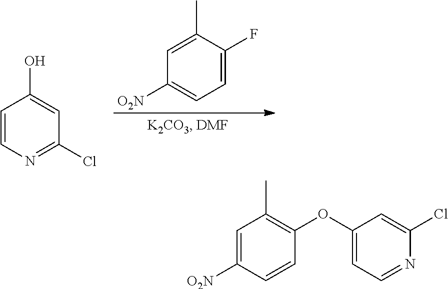 [SMILES: Oc1ccnc(Cl)c1 ; Cc1cc([N+](=O)[O-])ccc1F ; Cc1cc([N+](=O)[O-])ccc1Oc1ccnc(Cl)c1]

A mixture of 2-chloropyridin-4-ol (5.0 g, 38.59 mmol), 1-fluoro-2-methyl-4-nitrobenzene (7.1 g, 46.31 mmol) and K₂CO₃ (10.6 g, 77.19 mmol) in DMF (50.0 mL) was stirred at 50° C. for 16 h. After the reaction was completed, the resulting mixture was filtered. The filtrate was concentrated under vacuum. The residue was purified by reverse phase flash column chromatography with ACN in water (5% to 100% gradient in 40 min) to afford 2-chloro-4-(2-methyl-4-nitrophenoxy)pyridine (2.9 g, 28%) as a yellow solid. LCMS (ESI, m/z): [M+H]⁺=265.0.

### Step 1b: Alternative Synthesis of 2-chloro-4-(2-methyl-4-nitrophenoxy)pyridine

In an alternative to Step 1a: A mixture of 2-chloropyridin-4-ol (25.0 g), 1-fluoro-2-methyl-4-nitrobenzene (33.0 g) and K₂CO₃ (53.5 g) in DMF (75.0 mL) was stirred at 80° C. for 24 hours. After the reaction was completed, the resulting mixture was diluted with EA (500 mL) and washed with water (200 mL×5), the organic layer was concentrated under vacuum to afford 2-chloro-4-(2-methyl-4-nitrophenoxy)pyridine (50.0 g) as a yellow solid. LCMS (ESI, m/z): [M+H]⁺=265.0.

### Step 2a. Synthesis of 1-(difluoromethyl)-4-(2-methyl-4-nitrophenoxy)pyridin-2-one

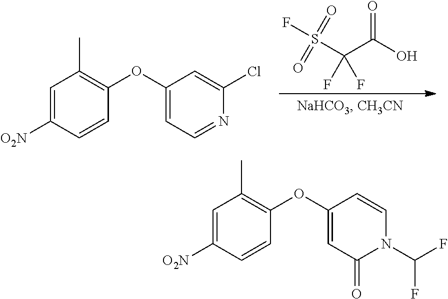

To a solution of 2-chloro-4-(2-methyl-4-nitrophenoxy)pyridine (2.8 g, 10.57 mmol) in CH₃CN (30.0 mL) was added NaHCO₃ (1.7 g, 21.15 mmol) at room temperature. The resulting mixture was stirred at 80° C. for 30 min. Then a solution of difluoro(sulfo)acetic acid (1.8 g, 10.55 mmol) in CH₃CN (10.0 mL) was added to the mixture at room temperature. The mixture was stirred at 80° C. for another 2 h. After the reaction was completed, the resulting mixture was concentrated under reduced pressure. The residue was purified by flash column chromatography with petroleum ether/ethyl acetate (1/1, v/v) to afford 1-(difluoromethyl)-4-(2-methyl-4-nitrophenoxy)pyridin-2-one (1.5 g, 47%) as a light yellow solid. LCMS (ESI, m/z): [M+H]⁺=297.0.

### Step 2b. Alternative Synthesis of 1-(difluoromethyl)-4-(2-methyl-4-nitrophenoxy)pyridin-2-one

In an alternative to Step 2a: To a solution of 2-chloro-4-(2-methyl-4-nitrophenoxy)pyridine (29.0 g) in CH₃CN (300.0 mL) was added NaHCO₃ (18.6 g) at room temperature. The resulting mixture was stirred at 80° C. for 30 min. Then difluoro(sulfo)acetic acid (59.0 g) was added to the mixture at 0° C. The mixture was stirred at 80° C. for another 2 hours. After the reaction was completed, the resulting mixture was filtered. The filtrate was concentrated under reduced pressure. The residue was diluted with EA (500 mL) and washed with water (200 mL×3), the organic layer was concentrated under vacuum to afford 1-(difluoromethyl)-4-(2-methyl-4-nitrophenoxy)pyridin-2-one (30.0 g) as a yellow solid. LCMS (ESI, m/z): [M+H]⁺=297.0.

### Step 3a. Synthesis of 4-(4-amino-2-methylphenoxy)-1-(difluoromethyl)pyridin-2(1H)-one

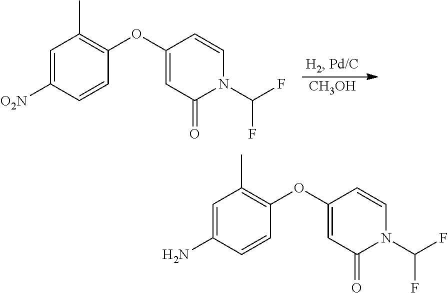

To a solution of 1-(difluoromethyl)-4-(2-methyl-4-nitrophenoxy)pyridin-2-one (1.5 g, 5.06 mmol) in MeOH (20.0 mL) was added Pd/C (500.0 mg, dry). The resulting mixture was stirred at room temperature for 3 h under H₂. After the reaction was completed, the resulting mixture was filtered. The filtrate was concentrated under vacuum to afford 4-(4-amino-2-methylphenoxy)-1-(difluoromethyl)pyridin-2(1H)-one (1.3 g, crude) as a brown solid. LCMS (ESI, m/z): [M+H]⁺=267.0.

### Step 3b. Alternative Synthesis of 4-(4-amino-2-methylphenoxy)-1-(difluoromethyl)pyridin-2(1H)-one

In an alternative to Step 3a: To a solution of 1-(difluoromethyl)-4-(2-methyl-4-nitrophenoxy)pyridin-2-one (6.0 g) in MeOH (80.0 mL) was added Pd/C (2.0 g, dry). The resulting mixture was stirred at room temperature for 3 h under H₂. After the reaction was completed, the resulting mixture was filtered. The filtrate was concentrated under vacuum to afford 4-(4-amino-2-methylphenoxy)-1-(difluoromethyl)pyridin-2(1H)-one (4.4 g, crude) as a brown oil. LCMS (ESI, m/z): [M+H]⁺=267.0.

### Step 4. Synthesis of 4-(4-(6-bromoquinazolin-4-ylamino)-2-methylphenoxy)-1-(difluoromethyl)pyridin-2-one

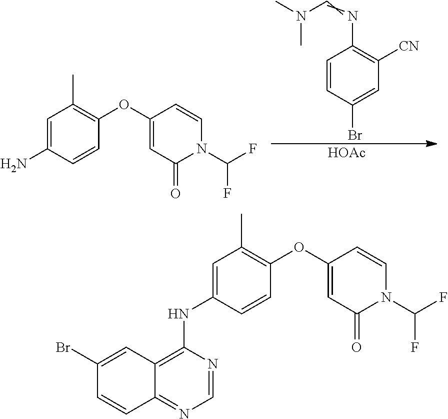

A mixture of 4-(4-amino-2-methylphenoxy)-1-(difluoromethyl)pyridin-2-one (850.0 mg, 3.19 mmol) and N′-(4-bromo-2-cyanophenyl)-N,N-dimethylformimidamide (885.3 mg, 3.51 mmol) in HOAc (10.0 mL) was stirred at 85° C. for 2 h. After the reaction was completed, the resulting mixture was concentrated under reduced pressure. The residue was purified by flash column chromatography with CH₂Cl₂/CH₃OH (10/1, v/v) to afford 4-(4-(6-bromoquinazolin-4-ylamino)-2-methylphenoxy)-1-(difluoromethyl)pyridin-2-one (1.3 g, 86%) as a dark yellow solid. LCMS (ESI, m/z): [M+H]⁺=473.0.

### Step 5. Synthesis of 1-(difluoromethyl)-4-(2-methyl-4-[[6-(3-methylidene-2-oxopyrrolidin-1-yl)quinazolin-4-yl]amino]phenoxy)pyridin-2-one (Compound 12)

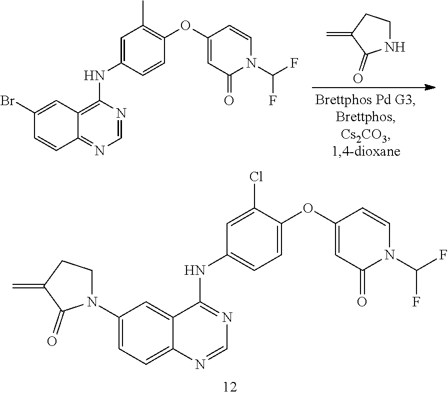

To a mixture of 4-[4-[(6-bromoquinazolin-4-yl)amino]-2-methylphenoxy]-1-(difluoromethyl)pyridin-2-one (300.0 mg, 0.63 mmol) in dioxane (10.0 mL) was added 3-methylidenepyrrolidin-2-one (184.6 mg, 1.90 mmol), Cs₂CO₃ (1239.1 mg, 3.80 mmol), Brettphos Pd G3 (114.9 mg, 0.12 mmol) and BrettPhos (136.1 mg, 0.25 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (9/1, v/v) and then purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 25% B to 35% B in 8 min; 254/220 nm) to afford 1-(difluoromethyl)-4-(2-methyl-4-[[6-(3-methylidene-2-oxopyrrolidin-1-yl)quinazolin-4-yl]amino]phenoxy)pyridin-2-one (Compound 12) (23.6 mg, 7%) as a white solid. LCMS (ESI, m/z): [M+H]⁺=490.2. ¹H NMR (300 MHz, DMSO-d₆): δ 9.86 (s, 1H), 8.70-8.66 (m, 1H), 8.57 (s, 1H), 8.34 (d, J=2.4 Hz, 1H), 7.98-7.58 (m, 5H), 7.22-7.19 (m, 1H), 6.38-6.34 (m, 1H), 5.98-5.96 (m, 1H), 5.54 (d, J=0.9 Hz, 1H), 5.34 (d, J=2.7 Hz, 1H), 4.06-4.02 (m, 2H), 2.99-2.94 (m, 2H), 2.16 (s, 3H).

### Example S13: Synthesis of 1-[4 [(3-methyl-4-[[1,2,4]triazolo[1,5-a]pyridin-7-ylsulfanyl]phenyl)amino]quinazolin-6-yl]-3-methylidenepyrrolidin-2-one (Compound 13)

### Step 1. Synthesis of 7-[[(4-methoxyphenyl)methyl]sulfanyl]-[1,2,4]triazolo[1,5-a]pyridine

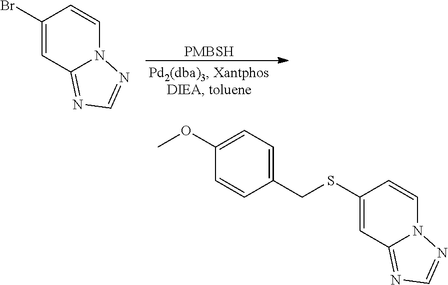

To a mixture of 7-bromo-[1,2,4]triazolo[1,5-a]pyridine (5.0 g, 25.25 mmol) in toluene (25.0 mL) was added (4-methoxyphenyl)methanethiol (PMBSH, 3.9 g, 25.25 mmol), DIEA (6.5 g, 50.50 mmol), XantPhos (1.5 g, 2.53 mmol) and Pd₂(dba)₃ (1.1 g, 1.26 mmol) at room temperature. The resulting mixture was stirred at 100° C. for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (1/10, v/v) to afford 7-[[(4-methoxyphenyl)methyl]sulfanyl]-[1,2,4]triazolo[1,5-a]pyridine (6.0 g, 87%) as an orange solid. LCMS (ESI, m/z): [M+H]⁺=272.1.

### Step 2. Synthesis of [1,2,4]triazolo[1,5-a]pyridine-7-thiol

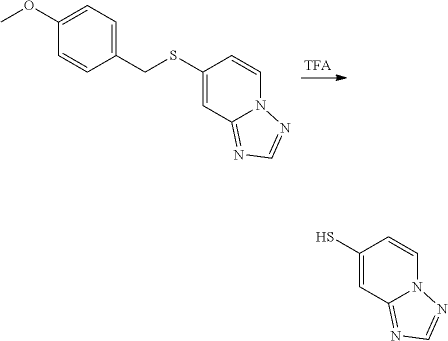 [SMILES: COc1ccc(CSc2ccn3ncnc3c2)cc1 ; Sc1ccn2ncnc2c1]

A solution of 7-[[(4-methoxyphenyl)methyl]sulfanyl]-[1,2,4]triazolo[1,5-a]pyridine (1.5 g, 5.53 mmol) in TFA (60.0 mL) was stirred at 120° C. for 16 h. After the reaction was completed, the resulting mixture was concentrated under reduced pressure to afford [1,2,4]triazolo[1,5-a]pyridine-7-thiol (2.6 g, crude) as a tan oil. LCMS (ESI, m/z): [M+H]⁺=152.2.

### Step 3. Synthesis of 7-[(2-methyl-4-nitrophenyl)sulfanyl]-[1,2,4]triazolo[1,5-a]pyridine

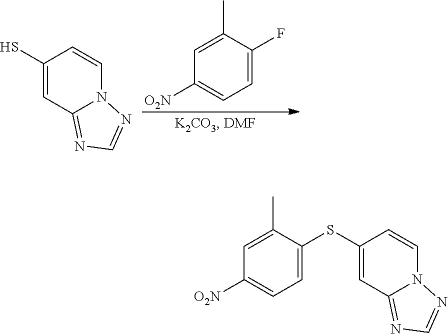

To a mixture of [1,2,4]triazolo[1,5-a]pyridine-7-thiol (1.8 g, crude) in DMF (20.0 mL) was added 1-fluoro-2-methyl-4-nitrobenzene (1.8 g, 11.91 mmol) and K₂CO₃ (3.3 g, 23.81 mmol) at room temperature. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 7-[(2-methyl-4-nitrophenyl)sulfanyl]-[1,2,4]triazolo[1,5-a]pyridine (800.0 mg, 23%) as a white solid. LCMS (ESI, m/z): [M+H]⁺=287.1.

### Step 4. Synthesis of 3-methyl-4-[[1,2,4]triazolo[1,5-a]pyridin-7-ylsulfanyl]aniline

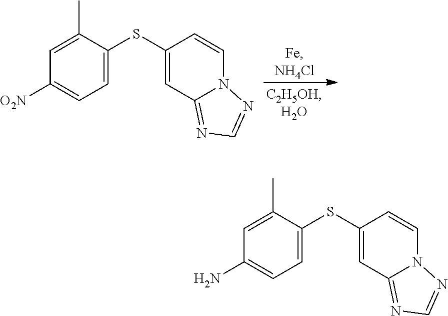 [SMILES: Cc1cc([N+](=O)[O-])ccc1Sc1ccn2ncnc2c1 ; Cc1cc(N)ccc1Sc1ccn2ncnc2c1]

To a solution of 7-[(2-methyl-4-nitrophenyl)sulfanyl]-[1,2,4]triazolo[1,5-a]pyridine (800.0 mg, 2.79 mmol) in EtOH (16.0 mL)/H₂O (3.2 mL) was added NH₄Cl (747.3 mg, 13.97 mmol). Then Fe (780.2 mg, 13.97 mmol) was added to the mixture at 80° C. The resulting mixture was stirred at 80° C. for 3 h. After the reaction was completed, the resulting mixture was filtered. The filtrate was concentrated under reduced pressure. The residue was purified by flash column chromatography with CH₂Cl₂/MeOH (10/1, v/v) to afford 3-methyl-4-[[1,2,4]triazolo[1,5-a]pyridin-7-ylsulfanyl]aniline (500.0 mg, 69%) as a brown oil. LCMS (ESI, m/z): [M+H]⁺=257.1.

### Step 5. Synthesis of 6-bromo-N-(3-methyl-4-[[1,2,4]triazolo[1,5-a]pyridin-7-ylsulfanyl]phenyl)quinazolin-4-amine

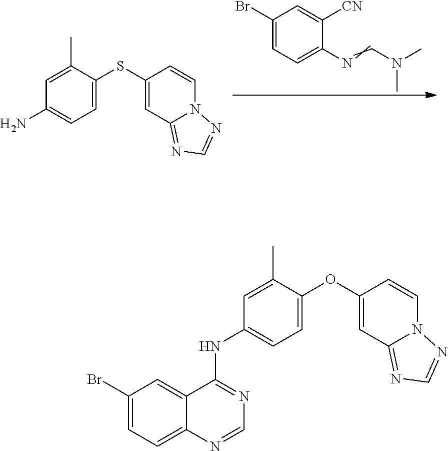 [SMILES: Cc1cc(N)ccc1Sc1ccn2ncnc2c1 ; CN(C)C=Nc1ccc(Br)cc1C#N ; Cc1cc(Nc2ncnc3ccc(Br)cc23)ccc1Oc1ccn2ncnc2c1]

To a solution of 3-methyl-4-[[1,2,4]triazolo[1,5-a]pyridin-7-ylsulfanyl]aniline (255.0 mg, 0.10 mmol) in HOAc (10.0 mL) was added N′-(4-bromo-2-cyanophenyl)-N,N-dimethylformimidamide (250.8 mg, 0.10 mmol) at room temperature. The resulting solution was stirred at 85° C. for 2 h. After the reaction was completed, the resulting mixture was concentrated under vacuo. The residue was purified by flash column chromatography with CH₂Cl₂/MeOH (10/1, v/v) to afford 6-bromo-N-(3-methyl-4-[[1,2,4]triazolo[1,5-a]pyridin-7-ylsulfanyl]phenyl)quinazolin-4-amine (100.0 mg, 21%) as a light yellow solid. LCMS (ESI, m/z): [M+H]⁺=463.3.

### Step 6. Synthesis of 1-[4-[(3-methyl-4-[[1,2,4]triazolo[1,5-a]pyridin-7-ylsulfanyl]phenyl)amino]quinazolin-6-yl]-3-methylidenepyrrolidin-2-one (Compound 13)

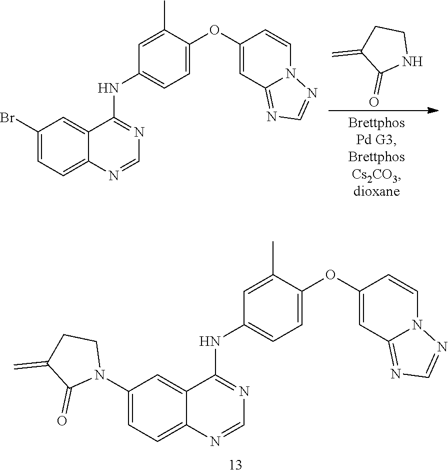

To a solution of 6-bromo-N-(3-methyl-4-[[1,2,4]triazolo[1,5-a]pyridin-7-ylsulfanyl]phenyl)quinazolin-4-amine (78.0 mg, 0.17 mmol) in dioxane (4.0 mL) was added 3-methylidenepyrrolidin-2-one (49.0 mg, 0.51 mmol), BrettPhos Pd G3 (15.3 mg, 0.02 mmol), BrettPhos (18.1 mg, 0.03 mmol) and Cs₂CO₃ (329.1 mg, 1.01 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 37% B to 47% B in 8 min 254/220 nm; to afford 1-[4-[(3-methyl-4-[[1,2,4]triazolo[1,5-a]pyridin-7-ylsulfanyl]phenyl)amino]quinazolin-6-yl]-3-methylidenepyrrolidin-2-one (Compound 13) (21.9 mg, 26%) as a light yellow solid. LCMS (ESI, m/z): [M+H]⁺=480.2. ¹H NMR (300 MHz, DMSO-d₆): δ 9.97 (s, 1H), 8.87-8.84 (m, 1H), 8.74-8.66 (m, 2H), 8.41-8.38 (m, 2H), 7.99-7.96 (m, 2H), 7.88 (d, J=9.3 Hz, 1H), 7.69-7.66 (m, 1H), 7.07 (d, J=1.2 Hz, 1H), 6.96-6.93 (m, 1H), 6.00-5.99 (m, 1H), 5.56 (d, J=0.9 Hz, 1H), 4.10-4.05 (m, 2H), 3.01-2.96 (m, 2H), 2.41 (s, 3H).

### Example S14: Synthesis of 1-[4 [(2 fluoro-3-methyl-4-[[1,2,4]triazolo[1,5-a]pyridin-7-yloxy]phenyl)amino]quinazolin-6-yl]-3-methylidenepyrrolidin-2-one (Compound 14)

### Step 1. Synthesis of 7-(3-fluoro-2-methyl-4-nitrophenoxy)-[1,2,4]triazolo[1,5-a]pyridine

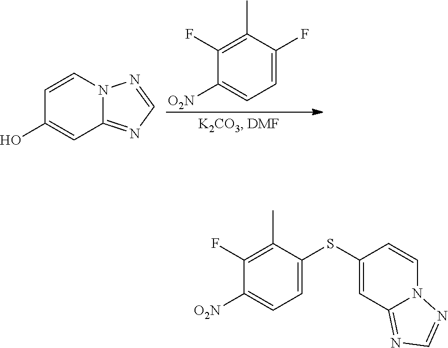

To a solution of [1,2,4]triazolo[1,5-a]pyridin-7-ol (2.0 g, 14.80 mmol) in DMF (20.0 mL) was added 1,3-difluoro-2-methyl-4-nitrobenzene (5.1 g, 29.60 mmol) and K₂CO₃ (12.8.0 g, 88.78 mmol) at room temperature. The resulting mixture was stirred at room temperature for 4 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (2/1, v/v) to afford 7-(3-fluoro-2-methyl-4-nitrophenoxy)-[1,2,4]triazolo[1,5-a]pyridine (700.0 mg, 16%) as a white solid. LCMS (ESI, m/z): [M+H]+=289.1.

### Step 2. Synthesis of 4-([1,2,4]triazolo[1,5-a]pyridin-7-yloxy)-2-fluoro-3-methylaniline

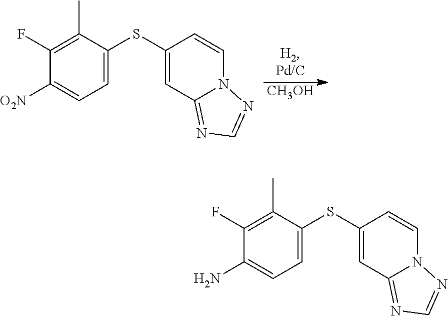

To a solution of 7-(3-fluoro-2-methyl-4-nitrophenoxy)-[1,2,4]triazolo[1,5-a]pyridine (700.0 mg, 2.42 mmol) in MeOH (10.0 mL) was added Pd/C (129.2 mg, dry) at room temperature under N₂. The resulting mixture was stirred at room temperature for 2 h under H2. After the reaction was completed, the resulting mixture was filtered. The filtrate was concentrated under reduced pressure to afford 4-([1,2,4]triazolo[1,5-a]pyridin-7-yloxy)-2-fluoro-3-methylaniline (380.0 mg, crude) as a white solid. LCMS (ESI, m/z): [M+H]⁺=259.1.

### Step 3. Synthesis of 6-bromo-N-(2-fluoro-3-methyl-4-[[1,2,4]triazolo[1,5-a]pyridin-7-yloxy]phenyl)quinazolin-4-amine

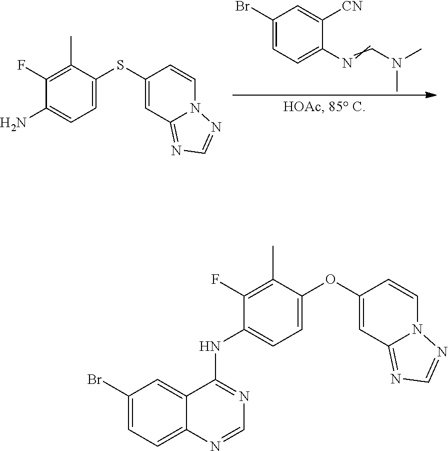 [SMILES: Cc1c(Sc2ccn3ncnc3c2)ccc(N)c1F ; CN(C)C=Nc1ccc(Br)cc1C#N ; Cc1c(Oc2ccn3ncnc3c2)ccc(Nc2ncnc3ccc(Br)cc23)c1F]

To a solution of 4-([1,2,4]triazolo[1,5-a]pyridin-7-yloxy)-2-fluoro-3-methylaniline (220.0 mg, 0.85 mmol) in HOAc (25.0 mL) was added N′-(4-bromo-2-cyanophenyl)-N,N-dimethylformimidamide (214.8 mg, 0.85 mmol) at room temperature. The mixture was stirred at 85° C. for 2 h. After the reaction was completed, the mixture was concentrated under vacuum. The residue was purified by flash column chromatography with CH₂Cl₂/MeOH (10/1, v/v) to afford 6-bromo-N-(2-fluoro-3-methyl-4-[[1,2,4]triazolo[1,5-a]pyridin-7-yloxy]phenyl)quinazolin-4-amine (390.0 mg, 98%) as a grey solid. LCMS (ESI, m/z): [M+H]+=465.0.

### Step 4. Synthesis of 1-[4-[(2-fluoro-3-methyl-4-[[1,2,4]triazolo[1,5-a]pyridin-7-yloxy]phenyl)amino]quinazolin-6-yl]-3-methylidenepyrrolidin-2-one (Compound 14)

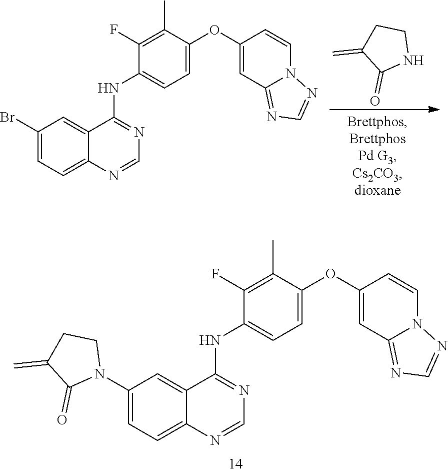

To a solution of 6-bromo-N-(2-fluoro-3-methyl-4-[[1,2,4]triazolo[1,5-a]pyridin-7-yloxy]phenyl)quinazolin-4-amine (350.0 mg, 0.75 mmol) in dioxane (10.0 mL) was added 3-methylidenepyrrolidin-2-one (219.2 mg, 2.26 mmol), Cs₂CO₃ (1470.5 mg, 4.51 mmol), BrettPhos Pd G3 (68.2 mg, 0.08 mmol) and BrettPhos (80.8 mg, 0.15 mmol) at room temperature. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with the following conditions: Column: Xselect CSH OBD Column 30×150 mm, 5 um; Mobile Phase A: Water (0.1% FA), Mobile Phase B: ACN; Flow rate: 60 mL/min; Gradient: 19% B to 29% B in 9 min, 254/220 nm to afford 1-[4-[(2-fluoro-3-methyl-4-[[1,2,4]triazolo[1,5-a]pyridin-7-yloxy]phenyl)amino]quinazolin-6-yl]-3-methylidenepyrrolidin-2-one (Compound 14) (9.5 mg, 2%) as a white solid. LCMS (ESI, m/z): [M+H]⁺=482.2. ¹H NMR (300 MHz, DMSO-d₆): δ 9.93 (s, 1H), 8.99 (d, J=7.5 Hz, 1H), 8.73-8.70 (m, 1H), 8.51 (s, 1H), 8.43 (s, 1H), 8.36 (s, 1H), 7.87-7.84 (m, 1H), 7.54-7.48 (m, 1H), 7.14-7.08 (m, 2H), 6.97 (d, J=2.4 Hz, 1H), 5.99 (s, 1H), 5.55 (s, 1H), 4.07-4.03 (m, 2H), 3.03-2.93 (m, 2H), 2.18 (s, 3H).

### Example S15: Synthesis of 1-[4 [(2-fluoro-5-methyl-4-[[1,2,4]triazolo[1,5-a]pyridin-7-yloxy]phenyl)amino]quinazolin-6-yl]-3-methylidenepyrrolidin-2-one (Compound 15)

### Step 1. Synthesis of [1,2,4]triazolo[1,5-a]pyridin-7-ol

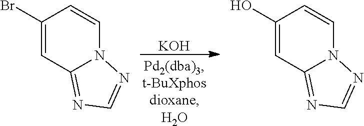

To a solution of 7-bromo-[1,2,4]triazolo[1,5-a]pyridine (501.0 mg, 2.53 mmol) in 1,4-dioxane (8.0 mL)/H₂O (2.0 mL) was added KOH (625.7 mg, 11.15 mmol), Pd₂(dba)₃ (263.1 mg, 0.28 mmol) and t-BuXPhos (231.1 mg, 0.54 mmol) at room temperature. The resulting mixture was stirred at 100° C. for 4 h. After the reaction was completed, the mixture was cooled to room temperature and then extracted with EtOAc. The pH value of the aqueous layer was adjusted to pH 5 with 1N HCl. The mixture was evaporated in vacuo. The residue was purified by flash column chromatography with CH₂Cl₂/MeOH (5/1, v/v) to afford [1,2,4]triazolo[1,5-a]pyridin-7-ol (140.0 mg, 40%) as a white solid. LCMS (ESI, m/z): [M+H]⁺=136.0.

### Step 2. Synthesis of 7-(5-fluoro-2-methyl-4-nitrophenoxy)-[1,2,4]triazolo[1,5-a]pyridine

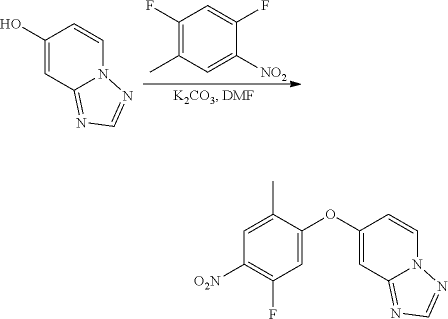

To a solution of [1,2,4]triazolo[1,5-a]pyridin-7-ol (1.3 g, 9.62 mmol) in DMF (30.0 mL) was added 1,5-difluoro-2-methyl-4-nitrobenzene (5.0 g, 28.86 mmol) and K₂CO₃ (8.0 g, 57.72 mmol) at room temperature. The resulting mixture was stirred at room temperature for 4 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (2/1, v/v) to afford 7-(5-fluoro-2-methyl-4-nitrophenoxy)-[1,2,4]triazolo[1,5-a]pyridine (1.1 g, 39%) as a white solid. LCMS (ESI, m/z): [M+H]⁺=289.1.

### Step 3. Synthesis of 2-fluoro-5-methyl-4-[[1,2,4]triazolo[1,5-a]pyridin-7-yloxy]aniline

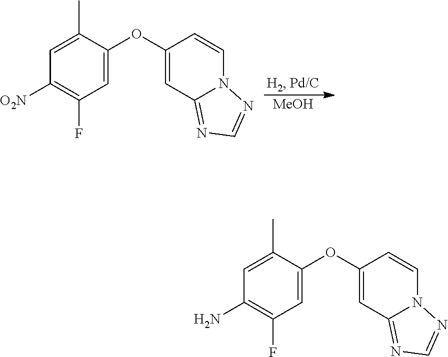 [SMILES: "MeOH" ; Cc1cc([N+](=O)[O-])c(F)cc1Oc1ccn2ncnc2c1 ; Cc1cc(N)c(F)cc1Oc1ccn2ncnc2c1]

To a solution of 7-(5-fluoro-2-methyl-4-nitrophenoxy)-[1,2,4]triazolo[1,5-a]pyridine (500.0 mg, 1.73 mmol) in MeOH (10.0 mL) was added Pd/C (150.0 mg, dry) at room temperature. The resulting mixture was stirred at room temperature for 2 h under H2. After the reaction was completed, the resulting mixture was filtered. The filtrate was concentrated under reduced pressure to afford 2-fluoro-5-methyl-4-[[1,2,4]triazolo[1,5-a]pyridin-7-yloxy]aniline (380.0 mg, crude) as a white solid. LCMS (ESI, m/z): [M+H]⁺=259.1.

### Step 4. Synthesis of 6-bromo-N-(2-fluoro-5-methyl-4-[[1,2,4]triazolo[1,5-a]pyridin-7-yloxy]phenyl)quinazolin-4-amine

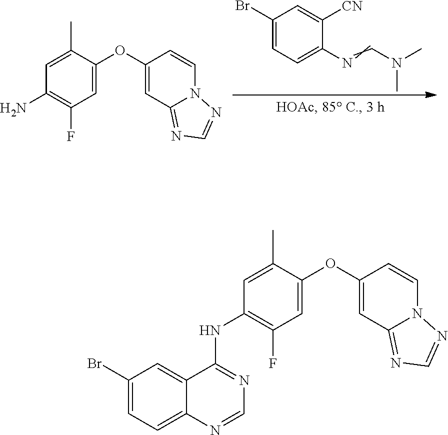 [SMILES: Cc1cc(N)c(F)cc1Oc1ccn2ncnc2c1 ; CN(C)C=Nc1ccc(Br)cc1C#N ; Cc1cc(Nc2ncnc3ccc(Br)cc23)c(F)cc1Oc1ccn2ncnc2c1]

To a solution of 2-fluoro-5-methyl-4-[[1,2,4]triazolo[1,5-a]pyridin-7-yloxy]aniline (170.0 mg, 0.66 mmol) in HOAc (10.0 mL) was added N′-(4-bromo-2-cyanophenyl)-N,N-dimethylformimidamide (166.0 mg, 0.66 mmol) at room temperature. The resulting mixture was stirred at 85° C. for 2 h. After the reaction was completed, the resulting mixture was concentrated under vacuo. The residue was purified by flash column chromatography with CH₂Cl₂/MeOH (10/1, v/v) to afford 6-bromo-N-(2-fluoro-5-methyl-4-[[1,2,4]triazolo[1,5-a]pyridin-7-yloxy]phenyl)quinazolin-4-amine (260.0 mg, 84%) as a light yellow solid. LCMS (ESI, m/z): [M+H]⁺=465.2.

### Step 5. Synthesis of 1-[4-[(2-fluoro-5-methyl-4-[[1,2,4]triazolo[1,5-a]pyridin-7-yloxy]phenyl)amino]quinazolin-6-yl]-3-methylidenepyrrolidin-2-one (Compound 15)

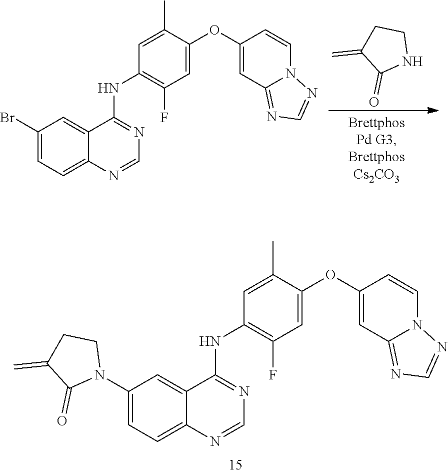

To a solution of 6-bromo-N-(2-fluoro-5-methyl-4-[[1,2,4]triazolo[1,5-a]pyridin-7-yloxy]phenyl)quinazolin-4-amine (230.0 mg, 0.49 mmol) in dioxane (9.0 mL) was added 3-methylidenepyrrolidin-2-one (288.0 mg, 2.96 mmol), Cs₂CO₃ (966.4 mg, 2.97 mmol), BrettPhos (53.1 mg, 0.10 mmol) and Brettphos Pd G3 (44.8 mg, 0.05 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 32% B to 42% B in 8 min, 254/220 nm to afford 1-[4-[(2-fluoro-5-methyl-4-[[1,2,4]triazolo[1,5-a]pyridin-7-yloxy]phenyl)amino]quinazolin-6-yl]-3-methylidenepyrrolidin-2-one (Compound 15) (15.8 mg, 6%) as a white solid. LCMS (ESI, m/z): [M+H]⁺=482.0. ¹H NMR (300 MHz, CD₃OD): δ 8.80 (d, J=7.5 Hz, 1H), 8.61-8.57 (m, 1H), 8.50-8.46 (m, 2H), 8.34 (s, 1H), 7.90 (d, J=9.0 Hz, 1H), 7.65 (d, J=8.4 Hz, 1H), 7.20-7.12 (m, 2H), 7.02 (d, J=2.4 Hz, 1H), 6.15-6.13 (m, 1H), 5.62-5.60 (m, 1H), 4.16-4.12 (m, 2H), 3.09-3.04 (m, 2H), 2.26 (s, 3H).

### Example S16: Synthesis of 1-[4 [(3-methyl-4-[[1,2,4]triazolo[1,5-a]pyridin-7-ylmethyl]phenyl)amino]quinazolin-6-yl]-3-methylidenepyrrolidin-2-one (Compound 16)

### Step 1. Synthesis of tert-butyl N-[3-methyl-4-(4,4,5,5-tetramethyl-1,3,2-dioxaborolan-2-yl)phenyl]carbamate

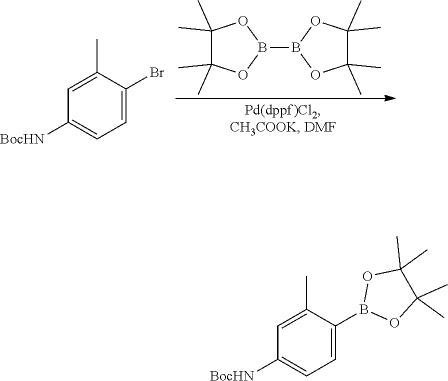

To a solution of tert-butyl N-(4-bromo-3-methylphenyl)carbamate (3.0 g, 10.48 mmol) and bis(pinacolato)diboron (3.99 g, 15.71 mmol) in DMF (60.0 mL) was added KOAc (3.0 g, 31.45 mmol) and Pd(dppf)Cl₂ (770.0 mg, 1.05 mmol) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (7/3, v/v) to afford tert-butyl N-[3-methyl-4-(4,4,5,5-tetramethyl-1,3,2-dioxaborolan-2-yl)phenyl]carbamate (3.5 g, 90%) as an off-white solid. LCMS (ESI, m/z): [M+H]⁺=334.2.

### Step 2. Synthesis of methyl 2-(((hydroxyamino)methylene)amino)isonicotinate

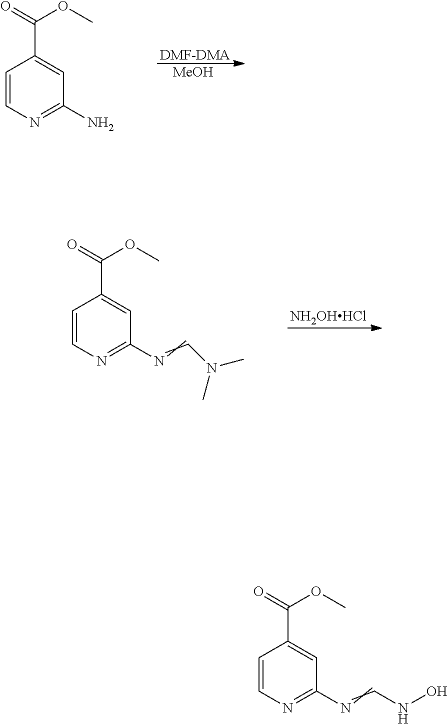

To a solution of methyl 2-aminoisonicotinate (20.0 g, 131.44 mmol) in CH₃OH (200.0 mL) was added DMF-DMA (23.5 g, 197.21 mmol) at room temperature. The resulting mixture was stirred at 75° C. for 3 h. Then hydroxylamine hydrochloride (25.8 g, 371.55 mmol) was added to the mixture at room temperature. The resulting mixture was stirred at 50° C. for another 1 h. After the reaction was completed, the reaction mixture was cooled to room temperature and filtered. The solid was washed with CH₃OH and dried to afford methyl 2-(((hydroxyamino)methylene)amino)isonicotinate (18.9 g, crude) as a white solid. LCMS (ESI, m/z): [M+H]⁺=196.0.

### Step 3. Synthesis of methyl [1,2,4]triazolo[1,5-a]pyridine-7-carboxylate

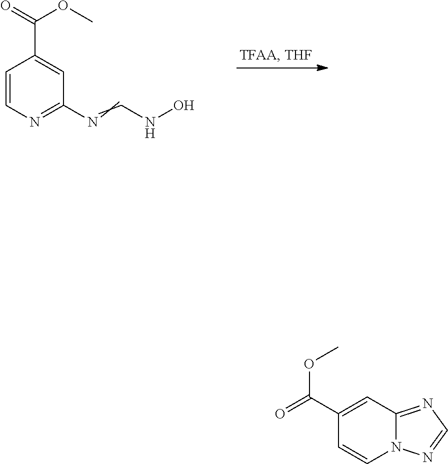

To a solution of methyl 2-(((hydroxyamino)methylene)amino)isonicotinate (18.9 g, crude) in THE (200.0 mL) was added TFAA (22.3 g, 106.50 mmol) at room temperature. The resulting mixture was stirred at room temperature for 5 h. After the reaction was completed, the pH value of the mixture was adjusted to 7 with saturated NaHCO₃(aq.). The resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford methyl [1,2,4]triazolo[1,5-a]pyridine-7-carboxylate (13.7 g, 79%) as a white solid. LCMS (ESI, m/z): [M+H]⁺=178.1.

### Step 4. Synthesis of [1,2,4]triazolo[1,5-a]pyridin-7-ylmethanol

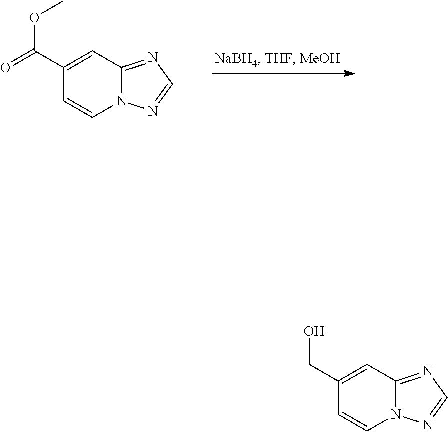

To a solution of methyl [1,2,4]triazolo[1,5-a]pyridine-7-carboxylate (13.7 g, 77.33 mmol) in THF (150.0 mL)/CH₃OH (15 mL) was added NaBH₄ (14.6 g, 386.65 mmol) at 0° C. under N₂. The resulting mixture was stirred at room temperature for 4 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (9/1, v/v) to afford [1,2,4]triazolo[1,5-a]pyridin-7-ylmethanol (4.8 g, 41%) as a white solid. LCMS (ESI, m/z): [M+H]⁺=150.1.

### Step 5. Synthesis of 7-(bromomethyl)-[1,2,4]triazolo[1,5-a]pyridine

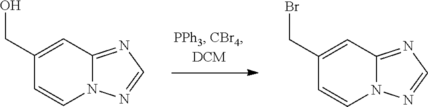 [SMILES: OCc1ccn2ncnc2c1 ; BrCc1ccn2ncnc2c1]

To a mixture of [1,2,4]triazolo[1,5-a]pyridin-7-ylmethanol (2.5 g, 16.76 mmol) and PPh₃ (6.5 g, 25.14 mmol) in CH₂Cl₂ (30.0 mL) was added CBr₄ (8.3 g, 25.14 mmol) at 0° C. under N₂. The resulting mixture was stirred at room temperature for 3 h. After the reaction was completed, the mixture was concentrated under reduced pressure. The residue was purified by flash column chromatography with CH₂Cl₂/CH₃OH (10/1, v/v) to afford 7-(bromomethyl)-[1,2,4]triazolo[1,5-a]pyridine (1.5 g, 42%) as a white solid. LCMS (ESI, m/z): [M+H]⁺=212.0.

### Step 6. Synthesis of tert-butyl N-(3-methyl-4-[[1,2,4]triazolo[1,5-a]pyridin-7-ylmethyl]phenyl)carbamate

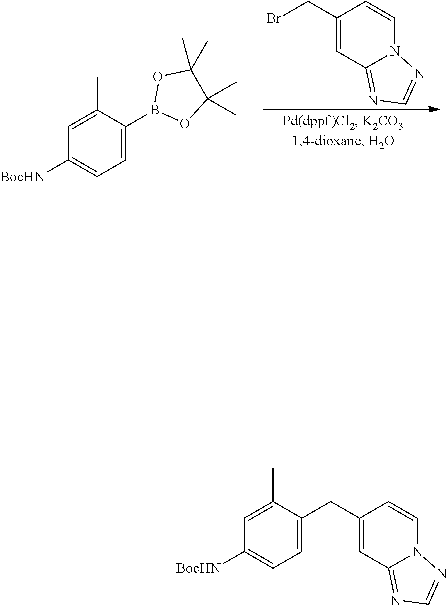 [SMILES: Cc1cc(NC(=O)OC(C)(C)C)ccc1B1OC(C)(C)C(C)(C)O1 ; BrCc1ccn2ncnc2c1 ; Cc1cc(NC(=O)OC(C)(C)C)ccc1Cc1ccn2ncnc2c1]

To a solution of tert-butyl N-[3-methyl-4-(4,4,5,5-tetramethyl-1,3,2-dioxaborolan-2-yl)phenyl]carbamate (1.6 g, 4.71 mmol) in dioxane (15.0 mL)/H₂O (1.5 mL) was added K₂CO₃ (2.0 g, 14.10 mmol), 7-(bromomethyl)-[1,2,4]triazolo[1,5-a]pyridine (1.0 g, 4.72 mmol) and Pd(dppf)Cl₂ (690.0 mg, 0.47 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford tert-butyl N-(3-methyl-4-[[1,2,4]triazolo[1,5-a]pyridin-7-ylmethyl]phenyl)carbamate (900.0 mg, 56%) as a white solid. LCMS (ESI, m/z): [M+H]⁺=339.0.

### Step 7. Synthesis of 3-methyl-4-[[1,2,4]triazolo[1,5-a]pyridin-7-ylmethyl]aniline

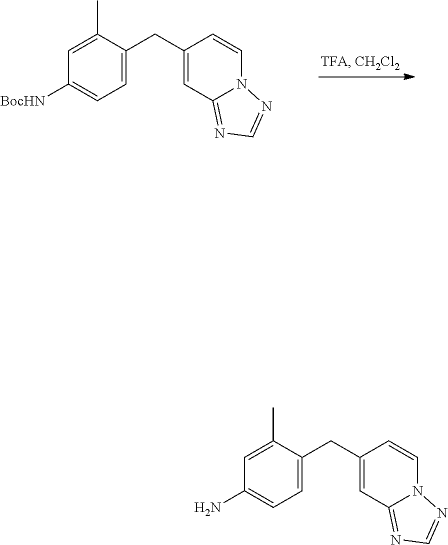 [SMILES: Cc1cc(NC(=O)OC(C)(C)C)ccc1Cc1ccn2ncnc2c1 ; Cc1cc(N)ccc1Cc1ccn2ncnc2c1]

To a solution of tert-butyl N-(3-methyl-4-[[1,2,4]triazolo[1,5-a]pyridin-7-ylmethyl]phenyl)carbamate (900.0 mg, 2.66 mmol) in CH₂Cl₂ (5.0 mL) was added TFA (5.0 mL) at room temperature. The resulting mixture was stirred at room temperature for 2 h. The pH value of the resulting mixture was adjusted to 8 with saturated NaHCO₃ solution and then concentrated under vacuum. The residue was purified by reverse phase flash column chromatography with acetonitrile/water (4/6, v/v) to afford 3-methyl-4-[[1,2,4]triazolo[1,5-a]pyridin-7-ylmethyl]aniline (600.0 mg, 94%) as an off-white solid. LCMS (ESI, m/z): [M+H]⁺=239.0.

### Step 8. Synthesis of 6-bromo-N-(3-methyl-4-[[1,2,4]triazolo[1,5-a]pyridin-7-ylmethyl]phenyl)quinazolin-4-amine

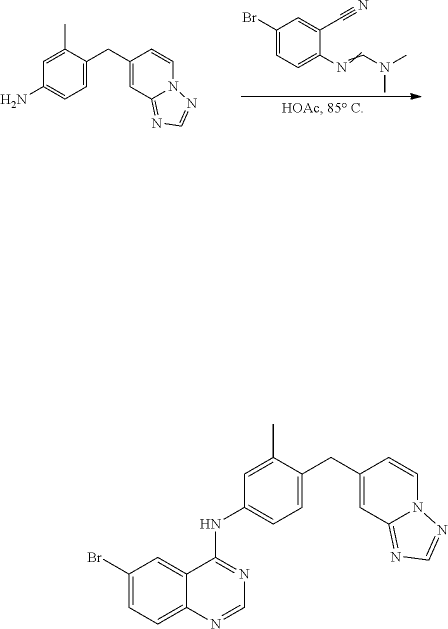 [SMILES: Cc1cc(N)ccc1Cc1ccn2ncnc2c1 ; CN(C)C=Nc1ccc(Br)cc1C#N ; Cc1cc(Nc2ncnc3ccc(Br)cc23)ccc1Cc1ccn2ncnc2c1]

To a solution of 3-methyl-4-[[1,2,4]triazolo[1,5-a]pyridin-7-ylmethyl]aniline (200.0 mg, 0.83 mmol) in HOAc (5.0 mL) was added N′-(4-bromo-2-cyanophenyl)-N,N-dimethylformimidamide (211.6 mg, 0.83 mmol) at room temperature. The resulting mixture was stirred at 85° C. for 2 h. After the reaction was completed, the mixture was concentrated under reduced pressure. The residue was purified by flash column chromatography with CH₂Cl₂/CH₃OH (9/1, v/v) to afford 6-bromo-N-(3-methyl-4-[[1,2,4]triazolo[1,5-a]pyridin-7-ylmethyl]phenyl)quinazolin-4-amine (200.0 mg, 53%) as an off-white solid. LCMS (ESI, m/z): [M+H]⁺=445.1

### Step 9. Synthesis of 1-[4-[(3-methyl-4-[[1,2,4]triazolo[1,5-a]pyridin-7-ylmethyl]phenyl)amino]quinazolin-6-yl]-3-methylidenepyrrolidin-2-one (Compound 16)

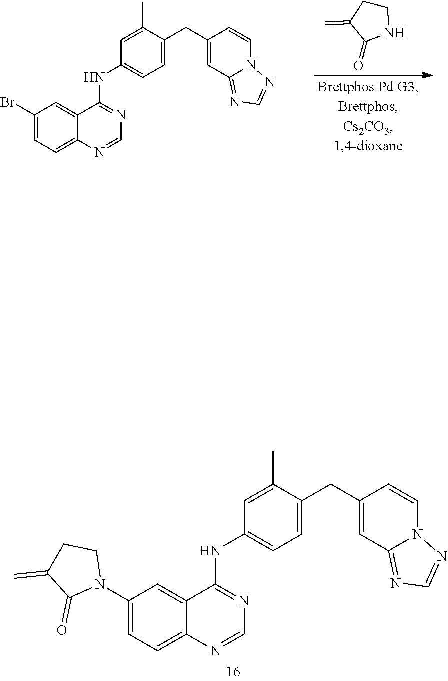

To a solution of 6-bromo-N-(3-methyl-4-[[1,2,4]triazolo[1,5-a]pyridin-7-ylmethyl]phenyl)quinazolin-4-amine (200.0 mg, 0.44 mmol) in dioxane (5.0 mL) was added 3-methylidenepyrrolidin-2-one (130.8 mg, 1.35 mmol), Cs₂CO₃ (877.96 mg, 2.70 mmol), BrettPhos (96.42 mg, 0.18 mmol) and Brettphos Pd G3 (81.42 mg, 0.09 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (9/1, v/v) and then purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 17% B to 28% B in 8 min; 254/220 nm) to afford 1-[4-[(3-methyl-4-[[1,2,4]triazolo[1,5-a]pyridin-7-ylmethyl]phenyl)amino]quinazolin-6-yl]-3-methylidenepyrrolidin-2-one (Compound 16) (6.3 mg, 3%) as a light-yellow solid. LCMS (ESI, m/z): [M+H]⁺=462.2. ¹H NMR (400 MHz, DMSO-d₆): δ 9.78 (s, 1H), 8.86 (d, J=6.8 Hz, 1H), 8.70-8.65 (m, 1H), 8.53 (s, 1H), 8.43 (s, 1H), 8.33 (d, J=2.0 Hz, 1H), 7.82 (d, J=9.2 Hz, 1H), 7.66-7.63 (m, 1H), 7.61 (s, 1H), 7.52 (s, 1H), 7.29-7.24 (m, 1H), 7.05-7.00 (m, 1H), 6.00 (s, 1H), 5.46 (s, 1H), 4.14 (s, 2H), 4.08-4.02 (m, 2H), 3.05-2.95 (m, 2H), 2.27 (s, 3H).

### Example S17: Synthesis of (Z)-1-(4-(4-([1,2,4]triazolo[1,5-a]pyridin-7 yloxy)-3-methylphenylamino)quinazolin-6-yl)-3-(2-(dimethylamino)ethylidene)pyrrolidin-2-one and (E)-1-(4-(4-([1,2,4]triazolo[1,5-a]pyridin-7-yloxy)-3-methylphenylamino) quinazolin-6-yl)-3-(2-(dimethylamino)ethylidene)pyrrolidin-2-one (Compounds 17 and 18)

### Step 1. Synthesis of 2,4-dibromobutanamide

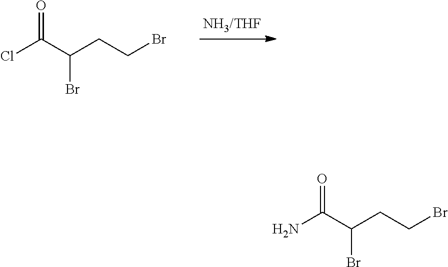 [SMILES: O=C(Cl)C(Br)CCBr ; NC(=O)C(Br)CCBr]

To a solution of NH₃(g) in THF (150.0 mL, 0.5 mol/L) was added dropwise 2,4-dibromobutanoyl chloride (8.6 g, 32.53 mmol) at room temperature. The mixture was stirred at room temperature for 1 h. After the reaction was completed, the mixture was evaporated in vacuo to afford 2,4-dibromobutanamide (9.7 g, crude) as a light yellow oil. LCMS (ESI, m/z): [M+H]⁺=243.9.

### Step 2. Synthesis of 3-bromopyrrolidin-2-one

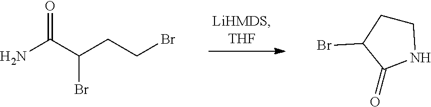 [SMILES: NC(=O)C(Br)CCBr ; O=C1NCCC1Br]

To a solution of 2,4-dibromobutanamide (9.7 g, crude) in THF (250.0 mL) was added LiHMDS (35.0 mL, 1.3 mol/L) at 0° C. under N₂. The mixture was stirred at 0° C. for 2.5 h. After the reaction was completed, the mixture was diluted with H₂O and extracted with EtOAc. The combined organic layer was washed with brine, dried over anhydrous sodium sulfate and filtered. The filtrate was concentrated under vacuum to afford 3-bromopyrrolidin-2-one (2.4 g, crude) as a light yellow oil. LCMS (ESI, m/z): [M+H]⁺=164.0.

### Step 3. Synthesis of 1-acetyl-3-bromopyrrolidin-2-one

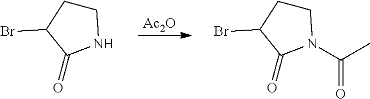

A mixture of 3-bromopyrrolidin-2-one (2.4 g, 14.63 mmol) in Ac₂O (50.0 mL) was heated at 110° C. for 5 h. After the reaction was completed, the mixture was evaporated in vacuo. The residue was purified by flash column chromatography with petroleum ether/EtOAc (21/79, v/v) to afford 1-acetyl-3-bromopyrrolidin-2-one (1.5 g, 49%) as a colorless oil. LCMS (ESI, m/z): [M+H]⁺=206.0.

### Step 4. Synthesis of 1-acetyl-3-(triphenyl-lambda5-phosphanylidene)pyrrolidin-2-one

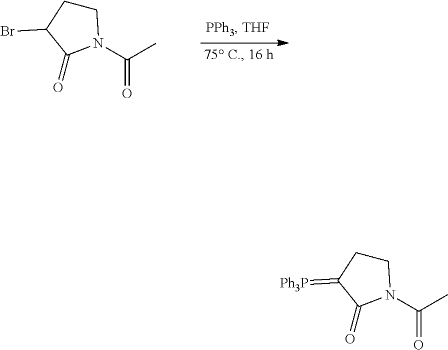

A mixture of 1-acetyl-3-bromopyrrolidin-2-one (1.5 g, 7.28 mmol) and PPh₃ (2.3 g, 8.76 mmol) in THF (30.0 mL) was heated at 80° C. for 16 h. After the reaction was completed, the mixture was cooled to room temperature and then filtered. The solid was collected and dried to afford 1-acetyl-3-(triphenyl-lambda5-phosphanylidene)pyrrolidin-2-one (2.2 g, crude) as a white solid. LCMS (ESI, m/z): [M+H]⁺=388.1.

### Step 5. Synthesis of 3-[2-(dimethylamino)ethylidene]pyrrolidin-2-one

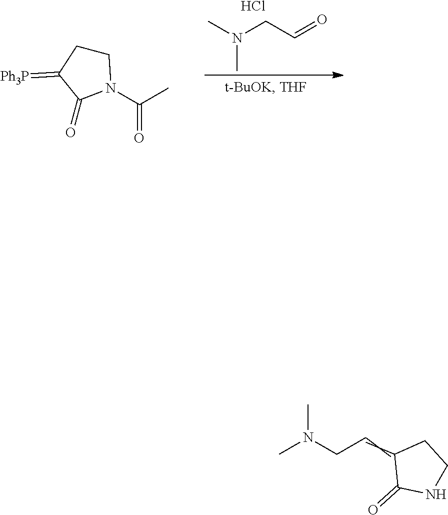

To a solution of 1-acetyl-3-(triphenyl-lambda5-phosphanylidene)pyrrolidin-2-one (2.2 g, crude) in THE (25.0 mL) was added t-BuOK (1.6 g, 14.61 mmol) at 0° C. under N₂. The mixture was stirred at 0° C. for 30 min. Then 2-(dimethylamino)acetaldehyde hydrochloride (1.3 g, 10.52 mmol) was added to the mixture. The mixture was stirred at room temperature for another 16 h. The mixture was quenched with saturated NH₄Cl solution and then extracted with EtOAc. The aqueous layer was purified by reverse phase flash column chromatography with CH₃CN/H₂O (1/99, v/v) to afford 3-[2-(dimethylamino)ethylidene]pyrrolidin-2-one (260.0 mg, crude) as a yellow semi-solid. LCMS (ESI, m/z): [M+H]⁺=155.1.

### Step 6. Synthesis of (Z)-3-[2-(dimethylamino)ethylidene]-1-[4-[(3-methyl-4-[[1,2,4]triazolo[1,5-a]pyridin-7-yloxy]phenyl)amino]quinazolin-6-yl]pyrrolidin-2-one and (E)-3-[2-(dimethylamino)ethylidene]-1-[4-[(3-methyl-4-[[1,2,4]triazolo[1,5-a]pyridin-7-yloxy]phenyl)amino]quinazolin-6-yl]pyrrolidin-2-one (Compounds 17 and 18)

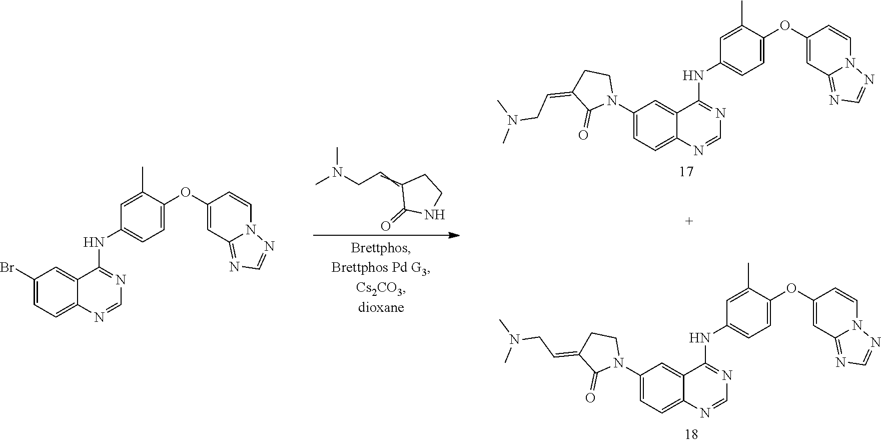 [SMILES: Cc1cc(Nc2ncnc3ccc(Br)cc23)ccc1Oc1ccn2ncnc2c1 ; CN(C)CC=C1CCNC1=O ; Cc1cc(Nc2ncnc3ccc(N4CC/C(=C/CN(C)C)C4=O)cc23)ccc1Oc1ccn2ncnc2c1 ; Cc1cc(Nc2ncnc3ccc(N4CC/C(=C\CN(C)C)C4=O)cc23)ccc1Oc1ccn2ncnc2c1]

A mixture of 3-[2-(dimethylamino)ethylidene]pyrrolidin-2-one (230.0 mg, crude), 6-bromo-N-(3-methyl-4-[[1,2,4]triazolo[1,5-a]pyridin-7-yloxy]phenyl)quinazolin-4-amine (180.0 mg, 0.40 mmol), BrettPhos (128.9 mg, 0.24 mmol), BrettPhos Pd G3 (112.4 mg, 0.12 mmol) and Cs₂CO₃ (443.1 mg, 1.36 mmol) in dioxane (10.0 mL) was stirred at 100° C. for 2 h. After the reaction was completed, the mixture was evaporated in vacuo. The residue was purified by flash column chromatography with CH₂Cl₂/CH₃OH (7/3, v/v) and then purified by Prep-HPLC with the following conditions: Column: XSelect CSH Prep C18 OBD Column, 19×250 mm, 5 um; Mobile Phase A: Water (0.05% FA), Mobile Phase B: MeOH Preparative; Flow rate: 25 mL/min; Gradient: 5% B to 25% B in 2 min; 25% B to 33% B in 10 min, 254 nm; RT1: 9.17 min; RT2: 10.05 min; to afford 3-[2-(dimethylamino)ethylidene]-1-[4-[(3-methyl-4-[[1,2,4]triazolo[1,5-a]pyridin-7-yloxy]phenyl)amino]quinazolin-6-yl]pyrrolidin-2-one Isomer 1 (retention time 9.17 min, 6.9 mg, 3%) as a white solid and 3-[2-(dimethylamino)ethylidene]-1-[4-[(3-methyl-4-[[1,2,4]triazolo[1,5-a]pyridin-7-yloxy]phenyl)amino]quinazolin-6-yl]pyrrolidin-2-one Isomer 2 (retention time 10.05 min, 0.6 mg, 0.3%) as a white solid. The absolute stereochemistry of Enantiomers 1 and 2 was not assigned. The two enantiomeric structures that could be obtained from chiral separation of the enantiomeric mixture as described above are shown as Compounds 17 and 18 in Table 1.

3-[2-(dimethylamino)ethylidene]-1-[4-[(3-methyl-4-[[1,2,4]triazolo[1,5-a]pyridin-7-yloxy]phenyl)amino]quinazolin-6-yl]pyrrolidin-2-one Isomer 1: LCMS (ESI, m/z): [M+H]⁺=521.2. ¹H NMR (300 MHz, CD₃OD): δ 8.75 (d, J=7.5 Hz, 1H), 8.57-8.50 (m, 2H), 8.40 (s, 1H), 8.30 (s, 1H), 7.84-7.74 (m, 3H), 7.20 (d, J=8.4 Hz, 1H), 7.09 (d, J=7.5 Hz, 1H), 6.83 (d, J=2.4 Hz, 1H), 6.69-6.59 (m, 1H), 4.20-4.10 (m, 2H), 3.55 (d, J=6.9 Hz, 2H), 3.07-2.97 (m, 2H), 2.61 (s, 6H), 2.26 (s, 3H).

3-[2-(dimethylamino)ethylidene]-1-[4-[(3-methyl-4-[[1,2,4]triazolo[1,5-a]pyridin-7-yloxy]phenyl)amino]quinazolin-6-yl]pyrrolidin-2-one Isomer 2: LCMS (ESI, m/z): [M+H]⁺=521.2. ¹H NMR (300 MHz, CD₃OD): δ 8.77 (d, J=7.5 Hz, 1H), 8.56-8.53 (m, 2H), 8.44 (d, J=2.1 Hz, 1H), 8.31 (s, 1H), 7.88-7.74 (m, 3H), 7.23 (d, J=8.7 Hz, 1H), 7.13-7.10 (m, 1H), 6.85 (d, J=2.4 Hz, 1H), 6.26-6.19 (m, 1H), 4.18-4.14 (m, 4H), 3.10-3.06 (m, 2H), 2.70 (s, 6H), 2.28 (s, 3H).

### Example S18: Synthesis of (E)-1-(4-(4-([1,2,4]triazolo[1,5-a]pyridin-7-yloxy)-3-methylphenylamino)quinazolin-6 yl)-3-(2-(dimethylamino)ethylidene)pyrrolidin-2-one hydrochloride (Compound 18)

### Step 1. Synthesis of tert-butyl 3-{2-[(tert-butoxycarbonyl)(methyl)amino]-1-hydroxyethyl}-2-oxopyrrolidine-1-carboxylate

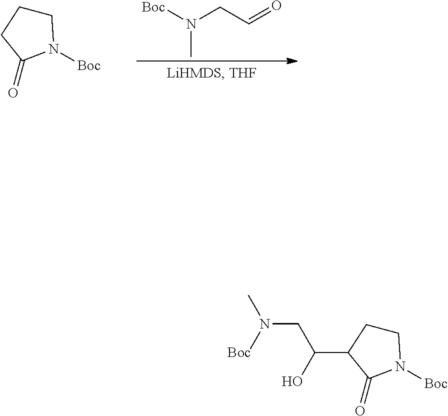 [SMILES: CC(C)(C)OC(=O)N1CCCC1=O ; CN(CC=O)C(=O)OC(C)(C)C ; CN(CC(O)C1CCN(C(=O)OC(C)(C)C)C1=O)C(=O)OC(C)(C)C]

To a solution of tert-butyl 2-oxopyrrolidine-1-carboxylate (5.7 g, 30.81 mmol) in THF (100.0 mL) was added dropwise LiHMDS (46.0 mL, 1 mol/L) at −78° C. under N₂. The mixture was stirred for at −78° C. for 1 h under N₂. Then a solution of tert-butyl N-methyl-N-(2-oxoethyl)carbamate (8.5 g, 49.13 mmol) in THF (20.0 mL) was added dropwise to the mixture at −78° C. The mixture was stirred at −78° C. for additional 1 h. After the reaction was completed, the reaction mixture was quenched with NH₄Cl (sat.) solution at −78° C. and then extracted with EtOAc. The combined organic layer was washed with brine, dried over anhydrous sodium sulfate and filtered. The filtrate was concentrated under vacuum to afford tert-butyl 3-{2-[(tert-butoxycarbonyl)(methyl)amino]-1-hydroxyethyl}-2-oxopyrrolidine-1-carboxylate (12.0 g, crude) as a light yellow oil. LCMS (ESI, m/z): [M+H]⁺=359.0.

### Step 2. Synthesis of tert-butyl 3-{2-[(tert-butoxycarbonyl)(methyl)amino]-1-(methanesulfonyloxy)ethyl}-2-oxopyrrolidine-1-carboxylate

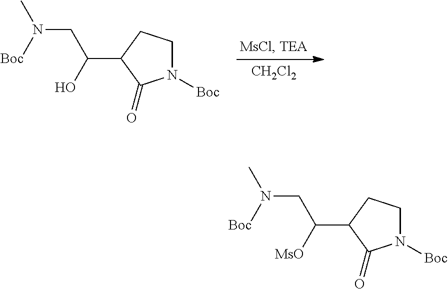

To a mixture of tert-butyl 3-{2-[(tert-butoxycarbonyl)(methyl)amino]-1-hydroxyethyl}-2-oxopyrrolidine-1-carboxylate (12.0 g, crude) and TEA (10.1 g, 100.43 mmol) in CH₂Cl₂ (100.0 mL) was added mesyl chloride (MsCl) (5.7 g, 50.21 mmol) at 0° C. The mixture was stirred at room temperature for 16 h. After the reaction was completed, the mixture was quenched with H₂O and extracted with CH₂Cl₂. The combined organic layer was washed with brine, dried over anhydrous sodium sulfate and filtered. The filtrate was concentrated under vacuum to tert-butyl 3-{2-[(tert-butoxycarbonyl)(methyl)amino]-1-(methanesulfonyloxy)ethyl}-2-oxopyrrolidine-1-carboxylate (15.0 g, crude) as a light yellow oil. LCMS (ESI, m/z): [M+H]⁺=437.0.

### Step 3. Synthesis of tert-butyl (E)-3-{2-[(tert-butoxycarbonyl)(methyl)amino]ethylidene}-2-oxopyrrolidine-1-carboxylate

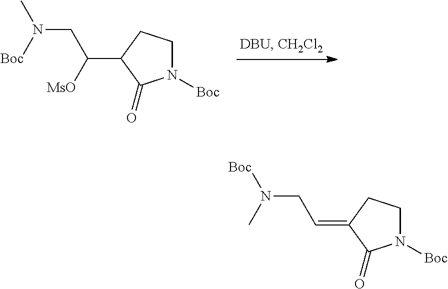 [SMILES: CN(CC(OS(C)(=O)=O)C1CCN(C(=O)OC(C)(C)C)C1=O)C(=O)OC(C)(C)C ; CN(C/C=C1\CCN(C(=O)OC(C)(C)C)C1=O)C(=O)OC(C)(C)C]

To a solution of tert-butyl 3-{2-[(tert-butoxycarbonyl)(methyl)amino]-1-(methanesulfonyloxy)ethyl}-2-oxopyrrolidine-1-carboxylate (15.0 g, crude) in CH₂Cl₂ (100.0 mL) was added DBU (6.3 g, 41.92 mmol) at 0° C. The mixture was stirred at room temperature for 3 h. After the reaction was completed, the mixture was diluted with H₂O and extracted with CH₂Cl₂. The combined organic layer was washed with brine, dried over anhydrous sodium sulfate and filtered. The filtrate was evaporated in vacuo. The residue was purified by flash column chromatography with petroleum ether/EtOAc (3/7, v/v) to afford tert-butyl (E)-3-{2-[(tert-butoxycarbonyl)(methyl)amino]ethylidene}-2-oxopyrrolidine-1-carboxylate (1.8 g, 15%) as a colorless oil. LCMS (ESI, m/z): [M+H]⁺=341.0.

### Step 4. Synthesis of (E)-3-[2-(methylamino)ethylidene]pyrrolidin-2-one trifluoroacetic acid

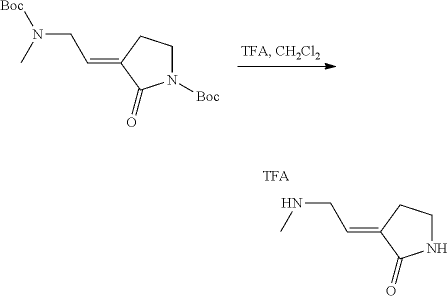

A mixture of tert-butyl (E)-3-{2-[(tert-butoxycarbonyl)(methyl)amino]ethylidene}-2-oxopyrrolidine-1-carboxylate (1.5 g, 4.40 mmol) and TFA (5.0 mL) in DCM (5.0 mL) was stirred at room temperature for 1 h. After the reaction was completed, the mixture was concentrated under reduced pressure to afford (E)-3-[2-(methylamino)ethylidene]pyrrolidin-2-one trifluoroacetic acid (2.3 g, crude) as a dark yellow oil. LCMS (ESI, m/z): [M+H]⁺=141.0.

### Step 5. Synthesis of (E)-3-[2-(dimethylamino)ethylidene]pyrrolidin-2-one

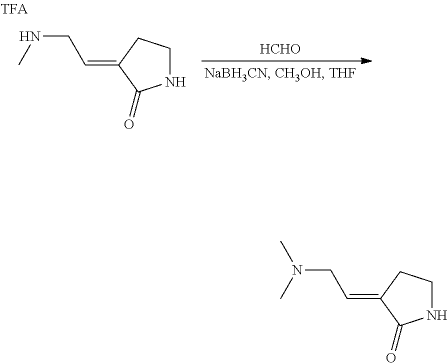

To a mixture of (E)-3-[2-(methylamino)ethylidene]pyrrolidin-2-one trifluoroacetic acid (2.3 g, crude) and HCHO (1.48 g, 49.22 mmol) in THF (10.0 mL)/CH₃OH (5.0 mL) was added NaBH₃CN (1.2 g, 20.20 mmol) at 0° C. The mixture was stirred at room temperature for 2 h. After the reaction was completed, the resulting mixture was concentrated under reduced pressure. The residue was purified by reverse phase flash chromatography with H₂O/CH₃OH (99/1, v/v) and purified by flash column chromatography with CH₂Cl₂/CH₃OH (4/1, v/v) to afford (E)-3-[2-(dimethylamino)ethylidene]pyrrolidin-2-one (150.0 mg, 6%) as a colorless oil. LCMS (ESI, m/z): [M+H]⁺=155.1.

### Step 6. Synthesis of (E)-1-(4-(4-([1,2,4]triazolo[1,5-a]pyridin-7-yloxy)-3-methylphenylamino)quinazolin-6-yl)-3-(2-(dimethylamino)ethylidene)pyrrolidin-2-one hydrochloride (Compound 18)

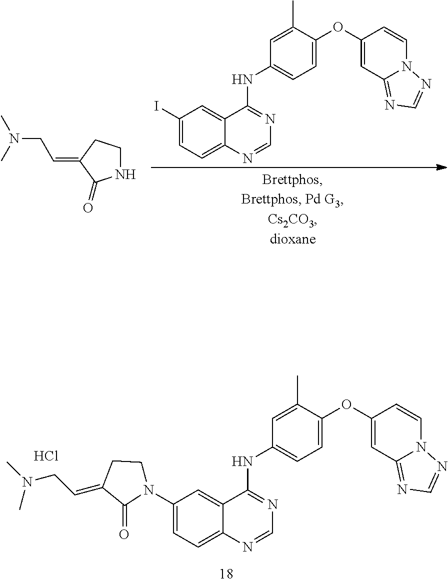 [SMILES: CN(C)C/C=C1\CCNC1=O ; Cc1cc(Nc2ncnc3ccc(I)cc23)ccc1Oc1ccn2ncnc2c1 ; Cc1cc(Nc2ncnc3ccc(N4CC/C(=C\CN(C)C)C4=O)cc23)ccc1Oc1ccn2ncnc2c1 ; Cl]

A mixture of (E)-3-[2-(dimethylamino)ethylidene]pyrrolidin-2-one (150.0 mg, 1.01 mmol), 6-iodo-N-(3-methyl-4-{[1,2,4]triazolo[1,5-a]pyridin-7-yloxy}phenyl)quinazolin-4-amine (200.0 mg, 0.40 mmol), BrettPhos (43.4 mg, 0.08 mmol), BrettPhos Pd G3 (36.6 mg, 0.01 mmol) and Cs₂CO₃ (395.5 mg, 1.21 mmol) in dioxane (5.0 mL) was stirred at 100° C. for 2 h. After the reaction was completed, the mixture was evaporated in vacuo. The residue was purified by flash column chromatography with CH₂Cl₂/MeOH (9/1, v/v) and then purified by Prep-HPLC with the following conditions (Column: Sunfire prep C18 column, 30×150 mm, 5 um; Mobile Phase A: ACN, Mobile Phase B: Water (0.05% HCl); Flow rate: 60 mL/min; Gradient: 5% B to 15% B in 12 min, 254 nm) to afford (E)-1-(4-(4-([1,2,4]triazolo[1,5-a]pyridin-7-yloxy)-3-methylphenylamino)quinazolin-6-yl)-3-(2-(dimethylamino)ethylidene)pyrrolidin-2-one hydrochloride (Compound 18) (21.7 mg, 9%) as a yellow solid. LCMS (ESI, m/z): [M+H]⁺=521.3. ¹H NMR (300 MHz, DMSO-d₆): δ 12.42 (s, 1H), 11.29 (s, 1H), 9.05-9.01 (m, 3H), 8.95 (s, 1H), 8.58 (s, 1H), 8.08 (d, J=9.3 Hz, 1H), 7.82 (d, J=2.1 Hz, 1H), 7.77-7.73 (m, 1H), 7.34-7.31 (m, 1H), 7.16-7.13 (m, 1H), 6.91 (d, J=2.1 Hz, 1H), 6.61-6.57 (m, 1H), 4.37-4.32 (m, 2H), 3.14-3.05 (m, 2H), 2.80 (s, 6H), 2.23 (s, 3H).

### Example S19: Synthesis of (R)-1-(4-((4-([1,2,4]triazolo[1,5-a]pyridin-7 yloxy)-3-methylphenyl)amino)quinazolin-6-yl)-4-methyl-3-methylenepyrrolidin-2-one (Compound 19)

### Step 1. Synthesis of tert-butyl (R)-4-methyl-2-oxopyrrolidine-1-carboxylate

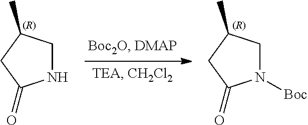

To a mixture of (R)-4-methylpyrrolidin-2-one (300.0 mg, 3.00 mmol) and TEA (0.9 g, 9.00 mmol) in CH₂Cl₂ (5.0 mL) was added Boc₂O (0.7 g, 3.00 mmol) and DMAP (7.4 mg, 0.06 mmol) at room temperature. The resulting mixture was stirred at room temperature for 16 h. After the reaction was completed, the mixture was concentrated under reduced pressure. The residue was purified by flash column chromatography with petroleum ether/EtOAc (7/3, v/v) to afford tert-butyl (R)-4-methyl-2-oxopyrrolidine-1-carboxylate (560.0 mg, 93%) as a colorless oil. LCMS (ESI, m/z): [M+H]⁺=200.0.

### Step 2. Synthesis of tert-butyl (R)-4-methyl-3-methylidene-2-oxopyrrolidine-1-carboxylate

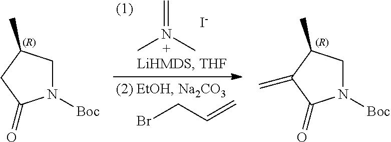

To a solution of tert-butyl (R)-4-methyl-2-oxopyrrolidine-1-carboxylate (560.0 mg, 2.81 mmol) in THF (15.0 mL) were added dropwise LiHMDS (4.2 mL, 1 mol/L) at −78° C. under N₂. The mixture was stirred at −78° C. for 1 h. Then Eschenmoser's salt (780.0 mg, 4.22 mmol) was added to the mixture at −78° C. The resulting mixture was stirred at −78° C. for additional 1 h. The mixture was warmed up to room temperature and quenched by the addition of sat. NH₄Cl (aq.). The resulting mixture was extracted with ethyl acetate. The combined organic layer was washed with brine, dried over anhydrous sodium sulfate and filtered. After filtration, the filtrate was concentrated under reduced pressure. The residue was diluted in EtOH (10.0 mL). Then allyl bromide (2.7 g, 22.48 mmol) and Na₂CO₃ (1.8 g, 16.86 mmol) were added to the mixture. The mixture was stirred at room temperature for 48 h. After the reaction was completed, the mixture was diluted with H₂O and extracted with EtOAc. The combined organic layer was washed with brine, dried over anhydrous sodium sulfate and filtered. The filtrate was evaporated in vacuo. The residue was purified by flash column chromatography with petroleum ether/EtOAc (45/55, v/v) to afford tert-butyl (R)-4-methyl-3-methylidene-2-oxopyrrolidine-1-carboxylate (320.0 mg, 53%) as a light yellow oil. LCMS (ESI, m/z): [M+H]⁺=212.1.

### Step 3. Synthesis of (R)-4-methyl-3-methylenepyrrolidin-2-one

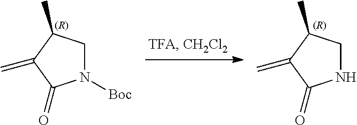

A mixture of tert-butyl (R)-4-methyl-3-methylidene-2-oxopyrrolidine-1-carboxylate (300.0 mg, 1.42 mmol) and TFA (3.0 mL) in THF (5.0 mL) was stirred at room temperature for 1 h. After the reaction was completed, the mixture was evaporated in vacuo to afford (R)-4-methyl-3-methylenepyrrolidin-2-one (130.0 mg, crude) as colorless oil. LCMS (ESI, m/z): [M+H]⁺=112.0.

### Step 4. Synthesis of 6-iodo-N-(3-methyl-4-[[1,2,4]triazolo[1,5-a]pyridin-7-yloxy]phenyl)quinazolin-4-amine

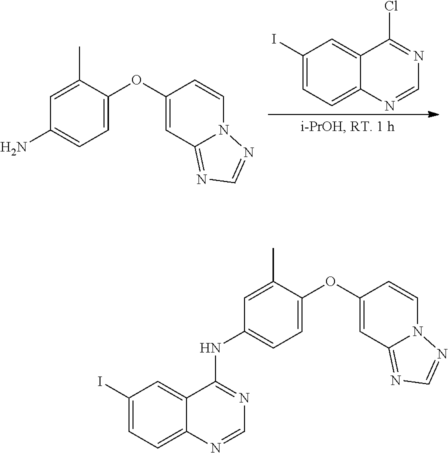 [SMILES: Cc1cc(N)ccc1Oc1ccn2ncnc2c1 ; Clc1ncnc2ccc(I)cc12 ; Cc1cc(Nc2ncnc3ccc(I)cc23)ccc1Oc1ccn2ncnc2c1]

A mixture of 3-methyl-4-[[1,2,4]triazolo[1,5-a]pyridin-7-yloxy]aniline (2.0 g, 8.32 mmol) and 4-chloro-6-iodoquinazoline (2.4 g, 8.33 mmol) in i-PrOH (40.0 mL) was stirred at room temperature for 1 h. After the reaction was completed, the mixture was concentrated under reduced pressure. The residue was purified by flash column chromatography with CH₂Cl₂/MeOH (9/1, v/v) to afford 6-iodo-N-(3-methyl-4-[[1,2,4]triazolo[1,5-a]pyridin-7-yloxy]phenyl)quinazolin-4-amine (1.6 g, 38%) as a yellow solid. LCMS (ESI, m/z): [M+H]⁺=495.0.

### Step 5. Synthesis of (R)-1-(4-((4-([1,2,4]triazolo[1,5-a]pyridin-7-yloxy)-3-methylphenyl)amino)quinazolin-6-yl)-4-methyl-3-methylenepyrrolidin-2-one (Compound 19)

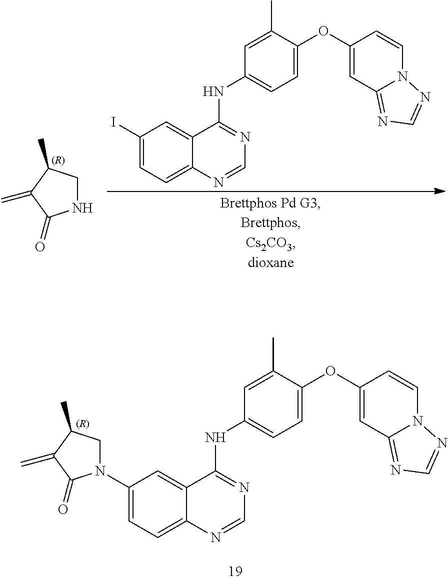

To a mixture of (R)-4-methyl-3-methylenepyrrolidin-2-one (130.0 mg, crude), 6-iodo-N-(3-methyl-4-[[1,2,4]triazolo[1,5-a]pyridin-7-yloxy]phenyl)quinazolin-4-amine (250.0 mg, 0.50 mmol) and Cs₂CO₃ (493.3 mg, 1.51 mmol) in dioxane (10.0 mL) were added BrettPhos (108.0 mg, 0.20 mmol) and BrettPhos Pd G3 (91.7 mg, 0.12 mmol) at room temperature under N₂. The mixture was stirred at 100° C. for 2 h. After the reaction was completed, the mixture was diluted with H₂O and extracted with EtOAc. The combined organic layer was washed with brine, dried over anhydrous sodium sulfate and filtered. The filtrate was evaporated in vacuo. The residue was purified by flash column chromatography with CH₂Cl₂/CH₃OH (92/8, v/v) and then purified by Prep-HPLC with the following conditions (Column: YMC-Actus Triart C18 ExRS, 30 mm×150 mm, 5 um; Mobile Phase A: Water (10 mmol/L NH₄HCO₃), Mobile Phase B: ACN; Flow rate: 60 mL/min; Gradient: 30% B to 50% B in 10 min; 254 nm) to afford (R)-1-(4-((4 ([1,2,4]triazolo[1,5-a]pyridin-7-yloxy)-3-methylphenyl)amino)quinazolin-6-yl)-4-methyl-3-methylenepyrrolidin-2-one (Compound 19) (103.1 mg, 43%) as a white solid. LCMS (ESI, m/z): [M+H]⁺=478.2. ¹H NMR (300 MHz, DMSO-d₆): δ 10.00-9.92 (m, 1H), 8.95 (d, J=7.5 Hz, 1H), 8.74-8.71 (m, 1H), 8.61 (d, J=6.0 Hz, 1H), 8.40 (s, 1H), 8.35 (s, 1H), 7.87-7.80 (m, 3H), 7.27-7.24 (m, 1H), 7.07-7.03 (m, 1H), 6.82 (d, J=2.4 Hz, 1H), 6.01 (d, J=2.7 Hz, 1H), 5.57 (d, J=2.1 Hz, 1H), 4.26-4.20 (m, 1H), 3.68-3.62 (m, 1H), 3.33-3.22 (m, 1H), 2.22 (s, 3H), 1.35 (d, J=6.6 Hz, 3H).

### Example S20: Synthesis of (4S)-4-methyl-1-{4-[(3-methyl-4-{[1,2,4]triazolo[1,5-a]pyridin-7-yloxy}phenyl)amino]quinazolin-6 yl}-3-methylidenepyrrolidin-2-one (Compound 20)

### Step 1. Synthesis of (S)-tert-butyl 4-methyl-2-oxopyrrolidine-1-carboxylate

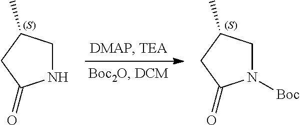

To a mixture of (S)-4-methylpyrrolidin-2-one (900.0 mg, 9.00 mmol) and TEA (2.7 g, 27.1 mmol) in CH₂Cl₂ (20.0 mL) was added Boc₂O (2.0 g, 9.17 mmol) and DMAP (22.1 mg, 0.18 mmol) at room temperature. The resulting mixture was stirred at room temperature for 16 h. After the reaction was completed, the mixture was concentrated under reduced pressure. The residue was purified by flash column chromatography with petroleum ether/EtOAc (7/3, v/v) to afford (S)-tert-butyl 4-methyl-2-oxopyrrolidine-1-carboxylate (1.4 g, 89%) as a colorless oil. LCMS (ESI, m/z): [M+H]⁺=200.0.

### Step 2. Synthesis of tert-butyl (S)-4-methyl-3-methylidene-2-oxopyrrolidine-1-carboxylate

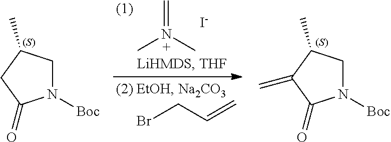

To a solution of tert-butyl (S)-4-methyl-2-oxopyrrolidine-1-carboxylate (1.4 g, 7.02 mmol) in THE (30.0 mL) were added dropwise LiHMDS (9.13 mL, 1 mol/L) at −78° C. under N₂. The mixture was stirred at −78° C. for 1 h. Then Eschenmoser's salt (1.95 g, 10.53 mmol) was added to the mixture at −78° C. The resulting mixture was stirred at −78° C. for additional 1 h. After the reaction was completed, the mixture was quenched with saturated NH₄Cl solution and then extracted with ethyl acetate. The combined organic layer was washed with brine, dried over anhydrous sodium sulfate and filtered. The filtrate was concentrated under reduced pressure. The residue was dissolved in EtOH (40.0 mL). Then allyl bromide (26.5 g, 219.19 mmol) and Na₂CO₃ (16.9 g, 160.07 mmol) were added to the mixture. The mixture was stirred at room temperature for 48 h. After the reaction was completed, the mixture was diluted with H₂O and extracted with EtOAc. The combined organic layer was washed with brine, dried over anhydrous sodium sulfate and filtered. The filtrate was evaporated in vacuo. The residue was purified by flash column chromatography with petroleum ether/EtOAc (45/55, v/v) to afford tert-butyl (S)-4-methyl-3-methylidene-2-oxopyrrolidine-1-carboxylate (470.0 mg, 31%) as a yellow oil. LCMS (ESI, m/z): [M+H]⁺=212.0.

### Step 3. Synthesis of (S)-4-methyl-3-methylenepyrrolidin-2-one

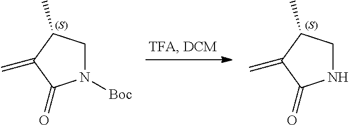

To a solution of tert-butyl (S)-4-methyl-3-methylidene-2-oxopyrrolidine-1-carboxylate (230.0 mg, 1.10 mmol) in CH₂Cl₂ (5.0 mL) was added TFA (2.5 mL) at room temperature. The resulting mixture was stirred at room temperature for 1 h. After the reaction was completed, the resulting mixture was concentrated under reduced pressure to afford (S)-4-methyl-3-methylenepyrrolidin-2-one (200.0 mg, crude) as a light yellow oil. LCMS (ESI, m/z): [M+H]⁺=112.0.

### Step 4. Synthesis of (S)-1-(4-(4-([1,2,4]triazolo[1,5-a]pyridin-7-yloxy)-3-methylphenylamino)quinazolin-6-yl)-4-methyl-3-methylenepyrrolidin-2-one (Compound 20)

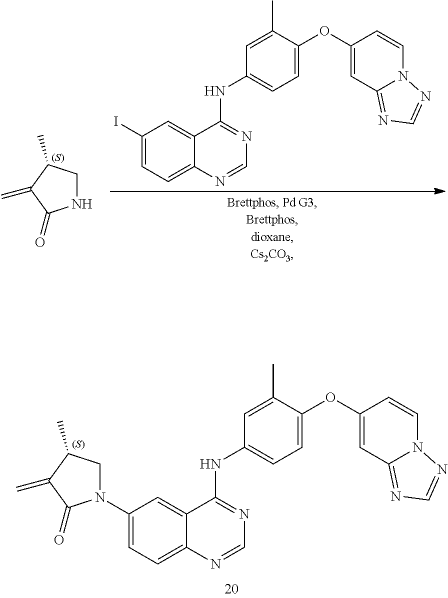

A mixture of (S)-4-methyl-3-methylidenepyrrolidin-2-one (200.0 mg, crude), 6-iodo-N-(3-methyl-4-{[1,2,4]triazolo[1,5-a]pyridin-7-yloxy}phenyl)quinazolin-4-amine (150.0 mg, 0.30 mmol), Cs₂CO₃ (593.2 mg, 1.82 mmol), BrettPhos Pd G3 (27.5 mg, 0.03 mmol) and BrettPhos (32.6 mg, 0.06 mmol) in 1,4-dioxane (6.0 mL) was stirred at 100° C. for 2 h under N₂. After the reaction was completed, the resulting mixture was concentrated under reduced pressure. The residue was purified by flash column chromatography with CH₂Cl₂/CH₃OH(7/3, v/v) and then purified by Prep-HPLC with the following conditions: (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 36% B to 45% B in 8 min; 254 nm) to afford (4S)-4-methyl-1-{4-[(3-methyl-4-{[1,2,4]triazolo[1,5-a]pyridin-7-yloxy}phenyl)amino]quinazolin-6-yl}-3-methylidenepyrrolidin-2-one (Compound 20) (26.4 mg, 18%) as a white solid. LCMS (ESI, m/z): [M+H]⁺=478.2. ¹H NMR (300 MHz, DMSO-d₆): δ 9.86 (s, 1H), 8.93 (d, J=7.5 Hz, 1H), 8.71-8.67 (m, 1H), 8.56 (s, 1H), 8.37 (s, 1H), 8.30 (d, J=2.4 Hz, 1H), 7.84-7.79 (m, 3H), 7.24-7.21 (m, 1H), 7.04-7.01 (m, 1H), 6.79 (d, J=2.4 Hz, 1H), 5.99 (d, J=2.7 Hz, 1H), 5.54 (d, J=2.4 Hz, 1H), 4.23-4.16 (m, 1H), 3.65-3.60 (m, 1H), 3.19-3.11 (m, 1H), 2.19 (s, 3H), 1.33 (d, J=6.6 Hz, 3H).

### Example S21: Synthesis of (1R,5R)-2-{4-[(3-methyl-4-{[1,2,4]triazolo[1,5-a]pyridin-7-yloxy}phenyl)amino]quinazolin-6-yl}-4-methylidene-2-azabicyclo[3.1.0]hexan-3-one and (1S,5S)-2-[4-{[(3-methyl-4-[[1,2,4]triazolo[1,5-a]pyridin-7-yloxy}phenyl)amino]quinazolin-6-yl}-4-methylidene-2-azabicyclo[3.1.0]hexan-3-one (Compounds 21 and 22)

### Step 1. Synthesis of tert-butyl 2-azabicyclo[3.1.0]hexane-2-carboxylate

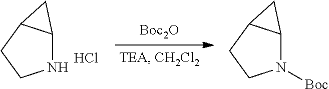 [SMILES: "Boc2O" ; C1CC2CC2N1 ; Cl ; CC(C)(C)OC(=O)N1CCC2CC21]

To a solution of 2-azabicyclo[3.1.0]hexane hydrochloride (2.0 g, 16.72 mmol) in CH₂Cl₂ (40.0 mL) was added TEA (4.2 g, 41.81 mmol) and Boc₂O (4.7 g, 21.74 mmol) at room temperature. The resulting mixture was stirred at room temperature for 16 h. After the reaction was completed, the reaction mixture was evaporated in vacuo. The residue was purified by flash column chromatography with petroleum ether/ethyl acetate (10/1, v/v) to afford tert-butyl 2-azabicyclo[3.1.0]hexane-2-carboxylate (2.0 g, 65%) as a colorless oil. LCMS (ESI, m/z): [M+H]⁺=184.1.

### Step 2. Synthesis of tert-butyl 3-oxo-2-azabicyclo[3.1.0]hexane-2-carboxylate

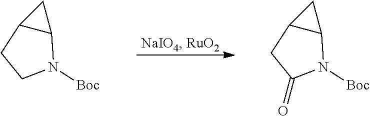

A mixture of ruthenium (iv) oxide hydrate (577.1 mg, 3.82 mmol) and NaIO₄ (1.2 g, 5.57 mmol) in H₂O (500.0 mL) was added dropwise to a solution of tert-butyl 2-azabicyclo[3.1.0]hexane-2-carboxylate (2.0 g, 10.91 mol) in EtOAc (650.0 mL). The resulting mixture was stirred at room temperature for 20 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/2, v/v) to afford tert-butyl 3-oxo-2-azabicyclo[3.1.0]hexane-2-carboxylate (2.2 g, 99%) as a light yellow solid. LCMS (ESI, m/z): [M+H]⁺=198.1.

### Step 3. Synthesis of tert-butyl 4-methylidene-3-oxo-2-azabicyclo[3.1.0]hexane-2-carboxylate

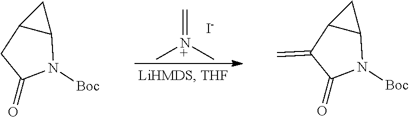

To a solution of tert-butyl 3-oxo-2-azabicyclo[3.1.0]hexane-2-carboxylate (1.5 g, 7.60 mmol) in THF (20.0 mL) was added dropwise LiHMDS (12.9 mL, 1 mol/L) at −78° C. under N₂. The mixture was stirred at −78° C. for 1 h. Then Eschenmoser's salt (2.1 g, 11.41 mmol) was added to the mixture at −78° C. The resulting mixture was stirred at −78° C. for additional 1 h. The mixture was warmed up to room temperature and quenched by the addition of sat. NH₄Cl (aq.). The resulting mixture was extracted with ethyl acetate. The combined organic layer was washed with brine, dried over anhydrous sodium sulfate and filtered. After filtration, the filtrate was concentrated under reduced pressure. The residue was diluted in EtOH (40.0 mL). Allyl bromide (7.3 g, 60.84 mmol) and Na₂CO₃ (4836.3 mg, 45.63 mmol) were added to the mixture. The mixture was stirred at room temperature for 48 h. After the reaction was completed, the mixture was diluted with H₂O and extracted with EtOAc. The combined organic layer was washed with brine, dried over anhydrous sodium sulfate and filtered. The filtrate was evaporated in vacuo. The residue was purified by flash column chromatography with petroleum ether/EtOAc (40/60, v/v) to afford tert-butyl 4-methylidene-3-oxo-2-azabicyclo[3.1.0]hexane-2-carboxylate (900.0 mg, 56%) as a white solid. LCMS (ESI, m/z): [M+H]⁺=210.1.

### Step 4. Synthesis of 4-methylidene-2-azabicyclo[3.1.0]hexan-3-one

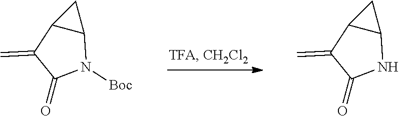

To a solution of tert-butyl 4-methylidene-3-oxo-2-azabicyclo[3.1.0]hexane-2-carboxylate (600.0 mg, 2.87 mmol) in CH₂Cl₂ (5 mL) was added TFA (1.0 mL) at room temperature. The resulting mixture was stirred at room temperature for 0.5 h. After the reaction was completed, the resulting mixture was concentrated under vacuum to afford 4-methylidene-2-azabicyclo[3.1.0]hexan-3-one (470.0 mg, crude) as a colorless oil. LCMS (ESI, m/z): [M+H]⁺=110.1.

### Step 5. Synthesis of 2-{4-[(3-methyl-4-{[1,2,4]triazolo[1,5-a]pyridin-7-yloxy}phenyl)amino]quinazolin-6-yl}-4-methylidene-2-azabicyclo[3.1.0]hexan-3-one

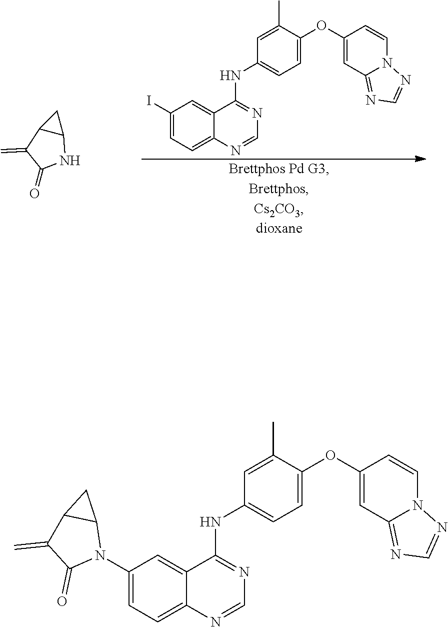

To a solution of 4-methylidene-2-azabicyclo[3.1.0]hexan-3-one (470.0 mg, crude) in dioxane (30.0 mL) was added 6-iodo-N-(3-methyl-4-{[1,2,4]triazolo[1,5-a]pyridin-7-yloxy}phenyl)quinazolin-4-amine (700.0 mg, 1.42 mmol), Cs₂CO₃ (1381.6 mg, 4.25 mmol), BrettPhos (152.2 mg, 0.29 mmol) and Brettphos Pd G3 (128.3 mg, 0.14 mmol) at room temperature under N₂. The resulting mixture was stirred at 100° C. for 2 h under N₂. After the reaction was completed, the resulting mixture was filtered. The filtrate was concentrated under reduced pressure. The residue was purified by flash chromatography with CH₂Cl₂/MeOH (10/1, v/v) to afford 2-{4-[(3-methyl-4-{[1,2,4]triazolo[1,5-a]pyridin-7-yloxy}phenyl)amino]quinazolin-6-yl-4-methylidene-2-azabicyclo[3.1.0]hexan-3-one (100.0 mg, 15%) as a white solid. LCMS (ESI, m/z): [M+H]⁺=476.2.

### Step 6. Synthesis of (1R,5R)-2-{4-[(3-methyl-4-{[1,2,4]triazolo[1,5-a]pyridin-7-yloxy}phenyl)amino]quinazolin-6-yl}-4-methylidene-2-azabicyclo[3.1.0]hexan-3-one and (1S,5S)-2-{4-[(3-methyl-4-{[1,2,4]triazolo[1,5-a]pyridin-7-yloxy}phenyl)amino]quinazolin-6-yl}-4-methylidene-2-azabicyclo[3.1.0]hexan-3-one (Compounds 21 and 22)

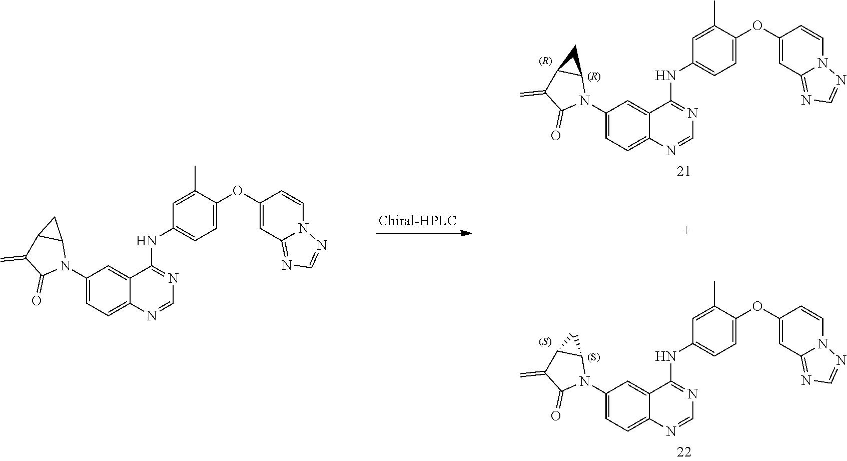

The racemic mixture of 2-{4-[(3-methyl-4-{[1,2,4]triazolo[1,5-a]pyridin-7-yloxy}phenyl)amino]quinazolin-6-yl}-4-methylidene-2-azabicyclo[3.1.0]hexan-3-one (100.0 mg, 0.21 mmol) was separated by Prep-Chiral-HPLC with the following conditions (Column: CHIRALPAK IH, 2×25 cm, 5 μm; Mobile Phase A: MeOH (0.5% 2M NH₃-MeOH)-HPLC, Mobile Phase B: EtOH-HPLC; Flow rate: 20 mL/min; Gradient: 70% B to 70% B in 16 min; 220/254 nm; RT1:12.169 min; RT2:14.244 min) to afford 2-{4-[(3-methyl-4-{[1,2,4]triazolo[1,5-a]pyridin-7-yloxy}phenyl)amino]quinazolin-6-yl}-4-methylidene-2-azabicyclo[3.1.0]hexan-3-one Enantiomer 1 (Retention time: 12.169 min, 26.1 mg, 25%) as a white solid and 2-{4-[(3-methyl-4-{[1,2,4]triazolo[1,5-a]pyridin-7-yloxy}phenyl)amino]quinazolin-6-yl}-4-methylidene-2-azabicyclo[3.1.0]hexan-3-one Enantiomer 2 (Retention time: 14.244 min, 21.1 mg, 20%) as a white solid. The absolute stereochemistry of Enantiomers 1 and 2 was not assigned. The two enantiomeric structures that could be obtained from chiral separation of the enantiomeric mixture as described above are shown as Compounds 21 and 22 in Table 1.

2-{4-[(3-methyl-4-{[1,2,4]triazolo[1,5-a]pyridin-7-yloxy}phenyl)amino]quinazolin-6-yl}-4-methylidene-2-azabicyclo[3.1.0]hexan-3-one Enantiomer 1: Retention time=12.169 min. LCMS (ESI, m/z): [M+H]⁺=476.1. ¹H NMR (400 MHz, DMSO-d₆): δ 9.95 (s, 1H), 8.95 (d, J=7.2 Hz, 1H), 8.61-8.59 (m, 2H), 8.47-8.44 (m, 1H), 8.39 (s, 1H), 7.88-7.79 (m, 3H), 7.24 (d, J=8.4 Hz, 1H), 7.06-7.03 (m, 1H), 6.81 (d, J=2.4 Hz, 1H), 6.01 (s, 1H), 5.75 (s, 1H), 4.08-4.05 (m, 1H), 2.58-2.52 (m, 1H), 2.21 (s, 3H), 1.47-1.45 (m, 1H), 0.99-0.96 (m, 1H).

2-{4-[(3-methyl-4-{[1,2,4]triazolo[1,5-a]pyridin-7-yloxy}phenyl)amino]quinazolin-6-yl}-4-methylidene-2-azabicyclo[3.1.0]hexan-3-one Enantiomer 2: Retention time 14.244 min. LCMS (ESI, m/z): [M+H]⁺=476.1. ¹H NMR (400 MHz, DMSO-d₆): δ 10.00 (s, 1H), 8.95 (d, J=7.6 Hz, 1H), 8.62-8.60 (m, 2H), 8.48-8.45 (m, 1H), 8.40 (s, 1H), 7.88-7.79 (m, 3H), 7.24 (d, J=8.4 Hz, 1H), 7.06-7.04 (m, 1H), 6.81 (d, J=2.4 Hz, 1H), 6.01 (s, 1H), 5.75 (s, 1H), 4.08-4.05 (m, 1H), 2.58-2.52 (m, 1H), 2.22 (s, 3H), 1.49-1.43 (m, 1H), 1.01-0.96 (m, 1H).

### Example S22: Synthesis of 1-{4-[(3-methyl-4-{[1,2,4]triazolo[1,5-a]pyridin-7-yloxy}phenyl)amino]pyrido[3,2-d]pyrimidin-6 yl}-3-methylidenepyrrolidin-2-one (Compound 23)

### Step 1. Synthesis of 6-chloro-N-(3-methyl-4-{[1,2,4]triazolo[1,5-a]pyridin-7-yloxy}phenyl)pyrido[3,2-d]pyrimidin-4-amine

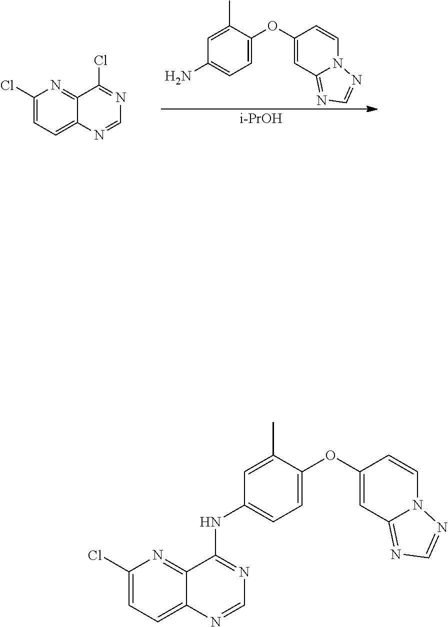 [SMILES: "i-PrOH" ; Clc1ccc2ncnc(Cl)c2n1 ; Cc1cc(N)ccc1Oc1ccn2ncnc2c1 ; Cc1cc(Nc2ncnc3ccc(Cl)nc23)ccc1Oc1ccn2ncnc2c1]

A mixture of 4,6-dichloropyrido[3,2-d]pyrimidine (500.0 mg, 2.50 mmol) and 3-methyl-4-[[1,2,4]triazolo[1,5-a]pyridin-7-yloxy]aniline (600.6 mg, 2.50 mmol) in i-PrOH (10.0 mL) was stirred at room temperature for 16 h. After the reaction was completed, the resulting mixture was concentrated under vacuum. The residue was purified by flash column chromatography with CH₂Cl₂/CH₃OH (94/6, v/v) to afford 6-chloro-N-(3-methyl-4-{[1,2,4]triazolo[1,5-a]pyridin-7-yloxy}phenyl)pyrido[3,2-d]pyrimidin-4-amine (220.0 mg, 21%) as a yellow solid. LCMS (ESI, m/z): [M+H]⁺=404.1.

### Step 2. Synthesis of 1-{4-[(3-methyl-4-{[1,2,4]triazolo[1,5-a]pyridin-7-yloxy}phenyl)amino]pyrido[3,2-d]pyrimidin-6-yl}-3-methylidenepyrrolidin-2-one (Compound 23)

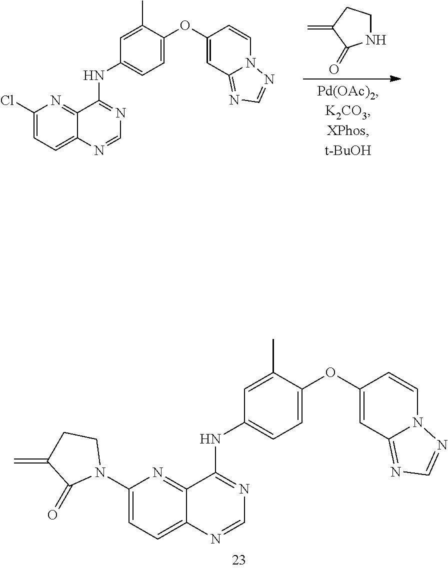

A mixture of 6-chloro-N-(3-methyl-4-{[1,2,4]triazolo[1,5-a]pyridin-7-yloxy}phenyl)pyrido[3,2-d]pyrimidin-4-amine (200.0 mg, 0.50 mmol), 3-methylidenepyrrolidin-2-one (144.3 mg, 1.49 mmol), K₂CO₃ (205.5 mg, 1.49 mmol), XPhos (47.2 mg, 0.10 mmol) and Pd(OAc)₂ (11.1 mg, 0.05 mmol) in t-BuOH (5.0 mL) was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) and then purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 32% B to 42% B in 9 min; 254/220 nm) to afford 1-{4-[(3-methyl-4-{[1,2,4]triazolo[1,5-a]pyridin-7-yloxy}phenyl)amino]pyrido[3,2-d]pyrimidin-6-yl}-3-methylidenepyrrolidin-2-one (Compound 23) (9.4 mg, 3%) as a yellow solid. LCMS (ESI, m/z): [M+H]⁺=465.2. ¹H NMR (300 MHz, DMSO-d₆): δ 9.67 (s, 1H), 9.01-8.94 (m, 2H), 8.66 (s, 1H), 8.39 (s, 1H), 8.32-8.29 (m, 1H), 8.01-7.97 (m, 2H), 7.27 (d, J=8.4 Hz, 1H), 7.06-7.03 (m, 1H), 6.81 (d, J=2.4 Hz, 1H), 6.09 (s, 1H), 5.64 (s, 1H), 4.38-4.33 (m, 2H), 2.99-2.94 (m, 2H), 2.14 (s, 3H).

### Example S23: Synthesis of 4-((4-([1,2,4]triazolo[1,5-a]pyridin-7 yloxy)-3-methylphenyl)amino)-6-(3-methylene-2-oxopyrrolidin-1 yl)quinoline-3-carbonitrile (Compound 24)

### Step 1. Synthesis of 4-((4-([1,2,4]triazolo[1,5-a]pyridin-7-yloxy)-3-methylphenyl)amino)-6-bromoquinoline-3-carbonitrile

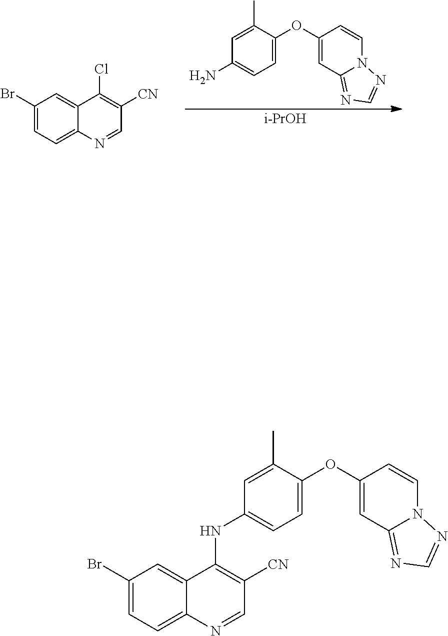 [SMILES: "i-PrOH" ; N#Cc1cnc2ccc(Br)cc2c1Cl ; Cc1cc(N)ccc1Oc1ccn2ncnc2c1 ; Cc1cc(Nc2c(C#N)cnc3ccc(Br)cc23)ccc1Oc1ccn2ncnc2c1]

To a solution of 6-bromo-4-chloroquinoline-3-carbonitrile (300 mg, 1.12 mmol) in propan-2-ol (10.0 mL) was added 3-methyl-4-[[1,2,4]triazolo[1,5-a]pyridin-7-yloxy]aniline (269.5 mg, 1.12 mmol) at 0° C. under N₂. The resulting mixture was stirred at room temperature for 16 h. After the reaction was completed, the mixture was concentrated under reduced pressure. The residue was purified by flash column chromatography with CH₂Cl₂/CH₃OH (10/1, v/v) to afford 4-((4-([1,2,4]triazolo[1,5-a]pyridin-7-yloxy)-3-methylphenyl)amino)-6-bromoquinoline-3-carbonitrile (320.0 mg, 60%) as a yellow solid. LCMS (ESI, m/z): [M+H]⁺=471.0.

### Step 2. Synthesis of 4-((4-([1,2,4]triazolo[1,5-a]pyridin-7-yloxy)-3-methylphenyl)amino)-6-(3-methylene-2-oxopyrrolidin-1-yl)quinoline-3-carbonitrile (Compound 24)

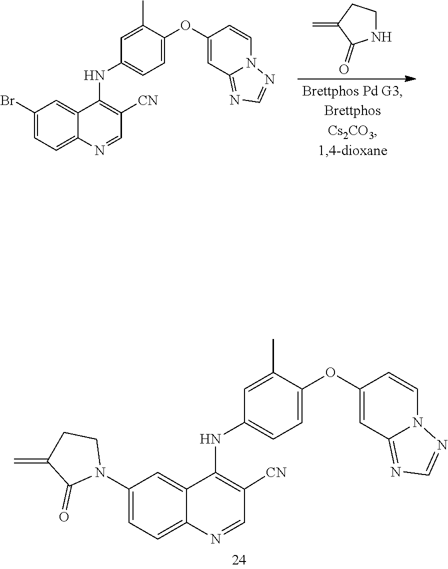

To a solution of 4-((4-([1,2,4]triazolo[1,5-a]pyridin-7-yloxy)-3-methylphenyl)amino)-6-bromoquinoline-3-carbonitrile (300.0 mg, 0.64 mmol) in 1,4-dioxane (10.0 mL) was added 3-methylenepyrrolidin-2-one (185.5 mg, 1.91 mmol), Cs₂CO₃ (622.2 mg, 1.91 mmol), Brettphos (68.3 mg, 0.13 mmol) and Brettphos Pd G3 (57.7 mg, 0.06 mmol) at room temperature under N₂. The resulting mixture was stirred at 100° C. for 2 h under N₂. After the reaction was completed, the mixture was concentrated under reduced pressure. The residue was purified by flash column chromatography with CH₂Cl₂/CH₃OH (10/1, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 30% to 43% in 8 min; 254 nm) to afford 4-((4-([1,2,4]triazolo[1,5-a]pyridin-7-yloxy)-3-methylphenyl)amino)-6-(3-methylene-2-oxopyrrolidin-1-yl)quinoline-3-carbonitrile (Compound 24) (48.0 mg, 15%) as a yellow solid. LCMS (ESI, m/z): [M+H]⁺=488.3. ¹H NMR (400 MHz, DMSO-d₆): δ 9.94 (s, 1H), 8.94 (d, J=7.2 Hz, 1H), 8.70 (d, J=9.2 Hz, 1H), 8.52 (d, J=8.8 Hz, 1H), 8.37-8.33 (m, 2H), 7.97 (d, J=9.2 Hz, 1H), 7.42 (s, 1H), 7.34-7.26 (m, 2H), 7.08-7.06 (m, 1H), 6.89 (d, J=2.0 Hz, 1H), 6.00 (s, 1H), 5.55 (s, 1H), 4.06-4.02 (m, 2H), 3.03-2.95 (m, 2H), 2.19 (s, 3H).

### Example S24: Synthesis of 1-(4-((4-([1,2,4]triazolo[1,5-a]pyridin-7 yloxy)-3-chlorophenyl)amino)-7-(2-(dimethylamino)ethoxy)quinazolin-6-yl)-3-methylenepyrrolidin-2-one (Compound 25)

### Step 1. Synthesis of 7-(2-chloro-4-nitrophenoxy)-[1,2,4]triazolo[1,5-a]pyridine

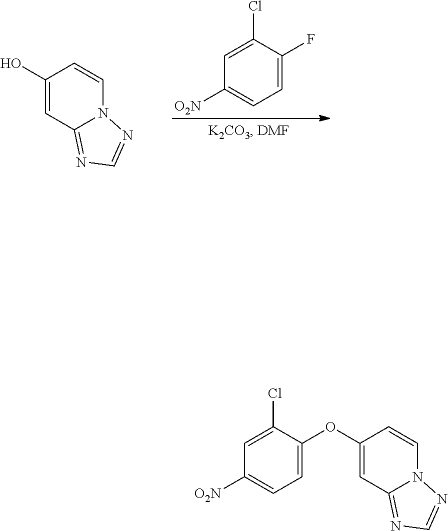 [SMILES: Oc1ccn2ncnc2c1 ; O=[N+]([O-])c1ccc(F)c(Cl)c1 ; O=[N+]([O-])c1ccc(Oc2ccn3ncnc3c2)c(Cl)c1]

To a solution of [1,2,4]triazolo[1,5-a]pyridin-7-ol (6.0 g, 44.40 mmol) in DMF (60.0 mL) was added K₂CO₃ (6.1 g, 44.40 mmol) and 2-chloro-1-fluoro-4-nitrobenzene (7.8 g, 44.40 mmol) at room temperature. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 7-(2-chloro-4-nitrophenoxy)-[1,2,4]triazolo[1,5-a]pyridine (4.0 g, 30%) as a white solid. LCMS (ESI, m/z): [M+H]+=291.0.

### Step 2. Synthesis of 3-chloro-4-{[1,2,4]triazolo[1,5-a]pyridin-7-yloxy}aniline

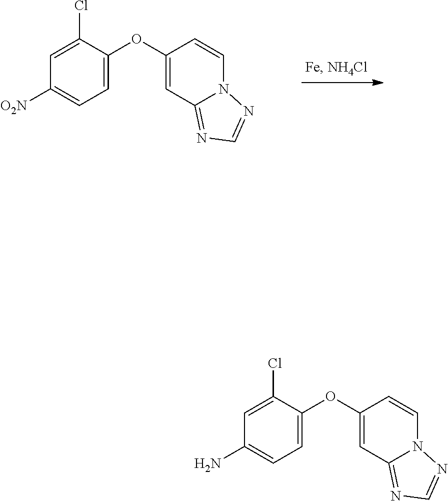 [SMILES: O=[N+]([O-])c1ccc(Oc2ccn3ncnc3c2)c(Cl)c1 ; Nc1ccc(Oc2ccn3ncnc3c2)c(Cl)c1]

To a mixture of 7-(2-chloro-4-nitrophenoxy)-[1,2,4]triazolo[1,5-a]pyridine (3.8 g, 13.07 mmol) and NH₄Cl (2.1 g, 39.22 mmol) in EtOH (10.0 mL)/H₂O (2.0 mL) was added Fe (2.2 g, 39.22 mmol) at 80° C. The resulting mixture was stirred at 80° C. for 3 h. After the reaction was completed, the resulting mixture was filtered. The filtrate was concentrated under reduced pressure. The residue was purified by flash column chromatography with CH₂Cl₂/MeOH (5/1, v/v) to afford 3-chloro-4-{[1,2,4]triazolo[1,5-a]pyridin-7-yloxy}aniline (2.9 g, 85%) as a white solid. LCMS (ESI, m/z): [M+H]⁺=261.0.

### Step 3. Synthesis of 6-bromo-N-(3-chloro-4-{[1,2,4]triazolo[1,5-a]pyridin-7-yloxy}phenyl)-7-methoxyquinazolin-4-amine

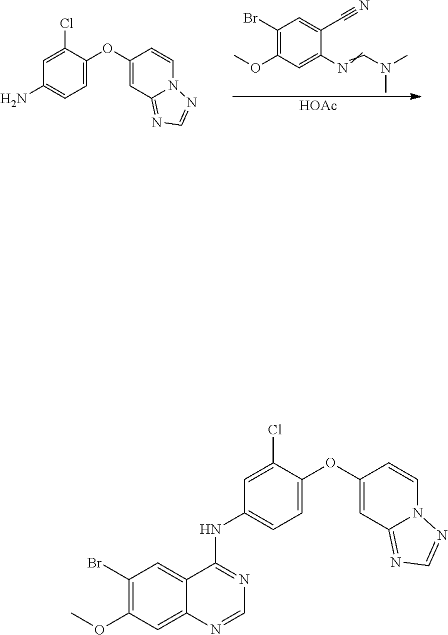

To a mixture of 3-chloro-4-{[1,2,4]triazolo[1,5-a]pyridin-7-yloxy}aniline (1.0 g, 3.87 mmol) in HOAc (150.0 mL) was added N′-(4-bromo-2-cyano-5-methoxyphenyl)-N,N-dimethylmethanimidamide (1.1 g, 3.87 mmol) at room temperature. The mixture was stirred at 85° C. for 4 h. After the reaction was completed, the resulting mixture was concentrated under reduced pressure. The residue was purified by flash column chromatography with petroleum CH₂Cl₂/MeOH (10/1, v/v) to afford 6-bromo-N-(3-chloro-4-{[1,2,4]triazolo[1,5-a]pyridin-7-yloxy}phenyl)-7-methoxyquinazolin-4-amine (1.2 g, 62%) as an off-white solid. LCMS (ESI, m/z): [M+H]⁺=497.0.

### Step 4. Synthesis of 6-bromo-4-[(3-chloro-4-{[1,2,4]triazolo[1,5-a]pyridin-7-yloxy}phenyl)amino]quinazolin-7-ol

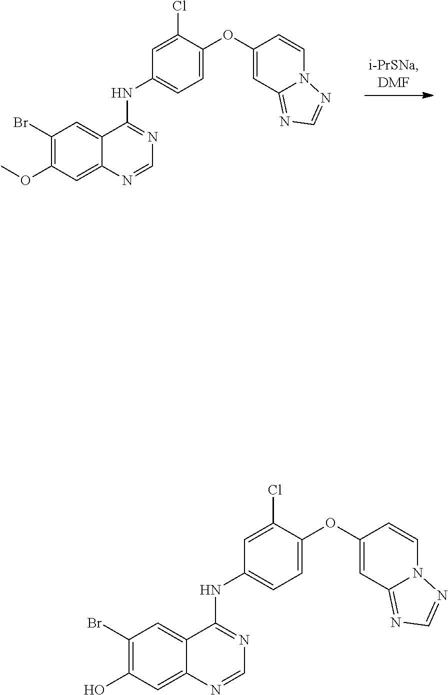 [SMILES: COc1cc2ncnc(Nc3ccc(Oc4ccn5ncnc5c4)c(Cl)c3)c2cc1Br ; Oc1cc2ncnc(Nc3ccc(Oc4ccn5ncnc5c4)c(Cl)c3)c2cc1Br]

To a solution of 6-bromo-N-(3-chloro-4-{[1,2,4]triazolo[1,5-a]pyridin-7-yloxy}phenyl)-7-methoxyquinazolin-4-amine (1.0 g, 2.01 mmol) in DMF (10.0 mL) was added i-PrSNa (1.1 g, 10.05 mmol) at room temperature. The resulting mixture was stirred at 135° C. for 30 min. After the reaction was completed, the resulting mixture was diluted with hydrochloric acid solution and then extracted with ethyl acetate. The organic layer was washed with brine, dried over anhydrous sodium sulfate and filtered. The filtrate was concentrated under vacuum to afford 6-bromo-4-[(3-chloro-4-{[1,2,4]triazolo[1,5-a]pyridin-7-yloxy}phenyl)amino]quinazolin-7-ol (400.0 mg, crude) as a yellow crude solid. LCMS (ESI, m/z): [M+H]⁺=483.0.

### Step 5. Synthesis of 6-bromo-N-(3-chloro-4-{[1,2,4]triazolo[1,5-a]pyridin-7-yloxy}phenyl)-7-[2-(dimethylamino)ethoxy]quinazolin-4-amine

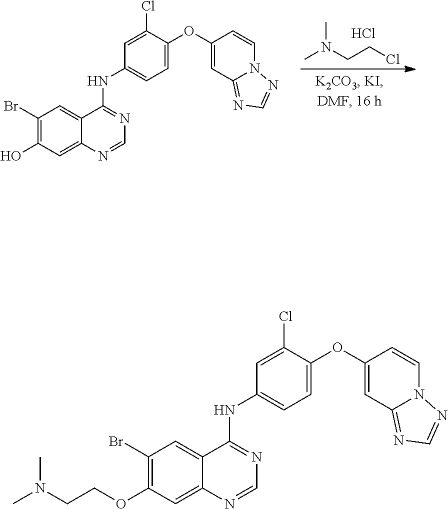

To a solution of 6-bromo-4-[(3-chloro-4-{[1,2,4]triazolo[1,5-a]pyridin-7-yloxy}phenyl)amino]quinazolin-7-ol (790.0 mg, crude) in DMF (10.0 mL) was added K₂CO₃ (902.8 mg, 6.53 mmol), dimethylaminoethyl chloride hydrochloride (234.7 mg, 1.63 mmol) and KI (3.4 mg, 0.02 mmol) at room temperature. The resulting mixture was stirred at 50° C. for 16 h. After the reaction was completed, the resulting mixture was concentrated under reduced pressure. The residue was purified by reverse phase flash chromatography with CH₂Cl₂/MeOH (10/1, v/v) to afford 6-bromo-N-(3-chloro-4-{[1,2,4]triazolo[1,5-a]pyridin-7-yloxy}phenyl)-7-[2-(dimethylamino)ethoxy]quinazolin-4-amine (450.0 mg, 49%) as a yellow solid. LCMS (ESI, m/z): [M+H]⁺=554.1.

### Step 6. Synthesis of 1-{4-[(3-chloro-4-{[1,2,4]triazolo[1,5-a]pyridin-7-yloxy}phenyl)amino]-7-[2-(dimethylamino)ethoxy]quinazolin-6-yl}-3-methylidenepyrrolidin-2-one (Compound 25)

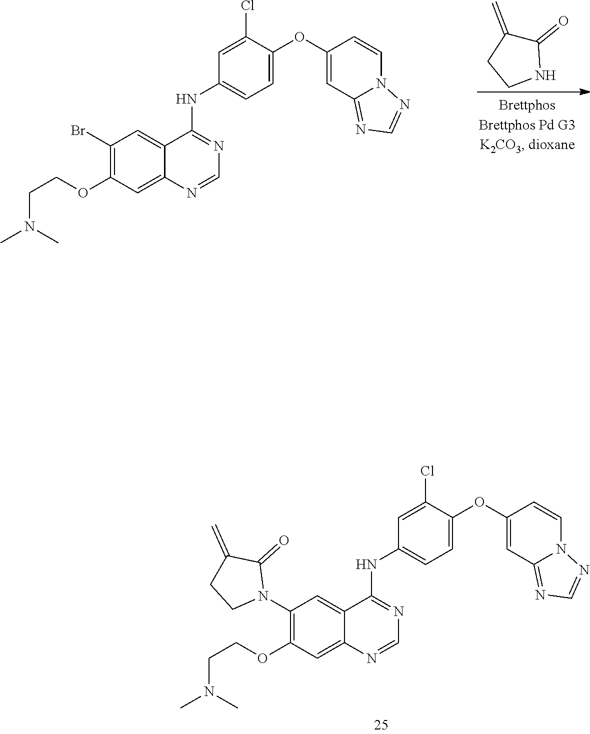

To a solution of 6-bromo-N-(3-chloro-4-{[1,2,4]triazolo[1,5-a]pyridin-7-yloxy}phenyl)-7-[2-(dimethylamino)ethoxy]quinazolin-4-amine (150.0 mg, 0.27 mmol) in dioxane (15.0 mL) was added 3-methylidenepyrrolidin-2-one (78.7 mg, 0.81 mmol), Cs₂CO₃ (704.7 mg, 2.16 mmol), Brettphos (29.0 mg, 0.05 mmol) and Brettphos Pd G3 (24.5 mg, 0.03 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 10% B to 20% B in 8 min, 20% B; Wave Length: 254 nm) to afford 1-{4-[(3-chloro-4-{[1,2,4]triazolo[1,5-a]pyridin-7-yloxy}phenyl)amino]-7-[2-(dimethylamino)ethoxy]quinazolin-6-yl}-3-methylidenepyrrolidin-2-one (Compound 25) (6.8 mg, 4%) as white solid. LCMS (ESI, m/z): [M+H]⁺=571.1. ¹H NMR (400 MHz, DMSO-d₆): δ 9.89 (s, 1H), 8.97 (d, J=7.6 Hz, 1H), 8.67 (s, 1H), 8.56 (s, 1H), 8.42 (s, 1H), 8.35 (d, J=2.4 Hz, 1H), 8.01-7.98 (m, 1H), 7.47 (d, J=8.8 Hz, 1H), 7.41 (s, 1H), 7.09-7.06 (m, 1H), 6.95 (d, J=2.0 Hz, 1H), 5.92 (s, 1H), 5.53 (s, 1H), 4.31-4.28 (m, 2H), 3.88-3.84 (m, 2H), 2.99-2.94 (m, 2H), 2.75-2.72 (m, 2H), 2.33-2.31 (m, 6H).

### Example S25: Synthesis of (E)-1-(4-((4-([1,2,4]triazolo[1,5-a]pyridin-7 yloxy)-3-methylphenyl)amino)quinazolin-6-yl)-3-(3-(dimethylamino)propylidene)pyrrolidin-2-one (Compound 26)

### Step 1. Synthesis of tert-butyl N-methyl-N-(3-oxopropyl)carbamate

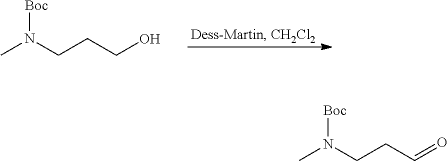

To a solution of tert-butyl N-(3-hydroxypropyl)-N-methylcarbamate (6.5 mmol) in DCM (30.0 mL) was added Dess-Martin (21.8 g, 51.51 mmol) at room temperature. The resulting mixture was stirred at room temperature for 2 h. After the reaction was completed, the mixture was diluted with petroleum ether and then filtered. The filtrate was concentrated under reduced pressure. The residue was purified by flash column chromatography with petroleum ether/ethyl acetate (4/6, v/v) to afford tert-butyl N-methyl-N-(3-oxopropyl)carbamate (3.0 g, 46%) as a light yellow oil.

### Step 2. Synthesis of tert-butyl 3-{3-[(tert-butoxycarbonyl)(methyl)amino]-1-hydroxypropyl}-2-oxopyrrolidine-1-carboxylate

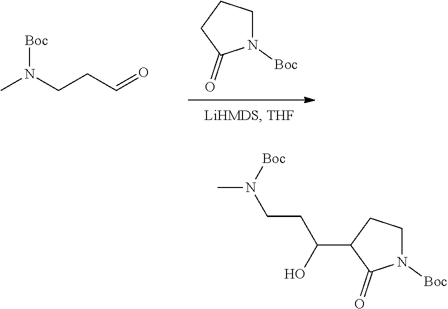 [SMILES: CN(CCC=O)C(=O)OC(C)(C)C ; CC(C)(C)OC(=O)N1CCCC1=O ; CN(CCC(O)C1CCN(C(=O)OC(C)(C)C)C1=O)C(=O)OC(C)(C)C]

To a solution of tert-butyl 2-oxopyrrolidine-1-carboxylate (3.0 g, 16.24 mmol) in THF (100.0 mL) was added LiHMDS (32.3 mL, 1 mol/L) at −78° C. under N₂. The mixture was stirred at −78° C. for 1 h under N₂. Then a solution of tert-butyl N-methyl-N-(3-oxopropyl)carbamate (3.0 g, 36.85 mmol) in THF (20.0 mL) was added dropwise to the mixture at −78° C. The mixture was stirred at −78° C. for additional 1 h. After the reaction was completed, the reaction mixture was quenched with NH₄Cl (sat.) solution and extracted with ethyl acetate. The combined organic layer was washed with brine, dried over anhydrous sodium sulfate and filtered. The filtrate was concentrated under vacuum to afford tert-butyl 3-{3-[(tert-butoxycarbonyl)(methyl)amino]-1-hydroxypropyl}-2-oxopyrrolidine-1-carboxylate (5.0 g, crude) as a light yellow oil. LCMS (ESI, m/z): [M+H]⁺=373.0.

### Step 3. Synthesis of tert-butyl 3-{3-[(tert-butoxycarbonyl)(methyl)amino]-1-(methanesulfonyloxy)propyl}-2-oxopyrrolidine-1-carboxylate

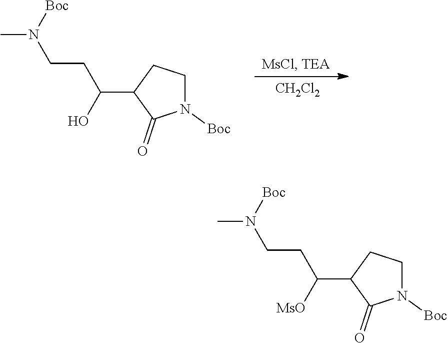 [SMILES: CN(CCC(O)C1CCN(C(=O)OC(C)(C)C)C1=O)C(=O)OC(C)(C)C ; "CH2Cl2" ; CN(CCC(OS(C)(=O)=O)C1CCN(C(=O)OC(C)(C)C)C1=O)C(=O)OC(C)(C)C]

To a mixture of tert-butyl 3-{3-[(tert-butoxycarbonyl)(methyl)amino]-1-hydroxypropyl}-2-oxopyrrolidine-1-carboxylate (5.0 g, crude) and TEA (2.7 g, 26.85 mmol) in CH₂Cl₂ (50.0 mL) was added MsCl (2.3 g, 20.13 mmol) at 0° C. The mixture was stirred at room temperature for 16 h. After the reaction was completed, the mixture was diluted with H₂O and extracted with CH₂Cl₂. The combined organic layer was washed with brine, dried over anhydrous sodium sulfate and filtered. The filtrate was concentrated under vacuum to tert-butyl 3-{3-[(tert-butoxycarbonyl)(methyl)amino]-1-(methanesulfonyloxy)propyl}-2-oxopyrrolidine-1-carboxylate (6.5 g, crude) as a light yellow oil. LCMS (ESI, m/z): [M+H]⁺=451.2.

### Step 4. Synthesis of (E)-tert-butyl 3-(3-(tert-butoxycarbonyl(methyl)amino)propylidene)-2-oxopyrrolidine-1-carboxylate

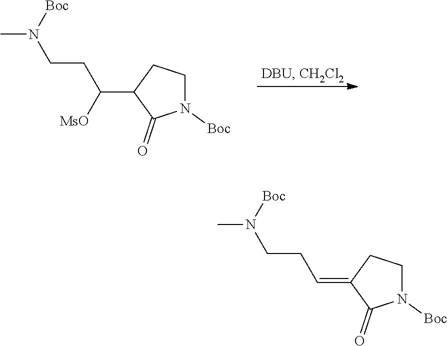 [SMILES: CN(CCC(OS(C)(=O)=O)C1CCN(C(=O)OC(C)(C)C)C1=O)C(=O)OC(C)(C)C ; CN(CC/C=C1\CCN(C(=O)OC(C)(C)C)C1=O)C(=O)OC(C)(C)C]

To a solution of tert-butyl 3-{3-[(tert-butoxycarbonyl)(methyl)amino]-1-(methanesulfonyloxy)propyl}-2-oxopyrrolidine-1-carboxylate (6.5 g, crude) in CH₂Cl₂ (80 mL) was added DBU (3.0 g, 20.19 mmol) at 0° C. The mixture was stirred at room temperature for 3 h. After the reaction was completed, the mixture was diluted with H₂O and extracted with CH₂Cl₂. The combined organic layer was washed with brine, dried over anhydrous sodium sulfate and filtered. The filtrate was evaporated in vacuo. The residue was purified by flash column chromatography with petroleum ether/EtOAc (3/7, v/v) to afford (E)-tert-butyl 3-(3-(tert-butoxycarbonyl(methyl)amino)propylidene)-2-oxopyrrolidine-1-carboxylate (1.2 g, 32%) as a colorless oil. LCMS (ESI, m/z): [M+H]⁺=355.0.

### Step 5. Synthesis of (E)-3-(3-(methylamino)propylidene)pyrrolidin-2-one trifluoroacetate

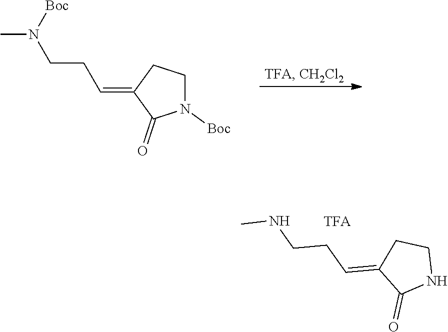 [SMILES: CN(CC/C=C1\CCN(C(=O)OC(C)(C)C)C1=O)C(=O)OC(C)(C)C ; CNCC/C=C1\CCNC1=O]

A mixture of tert-butyl (3E)-3-{3-[(tert-butoxycarbonyl)(methyl)amino]propylidene}-2-oxopyrrolidine-1-carboxylate (1.0 g, 2.82 mmol) and TFA (5.0 mL) in DCM (5.0 mL) was stirred at room temperature for 1 h. After the reaction was completed, the mixture was concentrated under reduced pressure to afford (E)-3-(3-(methylamino)propylidene)pyrrolidin-2-one trifluoroacetate (1.0 g, crude) as a dark yellow oil. LCMS (ESI, m/z): [M+H]⁺=155.0.

### Step 6. Synthesis of (3E)-3-[3-(dimethylamino)propylidene]pyrrolidin-2-one

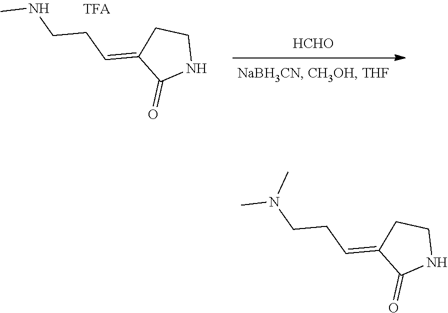

To a mixture of (3E)-3-[3-(methylamino)propylidene]pyrrolidin-2-one trifluoroacetate (1.0 g, crude) and HCHO (2.08 g, 49.22 mmol) in THF (10.0 mL)/CH₃OH (2.0 mL) was added NaBH₃CN (1.8 g, 29.18 mmol) at 0° C. The mixture was stirred at room temperature for 2 h. After the reaction was completed, the resulting mixture was concentrated under reduced pressure. The residue was purified by reverse phase flash chromatography with H₂O/CH₃OH (95/5, v/v) afford (3E)-3-[3-(dimethylamino)propylidene]pyrrolidin-2-one (600.0 mg, 55%) as a colorless oil. LCMS (ESI, m/z): [M+H]⁺=169.0.

### Step 7. Synthesis of (3E)-3-[3-(dimethylamino)propylidene]-1-{4-[(3-methyl-4-{[1,2,4]triazolo[1,5-a]pyridin-7-yloxy}phenyl)amino]quinazolin-6-yl}pyrrolidin-2-one (Compound 26)

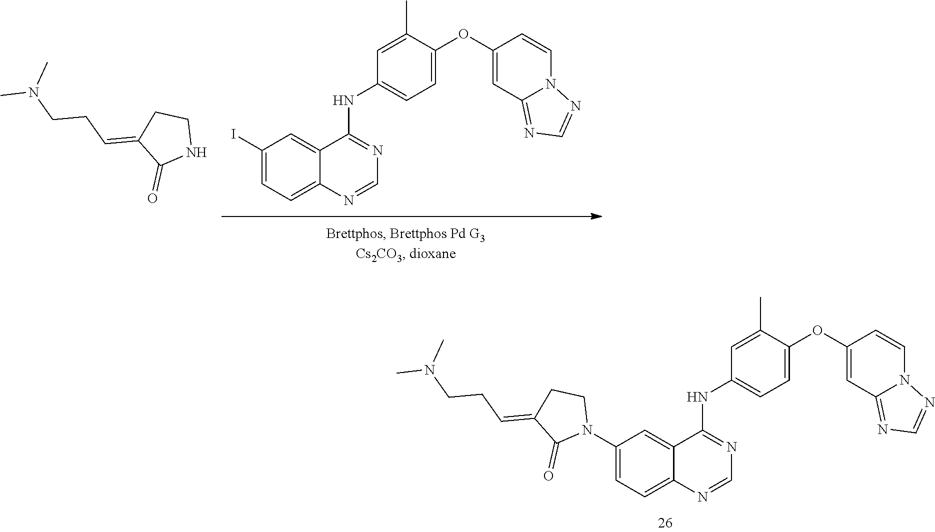

A mixture of (3E)-3-[3-(dimethylamino)propylidene]pyrrolidin-2-one (272.2 mg, 1.62 mmol), 6-iodo-N-(3-methyl-4-{[1,2,4]triazolo[1,5-a]pyridin-7-yloxy}phenyl)quinazolin-4-amine (200.0 mg, 0.40 mmol), BrettPhos (86.8 mg, 0.16 mmol), BrettPhos Pd G3 (73.3 mg, 0.08 mmol) and Cs₂CO₃ (395.5 mg, 1.21 mmol) in dioxane (8.0 mL) was stirred at 100° C. for 2 h. After the reaction was completed, the mixture was evaporated in vacuo. The residue was purified by flash column chromatography with CH₂Cl₂/MeOH (9/1, v/v) and then purified by Prep-HPLC with the following conditions (Column: XBridge Prep OBD C18 Column, 19×250 mm, 5 μm; Mobile Phase A: Water (10 mmol/L NH₄HCO₃), Mobile Phase B: MeOH; Flow rate: 25 mL/min; Gradient: 60% B to 65% B in 10 min, 254 nm) to afford (3E)-3-[3-(dimethylamino)propylidene]-1-{4-[(3-methyl-4-{[1,2,4]triazolo[1,5-a]pyridin-7-yloxy}phenyl)amino]quinazolin-6-yl}pyrrolidin-2-one (Compound 26) (11.4 mg, 5.27%) as a white solid. LCMS (ESI, m/z): [M+H]⁺=535.4. ¹H NMR (400 MHz, DMSO-d₆): δ 9.85 (s, 1H), 8.95 (d, J=7.2 Hz, 1H), 8.75-8.72 (m, 1H), 8.58 (s, 1H), 8.39 (s, 1H), 8.31 (d, J=2.0 Hz, 1H), 7.85-7.80 (m, 3H), 7.25-7.23 (m, 1H), 7.06-7.03 (m, 1H), 6.82 (d, J=2.4 Hz, 1H), 6.53-6.49 (m, 1H), 4.09-4.05 (m, 2H), 2.91-2.87 (m, 2H), 2.43-2.32 (m, 4H), 2.23-2.13 (m, 9H).

### Example S26: 1-[4-({3-methyl-4 [(1-methyl-1,3-benzodiazol-5-yl)oxy]phenyl}amino)quinazolin-6-yl]-3-methylidenepyrrolidin-2-one (Compound 27)

### Step 1. Synthesis of 1-methyl-5-(2-methyl-4-nitrophenoxy)-1,3-benzodiazole

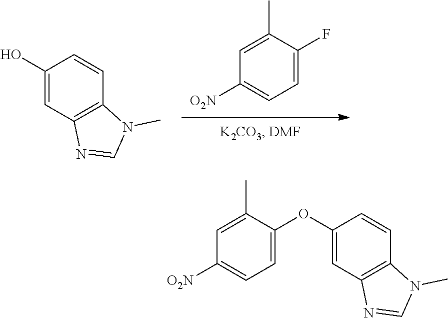 [SMILES: Cn1cnc2cc(O)ccc21 ; Cc1cc([N+](=O)[O-])ccc1F ; Cc1cc([N+](=O)[O-])ccc1Oc1ccc2c(c1)ncn2C]

To a solution of 1-methyl-1,3-benzodiazol-5-ol (2.0 g, 13.49 mmol) in DMF (50.0 mL) was added 1-fluoro-2-methyl-4-nitrobenzene (2.5 g, 16.19 mmol) and K₂CO₃ (3.7 g, 26.99 mmol) at room temperature. The resulting mixture was stirred at 50° C. for 16 h. After the reaction was completed, the mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with ethyl acetate/petroleum ether (50/50, v/v) to afford 1-methyl-5-(2-methyl-4-nitrophenoxy)-1,3-benzodiazole (3.8 g, 99%) as an off-white solid. LCMS (ESI, m/z): [M+H]⁺=284.1.

### Step 2. Synthesis of 3-methyl-4-[(1-methyl-1,3-benzodiazol-5-yl)oxy]aniline

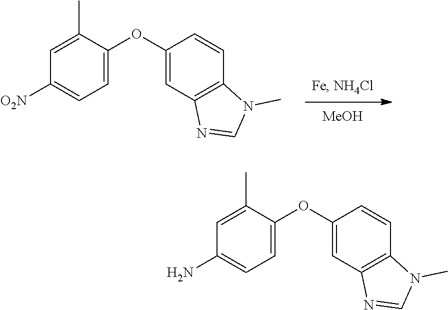

To a solution of 1-methyl-5-(2-methyl-4-nitrophenoxy)-1,3-benzodiazole (1.5 g, 5.29 mmol) in methanol (24.0 mL) and H₂O (8.0 mL) was added Fe (2.9 g, 52.95 mmol) and NH₄Cl (1.4 g, 26.50 mmol) at room temperature. The mixture was stirred at 80° C. for 1 h. After the reaction was completed, the resulting mixture was filtered. The filtrate was concentrated under vacuum. The residue was purified by flash column chromatography with CH₂Cl₂/MeOH (10/1, v/v) to afford 3-methyl-4-[(1-methyl-1,3-benzodiazol-5-yl)oxy]aniline (930.0 mg, 69%) as a yellow solid. LCMS (ESI, m/z): [M+H]⁺=254.1.

### Step 3. Synthesis of 6-iodo-N-{3-methyl-4-[(1-methyl-1,3-benzodiazol-5-yl)oxy]phenyl}quinazolin-4-amine

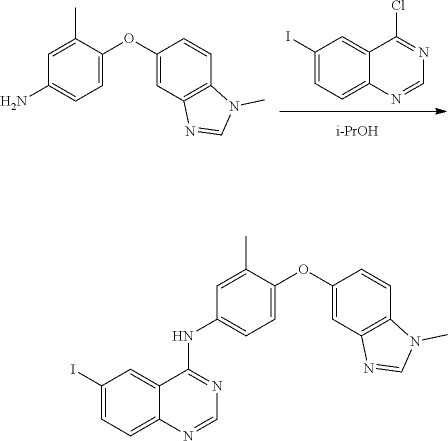 [SMILES: "i-PrOH" ; Cc1cc(N)ccc1Oc1ccc2c(c1)ncn2C ; Clc1ncnc2ccc(I)cc12 ; Cc1cc(Nc2ncnc3ccc(I)cc23)ccc1Oc1ccc2c(c1)ncn2C]

To a solution of 3-methyl-4-[(1-methyl-1,3-benzodiazol-5-yl)oxy]aniline (320.0 mg, 1.26 mmol) in i-PrOH (8.0 mL) was added 4-chloro-6-iodoquinazoline (403.6 mg, 1.38 mmol) at room temperature. The resulting mixture was stirred at 80° C. for 30 min. After the reaction was completed, the resulting mixture was concentrated under vacuum. The residue was purified by flash column chromatography with CH₂Cl₂/MeOH (10/1, v/v) to afford 6-iodo-N-{3-methyl-4-[(1-methyl-1,3-benzodiazol-5-yl)oxy]phenyl}quinazolin-4-amine (200.0 mg, 31%) as a yellow solid. LCMS (ESI, m/z): [M+H]⁺=508.1.

### Step 4. Synthesis of 1-[4-({3-methyl-4-[(1-methyl-1,3-benzodiazol-5-yl)oxy]phenyl}amino)quinazolin-6-yl]-3-methylidenepyrrolidin-2-one (Compound 27)

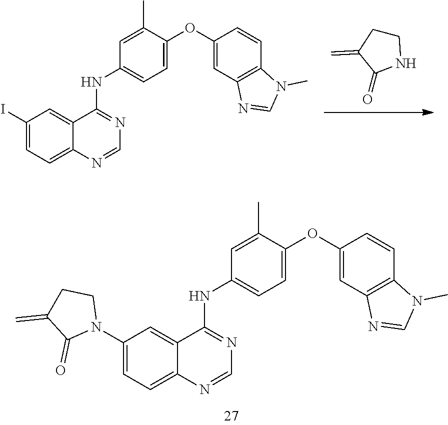

To a solution of 6-iodo-N-{3-methyl-4-[(1-methyl-1,3-benzodiazol-5-yl)oxy]phenyl}quinazolin-4-amine (170.0 mg, 0.33 mmol) in dioxane (4.0 mL) was added 3-methylidenepyrrolidin-2-one (97.6 mg, crude), BrettPhos Pd G3 (60.7 mg, 0.067 mmol), BrettPhos (71.9 mg, 0.13 mmol) and Cs₂CO₃ (327.5 mg, 1.00 mmol) at room temperature. The resulting mixture was stirred at 100° C. for 1 h under N₂. After the reaction was completed, the resulting mixture was filtered. The filtrate was concentrated under vacuum. The residue was purified by flash column chromatography with CH₂Cl₂/MeOH (10/1, v/v) and then purified by Prep-HPLC with the following conditions Column (XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 33% B to 43% B in 8 min; Wave Length: 254 nm) to afford 1-[4-({3-methyl-4-[(1-methyl-1,3-benzodiazol-5-yl)oxy]phenyl}amino)quinazolin-6-yl]-3-methylidenepyrrolidin-2-one (Compound 27) (31.6 mg, 19%) as a white solid. LCMS (ESI, m/z): [M+H]⁺=477.2. ¹H NMR (300 MHz, DMSO-d₆): δ 9.80 (s, 1H), 8.72-8.69 (m, 1H), 8.55 (s, 1H), 8.34 (s, 1H), 8.19 (s, 1H), 7.83 (d, J=9.0 Hz, 1H), 7.70 (s, 1H), 7.62-7.57 (m, 2H), 7.13 (s, 1H), 7.02 (d, J=8.7 Hz, 1H), 6.90 (d, J=9.0 Hz, 1H), 6.00 (s, 1H), 5.56 (s, 1H), 4.09-4.04 (m, 2H), 3.85 (s, 3H), 3.03-2.95 (m, 2H), 2.28 (s, 3H).

### Example S27: 1-(4-((4-([1,2,4]triazolo[1,5-a]pyridin-7 yloxy)-3-methylphenyl)amino)pyrido[3,4-d]pyrimidin-6-yl)-3-methylenepyrrolidin-2-one (Compound 28)

### Step 1. Synthesis of N-(4-([1,2,4]triazolo[1,5-a]pyridin-7-yloxy)-3-methylphenyl)-6-chloropyrido[3,4-d]pyrimidin-4-amine

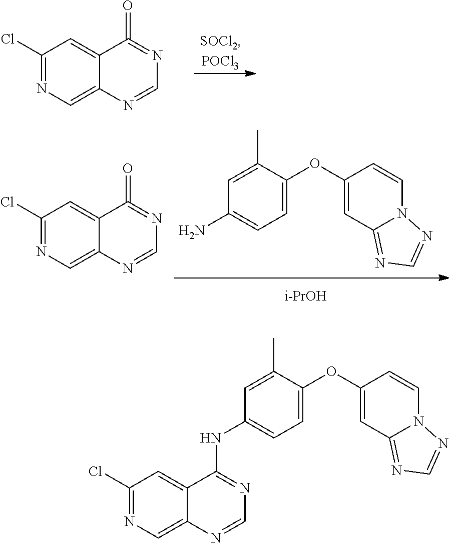

To a mixture of 6-chloro-3H-pyrido[3,4-d]pyrimidin-4-one (113.0 mg, 0.62 mmol) in SOCl₂ (2.0 mL) were added POCl₃ (0.7 mL) and DMF (0.1 mL) at room temperature. The resulting mixture was stirred at 85° C. for 2 h. After the reaction was completed, the resulting mixture was concentrated under reduced pressure. To the above residue in i-PrOH (5.0 mL) was added 4-([1,2,4]triazolo[1,5-a]pyridin-7-yloxy)-3-methylaniline (124.9 mg, 0.62 mmol) at room temperature. The resulting mixture was stirred at 25° C. for 16 h. After the reaction was completed, the resulting mixture was concentrated under reduced pressure. The residue was purified by flash column chromatography with CH₂Cl₂/CH₃OH (10/1, v/v) to afford 6-chloro-N-(3-methyl-4-{[1,2,4]triazolo[1,5-a]pyridin-7-yloxy}phenyl)pyrido[3,4-d]pyrimidin-4-amine (200.0 mg, 79%) as a brown yellow solid. LCMS (ESI, m/z): [M+H]⁺=404.1.

### Step 2. Synthesis of 1-(4-((4-([1,2,4]triazolo[1,5-a]pyridin-7-yloxy)-3-methylphenyl)amino)pyrido[3,4-d]pyrimidin-6-yl)-3-methylenepyrrolidin-2-one (Compound 28)

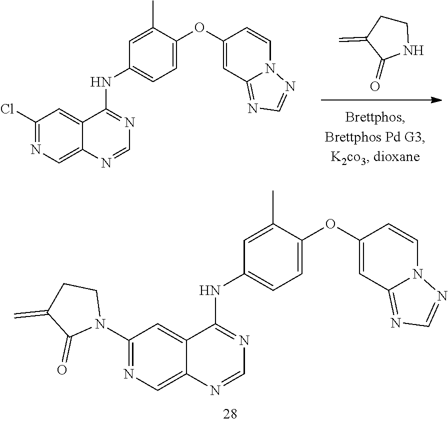

To a mixture of 6-chloro-N-(3-methyl-4-{[1,2,4]triazolo[1,5-a]pyridin-7-yloxy}phenyl)pyrido[3,4-d]pyrimidin-4-amine (200.0 mg, 0.50 mmol) and 3-methylidenepyrrolidin-2-one (90.0 mg, 0.97 mmol) in dioxane (10.0 mL) were added BrettPhos (53.2 mg, 0.10 mmol) and BrettPhos Pd G3 (44.9 mg, 0.05 mmol) and Cs₂CO₃ (484.1 mg, 1.49 mmol) at room temperature. The resulting mixture was stirred at 100° C. for 2 h. After the reaction was completed, the resulting mixture was filtered. The filtrate was concentrated under reduced pressure. The residue was purified by flash column chromatography with CH₂Cl₂/CH₃OH (17/3, v/v) and then purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 25% B to 50% B in 8 min; Wave Length: 254 nm) to afford 1-{4-[(3-methyl-4-{[1,2,4]triazolo[1,5-a]pyridin-7-yloxy}phenyl)amino]pyrido[3,4-d]pyrimidin-6-yl}-3-methylidenepyrrolidin-2-one (Compound 28) (26.3 mg, 11%) as a white solid. LCMS (ESI, m/z): [M+H]⁺=465.2. ¹H NMR (400 MHz, DMSO-d₆): δ 10.37 (s, 1H), 9.21 (s, 1H), 9.09 (s, 1H), 8.95 (d, J=7.6 Hz, 1H), 8.64 (s, 1H), 8.39 (s, 1H), 7.85-7.81 (m, 2H), 7.23 (d, J=8.4 Hz, 1H), 7.06-7.03 (m, 1H), 6.83 (d, J=2.4 Hz, 1H), 6.05 (s, 1H), 5.60 (s, 1H), 4.18-4.14 (m, 2H), 2.96-2.93 (m, 2H), 2.21 (s, 3H).

### Example S28: Synthesis of 4-(hydroxymethyl)-1-{4-[(3-methyl-4-[[1,2,4]triazolo[1,5-a]pyridin-7-yloxy}phenyl)amino]quinazolin-6-yl}-3-methylidenepyrrolidin-2-one (Compound 29)

### Step 1. Synthesis of 4-{[(tert-butyldiphenylsilyl)oxy]methyl}pyrrolidin-2-one

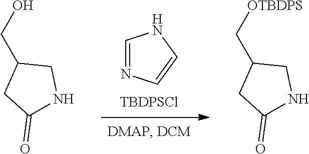

To a mixture of 4-(hydroxymethyl)pyrrolidin-2-one (2.0 g, 17.37 mmol) in DCM (100.0 mL) was added imidazole (2.9 g, 42.56 mmol), DMAP (0.3 g, 2.43 mmol) and tert-butyl(chloro)diphenylsilane (10.3 g, 37.35 mmol) at 0° C. The resulting mixture was stirred at room temperature for 16 h. After the reaction was completed, the reaction mixture was evaporated in vacuo. The residue was purified by flash column chromatography with DCM/MeOH (7/3, v/v) to afford 4-{[(tert-butyldiphenylsilyl)oxy]methyl}pyrrolidin-2-one (5.3 g, 86%) as a colorless oil. LCMS (ESI, m/z): [M+H]⁺=354.2.

### Step 2. Synthesis of tert-butyl 4-{[(tert-butyldiphenylsilyl)oxy]methyl}-2-oxopyrrolidine-1-carboxylate

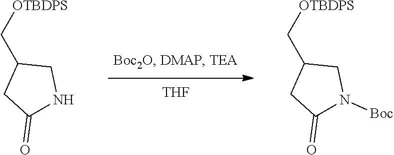

To a solution of 4-{[(tert-butyldiphenylsilyl)oxy]methyl}pyrrolidin-2-one (5.3 g, 14.99 mmol) in THF (100.0 mL) was added TEA (3.0 g, 29.98 mmol), DMAP (0.3 g, 2.99 mmol) and Boc₂O (3.9 g, 17.99 mmol) at room temperature. The resulting mixture was stirred at room temperature for 16 h. After the reaction was completed, the mixture was evaporated in vacuo. The residue was purified by flash column chromatography with petroleum ether/ethyl acetate (3/7, v/v) to afford tert-butyl 4-{[(tert-butyldiphenylsilyl)oxy]methyl}-2-oxopyrrolidine-1-carboxylate (6.5 g, 95%) as a colorless oil. LCMS (ESI, m/z): [M+H]⁺=454.2.

### Step 3. Synthesis of tert-butyl 4-{[(tert-butyldiphenylsilyl)oxy]methyl}-3-methylidene-2-oxopyrrolidine-1-carboxylate

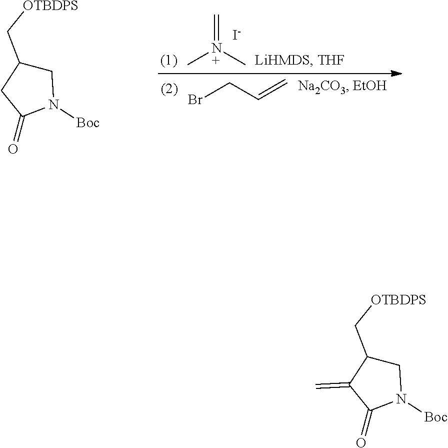

To a solution of tert-butyl 4-{[(tert-butyldiphenylsilyl)oxy]methyl}-2-oxopyrrolidine-1-carboxylate (6.5 g, 14.33 mmol) in THF (100.0 mL) was added dropwise LiHMDS (24.4 mL, 1 mol/L) at −78° C. under N₂. The mixture was stirred at −78° C. for 1 h. Then eschenmoser's salt (3.9 g, 21.49 mmol) was added to the mixture at −78° C. The resulting mixture was stirred at 78° C. for 1 h. After the reaction was completed, the mixture was quenched by the addition of sat. NH₄Cl(aq.). The resulting mixture was extracted with ethyl acetate. The combined organic layer was washed with brine, dried over anhydrous sodium sulfate and filtered. The filtrate was concentrated under reduced pressure. The residue was diluted in EtOH (250.0 mL). Then allyl bromide (13.8 g, 114.62 mmol) and Na₂CO₃ (9.1 g, 85.97 mmol) were added to the mixture. The mixture was stirred at room temperature for 48 h. The mixture was diluted with H₂O and extracted with EtOAc. The combined organic layer was washed with brine, dried over anhydrous sodium sulfate and filtered. The filtrate was evaporated in vacuo. The residue was purified by flash column chromatography with DCM/MeOH (10/1, v/v) to afford tert-butyl 4-{[(tert-butyldiphenylsilyl)oxy]methyl}-3-methylidene-2-oxopyrrolidine-1-carboxylate (5.0 g, 74%) as a colorless oil. LCMS (ESI, m/z): [M+H]⁺=466.2.

### Step 4. Synthesis of 4-{[(tert-butyldiphenylsilyl)oxy]methyl}-3-methylidenepyrrolidin-2-one

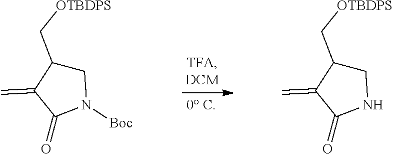

To a solution of tert-butyl 4-{[(tert-butyldiphenylsilyl)oxy]methyl}-3-methylidene-2-oxopyrrolidine-1-carboxylate (800.0 mg, 1.72 mmol) in DCM (10.0 mL) was added TFA (3.0 mL) at room temperature. The mixture was stirred at room temperature for 1 h. After the reaction was completed, the mixture was concentrated under reduced pressure to afford 4-{[(tert-butyldiphenylsilyl)oxy]methyl}-3-methylidenepyrrolidin-2-one (628.0 mg, crude) as a colorless oil. LCMS (ESI, m/z): [M+H]⁺=366.2.

### Step 5. Synthesis of 4-{[(tert-butyldiphenylsilyl)oxy]methyl}-1-{4-[(3-methyl-4-{[1,2,4]triazolo [1,5-a]pyridin-7-yloxy}phenyl)amino]quinazolin-6-yl}-3-methylidenepyrrolidin-2-one

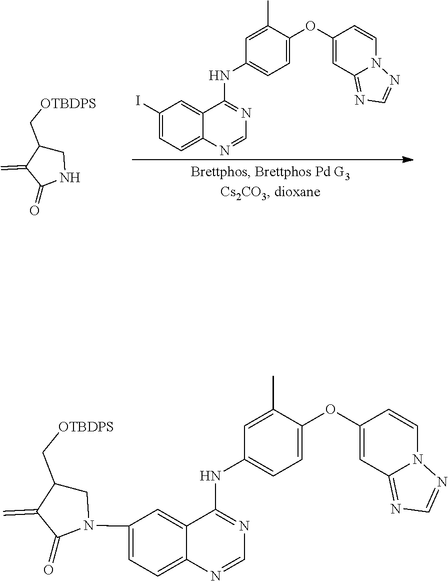

To a solution of 4-{[(tert-butyldiphenylsilyl)oxy]methyl}-3-methylidenepyrrolidin-2-one (628.0 mg, crude) in dioxane (14.0 mL) was added 6-iodo-N-(3-methyl-4-{[1,2,4]triazolo[1,5-a]pyridin-7-yloxy}phenyl)quinazolin-4-amine (424.6 mg, 0.86 mmol), Cs₂CO₃ (1119.5 mg, 3.44 mmol), BrettPhos (92.2 mg, 0.17 mmol) and BrettPhos Pd G3 (77.8 mg, 0.08 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford 4-{[(tert-butyldiphenylsilyl)oxy]methyl}-1-{4-[(3-methyl-4-{[1,2,4]triazolo[1,5-a]pyridin-7-yloxy}phenyl)amino]quinazolin-6-yl}-3-methylidenepyrrolidin-2-one (138.0 mg, 10%) as a light yellow solid. LCMS (ESI, m/z): [M+H]⁺=732.3.

### Step 6. Synthesis of 4-(hydroxymethyl)-1-{4-[(3-methyl-4-{[1,2,4]triazolo[1,5-a]pyridin-7-yloxy}phenyl)amino]quinazolin-6-yl}-3-methylidenepyrrolidin-2-one (Compound 29)

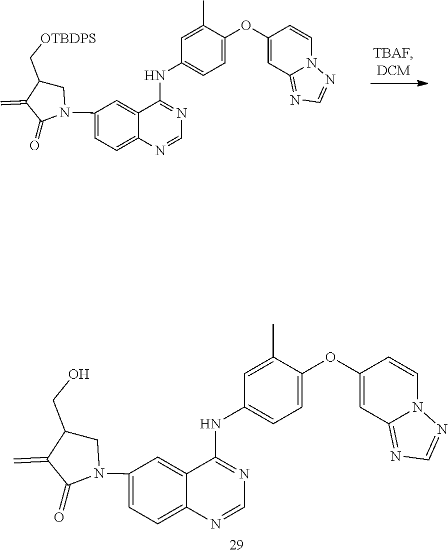

To a solution of 4-{[(tert-butyldiphenylsilyl)oxy]methyl}-1-{4-[(3-methyl-4-{[1,2,4]triazolo[1,5-a]pyridin-7-yloxy}phenyl)amino]quinazolin-6-yl}-3-methylidenepyrrolidin-2-one (100.0 mg, 0.14 mmol) in DCM (10.0 mL) was added TBAF (107.2 mg, 0.41 mmol) at room temperature. The resulting mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 22% B to 32% B in 8 min; Wave Length: 254 nm) to afford 4-(hydroxymethyl)-1-{4-[(3-methyl-4-{[1,2,4]triazolo[1,5-a]pyridin-7-yloxy}phenyl)amino]quinazolin-6-yl}-3-methylidenepyrrolidin-2-one (Compound 29) (5.3 mg, 7%) as a light yellow solid. LCMS (ESI, m/z): [M+H]⁺=494.2. ¹H NMR (400 MHz, DMSO-d₆): δ 9.92 (s, 1H), 8.94 (d, J=7.2 Hz, 1H), 8.80-8.77 (m, 1H), 8.59 (s, 1H), 8.39 (s, 1H), 8.34 (d, J=2.0 Hz, 1H), 7.86-7.81 (m, 3H), 7.25-7.23 (m, 1H), 7.06-7.03 (m, 1H), 6.82 (d, J=2.4 Hz, 1H), 6.05 (d, J=2.4 Hz, 1H), 5.63 (d, J=1.6 Hz, 1H), 5.10-5.07 (m, 1H), 4.19-4.14 (m, 1H), 3.95-3.91 (m, 1H), 3.74-3.70 (m, 1H), 3.61-3.55 (m, 1H), 3.28-3.26 (m, 1H), 2.22 (s, 3H).

### Example S29: Synthesis of 4 [(dimethylamino)methyl]-1-{4-[(3-methyl-4-{[1,2,4]triazolo[1,5-a]pyridin-7 yloxy}phenyl)amino]quinazolin-6-yl}-3-methylidenepyrrolidin-2-one; formic acid (Compound 30)

### Step 1. Synthesis of (1-{4-[(3-methyl-4-{[1,2,4]triazolo[1,5-a]pyridin-7-yloxy}phenyl)amino]quinazolin-6-yl}-4-methylidene-5-oxopyrrolidin-3-yl)methyl 4-methylbenzenesulfonate

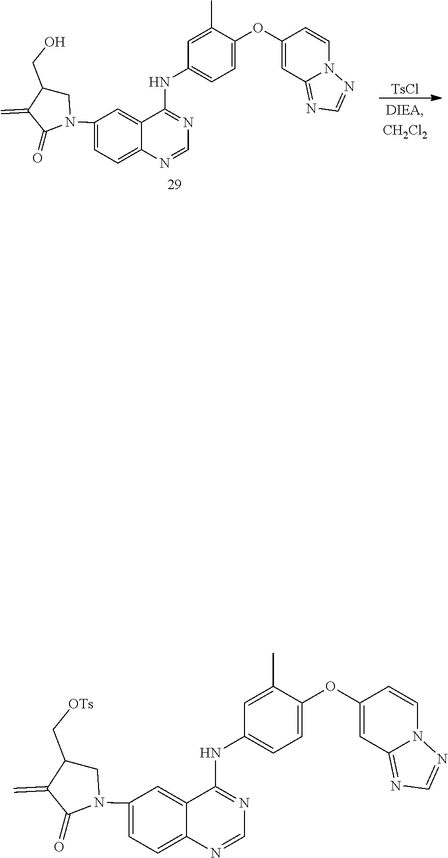

To a solution of 4-(hydroxymethyl)-1-{4-[(3-methyl-4-{[1,2,4]triazolo[1,5-a]pyridin-7-yloxy}phenyl)amino]quinazolin-6-yl}-3-methylidenepyrrolidin-2-one (300.0 mg, 0.61 mmol) in CH₂Cl₂ (20.0 mL) was added DIEA (785.6 mg, 6.08 mmol) and p-toluenesulfonyl chloride (1158.8 mg, 6.08 mmol) at 0° C. The resulting mixture was stirred at room temperature for 16 h. After the reaction was completed, the mixture was concentrated under vacuum. The residue was purified by flash column chromatography with CH₂Cl₂/MeOH (10/1, v/v) to afford (1-{4-[(3-methyl-4-{[1,2,4]triazolo[1,5-a]pyridin-7-yloxy}phenyl)amino]quinazolin-6-yl}-4-methylidene-5-oxopyrrolidin-3-yl)methyl 4-methylbenzenesulfonate (150.0 mg, 38%) as a light yellow solid. LCMS (ESI, m/z): [M+H]⁺=648.2.

### Step 2. Synthesis of 4-[(dimethylamino)methyl]-1-{4-[(3-methyl-4-{[1,2,4]triazolo [1,5-a]pyridin-7-yloxy}phenyl)amino]quinazolin-6-yl}-3-methylidenepyrrolidin-2-one; formic acid (Compound 30)

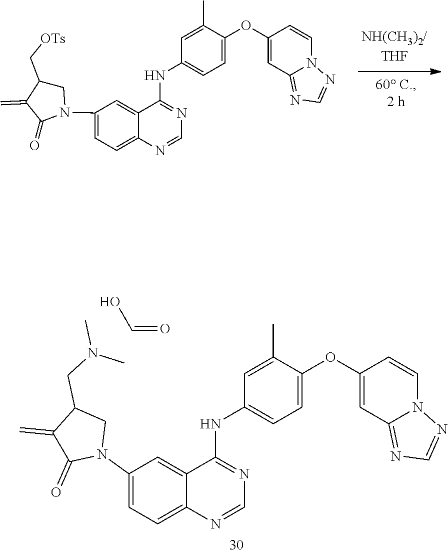

To a solution of (1-{4-[(3-methyl-4-{[1,2,4]triazolo[1,5-a]pyridin-7-yloxy}phenyl)amino]quinazolin-6-yl}-4-methylidene-5-oxopyrrolidin-3-yl)methyl 4-methylbenzenesulfonate (130.0 mg, 0.20 mmol) was added in dimethylamine (5.0 mL, 2 M in THF) at room temperature. The resulting mixture was stirred at 60° C. for 2 h. After the reaction was completed, the resulting mixture was concentrated under reduced pressure. The residue was purified by Prep-HPLC with the following conditions (Column: Xselect CSH OBD Column 30×150 mm, 5 um; Mobile Phase A: Water (0.1% FA), Mobile Phase B: MeOH-HPLC; Flow rate: 60 mL/min; Gradient: 10% B to 30% B in 12 min; Wave Length: 254 nm) to afford 4-[(dimethylamino)methyl]-1-{4-[(3-methyl-4-{[1,2,4]triazolo[1,5-a]pyridin-7-yloxy}phenyl)amino]quinazolin-6-yl}-3-methylidenepyrrolidin-2-one; formic acid (Compound 30) (2.6 mg, 2%) as a yellow solid. LCMS (ESI, m/z): [M+H]⁺=521.2. ¹H NMR (400 MHz, DMSO-d₆): δ 9.99 (s, 1H), 8.94 (d, J=7.6 Hz, 1H), 8.81-8.78 (m, 1H), 8.57 (s, 1H), 8.38 (s, 1H), 8.32 (d, J=1.6 Hz, 1H), 8.14 (s, 1H), 7.84-7.79 (m, 3H), 7.24-7.22 (m, 1H), 7.05-7.03 (m, 1H), 6.81 (d, J=2.4 Hz, 1H), 6.03 (d, J=2.4 Hz, 1H), 5.60 (d, J=1.6 Hz, 1H), 4.19-4.14 (m, 1H), 3.91-3.87 (m, 1H), 2.67-2.55 (m, 1H), 2.31 (s, 6H), 2.21 (s, 3H).

### Example S30: Synthesis of (4R)-4-methyl-1-{4-[(3-methyl-4-{[1,2,4]triazolo[1,5-a]pyridin-7-yloxyphenyl)amino]pyrido[3,4-d]pyrimidin-6 yl}-3-methylidenepyrrolidin-2-one (Compound 31)

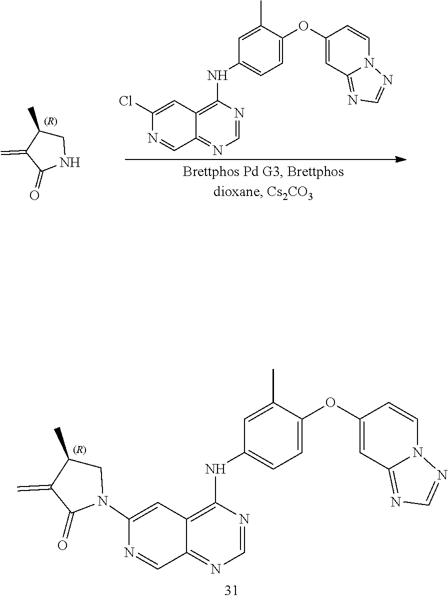

A mixture of 6-chloro-N-(3-methyl-4-{[1,2,4]triazolo[1,5-a]pyridin-7-yloxy}phenyl)pyrido[3,4-d]pyrimidin-4-amine (80.0 mg, 0.20 mmol), (4R)-4-methyl-3-methylidenepyrrolidin-2-one (26.4 mg, crude), Cs₂CO₃ (193.6 mg, 0.59 mmol), BrettPhos (42.5 mg, 0.08 mmol) and BrettPhos Pd G3 (35.9 mg, 0.04 mmol) in 1,4-dioxane (4.0 mL) was stirred at 100° C. for 2 h under N₂. After the reaction was completed, the resulting mixture was concentrated under reduced pressure. The residue was purified by flash column chromatography with CH₂Cl₂/MeOH (10/1, v/v) and then purified by Prep-HPLC with the following conditions (Column: XSelect CSH Prep C18 OBD Column, 19×250 mm, 5 μm; Mobile Phase A: Water (10 mmol/L NH₄HCO₃), Mobile Phase B: MeOH-HPLC; Flow rate: 25 mL/min; Gradient: 75% B to 80% B in 10 min; Wave Length: 254 nm) to afford (4R)-4-methyl-1-{4-[(3-methyl-4 {[1,2,4]triazolo[1,5-a]pyridin-7-yloxy}phenyl)amino]pyrido[3,4-d]pyrimidin-6-yl}-3-methylidenepyrrolidin-2-one (Compound 31) (6.3 mg, 6%) as a white solid. LCMS (ESI, m/z): [M+H]⁺=479.3. ¹H NMR (400 MHz, DMSO-d₆): δ 10.34 (s, 1H), 9.20 (s, 1H), 9.09 (s, 1H), 8.95 (d, J=7.6 Hz, 1H), 8.66 (s, 1H), 8.39 (s, 1H), 7.87-7.82 (m, 2H), 7.24 (d, J=8.8 Hz, 1H), 7.06-7.03 (m, 1H), 6.83 (d, J=2.4 Hz, 1H), 6.07 (d, J=2.8 Hz, 1H), 5.60 (d, J=2.4 Hz, 1H), 4.40-4.35 (m, 1H), 3.70-3.66 (m, 1H), 3.17-3.13 (m, 1H), 2.22 (s, 3H), 1.33 (d, J=6.8 Hz, 3H).

### Example S31: Synthesis of (4S)-4-methyl-1-{4-[(3-methyl-4-{[1,2,4]triazolo[1,5-a]pyridin-7-yloxyphenyl)amino]pyrido[3,4-d]pyrimidin-6 yl}-3-methylidenepyrrolidin-2-one (Compound 32)

### Step 1. Synthesis of (4S)-4-methyl-1-{4-[(3-methyl-4-{[1,2,4]triazolo[1,5-a]pyridin-7-yloxy}phenyl)amino]pyrido[3,4-d]pyrimidin-6-yl}-3-methylidenepyrrolidin-2-one (Compound 32)

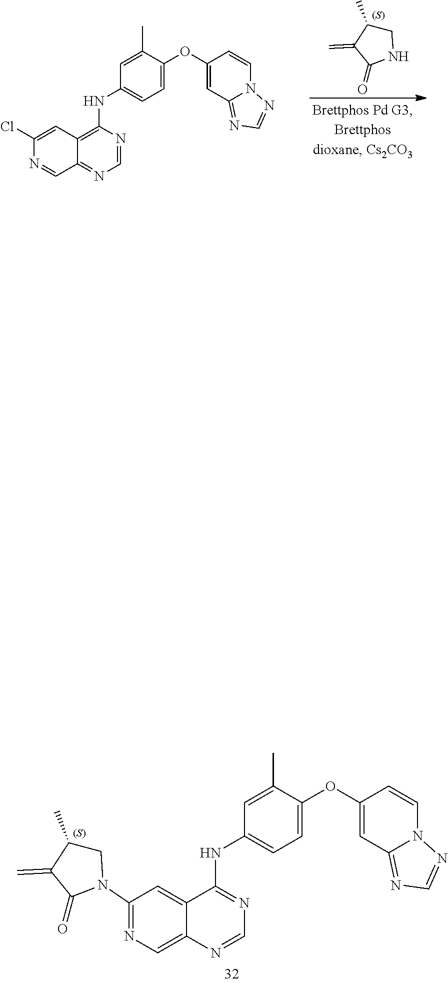

A mixture of 6-chloro-N-(3-methyl-4-{[1,2,4]triazolo[1,5-a]pyridin-7-yloxy}phenyl)pyrido[3,4-d]pyrimidin-4-amine (300.0 mg, 0.74 mmol), (4S)-4-methyl-3-methylidenepyrrolidin-2-one (180.0 mg, crude), Cs₂CO₃ (726.14 mg, 2.22 mmol), BrettPhos (159.4 mg, 0.30 mmol) and BrettPhos Pd G3 (134.6 mg, 0.16 mmol) in dioxane (15.0 mL) was stirred at 100° C. for 2 h under N₂. After the reaction was completed, the resulting mixture was concentrated under reduced pressure. The residue was purified by flash column chromatography with CH₂Cl₂/CH₃OH (85/15, v/v) and then purified by Prep-CHIRAL-HPLC with the following conditions (Column: CHIRAL ART Amylose-SA, 2×25 cm, 5 μm; Mobile Phase A: Hex:MtBE=1:1 (0.5% 2M NH₃-MEOH), Mobile Phase B: EtOH-HPLC; Flow rate: 20 mL/min; Gradient: 20% B to 20% B in 25 min; Wave Length: 220/254 nm; RT(min): 23.67) to afford (4S)-4-methyl-1-{4-[(3-methyl-4-{[1,2,4]triazolo[1,5-a]pyridin-7-yloxy}phenyl)amino]pyrido[3,4-d]pyrimidin-6-yl}-3-methylidenepyrrolidin-2-one (Compound 32) (19.3 mg, 5%) as a white solid. LCMS (ESI, m/z): [M+H]⁺=479.3. ¹H NMR (400 MHz, DMSO-d₆): δ 10.35-10.32 (m, 1H), 9.20-8.93 (m, 3H), 8.65-8.63 (m, 1H), 8.39 (s, 1H), 7.86-7.82 (m, 2H), 7.24-7.21 (m, 1H), 7.05-7.03 (m, 1H), 6.84 (s, 1H), 6.06 (s, 1H), 5.59 (s, 1H), 4.39-4.33 (m, 1H), 3.70-3.66 (m, 1H), 3.17-3.10 (m, 1H), 2.21 (s, 3H), 1.33-1.31 (m, 3H).

### Example S32: Synthesis of (E)-3-(2-(dimethylamino)ethylidene)-1-(4-((3-methyl-4-((1-methyl-1H-benzo[d]imidazol-5 yl)oxy)phenyl)amino)quinazolin-6 yl)pyrrolidin-2-one (Compound 33)

### Step 1. Synthesis of (E)-3-(2-(dimethylamino)ethylidene)-1-(4-((3-methyl-4-((1-methyl-1H-benzo[d]imidazol-5-yl)oxy)phenyl)amino)quinazolin-6-yl)pyrrolidin-2-one (Compound 33)

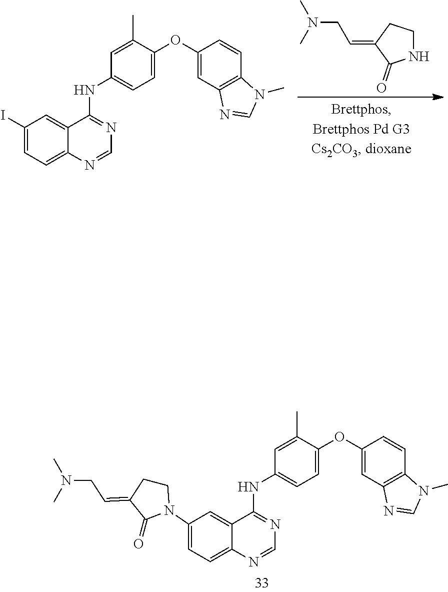

To a solution of 6-iodo-N-(3-methyl-4-((1-methyl-1H-benzo[d]imidazol-5-yl)oxy)phenyl)quinazolin-4-amine (200.0 mg, 0.39 mmol) in 1,4-dioxane (10.0 mL) was added (E)-3-(2-(dimethylamino)ethylidene)pyrrolidin-2-one (182.4 mg, 1.18 mmol), Cs₂CO₃ (385.3 mg, 1.18 mmol), Brettphos (42.3 mg, 0.08 mmol) and Brettphos Pd G3 (35.7 mg, 0.04 mmol) at room temperature under N₂. The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (80/20, v/v) and then purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 25% B to 34% B in 8 min; Wave Length: 254 nm) to afford (E)-3-(2-(dimethylamino)ethylidene)-1-(4-((3-methyl-4-((1-methyl-1H-benzo[d]imidazol-5-yl)oxy)phenyl)amino)quinazolin-6-yl)pyrrolidin-2-one (Compound 33) (10.5 mg, 5%) as a white solid. LCMS (ESI, m/z): [M+H]⁺=534.3. ¹H NMR (400 MHz, DMSO-d₆): δ 9.78 (s, 1H), 8.73-8.71 (m, 1H), 8.53 (s, 1H), 8.31 (d, J=2.0 Hz, 1H), 8.18 (s, 1H), 7.81 (d, J=9.2 Hz, 1H), 7.70 (d, J=2.4 Hz, 1H), 7.62-7.57 (m, 2H), 7.12 (d, J=2.0 Hz, 1H), 7.03-7.00 (m, 1H), 6.89 (d, J=8.8 Hz, 1H), 6.49-6.46 (m, 1H), 4.09-4.05 (m, 2H), 3.85 (s, 3H), 3.07-3.00 (m, 2H), 2.98-2.80 (m, 2H), 2.27 (s, 3H), 2.20 (s, 6H).

### Example S33: Synthesis of (1R,5R)-2-[4-({3-methyl-4 [(1-methyl-1,3-benzodiazol-5-yl)oxy]pyridinenyl}amino)quinazolin-6-yl]-4-methylidene-2-azabicyclo[3.1.0]hexan-3-one and (1S,5S)-2-[4-([3-methyl-4[(1-methyl-1,3-benzodiazol-5-yl)oxy]pyridinenyl}amino)quinazolin-6-yl]-4-methylidene-2-azabicyclo[3.1.0]hexan-3-one (Compounds 34 and 35)

### Step 1. Synthesis of 2-[4-({3-methyl-4-[(1-methyl-1,3-benzodiazol-5-yl)oxy]phenyl}amino)quinazolin-6-yl]-4-methylidene-2-azabicyclo[3.1.0]hexan-3-one

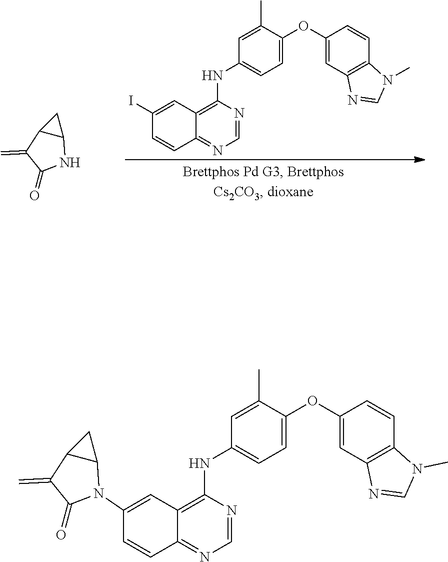

To a solution of 4-methylidene-2-azabicyclo[3.1.0]hexan-3-one (258.1 mg, 2.36 mmol) in dioxane (30.0 mL) was added 6-iodo-N-(3-methyl-4-((1-methyl-1H-benzo[d]imidazol-5-yl)oxy)phenyl)quinazolin-4-amine (400.0 mg, 0.79 mmol), Cs₂CO₃ (770.7 mg, 2.36 mmol), BrettPhos (152.2 mg, 0.29 mmol) and Brettphos Pd G3 (128.3 mg, 0.14 mmol) at room temperature under N₂. The resulting mixture was stirred at 100° C. for 2 h under N₂. After the reaction was completed, the resulting mixture was filtered. The filtrate was concentrated under reduced pressure. The residue was purified by flash chromatography with CH₂Cl₂/MeOH (90/10, v/v) to afford 2-[4-({3-methyl-4-[(1-methyl-1,3-benzodiazol-5-yl)oxy]phenyl}amino)quinazolin-6-yl]-4-methylidene-2-azabicyclo[3.1.0]hexan-3-one (40.0 mg, 10%) as a white solid. LCMS (ESI, m/z): [M+H]⁺=489.2.

### Step 2: Synthesis of (1R,5R)-2-[4-({3-methyl-4-[(1-methyl-1,3-benzodiazol-5-yl)oxy]phenyl}amino)quinazolin-6-yl]-4-methylidene-2-azabicyclo[3.1.0]hexan-3-one and (1S,5S)-2-[4-({3-methyl-4-[(1-methyl-1,3-benzodiazol-5-yl)oxy]phenyl}amino)quinazolin-6-yl]-4-methylidene-2-azabicyclo[3.1.0]hexan-3-one (Compounds 34 and 35)

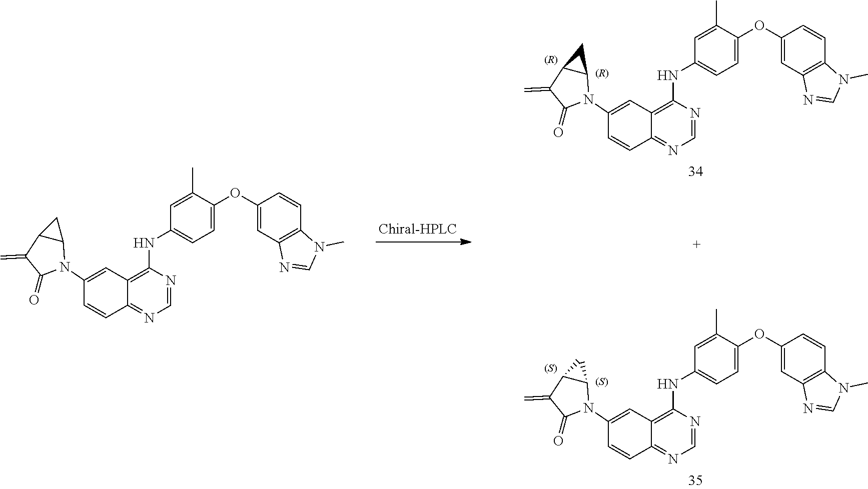

The racemic product 2-[4-({3-methyl-4-[(1-methyl-1,3-benzodiazol-5-yl)oxy]phenyl}amino)quinazolin-6-yl]-4-methylidene-2-azabicyclo[3.1.0]hexan-3-one (100.0 mg, 0.21 mmol) was separated by Prep-Chiral-HPLC with the following conditions (Column: CHIRALPAK IG, 2×25 cm, 5 μm; Mobile Phase A: Hex (0.5% 2M NH₃-MeOH)-HPLC, Mobile Phase B: EtOH: DCM=1: 1-HPLC; Flow rate: 20 mL/min; Gradient: 55% B to 55% B in 16 min; Wave Length: 220/254 nm; RT1(min): 10.36; RT2(min): 13.06) to afford 2-[4-({3-methyl-4-[(1-methyl-1,3-benzodiazol-5-yl)oxy]phenyl}amino)quinazolin-6-yl]-4-methylidene-2-azabicyclo[3.1.0]hexan-3-one Enantiomer 1 (Retention time: 10.36 min, 18.3 mg, 36%) as a white solid and 2-[4-({3-methyl-4-[(1-methyl-1,3-benzodiazol-5-yl)oxy]phenyl}amino)quinazolin-6-yl]-4-methylidene-2-azabicyclo[3.1.0]hexan-3-one Enantiomer 2 (Retention time: 13.06 min, 18.8 mg, 37%) as a white solid. The absolute stereochemistry of Enantiomers 1 and 2 was not assigned. The two enantiomeric structures that could be obtained from chiral separation of the enantiomeric mixture as described above are shown as Compounds 34 and 35 in Table 1.

2-[4-({3-methyl-4-[(1-methyl-1,3-benzodiazol-5-yl)oxy]phenyl}amino)quinazolin-6-yl]-4-methylidene-2-azabicyclo[3.1.0]hexan-3-one Enantiomer 1: RT1(min): 10.36; LCMS (ESI, m/z): [M+H]⁺=489.2. ¹H NMR (400 MHz, DMSO-d₆): δ 10.00 (s, 1H), 8.59-8.57 (m, 2H), 8.49-8.46 (m, 1H), 8.26 (s, 1H), 7.85 (d, J=8.8 Hz, 1H), 7.69 (s, 1H), 7.62-7.57 (m, 2H), 7.13 (d, J=2.0 Hz, 1H), 7.06-7.03 (m, 1H), 6.90 (d, J=8.4 Hz, 1H), 6.01 (s, 1H), 5.75 (s, 1H), 4.07-4.04 (m, 1H), 3.86 (s, 3H), 2.58-2.52 (m, 1H), 2.27 (s, 3H), 1.47-1.45 (m, 1H), 0.99-0.98 (m, 1H).

2-[4-({3-methyl-4-[(1-methyl-1,3-benzodiazol-5-yl)oxy]phenyl}amino)quinazolin-6-yl]-4-methylidene-2-azabicyclo[3.1.0]hexan-3-one Enantiomer 2: RT2(min): 13.06; LCMS (ESI, m/z): [M+H]⁺=489.2. ¹H NMR (400 MHz, DMSO-d₆): δ 9.86 (s, 1H), 8.57-8.53 (m, 2H), 8.46-8.44 (m, 1H), 8.18 (s, 1H), 7.84 (d, J=9.2 Hz, 1H), 7.69 (s, 1H), 7.60-7.57 (m, 2H), 7.12 (d, J=2.4 Hz, 1H), 7.03-7.00 (m, 1H), 6.88 (d, J=8.8 Hz, 1H), 6.00 (s, 1H), 5.74 (s, 1H), 4.07-4.04 (m, 1H), 3.85 (s, 3H), 2.58-2.52 (m, 1H), 2.27 (s, 3H), 1.47-1.44 (m, 1H), 0.99-0.98 (m, 1H).

### Example S34: Synthesis of (S)-1-(4-((4-([1,2,4]triazolo[1,5-a]pyridin-7 ylmethyl)-3-methylphenyl)amino)quinazolin-6-yl)-4-methyl-3-methylenepyrrolidin-2-one (Compound 36)

### Step 1. Synthesis of N-(4-([1,2,4]triazolo[1,5-a]pyridin-7-ylmethyl)-3-methylphenyl)-6-iodoquinazolin-4-amine

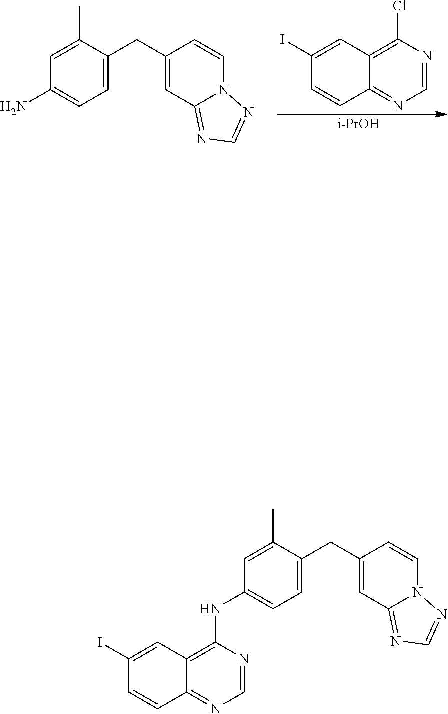 [SMILES: "i-PrOH" ; Cc1cc(N)ccc1Cc1ccn2ncnc2c1 ; Clc1ncnc2ccc(I)cc12 ; Cc1cc(Nc2ncnc3ccc(I)cc23)ccc1Cc1ccn2ncnc2c1]

To a solution of 3-methyl-4-{[1,2,4]triazolo[1,5-a]pyridin-7-ylmethyl}aniline (300.0 mg, 1.25 mmol) in isopropyl alcohol (10.0 mL) was added 4-chloro-6-iodoquinazoline (365.7 mg, 1.25 mmol) at room temperature. The resulting mixture was stirred at room temperature for 1 h. After the reaction was completed, the mixture was concentrated under reduced pressure. The residue was purified by flash column chromatography with CH₂Cl₂/MeOH (80/20, v/v) to afford N-(4-([1,2,4]triazolo[1,5-a]pyridin-7-ylmethyl)-3-methylphenyl)-6-iodoquinazolin-4-amine (323.0 mg, 65%) as a yellow solid. LCMS (ESI, m/z): [M+H]⁺=493.1.

### Step 2. Synthesis of (S)-1-(4-((4-([1,2,4]triazolo[1,5-a]pyridin-7-ylmethyl)-3-methylphenyl)amino)quinazolin-6-yl)-4-methyl-3-methylenepyrrolidin-2-one (Compound 36)

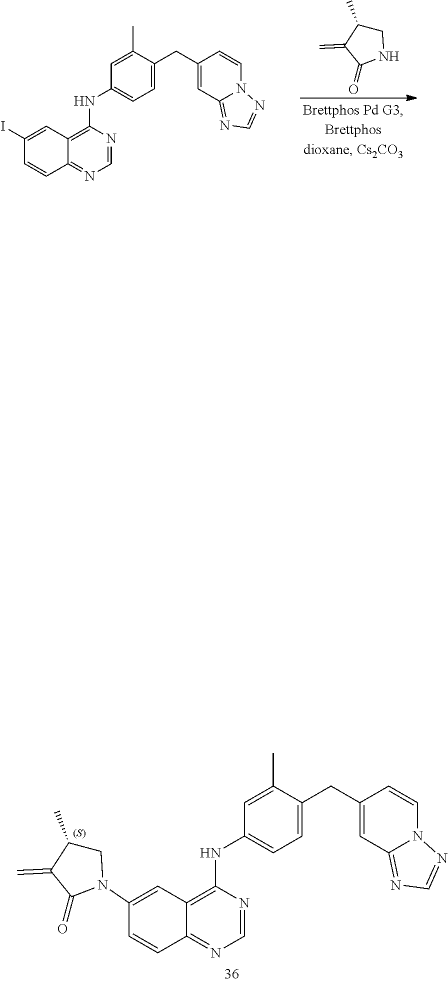

To a solution of 6-iodo-N-(3-methyl-4-{[1,2,4]triazolo[1,5-a]pyridin-7-ylmethyl}phenyl)quinazolin-4-amine (100.0 mg, 0.20 mmol) in 1,4-dioxane (10.0 mL) was added (4S)-4-methyl-3-methylidenepyrrolidin-2-one (45.1 mg, 0.40 mmol), BrettPhos (43.6 mg, 0.08 mmol), Brettphos Pd G3 (29.7 mg, 0.04 mmol) and Cs₂CO₃ (187.8 mg, 0.60 mmol) at room temperature under N₂. The mixture was stirred at 100° C. for 16 h. After the reaction was completed, the resulting mixture was concentrated under reduced pressure. The residue was purified by phase flash column chromatography with CH₂Cl₂/MeOH (80/20, v/v) and then purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 32% B to 32% B in 8 min; Wave Length: 254 nm) to afford (4S)-4-methyl-1-{4-[(3-methyl-4-{[1,2,4]triazolo[1,5-a]pyridin-7-ylmethyl}phenyl)amino]quinazolin-6-yl}-3-methylidenepyrrolidin-2-one (Compound 36) (5.5 mg, 5%) as a white solid. LCMS (ESI, m/z): [M+H]⁺=476.2. ¹H NMR (400 MHz, DMSO-d₆): δ 9.77 (s, 1H), 8.87 (d, J=6.8 Hz, 1H), 8.71-8.68 (m, 1H), 8.54 (s, 1H), 8.44 (s, 1H), 8.29 (d, J=1.6 Hz, 1H), 7.82 (d, J=9.2 Hz, 1H), 7.68-7.66 (m, 1H), 7.61 (s, 1H), 7.53 (s, 1H), 7.28 (d, J=8.4 Hz, 1H), 7.04-7.02 (m, 1H), 5.99 (d, J=2.8 Hz, 1H), 5.55 (d, J=2.0 Hz, 1H), 4.23-4.15 (m, 3H), 3.66-3.62 (m, 1H), 3.20-3.17 (m, 1H), 2.22 (s, 3H), 1.34 (d, J=6.0 Hz, 3H).

### Example S35: Synthesis of (S)-4-methyl-1-(4-((3-methyl-4-((1-methyl-1H-benzo[d]imidazol-5-yl)oxy)phenyl)amino)quinazolin-6 yl)-3-methylenepyrrolidin-2-one (Compound 37)

### Step 1. Synthesis of 6-iodo-N-(3-methyl-4-((1-methyl-1H-benzo[d]imidazol-5-yl)oxy)phenyl)quinazolin-4-amine

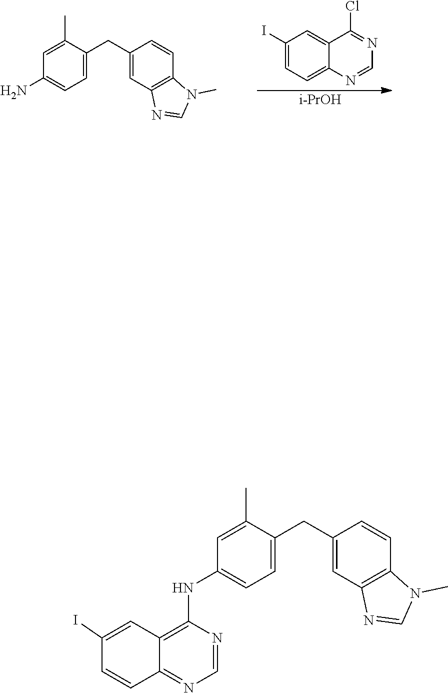 [SMILES: "i-PrOH" ; Cc1cc(N)ccc1Cc1ccc2c(c1)ncn2C ; Clc1ncnc2ccc(I)cc12 ; Cc1cc(Nc2ncnc3ccc(I)cc23)ccc1Cc1ccc2c(c1)ncn2C]

To a solution of 3-methyl-4-[(1-methyl-1,3-benzodiazol-5-yl)oxy]aniline (1.0 g, 3.94 mmol) in isopropyl alcohol (15.0 mL) was added 4-chloro-6-iodoquinazoline (1.1 g, 3.94 mmol) at room temperature. The resulting mixture was stirred at room temperature for 1 h. After the reaction was completed, the mixture was concentrated under reduced pressure. The residue was purified by flash column chromatography with CH₂Cl₂/MeOH (80/20, v/v) to afford 6-iodo-N-{3-methyl-4-[(1-methyl-1,3-benzodiazol-5-yl)oxy]phenyl}quinazolin-4-amine (1.1 g, 54%) as a yellow solid. LCMS (ESI, m/z): [M+H]⁺=508.1.

### Step 2: Synthesis of (S)-4-methyl-1-(4-((3-methyl-4-((1-methyl-1H-benzo [d]imidazol-5-yl)oxy)phenyl)amino)quinazolin-6-yl)-3-methylenepyrrolidin-2-one (Compound 37)

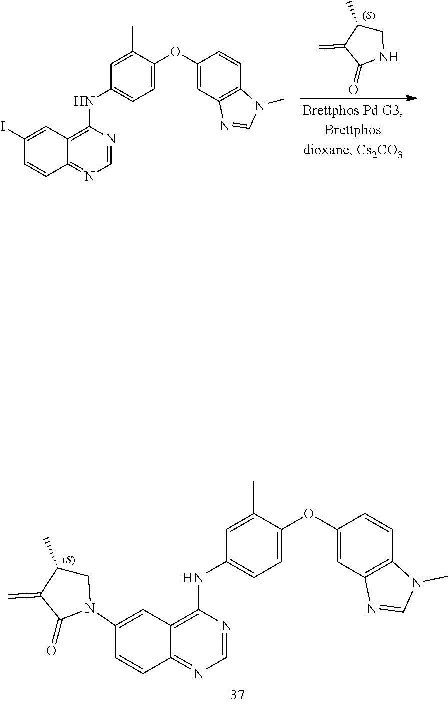

To a solution of 6-iodo-N-{3-methyl-4-[(1-methyl-1,3-benzodiazol-5-yl)oxy]phenyl}quinazolin-4-amine (200.0 mg, 0.39 mmol) in 1,4-dioxane (10.0 mL) was added (4S)-4-methyl-3-methylidenepyrrolidin-2-one (65.7 mg, 0.59 mmol), BrettPhos Pd G3 (71.4 mg, 0.08 mmol), BrettPhos (84.6 mg, 0.16 mmol) and Cs₂CO₃ (256.8 mg, 0.78 mmol) at room temperature under N₂. The resulting mixture was stirred at 100° C. for 16 h under N₂. After the reaction was completed, the mixture was concentrated under reduced pressure. The residue was purified by flash chromatography with CH₂Cl₂/MeOH (90/10, v/v) and then purified by Prep-HPLC with the following conditions: (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 35% B to 55% B in 8 min; 254 nm) to afford (4S)-4-methyl-1-[4-({3-methyl-4-[(1-methyl-1,3-benzodiazol-5-yl)oxy]phenyl}amino)quinazolin-6-yl]-3-methylidenepyrrolidin-2-one (Compound 37) (38.0 mg, 19%) as a white solid. LCMS (ESI, m/z): [M+H]⁺=491.3. ¹H NMR (400 MHz, DMSO-d₆): δ 9.78 (s, 1H), 8.71-8.69 (m, 1H), 8.54 (s, 1H), 8.29 (d, J=2.0 Hz, 1H), 8.18 (s, 1H), 7.82 (d, J=9.2 Hz, 1H), 7.69 (d, J=2.0 Hz, 1H), 7.62-7.57 (m, 2H), 7.12 (d, J=2.0 Hz, 1H), 7.03-7.01 (m, 1H), 6.89 (d, J=8.8 Hz, 1H), 6.00 (d, J=2.8 Hz, 1H), 5.56 (d, J=2.0 Hz, 1H), 4.23-4.19 (m, 1H), 3.85 (s, 3H), 3.66-3.62 (m, 1H), 3.23-3.19 (m, 1H), 2.27 (s, 3H), 1.36-1.32 (m, 3H).

### Example S36: Synthesis of (3E)-3-[2-(dimethylamino)ethylidene]-1-[4-({3-methyl-4-[(1-methyl-1,3-benzodiazol-5-yl)oxy]pyridinenyl}amino)pyrido[3,2-d]pyrimidin-6-yl]pyrrolidin-2-one (Compound 38)

### Step 1. Synthesis of (3E)-3-[2-(dimethylamino)ethylidene]-1-[4-({3-methyl-4-[(1-methyl-1,3-benzodiazol-5-yl)oxy]phenyl}amino)pyrido[3,2-d]pyrimidin-6-yl]pyrrolidin-2-one (Compound 38)

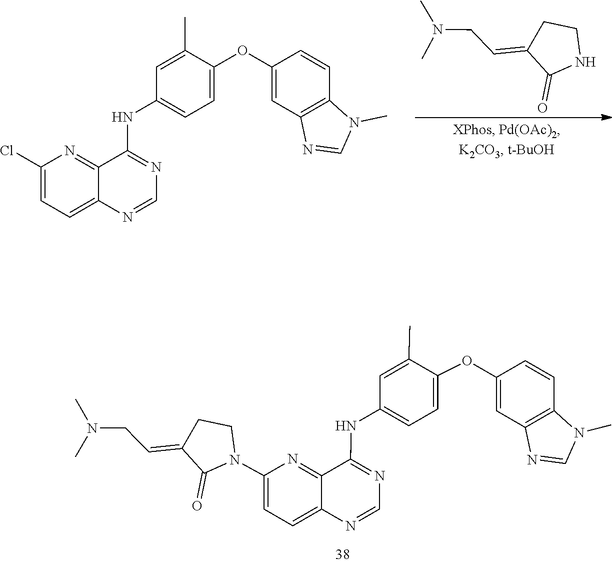

To a solution of 6-chloro-N-{3-methyl-4-[(1-methyl-1,3-benzodiazol-5-yl)oxy]phenyl}pyrido[3,2-d]pyrimidin-4-amine (150.0 mg, 0.36 mmol) in t-BuOH (10.0 mL) was added (3E)-3-[2-(dimethylamino)ethylidene]pyrrolidin-2-one (166.5 mg, 1.08 mmol), K₂CO₃ (149.2 mg, 1.08 mmol), XPhos (34.3 mg, 0.07 mmol) and Pd(OAc)₂ (8.1 mg, 0.04 mmol) at room temperature under N₂. The resulting mixture was stirred at 100° C. for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions Column: (Xselect CSH OBD Column 30×150 mm, 5 um; Mobile Phase A: Water (10 mmol/L NH₄HCO₃), Mobile Phase B: ACN; Flow rate:60 mL/min; Gradient:32% B to 42% B in 8 min; 254 nm) to afford (3E)-3-[2-(dimethylamino)ethylidene]-1-[4-({3-methyl-4-[(1-methyl-1,3-benzodiazol-5-yl)oxy]phenyl}amino)pyrido[3,2-d]pyrimidin-6-yl]pyrrolidin-2-one (Compound 38) (26.3 mg, 14%) as a white solid. LCMS (ESI, m/z): [M+H]⁺=535.3. ¹H NMR (400 MHz, DMSO-d₆): δ 9.51 (s, 1H), 8.97 (d, J=9.6 Hz, 1H), 8.59 (s, 1H), 8.24 (d, J=9.6 Hz, 1H), 8.17 (s, 1H), 7.83 (d, J=2.4 Hz, 1H), 7.79-7.76 (m, 1H), 7.57 (d, J=8.4 Hz, 1H), 7.11 (d, J=2.0 Hz, 1H), 7.01 (d, J=2.0 Hz, 1H), 7.00-6.90 (m, 1H), 6.58-6.55 (m, 1H), 4.35-4.32 (m, 2H), 3.84 (s, 3H), 3.12 (d, J=6.4 Hz, 2H), 2.94-2.90 (m, 2H), 2.27 (s, 3H), 2.21 (s, 6H).

### Example S37: Synthesis of 1-(4-((3-methyl-4-((1-methyl-1H-benzo[d]imidazol-5-yl)oxy)phenyl)amino)pyrido[3,4-d]pyrimidin-6 yl)-3-methylenepyrrolidin-2-one (Compound 39)

### Step 1. Synthesis of 1-(4-((3-methyl-4-((1-methyl-1H-benzo[d]imidazol-5-yl)oxy)phenyl)amino)pyrido[3,4-d]pyrimidin-6-yl)-3-methylenepyrrolidin-2-one (Compound 39)

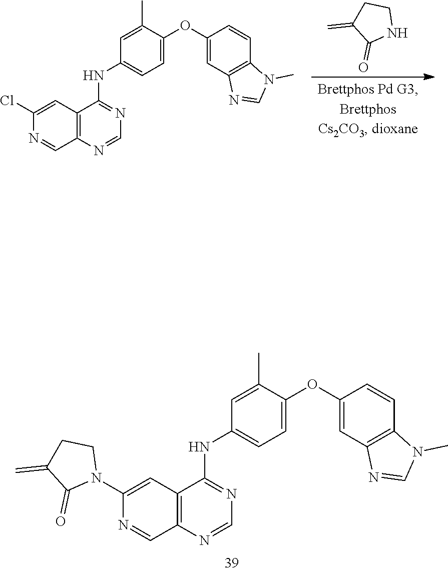

To a solution of 6-chloro-N-{3-methyl-4-[(1-methyl-1,3-benzodiazol-5-yl)oxy]phenyl}pyrido[3,4-d]pyrimidin-4-amine (80.0 mg, 0.19 mmol) in 1,4-dioxane (8.0 mL) was added 3-methylidenepyrrolidin-2-one (27.9 mg, 0.28 mmol), Cs₂CO₃ (125.0 mg, 0.38 mmol), BrettPhos (20.6 mg, 0.03 mmol) and BrettPhos Pd G3 (69.5 mg, 0.07 mmol) at room temperature. The resulting mixture was stirred at 100° C. for 16 h under N₂. After the reaction was completed, the resulting mixture was concentrated under reduced pressure. The residue was purified by flash column chromatography with CH₂Cl₂/CH₃OH (90/10, v/v) and then purified by Prep-HPLC with the following conditions: (Column: XBridge Prep Phenyl OBD Column, 19×250 mm, 5 μm; Mobile Phase A: Water (10 mmol/L NH₄HCO₃), Mobile Phase B: ACN; Flow rate: 25 mL/min; Gradient: 43% B to 43% B in 10 min; Wave Length: 254 nm) to afford 1-[4-({3-methyl-4-[(1-methyl-1,3-benzodiazol-5-yl)oxy]phenyl}amino)pyrido[3,4-d]pyrimidin-6-yl]-3-methylidenepyrrolidin-2-one (Compound 39) (9.2 mg, 10%) as a yellow solid. LCMS (ESI, m/z): [M+H]⁺=478.2. ¹H NMR (400 MHz, DMSO-d₆): δ 10.29 (s, 1H), 9.19 (s, 1H), 9.07 (s, 1H), 8.60 (s, 1H), 8.18 (s, 1H), 7.73 (d, J=2.0 Hz, 1H), 7.62-7.57 (m, 2H), 7.13 (d, J=2.0 Hz, 1H), 7.03-7.00 (m, 1H), 6.86 (d, J=8.8 Hz, 1H), 6.04 (s, 1H), 5.59 (s, 1H), 4.17-4.13 (m, 2H), 3.84 (s, 3H), 2.96-2.92 (m, 2H), 2.26 (s, 3H).

### Example S38: Synthesis of (3E)-1-{4-[(3-methyl-4-{[1,2,4]triazolo[1,5-a]pyridin-7-yloxy}phenyl)amino]quinazolin-6-yl}-3 [3-(4-methylpiperazin-1 yl)propylidene]pyrrolidin-2-one (Compound 40)

### Step 1. Synthesis of tert-butyl 3-{3-[(tert-butyldimethylsilyl)oxy]-1-hydroxypropyl}-2-oxopyrrolidine-1-carboxylate

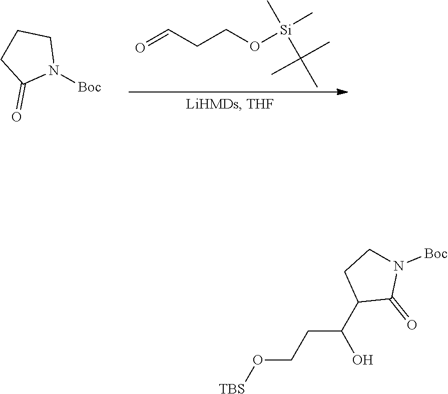

To a solution of tert-butyl 2-oxopyrrolidine-1-carboxylate (20.0 g, 107.98 mmol) in THF (200.0 mL) was added LiHMDS (129.4 mL, 1.0 mol/L) at −78° C. under N₂. The resulting mixture was stirred at −78° C. for 1 h. Then a solution of 3-[(tert-butyldimethylsilyl)oxy]propanal (20.3 g, 107.98 mmol) in THF (50.0 mL) was added dropwise to the mixture at −78° C. The mixture was stirred at −78° C. for additional 1 h. After the reaction was completed, the reaction mixture was quenched with NH₄Cl (sat.) solution and extracted with ethyl acetate. The combined organic layer was washed with brine, dried over anhydrous sodium sulfate and filtered. The filtrate was evaporated in vacuo. The residue was purified by flash column chromatography with petroleum ether/ethyl acetate (3/7, v/v) to afford tert-butyl 3-{3-[(tert-butyldimethylsilyl)oxy]-1-hydroxypropyl}-2-oxopyrrolidine-1-carboxylate (12.8 g, 31%) as a colorless oil. LCMS (ESI, m/z): [M+H]⁺=374.2.

### Step 2. Synthesis of tert-butyl 3-{3-[(tert-butyldimethylsilyl)oxy]-1-(methanesulfonyloxy)propyl}-2-oxopyrrolidine-1-carboxylate

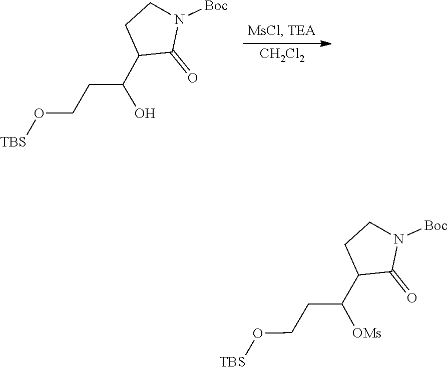

To a mixture of tert-butyl 3-{3-[(tert-butyldimethylsilyl)oxy]-1-hydroxypropyl}-2-oxopyrrolidine-1-carboxylate (11.7 g, 31.32 mmol) and TEA (9.5 g, 93.96 mmol) in CH₂Cl₂ (250.0 mL) was added MsCl (5.4 g, 46.98 mmol) at 0° C. The mixture was stirred at room temperature for 16 h. After the reaction was completed, the mixture was diluted with H₂O and extracted with CH₂Cl₂. The combined organic layer was washed with brine, dried over anhydrous sodium sulfate and filtered. The filtrate was concentrated under vacuum to afford tert-butyl 3-{3-[(tert-butyldimethylsilyl)oxy]-1-(methanesulfonyloxy)propyl}-2-oxopyrrolidine-1-carboxylate (10.0 g, crude) as a light yellow oil. LCMS (ESI, m/z): [M+H]⁺=452.2.

### Step 3. Synthesis of tert-butyl (3E)-3-{3-[(tert-butyldimethylsilyl)oxy]propylidene}-2-oxopyrrolidine-1-carboxylate

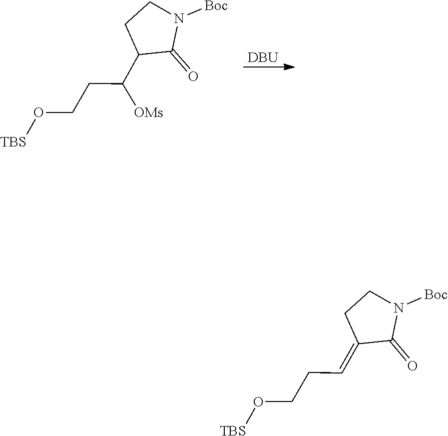

To a solution of tert-butyl 3-{3-[(tert-butyldimethylsilyl)oxy]-1-(methanesulfonyloxy)propyl}-2-oxopyrrolidine-1-carboxylate (10.0 g, crude) in CH₂Cl₂ (80.0 mL) was added DBU (7.8 g, 30.99 mmol) at 0° C. The mixture was stirred at room temperature for 3 h. After the reaction was completed, the mixture was diluted with H₂O and extracted with CH₂Cl₂. The combined organic layer was washed with brine, dried over anhydrous sodium sulfate and filtered. The filtrate was evaporated in vacuo. The residue was purified by flash column chromatography with petroleum ether/EtOAc (3/7, v/v) to afford tert-butyl (3E)-3-{3-[(tert-butyldimethylsilyl)oxy]propylidene}-2-oxopyrrolidine-1-carboxylate (3.0 g, 38%) as a colorless oil. LCMS (ESI, m/z): [M+H]⁺=356.0.

### Step 4. Synthesis of (3E)-3-(3-hydroxypropylidene)pyrrolidin-2-one

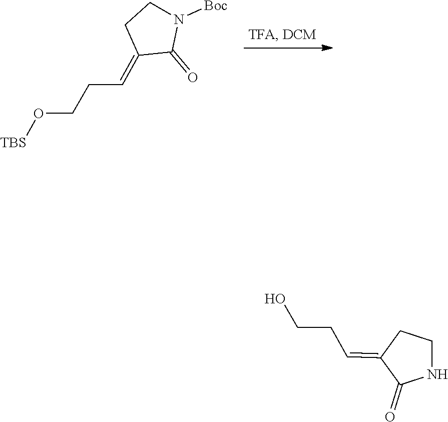

A mixture of tert-butyl (3E)-3-{3-[(tert-butyldimethylsilyl)oxy]propylidene}-2-oxopyrrolidine-1-carboxylate (3.3 g, 9.28 mmol) and TFA (3.0 mL) in DCM (15.0 mL) was stirred at room temperature for 1 h. The mixture was acidified to pH=7 with saturated Na₂CO₃ (aq.). The resulting mixture was extracted with CH₂Cl₂. The combined organic layers were washed with water and dried over anhydrous Na₂SO₄. After filtration, the filtrate was concentrated under reduced pressure to afford (3E)-3-(3-hydroxypropylidene)pyrrolidin-2-one (1.0 g, crude) as a light yellow solid. LCMS (ESI, m/z): [M+H]⁺=142.0.

### Step 5. Synthesis of (3E)-3-{3-[(tert-butyldimethylsilyl)oxy]propylidene}pyrrolidin-2-one

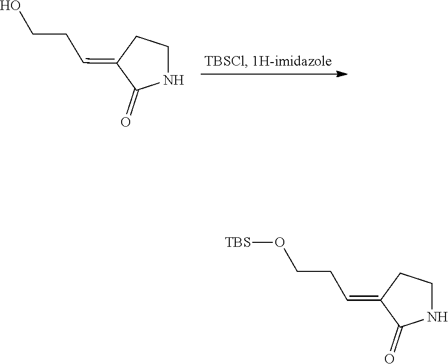

A mixture of (3Z)-3-(3-hydroxypropylidene)pyrrolidin-2-one (1.0 g, crude), imidazole (1.3 g, 51.43 mmol) and TBSCl (3.7 g, 24.82 mmol) in DMF (10.0 mL) was stirred at room temperature for 16 h. After the reaction was completed, the resulting mixture was diluted with H₂O and extracted with EtOAc. The combined organic layers were washed with water, dried over anhydrous Na₂SO₄ and filtered. The filtrate was concentrated under reduced pressure. The residue was purified by flash column chromatography with CH₂Cl₂/MeOH (9/1, v/v) to afford (3E)-3-{3-[(tert-butyldimethylsilyl)oxy]propylidene}pyrrolidin-2-one (1.4 g, 77%) as a colorless oil. LCMS (ESI, m/z): [M+H]⁺=256.2.

### Step 6. Synthesis of (3E)-3-{3-[(tert-butyldimethylsilyl)oxy]propylidene}-1-{4-[(3-methyl-4-{[1,2,4]triazolo[1,5-a]pyridin-7-yloxy}phenyl)amino]quinazolin-6-yl}pyrrolidin-2-one

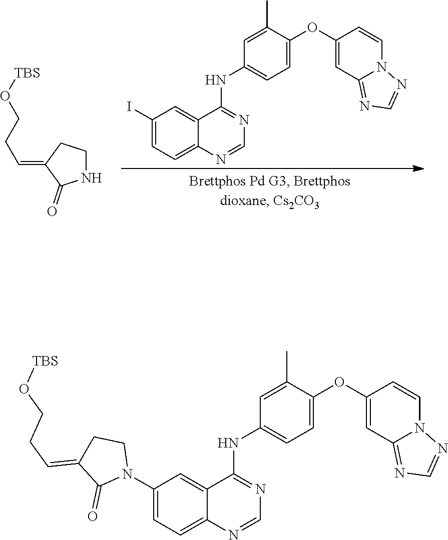

To a stirred mixture of (3E)-3-{3-[(tert-butyldimethylsilyl)oxy]propylidene}pyrrolidin-2-one (852.6 mg, 3.33 mmol), 6-iodo-N-(3-methyl-4-{[1,2,4]triazolo[1,5-a]pyridin-7-yloxy}phenyl)quinazolin-4-amine (1.5 g, 3.03 mmol) and Cs₂CO₃ (2.9 g, 9.10 mmol) in 1,4-dioxane (10.0 mL) was added BrettPhos (651.5 mg, 1.21 mmol) and BrettPhos Pd G3 (550.1 mg, 0.60 mmol) at room temperature under N₂. The mixture was stirred at 100° C. for 2 h. After the reaction was completed, the mixture was filtered. The filtrate was concentrated under reduced pressure. The residue was purified by flash column chromatography with CH₂Cl₂/MeOH (9/1, v/v) to afford (3E)-3-{3-[(tert-butyldimethylsilyl)oxy]propylidene}-1-{4-[(3-methyl-4-{[1,2,4]triazolo[1,5-a]pyridin-7-yloxy}phenyl)amino]quinazolin-6-yl}pyrrolidin-2-one (920.0 mg, 48%) as a yellow solid. LCMS (ESI, m/z): [M+H]⁺=622.3.

### Step 7. Synthesis of (3E)-3-(3-hydroxypropylidene)-1-{4-[(3-methyl-4-{[1,2,4]triazolo[1,5-a]pyridin-7-yloxy}phenyl)amino]quinazolin-6-yl}pyrrolidin-2-one

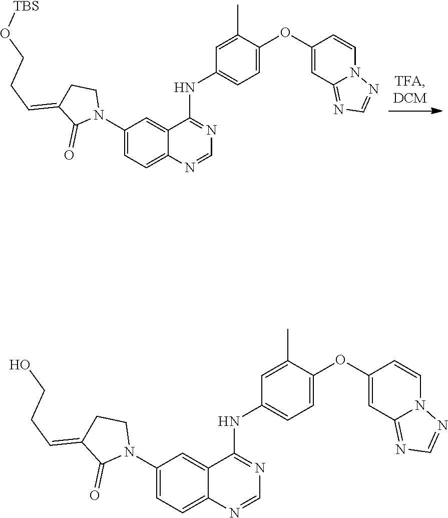

A mixture of (3E)-3-{3-[(tert-butyldimethylsilyl)oxy]propylidene}-1-{4-[(3-methyl-4-{[1,2,4]triazolo[1,5-a]pyridin-7-yloxy}phenyl)amino]quinazolin-6-yl}pyrrolidin-2-one (920.0 mg, 1.48 mmol) and TFA (3.0 mL) in DCM (6.0 mL) was stirred at room temperature for 1 h. After the reaction was completed, the mixture was acidified to pH=7 with saturated Na₂CO₃ (aq.). The resulting mixture was extracted with CH₂Cl₂. The combined organic layers were washed with brine, dried over anhydrous Na₂SO₄ and filtered. The filtrate was concentrated under reduced pressure. The residue was purified by flash column chromatography with CH₂Cl₂/MeOH (9/1, v/v) to afford (3E)-3-(3-hydroxypropylidene)-1-{4-[(3-methyl-4-{[1,2,4]triazolo[1,5-a]pyridin-7-yloxy}phenyl)amino]quinazolin-6-yl}pyrrolidin-2-one (600.0 mg, 79%) as a yellow solid. LCMS (ESI, m/z): [M+H]⁺=508.2.

### Step 8. Synthesis of 3-[(3E)-1-{4-[(3-methyl-4-{[1,2,4]triazolo[1,5-a]pyridin-7-yloxy}phenyl)amino]quinazolin-6-yl}-2-oxopyrrolidin-3-ylidene]propyl 4-methylbenzenesulfonate

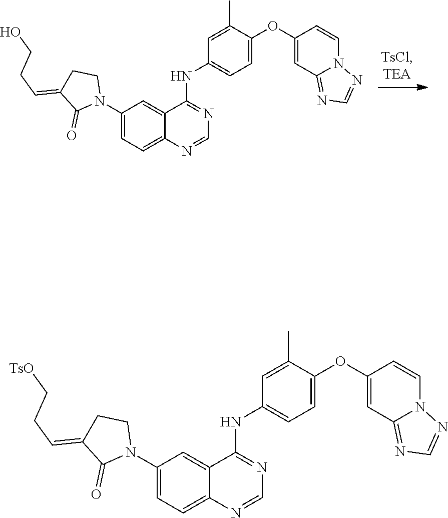 [SMILES: Cc1cc(Nc2ncnc3ccc(N4CC/C(=C\CCO)C4=O)cc23)ccc1Oc1ccn2ncnc2c1 ; Cc1ccc(S(=O)(=O)OCC/C=C2\CCN(c3ccc4ncnc(Nc5ccc(Oc6ccn7ncnc7c6)c(C)c5)c4c3)C2=O)cc1]

To a mixture of (3E)-3-(3-hydroxypropylidene)-1-{4-[(3-methyl-4-{[1,2,4]triazolo[1,5-a]pyridin-7-yloxy}phenyl)amino]quinazolin-6-yl}pyrrolidin-2-one (300.0 mg, 0.59 mmol) and triethylamine (179.4 mg, 1.77 mmol) in DCM (10.0 mL) was added 4-dimethylaminopyridine (14.4 mg, 0.11 mmol) and TsCl (169.0 mg, 0.88 mmol) at room temperature. The resulting mixture was stirred at room temperature for 16 h. After the reaction was completed, the resulting mixture was concentrated under reduced pressure. The residue was purified by flash column chromatography with ethyl acetate/methanol (5/1, v/v) to afford 3 [(3E)-1-{4-[(3-methyl-4-{[1,2,4]triazolo[1,5-a]pyridin-7-yloxy}phenyl)amino]quinazolin-6-yl}-2-oxopyrrolidin-3-ylidene]propyl 4-methylbenzenesulfonate (300.0 mg, 76%) as a yellow oil. LCMS (ESI, m/z): [M+H]⁺=662.2.

### Step 9. Synthesis of (3E)-1-{4-[(3-methyl-4-{[1,2,4]triazolo[1,5-a]pyridin-7-yloxy}phenyl)amino]quinazolin-6-yl}-3-[3-(4-methylpiperazin-1-yl)propylidene]pyrrolidin-2-one (Compound 40)

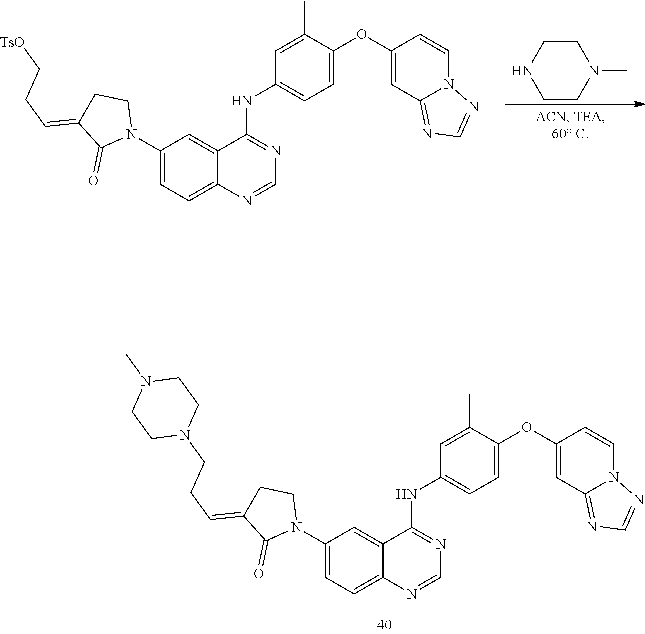

To a mixture of 3-[(3E)-1-{4-[(3-methyl-4-{[1,2,4]triazolo[1,5-a]pyridin-7-yloxy}phenyl)amino]quinazolin-6-yl}-2-oxopyrrolidin-3-ylidene]propyl 4-methylbenzenesulfonate (150.0 mg, 0.22 mmol) and triethylamine (68.8 mg, 0.68 mmol) in ACN (5.0 mL) was added 1-methylpiperazine (68.1 mg, 0.68 mmol) at room temperature. The resulting mixture was stirred at 60° C. for 16 h. After the reaction was completed, the resulting mixture was concentrated under reduced pressure. The residue was purified by reverse phase flash column chromatography with CH₃CN/H₂O (6/4, v/v) and then purified by Prep-HPLC with the following conditions: (Column: YMC-Actus Triart Diol-HICK, 2×25 cm, 5 μm; Mobile Phase A: CO₂, Mobile Phase B: MeOH (0.5% 2M NH₃-MeOH)—HPLC; Flow rate: 75 mL/min; Gradient: isocratic 30% B; Column Temperature(° C.): 35; Wave Length: 254 nm) to afford (3E) 1-{4-[(3-methyl-4-{[1,2,4]triazolo[1,5-a]pyridin-7-yloxy}phenyl)amino]quinazolin-6-yl}-3-[3-(4-methylpiperazin-1-yl)propylidene]pyrrolidin-2-one (Compound 40) (4.6 mg, 3%) as a white solid. LCMS (ESI, m/z): [M+H]⁺=590.3. ¹H NMR (400 MHz, DMSO-d₆): δ 9.86 (s, 1H), 8.94 (d, J=7.2 Hz, 1H), 8.75-8.72 (m, 1H), 8.58 (s, 1H), 8.39 (s, 1H), 8.33 (s, 1H), 7.85-7.81 (m, 3H), 7.24 (d, J=9.2 Hz, 1H), 7.05-7.03 (m, 1H), 6.82 (d, J=2.4 Hz, 1H), 6.53-6.49 (m, 1H), 4.09-4.06 (m, 2H), 2.93-2.89 (m, 2H), 2.41-2.33 (m, 2H), 2.28 (s, 3H), 2.22 (s, 3H).

### Example S39: Synthesis of (R)-4-methyl-1-(4-((3-methyl-4-((1-methyl-1H-benzo[d]imidazol-5-yl)oxy)phenyl)amino)quinazolin-6 yl)-3-methylenepyrrolidin-2-one (Compound 41)

### Step 1. Synthesis of (R)-4-methyl-1-(4-((3-methyl-4-((1-methyl-1H-benzo[d]imidazol-5-yl)oxy)phenyl)amino)quinazolin-6-yl)-3-methylenepyrrolidin-2-one (Compound 41)

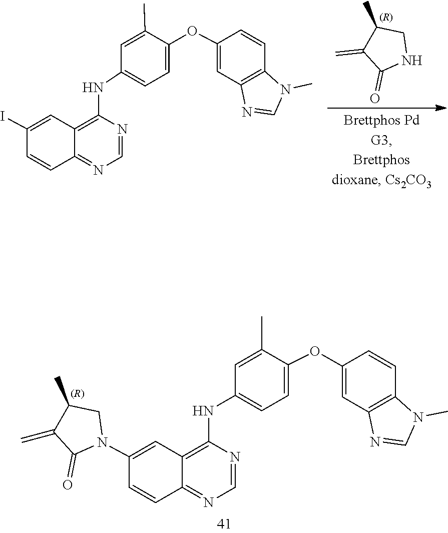

To a solution of 6-iodo-N-{3-methyl-4-[(1-methyl-1,3-benzodiazol-5-yl)oxy]phenyl}quinazolin-4-amine (200.0 mg, 0.39 mmol) in 1,4-dioxane (5.0 mL) was added (4R)-4-methyl-3-methylidenepyrrolidin-2-one (65.7 mg, 0.59 mmol), Cs₂CO₃ (453.3 mg, 1.17 mmol), BrettPhos Pd G3 (71.4 mg, 0.07 mmol) and BrettPhos (84.6 mg, 0.15 mmol) at room temperature under N₂. The resulting mixture was stirred at 100° C. for 16 h. After the reaction was completed, the mixture was concentrated under reduced pressure. The residue was purified by flash chromatography with CH₂Cl₂/MeOH (90/10, v/v) and then purified by Prep-HPLC with the following conditions: (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 35% B to 55% B in 8 min; 254 nm) to afford (R)-4-methyl-1-(4-((3-methyl-4-((1-methyl-1H-benzo[d]imidazol-5-yl)oxy)phenyl)amino)quinazolin-6-yl)-3-methylenepyrrolidin-2-one (Compound 41) (10.1 mg, 6%) as a yellow solid. LCMS (ESI, m/z): [M+H]⁺=491.3. ¹H NMR (400 MHz, DMSO-d₆): δ 9.79 (s, 1H), 8.72-8.69 (m, 1H), 8.53 (s, 1H), 8.28 (d, J=2.0 Hz, 1H), 8.18 (s, 1H), 7.81 (d, J=9.2 Hz, 1H), 7.69 (d, J=2.0 Hz, 1H), 7.61-7.57 (m, 2H), 7.11 (d, J=2.0 Hz, 1H), 7.03-7.00 (m, 1H), 6.88 (d, J=8.8 Hz, 1H), 5.99 (d, J=2.8 Hz, 1H), 5.55 (d, J=2.0 Hz, 1H), 4.23-4.18 (m, 1H), 3.84 (s, 3H), 3.66-3.62 (m, 1H), 3.20-3.15 (m, 1H), 2.27 (s, 3H), 1.34 (d, J=6.8 Hz, 3H).

### Example S40: Synthesis of (1R,5R)-2-{4-[(3-methyl-4-{[1,2,4]triazolo[1,5-a]pyridin-7-yloxy}phenyl)amino]pyrido[3,4-d]pyrimidin-6-yl}-4-methylidene-2-azabicyclo[3.1.0]hexan-3-one and (1S,5S)-2-{4-[(3-methyl-4-[[1,2,4]triazolo[1,5-a]pyridin-7-yloxyphenyl)amino]pyrido[3,4-d]pyrimidin-6-yl}-4-methylidene-2-azabicyclo[3.1.0]hexan-3-one (Compounds 42 and 43)

### Step 1. Synthesis of 2-{4-[(3-methyl-4-{[1,2,4]triazolo[1,5-a]pyridin-7-yloxy}phenyl)amino]pyrido[3,4-d]pyrimidin-6-yl}-4-methylidene-2-azabicyclo[3.1.0]hexan-3-one

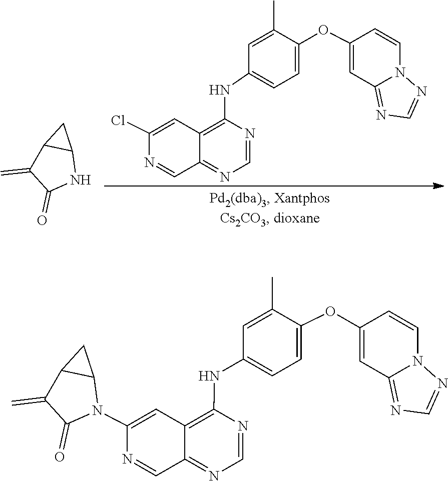

To a solution of 6-chloro-N-(3-methyl-4-{[1,2,4]triazolo[1,5-a]pyridin-7-yloxy}phenyl)pyrido[3,4-d]pyrimidin-4-amine (400.0 mg, 0.99 mmol) in dioxane (30.0 mL) was added 4-methylidene-2-azabicyclo[3.1.0]hexan-3-one (324.3 mg, 2.97 mmol), Cs₂CO₃ (968.2 mg, 2.97 mmol), Pd₂(dba)₃ (181.4 mg, 0.20 mmol) and XantPhos (114.6 mg, 0.20 mmol) at room temperature under N₂. The resulting mixture was stirred at 100° C. for 16 h. After the reaction was completed, the resulting mixture was filtered. The filtrate was concentrated under reduced pressure. The residue was purified by flash chromatography with dichloromethane/methanol (90/10, v/v) to afford 2-{4-[(3-methyl-4-{[1,2,4]triazolo[1,5-a]pyridin-7-yloxy}phenyl)amino]pyrido[3,4-d]pyrimidin-6-yl}-4-methylidene-2-azabicyclo[3.1.0]hexan-3-one (90.0 mg, 19%) as a white solid. LCMS (ESI, m/z): [M+H]⁺=477.2.

### Step 2. Synthesis of (1R,5R)-2-{4-[(3-methyl-4-{[1,2,4]triazolo[1,5-a]pyridin-7-yloxy}phenyl)amino]pyrido[3,4-d]pyrimidin-6-yl}-4-methylidene-2-azabicyclo[3.1.0]hexan-3-one and (1S,5S)-2-{4-[(3-methyl-4-{[1,2,4]triazolo[1,5-a]pyridin-7-yloxy}phenyl)amino]pyrido[3,4-d]pyrimidin-6-yl}-4-methylidene-2-azabicyclo[3.1.0]hexan-3-one (Compounds 42 and 43)

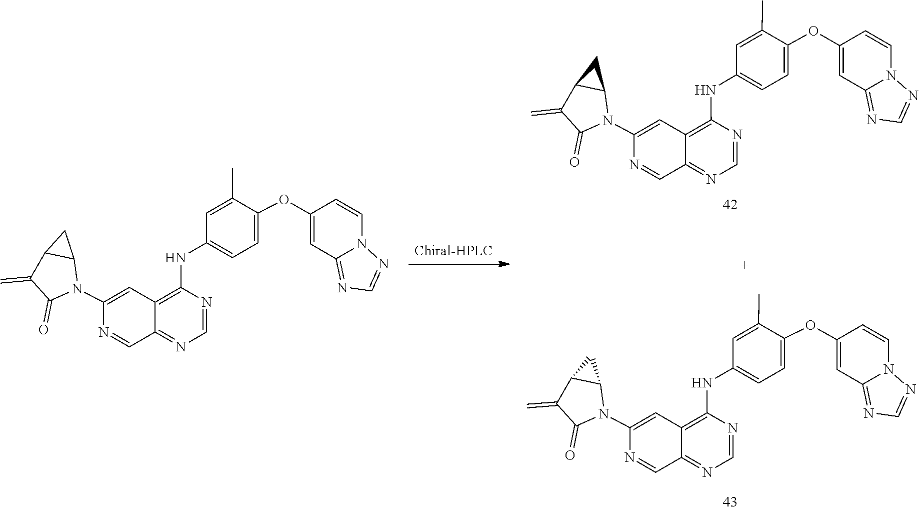 [SMILES: C=C1C(=O)N(c2cc3c(Nc4ccc(Oc5ccn6ncnc6c5)c(C)c4)ncnc3cn2)C2CC12 ; C=C1C(=O)N(c2cc3c(Nc4ccc(Oc5ccn6ncnc6c5)c(C)c4)ncnc3cn2)[C@H]2C[C@@H]12 ; C=C1C(=O)N(c2cc3c(Nc4ccc(Oc5ccn6ncnc6c5)c(C)c4)ncnc3cn2)[C@@H]2C[C@H]12]

The racemic mixture of 2-{4-[(3-methyl-4-{[1,2,4]triazolo[1,5-a]pyridin-7-yloxy}phenyl)amino]pyrido[3,4-d]pyrimidin-6-yl}-4-methylidene-2-azabicyclo[3.1.0]hexan-3-one (90.0 mg, 0.19 mmol) was separated by Prep-Chiral-HPLC with the following conditions (Column: CHIRAL ART Amylose-SA, 2×25 cm, 5 μm; Mobile Phase A: Hex (0.5% 2M NH₃-MeOH)-HPLC, Mobile Phase B: MeOH: EtOH=1: 1-HPLC; Flow rate: 20 mL/min; Gradient: 60% B to 60% B in 20 min; Wave Length: 220/254 nm; RT1(min): 12.02; RT2(min): 16.10) to afford 2-{4-[(3-methyl-4-{[1,2,4]triazolo[1,5-a]pyridin-7-yloxy}phenyl)amino]pyrido[3,4-d]pyrimidin-6-yl}-4-methylidene-2-azabicyclo[3.1.0]hexan-3-one Enantiomer 1 (Retention time: 12.02 min, 10.4 mg, 23%) as a white solid and 2-{4-[(3-methyl-4-{[1,2,4]triazolo[1,5-a]pyridin-7-yloxy}phenyl)amino]pyrido[3,4-d]pyrimidin-6-yl}-4-methylidene-2-azabicyclo[3.1.0]hexan-3-one Enantiomer 2 (Retention time: 16.10 min, 11.0 mg, 24%) as a white solid. The absolute stereochemistry of Enantiomers 1 and 2 was not assigned. The two enantiomeric structures that could be obtained from chiral separation of the enantiomeric mixture as described above are shown as Compounds 42 and 43 in Table 1.

2-{4-[(3-methyl-4-{[1,2,4]triazolo[1,5-a]pyridin-7-yloxy}phenyl)amino]pyrido[3,4-d]pyrimidin-6-yl}-4-methylidene-2-azabicyclo[3.1.0]hexan-3-one Enantiomer 1: RT1(min): 12.02; LCMS (ESI, m/z): [M+H]⁺=477.2. ¹H NMR (400 MHz, DMSO-d₆): δ 10.33 (s, 1H), 9.13 (s, 1H), 9.05 (s, 1H), 8.94 (d, J=7.2 Hz, 1H), 8.66 (s, 1H), 8.39 (s, 1H), 7.86-7.81 (m, 2H), 7.23 (d, J=8.8 Hz, 1H), 7.05-7.03 (m, 1H), 6.83 (d, J=2.0 Hz, 1H), 6.06 (s, 1H), 5.78 (s, 1H), 4.33-4.29 (m, 1H), 2.21 (s, 3H), 1.45-1.43 (m, 1H), 0.87-0.82 (m, 1H).

2-{4-[(3-methyl-4-{[1,2,4]triazolo[1,5-a]pyridin-7-yloxy}phenyl)amino]pyrido[3,4-d]pyrimidin-6-yl}-4-methylidene-2-azabicyclo[3.1.0]hexan-3-one Enantiomer 2: RT2(min): 16.10; LCMS (ESI, m/z): [M+H]⁺=477.2. ¹H NMR (400 MHz, DMSO-d₆): δ 10.32 (s, 1H), 9.14-8.93 (m, 3H), 8.66 (s, 1H), 8.39 (s, 1H), 7.86-7.82 (m, 2H), 7.24 (d, J=8.4 Hz, 1H), 7.05-7.03 (m, 1H), 6.83 (s, 1H), 6.06 (s, 1H), 5.78 (s, 1H), 4.35-4.29 (m, 1H), 2.22 (s, 3H), 1.45-1.43 (m, 1H), 0.87-0.81 (m, 1H).

### Example S41: Synthesis of (3E)-3 [3-(dimethylamino)propylidene]-1-[4-({3-methyl-4[(1-methyl-1,3-benzodiazol-5 yl)oxy]phenyl}amino)quinazolin-6 yl]pyrrolidin-2-one (Compound 44)

### Step 1. Synthesis of 6-iodo-N-{3-methyl-4-[(1-methyl-1,3-benzodiazol-5-yl)oxy]phenyl}quinazolin-4-amine

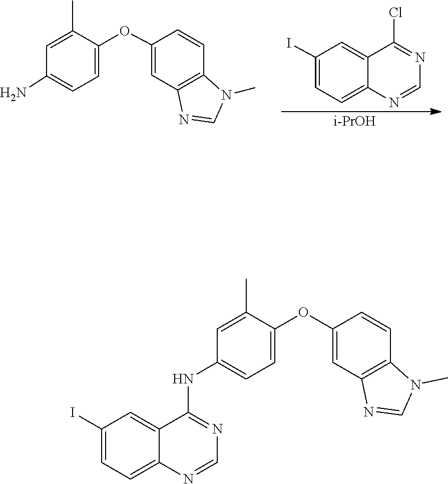 [SMILES: "i-PrOH" ; Cc1cc(N)ccc1Oc1ccc2c(c1)ncn2C ; Clc1ncnc2ccc(I)cc12 ; Cc1cc(Nc2ncnc3ccc(I)cc23)ccc1Oc1ccc2c(c1)ncn2C]

To a solution of 3-methyl-4-[(1-methyl-1,3-benzodiazol-5-yl)oxy]aniline (500.0 mg, 1.97 mmol) in i-PrOH (5.0 mL) was added 4-chloro-6-iodoquinazoline (573.4 mg, 1.97 mmol) at 0° C. The resulting mixture was stirred at room temperature for 2 h. After the reaction was completed, the resulting mixture was concentrated under reduced pressure. The residue was purified by flash column chromatography with CH₂Cl₂/CH₃OH (5/1, v/v) to afford 6-iodo-N-{3-methyl-4-[(1-methyl-1,3-benzodiazol-5-yl)oxy]phenyl}quinazolin-4-amine (600.0 mg, 59%) as a yellow solid. LCMS (ESI, m/z): [M+H]⁺=508.1.

### Step 2. Synthesis of (3E)-3-[3-(dimethylamino)propylidene]-1-[4-({3-methyl-4-[(1-methyl-1,3-benzodiazol-5-yl)oxy]phenyl}amino)quinazolin-6-yl]pyrrolidin-2-one (Compound 44)

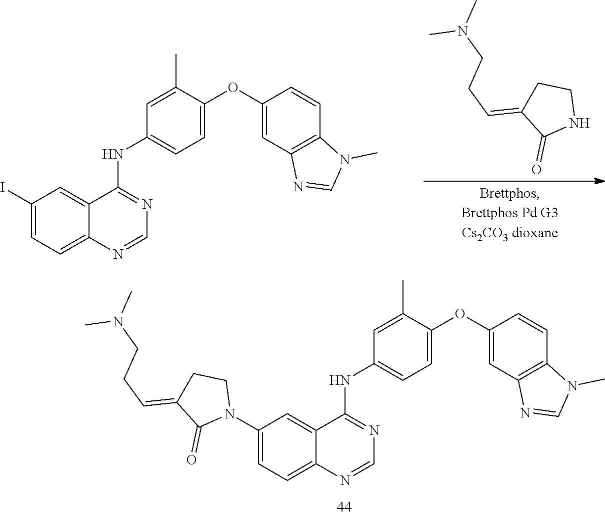

To a solution of 6-iodo-N-{3-methyl-4-[(1-methyl-1,3-benzodiazol-5-yl)oxy]phenyl}quinazolin-4-amine (200.0 mg, 0.39 mmol) in 1,4-dioxane (3.0 mL) were added (3E)-3-[3-(dimethylamino)propylidene]pyrrolidin-2-one (66.3 mg, 0.39 mmol), Cs₂CO₃ (385.3 mg, 1.18 mmol), BrettPhos Pd G3 (35.7 mg, 0.04 mmol) and BrettPhos (42.3 mg, 0.08 mmol) at room temperature under N₂. The mixture was stirred at 100° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) and then purified by Prep-HPLC with the following conditions: (Column: YMC-Actus Triart C18 ExRS, 30×150 mm, 5 μm; Mobile Phase A: Water (10 mmol/L NH₄HCO₃), Mobile Phase B: ACN; Flow rate: 60 mL/min; Gradient: 35% B to 35% B in 8 min; Wave Length: 254 nm) to afford (3E)-3-[3-(dimethylamino)propylidene]-1-[4-({3-methyl-4-[(1-methyl-1,3-benzodiazol-5-yl)oxy]phenyl}amino)quinazolin-6-yl]pyrrolidin-2-one (Compound 44) (5.3 mg, 2%) as a write solid. LCMS (ESI, m/z): [M+H]⁺=548.3. ¹H NMR (400 MHz, DMSO-d₆): δ 9.76 (s, 1H), 8.72 (d, J=9.2 Hz, 1H), 8.53 (s, 1H), 8.28 (s, 1H), 8.17 (s, 1H), 7.80 (d, J=9.2 Hz, 1H), 7.70 (s, 1H), 7.61-7.57 (m, 2H), 7.12 (s, 1H), 7.02 (d, J=8.8 Hz, 1H), 6.89 (d, J=8.4 Hz, 1H), 6.51 (d, J=6.8 Hz, 1H), 4.07-4.04 (m, 2H), 3.85 (s, 3H), 2.92-2.87 (m, 2H), 2.41-2.34 (m, 4H), 2.27 (s, 3H), 2.18 (s, 6H).

### Example S42: Synthesis of (E)-3-(2-(dimethylamino)ethylidene)-1-(4-((3-methyl-4-((1-methyl-1H-benzo[d]imidazol-5 yl)oxy)phenyl)amino)pyrido[3,4-d]pyrimidin-6 yl)pyrrolidin-2-one (Compound 45)

### Step 1. Synthesis of (E)-3-(2-(dimethylamino)ethylidene)-1-(4-((3-methyl-4-((1-methyl-1H-benzo[d]imidazol-5-yl)oxy)phenyl)amino)pyrido[3,4-d]pyrimidin-6-yl)pyrrolidin-2-one (Compound 45)

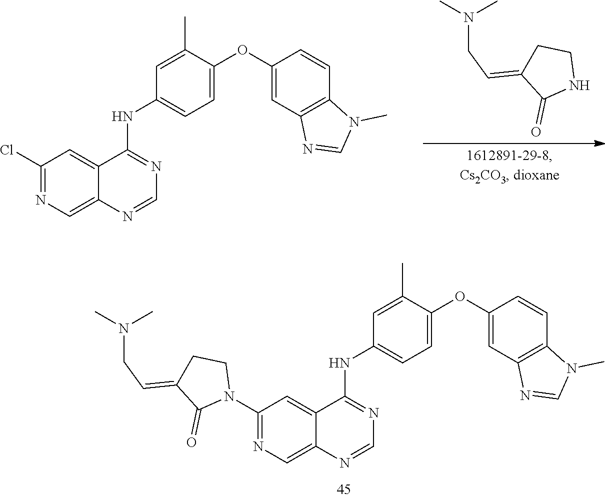

To a solution of 6-chloro-N-(3-methyl-4-((1-methyl-1H-benzo[d]imidazol-5-yl)oxy)phenyl)pyrido[3,4-d]pyrimidin-4-amine (100.0 mg, 0.24 mmol) in dioxane (3.0 mL) was added (E)-3-(2-(dimethylamino)ethylidene)pyrrolidin-2-one (55.5 mg, 0.36 mmol), Cs₂CO₃ (234.5 mg, 0.72 mmol) and Pd-PEPPSI-IPentCl 2-methylpyridine (o-picoline) (20.2 mg, 0.02 mmol) at room temperature under N₂. The resulting mixture was stirred at 100° C. for 2 h. After the reaction was completed, the resulting mixture was concentrated under reduced pressure. The residue was purified by flash chromatography with DCM/MeOH (10/1, v/v) and then purified by Prep-HPLC with the following conditions Column: (Column: Xselect CSH C18 OBD Column 30×150 mm, 5 μm; Mobile Phase A: Water (0.1% FA), Mobile Phase B: ACN; Flow rate: 60 mL/min; Gradient: 7% B to 20% B in 12 min; Wave Length: 254 nm) to afford (E)-3-(2-(dimethylamino)ethylidene)-1-(4-((3-methyl-4-((1-methyl-1H-benzo[d]imidazol-5-yl)oxy)phenyl)amino)pyrido[3,4-d]pyrimidin-6-yl)pyrrolidin-2-one (Compound 45) (3.9 mg, 3%) as a yellow solid. LCMS (ESI, m/z): [M+H]⁺=535.3. ¹H NMR (400 MHz, DMSO-d₆): δ 10.25 (s, 1H), 9.18-8.96 (m, 2H), 8.59-8.49 (m, 1H), 8.17 (s, 1H), 7.73-7.56 (m, 3H), 7.13-7.00 (m, 2H), 6.85-6.78 (m, 1H), 6.52-6.37 (m, 1H), 4.14-4.03 (m, 2H), 3.90-3.82 (m, 3H), 3.21-2.95 (m, 2H), 2.89-2.75 (m, 2H), 2.34-2.16 (m, 9H).

### Example S43: Synthesis of 1-(4-((3-methyl-4-(quinoxalin-6-yloxy)phenyl)amino)quinazolin-6 yl)-3-methylenepyrrolidin-2-one (Compound 46)

### Step 1. Synthesis of 6-iodo-N-(3-methyl-4-(quinoxalin-6-yloxy)phenyl)quinazolin-4-amine

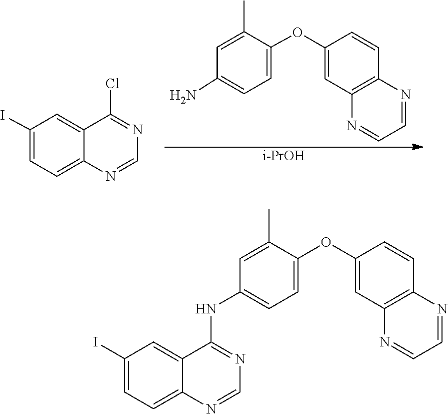 [SMILES: "i-PrOH" ; Clc1ncnc2ccc(I)cc12 ; Cc1cc(N)ccc1Oc1ccc2nccnc2c1 ; Cc1cc(Nc2ncnc3ccc(I)cc23)ccc1Oc1ccc2nccnc2c1]

To a solution of 4-chloro-6-iodoquinazoline (300.0 mg, 1.03 mmol) in i-PrOH (5.0 mL) was added 3-methyl-4-(quinoxalin-6-yloxy)aniline (259.5 mg, 1.03 mmol) at room temperature. The resulting mixture was stirred at room temperature for 1 h. After the reaction was completed, the resulting mixture was concentrated under reduced pressure. The residue was purified by flash column chromatography with dichloromethane/methanol (10/1, v/v) to afford 6-iodo-N-(3-methyl-4-(quinoxalin-6-yloxy)phenyl)quinazolin-4-amine (510.0 mg, 97%) as a yellow solid. LCMS (ESI, m/z): [M+H]⁺=506.0.

### Step 2. Synthesis of 1-(4-((3-methyl-4-(quinoxalin-6-yloxy)phenyl)amino)quinazolin-6-yl)-3-methylenepyrrolidin-2-one (Compound 46)

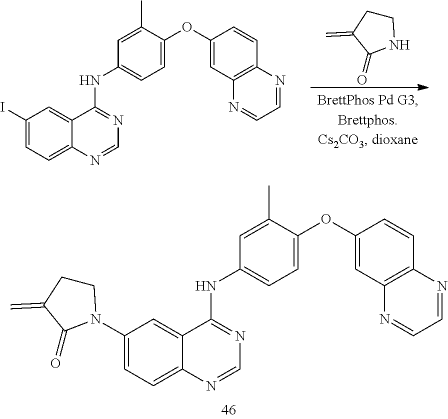

To a solution of 6-iodo-N-(3-methyl-4-(quinoxalin-6-yloxy)phenyl)quinazolin-4-amine (100.0 mg, 0.20 mmol) in 1,4-dioxane (5.0 mL) was added 3-methylenepyrrolidin-2-one (57.7 mg, 0.59 mmol), Cs₂CO₃ (322.4 mg, 0.99 mmol), BrettPhos (21.2 mg, 0.04 mmol) and BrettPhos Pd G3 (17.9 mg, 0.02 mmol) at room temperature under N₂. The resulting mixture was stirred at 100° C. for 16 h. After the reaction was completed, the resulting mixture was concentrated under reduced pressure. The residue was purified by flash column chromatography with dichloromethane/methanol (10/1, v/v) and then purified by Prep-HPLC with the following conditions (Column: XBridge Prep Phenyl OBD Column, 19×250 mm, 5 μm; Mobile Phase A: Water (10 mmol/L NH₄HCO₃), Mobile Phase B: ACN; Flow rate: 25 mL/min; Gradient: 42% B to 42% B in 15 min; Wave Length: 254 nm) to afford 1-(4-((3-methyl-4-(quinoxalin-6-yloxy)phenyl)amino)quinazolin-6-yl)-3-methylenepyrrolidin-2-one (Compound 46) (9.0 mg, 9%) as a white solid. LCMS (ESI, m/z): [M+H]⁺=475.3. ¹H NMR (400 MHz, DMSO-d₆): δ 9.88 (s, 1H), 8.88-8.85 (m, 2H), 8.72-8.69 (m, 1H), 8.59 (s, 1H), 8.37 (s, 1H), 8.16 (d, J=9.2 Hz, 1H), 7.86-7.80 (m, 3H), 7.72-7.69 (m, 1H), 7.24 (d, J=8.4 Hz, 1H), 7.15 (d, J=2.8 Hz, 1H), 6.00 (s, 1H), 5.56 (s, 1H), 4.09-4.06 (m, 2H), 3.01-2.98 (m, 2H), 2.27 (s, 3H).

### Example S44: Synthesis of (E)-1-(4-((4-([1,2,4]triazolo[1,5-a]pyridin-7 yloxy)-3-methylphenyl)amino)pyrido[3,2-d]pyrimidin-6-yl)-3-(2-(dimethylamino)ethylidene)pyrrolidin-2-one (Compound 47)

### Step 1. Synthesis of (E)-1-(4-((4-([1,2,4]triazolo[1,5-a]pyridin-7-yloxy)-3-methylphenyl)amino)pyrido[3,2-d]pyrimidin-6-yl)-3-(2-(dimethylamino)ethylidene)pyrrolidin-2-one (Compound 47)

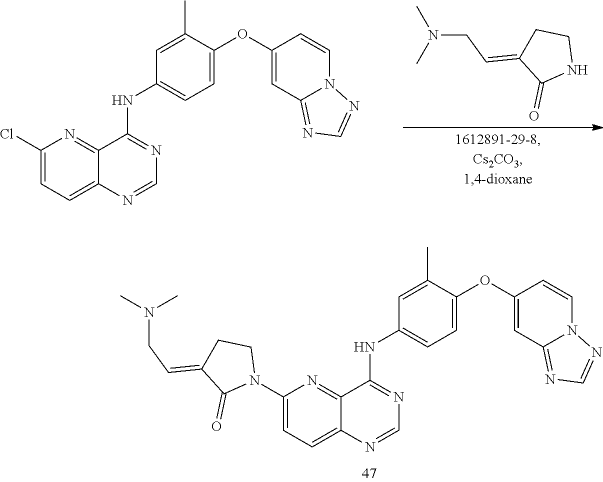 [SMILES: Cc1cc(Nc2ncnc3ccc(Cl)nc23)ccc1Oc1ccn2ncnc2c1 ; CN(C)C/C=C1\CCNC1=O ; Cc1cc(Nc2ncnc3ccc(N4CC/C(=C\CN(C)C)C4=O)nc23)ccc1Oc1ccn2ncnc2c1]

To a solution of N-(4-([1,2,4]triazolo[1,5-a]pyridin-7-yloxy)-3-methylphenyl)-6-chloropyrido[3,2-d]pyrimidin-4-amine (100.0 mg, 0.25 mmol) in dioxane (5.0 mL) was added (E)-3-(2-(dimethylamino)ethylidene)pyrrolidin-2-one (38.2 mg, 0.25 mmol), Cs₂CO₃ (242.1 mg, 0.74 mmol) and Pd-PEPPSI-IPentCl 2-methylpyridine (20.8 mg, 0.03 mmol) at room temperature under N₂. The resulting mixture was stirred at 100° C. for 16 h. After the reaction was completed, the resulting mixture was diluted with water and extracted with EtOAc. The combined organic layers were washed with brine and dried over anhydrous Na₂SO₄. After filtration, the filtrate was concentrated under reduced pressure. The residue was purified by flash column chromatography with DCM/MeOH (9/1, v/v) and then purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 30% B to 40% B in 8 min; Wave Length: 254 nm) to afford (E)-1-(4-((4-([1,2,4]triazolo[1,5-a]pyridin-7-yloxy)-3-methylphenyl)amino)pyrido[3,2-d]pyrimidin-6-yl)-3-(2-(dimethylamino)ethylidene)pyrrolidin-2-one (Compound 47) (2.2 mg, 2%) as a white solid. LCMS (ESI, m/z): [M+H]⁺=522.4. ¹H NMR (400 MHz, DMSO-d₆): δ 9.61 (s, 1H), 9.01-8.94 (m, 2H), 8.64 (s, 1H), 8.39 (s, 1H), 8.28 (d, J=9.2 Hz, 1H), 8.00-7.97 (m, 2H), 7.26 (d, J=8.4 Hz, 1H), 7.05-7.03 (m, 1H), 6.81 (d, J=2.4 Hz, 1H), 6.60-6.56 (m, 1H), 4.38-4.35 (m, 2H), 3.13-3.11 (m, 2H), 2.88-2.70 (m, 2H), 2.23-2.22 (m, 9H).

### Example S45: Synthesis of (3E)-3-[3-(dimethylamino)propylidene]-1-{4-[(3-methyl-4-{[1,2,4]triazolo[1,5-a]pyridin-7-yloxy}phenyl)amino]pyrido[3,2-d]pyrimidin-6-yl}pyrrolidin-2-one (Compound 48)

### Step 1. Synthesis of (3E)-3-[3-(dimethylamino)propylidene]-1-{4-[(3-methyl-4-{[1,2,4]triazolo[1,5-a]pyridin-7-yloxy}phenyl)amino]pyrido[3,2-d]pyrimidin-6-yl}pyrrolidin-2-one (Compound 48)

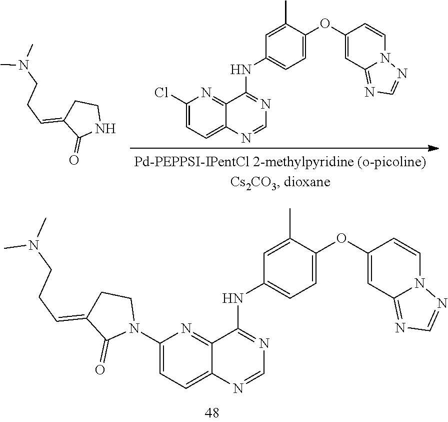 [SMILES: CN(C)CC/C=C1\CCNC1=O ; Cc1cc(Nc2ncnc3ccc(Cl)nc23)ccc1Oc1ccn2ncnc2c1 ; Cc1cc(Nc2ncnc3ccc(N4CC/C(=C\CCN(C)C)C4=O)nc23)ccc1Oc1ccn2ncnc2c1]

To a solution of (3E)-3-[3-(dimethylamino)propylidene]pyrrolidin-2-one (150.0 mg, 0.89 mmol) in dioxane (10.0 mL) was added 6-chloro-N-(3-methyl-4-{[1,2,4]triazolo[1,5-a]pyridin-7-yloxy}phenyl)pyrido[3,2-d]pyrimidin-4-amine (360.0 mg, 0.89 mmol), Cs₂CO₃ (871.5 mg, 2.67 mmol) and Pd-PEPPSI-IPentCl 2-methylpyridine (o-picoline) (75.0 mg, 0.09 mmol) at room temperature under N₂. The resulting mixture was stirred at 100° C. for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions Column: (YMC-Actus Triart C18 ExRS, 30×150 mm, 5 μm; Mobile Phase A: Water (10 mmol/L NH₄HCO₃), Mobile Phase B: ACN; Flow rate: 60 mL/min; Gradient: 35% B to 35% B in 8 min; Wave Length: 254 nm) to afford (3E)-3-[3-(dimethylamino)propylidene]-1-{4-[(3-methyl-4-{[1,2,4]triazolo[1,5-a]pyridin-7-yloxy}phenyl)amino]pyrido[3,2-d]pyrimidin-6-yl}pyrrolidin-2-one (Compound 48) (11.1 mg, 2%) as a white solid. LCMS (ESI, m/z): [M+H]⁺=536.3. ¹H NMR (400 MHz, DMSO-d₆): δ 9.58 (s, 1H), 9.00-8.93 (m, 2H), 8.64 (s, 1H), 8.38 (s, 1H), 8.26 (d, J=9.2 Hz, 1H), 8.00-7.97 (m, 2H), 7.26 (d, J=8.4 Hz, 1H), 7.05-7.03 (m, 1H), 6.81 (d, J=2.4 Hz, 1H), 6.63-6.61 (m, 1H), 4.36-4.33 (m, 2H), 2.89-2.86 (m, 2H), 2.45-2.34 (m, 4H), 2.23 (s, 3H), 2.18 (s, 6H).

### Example S46: Synthesis of (E)-3-(2-(dimethylamino)ethylidene)-1-(4-((3-methyl-4-((6-methylpyridin-3 yl)oxy)phenyl)amino)pyrido[3,2-d]pyrimidin-6-yl)pyrrolidin-2-one (Compound 49)

### Step 1. Synthesis of 2-methyl-5-(2-methyl-4-nitrophenoxy)pyridine

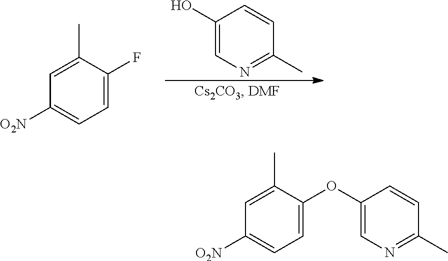

To a solution of 1-fluoro-2-methyl-4-nitrobenzene (1.5 g, 9.67 mmol) in DMF (20.0 mL) was added 6-methylpyridin-3-ol (1.1 g, 10.25 mmol) and Cs₂CO₃ (6.3 g, 19.34 mmol) at room temperature. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 2-methyl-5-(2-methyl-4-nitrophenoxy)pyridine (1.6 g, 67%) as a white solid. LCMS (ESI, m/z): [M+H]⁺=245.1.

### Step 2. Synthesis of 3-methyl-4-((6-methylpyridin-3-yl)oxy)aniline

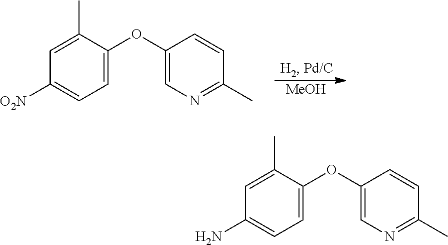

To a solution of 2-methyl-5-(2-methyl-4-nitrophenoxy)pyridine (1.5 g, 6.14 mmol) in CH₃OH (20.0 mL) was added Pd/C (392.1 mg, 10%) at room temperature under N₂. The resulting mixture was stirred at room temperature for 2 h under H₂. After the reaction was completed, the resulting mixture was filtered. The filtrate was concentrated under reduced pressure to afford 3-methyl-4-((6-methylpyridin-3-yl)oxy)aniline (1.3 g, crude) as a yellow solid. LCMS (ESI, m/z): [M+H]⁺=215.1.

### Step 3. Synthesis of 6-chloro-N-(3-methyl-4-((6-methylpyridin-3-yl)oxy)phenyl)pyrido[3,2-d]pyrimidin-4-amine

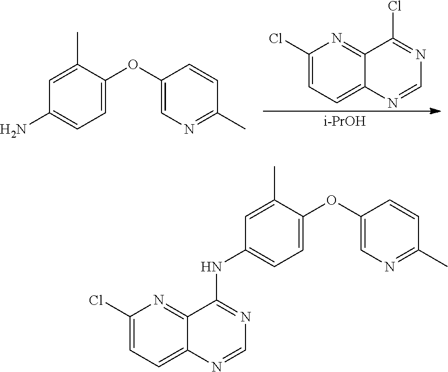 [SMILES: "i-PrOH" ; Cc1ccc(Oc2ccc(N)cc2C)cn1 ; Clc1ccc2ncnc(Cl)c2n1 ; Cc1ccc(Oc2ccc(Nc3ncnc4ccc(Cl)nc34)cc2C)cn1]

To a solution of 3-methyl-4-((6-methylpyridin-3-yl)oxy)aniline (600.0 mg, crude) in i-PrOH (10.0 mL) was added 4,6-dichloropyrido[3,2-d]pyrimidine (590.0 mg, 2.96 mmol) at 0° C. under N₂. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/2, v/v) to afford 6-chloro-N-(3-methyl-4-((6-methylpyridin-3-yl)oxy)phenyl)pyrido[3,2-d]pyrimidin-4-amine (700.0 mg, 66%) as a yellow solid. LCMS (ESI, m/z): [M+H]⁺=378.1.

### Step 4. Synthesis of (E)-3-(2-(dimethylamino)ethylidene)-1-(4-((3-methyl-4-((6-methylpyridin-3-yl)oxy)phenyl)amino)pyrido[3,2-d]pyrimidin-6-yl)pyrrolidin-2-one (Compound 49)

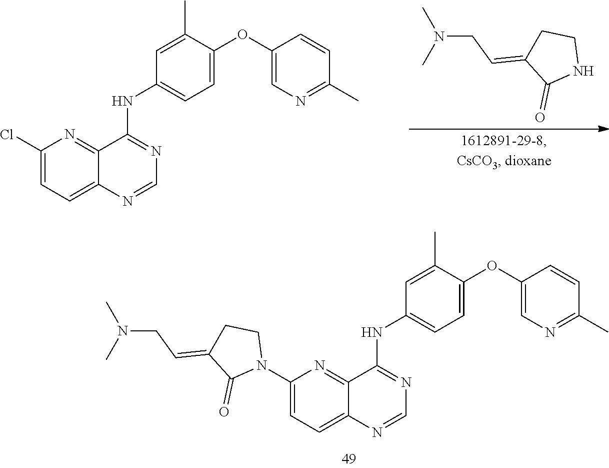

To a solution of 6-chloro-N-(3-methyl-4-((6-methylpyridin-3-yl)oxy)phenyl)pyrido[3,2-d]pyrimidin-4-amine (100.0 mg, 0.27 mmol) in dioxane (5.0 mL) was added (E)-3-(2-(dimethylamino)ethylidene)pyrrolidin-2-one (40.9 mg, 0.27 mmol), Cs₂CO₃ (258.7 mg, 0.80 mmol) and Pd-PEPPSI-IPentCl 2-methylpyridine (o-picoline) (22.3 mg, 0.03 mmol) at room temperature under N₂, The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (5/1, v/v) and then purified by Prep-HPLC with the following conditions (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 35% B to 55% B in 8 min; Wave Length: 254 nm) to afford (E)-3-(2-(dimethylamino)ethylidene)-1-(4-((3-methyl-4-((6-methylpyridin-3-yl)oxy)phenyl)amino)pyrido[3,2-d]pyrimidin-6-yl)pyrrolidin-2-one (Compound (49) (2.0 mg, 2%) as a white solid. LCMS (ESI, m/z): [M+H]⁺=496.3. ¹H NMR (400 MHz, DMSO-d₆): δ 9.53 (s, 1H), 8.97 (d, J=7.6 Hz, 1H), 8.60 (s, 1H), 8.26-8.18 (m, 2H), 7.87-7.84 (m, 2H), 7.27-7.21 (m, 2H), 7.01 (d, J=8.4 Hz, 1H), 6.59-6.56 (m, 1H), 4.37-4.32 (m, 2H), 3.15-3.10 (m, 2H), 2.87-2.82 (m, 2H), 2.45 (s, 3H), 2.24-2.22 (m, 9H).

### Example S47: Synthesis of 1-(4-{[3-methyl-4-(quinazolin-7-yloxy)phenyl]amino}quinazolin-6-yl)-3-methylidenepyrrolidin-2-one (Compound 50)

### Step 1. Synthesis of Quinazolin-7-ol

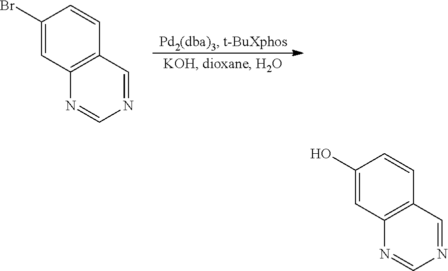 [SMILES: Brc1ccc2cncnc2c1 ; Oc1ccc2cncnc2c1]

To a solution of 7-bromoquinazoline (2.0 g, 9.57 mmol) in 1,4-dioxane (20.0 mL)/H₂O (4.0 mL) were added KOH (2.2 g, 38.27 mmol), t-BuXphos (810.0 mg, 1.91 mmol) and Pd₂(dba)₃ (880.0 mg, 0.96 mmol) at room temperature under N₂. The resulting mixture was stirred at 100° C. for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) to afford quinazolin-7-ol (1.0 g, 71%) as a yellow solid. LCMS (ESI, m/z): [M+H]⁺=147.0.

### Step 2. Synthesis of 7-(2-methyl-4-nitrophenoxy)quinazoline

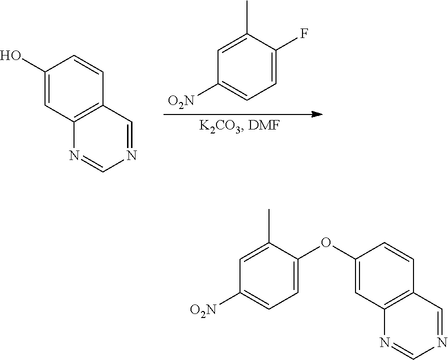 [SMILES: Oc1ccc2cncnc2c1 ; Cc1cc([N+](=O)[O-])ccc1F ; Cc1cc([N+](=O)[O-])ccc1Oc1ccc2cncnc2c1]

To a solution of quinazolin-7-ol (1.0 g, 6.84 mmol) in DMF (10.0 mL) were added 1-fluoro-2-methyl-4-nitrobenzene (1.1 g, 6.84 mmol) and K₂CO₃ (2.8 g, 20.53 mmol) at room temperature. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) to afford 7-(2-methyl-4-nitrophenoxy)quinazoline (500.0 mg, 25%) as a yellow solid. LCMS (ESI, m/z): [M+H]⁺=282.1.

### Step 3. Synthesis of 3-methyl-4-(quinazolin-7-yloxy)aniline

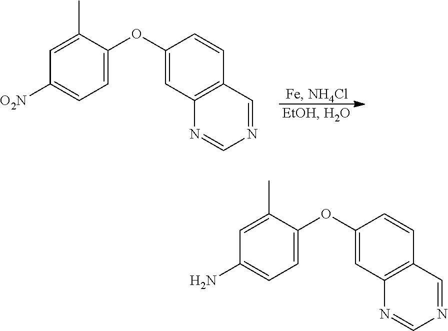 [SMILES: Cc1cc([N+](=O)[O-])ccc1Oc1ccc2cncnc2c1 ; Cc1cc(N)ccc1Oc1ccc2cncnc2c1]

To a solution of 7-(2-methyl-4-nitrophenoxy)quinazoline (400.0 mg, 1.42 mmol) in EtOH (8.0 mL)/H₂O (2.0 mL) were added Fe (49.6 mg, 0.89 mmol) and NH₄Cl (380.4 mg, 7.11 mmol) at room temperature. The resulting mixture was stirred at 80° C. for 3 h. After the reaction was completed, the resulting mixture was filtered. The filtrate was concentrated under reduced pressure. The residue was purified by flash column chromatography with CH₂Cl₂/CH₃OH (10/1, v/v) to afford 3-methyl-4-(quinazolin-7-yloxy)aniline (260.0 mg, 72%) as a yellow solid. LCMS (ESI, m/z): [M+H]⁺=252.1.

### Step 4. Synthesis of 6-iodo-N-[3-methyl-4-(quinazolin-7-yloxy)phenyl]quinazolin-4-amine

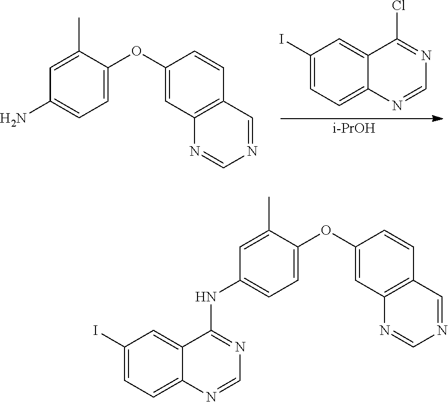 [SMILES: "i-PrOH" ; Cc1cc(N)ccc1Oc1ccc2cncnc2c1 ; Clc1ncnc2ccc(I)cc12 ; Cc1cc(Nc2ncnc3ccc(I)cc23)ccc1Oc1ccc2cncnc2c1]

To a solution of 3-methyl-4-(quinazolin-7-yloxy)aniline (240.0 mg, 0.96 mmol) in i-PrOH (5.0 mL) was added 4-chloro-6-iodoquinazoline (277.4 mg, 0.96 mmol) at room temperature. The resulting mixture was stirred at room temperature for 2 h. After the reaction was completed, the resulting mixture was concentrated under reduced pressure. The residue was purified by flash column chromatography with petroleum ether/ethyl acetate (20/80, v/v) to afford 6-iodo-N-[3-methyl-4-(quinazolin-7-yloxy)phenyl]quinazolin-4-amine (270.0 mg, 55%) as a yellow solid. LCMS (ESI, m/z): [M+H]⁺=506.0.

### Step 5. Synthesis of 1-(4-{[3-methyl-4-(quinazolin-7-yloxy)phenyl]amino}quinazolin-6-yl)-3-methylidenepyrrolidin-2-one (Compound 50)

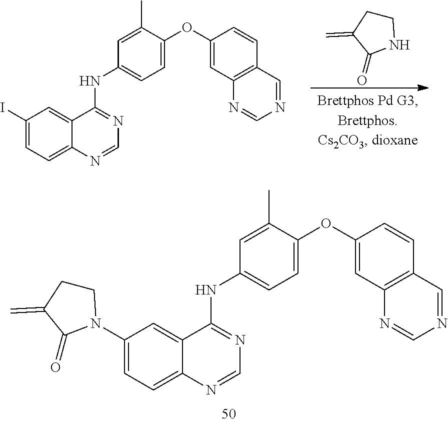

To a solution of 6-iodo-N-[3-methyl-4-(quinazolin-7-yloxy)phenyl]quinazolin-4-amine (100.0 mg, 0.20 mmol) in 1,4-dioxane (3.0 mL) were added 3-methylidenepyrrolidin-2-one (23.1 mg, 0.24 mmol), Cs₂CO₃ (193.4 mg, 0.59 mmol), BrettPhos Pd G3 (18.0 mg, 0.02 mmol) and BrettPhos (21.2 mg, 0.04 mmol) at room temperature under N₂. The mixture was stirred at 100° C. for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) and then purified by Prep-HPLC with the following conditions: (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 35% B to 35% B in 15 min; Wave Length: 254 nm) to afford 1-(4-{[3-methyl-4-(quinazolin-7-yloxy)phenyl]amino}quinazolin-6-yl)-3-methylidenepyrrolidin-2-one (Compound 50) (13.7 mg, 14%) as a write solid. LCMS (ESI, m/z): [M+H]⁺=475.1. ¹H NMR (400 MHz, DMSO-d₆): δ 9.89 (s, 1H), 9.51 (s, 1H), 9.18 (s, 1H), 8.72-8.69 (m, 1H), 8.60 (s, 1H), 8.37 (d, J=2.0 Hz, 1H), 8.23 (d, J=8.8 Hz, 1H), 7.87-7.82 (m, 3H), 7.61-7.58 (m, 1H), 7.27-7.25 (m, 1H), 7.00 (d, J=2.0 Hz, 1H), 6.00 (s, 1H), 5.56 (s, 1H), 4.09-4.06 (m, 2H), 3.01-2.98 (m, 2H), 2.20 (s, 3H).

### Example S48: Synthesis of (4S)-4-methyl-1-[4-({3-methyl-4-[(1-methyl-1,3-benzodiazol-5-yl)methyl]phenyl}amino)quinazolin-6-yl]-3-methylidenepyrrolidin-2-one (Compound 51)

### Step 1. Synthesis of 6-iodo-N-{3-methyl-4-[(1-methyl-1,3-benzodiazol-5-yl)methyl]phenyl}quinazolin-4-amine

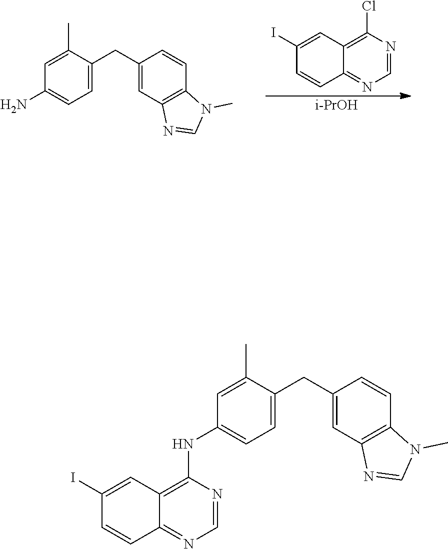 [SMILES: "i-PrOH" ; Cc1cc(N)ccc1Cc1ccc2c(c1)ncn2C ; Clc1ncnc2ccc(I)cc12 ; Cc1cc(Nc2ncnc3ccc(I)cc23)ccc1Cc1ccc2c(c1)ncn2C]

To a mixture of 3-methyl-4-[(1-methyl-1,3-benzodiazol-5-yl)methyl]aniline (180.0 mg, 0.72 mmol) in isopropyl alcohol (10.0 mL) was added 4-chloro-6-iodoquinazoline (208.0 mg, 0.72 mmol) at room temperature. The resulting mixture was stirred at 50° C. for 1 h. After the reaction was completed, the resulting mixture was concentrated under reduced pressure. The residue was purified by flash column chromatography with CH₂Cl₂/MeOH (10/1, v/v) to afford 6-iodo-N-{3-methyl-4-[(1-methyl-1,3-benzodiazol-5-yl)methyl]phenyl}quinazolin-4-amine (240.0 mg, 66%) as a yellow oil. LCMS (ESI, m/z): [M+H]⁺=506.1.

### Step 2. Synthesis of (4S)-4-methyl-1-[4-({3-methyl-4-[(1-methyl-1,3-benzodiazol-5-yl)methyl]phenyl}amino)quinazolin-6-yl]-3-methylidenepyrrolidin-2-one (Compound 51)

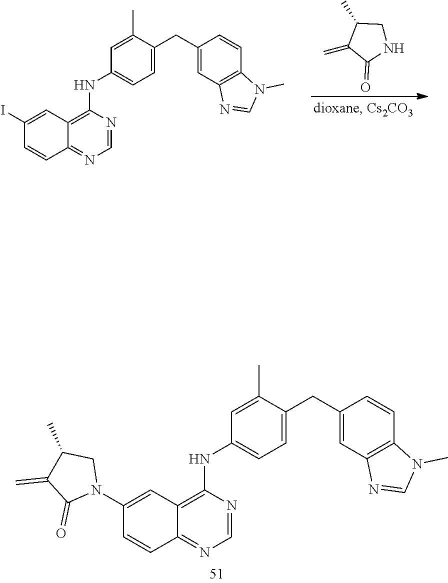

To a stirred mixture of 6-iodo-N-{3-methyl-4-[(1-methyl-1,3-benzodiazol-5-yl)methyl]phenyl}quinazolin-4-amine (100.0 mg, 0.20 mmol) and (4S)-4-methyl-3-methylidenepyrrolidin-2-one (33.0 mg, 0.30 mmol) in dioxane (6.0 mL) were added Cs₂CO₃ (193.4 mg, 0.59 mmol), BrettPhos Pd G3 (35.9 mg, 0.04 mmol) and BrettPhos (42.5 mg, 0.08 mmol) at room temperature under N₂. The resulting mixture was stirred at 100° C. for 16 h. After the reaction was completed, the resulting mixture was concentrated under reduced pressure. The residue was purified by flash column chromatography with CH₂Cl₂/MeOH (5/1, v/v) and then purified by Prep-HPLC with the following conditions (Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 35% B to 45% B in 8 min; Wave Length: 254 nm) to afford (4S)-4-methyl-1-[4-({3-methyl-4-[(1-methyl-1,3-benzodiazol-5-yl)methyl]phenyl}amino)quinazolin-6-yl]-3-methylidenepyrrolidin-2-one (Compound 51) (6.0 mg, 6%) as a white solid. LCMS (ESI, m/z): [M+H]⁺=489.3. ¹H NMR (400 MHz, DMSO-d₆): δ 9.73 (s, 1H), 8.71-8.68 (m, 1H), 8.52 (s, 1H), 8.28 (d, J=2.0 Hz, 1H), 8.12 (s, 1H), 7.81 (d, J=9.2 Hz, 1H), 7.61-7.59 (m, 1H), 7.55 (s, 1H), 7.49 (d, J=8.4 Hz, 1H), 7.40 (s, 1H), 7.20 (d, J=8.0 Hz, 1H), 7.13 (d, J=8.0 Hz, 1H), 6.00 (d, J=2.8 Hz, 1H), 5.55 (d, J=2.4 Hz, 1H), 4.21-4.19 (m, 1H), 4.09 (s, 2H), 3.82 (s, 3H), 3.68-3.52 (m, 1H), 3.21-3.16 (m, 1H), 2.27 (s, 3H), 1.34 (d, J=6.8 Hz, 3H).

### Example S49: Synthesis of 1-[4-({3-methyl-4-[(1-methyl-1,3-benzodiazol-5-yl)methyl]phenyl}amino)quinazolin-6-yl]-3-methylidenepyrrolidin-2-one (Compound 52)

### Synthesis of 1-[4-({3-methyl-4-[(1-methyl-1,3-benzodiazol-5-yl)methyl]phenyl}amino)quinazolin-6-yl]-3-methylidenepyrrolidin-2-one (Compound 52)

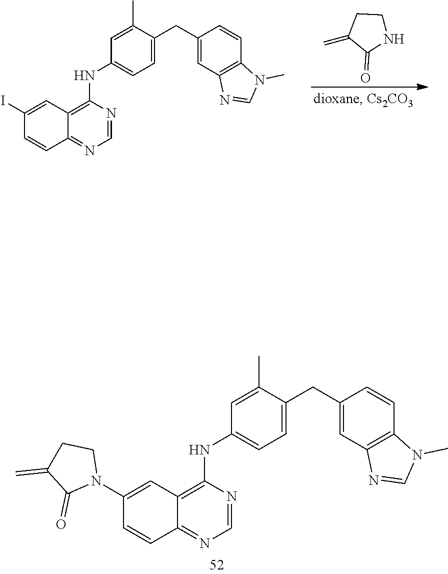

To a mixture of 6-iodo-N-{3-methyl-4-[(1-methyl-1,3-benzodiazol-5-yl)methyl]phenyl}quinazolin-4-amine (80.0 mg, 0.16 mmol) and 3-methylidenepyrrolidin-2-one (23.1 mg, 0.24 mmol) in dioxane (6.0 mL) were added Cs₂CO₃ (154.7 mg, 0.47 mmol), BrettPhos (34.0 mg, 0.06 mmol) and BrettPhos Pd G3 (28.7 mg, 0.03 mmol) at room temperature under N₂. The resulting mixture was stirred at 100° C. for 16 h. After the reaction was completed, the resulting mixture was concentrated under reduced pressure. The residue was purified by flash column chromatography with CH₂Cl₂/MeOH (5/1, v/v) and then purified by Prep-HPLC with the following conditions (Column: XSelect CSH Prep C18 OBD Column, 19×250 mm, 5 μm; Mobile Phase A: water (10 mmol/L NH₄HCO₃), Mobile Phase B: MeOH Preparative; Flow rate: 25 mL/min; Gradient: 60% B to 75% B in 12 min; Wave Length: 254 nm) to afford 1-[4-({3-methyl-4-[(1-methyl-1,3-benzodiazol-5-yl)methyl]phenyl}amino)quinazolin-6-yl]-3-methylidenepyrrolidin-2-one (Compound 52) (7.8 mg, 10%) as a white solid. LCMS (ESI, m/z): [M+H]⁺=475.3. ¹H NMR (400 MHz, DMSO-d₆): δ 9.73 (s, 1H), 8.70-8.67 (m, 1H), 8.52 (s, 1H), 8.32 (d, J=1.6 Hz, 1H), 8.12 (s, 1H), 7.81 (d, J=9.2 Hz, 1H), 7.61-7.55 (m, 2H), 7.48 (d, J=8.4 Hz, 1H), 7.40 (s, 1H), 7.20-7.12 (m, 2H), 5.98 (s, 1H), 5.55 (s, 1H), 4.09-4.04 (m, 4H), 3.82 (s, 3H), 2.99-2.96 (m, 2H), 2.27 (s, 3H).

### Example S50: Synthesis of (3E)-3 [3-(dimethylamino)propylidene]-1-[4-({3-methyl-4-[(1-methyl-1,3-benzodiazol-5 yl)oxy]phenyl}amino)pyrido[3,2-d]pyrimidin-6-yl]pyrrolidin-2-one (Compound 53)

### Synthesis of (3E)-3-[3-(dimethylamino)propylidene]-1-[4-({3-methyl-4-[(1-methyl-1,3-benzodiazol-5-yl)oxy]phenyl}amino)pyrido[3,2-d]pyrimidin-6-yl]pyrrolidin-2-one (Compound 53)

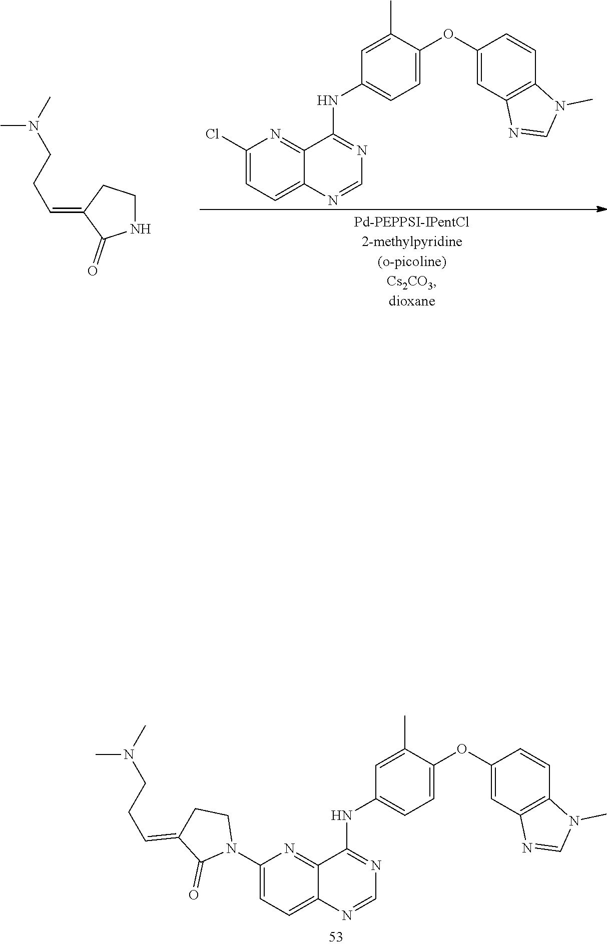

To a solution of (3E)-3-[3-(dimethylamino)propylidene]pyrrolidin-2-one (90.0 mg, 0.54 mmol) in dioxane (10.0 mL) was added 6-chloro-N-{3-methyl-4-[(1-methyl-1,3-benzodiazol-5-yl)oxy]phenyl}pyrido[3,2-d]pyrimidin-4-amine (148.6 mg, 0.36 mmol), Cs₂CO₃ (81.5 mg, 1.07 mmol) and Pd-PEPPSI-IPentCl 2-methylpyridine (o-picoline) (30.0 mg, 0.04 mmol) at 100° C. under N₂. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: (Column: Xselect CSH C18 OBD Column 30×150 mm, 5 μm; Mobile Phase A: Water (0.1% FA), Mobile Phase B: ACN; Flow rate: 60 mL/min; Gradient: 8% B to 20% B in 8 min; Wave Length: 220 nm) to afford (3E)-3-[3-(dimethylamino)propylidene]-1-[4-({3-methyl-4-[(1-methyl-1,3-benzodiazol-5-yl)oxy]phenyl}amino)pyrido[3,2-d]pyrimidin-6-yl]pyrrolidin-2-one (Compound 53) (33.8 mg, 17%) as a yellow solid. LCMS (ESI, m/z): [M+H]⁺=549.4. ¹H NMR (400 MHz, DMSO-d₆): δ 9.52 (s, 1H), 8.97 (d, J=9.2 Hz, 1H), 8.58 (s, 1H), 8.25-8.22 (m, 1H), 8.18 (s, 1H), 7.83 (d, J=2.4 Hz, 1H), 7.79-7.76 (m, 1H), 7.57 (d, J=8.8 Hz, 1H), 7.10 (d, J=2.4 Hz, 1H), 7.02-6.99 (m, 1H), 6.91 (d, J=8.4 Hz, 1H), 6.62-6.59 (m, 1H), 4.35-4.31 (m, 2H), 3.84 (s, 3H), 2.88-2.84 (m, 2H), 2.45-2.33 (m, 4H), 2.27 (s, 3H), 2.18-2.16 (m, 6H).

### Example S51: Synthesis of (3E)-3-[2-(dimethylamino)ethylidene]-1-[4-({3-methyl-4-[(1-methyl-1,3-benzodiazol-5 yl)methyl]phenyl}amino)pyrido[3,2-d]pyrimidin-6-yl]pyrrolidin-2-one (Compound 54)

### Synthesis of (3E)-3-[2-(dimethylamino)ethylidene]-1-[4-({3-methyl-4-[(1-methyl-1,3-benzodiazol-5-yl)methyl]phenyl}amino)pyrido[3,2-d]pyrimidin-6-yl]pyrrolidin-2-one (Compound 54)

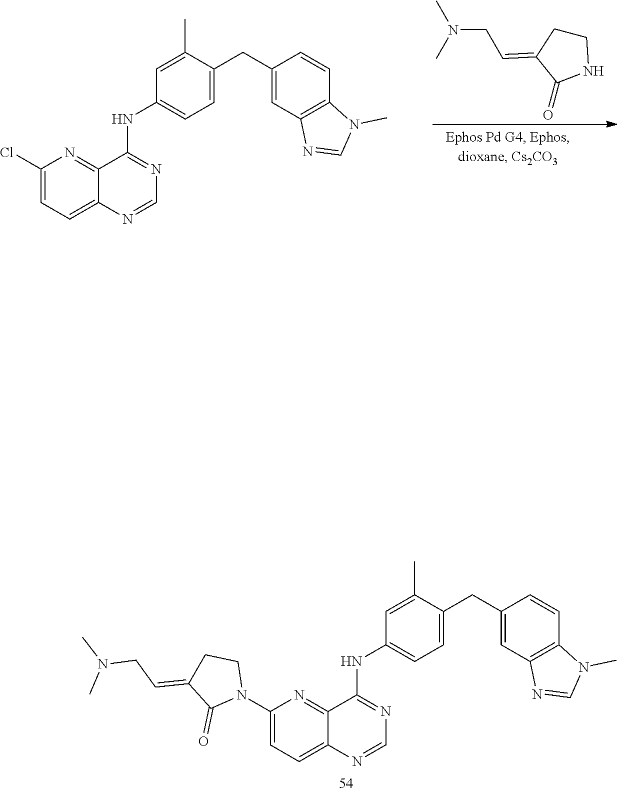

To a solution of 6-chloro-N-{3-methyl-4-[(1-methyl-1,3-benzodiazol-5-yl)methyl]phenyl}pyrido[3,2-d]pyrimidin-4-amine (150.0 mg, 0.36 mmol) in dioxane (10.0 mL) was added (3E)-3-[2-(dimethylamino)ethylidene]pyrrolidin-2-one (55.7 mg, 0.36 mmol), Cs₂CO₃ (294.5 mg, 0.91 mmol), EPhos (38.7 mg, 0.07 mmol) and Ephos Pd G4 (33.2 mg, 0.04 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions Column: (Xbridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 34% B to 44% B in 8 min; Wave Length: 254 nm) to afford (3E)-3-[2-(dimethylamino)ethylidene]-1-[4-({3-methyl-4-[(1-methyl-1,3-benzodiazol-5-yl)methyl]phenyl}amino)pyrido[3,2-d]pyrimidin-6-yl]pyrrolidin-2-one (Compound 54) (13.1 mg, 6%) as a white solid. LCMS (ESI, m/z): [M+H]⁺=533.4. ¹H NMR (400 MHz, DMSO-d₆): δ 9.45 (s, 1H), 8.96 (d, J=9.2 Hz, 1H), 8.58 (s, 1H), 8.24 (d, J=9.2 Hz, 1H), 8.11 (s, 1H), 7.79-7.76 (m, 1H), 7.69 (s, 1H), 7.48 (d, J=8.0 Hz, 1H), 7.38 (s, 1H), 7.23 (d, J=8.4 Hz, 1H), 7.12 (d, J=8.4 Hz, 1H), 6.59-6.54 (m, 1H), 4.35-4.31 (m, 2H), 4.09 (s, 2H), 3.81 (s, 3H), 3.11 (d, J=6.4 Hz, 2H), 2.88-2.84 (m, 2H), 2.26 (s, 3H), 2.21 (s, 6H).

### Example S52: Synthesis of 1-(difluoromethyl)-4-[4-({6-[(3E)-3-[2-(dimethylamino)ethylidene]-2-oxopyrrolidin-1-yl]pyrido[3,2-d]pyrimidin-4 yl}amino)-2-methylphenoxy]pyridin-2-one (Compound 55)

### Step 1. Synthesis of 4-[4-({6-chloropyrido[3,2-d]pyrimidin-4-yl}amino)-2-methylphenoxy]-1-(difluoromethyl)pyridin-2-one

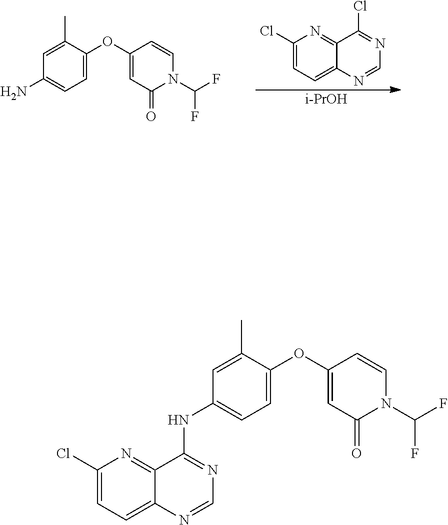 [SMILES: "i-PrOH" ; Cc1cc(N)ccc1Oc1ccn(C(F)F)c(=O)c1 ; Clc1ccc2ncnc(Cl)c2n1 ; Cc1cc(Nc2ncnc3ccc(Cl)nc23)ccc1Oc1ccn(C(F)F)c(=O)c1]

To a solution 4,6-dichloropyrido[3,2-d]pyrimidine (225.3 mg, crude) in i-PrOH (3.0 mL) was added 4-(4-amino-2-methylphenoxy)-1-(difluoromethyl)pyridin-2-one (300.0 mg, 1.13 mmol) at 0° C. under N₂. The resulting mixture was stirred at room temperature for 2 h. After the reaction was completed, the mixture was evaporated in vacuo. The residue was purified by flash column chromatography with CH₂Cl₂/MeOH (5/1, v/v) to afford 4-[4-({6-chloropyrido[3,2-d]pyrimidin-4-yl}amino)-2-methylphenoxy]-1-(difluoromethyl)pyridin-2-one (300.0 mg, 61%) as a yellow solid. LCMS (ESI, m/z): [M+H]⁺=430.1.

### Step 2. Synthesis of 1-(difluoromethyl)-4-[4-({6-[(3E)-3-[2-(dimethylamino)ethylidene]-2-oxopyrrolidin-1-yl]pyrido[3,2-d]pyrimidin-4-yl}amino)-2-methylphenoxy]pyridin-2-one (Compound 55)

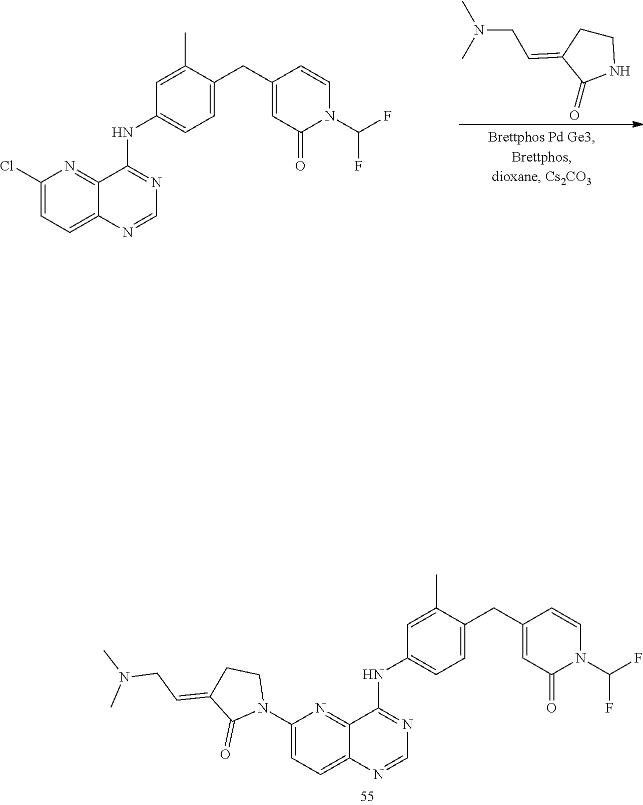 [SMILES: Cc1cc(Nc2ncnc3ccc(Cl)nc23)ccc1Cc1ccn(C(F)F)c(=O)c1 ; CN(C)C/C=C1\CCNC1=O ; Cc1cc(Nc2ncnc3ccc(N4CC/C(=C\CN(C)C)C4=O)nc23)ccc1Cc1ccn(C(F)F)c(=O)c1]

To a solution 4-[4-({6-chloropyrido[3,2-d]pyrimidin-4-yl}amino)-2-methylphenoxy]-1-(difluoromethyl)pyridin-2-one (260.0 mg, 0.61 mmol) in dioxane (10.0 mL) were added (3E)-3-[2-(dimethylamino)ethylidene]pyrrolidin-2-one (111.9 mg, 0.73 mmol), Cs₂CO₃ (492.7 mg, 1.51 mmol), BrettPhos (64.9 mg, 0.12 mmol) and BrettPhos Pd G3 (54.8 mg, 0.06 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions Column: (YMC-Actus Triart C18 ExRS, 30×150 mm, 5 μm; Mobile Phase A: Water (10 mmol/L NH₄HCO₃), Mobile Phase B: ACN; Flow rate: 60 mL/min; Gradient: 42% B to 52% B in 8 min; Wave Length: 254 nm) to afford 1-(difluoromethyl)-4-[4-({6-[(3E)-3-[2-(dimethylamino)ethylidene]-2-oxopyrrolidin-1-yl]pyrido[3,2-d]pyrimidin-4-yl}amino)-2-methylphenoxy]pyridin-2-one (Compound 55) (5.0 mg, 1%) as a white solid. LCMS (ESI, m/z): [M+H]⁺=548.3. ¹H NMR (400 MHz, DMSO-d₆): δ 9.60 (s, 1H), 8.99 (d, J=9.2 Hz, 1H), 8.64 (s, 1H), 8.27 (d, J=9.2 Hz, 1H), 7.99-7.94 (m, 2H), 7.89 (d, J=7.6 Hz, 1H), 7.80-7.64 (m, 1H), 7.24 (d, J=8.4 Hz, 1H), 6.60-6.55 (m, 1H), 6.39-6.3-6 (m, 1H), 5.36 (d, J=2.4 Hz, 1H), 4.37-4.34 (m, 2H), 3.12 (d, J=6.8 Hz, 2H), 2.89-2.85 (m, 2H), 2.21-2.19 (m, 9H).

### Example S53: Synthesis of 1-{7-[2-(dimethylamino)ethoxy]-4-[(3-methyl-4-{[1,2,4]triazolo[1,5-a]pyridin-7-yloxy}phenyl)amino]quinazolin-6-yl}-3-methylidenepyrrolidin-2-one (Compound 56)

### Step 1. Synthesis of 6-bromo-7-[2-(dimethylamino)ethoxy]-N-(3-methyl-4-{[1,2,4]triazolo[1,5-a]pyridin-7-yloxy}phenyl)quinazolin-4-amine

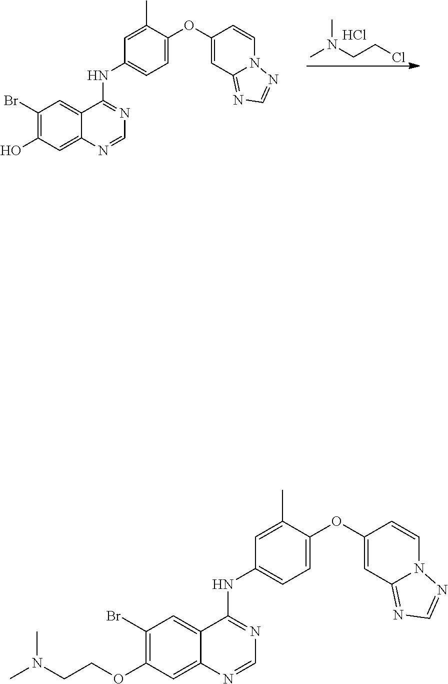

To a solution of 6-bromo-4-[(3-methyl-4-{[1,2,4]triazolo[1,5-a]pyridin-7-yloxy}phenyl)amino]quinazolin-7-ol (500.0 mg, 1.08 mmol) in DMF (15.0 mL) was added (2-chloroethyl)dimethylamine hydrochloride (279.8 mg, 1.94 mmol) and K₂CO₃ (596.6 mg, 4.32 mmol) at room temperature. The resulting solution was stirred at 65° C. for 16 h. After the reaction was completed, the resulting mixture was cooled to room temperature and filtered. The filtrate was purified by reverse phase flash column chromatography with CH₃CN/H₂O (80/20, v/v) to afford 6-bromo-7-[2-(dimethylamino)ethoxy]-N-(3-methyl-4-{[1,2,4]triazolo[1,5-a]pyridin-7-yloxy}phenyl)quinazolin-4-amine (360.0 mg, 62%) as a yellow solid. LCMS (ESI, m/z): [M+H]⁺=534.1.

### Step 2. Synthesis of 1-{7-[2-(dimethylamino)ethoxy]-4-[(3-methyl-4-{[1,2,4]triazolo[1,5-a]pyridin-7-yloxy}phenyl)amino]quinazolin-6-yl}-3-methylidenepyrrolidin-2-one (Compound 56)

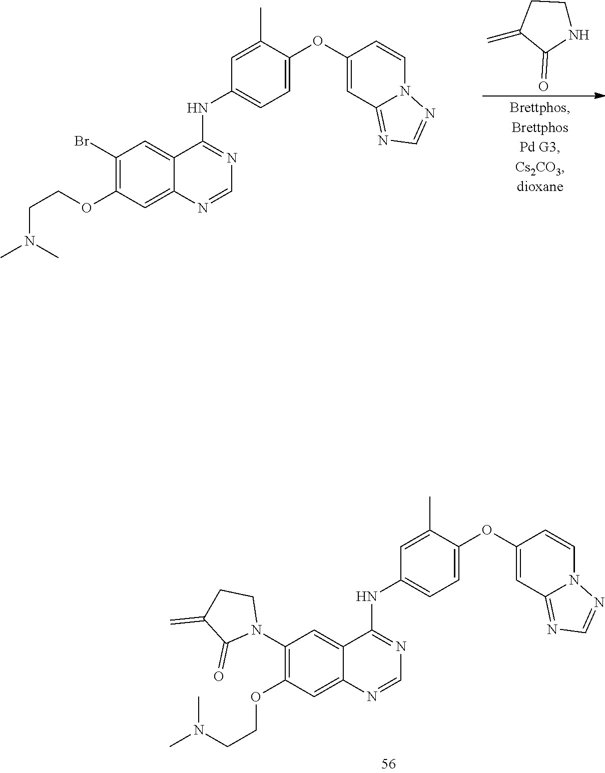

To a solution of 6-bromo-7-[2-(dimethylamino)ethoxy]-N-(3-methyl-4-{[1,2,4]triazolo[1,5-a]pyridin-7-yloxy}phenyl)quinazolin-4-amine (200.0 mg, 0.37 mmol) in dioxane (10.0 mL) was added 3-methylidenepyrrolidin-2-one (109.0 mg, crude), Cs₂CO₃ (731.6 mg, 2.24 mmol), BrettPhos (40.2 mg, 0.08 mmol) and BrettPhos Pd G3 (33.9 mg, 0.04 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep Phenyl OBD Column, 19×250 mm, 5 μm; Mobile Phase A: Water (10 mmol/L NH₄HCO₃), Mobile Phase B: MeOH Preparative; Flow rate: 25 mL/min; Gradient: 60% B to 60% B in 19 min, Wave Length: 254 nm) to afford 1-{7-[2-(dimethylamino)ethoxy]-4-[(3-methyl-4-{[1,2,4]triazolo[1,5-a]pyridin-7-yloxy}phenyl)amino]quinazolin-6-yl}-3-methylidenepyrrolidin-2-one (Compound 56) (3.2 mg, 1%) as a white solid. LCMS (ESI, m/z): [M+H]⁺=511.4. ¹H NMR (400 MHz, DMSO-d₆): δ 9.76 (s, 1H), 8.94 (d, J=7.6 Hz, 1H), 8.59-8.56 (m, 2H), 8.39 (s, 1H), 7.87-7.84 (m, 2H), 7.37 (s, 1H), 7.21 (d, J=8.8 Hz, 1H), 7.05-7.02 (m, 1H), 6.81 (d, J=2.4 Hz, 1H), 5.91 (s, 1H), 5.53 (s, 1H), 4.29-4.26 (m, 2H), 3.87-3.84 (m, 2H), 2.98-2.96 (m, 2H), 2.71-2.68 (m, 2H), 2.23 (s, 6H), 2.06 (s, 3H).

### Example S54: Synthesis of (4S)-4-methyl-1-[4-({3-methyl-4-[(1-methyl-1,3-benzodiazol-5-yl)oxy]phenyl}amino)pyrido[3,2-d]pyrimidin-6-yl]-3-methylidenepyrrolidin-2-one (Compound 59)

### Step 1. Synthesis of (4S)-4-methyl-1-[4-({3-methyl-4-[(1-methyl-1,3-benzodiazol-5-yl)oxy]phenyl}amino)pyrido[3,2-d]pyrimidin-6-yl]-3-methylidenepyrrolidin-2-one (Compound 59)

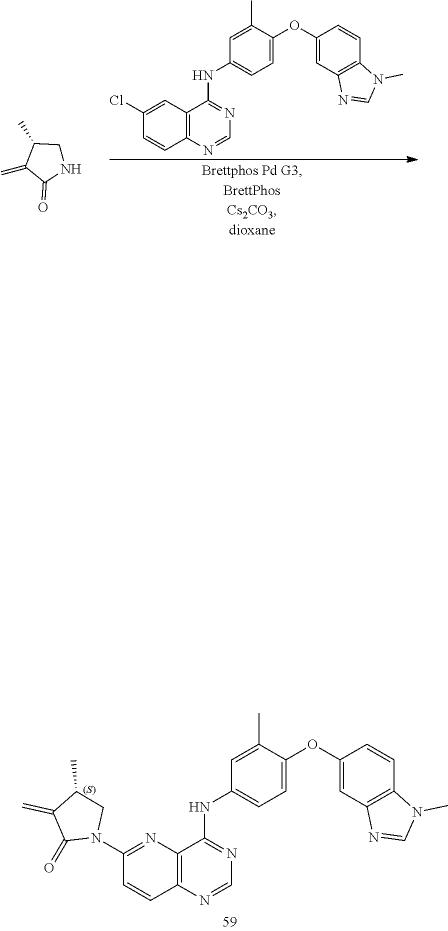

To a solution of 6-chloro-N-{3-methyl-4-[(1-methyl-1,3-benzodiazol-5-yl)oxy]phenyl}pyrido[3,2-d]pyrimidin-4-amine (300.0 mg, 0.70 mmol) in dioxane (12.0 mL) was added (4S)-4-methyl-3-methylidenepyrrolidin-2-one (88.0 mg, 0.80 mmol), BrettPhos (77.3 mg, 0.22 mmol), BrettPhos Pd G3 (65.2 mg, 0.11 mmol) and Cs₂CO₃ (1172.5 mg, 2.16 mmol) at room temperature under N₂. The resulting mixture was stirred at 100° C. for 4 h. After the reaction was completed, the mixture was concentrated under vacuum. The residue was purified by flash column chromatography with CH₂Cl₂/MeOH (90/10, v/v) and then purified by Prep-HPLC with the following conditions: (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 42% B to 52% B in 8 min; Wave Length: 254 nm) to afford (4S)-4-methyl-1-[4-({3-methyl-4-[(1-methyl-1,3-benzodiazol-5-yl)oxy]phenyl}amino)pyrido[3,2-d]pyrimidin-6-yl]-3-methylidenepyrrolidin-2-one (Compound 59) (10.1 mg, 29%) as a yellow solid. LCMS (ESI, m/z): [M+H]⁺=492.2. ¹H NMR (400 MHz, DMSO-d₆): δ 9.54 (s, 1H), 8.95 (d, J=9.2 Hz, 1H), 8.58 (s, 1H), 8.25 (d, J=9.2 Hz, 1H), 8.17 (s, 1H), 7.82 (d, J=2.0 Hz, 1H), 7.77-7.74 (m, 1H), 7.57 (d, J=8.8 Hz, 1H), 7.11 (d, J=2.4 Hz, 1H), 7.02-6.99 (m, 1H), 6.92 (d, J=8.8 Hz, 1H), 6.09 (d, J=2.8 Hz, 1H), 5.63 (d, J=2.4 Hz, 1H), 4.61-4.56 (m, 1H), 3.85-3.81 (m, 4H), 3.18-3.13 (m, 1H), 2.28 (s, 3H), 1.35 (d, J=6.8 Hz, 3H).

### Example S55: Synthesis of (E)-1-(4-(4-([1,2,4]triazolo[1,5-a]pyridin-7-yloxy)-3-methylphenylamino)pyrido[3,4-d]pyrimidin-6-yl)-3-(3-(dimethylamino)propylidene)pyrrolidin-2-one formate (Compound 60)

### Step 1. Synthesis of (E)-1-(4-(4-([1,2,4]triazolo[1,5-a]pyridin-7-yloxy)-3-methylphenylamino)pyrido[3,4-d]pyrimidin-6-yl)-3-(3-(dimethylamino)propylidene)pyrrolidin-2-one formate (Compound 60)

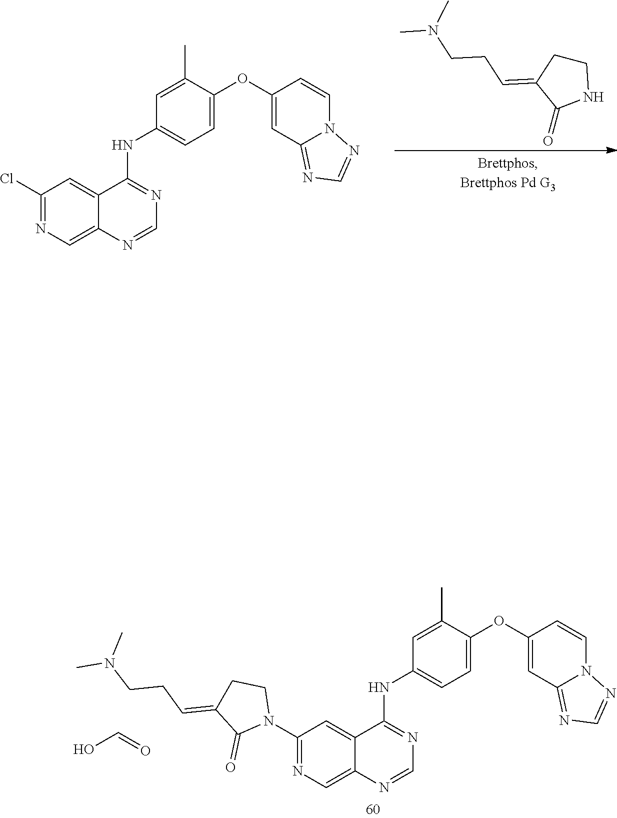

To a solution of 6-chloro-N-(3-methyl-4-{[1,2,4]triazolo[1,5-a]pyridin-7-yloxy}phenyl)pyrido[3,4-d]pyrimidin-4-amine (150.0 mg, 0.37 mmol) in 1,4-dioxane (5.0 mL) was added (3E)-3-[3-(dimethylamino)propylidene]pyrrolidin-2-one (62.5 mg, 0.31 mmol), Cs₂CO₃ (363.1 mg, 1.11 mmol), BrettPhos (39.9 mg, 0.08 mmol) and BrettPhos Pd G3 (33.6 mg, 0.04 mmol) at room temperature under N₂. The resulting mixture was stirred at 80° C. for 16 h under N₂. After the reaction was completed, the reaction mixture was quenched with water and extracted with ethyl acetate. The combined organic layers were washed with brine, dried over anhydrous Na₂SO₄ and filtered. The filtrate was concentrated under reduced pressure. The residue was purified by flash column chromatography with CH₂Cl₂/CH₃OH (75/25, v/v) and then purified by Prep-HPLC with the following conditions: (Column: XBridge Prep Phenyl OBD Column, 19×250 mm, 5 μm; Mobile Phase A: Water (0.05% FA), Mobile Phase B: MeOH-HPLC; Flow rate: 25 mL/min; Gradient: 55% B to 55% B in 10 min; Wave Length: 254 nm) to afford (E)-1-(4-(4-([1,2,4]triazolo[1,5-a]pyridin-7-yloxy)-3-methylphenylamino)pyrido[3,4-d]pyrimidin-6-yl)-3-(3-(dimethylamino)propylidene)pyrrolidin-2-one formate (Compound 60) (10.5 mg, 5%) as a yellow solid. LCMS (ESI, m/z): [M+H]⁺=536.3. ¹H NMR (400 MHz, DMSO-d₆): δ 10.28 (s, 1H), 9.12 (s, 1H), 9.06 (d, J=8.8 Hz, 1H), 8.94 (d, J=7.2 Hz, 1H), 8.63 (s, 1H), 8.39 (s, 1H), 8.28 (s, 1H), 7.85-7.80 (m, 2H), 7.23 (d, J=8.4 Hz, 1H), 7.05-7.03 (m, 1H), 6.83 (d, J=2.4 Hz, 1H), 6.58-6.54 (m, 1H), 4.22-4.14 (m, 2H), 2.89-2.84 (m, 2H), 2.45-2.37 (m, 4H), 2.21 (s, 9H).

### Example S56: Synthesis of (E)-3-(2-(dimethylamino)ethylidene)-1-(4-(3-methyl-4-(quinoxalin-6-yloxy)phenylamino)pyrido[3,2-d]pyrimidin-6-yl)pyrrolidin-2-one (Compound 61)

### Step 1. Synthesis of (E)-3-(2-(dimethylamino)ethylidene)-1-(4-(3-methyl-4-(quinoxalin-6-yloxy)phenylamino)pyrido[3,2-d]pyrimidin-6-yl)pyrrolidin-2-one (Compound 61)

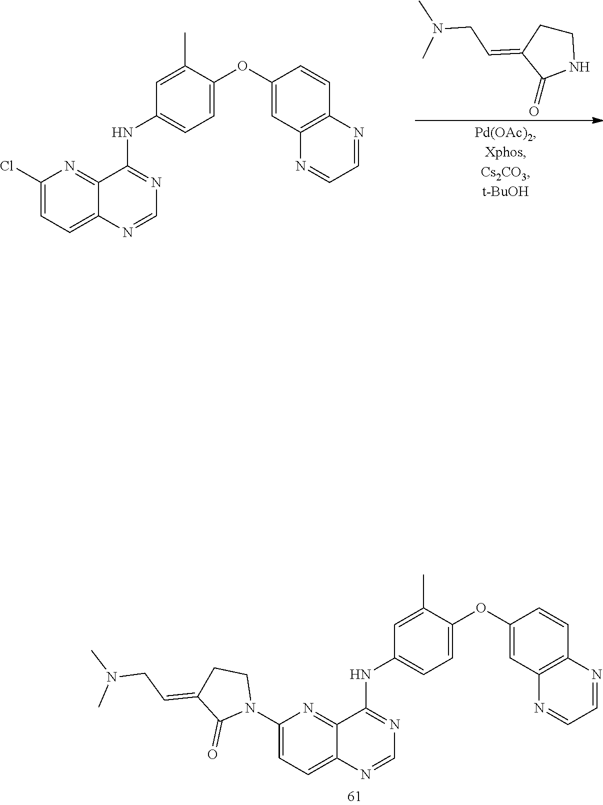 [SMILES: Cc1cc(Nc2ncnc3ccc(Cl)nc23)ccc1Oc1ccc2nccnc2c1 ; CN(C)C/C=C1\CCNC1=O ; Cc1cc(Nc2ncnc3ccc(N4CC/C(=C\CN(C)C)C4=O)nc23)ccc1Oc1ccc2nccnc2c1]

To a solution of 6-chloro-N-(3-methyl-4-(quinoxalin-6-yloxy)phenyl)pyrido[3,2-d]pyrimidin-4-amine (100.0 mg, 0.24 mmol) in t-BuOH (2.0 mL) was added (E)-3-(2-(dimethylamino)ethylidene)pyrrolidin-2-one (55.8 mg, 0.36 mmol), XPhos (23.0 mg, 0.05 mmol), Cs₂CO₃ (157.5 mg, 0.72 mmol) and Pd(OAc)₂ (5.4 mg, 0.02 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) and then purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 45% to 55% in 8 min; 254 nm) to afford (E)-3-(2-(dimethylamino)ethylidene)-1-(4-((3-methyl-4-(quinoxalin-6-yloxy)phenyl)amino)pyrido[3,2-d]pyrimidin-6-yl)pyrrolidin-2-one (Compound 61) (5.8 mg, 4%) as a yellow solid. LCMS (ESI, m/z): [M+H]⁺=533.3. ¹H NMR (400 MHz, DMSO-d₆): δ 9.59 (s, 1H), 8.98 (d, J=9.2 Hz, 1H), 8.87-8.84 (m, 2H), 8.63 (s, 1H), 8.26 (d, J=9.2 Hz, 1H), 8.15 (d, J=9.2 Hz, 1H), 7.98-7.96 (m, 2H), 7.71-7.68 (m, 1H), 7.25 (d, J=8.0 Hz, 1H), 7.14 (d, J=2.8 Hz, 1H), 6.59-6.56 (m, 1H), 4.37-4.33 (m, 2H), 3.11 (d, J=6.8 Hz, 2H), 2.88-2.85 (m, 2H), 2.23-2.21 (m, 9H).

### Example S57: Synthesis of (3E)-3 [2-(dimethylamino)ethylidene]-1-(4-{[3-methyl-4-(pyridin-3-yloxy)phenyl]amino}pyrido[3,2-d]pyrimidin-6-yl)pyrrolidin-2-one (Compound 62)

### Step 1. Synthesis of 3-(2-methyl-4-nitrophenoxy)pyridine

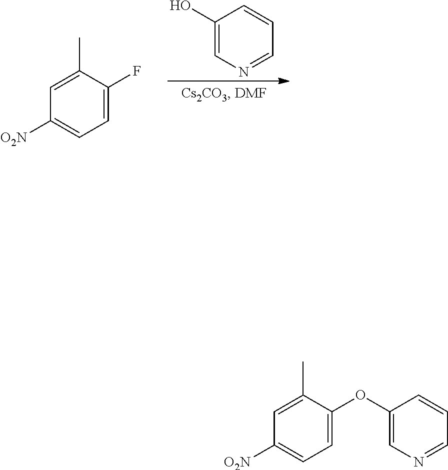 [SMILES: Cc1cc([N+](=O)[O-])ccc1F ; Oc1cccnc1 ; Cc1cc([N+](=O)[O-])ccc1Oc1cccnc1]

To a solution of 1-fluoro-2-methyl-4-nitrobenzene (1.0 g, 6.45 mmol) in DMF (15.0 mL) was added 3-hydroxypyridine (613.1 mg, 6.45 mmol) and Cs₂CO₃ (4.2 g, 12.89 mmol) at room temperature under N₂. The mixture was stirred at 50° C. for 16 h. After the reaction was completed, the resulting mixture was diluted with water and extracted with ethyl acetate. The combined organic layers were washed with brine, dried over anhydrous sodium sulfate and filtered. The filtrate was concentrated under reduced pressure to afford 3-(2-methyl-4-nitrophenoxy)pyridine (1.6 g, crude) as a brown oil. LCMS (ESI, m/z): [M+H]⁺=231.1.

### Step 2. Synthesis of 3-methyl-4-(pyridin-3-yloxy)aniline

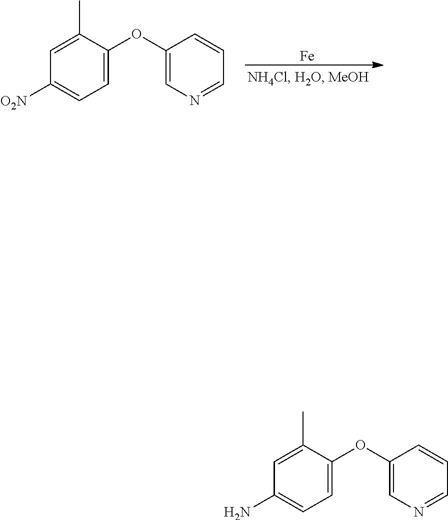

To a solution of 3-(2-methyl-4-nitrophenoxy)pyridine (1.5 g, 6.78 mmol) in MeOH (20.0 mL)/H₂O (4.0 mL) was added NH₄Cl (1.8 g, 33.88 mmol) and Fe (1.9 g, 33.88 mmol) at room temperature. The mixture was stirred at 80° C. for 2 h. After the reaction was completed, the resulting mixture was filtered. The filtrate was concentrated under reduced pressure. The residue was purified by flash column chromatography with CH₂Cl₂/MeOH (10/1, v/v) to afford 3-methyl-4-(pyridin-3-yloxy)aniline (890.0 mg, 65%) as a brown oil. LCMS (ESI, m/z): [M+H]⁺=201.1.

### Step 3. Synthesis of 6-chloro-N-[3-methyl-4-(pyridin-3-yloxy)phenyl]pyrido[3,2-d]pyrimidin-4-amine

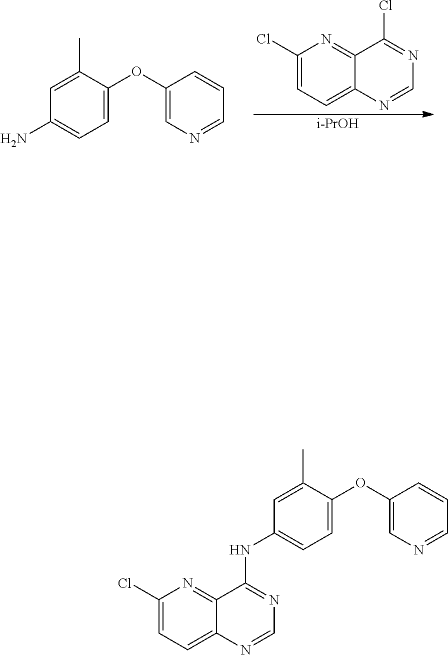 [SMILES: "i-PrOH" ; Cc1cc(N)ccc1Oc1cccnc1 ; Clc1ccc2ncnc(Cl)c2n1 ; Cc1cc(Nc2ncnc3ccc(Cl)nc23)ccc1Oc1cccnc1]

To a solution of 3-methyl-4-(pyridin-3-yloxy)aniline (200.0 mg, 0.99 mmol) in i-PrOH (6.0 mL) was added 4,6-dichloropyrido[3,2-d]pyrimidine (199.8 mg, 0.99 mmol) at 0° C. The resulting mixture was stirred at room temperature for 2 h. After the reaction was completed, the mixture was concentrated under reduced pressure. The residue was purified by flash column chromatography with CH₂Cl₂/MeOH (10/1, v/v) to afford 6-chloro-N-[3-methyl-4-(pyridin-3-yloxy)phenyl]pyrido[3,2-d]pyrimidin-4-amine (440.0 mg, 96%) as a yellow solid. LCMS (ESI, m/z): [M+H]⁺=364.1.

### Step 4. Synthesis of (3E)-3-[2-(dimethylamino)ethylidene]-1-(4-{[3-methyl-4-(pyridin-3-yloxy)phenyl]amino}pyrido[3,2-d]pyrimidin-6-yl)pyrrolidin-2-one (Compound 62)

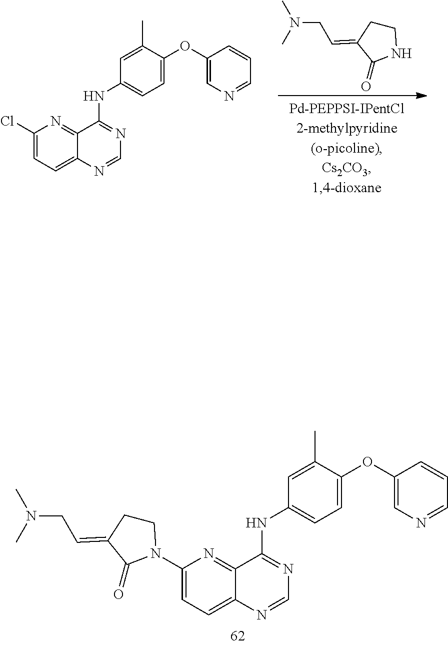

To a solution of 6-chloro-N-[3-methyl-4-(pyridin-3-yloxy)phenyl]pyrido[3,2-d]pyrimidin-4-amine (100.0 mg, 0.28 mmol) in 1,4-dioxane (5.0 mL) was added (3E)-3-[2-(dimethylamino)ethylidene]pyrrolidin-2-one (63.6 mg, 0.41 mmol), Cs₂CO₃ (268.7 mg, 0.83 mmol) and Pd-PEPPSI-IPentCl 2-methylpyridine (o-picoline) (11.6 mg, 0.05 mmol) at room temperature under N₂. The resulting mixture was stirred at 100° C. for 2 h under N₂. After the reaction was completed, the mixture was filtered. The filtrate was concentrated under reduced pressure. The residue was purified by flash column chromatography with CH₂Cl₂/CH₃OH (10/1, v/v) and then purified by Prep-HPLC with the following conditions: (Column: XSelect CSH Prep C18 OBD Column, 19×250 mm, 5 μm; Mobile Phase A: Water (10 mmol/L NH₄HCO₃), Mobile Phase B: MeOH-HPLC; Flow rate: 25 mL/min; Gradient: 75% B to 75% B in 10 min; Wave Length: 254 nm) to afford (3E)-3-[2-(dimethylamino)ethylidene]-1-(4-{[3-methyl-4-(pyridin-3-yloxy)phenyl]amino}pyrido[3,2-d]pyrimidin-6-yl)pyrrolidin-2-one (Compound 62) (24.0 mg, 18%) as a yellow solid. LCMS (ESI, m/z): [M+H]⁺=482.4. ¹H NMR (400 MHz, DMSO-d₆): δ 9.54 (s, 1H), 8.98 (d, J=9.2 Hz, 1H), 8.61 (s, 1H), 8.34-8.24 (m, 3H), 7.89 (d, J=6.4 Hz, 2H), 7.42-7.39 (m, 1H), 7.29 (d, J=8.4 Hz, 1H), 7.08 (d, J=9.2 Hz, 1H), 6.59-6.55 (m, 1H), 4.36-4.32 (m, 2H), 3.11 (d, J=6.4 Hz, 2H), 2.87-2.82 (m, 2H), 2.22-2.08 (m, 9H).

### Example S58: Synthesis of (3E,4R)-3 [2-(dimethylamino)ethylidene]-4-methyl-1-[4-({3-methyl-4-[(1-methyl-1,3-benzodiazol-5 yl)oxy]phenyl}amino)pyrido[3,2-d]pyrimidin-6-yl]pyrrolidin-2-one hydrochloride and (3E,4S)-3-[2-(dimethylamino)ethylidene]-4-methyl-1-[4-({3-methyl-4-[(1-methyl-1,3-benzodiazol-5 yl)oxy]phenyl}amino)pyrido[3,2-d]pyrimidin-6-yl]pyrrolidin-2-one hydrochloride (Compounds 63 and 64)

### Step 1. Synthesis of tert-butyl 3-{2-[(tert-butoxycarbonyl)(methyl)amino]-1-hydroxyethyl}-4-methyl-2-oxopyrrolidine-1-carboxylate

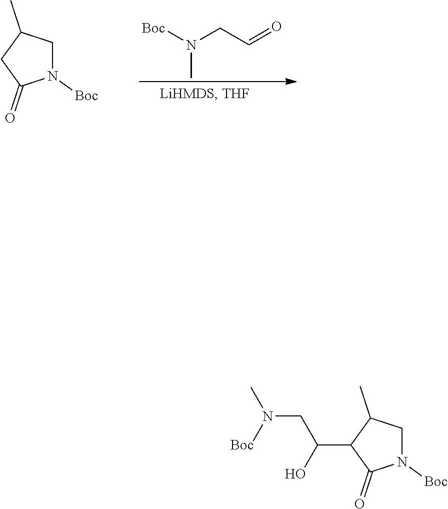 [SMILES: CC1CC(=O)N(C(=O)OC(C)(C)C)C1 ; CN(CC=O)C(=O)OC(C)(C)C ; CC1CN(C(=O)OC(C)(C)C)C(=O)C1C(O)CN(C)C(=O)OC(C)(C)C]

To a solution of tert-butyl 4-methyl-2-oxopyrrolidine-1-carboxylate (8.5 g, 42.7 mmol) in THF (100.0 mL) was added LiHMDS (85.0 mL, 1 mol/L) at −78° C. under N₂. The mixture was stirred at −78° C. for 2 h under N₂. Then a solution of tert-butyl N-methyl-N-(2-oxoethyl)carbamate (7.4 g, 42.7 mmol) in THF (50.0 mL) was added dropwise to the mixture at 78° C. The mixture was stirred at −78° C. for additional 2 h. After the reaction was completed, the reaction mixture was quenched with NH₄Cl (sat.) and extracted with ethyl acetate. The combined organic layer was washed with brine, dried over anhydrous sodium sulfate and filtered. The filtrate was concentrated under vacuum to afford tert-butyl 3-{2-[(tert-butoxycarbonyl)(methyl)amino]-1-hydroxyethyl}-4-methyl-2-oxopyrrolidine-1-carboxylate (16.7 g, crude) as a light yellow oil. LCMS (ESI, m/z): [M+H]⁺=373.2.

### Step 2. Synthesis of tert-butyl 3-{2-[(tert-butoxycarbonyl)(methyl)amino]-1-(methanesulfonyloxy)ethyl}-4-methyl-2-oxopyrrolidine-1-carboxylate

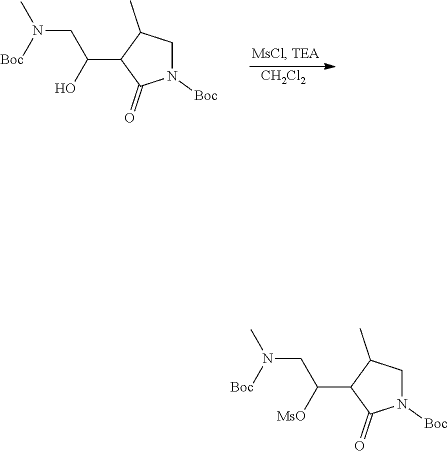 [SMILES: CC1CN(C(=O)OC(C)(C)C)C(=O)C1C(O)CN(C)C(=O)OC(C)(C)C ; "CH2Cl2" ; CC1CN(C(=O)OC(C)(C)C)C(=O)C1C(CN(C)C(=O)OC(C)(C)C)OS(C)(=O)=O]

To a mixture of tert-butyl 3-{2-[(tert-butoxycarbonyl)(methyl)amino]-1-hydroxyethyl}-4-methyl-2-oxopyrrolidine-1-carboxylate (16.0 g, crude) in CH₂Cl₂ (160.0 mL) was added TEA (8.7 g, 85.91 mmol) at room temperature. Then MsCl (9.8 g, 85.91 mmol) was added dropwise to the mixture at 0° C. under N₂. The mixture was stirred at room temperature for 16 h. After the reaction was completed, the mixture was diluted with H₂O and extracted with CH₂Cl₂. The combined organic layer was washed with brine, dried over anhydrous sodium sulfate and filtered. The filtrate was concentrated under vacuum to afford tert-butyl 3-{2-[(tert-butoxycarbonyl)(methyl)amino]-1-(methanesulfonyloxy)ethyl}-4-methyl-2-oxopyrrolidine-1-carboxylate (15.0 g, crude) as a brown oil. LCMS (ESI, m/z): [M+H]⁺=451.2.

### Step 3. Synthesis of tert-butyl (3E)-3-{2-[(tert-butoxycarbonyl)(methyl)amino]ethylidene}-4-methyl-2-oxopyrrolidine-1-carboxylate

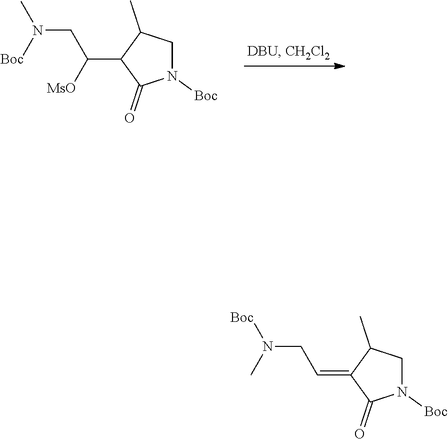 [SMILES: CC1CN(C(=O)OC(C)(C)C)C(=O)C1C(CN(C)C(=O)OC(C)(C)C)OS(C)(=O)=O ; CC1CN(C(=O)OC(C)(C)C)C(=O)/C1=C/CN(C)C(=O)OC(C)(C)C]

To a solution of tert-butyl 3-{2-[(tert-butoxycarbonyl)(methyl)amino]-1-(methanesulfonyloxy)ethyl}-4-methyl-2-oxopyrrolidine-1-carboxylate (15.0 g, crude) in CH₂Cl₂ (200.0 mL) was added DBU (10.1 g, 66.59 mmol) at 0° C. The mixture was stirred at room temperature for 2 h. After the reaction was completed, the mixture was diluted with H₂O and extracted with CH₂Cl₂. The combined organic layer was washed with brine, dried over anhydrous sodium sulfate and filtered. The filtrate was evaporated in vacuo. The residue was purified by flash column chromatography with petroleum ether/EtOAc (2/1, v/v) to afford tert-butyl (3E)-3-{2-[(tert-butoxycarbonyl)(methyl)amino]ethylidene}-4-methyl-2-oxopyrrolidine-1-carboxylate (1.2 g, 10%) as a colorless oil. LCMS (ESI, m/z): [M+H]⁺=355.2.

### Step 4. Synthesis of (E)-4-methyl-3-(2-(methylamino)ethylidene)pyrrolidin-2-one 2,2,2-trifluoroacetate

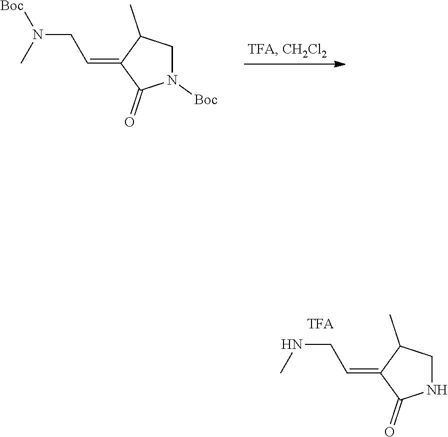 [SMILES: CC1CN(C(=O)OC(C)(C)C)C(=O)/C1=C/CN(C)C(=O)OC(C)(C)C ; CNC/C=C1/C(=O)NCC1C]

To a mixture of tert-butyl (3E)-3-{2-[(tert-butoxycarbonyl)(methyl)amino]ethylidene}-4-methyl-2-oxopyrrolidine-1-carboxylate (1.2 g, 3.4 mmol) in CH₂Cl₂ (12.0 mL) was added TFA (6.0 mL) at room temperature. The mixture was stirred at room temperature for 1 h. After the reaction was completed, the mixture was concentrated under reduced pressure to afford (E)-4-methyl-3-(2-(methylamino)ethylidene)pyrrolidin-2-one 2,2,2-trifluoroacetate (1.0 g, crude) as a brown oil. LCMS (ESI, m/z): [M+H]⁺=155.1.

### Step 5. Synthesis of (3E)-3-[2-(dimethylamino)ethylidene]-4-methylpyrrolidin-2-one

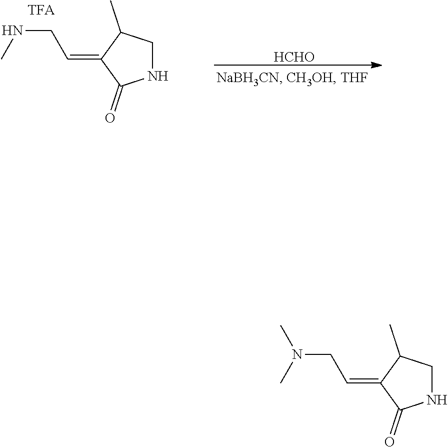

To a mixture of (E)-4-methyl-3-(2-(methylamino)ethylidene)pyrrolidin-2-one 2,2,2-trifluoroacetate (1.0 g, crude) in MeOH (2.0 mL)/THF (10.0 mL) was added HCHO (1.5 g, 37%) at room temperature. The mixture was stirred at room temperature for 1 h. Then NaBH₃CN (2.9 g, 45.4 mmol) was added to the mixture at 0° C. The mixture was stirred at room temperature for 2 h. After the reaction was completed, the resulting mixture was concentrated under reduced pressure. The residue was purified by reverse phase flash chromatography with H₂O/ACN (15/85, v/v) afford (3E)-3-[2-(dimethylamino)ethylidene]-4-methylpyrrolidin-2-one (220.0 mg, 20%) as a yellow solid. LCMS (ESI, m/z): [M+H]⁺=169.1.

### Step 6. Synthesis of (3E)-3-[2-(dimethylamino)ethylidene]-4-methyl-1-[4-({3-methyl-4-[(1-methyl-1,3-benzodiazol-5-yl)oxy]phenyl}amino)pyrido[3,2-d]pyrimidin-6-yl]pyrrolidin-2-one

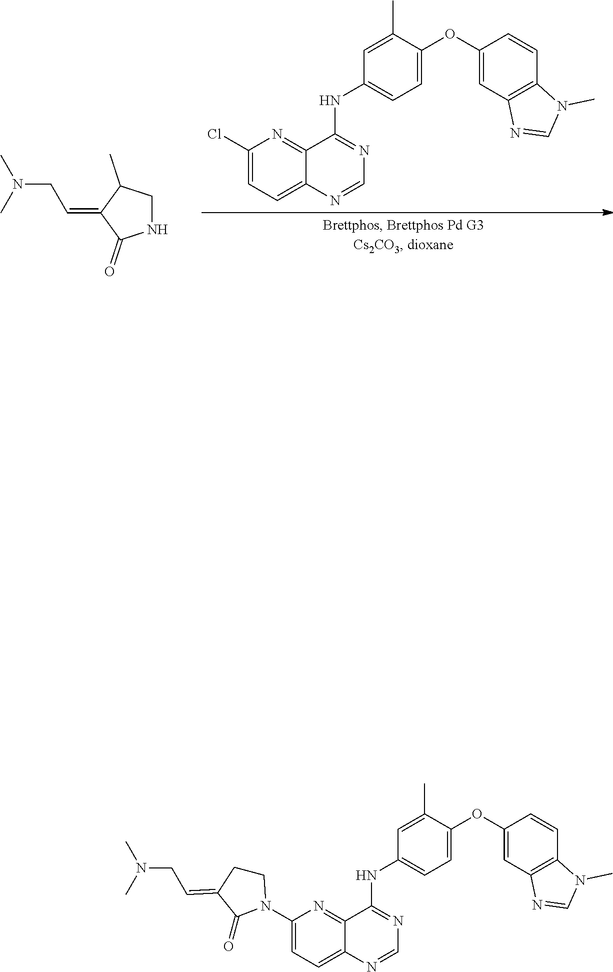

To a solution of (3E)-3-[2-(dimethylamino)ethylidene]-4-methylpyrrolidin-2-one (100.0 mg, 0.59 mmol) in dioxane (10.0 mL) was added 6-chloro-N-{3-methyl-4-[(1-methyl-1,3-benzodiazol-5-yl)oxy]phenyl}pyrido[3,2-d]pyrimidin-4-amine (247.8 mg, 0.59 mmol), Cs₂CO₃ (393.6 mg, 1.18 mmol), Brettphos (63.4 mg, 0.12 mmol) and Brettphos Pd G3 (53.5 mg, 0.06 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with MeOH/H₂O (1/5, v/v) to afford (3E)-3-[2-(dimethylamino)ethylidene]-4-methyl-1-[4-({3-methyl-4-[(1-methyl-1,3-benzodiazol-5-yl)oxy]phenyl}amino)pyrido[3,2-d]pyrimidin-6-yl]pyrrolidin-2-one (60.0 mg, 18%) as a white solid. LCMS (ESI, m/z): [M+H]⁺=549.3.

### Step 7. Separation of (3E,4R)-3-[2-(dimethylamino)ethylidene]-4-methyl-1-[4-({3-methyl-4-[(1-methyl-1,3-benzodiazol-5-yl)oxy]phenyl}amino)pyrido[3,2-d]pyrimidin-6-yl]pyrrolidin-2-one hydrochloride and (3E,4S)-3-[2-(dimethylamino)ethylidene]-4-methyl-1-[4-({3-methyl-4-[(1-methyl-1,3-benzodiazol-5-yl)oxy]phenyl}amino)pyrido[3,2-d]pyrimidin-6-yl]pyrrolidin-2-one hydrochloride (Compounds 63 and 64)

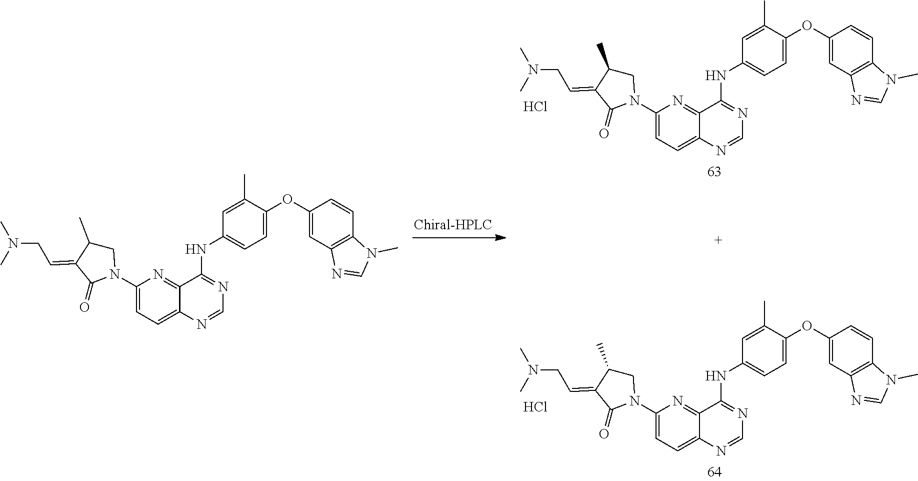

The racemic mixture of (3E)-3-[2-(dimethylamino)ethylidene]-4-methyl-1-[4-({3-methyl-4-[(1-methyl-1,3-benzodiazol-5-yl)oxy]phenyl}amino)pyrido[3,2-d]pyrimidin-6-yl]pyrrolidin-2-one (60.0 mg, 0.11 mmol) was separated by chiral-HPLC with the following conditions: (Column: CHIRAL ART Amylose-SA, 2×25 cm, 5 μm; Mobile Phase A: Hex (0.5% 2M NH₃-MeOH)-HPLC, Mobile Phase B: MeOH: EtOH=1: 1-HPLC; Flow rate: 20 mL/min; Gradient: 50% B to 50% B in 19 min; Wave Length: 220/254 nm; RT1(min): 10.40; RT2(min): 15.61; Sample Solvent: MeOH: DCM=1: 1-HPLC; Injection Volume: 1.5 mL; Number Of Runs: 2) to afford (3E,4R)-3-[2-(dimethylamino)ethylidene]-4-methyl-1-[4-({3-methyl-4-[(1-methyl-1,3-benzodiazol-5-yl)oxy]phenyl}amino)pyrido[3,2-d]pyrimidin-6-yl]pyrrolidin-2-one (assumed) as an off-white solid and (3E,4S)-3-[2-(dimethylamino)ethylidene]-4-methyl-1-[4-({3-methyl-4-[(1-methyl-1,3-benzodiazol-5-yl)oxy]phenyl}amino)pyrido[3,2-d]pyrimidin-6-yl]pyrrolidin-2-one (assumed) as an off-white solid. The free base of each compound was dissolve in Water (0.05% HCl), and then lyophilized to afford (3E)-3-[2-(dimethylamino)ethylidene]-4-methyl-1-[4-({3-methyl-4-[(1-methyl-1,3-benzodiazol-5-yl)oxy]phenyl}amino)pyrido[3,2-d]pyrimidin-6-yl]pyrrolidin-2-one hydrochloride Enantiomer 1 (Retention time: 10.40 min, 24.3 mg, 75%) as a white solid and (3E)-3-[2-(dimethylamino)ethylidene]-4-methyl-1-[4-({3-methyl-4-[(1-methyl-1,3-benzodiazol-5-yl)oxy]phenyl}amino)pyrido[3,2-d]pyrimidin-6-yl]pyrrolidin-2-one hydrochloride Enantiomer 2 (Retention time: 15.61 min, 16.7 mg, 52%) as a white solid. The absolute stereochemistry of Enantiomers 1 and 2 was not assigned. The two enantiomeric structures that could be obtained from chiral separation of the enantiomeric mixture as described above are shown as Compounds 63 and 64 in Table 1.

(3E)-3-[2-(dimethylamino)ethylidene]-4-methyl-1-[4-({3-methyl-4-[(1-methyl-1,3-benzodiazol-5-yl)oxy]phenyl}amino)pyrido[3,2-d]pyrimidin-6-yl]pyrrolidin-2-one hydrochloride Enantiomer 1: RT1(min): 10.40; LCMS (ESI, m/z): [M+H]⁺=549.3. ¹H NMR (400 MHz, DMSO-d₆): δ 10.62 (s, 1H), 9.55 (s, 1H), 8.97 (d, J=9.2 Hz, 1H), 8.59 (s, 1H), 8.28 (d, J=9.2 Hz, 1H), 8.18 (s, 1H), 7.82 (s, 1H), 7.77-7.74 (m, 1H), 7.57 (d, J=8.8 Hz, 1H), 7.09 (d, J=2.0 Hz, 1H), 7.02-7.00 (m, 1H), 6.92 (d, J=8.8 Hz, 1H), 6.61-6.58 (m, 1H), 4.37-4.32 (m, 1H), 4.18-4.15 (m, 1H), 3.99-3.93 (m, 2H), 3.84 (s, 3H), 2.76 (s, 6H), 2.27 (s, 3H), 1.27 (d, J=7.2 Hz, 3H).

(3E)-3-[2-(dimethylamino)ethylidene]-4-methyl-1-[4-({3-methyl-4-[(1-methyl-1,3-benzodiazol-5-yl)oxy]phenyl}amino)pyrido[3,2-d]pyrimidin-6-yl]pyrrolidin-2-one hydrochloride Enantiomer 2: RT2(min): 15.61; LCMS (ESI, m/z): [M+H]⁺=549.3. ¹H NMR (400 MHz, DMSO-d₆): δ 9.54 (s, 1H), 8.97 (d, J=9.2 Hz, 1H), 8.59 (s, 1H), 8.27 (d, J=9.2 Hz, 1H), 8.17 (s, 1H), 7.82 (d, J=2.4 Hz, 1H), 7.77-7.74 (m, 1H), 7.57 (d, J=8.8 Hz, 1H), 7.09 (d, J=2.0 Hz, 1H), 7.02-6.99 (m, 1H), 6.92 (d, J=8.8 Hz, 1H), 6.60-6.57 (m, 1H), 4.36-4.31 (m, 1H), 4.17-4.14 (m, 1H), 3.84-3.76 (m, 6H), 2.64 (s, 6H), 2.27 (s, 3H), 1.28 (d, J=7.2 Hz, 3H).

### Example S59: Synthesis of (3E)-3-[2-(dimethylamino)ethylidene]-1-[4-({4-[(1-ethyl-1,3-benzodiazol-5 yl)oxy]-3-methylphenyl}amino)pyrido[3,2-d]pyrimidin-6-yl]pyrrolidin-2-one (Compound 65)

### Step 1. Synthesis of 4-bromo-N-ethyl-2-nitroaniline

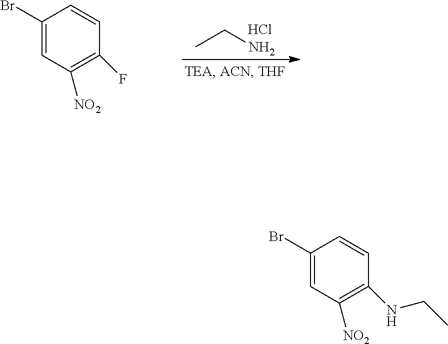

To a solution of 4-bromo-1-fluoro-2-nitrobenzene (5.0 g, 22.73 mmol) in ACN/THF (50.0 mL/50.0 mL) was added TEA (13.8 g, 136.37 mmol) and ethanamine hydrochloride (9.3 g, 113.64 mmol) at room temperature. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford 4-bromo-N-ethyl-2-nitroaniline (5.5 g, crude) as an orange solid. LCMS (ESI, m/z): [M+H]⁺=245.0.

### Step 2. Synthesis of 5-bromo-1-ethyl-1,3-benzodiazole

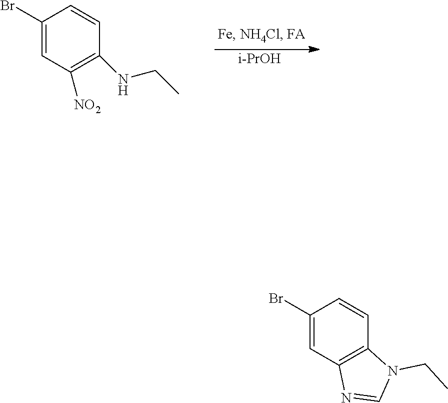

To a solution of 4-bromo-N-ethyl-2-nitroaniline (5.5 g, 22.44 mmol) in isopropanol/formic acid (50.0 mL/50.0 mL) was added NH₄Cl (7.2 g, 134.65 mmol) and Fe (15.9 g, 285.63 mmol) at room temperature. The resulting mixture was stirred at 80° C. for 16 h. After the reaction was completed, the resulting mixture was filtered. The filtrate was concentrated under reduced pressure. The residue was purified by flash column chromatography with CH₂Cl₂/methanol (9/1, v/v) to afford 5-bromo-1-ethyl-1,3-benzodiazole (5.0 g, 98%) as a green oil. LCMS (ESI, m/z): [M+H]⁺=225.0.

### Step 3. Synthesis of 1-ethyl-1,3-benzodiazol-5-ol

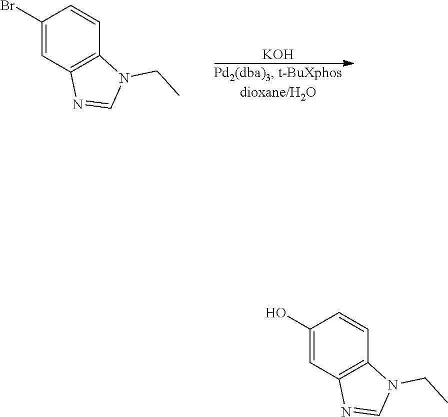

To a solution of 5-bromo-1-ethyl-1,3-benzodiazole (3.4 g, 15.11 mmol) in dioxane/H₂O (20.0 mL/4.0 mL) was added KOH (2.5 g, 45.32 mmol), t-BuXphos (1.3 g, 3.02 mmol) and Pd₂(dba)₃ (1.3 g, 1.51 mmol) at room temperature under N₂. The resulting mixture was stirred at 100° C. for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with DCM/MeOH (93/7, v/v) to afford 1-ethyl-1,3-benzodiazol-5-ol (492.0 mg, 20%) as a brown oil. LCMS (ESI, m/z): [M+H]⁺=163.1.

### Step 4. Synthesis of 1-ethyl-5-(2-methyl-4-nitrophenoxy)-1,3-benzodiazole

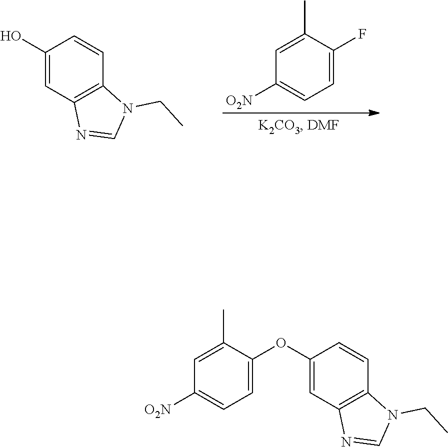 [SMILES: CCn1cnc2cc(O)ccc21 ; Cc1cc([N+](=O)[O-])ccc1F ; CCn1cnc2cc(Oc3ccc([N+](=O)[O-])cc3C)ccc21]

To a solution of 1-ethyl-1,3-benzodiazol-5-ol (492.0 mg, 3.03 mmol) in DMF (10.0 mL) was added 1-fluoro-2-methyl-4-nitrobenzene (564.7 mg, 3.64 mmol) and K₂CO₃ (1.2 g, 9.10 mmol) at room temperature. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford 1-ethyl-5-(2-methyl-4-nitrophenoxy)-1,3-benzodiazole (800.0 mg, crude) as a brown solid. LCMS (ESI, m/z): [M+H]⁺=298.1.

### Step 5. Synthesis of 4-[(1-ethyl-1,3-benzodiazol-5-yl)oxy]-3-methylaniline

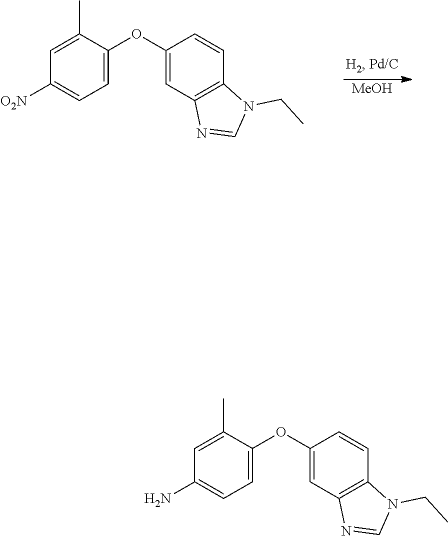 [SMILES: "MeOH" ; CCn1cnc2cc(Oc3ccc([N+](=O)[O-])cc3C)ccc21 ; CCn1cnc2cc(Oc3ccc(N)cc3C)ccc21]

To a solution of 1-ethyl-5-(2-methyl-4-nitrophenoxy)-1,3-benzodiazole (800.0 mg, 2.69 mmol) in MeOH (20.0 mL) was added Pd/C (240.0 mg, 10%) at room temperature under N₂. The resulting mixture was stirred at room temperature for 2 h under H₂. After the reaction was completed, the resulting mixture was filtered. The filtrate was concentrated under reduced pressure to afford 4-[(1-ethyl-1,3-benzodiazol-5-yl)oxy]-3-methylaniline (500.0 mg, crude) as a brown oil. LCMS (ESI, m/z): [M+H]⁺=268.1.

### Step 6. Synthesis of 6-chloro-N-{4-[(1-ethyl-1,3-benzodiazol-5-yl)oxy]-3-methylphenyl}pyrido[3,2-d]pyrimidin-4-amine

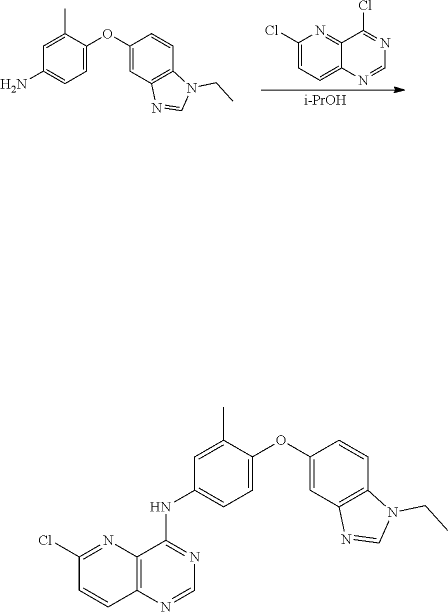 [SMILES: "i-PrOH" ; CCn1cnc2cc(Oc3ccc(N)cc3C)ccc21 ; Clc1ccc2ncnc(Cl)c2n1 ; CCn1cnc2cc(Oc3ccc(Nc4ncnc5ccc(Cl)nc45)cc3C)ccc21]

To a solution of 4-[(1-ethyl-1,3-benzodiazol-5-yl)oxy]-3-methylaniline (250.0 mg, 0.94 mmol) in i-PrOH (15.0 mL) was added 4,6-dichloropyrido[3,2-d]pyrimidine (187.1 mg, 0.94 mmol) at room temperature. The resulting mixture was stirred at room temperature for 2 h. After the reaction was completed, the resulting mixture was concentrated under reduced pressure. The residue was purified by flash column chromatography with CH₂Cl₂/methanol (93/7, v/v) to afford 6-chloro-N-{4-[(1-ethyl-1,3-benzodiazol-5-yl)oxy]-3-methylphenyl}pyrido[3,2-d]pyrimidin-4-amine (181.0 mg, 44%) as a brown oil. LCMS (ESI, m/z): [M+H]⁺=431.1.

### Step 7. Synthesis of (3E)-3-[2-(dimethylamino)ethylidene]-1-[4-({4-[(1-ethyl-1,3-benzodiazol-5-yl)oxy]-3-methylphenyl}amino)pyrido[3,2-d]pyrimidin-6-yl]pyrrolidin-2-one (Compound 65)

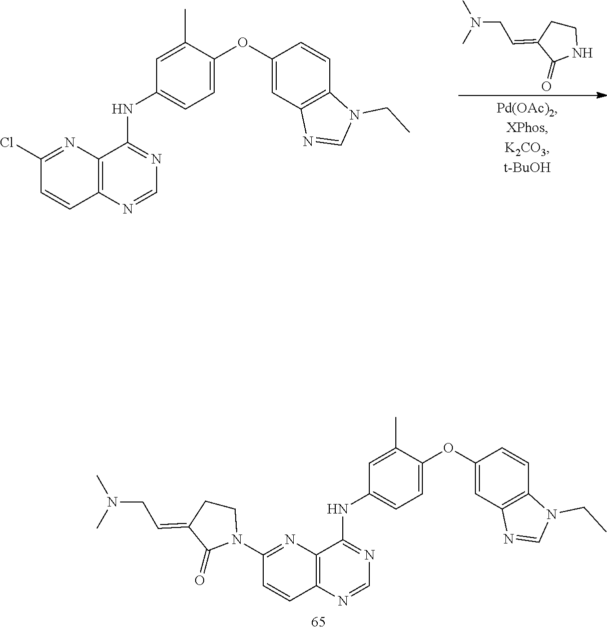

To a solution of 6-chloro-N-{4-[(1-ethyl-1,3-benzodiazol-5-yl)oxy]-3-methylphenyl}pyrido[3,2-d]pyrimidin-4-amine (100.0 mg, 0.23 mmol) in t-BuOH (15.0 mL) was added (3E)-3-[2-(dimethylamino)ethylidene]pyrrolidin-2-one (53.7 mg, 0.35 mmol), XPhos (22.1 mg, 0.05 mmol), K₂CO₃ (96.2 mg, 0.70 mmol) and Pd(OAc)₂ (5.2 mg, 0.02 mmol) at room temperature under N₂. The resulting mixture was stirred at 100° C. for 2 h. After the reaction was completed, the resulting mixture was concentrated under reduced pressure. The residue was purified by flash column chromatography with DCM/MeOH (94/6, v/v) and then purified by Prep-HPLC with the following conditions (Column: XSelect CSH Prep C18 OBD Column, 19×250 mm, 5 μm; Mobile Phase A: Water (10 mmol/L NH₄HCO₃), Mobile Phase B: MeOH-HPLC; Flow rate: 25 mL/min; Gradient: 75% B to 75% B in 12 min; Wave Length: 254 nm) to afford (3E)-3-[2-(dimethylamino)ethylidene]-1-[4-({4-[(1-ethyl-1,3-benzodiazol-5-yl)oxy]-3-methylphenyl}amino)pyrido[3,2-d]pyrimidin-6-yl]pyrrolidin-2-one (Compound 65) (2.1 mg, 1%) as a light yellow solid. LCMS (ESI, m/z): [M+H]⁺=549.4. ¹H NMR (400 MHz, DMSO-d₆): δ 9.51 (s, 1H), 8.97 (d, J=9.2 Hz, 1H), 8.58 (s, 1H), 8.25-8.23 (m, 2H), 7.84-7.77 (m, 2H), 7.62 (d, J=8.4 Hz, 1H), 7.10 (d, J=2.4 Hz, 1H), 7.01-6.98 (m, 1H), 6.92 (d, J=8.8 Hz, 1H), 6.60-6.55 (m, 1H), 4.35-4.25 (m, 4H), 3.11 (d, J=6.8 Hz, 2H), 2.89-2.84 (m, 2H), 2.28 (s, 3H), 2.21 (s, 6H), 1.45-1.41 (m, 3H).

### Example S60: Synthesis of (E)-1-(4-((4-(benzo[d]oxazol-5-yloxy)-3-methylphenyl)amino)pyrido[3,2-d]pyrimidin-6-yl)-3-(2-(dimethylamino)ethylidene)pyrrolidin-2-one (Compound 66)

### Step 1. Synthesis of 2-aminobenzene-1,4-diol

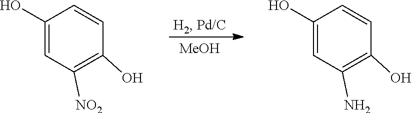 [SMILES: "MeOH" ; O=[N+]([O-])c1cc(O)ccc1O ; "H2, Pd/C" ; Nc1cc(O)ccc1O]

To a solution of 2-nitrobenzene-1,4-diol (500.0 mg, 3.22 mmol) in MeOH (20.0 mL) was added Pd/C (150.0 mg, 10%) at room temperature under N₂. The resulting mixture was stirred at room temperature for 2 h under H₂. After the reaction was completed, the resulting mixture was filtered. The filtrate was concentrated under reduced pressure to afford 2-aminobenzene-1,4-diol (400.0 mg, crude) as a brown oil. LCMS (ESI, m/z): [M+H]⁺=126.0.

### Step 2. Synthesis of benzo[d]oxazol-5-ol

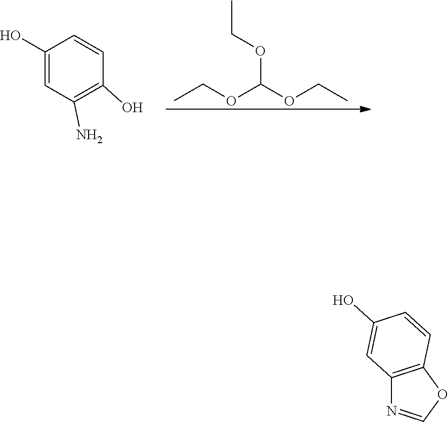

To a solution of 2-aminobenzene-1,4-diol (4.5 g, crude) in triethoxymethane (19.0 mL) was added HCl (0.3 mL) at room temperature. The resulting mixture was stirred at 65° C. for 30 min. After the reaction was completed, the resulting mixture was diluted with water and extracted with ethyl acetate. The combined organic layers were washed aq.NaHCO₃, dried over anhydrous Na₂SO₄ and filtered. The filtrate was concentrated under reduced pressure to afford benzo[d]oxazol-5-ol (4.6 g, crude) as a brown solid. LCMS (ESI, m/z): [M+H]⁺=136.0.

### Step 3. Synthesis of 5-(2-methyl-4-nitrophenoxy)benzo[d]oxazole

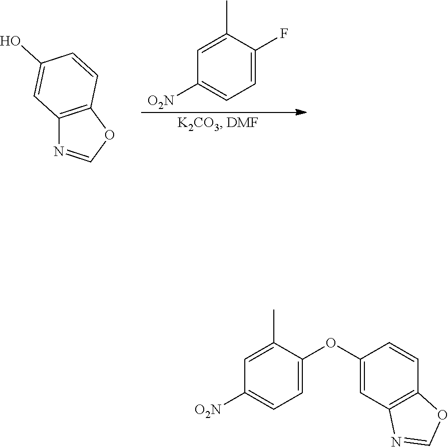

To a solution of benzo[d]oxazol-5-ol (2.0 g, 14.80 mmol) in DMF (50.0 mL) was added 1-fluoro-2-methyl-4-nitrobenzene (6.9 g, 44.40 mmol) and K₂CO₃ (6.1 g, 44.40 mmol) at room temperature. The resulting mixture was stirred at 50° C. for 16 h. After the reaction was completed, the resulting mixture was diluted with water and extracted with ethyl acetate. The combined organic layer was washed with brine, dried over anhydrous sodium sulfate and filtered. The filtrate was concentrated under reduced pressure. The residue was purified by flash chromatography with petroleum ether/EtOAc (7/1, v/v) to afford 5-(2-methyl-4-nitrophenoxy)benzo[d]oxazole (1.5 g, 37%) as a white solid. LCMS (ESI, m/z): [M+H]⁺=271.1.

### Step 4. Synthesis of 4-(benzo[d]oxazol-5-yloxy)-3-methylaniline

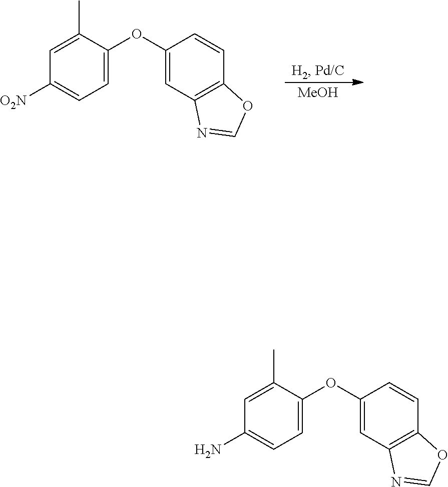 [SMILES: "MeOH" ; Cc1cc([N+](=O)[O-])ccc1Oc1ccc2ocnc2c1 ; "H2, Pd/C" ; Cc1cc(N)ccc1Oc1ccc2ocnc2c1]

To a solution of 5-(2-methyl-4-nitrophenoxy)benzo[d]oxazole (600.0 mg, 2.22 mmol) in MeOH (30.0 mL) was added Pd/C (236.3 mg, 10%) at room temperature under N₂. The resulting mixture was stirred for 4 h at room temperature under H₂. After the reaction was completed, the resulting mixture was filtered. The filtrate was concentrated under reduced pressure. The residue was purified by flash chromatography with petroleum ether/EtOAc (7/3, v/v) to afford 4-(benzo[d]oxazol-5-yloxy)-3-methylaniline (300.0 mg, 56%) as a white solid. LCMS (ESI, m/z): [M+H]⁺=241.1.

### Step 5. Synthesis of N-(4-(benzo[d]oxazol-5-yloxy)-3-methylphenyl)-6-chloropyrido[3,2-d]pyrimidin-4-amine

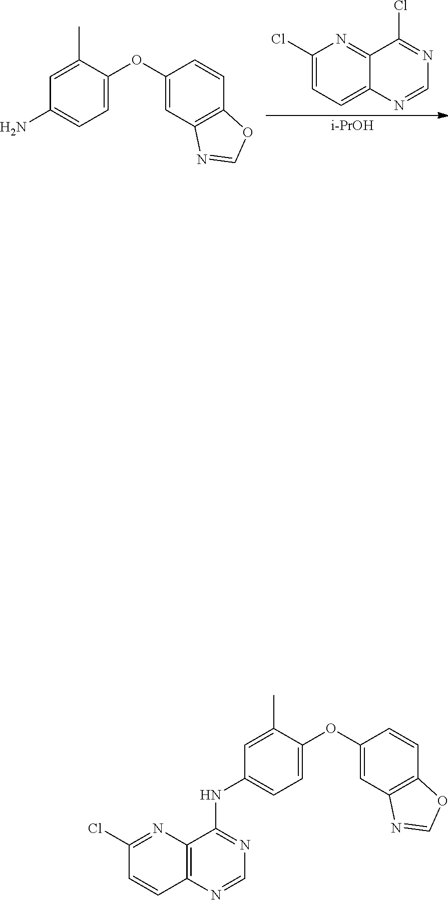 [SMILES: "i-PrOH" ; Cc1cc(N)ccc1Oc1ccc2ocnc2c1 ; Clc1ccc2ncnc(Cl)c2n1 ; Cc1cc(Nc2ncnc3ccc(Cl)nc23)ccc1Oc1ccc2ocnc2c1]

To a solution of 4-(benzo[d]oxazol-5-yloxy)-3-methylaniline (300.0 mg, 1.25 mmol) in isopropyl alcohol (10.0 mL) was added 4,6-dichloropyrido[3,2-d]pyrimidine (249.8 mg, 1.25 mmol) at room temperature under N₂. The resulting mixture was stirred at room temperature for 1 h. After the reaction was completed, the resulting mixture was concentrated under reduced pressure. The residue was purified by flash chromatography with CH₂Cl₂/MeOH (10/1, v/v) to afford N-(4-(benzo[d]oxazol-5-yloxy)-3-methylphenyl)-6-chloropyrido[3,2-d]pyrimidin-4-amine (150.0 mg, 29%) as a yellow oil. LCMS (ESI, m/z): [M+H]⁺=404.1.

### Step 6. Synthesis of (E)-1-(4-((4-(benzo[d]oxazol-5-yloxy)-3-methylphenyl)amino)pyrido[3,2-d]pyrimidin-6-yl)-3-(2-(dimethylamino)ethylidene)pyrrolidin-2-one (Compound 66)

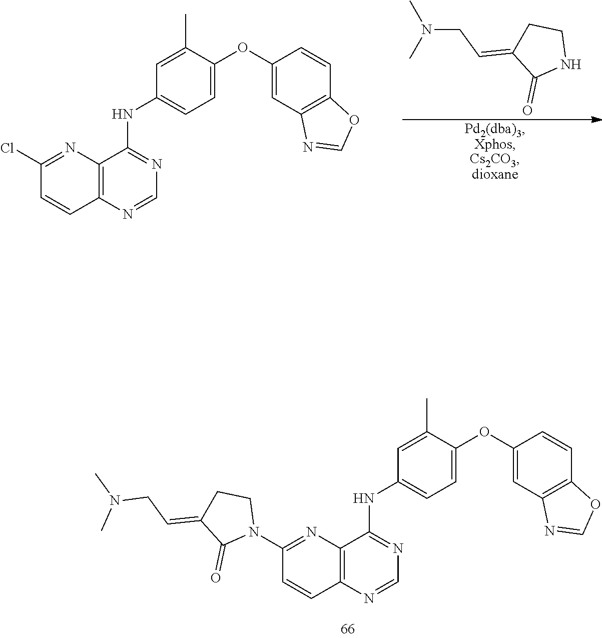 [SMILES: Cc1cc(Nc2ncnc3ccc(Cl)nc23)ccc1Oc1ccc2ocnc2c1 ; CN(C)C/C=C1\CCNC1=O ; "Pd2(dba)3,Xphos,Cs2CO3,dioxane" ; Cc1cc(Nc2ncnc3ccc(N4CC/C(=C\CN(C)C)C4=O)nc23)ccc1Oc1ccc2ocnc2c1]

To a solution of N-(4-(benzo[d]oxazol-5-yloxy)-3-methylphenyl)-6-chloropyrido[3,2-d]pyrimidin-4-amine (111.0 mg, 0.28 mmol) in dioxane (4.0 mL) was added (E)-3-(2-(dimethylamino)ethylidene)pyrrolidin-2-one (42.4 mg, 0.28 mmol), XPhos (26.2 mg, 0.06 mmol), Cs₂CO₃ (268.7 mg, 0.83 mmol) and Pd₂(dba)₃ (25.2 mg, 0.03 mmol) at room temperature under N₂. The resulting mixture was stirred at 100° C. for 2 h under N₂. After the reaction was completed, the reaction was diluted with water and extracted with ethyl acetate. The combined organic layer was washed with brine, dried over anhydrous Na₂SO₄ and filtered. The filtrate was concentrated under reduced pressure. The residue was purified by Prep-HPLC with the following conditions (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 40% B to 60% B in 8 min; Wave Length: 254 nm) to afford (E)-1-(4-((4-(benzo[d]oxazol-5-yloxy)-3-methylphenyl)amino)pyrido[3,2-d]pyrimidin-6-yl)-3-(2-(dimethylamino)ethylidene)pyrrolidin-2-one (Compound 66) (7.0 mg, 1%) as a yellow solid. LCMS (ESI, m/z): [M+H]⁺=522.4. ¹H NMR (400 MHz, DMSO-d₆): δ 9.53 (s, 1H), 8.97 (d, J=9.2 Hz, 1H), 8.75 (s, 1H), 8.60 (s, 1H), 8.25 (d, J=9.2 Hz, 1H), 7.87-7.84 (m, 2H), 7.78 (d, J=8.8 Hz, 1H), 7.25 (d, J=2.4 Hz, 1H), 7.12-7.09 (m, 1H), 7.01 (d, J=8.4 Hz, 1H), 6.61-6.55 (m, 1H), 4.36-4.32 (m, 2H), 3.11 (d, J=6.8 Hz, 2H), 2.91-2.85 (m, 2H), 2.22-2.08 (m, 9H).

### Example S61: Synthesis of (E)-1-(4-((4-(benzo[d]thiazol-5-yloxy)-3-methylphenyl)amino)pyrido[3,2-d]pyrimidin-6-yl)-3-(2-(dimethylamino)ethylidene)pyrrolidin-2-one (Compound 67)

### Step 1. Synthesis of benzo[d]thiazol-5-ol

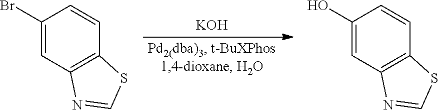 [SMILES: Brc1ccc2scnc2c1 ; "KOH" ; "Pd2(dba)3, t-BuXPhos1,4-dioxane, H2O" ; Oc1ccc2scnc2c1]

To a solution of 5-bromobenzo[d]thiazole (5.0 g, 23.58 mmol) in dioxane/H₂O (50.0/10.0 mL) was added KOH (5.3 g, 94.34 mmol), Pd₂(dba)₃ (2.2 g, 2.36 mmol) and t-BuXPhos (2.0 g, 4.72 mmol) at room temperature under N₂. The resulting mixture was stirred at 100° C. for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (45/55, v/v) to afford benzo[d]thiazol-5-ol (700.0 mg, 20%) as a brown solid. LCMS (ESI, m/z): [M+H]⁺=152.0.

### Step 2. Synthesis of 5-(2-methyl-4-nitrophenoxy)benzo[d]thiazole

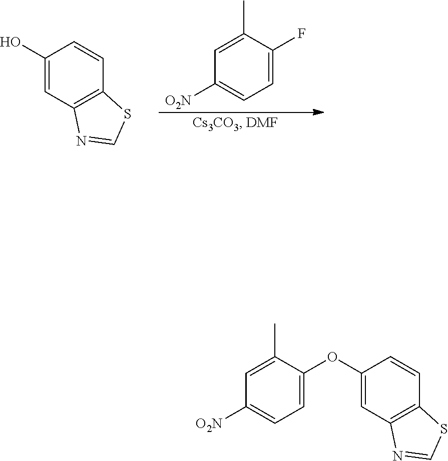

To a solution of benzo[d]thiazol-5-ol (700.0 mg, 4.64 mmol) in DMF (7.0 mL) was added 1-fluoro-2-methyl-4-nitrobenzene (718.5 mg, 4.64 mmol) and Cs₂CO₃ (3.0 g, 9.27 mmol) at room temperature. The resulting mixture was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (78/22, v/v) to afford 5-(2-methyl-4-nitrophenoxy)benzo[d]thiazole (580.0 mg, 44%) as a brown solid. LCMS (ESI, m/z): [M+H]⁺=287.0.

### Step 3. Synthesis of 4-(benzo[d]thiazol-5-yloxy)-3-methylaniline

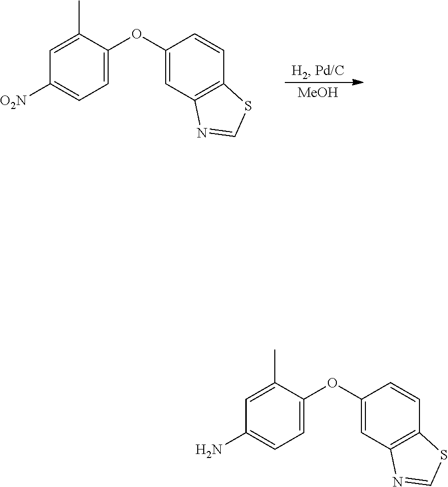 [SMILES: "MeOH" ; Cc1cc([N+](=O)[O-])ccc1Oc1ccc2scnc2c1 ; "H2, Pd/C" ; Cc1cc(N)ccc1Oc1ccc2scnc2c1]

To a solution of 5-(2-methyl-4-nitrophenoxy)benzo[d]thiazole (580.0 mg, 2.03 mmol) in MeOH (10.0 mL) was added Pd/C (150.0 mg, 10%) at room temperature under N₂. The mixture was stirred at room temperature for 2 h under H₂. After the reaction was completed, the reaction mixture was filtered. The filtrate was concentrated under reduced pressure to afford 4-(benzo[d]thiazol-5-yloxy)-3-methylaniline (300.0 mg, crude) as a brown oil. LCMS (ESI, m/z): [M+H]⁺=257.1.

### Step 4. Synthesis of N-(4-(benzo[d]thiazol-5-yloxy)-3-methylphenyl)-6-chloropyrido[3,2-d]pyrimidin-4-amine

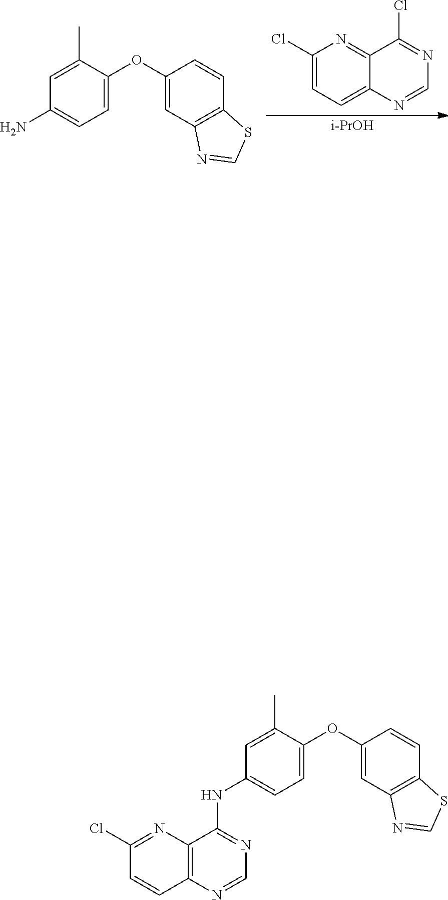 [SMILES: "i-PrOH" ; Cc1cc(N)ccc1Oc1ccc2scnc2c1 ; Clc1ccc2ncnc(Cl)c2n1 ; Cc1cc(Nc2ncnc3ccc(Cl)nc23)ccc1Oc1ccc2scnc2c1]

To a solution of 4-(benzo[d]thiazol-5-yloxy)-3-methylaniline (270.0 mg, crude) in i-PrOH (10.0 mL) was added 4,6-dichloropyrido[3,2-d]pyrimidine (210.7 mg, 1.05 mmol) at room temperature. The mixture was stirred at room temperature for 2 h. After the reaction was completed, the mixture was concentrated under reduced pressure. The residue was purified by flash column chromatography with CH₂Cl₂/MeOH (89/11, v/v) to afford N-(4-(benzo[d]thiazol-5-yloxy)-3-methylphenyl)-6-chloropyrido[3,2-d]pyrimidin-4-amine (170.0 mg, 38%) as a yellow solid. LCMS (ESI, m/z): [M+H]⁺=420.1.

### Step 5. Synthesis of (E)-1-(4-((4-(benzo[d]thiazol-5-yloxy)-3-methylphenyl)amino)pyrido[3,2-d]pyrimidin-6-yl)-3-(2-(dimethylamino)ethylidene)pyrrolidin-2-one (Compound 67)

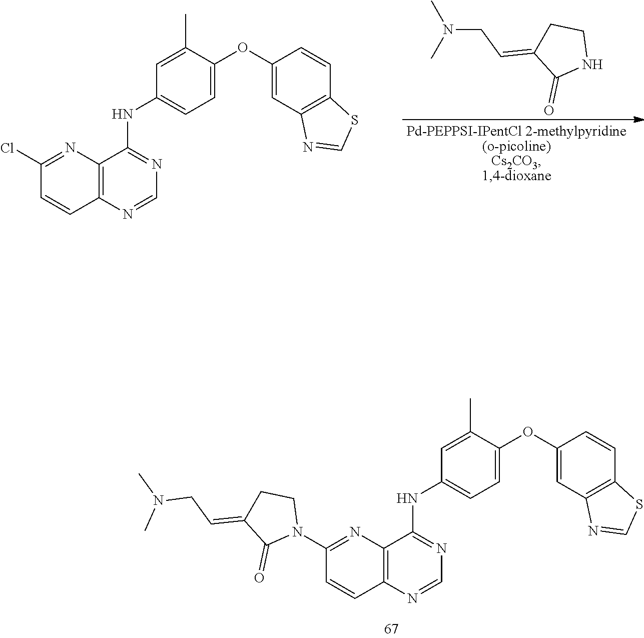 [SMILES: Cc1cc(Nc2ncnc3ccc(Cl)nc23)ccc1Oc1ccc2scnc2c1 ; CN(C)C/C=C1\CCNC1=O ; "Pd-PEPPSI-IPentCl 2-methylpyridine(o-picoline)Cs2CO3,1,4-dioxane" ; Cc1cc(Nc2ncnc3ccc(N4CC/C(=C\CN(C)C)C4=O)nc23)ccc1Oc1ccc2scnc2c1]

To a solution of N-(4-(benzo[d]thiazol-5-yloxy)-3-methylphenyl)-6-chloropyrido[3,2-d]pyrimidin-4-amine (100.0 mg, 0.24 mmol) in 1,4-dioxane (5.0 mL) was added (E)-3-(2-(dimethylamino)ethylidene)pyrrolidin-2-one (55.1 mg, 0.36 mmol), Cs₂CO₃ (232.8 mg, 0.71 mmol) and Pd-PEPPSI-IPentCl 2-methylpyridine (o-picoline) (20.0 mg, 0.02 mmol) at room temperature under N₂. The resulting mixture was stirred at 100° C. for 2 h. After the reaction was completed, the mixture was filtered. The filtrate was concentrated under reduced pressure. The residue was purified by flash column chromatography with CH₂Cl₂/MeOH (89/11, v/v) and then purified by Prep-HPLC with the following conditions: (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 40% B to 60% B in 8 min; Wave Length: 254 nm) to afford (E)-1-(4-((4-(benzo[d]thiazol-5-yloxy)-3-methylphenyl)amino)pyrido[3,2-d]pyrimidin-6-yl)-3-(2-(dimethylamino)ethylidene)pyrrolidin-2-one (Compound 67) (13.4 mg, 10%) as a yellow solid. LCMS (ESI, m/z): [M+H]⁺=538.3. ¹H NMR (400 MHz, DMSO-d₆): δ 9.57 (s, 1H), 9.41 (s, 1H), 8.98 (d, J=9.2 Hz, 1H), 8.62 (s, 1H), 8.26 (d, J=9.2 Hz, 1H), 8.16 (d, J=8.8 Hz, 1H), 7.92-7.88 (m, 2H), 7.43 (d, J=2.4 Hz, 1H), 7.27-7.19 (m, 1H), 7.12-7.08 (m, 1H), 6.61-6.54 (m, 1H), 4.38-4.34 (m, 2H), 3.11 (d, J=6.8 Hz, 2H), 2.91-2.85 (m, 2H), 2.25 (s, 3H), 2.21 (s, 6H).

### Example S62: Synthesis of (3E)-3-[2-(dimethylamino)ethylidene]-1-[4-({3-methyl-4-[(1-methyl-1,2,3-benzotriazol-5 yl)oxy]phenyl}amino)pyrido[3,2-d]pyrimidin-6-yl]pyrrolidin-2-one (Compound 68)

### Step 1. Synthesis of 1-methyl-1,2,3-benzotriazol-5-ol

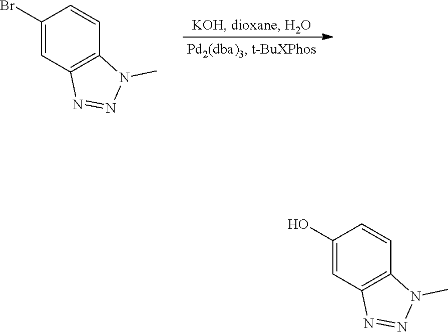

To a solution of 5-bromo-1-methyl-1,2,3-benzotriazole (1.0 g, 4.72 mmol) in dioxane (15.0 mL) and H₂O (3.0 mL) was added KOH (1.1 g, 18.86 mmol), Pd₂(dba)₃ (0.4 g, 0.47 mmol) and t-BuXPhos (0.4 g, 0.94 mmol) at room temperature under N₂. The resulting mixture was stirred at 100° C. for 4 h under N₂. After the reaction was completed, the mixture was diluted with H₂O and extracted with ethyl acetate. The combined organic layer was dried over anhydrous sodium sulfate and filtered. The filtrate was concentrated under reduced pressure. The residue was purified by flash column chromatography with dichloromethane/methanol (90/10, v/v) to afford 1-methyl-1,2,3-benzotriazol-5-ol (500.0 mg, 71%) as a pink solid. LCMS (ESI, m/z): [M+H]⁺=150.1.

### Step 2. Synthesis of 1-methyl-5-(2-methyl-4-nitrophenoxy)-1,2,3-benzotriazole

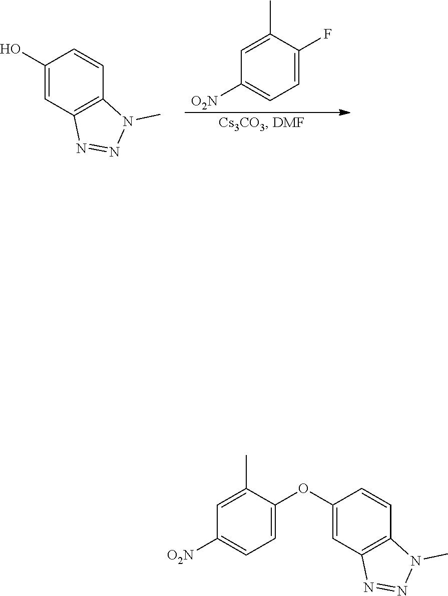

To a solution of 1-methyl-1,2,3-benzotriazol-5-ol (430.0 mg, 2.88 mmol) in DMF (10.0 mL) was added K₂CO₃ (796.9 mg, 5.77 mmol) and 1-fluoro-2-methyl-4-nitrobenzene (447.2 mg, 2.88 mmol) at room temperature. The mixture was stirred at 50° C. for 16 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with dichloromethane/methanol (95/5, v/v) to afford 1-methyl-5-(2-methyl-4-nitrophenoxy)-1,2,3-benzotriazole (770.0 mg, 93%) as a white solid. LCMS (ESI, m/z): [M+H]⁺=285.1.

### Step 3. Synthesis of 3-methyl-4-[(1-methyl-1,2,3-benzotriazol-5-yl)oxy]aniline

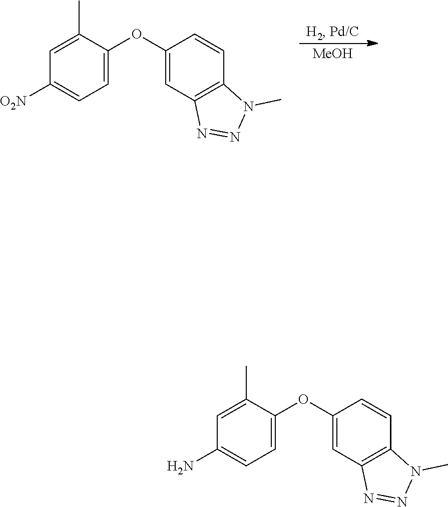 [SMILES: "MeOH" ; Cc1cc([N+](=O)[O-])ccc1Oc1ccc2c(c1)nnn2C ; "H2, Pd/C" ; Cc1cc(N)ccc1Oc1ccc2c(c1)nnn2C]

To a solution of 1-methyl-5-(2-methyl-4-nitrophenoxy)-1,2,3-benzotriazole (770.0 mg, 2.71 mmol) in MeOH (15.0 ML) was added Pd/C (150.0 mg, 10%) at room temperature under N₂. The resulting mixture was stirred at room temperature for 4 h under H₂. After the reaction was completed, the resulting mixture was filtered. The filtrate was concentrated under reduced pressure to afford 3-methyl-4-[(1-methyl-1,2,3-benzotriazol-5-yl)oxy]aniline (748.0 mg, crude) as a pink solid. LCMS (ESI, m/z): [M+H]⁺=255.1.

### Step 4. Synthesis of 6-chloro-N-{3-methyl-4-[(1-methyl-1,2,3-benzotriazol-5-yl)oxy]phenyl}pyrido[3,2-d]pyrimidin-4-amine

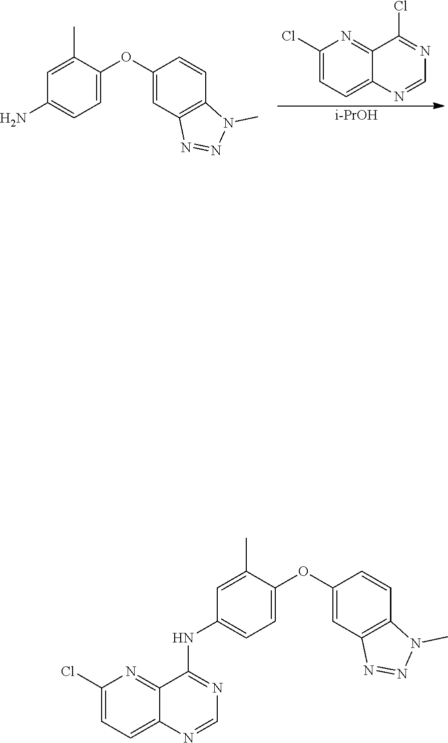 [SMILES: "i-PrOH" ; Cc1cc(N)ccc1Oc1ccc2c(c1)nnn2C ; Clc1ccc2ncnc(Cl)c2n1 ; Cc1cc(Nc2ncnc3ccc(Cl)nc23)ccc1Oc1ccc2c(c1)nnn2C]

To a solution of 3-methyl-4-[(1-methyl-1,2,3-benzotriazol-5-yl)oxy]aniline (670.0 mg, 2.64 mmol) in isopropanol (10.0 mL) was added 4,6-dichloropyrido[3,2-d]pyrimidine (527.0 mg, 2.64 mmol) at room temperature. The resulting mixture was stirred at room temperature for 16 h. After the reaction was completed, the resulting mixture was concentrated under reduced pressure. The residue was purified by reverse phase flash column chromatography with acetonitrile/water (60/40, v/v) to afford 6-chloro-N-{3-methyl-4-[(1-methyl-1,2,3-benzotriazol-5-yl)oxy]phenyl}pyrido[3,2-d]pyrimidin-4-amine (978.9 mg, 89%) as a yellow solid. LCMS (ESI, m/z): [M+H]⁺=418.1.

### Step 5. Synthesis of (3E)-3-[2-(dimethylamino)ethylidene]-1-[4-({3-methyl-4-[(1-methyl-1,2,3-benzotriazol-5-yl)oxy]phenyl}amino)pyrido[3,2-d]pyrimidin-6-yl]pyrrolidin-2-one (Compound 68)

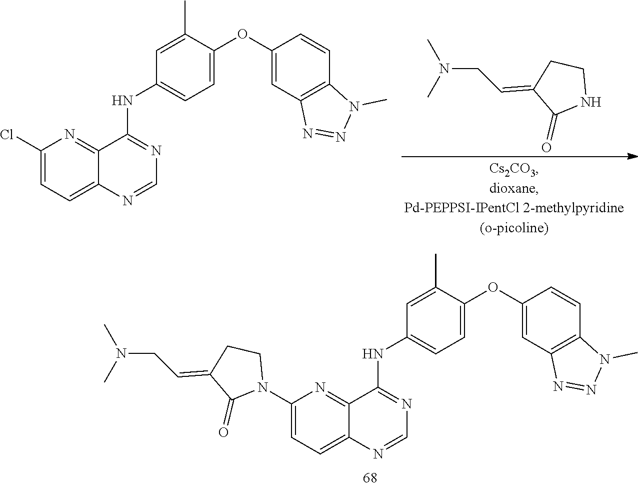

To a solution of 6-chloro-N-{3-methyl-4-[(1-methyl-1,2,3-benzotriazol-5-yl)oxy]phenyl}pyrido[3,2-d]pyrimidin-4-amine (250.0 mg, 0.60 mmol) in 1,4-dioxane (5.0 mL) was added Cs₂CO₃ (584.8 mg, 1.79 mmol), Pd-PEPPSI-IPentCl 2-methylpyridine (o-picoline) (50.3 mg, 0.06 mmol) and (3E)-3-[2-(dimethylamino)ethylidene]pyrrolidin-2-one (110.7 mL, 0.72 mmol) at room temperature under N₂. The resulting mixture was stirred at 100° C. for 2 h under N₂. After the reaction was completed, the mixture was cooled to room temperature and filtered. The filtrate was concentrated under vacuum. The residue was purified by flash chromatography with CH₂Cl₂/MeOH (85/15, v/v) and then purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 35% B to 45% B in 8 min; Wave Length: 254 nm) to afford (3E)-3-[2-(dimethylamino)ethylidene]-1-[4-({3-methyl-4-[(1-methyl-1,2,3-benzotriazol-5-yl)oxy]phenyl}amino)pyrido[3,2-d]pyrimidin-6-yl]pyrrolidin-2-one (Compound 68) (34.5 mg, 10%) as a white solid. LCMS (ESI, m/z): [M+H]⁺=536.4. ¹H NMR (400 MHz, DMSO-d₆): δ 9.57 (s, 1H), 8.99 (d, J=9.2 Hz, 1H), 8.62 (s, 1H), 8.27 (d, J=9.2 Hz, 1H), 7.91-7.86 (m, 3H), 7.38-7.35 (m, 1H), 7.28 (d, J=2.0 Hz, 1H), 7.04 (d, J=8.4 Hz, 1H), 6.59-6.56 (m, 1H), 4.38-4.31 (m, 5H), 3.12 (d, J=6.8 Hz, 2H), 2.91-2.86 (m, 2H), 2.26-2.22 (m, 9H).

### Example S63: Synthesis of (3E)-3 [2-(dimethylamino)ethylidene]-1-[4-({2-fluoro-3-methyl-4-[(1-methyl-1,3-benzodiazol-5 yl)oxy]phenyl}amino)pyrido[3,2-d]pyrimidin-6-yl]pyrrolidin-2-one (Compound 69)

### Step 1. Synthesis of 6-chloro-N-{2-fluoro-3-methyl-4-[(1-methyl-1,3-benzodiazol-5-yl)oxy]phenyl}pyrido[3,2-d]pyrimidin-4-amine

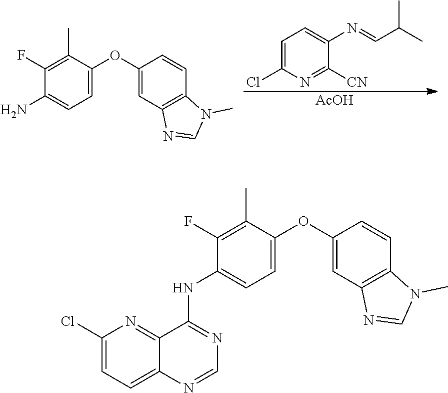

To a stirred solution of 2-fluoro-3-methyl-4-[(1-methyl-1,3-benzodiazol-5-yl)oxy]aniline (250.0 mg, 0.92 mmol) in AcOH (8.0 mL) was added (E)-N′-(6-chloro-2-cyanopyridin-3-yl)-N,N-dimethylmethanimidamide (192.3 mg, 0.92 mmol) at room temperature. The resulting mixture was stirred at 85° C. for 2 h. After the reaction was completed, the resulting mixture was concentrated under reduced pressure. The residue was purified by flash column chromatography with CH₂Cl₂/MeOH (5/1, v/v) to afford 6-chloro-N-{2-fluoro-3-methyl-4-[(1-methyl-1,3-benzodiazol-5-yl)oxy]phenyl}pyrido[3,2-d]pyrimidin-4-amine (300.0 mg, 74%) as a brown solid. LCMS (ESI, m/z): [M+H]⁺=435.1.

### Step 2. Synthesis of (3E)-3-[2-(dimethylamino)ethylidene]-1-[4-({2-fluoro-3-methyl-4-[(1-methyl-1,3-benzodiazol-5-yl)oxy]phenyl}amino)pyrido[3,2-d]pyrimidin-6-yl]pyrrolidin-2-one (Compound 69)

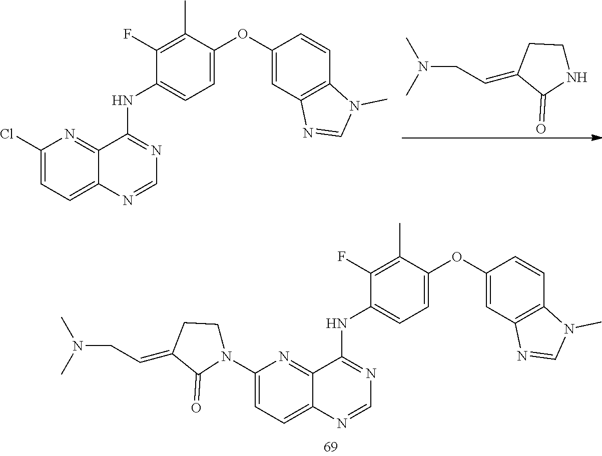

To a stirred mixture of 6-chloro-N-{2-fluoro-3-methyl-4-[(1-methyl-1,3-benzodiazol-5-yl)oxy]phenyl}pyrido[3,2-d]pyrimidin-4-amine (220.0 mg, 0.51 mmol) and (3E)-3-[2-(dimethylamino)ethylidene]pyrrolidin-2-one (117.0 mg, 0.76 mmol) in dioxane (8.0 mL) were added Cs₂CO₃ (494.5 mg, 1.52 mmol), XPhos (48.2 mg, 0.10 mmol) and Pd(OAc)₂ (22.7 mg, 0.1 mmol) at room temperature. The resulting mixture was stirred at 100° C. for 16 h under N₂. After the reaction was completed, the resulting mixture was diluted with H₂O and extracted with EtOAc. The combined organic layers were washed with brine, dried over anhydrous Na₂SO₄ and filtered. The filtrate was concentrated under reduced pressure. The residue was purified by flash column chromatography with CH₂Cl₂/MeOH (5/1, v/v) and then purified by Prep-HPLC with the following conditions (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 35% B to 50% B in 8 min; Wave Length: 254 nm) to afford (3E)-3-[2-(dimethylamino)ethylidene]-1-[4-({2-fluoro-3-methyl-4-[(1-methyl-1,3-benzodiazol-5-yl)oxy]phenyl}amino)pyrido[3,2-d]pyrimidin-6-yl]pyrrolidin-2-one (Compound 69) (16.4 mg, 5%) as a yellow solid. LCMS (ESI, m/z): [M+H]⁺=553.3. ¹H NMR (400 MHz, DMSO-d₆): δ 9.52 (s, 1H), 8.99 (d, J=9.2 Hz, 1H), 8.55 (s, 1H), 8.27 (d, J=9.2 Hz, 1H), 8.21 (s, 1H), 7.77-7.73 (m, 1H), 7.62 (d, J=8.8 Hz, 1H), 7.23 (d, J=2.0 Hz, 1H), 7.08-7.05 (m, 1H), 6.72 (d, J=8.8 Hz, 1H), 6.59-6.55 (m, 1H), 4.29-4.26 (m, 2H), 3.87 (s, 3H), 3.11 (d, J=6.8 Hz, 2H), 2.91-2.85 (m, 2H), 2.34 (s, 3H), 2.22 (s, 6H).

### Example S64: Synthesis of (3E)-3 [2-(dimethylamino)ethylidene]-1-[4-({2-fluoro-5-methyl-4-[(1-methyl-1,3-benzodiazol-5 yl)oxy]phenyl}amino)pyrido[3,2-d]pyrimidin-6-yl]pyrrolidin-2-one (Compound 70)

### Step 1. Synthesis of 5-(5-fluoro-2-methyl-4-nitrophenoxy)-1-methyl-1,3-benzodiazole

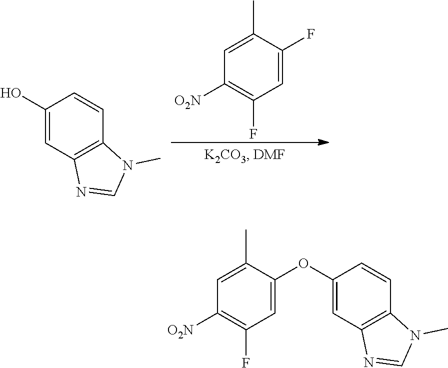 [SMILES: Cn1cnc2cc(O)ccc21 ; Cc1cc([N+](=O)[O-])c(F)cc1F ; Cc1cc([N+](=O)[O-])c(F)cc1Oc1ccc2c(c1)ncn2C]

To a solution of 1-methyl-1,3-benzodiazol-5-ol (1.5 g, 10.12 mmol) in DMF (15.0 mL) was added 1,5-difluoro-2-methyl-4-nitrobenzene (2.1 g, 12.15 mmol) and K₂CO₃ (4.2 g, 30.37 mmol) at room temperature. The resulting mixture was stirred at room temperature for 4 h. After the reaction was completed, the resulting mixture was diluted with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (70/30, v/v) to afford 5-(5-fluoro-2-methyl-4-nitrophenoxy)-1-methyl-1,3-benzodiazole (600.0 mg, 19%) as a yellow solid. LCMS (ESI, m/z): [M+H]⁺=302.1.

### Step 2. Synthesis of 2-fluoro-5-methyl-4-[(1-methyl-1,3-benzodiazol-5-yl)oxy]aniline

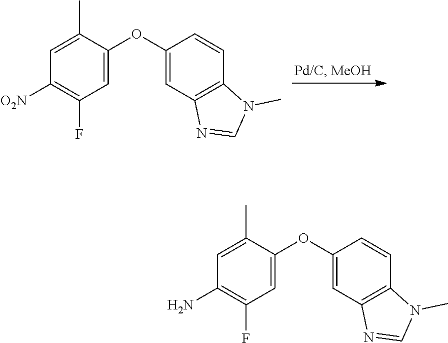 [SMILES: Cc1cc([N+](=O)[O-])c(F)cc1Oc1ccc2c(c1)ncn2C ; Cc1cc(N)c(F)cc1Oc1ccc2c(c1)ncn2C]

To a solution of 5-(5-fluoro-2-methyl-4-nitrophenoxy)-1-methyl-1,3-benzodiazole (600.0 mg, 1.99 mmol) in MeOH (10.0 mL) was added Pd/C (180.0 mg, 10%) at room temperature under N₂. The resulting mixture was stirred at room temperature for 1 h under H₂.

After the reaction was completed, the reaction mixture was filtered. The filtrate was evaporated in vacuo to afford 2-fluoro-5-methyl-4-[(1-methyl-1,3-benzodiazol-5-yl)oxy]aniline (500.0 mg, crude) as a yellow oil. LCMS (ESI, m/z): [M+H]⁺=272.1.

### Step 3. Synthesis of 6-chloro-N-{2-fluoro-5-methyl-4-[(1-methyl-1,3-benzodiazol-5-yl)oxy]phenyl}pyrido[3,2-d]pyrimidin-4-amine

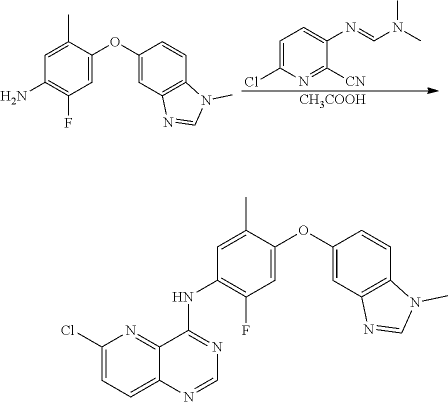

To a solution of 2-fluoro-5-methyl-4-[(1-methyl-1,3-benzodiazol-5-yl)oxy]aniline (250.0 mg, 0.92 mmol) in AcOH (3.0 mL) was added (E)-N′-(6-chloro-2-cyanopyridin-3-yl)-N,N-dimethylmethanimidamide (192.3 mg, 0.92 mmol) at room temperature. The resulting mixture was stirred at 85° C. for 3 h. After the reaction was completed, the resulting mixture was concentrated under reduced pressure. The residue was purified by flash column chromatography with CH₂Cl₂/MeOH (10/1, v/v) to afford 6-chloro-N-{2-fluoro-5-methyl-4-[(1-methyl-1,3-benzodiazol-5-yl)oxy]phenyl}pyrido[3,2-d]pyrimidin-4-amine (200.0 mg, 49%) as a brown oil. LCMS (ESI, m/z): [M+H]⁺=435.1.

### Step 4. Synthesis of (3E)-3-[2-(dimethylamino)ethylidene]-1-[4-({2-fluoro-5-methyl-4-[(1-methyl-1,3-benzodiazol-5-yl)oxy]phenyl}amino)pyrido[3,2-d]pyrimidin-6-yl]pyrrolidin-2-one (Compound 70)

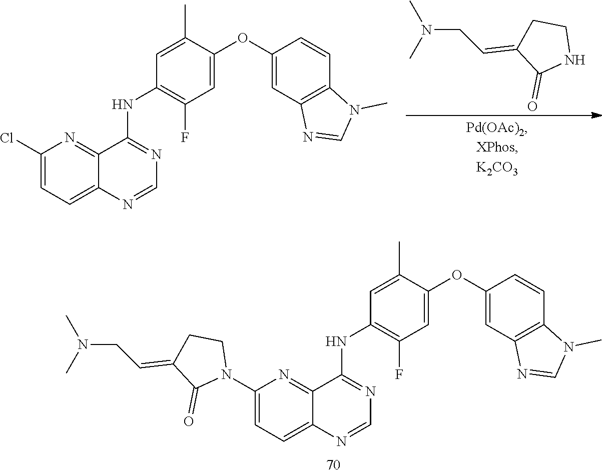

To a solution of 6-chloro-N-{2-fluoro-5-methyl-4-[(1-methyl-1,3-benzodiazol-5-yl)oxy]phenyl}pyrido[3,2-d]pyrimidin-4-amine (200.0 mg, 0.46 mmol) in 1,4-dioxane (3.0 mL) were added (3E)-3-[2-(dimethylamino)ethylidene]pyrrolidin-2-one (70.9 mg, 0.46 mmol), K₂CO₃ (127.1 mg, 0.92 mmol), Pd(OAc)₂ (10.3 mg, 0.05 mmol) and XPhos (43.85 mg, 0.09 mmol) at room temperature under N₂. The mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) and then purified by Prep-HPLC with the following conditions: (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 35% B to 50% B in 8 min; Wave Length: 254 nm) to afford (3E)-3-[2-(dimethylamino)ethylidene]-1-[4-({2-fluoro-5-methyl-4-[(1-methyl-1,3-benzodiazol-5-yl)oxy]phenyl}amino)pyrido[3,2-d]pyrimidin-6-yl]pyrrolidin-2-one (Compound 70) (9.7 mg, 3%) as a light yellow solid. LCMS (ESI, m/z): [M+H]⁺=553.2. ¹H NMR (400 MHz, DMSO-d₆): δ 9.52 (s, 1H), 8.98 (d, J=9.2 Hz, 1H), 8.54 (s, 1H), 8.27-8.22 (m, 2H), 7.81 (d, J=8.8 Hz, 1H), 7.63 (d, J=8.8 Hz, 1H), 7.24 (d, J=2.4 Hz, 1H), 7.08-7.05 (m, 1H), 6.74 (d, J=11.6 Hz, 1H), 6.58-6.55 (m, 1H), 4.28-4.25 (m, 2H), 3.87 (s, 3H), 3.11-3.09 (m, 2H), 2.88-2.84 (m, 2H), 2.29 (s, 3H), 2.21 (s, 6H)

### Example S65: Synthesis of (3E)-3 [2-(dimethylamino)ethylidene]-1-{4-[(4-[imidazo[1,2-a]pyridin-7-yloxy}-3-methylphenyl)amino]pyrido[3,2-d]pyrimidin-6-yl}pyrrolidin-2-one (Compound 71)

### Step 1. Synthesis of 6-chloro-N-(4-{imidazo[1,2-a]pyridin-7-yloxy}-3-methylphenyl)pyrido[3,2-d]pyrimidin-4-amine

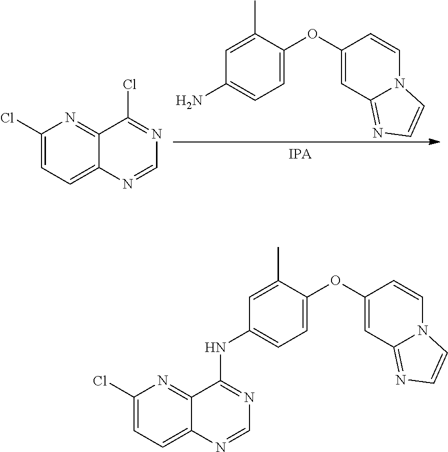 [SMILES: Clc1ccc2ncnc(Cl)c2n1 ; Cc1cc(N)ccc1Oc1ccn2ccnc2c1 ; Cc1cc(Nc2ncnc3ccc(Cl)nc23)ccc1Oc1ccn2ccnc2c1]

To a solution of 4-{imidazo[1,2-a]pyridin-7-yloxy}-3-methylaniline (478.5 mg, 2.00 mmol) in IPA (15.0 mL) was added 4,6-dichloropyrido[3,2-d]pyrimidine (400.0 mg, 2.00 mmol) at 0° C. The resulting mixture was stirred at room temperature for 2 h. After the reaction was completed, the mixture was evaporated in vacuo. The residue was purified by flash column chromatography with CH₂Cl₂/MeOH (9/1, v/v) to afford 6-chloro-N-(4-{imidazo[1,2-a]pyridin-7-yloxy}-3-methylphenyl)pyrido[3,2-d]pyrimidin-4-amine (150.0 mg, 37%) as a white solid. LCMS (ESI, m/z): [M+H]⁺=403.1.

### Step 2. Synthesis of (3E)-3-[2-(dimethylamino)ethylidene]-1-{4-[(4-{imidazo[1,2-a]pyridin-7-yloxy}-3-methylphenyl)amino]pyrido[3,2-d]pyrimidin-6-yl}pyrrolidin-2-one (Compound 71)

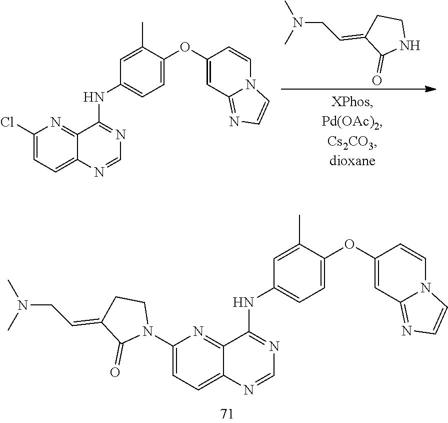

To a solution 6-chloro-N-(4-{imidazo[1,2-a]pyridin-7-yloxy}-3-methylphenyl)pyrido[3,2-d]pyrimidin-4-amine (150.0 mg, 0.37 mmol) in dioxane (25.0 mL) were added (3E)-3-[2-(dimethylamino)ethylidene]pyrrolidin-2-one (68.9 mg, 0.44 mmol), Cs₂CO₃ (303.3 mg, 0.93 mmol), XPhos (35.5 mg, 0.07 mmol) and Pd(OAc)₂ (8.4 mg, 0.04 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions Column: (XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 40% B to 40% B in 8 min; Wave Length: 254 nm) to afford (3E)-3-[2-(dimethylamino)ethylidene]-1-{4-[(4-{imidazo[1,2-a]pyridin-7-yloxy}-3-methylphenyl)amino]pyrido[3,2-d]pyrimidin-6-yl}pyrrolidin-2-one (Compound 71) (7.9 mg, 4%) as a white solid. LCMS (ESI, m/z): [M+H]⁺=521.3. ¹H NMR (400 MHz, DMSO-d₆): δ 9.59 (s, 1H), 9.00-8.97 (m, 1H), 8.63-8.54 (m, 2H), 8.28-8.25 (m, 1H), 7.93-7.84 (m, 3H), 7.43 (d, J=4.8 Hz, 1H), 7.20-7.17 (m, 1H), 6.82-6.79 (m, 1H), 6.61-6.56 (m, 2H), 4.41-4.35 (m, 2H), 3.15-3.10 (m, 2H), 2.91-2.85 (m, 2H), 2.23-2.20 (m, 9H).

### Example S66: Synthesis of (E)-3-(2-(dimethylamino)ethylidene)-1-(4-((4-((6-ethylpyridin-3-yl)oxy)-3-methylphenyl)amino)pyrido[3,2-d]pyrimidin-6-yl)pyrrolidin-2-one (Compound 72)

### Step 1. Synthesis of 6-chloro-N-(4-((6-ethylpyridin-3-yl)oxy)-3-methylphenyl)pyrido[3,2-d]pyrimidin-4-amine

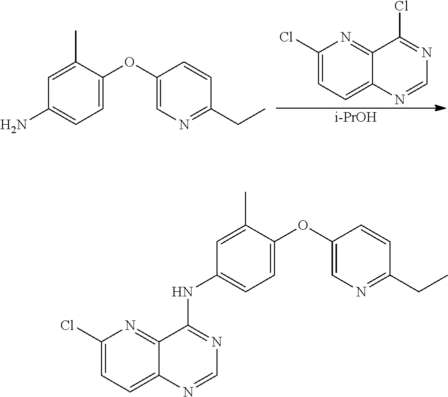 [SMILES: "i-PrOH" ; CCc1ccc(Oc2ccc(N)cc2C)cn1 ; Clc1ccc2ncnc(Cl)c2n1 ; CCc1ccc(Oc2ccc(Nc3ncnc4ccc(Cl)nc34)cc2C)cn1]

### Step 2. Synthesis of (E)-3-(2-(dimethylamino)ethylidene)-1-(4-((4-((6-ethylpyridin-3-yl)oxy)-3-methylphenyl)amino)pyrido[3,2-d]pyrimidin-6-yl)pyrrolidin-2-one (Compound 72)

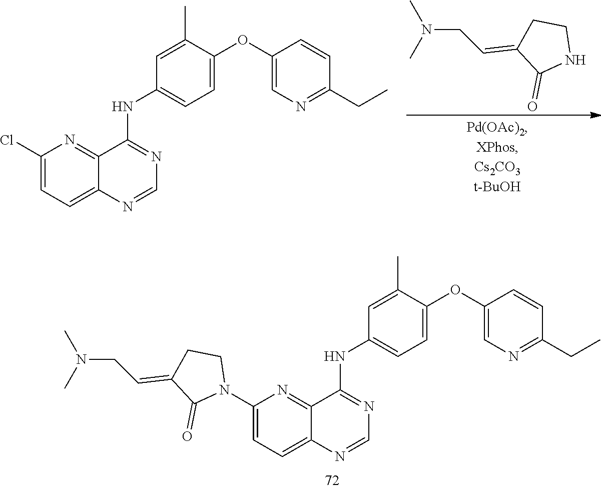

To a solution of 6-chloro-N-(4-((6-ethylpyridin-3-yl)oxy)-3-methylphenyl)pyrido[3,2-d]pyrimidin-4-amine (150.0 mg, 0.38 mmol) in t-BuOH (4.0 mL) was added (E)-3-(2-(dimethylamino)ethylidene)pyrrolidin-2-one (88.6 mg, 0.57 mmol), XPhos (36.5 mg, 0.08 mmol), Cs₂CO₃ (374.2 mg, 1.15 mmol) and Pd(OAc)₂ (8.6 mg, 0.04 mmol) at room temperature. The resulting mixture was stirred at 100° C. for 2 h under N₂. After the reaction was completed, the reaction mixture was diluted with water and extracted with ethyl acetate. The combined organic layers were washed with brine, dried over anhydrous Na₂SO₄ and filtered. The filtrate was concentrated under reduced pressure. The residue was purified by reverse phase flash column chromatography with MeOH/H₂O (80/20, v/v) and then purified by Prep-HPLC with the following conditions (Column: XSelect CSH Prep C18 OBD Column, 19×250 mm, 5 μm; Mobile Phase A: Water (10 mmol/L NH₄HCO₃), Mobile Phase B: MeOH-HPLC; Flow rate: 25 mL/min; Gradient: 77% B to 77% B in 12 min; Wave Length: 254 nm) to afford (E)-3-(2-(dimethylamino)ethylidene)-1-(4-((4-((6-ethylpyridin-3-yl)oxy)-3-methylphenyl)amino)pyrido[3,2-d]pyrimidin-6-yl)pyrrolidin-2-one (Compound 72) (9.4 mg, 97%) as a light yellow solid. LCMS (ESI, m/z): [M+H]⁺=510.3. ¹H NMR (400 MHz, DMSO-d₆): δ 9.54 (s, 1H), 8.97 (d, J=9.2 Hz, 1H), 8.60 (s, 1H), 8.26-8.22 (m, 2H), 7.87-7.84 (m, 2H), 7.28-7.24 (m, 2H), 7.02 (d, J=8.4 Hz, 1H), 6.58-6.52 (m, 1H), 4.36-4.32 (m, 2H), 3.11 (d, J=6.8 Hz, 2H), 2.88-2.84 (m, 2H), 2.76-2.70 (m, 2H), 2.24-2.21 (m, 9H), 1.24-1.21 (m, 3H).

### Example S67: Synthesis of (E)-3-(2-(dimethylamino)ethylidene)-1-(4-((4-((6-ethylpyridin-3-yl)oxy)-3-methylphenyl)amino)pyrido[3,4-d]pyrimidin-6-yl)pyrrolidin-2-one (Compound 73)

### Step 1. Synthesis of 6-chloro-N-(4-((6-ethylpyridin-3-yl)oxy)-3-methylphenyl)pyrido[3,4-d]pyrimidin-4-amine

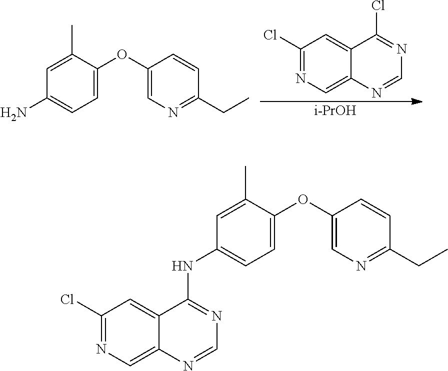 [SMILES: "i-PrOH" ; CCc1ccc(Oc2ccc(N)cc2C)cn1 ; Clc1cc2c(Cl)ncnc2cn1 ; CCc1ccc(Oc2ccc(Nc3ncnc4cnc(Cl)cc34)cc2C)cn1]

To a solution of 4-((6-ethylpyridin-3-yl)oxy)-3-methylaniline (300.0 mg, 1.31 mmol) in i-PrOH (10.0 mL) was added 4,6-dichloropyrido[3,4-d]pyrimidine (263.0 mg, 1.31 mmol) at room temperature. The resulting mixture was stirred at room temperature for 2 h. After the reaction was completed, the resulting mixture was concentrated under reduced pressure. The residue was purified by flash column chromatography with CH₂Cl₂/MeOH (10/1, v/v) to afford 6-chloro-N-(4-((6-ethylpyridin-3-yl)oxy)-3-methylphenyl)pyrido[3,4-d]pyrimidin-4-amine (370.0 mg, 68%) as a yellow solid. LCMS (ESI, m/z): [M+H]⁺=392.1.

### Step 2. Synthesis of (E)-3-(2-(dimethylamino)ethylidene)-1-(4-((4-((6-ethylpyridin-3-yl)oxy)-3-methylphenyl)amino)pyrido[3,4-d]pyrimidin-6-yl)pyrrolidin-2-one (Compound 73)

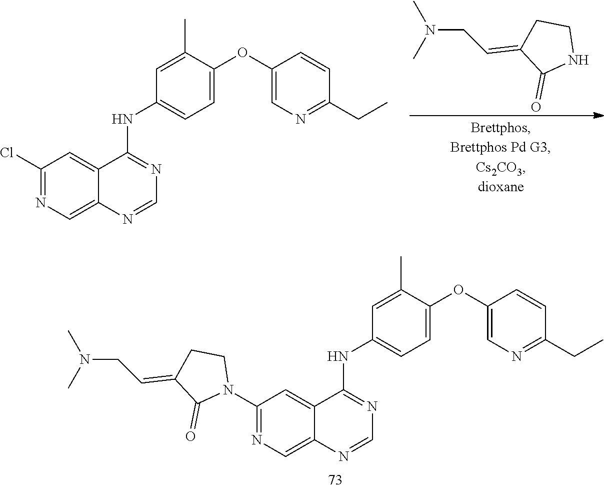

To a solution of 6-chloro-N-(4-((6-ethylpyridin-3-yl)oxy)-3-methylphenyl)pyrido[3,4-d]pyrimidin-4-amine (150.0 mg, 0.38 mmol) in dioxane (10.0 mL) were added (E)-3-(2-(dimethylamino)ethylidene)pyrrolidin-2-one (68.1 mg, 0.57 mmol), Cs₂CO₃ (374.2 mg, 1.15 mmol), Brettphos (41.1 mg, 0.08 mmol) and Brettphos Pd G3 (34.7 mg, 0.04 mmol) at room temperature under N₂, The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (90/10, v/v) and then purified by Prep-HPLC with the following conditions (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 35% B to 50% B in 8 min; Wave Length: 254 nm) to afford (E)-3-(2-(dimethylamino)ethylidene)-1-(4-((4-((6-ethylpyridin-3-yl)oxy)-3-methylphenyl)amino)pyrido[3,4-d]pyrimidin-6-yl)pyrrolidin-2-one (Compound 73) (53.3 mg, 26%) as a yellow solid. LCMS (ESI, m/z): [M+H]⁺=510.3. ¹H NMR (400 MHz, DMSO-d₆): δ 10.27 (s, 1H), 9.19 (s, 1H), 9.06 (s, 1H), 8.61 (s, 1H), 8.24-8.23 (m, 1H), 7.77 (s, 1H), 7.69 (d, J=8.4 Hz, 1H), 7.28-7.23 (m, 2H), 6.98 (d, J=8.8 Hz, 1H), 6.55-6.51 (m, 1H), 4.18-4.15 (m, 2H), 3.10 (d, J=6.4 Hz, 2H), 2.88-2.82 (m, 2H), 2.77-2.71 (m, 2H), 2.23-2.20 (m, 9H), 1.25-1.21 (m, 3H).

### Example S68: Synthesis of (3E)-3 [2-(dimethylamino)ethylidene]-1-{4-[(3-methyl-4-{[1,2,4]triazolo[1,5-a]pyridin-7-yloxy}phenyl)amino]pyrido[3,4-d]pyrimidin-6-yl)pyrrolidin-2-one (Compound 74)

### Step 1. Synthesis of (3E)-3-[2-(dimethylamino)ethylidene]-1-{4-[(3-methyl-4-{[1,2,4]triazolo[1,5-a]pyridin-7-yloxy}phenyl)amino]pyrido[3,4-d]pyrimidin-6-yl}pyrrolidin-2-one (Compound 74)

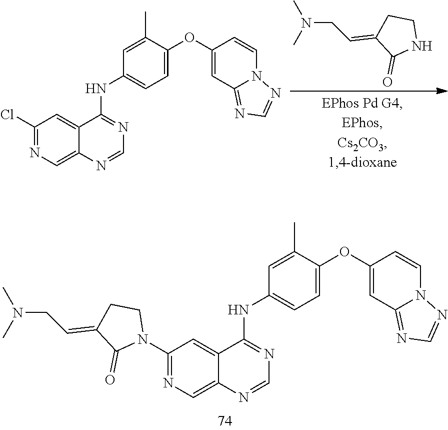

To a solution of 6-chloro-N-(3-methyl-4-{[1,2,4]triazolo[1,5-a]pyridin-7-yloxy}phenyl)pyrido[3,4-d]pyrimidin-4-amine (200.0 mg, 0.50 mmol) in dioxane (20.0 mL) was added (3E)-3-[2-(dimethylamino)ethylidene]pyrrolidin-2-one (76.4 mg, 0.50 mmol), Cs₂CO₃ (403.4 mg, 1.24 mmol), EPhos (53.2 mg, 0.10 mmol) and EPhos Pd G4 (44.9 mg, 0.05 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions Column: (XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 25% B to 40% B in 8 min; Wave Length: 254 nm) to afford (3E)-3-[2-(dimethylamino)ethylidene]-1-{4-[(3-methyl-4-{[1,2,4]triazolo[1,5-a]pyridin-7-yloxy}phenyl)amino]pyrido[3,4-d]pyrimidin-6-yl}pyrrolidin-2-one (Compound 74) (27.8 mg, 10%) as a white solid. LCMS (ESI, m/z): [M+H]⁺=522.2. ¹H NMR (400 MHz, DMSO-d₆): δ 10.35 (s, 1H), 9.22 (s, 1H), 9.10 (s, 1H), 8.95 (d, J=7.6 Hz, 1H), 8.65 (s, 1H), 8.39 (s, 1H), 7.87-7.82 (m, 2H), 7.24 (d, J=8.4 Hz, 1H), 7.06-7.03 (m, 1H), 6.83 (d, J=2.4 Hz, 1H), 6.56-6.53 (m, 1H), 4.20-4.16 (m, 2H), 3.11 (d, J=6.8 Hz, 2H), 2.89-2.85 (m, 2H), 2.22-2.21 (m, 9H).

### Example S69: Synthesis of (3E)-3-[2-(dimethylamino)ethylidene]-1-[4-({3-methyl-4-[(6-methylpyridin-3 yl)oxy]phenyl}amino)pyrido[3,4-d]pyrimidin-6-yl]pyrrolidin-2-one (Compound 75)

### Step 1. Synthesis of (3E)-3-[2-(dimethylamino)ethylidene]-1-[4-({3-methyl-4-[(6-methylpyridin-3-yl)oxy]phenyl}amino)pyrido[3,4-d]pyrimidin-6-yl]pyrrolidin-2-one (Compound 75)

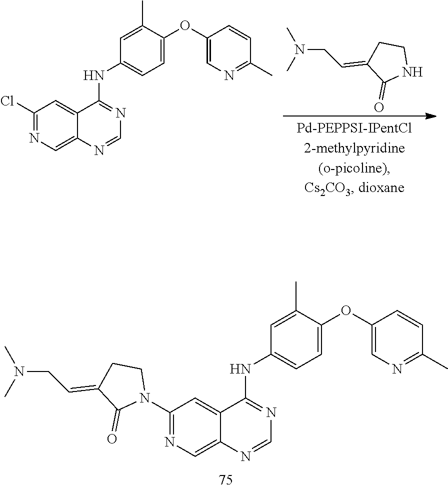

To a stirred solution of 6-chloro-N-{3-methyl-4-[(6-methylpyridin-3-yl)oxy]phenyl}pyrido[3,4-d]pyrimidin-4-amine (200.0 mg, 0.52 mmol) in dioxane (7.0 mL) was added (3E)-3-[2-(dimethylamino)ethylidene]pyrrolidin-2-one (97.9 mg, 0.63 mmol), Pd-PEPPSI-IPentCl 2-methylpyridine (o-picoline) (44.4 mg, 0.05 mmol) and Cs₂CO₃ (517.4 mg, 1.58 mmol) at room temperature under N₂. The resulting mixture was stirred at 100° C. for 2 h. After the reaction was completed, the resulting mixture was filtered. The filtrate was concentrated under vacuum. The residue was purified by flash chromatography with CH₂Cl₂/MeOH (85/15, v/v) and then purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 34% B to 34% B in 15 min; Wave Length: 254 nm) to afford (3E)-3-[2-(dimethylamino)ethylidene]-1-[4-({3-methyl-4-[(6-methylpyridin-3-yl)oxy]phenyl}amino)pyrido[3,4-d]pyrimidin-6-yl]pyrrolidin-2-one (Compound 75) (11.7 mg, 4%) as a yellow solid. LCMS (ESI, m/z): [M+H]⁺=496.2. ¹H NMR (400 MHz, DMSO-d₆): δ 10.27 (s, 1H), 9.19 (s, 1H), 9.08 (s, 1H), 8.61 (s, 1H), 8.21-8.20 (m, 1H), 7.77 (d, J=2.0 Hz, 1H), 7.71-7.68 (m, 1H), 7.25-7.21 (m, 2H), 6.97 (d, J=8.4 Hz, 1H), 6.54-6.51 (m, 1H), 4.19-4.15 (m, 2H), 3.20 (d, J=6.8 Hz, 2H), 2.89-2.84 (m, 2H), 2.45 (s, 3H), 2.23-2.21 (m, 9H).

### Example S70: Synthesis of (3E)-3 [2-(dimethylamino)ethylidene]-1-[4-({2-fluoro-5-methyl-4-[(1-methyl-1,3-benzodiazol-5 yl)oxy]phenyl}amino)pyrido[3,4-d]pyrimidin-6-yl]pyrrolidin-2-one (Compound 76)

### Step 1. Synthesis of 6-chloro-N-{2-fluoro-5-methyl-4-[(1-methyl-1,3-benzodiazol-5-yl)oxy]phenyl}pyrido[3,4-d]pyrimidin-4-amine

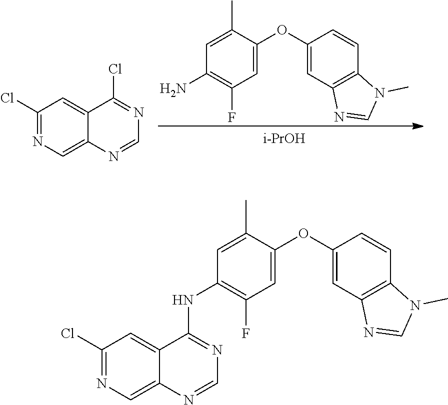 [SMILES: "i-PrOH" ; Clc1cc2c(Cl)ncnc2cn1 ; Cc1cc(N)c(F)cc1Oc1ccc2c(c1)ncn2C ; Cc1cc(Nc2ncnc3cnc(Cl)cc23)c(F)cc1Oc1ccc2c(c1)ncn2C]

To a solution of 4,6-dichloropyrido[3,4-d]pyrimidine (200.0 mg, 1.00 mmol) in i-PrOH (5.0 mL) was added 2-fluoro-5-methyl-4-[(1-methyl-1,3-benzodiazol-5-yl)oxy]aniline (271.1 mg, 1.00 mmol) at room temperature. The resulting mixture was stirred at room temperature for 4 h. After the reaction was completed, the resulting mixture was concentrated under reduced pressure. The residue was purified by flash column chromatography with CH₂Cl₂/MeOH (10/1, v/v) to afford 6-chloro-N-{2-fluoro-5-methyl-4-[(1-methyl-1,3-benzodiazol-5-yl)oxy]phenyl}pyrido[3,4-d]pyrimidin-4-amine (150.0 mg, 34%) as a brown solid. LCMS (ESI, m/z): [M+H]⁺=435.1.

### Step 2. Synthesis of (3E)-3-[2-(dimethylamino)ethylidene]-1-[4-({2-fluoro-5-methyl-4-[(1-methyl-1,3-benzodiazol-5-yl)oxy]phenyl}amino)pyrido[3,4-d]pyrimidin-6-yl]pyrrolidin-2-one (Compound 76)

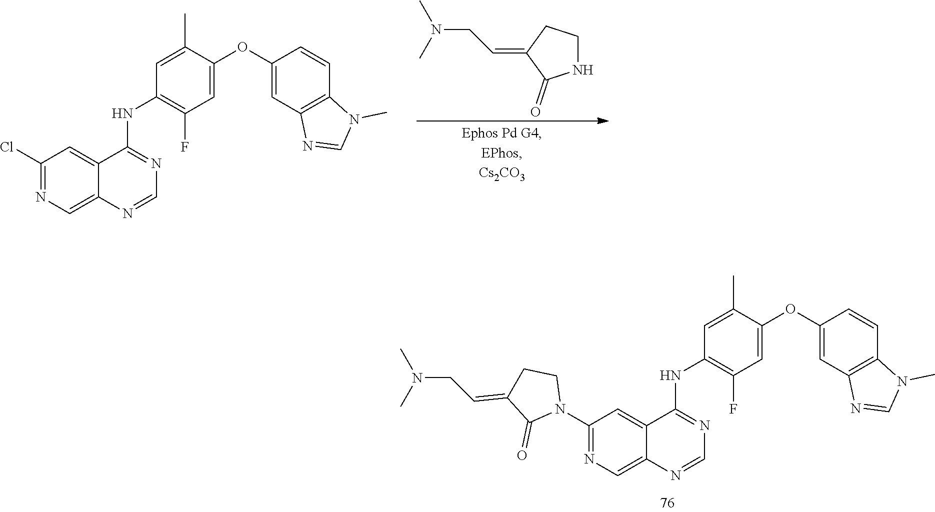

To a solution of 6-chloro-N-{2-fluoro-5-methyl-4-[(1-methyl-1,3-benzodiazol-5-yl)oxy]phenyl}pyrido[3,4-d]pyrimidin-4-amine (100.0 mg, 0.23 mmol) in 1,4-dioxane (2.0 mL) were added (3E)-3-[2-(dimethylamino)ethylidene]pyrrolidin-2-one (35.5 mg, 0.23 mmol), Cs₂CO₃ (149.8 mg, 0.46 mmol), EPhos Pd G4 (21.1 mg, 0.02 mmol) and EPhos (24.6 mg, 0.05 mmol) at room temperature under N₂. The mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) and then purified by Prep-HPLC with the following conditions: (Column: Xselect CSH C18 OBD Column 30×150 mm, 5 μm; Mobile Phase A: Water (10 mmol/L NH₄HCO₃), Mobile Phase B: ACN; Flow rate: 60 mL/min; Gradient: 30% B to 40% B in 12 min; Wave Length: 220 nm) to afford (3E)-3-[2-(dimethylamino)ethylidene]-1-[4-({2-fluoro-5-methyl-4-[(1-methyl-1,3-benzodiazol-5-yl)oxy]phenyl}amino)pyrido[3,4-d]pyrimidin-6-yl]pyrrolidin-2-one (14.0 mg, 10%) as a light yellow solid. LCMS (ESI, m/z): [M+H]⁺=553.3. ¹H NMR (400 MHz, DMSO-d₆): δ 10.18 (s, 1H), 9.07-9.02 (m, 2H), 8.50 (s, 1H), 8.14 (s, 1H), 7.50-7.43 (m, 2H), 7.22 (d, J=2.0 Hz, 1H), 7.01-6.98 (m, 1H), 6.64 (d, J=10.8 Hz, 1H), 6.52-6.49 (m, 1H), 4.15-4.12 (m, 2H), 3.79 (s, 3H), 3.08 (d, J=6.8 Hz, 2H), 2.86-2.82 (m, 2H), 2.25 (s, 3H), 2.19 (s, 6H).

### Example S71: Synthesis of (3E)-3 [2-(dimethylamino)ethylidene]-1-[4-({2-fluoro-3-methyl-4-[(1-methyl-1,3-benzodiazol-5 yl)oxy]phenyl}amino)pyrido[3,4-d]pyrimidin-6-yl]pyrrolidin-2-one (Compound 77)

### Step 1. Synthesis of 5-(3-fluoro-2-methyl-4-nitrophenoxy)-1-methyl-1,3-benzodiazole

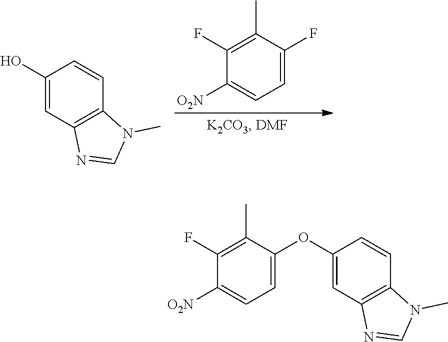

To a stirred mixture of 1-methyl-1,3-benzodiazol-5-ol (3.0 g, 20.25 mmol) and 1,3-difluoro-2-methyl-4-nitrobenzene (3.5 g, 20.25 mmol) in DMF (50.0 mL) was added K₂CO₃ (8.4 g, 60.74 mmol) at room temperature. The resulting mixture was stirred at room temperature for 3 h. After the reaction was competed, the resulting mixture was diluted with H₂O and extracted with EtOAc. The combined organic layers were washed with brine, dried over anhydrous Na₂SO₄ and filtered. The filtrate was concentrated under reduced pressure. The residue was purified by reverse phase flash column chromatography with H₂O/CH₃CN (50/50, v/v) to afford 5-(3-fluoro-2-methyl-4-nitrophenoxy)-1-methyl-1,3-benzodiazole (600.0 mg, 9%) as a light yellow solid. LCMS (ESI, m/z): [M+H]⁺=302.1.

### Step 2. Synthesis of 2-fluoro-3-methyl-4-[(1-methyl-1,3-benzodiazol-5-yl)oxy]aniline

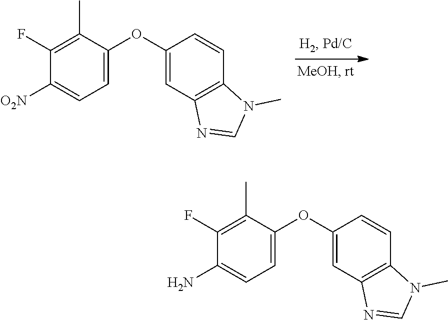

To a stirred mixture of 5-(3-fluoro-2-methyl-4-nitrophenoxy)-1-methyl-1,3-benzodiazole (580.0 mg, 1.93 mmol) in MeOH (10.0 mL) was added Pd/C (202.8 mg, 10%) at room temperature under N₂. The resulting mixture was stirred at room temperature for 1 h under H₂. After the reaction was completed, the resulting mixture was filtered. The filtrate was concentrated under reduced pressure. The residue was purified by flash column chromatography with CH₂Cl₂/MeOH (8/1, v/v) to afford 2-fluoro-3-methyl-4-[(1-methyl-1,3-benzodiazol-5-yl)oxy]aniline (500.0 mg, 95%) as a yellow solid. LCMS (ESI, m/z): [M+H]⁺=272.1.

### Step 3. Synthesis of 6-chloro-N-{2-fluoro-3-methyl-4-[(1-methyl-1,3-benzodiazol-5-yl)oxy]phenyl}pyrido[3,4-d]pyrimidin-4-amine

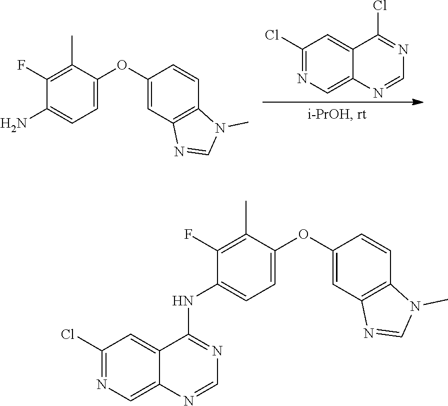 [SMILES: Cc1c(Oc2ccc3c(c2)ncn3C)ccc(N)c1F ; Clc1cc2c(Cl)ncnc2cn1 ; Cc1c(Oc2ccc3c(c2)ncn3C)ccc(Nc2ncnc3cnc(Cl)cc23)c1F]

To a stirred mixture of 2-fluoro-3-methyl-4-[(1-methyl-1,3-benzodiazol-5-yl)oxy]aniline (450.0 mg, 1.66 mmol) in i-PrOH (10.0 mL) was added 4,6-dichloropyrido[3,4-d]pyrimidine (331.8 mg, 1.66 mmol) at room temperature under N₂. The resulting mixture was stirred at room temperature for 2 h. After the reaction was completed, the resulting mixture was concentrated under reduced pressure. The residue was purified by flash column chromatography with CH₂Cl₂/MeOH (5/1, v/v) to afford 6-chloro-N-{2-fluoro-3-methyl-4-[(1-methyl-1,3-benzodiazol-5-yl)oxy]phenyl}pyrido[3,4-d]pyrimidin-4-amine (600.0 mg, 83%) as a yellow solid. LCMS (ESI, m/z): [M+H]⁺=435.1.

### Step 4. Synthesis of (3E)-3-[2-(dimethylamino)ethylidene]-1-[4-({2-fluoro-3-methyl-4-[(1-methyl-1,3-benzodiazol-5-yl)oxy]phenyl}amino)pyrido[3,4-d]pyrimidin-6-yl]pyrrolidin-2-one (Compound 77)

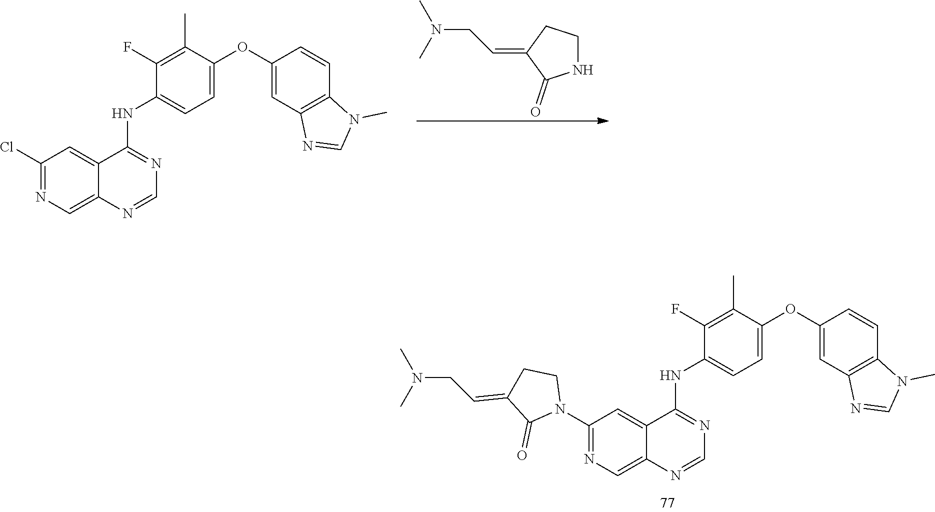

To a stirred mixture of 6-chloro-N-{2-fluoro-3-methyl-4-[(1-methyl-1,3-benzodiazol-5-yl)oxy]phenyl}pyrido[3,4-d]pyrimidin-4-amine (100.0 mg, 0.23 mmol) and (3E)-3-[2-(dimethylamino)ethylidene]pyrrolidin-2-one (53.2 mg, 0.35 mmol) in dioxane (6.0 mL) were added Cs₂CO₃ (224.8 mg, 0.69 mmol), EPhos (49.2 mg, 0.09 mmol) and EPhos Pd G4 (42.3 mg, 0.05 mmol) at room temperature. The resulting mixture was stirred at 100° C. for 16 h under N₂. After the reaction was completed, the resulting mixture was diluted with H₂O and extracted with ethyl acetate. The combined organic layers were washed with brine, dried over anhydrous Na₂SO₄ and filtered. The filtrate was concentrated under reduced pressure. The residue was purified by flash column chromatography with CH₂Cl₂/MeOH (5/1, v/v) and then purified by Prep-HPLC with the following conditions: (Column: Torus 2-PIC, 01083900811201; Mobile Phase A: CO₂, Mobile Phase B: MeOH (0.5% 2M NH₃-MeOH)-HPLC; Flow rate: 60 mL/min; Gradient: isocratic 50% B; Wave Length: 254 nm) to afford (3E)-3-[2-(dimethylamino)ethylidene]-1-[4-({2-fluoro-3-methyl-4-[(1-methyl-1,3-benzodiazol-5-yl)oxy]phenyl}amino)pyrido[3,4-d]pyrimidin-6-yl]pyrrolidin-2-one (Compound 77) (7.0 mg, 5%) as a white solid. LCMS (ESI, m/z): [M+H]⁺=553.4. ¹H NMR (400 MHz, DMSO-d₆): δ 10.33 (s, 1H), 9.18 (s, 1H), 9.09 (s, 1H), 8.54 (s, 1H), 8.23 (s, 1H), 7.64 (d, J=8.8 Hz, 1H), 7.28-7.25 (m, 2H), 7.10-7.08 (m, 1H), 6.63 (d, J=8.8 Hz, 1H), 6.54-6.50 (s, 1H), 4.19-4.16 (m, 2H), 3.87 (s, 3H), 3.16 (d, J=6.4 Hz, 2H), 2.89-2.84 (m, 2H), 2.33-2.25 (m, 9H).

### Example S72: Synthesis of (3E)-3 [2-(dimethylamino)ethylidene]-1-{4-[(2 fluoro-3-methyl-4-{[1,2,4]triazolo[1,5-a]pyridin-7-yloxy}phenyl)amino]pyrido[3,2-d]pyrimidin-6-yl}pyrrolidin-2-one hydrochloride (Compound 78)

### Step 1. Synthesis of 6-chloro-N-(2-fluoro-3-methyl-4-{[1,2,4]triazolo[1,5-a]pyridin-7-yloxy}phenyl)pyrido[3,2-d]pyrimidin-4-amine

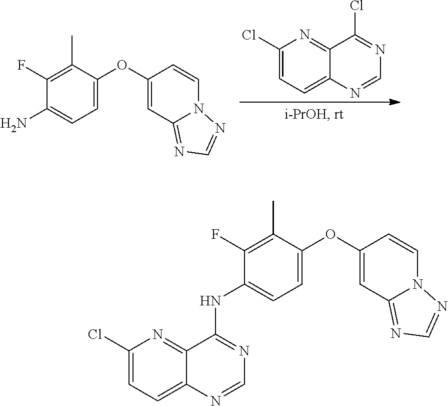 [SMILES: Cc1c(Oc2ccn3ncnc3c2)ccc(N)c1F ; Clc1ccc2ncnc(Cl)c2n1 ; Cc1c(Oc2ccn3ncnc3c2)ccc(Nc2ncnc3ccc(Cl)nc23)c1F]

A mixture of 2-fluoro-3-methyl-4-{[1,2,4]triazolo[1,5-a]pyridin-7-yloxy}aniline (90.0 mg, 0.34 mmol) and 4,6-dichloropyrido[3,2-d]pyrimidine (69.7 mg, 0.34 mmol) in isopropyl alcohol (8.0 mL) was stirred at room temperature for 16 h. After the reaction was completed, the resulting mixture was concentrated under reduced pressure. The residue was purified by flash column chromatography with CH₂Cl₂/CH₃OH (10/1, v/v) to afford 6-chloro-N-(2-fluoro-3-methyl-4-{[1,2,4]triazolo[1,5-a]pyridin-7-yloxy}phenyl)pyrido[3,2-d]pyrimidin-4-amine (100.0 mg, 68%) as a yellow solid. LCMS (ESI, m/z): [M+H]⁺=422.1.

### Step 2. Synthesis of (3E)-3-[2-(dimethylamino)ethylidene]-1-{4-[(2-fluoro-3-methyl-4-{[1,2,4]triazolo[1,5-a]pyridin-7-yloxy}phenyl)amino]pyrido[3,2-d]pyrimidin-6-yl}pyrrolidin-2-one hydrochloride (Compound 78)

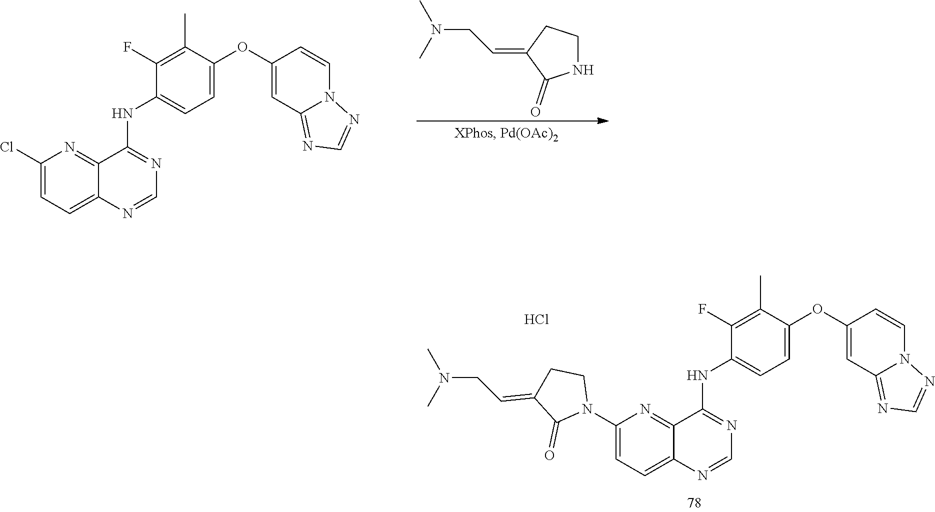 [SMILES: Cc1c(Oc2ccn3ncnc3c2)ccc(Nc2ncnc3ccc(Cl)nc23)c1F ; CN(C)C/C=C1\CCNC1=O ; Cc1c(Oc2ccn3ncnc3c2)ccc(Nc2ncnc3ccc(N4CC/C(=C\CN(C)C)C4=O)nc23)c1F ; Cl]

To a stirred mixture of 6-chloro-N-(2-fluoro-3-methyl-4-{[1,2,4]triazolo[1,5-a]pyridin-7-yloxy}phenyl)pyrido[3,2-d]pyrimidin-4-amine (80.0 mg, 0.19 mmol) and (3E)-3-[2-(dimethylamino)ethylidene]pyrrolidin-2-one (29.2 mg, 0.19 mmol) in 1,4-dioxane (3.0 mL) was added Cs₂CO₃ (185.4 mg, 0.57 mmol), XPhos (18.1 mg, 0.038 mmol) and Pd(OAc)₂ (4.3 mg, 0.02 mmol) at room temperature. The resulting mixture was stirred at 100° C. for 2 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) and then purified by Prep-HPLC with the following conditions Column: (XSelect CSH Fluoro Phenyl, 30×150 mm, 5 μm; Mobile Phase A: ACN, Mobile Phase B: Water (0.05% HCl); Flow rate: 60 mL/min; Gradient: 13% B to 18% B in 8 min, 18% B to 18% B in 14 min; Wave Length: 254 nm) to afford (3E)-3-[2-(dimethylamino)ethylidene]-1-{4-[(2-fluoro-3-methyl-4-{[1,2,4]triazolo[1,5-a]pyridin-7-yloxy}phenyl)amino]pyrido[3,2-d]pyrimidin-6-yl}pyrrolidin-2-one hydrochloride (Compound 78) (9.0 mg, 8%) as a yellow solid. LCMS (ESI, m/z): [M+H]⁺=540.1. ¹H NMR (400 MHz, DMSO-d₆): δ 9.90 (s, 1H), 9.73 (s, 1H), 9.03-8.98 (m, 2H), 8.66 (s, 1H), 8.43 (s, 1H), 8.35 (d, J=9.2 Hz, 1H), 8.01-7.96 (m, 1H), 7.19 (d, J=8.8 Hz, 1H), 7.10-7.08 (m, 1H), 6.95 (s, 1H), 6.57-6.54 (m, 1H), 4.35-4.32 (m, 2H), 4.02-3.96 (m, 2H), 3.04-3.00 (m, 2H), 2.86 (s, 6H), 2.19 (s, 3H).

### Example S73: Synthesis of (3E)-3 [2-(dimethylamino)ethylidene]-1-{4-[(2 fluoro-5-methyl-4-{[1,2,4]triazolo[1,5-a]pyridin-7-yloxy}phenyl)amino]pyrido[3,2-d]pyrimidin-6-yl}pyrrolidin-2-one (Compound 79)

### Step 1. Synthesis of (3E)-3-[2-(dimethylamino)ethylidene]-1-{4-[(2-fluoro-5-methyl-4-{[1,2,4]triazolo[1,5-a]pyridin-7-yloxy}phenyl)amino]pyrido[3,2-d]pyrimidin-6-yl}pyrrolidin-2-one (Compound 79)

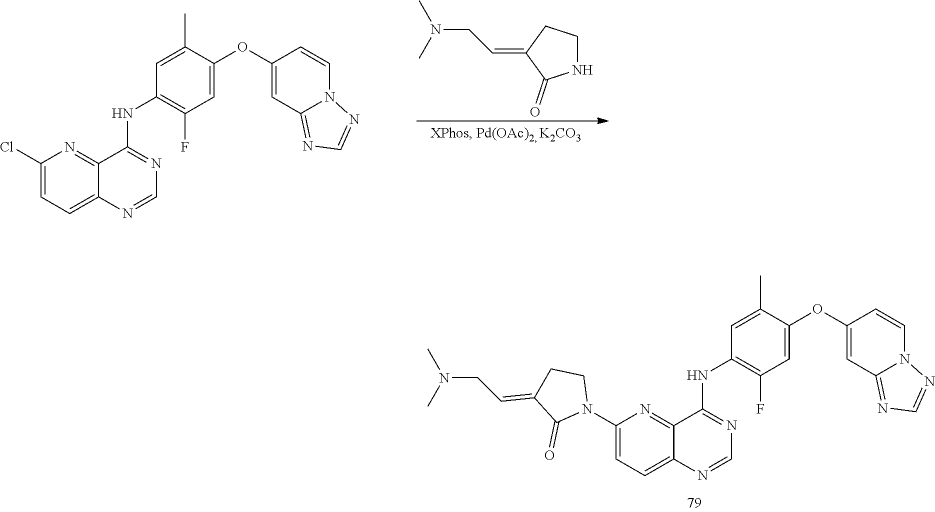 [SMILES: Cc1cc(Nc2ncnc3ccc(Cl)nc23)c(F)cc1Oc1ccn2ncnc2c1 ; CN(C)C/C=C1\CCNC1=O ; Cc1cc(Nc2ncnc3ccc(N4CC/C(=C\CN(C)C)C4=O)nc23)c(F)cc1Oc1ccn2ncnc2c1]

To a stirred solution 6-chloro-N-(2-fluoro-5-methyl-4-{[1,2,4]triazolo[1,5-a]pyridin-7-yloxy}phenyl)pyrido[3,2-d]pyrimidin-4-amine (180.0 mg, 0.43 mmol) in dioxane (10.0 mL) was added (3E)-3-[2-(dimethylamino)ethylidene]pyrrolidin-2-one (65.8 mg, 0.43 mmol), K₂CO₃ (278.1 mg, 0.85 mmol), XPhos (49.4 mg, 0.08 mmol), Pd(OAc)₂ (24.5 mg, 0.04 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 33% B to 38% B in 8 min; Wave Length: 254 nm) to afford (3E)-3-[2-(dimethylamino)ethylidene]-1-{4-[(2-fluoro-5-methyl-4-{[1,2,4]triazolo[1,5-a]pyridin-7-yloxy}phenyl)amino]pyrido[3,2-d]pyrimidin-6-yl}pyrrolidin-2-one (Compound 79) (7.4 mg, 3%) as a white solid. LCMS (ESI, m/z): [M+H]⁺=540.3. ¹H NMR (400 MHz, DMSO-d₆): δ 9.58 (s, 1H), 9.02-8.97 (m, 2H), 8.61 (s, 1H), 8.42 (s, 1H), 8.29 (d, J=9.2 Hz, 1H), 8.01 (d, J=8.0 Hz, 1H), 7.35 (d, J=10.8 Hz, 1H), 7.08-7.05 (m, 1H), 6.94 (d, J=2.4 Hz, 1H), 6.61-6.55 (m, 1H), 4.30-4.26 (m, 2H), 3.11 (d, J=6.8 Hz, 2H), 2.90-2.86 (m, 2H), 2.21 (s, 9H).

### Example S74: Synthesis of (3E)-3 [2-(dimethylamino)ethylidene]-1-(4-{[3-methyl-4-(pyridin-3-yloxy)phenyl]amino}pyrido[3,4-d]pyrimidin-6-yl)pyrrolidin-2-one (Compound 80)

### Step 1. Synthesis of 3-(2-methyl-4-nitrophenoxy)pyridine

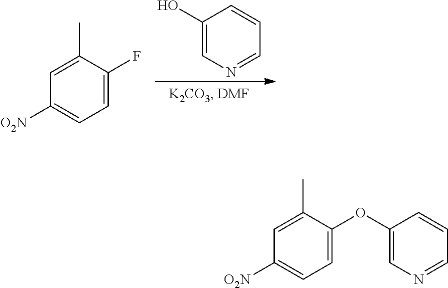 [SMILES: Cc1cc([N+](=O)[O-])ccc1F ; Oc1cccnc1 ; Cc1cc([N+](=O)[O-])ccc1Oc1cccnc1]

To a solution of 1-fluoro-2-methyl-4-nitrobenzene (5.0 g, 32.23 mmol) in DMF (200.0 mL) was added 3-hydroxypyridine (3.1 g, 32.23 mmol) and K₂CO₃ (10.9 g, 96.69 mmol) at room temperature. The resulting mixture was stirred at 70° C. for 16 h. After the reaction was completed, the mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (95/5, v/v) to afford 3-(2-methyl-4-nitrophenoxy)pyridine (3.0 g, 36%) as a yellow solid. LCMS (ESI, m/z): [M+H]⁺=231.1.

### Step 2. Synthesis of 3-methyl-4-(pyridin-3-yloxy)aniline

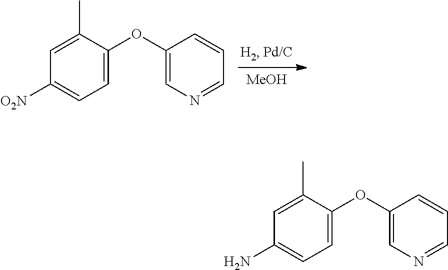 [SMILES: "MeOH" ; Cc1cc([N+](=O)[O-])ccc1Oc1cccnc1 ; Cc1cc(N)ccc1Oc1cccnc1]

To a solution of 3-(2-methyl-4-nitrophenoxy)pyridine (500.0 mg, 2.17 mmol) in methanol (15.0 mL) was added Pd/C (150.0 mg, 10%) at room temperature under N₂. The resulting mixture was stirred at room temperature for 16 h under H₂. After the reaction was completed, the mixture was filtered. The filtrate was concentrated under vacuum to afford 3-methyl-4-(pyridin-3-yloxy)aniline (400.0 mg, crude) as a yellow solid. LCMS (ESI, m/z): [M+H]⁺=201.1.

### Step 3. Synthesis of 6-chloro-N-[3-methyl-4-(pyridin-3-yloxy)phenyl]pyrido[3,4-d]pyrimidin-4-amine

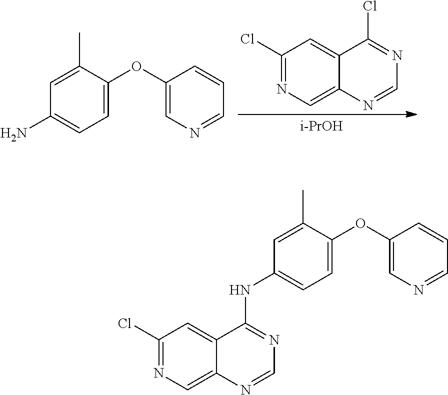 [SMILES: "i-PrOH" ; Cc1cc(N)ccc1Oc1cccnc1 ; Clc1cc2c(Cl)ncnc2cn1 ; Cc1cc(Nc2ncnc3cnc(Cl)cc23)ccc1Oc1cccnc1]

To a solution of 3-methyl-4-(pyridin-3-yloxy)aniline (500.0 mg, crude) in i-PrOH (10.0 mL) was added 4,6-dichloropyrido[3,4-d]pyrimidine (500.0 mg, 2.50 mmol) at room temperature. The resulting mixture was stirred at room temperature for 16 h. After the reaction was completed, the resulting mixture was concentrated under vacuum. The residue was purified by flash column chromatography with CH₂Cl₂/MeOH (85/15, v/v) to afford 6-chloro-N-[3-methyl-4-(pyridin-3-yloxy)phenyl]pyrido[3,4-d]pyrimidin-4-amine (500.0 mg, 50%) as a yellow solid. LCMS (ESI, m/z): [M+H]⁺=364.1.

### Step 4. Synthesis of (3E)-3-[2-(dimethylamino)ethylidene]-1-(4-{[3-methyl-4-(pyridin-3-yloxy)phenyl]amino}pyrido[3,4-d]pyrimidin-6-yl)pyrrolidin-2-one (Compound 80)

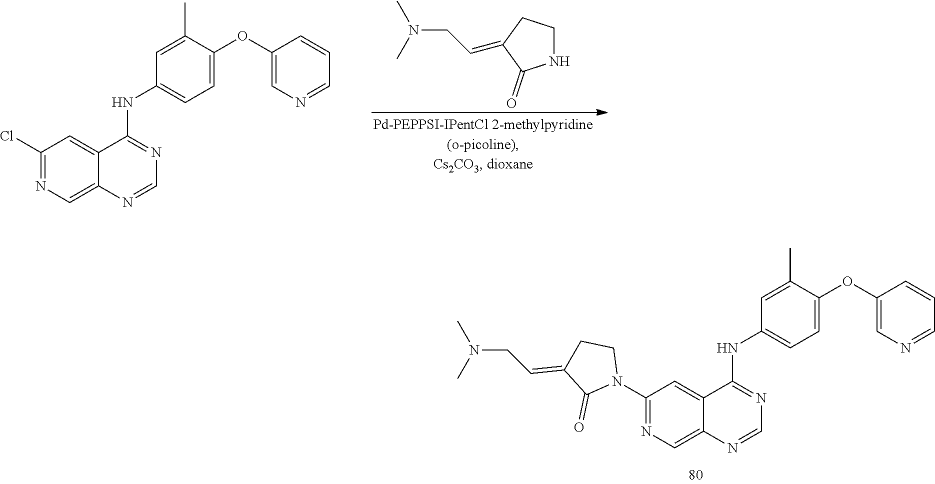 [SMILES: Cc1cc(Nc2ncnc3cnc(Cl)cc23)ccc1Oc1cccnc1 ; CN(C)C/C=C1\CCNC1=O ; Cc1cc(Nc2ncnc3cnc(N4CC/C(=C\CN(C)C)C4=O)cc23)ccc1Oc1cccnc1]

To a solution of 6-chloro-N-[3-methyl-4-(pyridin-3-yloxy)phenyl]pyrido[3,4-d]pyrimidin-4-amine (150.0 mg, 0.41 mmol) in dioxane (5.0 mL) was added Cs₂CO₃ (403.0 mg, 1.24 mmol), (3E)-3-[2-(dimethylamino)ethylidene]pyrrolidin-2-one (63.6 mg, 0.41 mmol) and Pd-PEPPSI-IPentCl 2-methylpyridine (o-picoline) (34.7 mg, 0.04 mmol) at room temperature under N₂. The resulting mixture was stirred at 100° C. for 2 h. After the reaction was completed, the resulting mixture was cooled to room temperature and filtered. The filtrate was concentrated under vacuum. The residue was purified by flash chromatography with CH₂Cl₂/MeOH (85/15, v/v) and then purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 30% B to 40% B in 8 min; Wave Length: 254 nm) to afford (3E)-3-[2-(dimethylamino)ethylidene]-1-(4-{[3-methyl-4-(pyridin-3-yloxy)phenyl]amino}pyrido[3,4-d]pyrimidin-6-yl)pyrrolidin-2-one (Compound 80) (2.3 mg, 1%) as a white solid. LCMS (ESI, m/z): [M+H]⁺=482.1. ¹H NMR (400 MHz, DMSO-d₆): δ 10.30 (s, 1H), 9.20 (s, 1H), 9.08 (s, 1H), 8.62 (s, 1H), 8.36-8.32 (m, 2H), 7.80 (s, 1H), 7.71-7.60 (m, 1H), 7.40 (d, J=4.4 Hz, 1H), 7.31-7.29 (m, 1H), 7.05 (d, J=8.8 Hz, 1H), 6.52-6.45 (m, 1H), 4.19-4.15 (m, 2H), 3.11-3.09 (m, 2H), 2.89-2.83 (m, 2H), 2.22-2.00 (m, 9H).

### Example S75: Synthesis of (E)-3-(2-(dimethylamino)ethylidene)-1-(4-((3-methyl-4-((3-methylimidazo[1,2-a]pyridin-7 yl)oxy)phenyl)amino)pyrido[3,2-d]pyrimidin-6-yl)pyrrolidin-2-one (Compound 81)

### Step 1. Synthesis of 6-chloro-N-(3-methyl-4-((3-methylimidazo[1,2-a]pyridin-7-yl)oxy)phenyl)pyrido[3,2-d]pyrimidin-4-amine

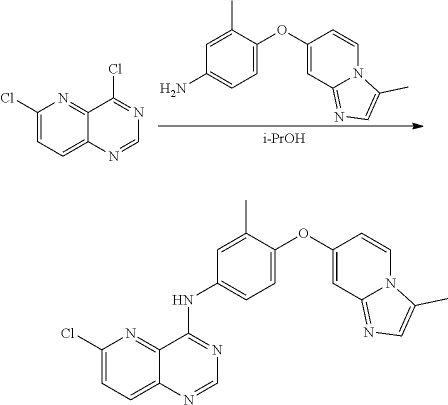 [SMILES: "i-PrOH" ; Clc1ccc2ncnc(Cl)c2n1 ; Cc1cc(N)ccc1Oc1ccn2c(C)cnc2c1 ; Cc1cc(Nc2ncnc3ccc(Cl)nc23)ccc1Oc1ccn2c(C)cnc2c1]

To a solution of 4,6-dichloropyrido[3,2-d]pyrimidine (130.0 mg, 0.65 mmol) in i-PrOH (15.0 mL) was added 3-methyl-4-((3-methylimidazo[1,2-a]pyridin-7-yl)oxy)aniline (170.0 mg, 0.65 mmol) at room temperature. The mixture was stirred at room temperature for 16 h. After the reaction was completed, the mixture was concentrated under reduced pressure. The residue was purified by flash chromatography with dichloromethane/methanol (90/10, v/v) to afford 6-chloro-N-(3-methyl-4-((3-methylimidazo[1,2-a]pyridin-7-yl)oxy)phenyl)pyrido[3,2-d]pyrimidin-4-amine (260.0 mg, 95%) as a yellow solid. LCMS (ESI, m/z): [M+H]⁺=417.0.

### Step 2. Synthesis of (E)-3-(2-(dimethylamino)ethylidene)-1-(4-((3-methyl-4-((3-methylimidazo[1,2-a]pyridin-7-yl)oxy)phenyl)amino)pyrido[3,2-d]pyrimidin-6-yl)pyrrolidin-2-one (Compound 81)

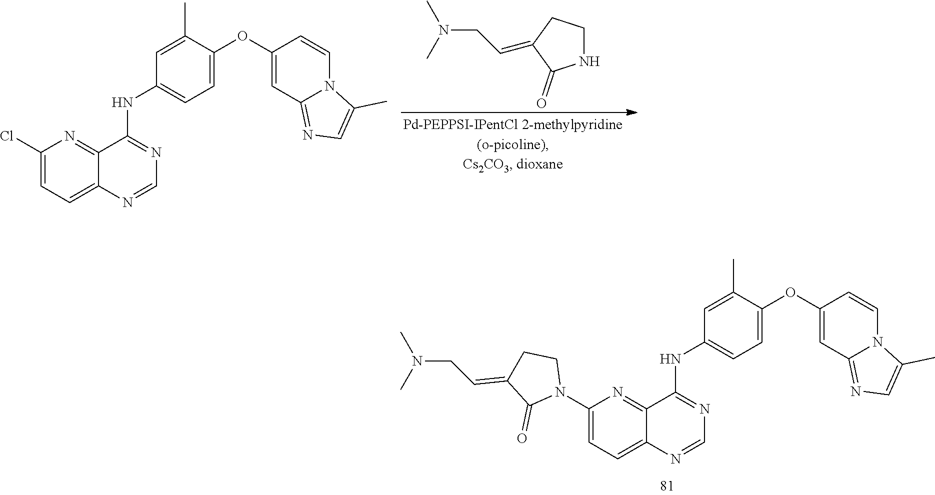

To a solution of 6-chloro-N-(3-methyl-4-((3-methylimidazo[1,2-a]pyridin-7-yl)oxy)phenyl)pyrido[3,2-d]pyrimidin-4-amine (200 mg, 0.48 mmol) in 1,4-dioxane (10.0 mL) was added Pd-PEPPSI-IPentCl 2-methylpyridine (o-picoline) (80.71 mg, 0.10 mmol), Cs₂CO₃ (470.2 mg, 1.44 mmol) and (E)-3-(2-(dimethylamino)ethylidene)pyrrolidin-2-one (73.9 mg, 0.48 mmol) at room temperature under N₂. The resulting mixture was stirred at 100° C. for 2 h. After the reaction was completed, the resulting mixture was filtered. The filtrate was concentrated under vacuum. The residue was purified by flash chromatography with CH₂Cl₂/MeOH (85/15, v/v) and then purified by Prep-HPLC with the following conditions (Column: XBridge Prep Phenyl OBD Column, 19×250 mm, 5 μm; Mobile Phase A: Water (10 mmol/L NH₄HCO₃), Mobile Phase B: ACN; Flow rate: 25 mL/min; Gradient: 45% B to 52% B in 12 min; Wave Length: 254 nm) to afford (E)-3-(2-(dimethylamino)ethylidene)-1-(4-((3-methyl-4-((3-methylimidazo[1,2-a]pyridin-7-yl)oxy)phenyl)amino)pyrido[3,2-d]pyrimidin-6-yl)pyrrolidin-2-one (Compound 81) (8.7 mg, 3%) as a white solid. LCMS (ESI, m/z): [M+H]⁺=535.3. ¹H NMR (400 MHz, DMSO-d₆): δ 9.58 (s, 1H), 8.98 (d, J=9.2 Hz, 1H), 8.63 (s, 1H), 8.28-8.25 (m, 2H), 7.92 (s, 2H), 7.23 (s, 1H), 7.18-7.16 (m, 1H), 6.85-6.82 (m, 1H), 6.56-6.53 (m, 2H), 4.37-4.34 (m, 2H), 3.13-3.11 (m, 2H), 2.89-2.85 (m, 2H), 2.44 (s, 3H), 2.23-2.21 (m, 9H).

### Example S76: Synthesis of (3E)-3 [2-(dimethylamino)ethylidene]-1-(4-{[3-methyl-4-([3-methylimidazo[1,2-a]pyridin-7 yl]oxy)phenyl]amino}pyrido[3,4-d]pyrimidin-6-yl)pyrrolidin-2-one (Compound 82)

### Step 1. Synthesis of tert-butyl N-[4-(benzyloxy)pyridin-2-yl]carbamate

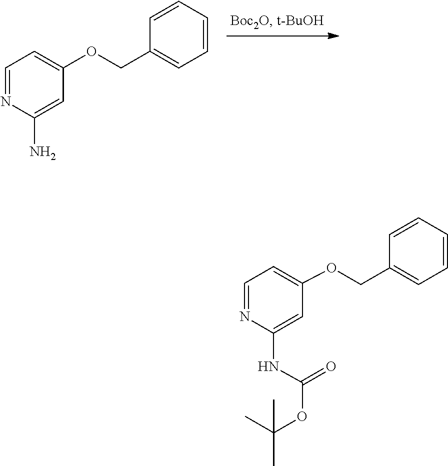 [SMILES: Nc1cc(OCc2ccccc2)ccn1 ; CC(C)(C)OC(=O)Nc1cc(OCc2ccccc2)ccn1]

To a solution of 4-(benzyloxy)pyridin-2-amine (2.0 g, 9.99 mmol) in t-BuOH (10.0 mL) was added Boc₂O (2397.8 mg, 10.98 mmol) at room temperature. The resulting solution was stirred at 50° C. for 1 h. After the reaction was completed, the mixture was diluted with EtOH and then filtered. The precipitated solids were collected to afford tert-butyl N-[4-(benzyloxy)pyridin-2-yl]carbamate (2.7 g, crude) as a white solid. LCMS (ESI, m/z): [M+H]⁺=301.1.

### Step 2. Synthesis of tert-butyl N-[4-(benzyloxy)pyridin-2-yl]-N-(prop-2-yn-1-yl)carbamate

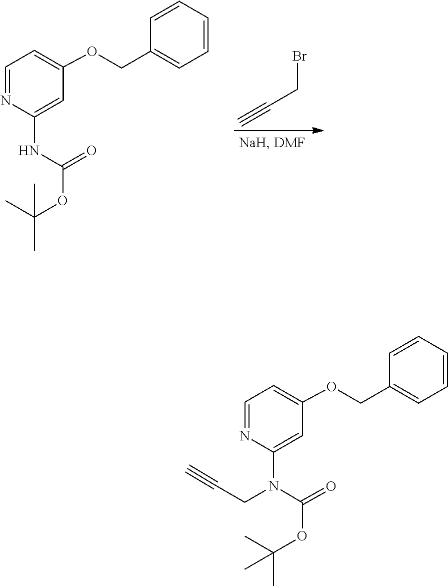

To a solution of tert-butyl N-[4-(benzyloxy)pyridin-2-yl]carbamate (2.5 g, 8.19 mmol) in DMF (20.0 mL) were added NaH (294.6 mg, 60%) at 0° C. under N₂. The mixture was stirred at 0° C. for 30 min under N₂. Then propargyl bromide (1460.6 mg, 12.28 mmol) was added to the mixture. The resulting mixture was stirred at room temperature for 2 h. After the reaction was completed, the mixture was diluted with H₂O and extracted with ethyl acetate. The combined organic layer was washed with brine, dried over anhydrous sodium sulfate and filtered. The filtrate was concentrated under reduced pressure to afford tert-butyl N-[4-(benzyloxy)pyridin-2-yl]-N-(prop-2-yn-1-yl)carbamate (3.4 g, crude) as a yellow solid. LCMS (ESI, m/z): [M+H]⁺=339.2.

### Step 3. Synthesis of 7-(benzyloxy)-3-methylimidazo[1,2-a]pyridine

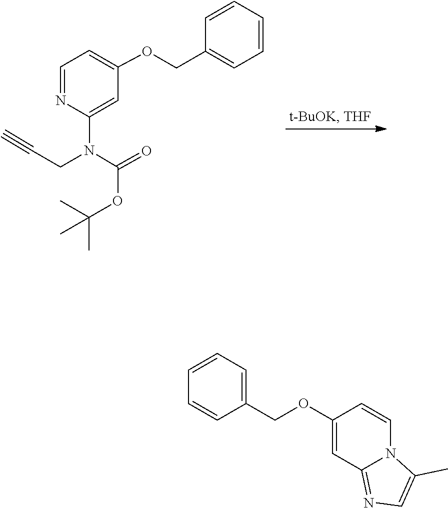 [SMILES: C#CCN(C(=O)OC(C)(C)C)c1cc(OCc2ccccc2)ccn1 ; Cc1cnc2cc(OCc3ccccc3)ccn12]

To a solution of tert-butyl N-[4-(benzyloxy)pyridin-2-yl]-N-(prop-2-yn-1-yl)carbamate (3.3 g, 9.75 mmol) in THE (40.0 mL) was added t-BuOK (1313.1 mg, 11.70 mmol) at room temperature. The resulting mixture was stirred at room temperature for 30 min. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with dichloromethane/methanol (90/10, v/v) to afford 7-(benzyloxy)-3-methylimidazo[1,2-a]pyridine (1.5 g, 66%) as a yellow solid. LCMS (ESI, m/z): [M+H]⁺=239.1.

### Step 4. Synthesis of 3-methylimidazo[1,2-a]pyridin-7-ol

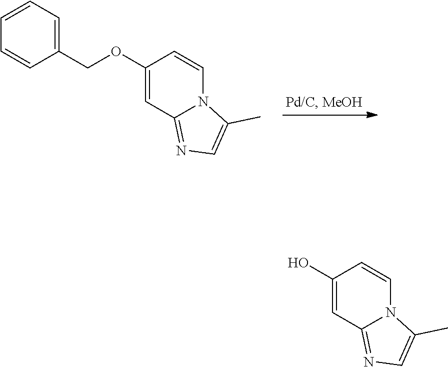 [SMILES: Cc1cnc2cc(OCc3ccccc3)ccn12 ; Cc1cnc2cc(O)ccn12]

To a solution of 7-(benzyloxy)-3-methylimidazo[1,2-a]pyridine (1.4 g, 6.04 mmol) in methanol (40.0 mL) was added Pd/C (578.8 mg, 10%) at room temperature under N₂. The resulting mixture was stirred at room temperature for 4 h under H₂. After the reaction was completed, the resulting mixture was filtered. The filtrate was concentrated under reduced pressure to afford 3-methylimidazo[1,2-a]pyridin-7-ol (895.0 mg, crude) as a brown solid. LCMS (ESI, m/z): [M+H]⁺=149.1.

### Step 5. Synthesis of 3-methyl-7-(2-methyl-4-nitrophenoxy)imidazo[1,2-a]pyridine

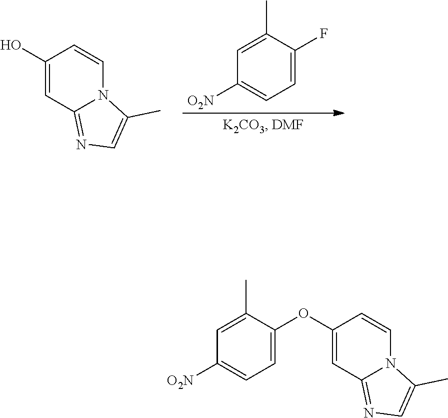 [SMILES: Cc1cnc2cc(O)ccn12 ; Cc1cc([N+](=O)[O-])ccc1F ; Cc1cc([N+](=O)[O-])ccc1Oc1ccn2c(C)cnc2c1]

To a solution of 3-methylimidazo[1,2-a]pyridin-7-ol (845.0 mg, 5.70 mmol) in DMF (5.0 mL) was added K₂CO₃ (1576.4 mg, 11.41 mmol) and 1-fluoro-2-methyl-4-nitrobenzene (884.7 mg, 5.70 mmol) at room temperature. The resulting mixture was stirred at 50° C. for 16 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with dichloromethane/methanol (95/5, v/v) to afford 3-methyl-7-(2-methyl-4-nitrophenoxy)imidazo[1,2-a]pyridine (644.0 mg, 39%) as a brown solid. LCMS (ESI, m/z): [M+H]⁺=284.1

### Step 6. Synthesis of 3-methyl-4-({3-methylimidazo[1,2-a]pyridin-7-yl}oxy)aniline

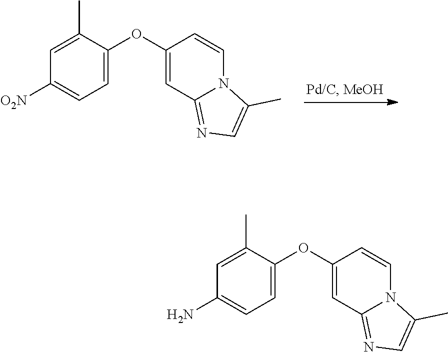 [SMILES: Cc1cc([N+](=O)[O-])ccc1Oc1ccn2c(C)cnc2c1 ; Cc1cc(N)ccc1Oc1ccn2c(C)cnc2c1]

To a solution of 3-methyl-7-(2-methyl-4-nitrophenoxy)imidazo[1,2-a]pyridine (593.6 mg, 2.09 mmol) in methanol (30.0 mL) was added Pd/C (260.0 mg, 10%) at room temperature under N₂. The resulting mixture was stirred at room temperature for 4 h under H₂. After the reaction was completed, the resulting mixture was filtered. The filtrate was concentrated under reduced pressure to afford 3-methyl-4-({3-methylimidazo[1,2-a]pyridin-7-yl}oxy)aniline (530.0 mg, crude) as a brown solid. LCMS (ESI, m/z): [M+H]⁺=254.1.

### Step 7. Synthesis of 6-chloro-N-[3-methyl-4-({3-methylimidazo[1,2-a]pyridin-7-yl}oxy)phenyl]pyrido[3,4-d]pyrimidin-4-amine

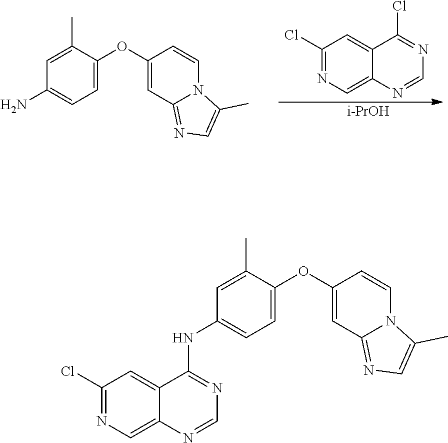 [SMILES: "i-PrOH" ; Cc1cc(N)ccc1Oc1ccn2c(C)cnc2c1 ; Clc1cc2c(Cl)ncnc2cn1 ; Cc1cc(Nc2ncnc3cnc(Cl)cc23)ccc1Oc1ccn2c(C)cnc2c1]

To a solution of 3-methyl-4-({3-methylimidazo[1,2-a]pyridin-7-yl}oxy)aniline (160.0 mg, 0.63 mmol) in isopropyl alcohol (5.0 mL) was added 4,6-dichloropyrido[3,4-d]pyrimidine (126.3 mg, 0.63 mmol) at room temperature. The resulting solution was stirred at 50° C. for 16 h. After the reaction was completed, the mixture was concentrated under reduced pressure. The residue was purified by flash column chromatography with dichloromethane/methanol (85/15, v/v) to afford 6-chloro-N-[3-methyl-4-({3-methylimidazo[1,2-a]pyridin-7-yl}oxy)phenyl]pyrido[3,4-d]pyrimidin-4-amine (195.1 mg, 75%) as a yellow solid. LCMS (ESI, m/z): [M+H]⁺=417.1.

### Step 8. Synthesis of (3E)-3-[2-(dimethylamino)ethylidene]-1-(4-{[3-methyl-4-({3-methylimidazo[1,2-a]pyridin-7-yl}oxy)phenyl]amino}pyrido[3,4-d]pyrimidin-6-yl)pyrrolidin-2-one (Compound 82)

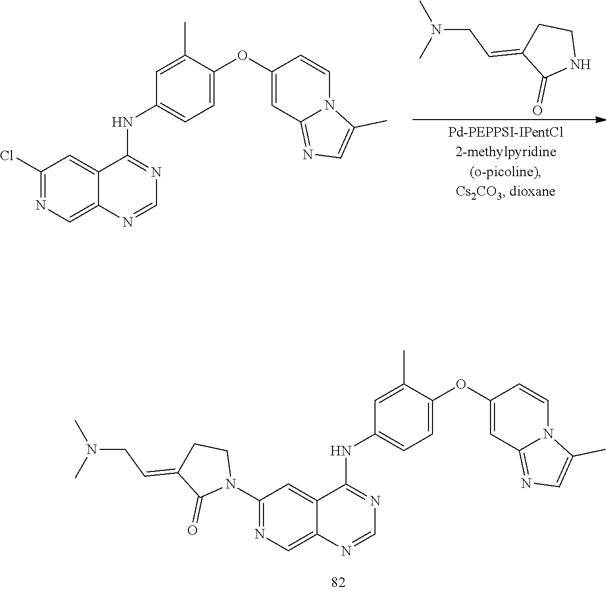

To a solution of 6-chloro-N-[3-methyl-4-({3-methylimidazo[1,2-a]pyridin-7-yl}oxy)phenyl]pyrido[3,4-d]pyrimidin-4-amine (306.0 mg, 0.73 mmol) in 1,4-dioxane (5.0 mL) was added Cs₂CO₃ (717.5 mg, 2.20 mmol), (3E)-3-[2-(dimethylamino)ethylidene]pyrrolidin-2-one (135.8 mg, 0.88 mmol) and Pd-PEPPSI-IPentCl 2-methylpyridine (o-picoline) (61.7 mg, 0.07 mmol) at room temperature under N₂. The resulting mixture was stirred at 100° C. for 2 h. After the reaction was completed, the resulting mixture was filtered. The filtrate was concentrated under vacuum. The residue was purified by flash chromatography with CH₂Cl₂/MeOH (85/15, v/v) and then purified by Prep-HPLC with the following conditions (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 30% B to 45% B in 8 min; Wave Length: 254 nm) to afford (3E)-3-[2-(dimethylamino)ethylidene]-1-(4-{[3-methyl-4-({3-methylimidazo[1,2-a]pyridin-7-yl}oxy)phenyl]amino}pyrido[3,4-d]pyrimidin-6-yl)pyrrolidin-2-one (Compound 82) (17.5 mg, 4%) as a white solid. LCMS (ESI, m/z): [M+H]⁺=535.3. ¹H NMR (400 MHz, DMSO-d₆): δ 10.32 (s, 1H), 9.22 (s, 1H), 9.09 (s, 1H), 8.64 (s, 1H), 8.28 (s, 1H), 7.82-7.76 (m, 2H), 7.23 (s, 1H), 7.16-7.13 (m, 1H), 6.87-6.81 (m, 1H), 6.59-6.50 (m, 2H), 4.20-4.16 (m, 2H), 3.15-3.10 (m, 2H), 2.91-2.85 (m, 2H), 2.48 (s, 3H), 2.22 (s, 9H).

### Example S77: Synthesis of (Z)-3-(2-(dimethylamino)ethylidene)-1-(4-((3-methyl-4-((1-methyl-1H-benzo[d]imidazol-5 yl)oxy)phenyl)amino)pyrido[3,2-d]pyrimidin-6-yl)pyrrolidin-2-one (Compound 83)

### Step 1. Synthesis of (Z)-3-(2-(methylamino)ethylidene)pyrrolidin-2-one trifluoracetate

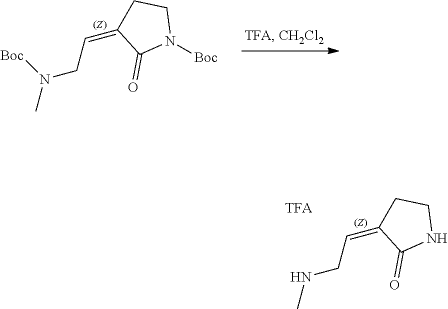

To a solution of tert-butyl (Z)-3-(2-((tert-butoxycarbonyl)(methyl)amino)ethylidene)-2-oxopyrrolidine-1-carboxylate (4.0 g, 11.75 mmol) in DCM (30.0 mL) was added TFA (10.0 mL) at 0° C. The resulting mixture was stirred at room temperature for 1 h. After the reaction was completed, the resulting mixture was concentrated under reduced pressure to afford (Z)-3-(2-(methylamino)ethylidene)pyrrolidin-2-one trifluoracetate (5.0 g, crude) as an black oil. LCMS (ESI, m/z): [M+H]⁺=141.0.

### Step 2. Synthesis of (Z)-3-(2-(dimethylamino)ethylidene)pyrrolidin-2-one

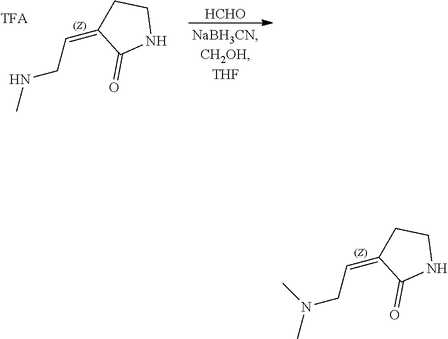

To a mixture of (Z)-3-(2-(methylamino)ethylidene)pyrrolidin-2-one trifluoracetate (5.0 g, crude) in THF (40.0 mL) and MeOH (10.0 mL) was added formaldehyde (3.4 g, 37% in H₂O) at room temperature. The resulting mixture was stirred at room temperature for 3 h. Then NaBH₃CN (11.2 g, 178.33 mmol) was added to the mixture at 0° C. The resulting mixture was stirred at room temperature for additional 3 h. After the reaction was completed, the resulting mixture was concentrated under reduced pressure. The residue was purified by reverse phase flash column chromatography with methanol/H₂O (60/40, v/v) to afford (Z)-3-(2-(dimethylamino)ethylidene)pyrrolidin-2-one (0.2 g, 3%) as a yellow oil. LCMS (ESI, m/z): [M+H]⁺=155.1.

### Step 3. Synthesis of (Z)-3-(2-(dimethylamino)ethylidene)-1-(4-((3-methyl-4-((1-methyl-1H-benzo[d]imidazol-5-yl)oxy)phenyl)amino)pyrido[3,2-d]pyrimidin-6-yl)pyrrolidin-2-one (Compound 83)

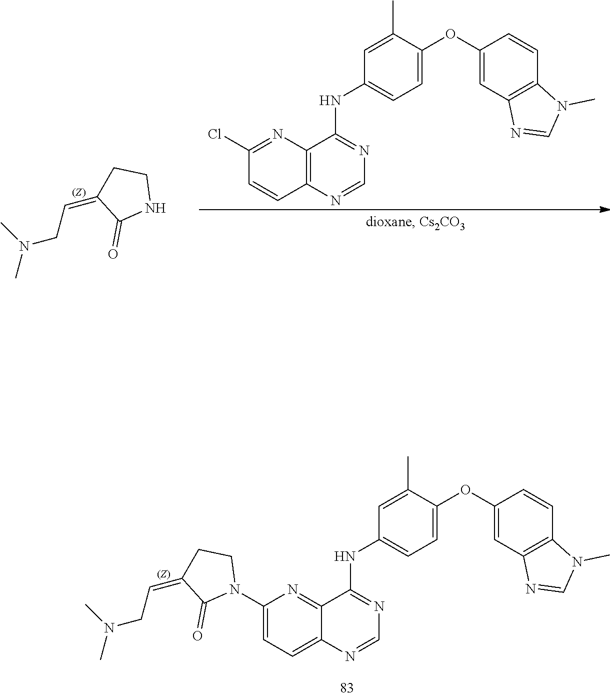

To a mixture of 6-chloro-N-(3-methyl-4-((1-methyl-1H-benzo[d]imidazol-5-yl)oxy)phenyl)pyrido[3,2-d]pyrimidin-4-amine (250.0 mg, 0.60 mmol) and (Z)-3-(2-(dimethylamino)ethylidene)pyrrolidin-2-one (184.9 mg, 1.20 mmol) in dioxane (10.0 mL) were added Cs₂CO₃ (586.1 mg, 1.80 mmol), Pd(OAc)₂ (13.4 mg, 0.06 mmol) and XPhos (57.1 mg, 0.10 mmol) at room temperature under N₂. The resulting mixture was stirred at 80° C. for 16 h. After the reaction was completed, the resulting mixture was diluted with water and extracted with ethyl acetate. The combined organic layers were washed with brine, dried over anhydrous Na₂SO₄ and filtered. The filtrate was concentrated under reduced pressure. The residue was purified by reverse phase flash column chromatography with methanol/H₂O (80/20, v/v) and then purified by Prep-HPLC with the following conditions: (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 32% B to 42% B in 8 min; Wave Length: 254 nm) to afford (Z)-3-(2-(dimethylamino)ethylidene)-1-(4-((3-methyl-4-((1-methyl-1H-benzo[d]imidazol-5-yl)oxy)phenyl)amino)pyrido[3,2-d]pyrimidin-6-yl)pyrrolidin-2-one (Compound 83) (10.2 mg, 3%) as a yellow solid. LCMS (ESI, m/z): [M+H]⁺=535.2. ¹H NMR (400 MHz, DMSO-d₆): δ 9.50 (s, 1H), 8.92 (d, J=9.2 Hz, 1H), 8.58 (s, 1H), 8.23-8.18 (m, 2H), 7.83 (s, 1H), 7.79-7.77 (m, 1H), 7.57 (d, J=8.8 Hz, 1H), 7.10 (d, J=2.0 Hz, 1H), 7.02-6.99 (m, 1H), 6.91 (d, J=8.8 Hz, 1H), 6.22-6.19 (m, 1H), 4.32-4.28 (m, 2H), 3.85 (s, 3H), 3.59 (d, J=6.0 Hz, 2H), 2.91-2.83 (m, 2H), 2.27 (s, 3H), 2.20 (s, 6H).

### Example S78: Synthesis of 1-[7-({3-methyl-4-[(1-methyl-1,3-benzodiazol-5-yl)oxy]phenyl}amino)[1,3]thiazolo[5,4-d]pyrimidin-2 yl]-3-methylidenepyrrolidin-2-one (Compound 84)

### Step 1. Synthesis of N-{7-chloro-[1,3]thiazolo[5,4-d]pyrimidin-2-yl}benzamide

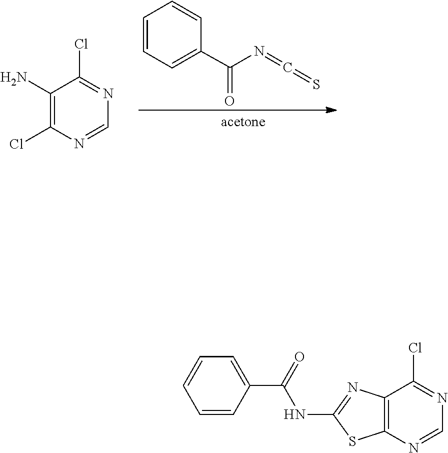 [SMILES: Nc1c(Cl)ncnc1Cl ; O=C(N=C=S)c1ccccc1 ; O=C(Nc1nc2c(Cl)ncnc2s1)c1ccccc1]

To a solution of 4,6-dichloropyrimidin-5-amine (20.0 g, 121.95 mmol) in acetone (200.0 mL) was added benzoyl isothiocyanate (21.8 g, 134.15 mmol) at room temperature. The resulting mixture was stirred at 60° C. for 6 h. After the reaction was completed, the mixture was cooled to room temperature and filtered. The solid was washed with acetone and collected to afford N-{7-chloro-[1,3]thiazolo[5,4-d]pyrimidin-2-yl}benzamide (29.0 g, crude) as a light yellow solid. LCMS (ESI, m/z): [M+H]⁺=291.0.

### Step 2. Synthesis of N-[7-({3-methyl-4-[(1-methyl-1,3-benzodiazol-5-yl)oxy]phenyl}amino)-[1,3]thiazolo[5,4-d]pyrimidin-2-yl]benzamide

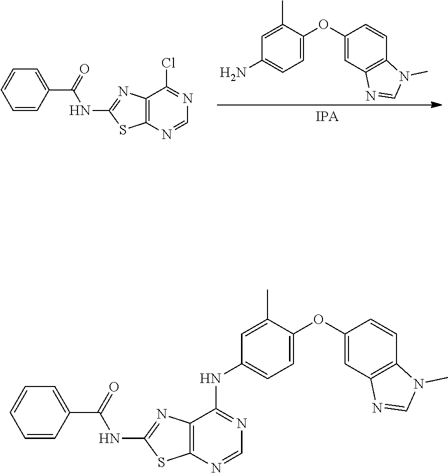 [SMILES: O=C(Nc1nc2c(Cl)ncnc2s1)c1ccccc1 ; Cc1cc(N)ccc1Oc1ccc2c(c1)ncn2C ; Cc1cc(Nc2ncnc3sc(NC(=O)c4ccccc4)nc23)ccc1Oc1ccc2c(c1)ncn2C]

To a mixture of N-{7-chloro-[1,3]thiazolo[5,4-d]pyrimidin-2-yl}benzamide (5.0 g, 17.19 mmol) in IPA (100.0 mL) was added 3-methyl-4-[(1-methyl-1,3-benzodiazol-5-yl)oxy]anilin (4.3 g, 17.19 mmol) at 0° C. The resulting mixture was stirred at 80° C. for 16 h.

After the reaction was completed, the mixture was concentrated under reduced pressure. The residue was purified by flash column chromatography with petroleum ether/ethyl acetate (1/1, v/v) to afford N-[7-({3-methyl-4-[(1-methyl-1,3-benzodiazol-5-yl)oxy]phenyl}amino)-[1,3]thiazolo[5,4-d]pyrimidin-2-yl]benzamide (5.0 g, 57%) as a yellow solid. LCMS (ESI, m/z): [M+H]⁺=508.1.

### Step 3. Synthesis of N7-{3-methyl-4-[(1-methyl-1,3-benzodiazol-5-yl)oxy]phenyl}-[1,3]thiazolo[5,4-d]pyrimidine-2,7-diamine

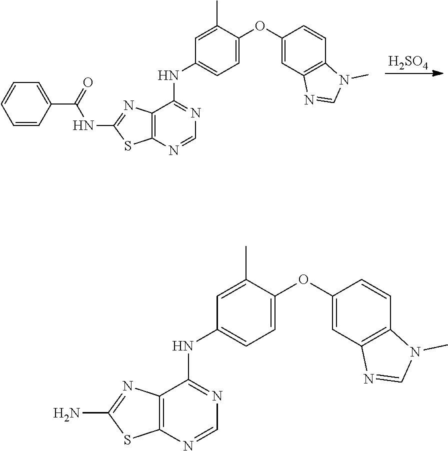 [SMILES: Cc1cc(Nc2ncnc3sc(NC(=O)c4ccccc4)nc23)ccc1Oc1ccc2c(c1)ncn2C ; "H2SO4" ; Cc1cc(Nc2ncnc3sc(N)nc23)ccc1Oc1ccc2c(c1)ncn2C]

A solution of N-[7-({3-methyl-4-[(1-methyl-1,3-benzodiazol-5-yl)oxy]phenyl}amino)-[1,3]thiazolo[5,4-d]pyrimidin-2-yl]benzamide (5.0 g, 9.85 mmol) in H₂SO₄ (50.0 mL, 70%) was stirred at 100° C. for 16 h. After the reaction was completed, the reaction mixture was quenched with sat. NaOH (aq.) at 0° C. The reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford N7-{3-methyl-4-[(1-methyl-1,3-benzodiazol-5-yl)oxy]phenyl}-[1,3]thiazolo[5,4-d]pyrimidine-2,7-diamine (1.4 g, 35%) as a yellow solid. LCMS (ESI, m/z): [M+H]⁺=404.1.

### Step 4. Synthesis of 2-bromo-N-{3-methyl-4-[(1-methyl-1,3-benzodiazol-5-yl)oxy]phenyl}-[1,3]thiazolo[5,4-d]pyrimidin-7-amine

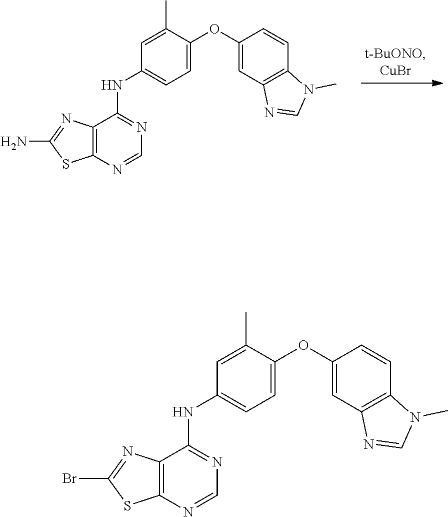 [SMILES: Cc1cc(Nc2ncnc3sc(N)nc23)ccc1Oc1ccc2c(c1)ncn2C ; Cc1cc(Nc2ncnc3sc(Br)nc23)ccc1Oc1ccc2c(c1)ncn2C]

To a solution of N7-{3-methyl-4-[(1-methyl-1,3-benzodiazol-5-yl)oxy]phenyl}-[1,3]thiazolo[5,4-d]pyrimidine-2,7-diamine (750.0 mg, 1.85 mmol) in ACN (5.0 mL)/H₂O (5.0 mL) was added CuBr (399.9 mg, 2.78 mmol) and t-BuONO (0.2 mL, 1.85 mmol) at room temperature. The mixture was irradiated with microwave radiation at room temperature for 0.5 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford 2-bromo-N-{3-methyl-4-[(1-methyl-1,3-benzodiazol-5-yl)oxy]phenyl}-[1,3]thiazolo[5,4-d]pyrimidin-7-amine (50.0 mg, 5%) as a yellow solid. LCMS (ESI, m/z): [M+H]⁺=467.0.

### Step 5. Synthesis of 1-[7-({3-methyl-4-[(1-methyl-1,3-benzodiazol-5-yl)oxy]phenyl}amino)-[1,3]thiazolo[5,4-d]pyrimidin-2-yl]-3-methylidenepyrrolidin-2-one (Compound 84)

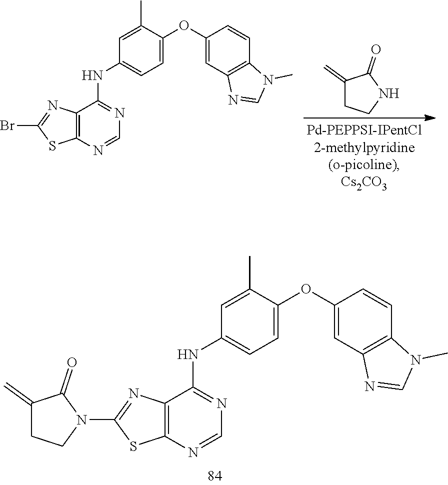

To a solution of 2-bromo-N-{3-methyl-4-[(1-methyl-1,3-benzodiazol-5-yl)oxy]phenyl}-[1,3]thiazolo[5,4-d]pyrimidin-7-amine (50.0 mg, 0.10 mmol) in dioxane (3.0 mL) was added 3-methylidenepyrrolidin-2-one (41.5 mg, 0.4 mmol), Pd-PEPPSI-IPentCl 2-methylpyridine (o-picoline) (18.0 mg, 0.02 mmol) and Cs₂CO₃ (104.5 mg, 0.32 mmol) at room temperature under N₂. The final reaction mixture was irradiated with microwave radiation at 100° C. for 1 h. After the reaction was completed, the mixture was filtered. The filtrate was evaporated in vacuo. The residue was purified by flash column chromatography with CH₂Cl₂/MeOH (10/1, v/v) and then purified by Prep-HPLC with the following conditions: (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 22% B to 27% B in 8 min; Wave Length: 254 nm) to afford 1-[7-({3-methyl-4-[(1-methyl-1,3-benzodiazol-5-yl)oxy]phenyl}amino)-[1,3]thiazolo[5,4-d]pyrimidin-2-yl]-3-methylidenepyrrolidin-2-one (Compound 84) (2.9 mg, 5%) as an off-white solid. LCMS (ESI, m/z): [M+H]⁺=484.3. ¹H NMR (400 MHz, DMSO-d₆): δ 9.50 (s, 1H), 8.47 (s, 1H), 8.17 (s, 1H), 7.78 (d, J=2.0 Hz, 1H), 7.68-7.65 (m, 1H), 7.57 (d, J=8.8 Hz, 1H), 7.08 (d, J=2.4 Hz, 1H), 7.01-6.98 (m, 1H), 6.86 (d, J=8.8 Hz, 1H), 6.14 (s, 1H), 5.73 (s, 1H), 4.27-4.24 (m, 2H), 3.84 (s, 3H), 3.05-3.01 (m, 2H), 2.24 (s, 3H).

### Example S79: Synthesis of (1S,5S)-2-(4-((4-([1,2,4]triazolo[1,5-a]pyridin-7 ylmethyl)-3-methylphenyl)amino)quinazolin-6-yl)-4-methylene-2-azabicyclo[3.1.0]hexan-3-one and (1R,5R)-2-(4-((4-([1,2,4]triazolo[1,5-a]pyridin-7-ylmethyl)-3-methylphenyl)amino)quinazolin-6-yl)-4-methylene-2-azabicyclo[3.1.0]hexan-3-one (Compound 85 and Compound 86)

### Step 1. Synthesis of N-(4-([1,2,4]triazolo[1,5-a]pyridin-7-ylmethyl)-3-methylphenyl)-6-iodoquinazolin-4-amine

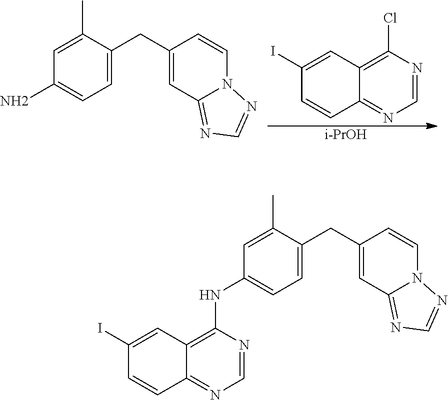

To a solution of 4-([1,2,4]triazolo[1,5-a]pyridin-7-ylmethyl)-3-methylaniline (260.0 mg, 1.10 mmol) in isopropanol (10.0 mL) was added 4-chloro-6-iodoquinazoline (316.9 mg, 1.10 mmol) at room temperature. The resulting mixture was stirred at 30° C. for 16 h. After the reaction was completed, the resulting mixture was concentrated under reduced pressure. The residue was purified by flash column with dichloromethane/methanol (85/15, v/v) to afford N-(4-([1,2,4]triazolo[1,5-a]pyridin-7-ylmethyl)-3-methylphenyl)-6-iodoquinazolin-4-amine (130.0 mg, 24%) as a white solid. LCMS (ESI, m/z): [M+H]⁺=493.1.

### Step 2. Synthesis of 2-(4-((4-([1,2,4]triazolo[1,5-a]pyridin-7-ylmethyl)-3-methylphenyl)amino)quinazolin-6-yl)-4-methylene-2-azabicyclo[3.1.0]hexan-3-one

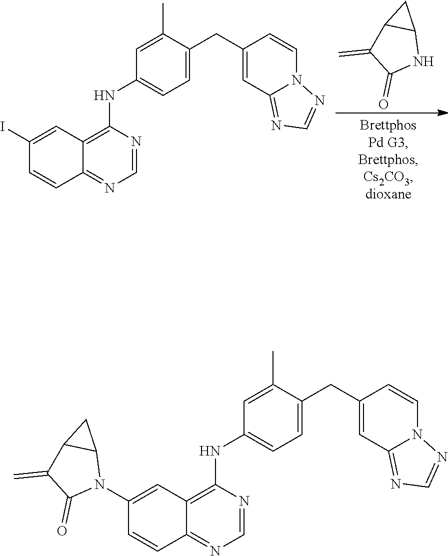

To a solution of N-(4-([1,2,4]triazolo[1,5-a]pyridin-7-ylmethyl)-3-methylphenyl)-6-iodoquinazolin-4-amine (70.0 mg, 0.14 mmol) in dioxane (5.0 mL) was added 4-methylene-2-azabicyclo[3.1.0]hexan-3-one (46.5 mg, 0.43 mmol), Cs₂CO₃ (171.1 mg, 0.45 mmol), BrettPhos (30.5 mg, 0.06 mmol) and Brettphos Pd G3 (25.8 mg, 0.03 mmol) at room temperature under N₂. The resulting mixture was stirred at 100° C. for 2 h under N₂. After the reaction was completed, the resulting mixture was filtered. The filtrate was concentrated under reduced pressure. The residue was purified by flash chromatography with CH₂Cl₂/MeOH (90/10, v/v) to afford 2-(4 ((4-([1,2,4]triazolo[1,5-a]pyridin-7-ylmethyl)-3-methylphenyl)amino)quinazolin-6-yl)-4-methylene-2-azabicyclo[3.1.0]hexan-3-one (40.0 mg, 60%) as a light yellow solid. LCMS (ESI, m/z): [M+H]⁺=474.2.

### Step 3. Separation of (1S,5S)-2-(4-((4-([1,2,4]triazolo[1,5-a]pyridin-7-ylmethyl)-3-methylphenyl)amino)quinazolin-6-yl)-4-methyl ene-2-azabicyclo[3.1.0]hexan-3-one and (1R,5R)-2-(4-((4-([1,2,4]triazolo[1,5-a]pyridin-7-ylmethyl)-3-methylphenyl)amino)quinazolin-6-yl)-4-methyl ene-2-azabicyclo[3.1.0]hexan-3-one

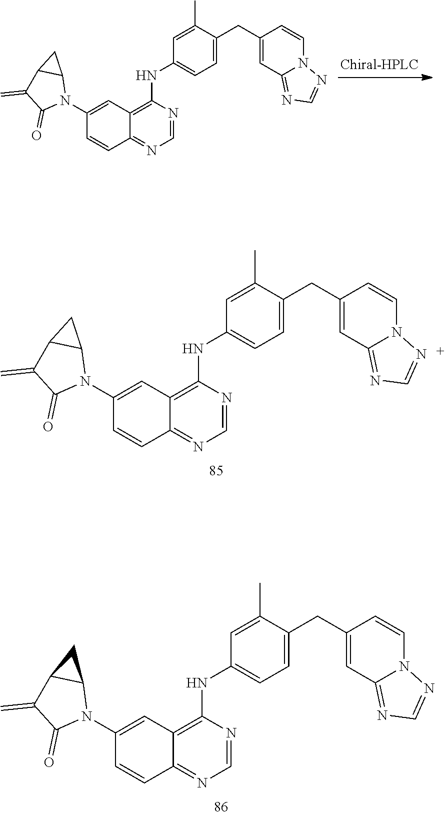 [SMILES: C=C1C(=O)N(c2ccc3ncnc(Nc4ccc(Cc5ccn6ncnc6c5)c(C)c4)c3c2)C2CC12 ; C=C1C(=O)N(c2ccc3ncnc(Nc4ccc(Cc5ccn6ncnc6c5)c(C)c4)c3c2)C2CC12 ; C=C1C(=O)N(c2ccc3ncnc(Nc4ccc(Cc5ccn6ncnc6c5)c(C)c4)c3c2)[C@@H]2C[C@H]12]

The racemic mixture of 2-(4-((4-([1,2,4]triazolo[1,5-a]pyridin-7-ylmethyl)-3-methylphenyl)amino)quinazolin-6-yl)-4-methylene-2-azabicyclo[3.1.0]hexan-3-one (40.0 mg, 0.08 mmol) was separated by Prep-Chiral-HPLC with the following conditions (Column: CHIRALPAK IG, 2×25 cm, 5 μm; Mobile Phase A: Hex (0.5% 2M NH₃-MeOH)-HPLC, Mobile Phase B: MeOH: DCM=1: 1; Flow rate: 20 mL/min; Gradient: 55% B to 55% B in 20 min; Wave Length: 220/254 nm; RT1(min): 11.71; RT2(min): 16.71) to afford 2-(4-((4-([1,2,4]triazolo[1,5-a]pyridin-7-ylmethyl)-3-methylphenyl)amino)quinazolin-6-yl)-4-methylene-2-azabicyclo[3.1.0]hexan-3-one Enantiomer 1 (retention time 11.71 minutes, 3.2 mg, 16%) as a white solid and 2-(4-((4-([1,2,4]triazolo[1,5-a]pyridin-7-ylmethyl)-3-methylphenyl)amino)quinazolin-6-yl)-4-methylene-2-azabicyclo[3.1.0]hexan-3-one Enantiomer 2 (retention time 16.71 minutes, 6.1 mg, 30%) as a white solid. The absolute stereochemistry of Enantiomers 1 and 2 was not assigned. The two enantiomeric structures that could be obtained from chiral separation of the enantiomeric mixture as described above are shown as Compounds 85 and 86 in Table 1.

2-(4-((4-([1,2,4]triazolo[1,5-a]pyridin-7-ylmethyl)-3-methylphenyl)amino)quinazolin-6-yl)-4-methylene-2-azabicyclo[3.1.0]hexan-3-one Enantiomer 1: RT1(min): 11.71; LCMS (ESI, m/z): [M+H]⁺=474.3. ¹H NMR (400 MHz, DMSO-d₆): δ 9.83 (s, 1H), 8.87 (d, J=6.8 Hz, 1H), 8.57-8.54 (m, 2H), 8.44 (s, 2H), 7.84 (d, J=8.8 Hz, 1H), 7.67-7.53 (m, 3H), 7.28 (d, J=8.0 Hz, 1H), 7.03 (d, J=6.4 Hz, 1H), 6.00 (s, 1H), 5.74 (s, 1H), 4.15-4.06 (m, 3H), 2.55-2.51 (m, 1H), 2.28 (s, 3H), 1.46-1.42 (m, 1H), 0.99-0.91 (m, 1H).

2-(4-((4-([1,2,4]triazolo[1,5-a]pyridin-7-ylmethyl)-3-methylphenyl)amino)quinazolin-6-yl)-4-methylene-2-azabicyclo[3.1.0]hexan-3-one Enantiomer 2: RT2(min): 16.71; LCMS (ESI, m/z): [M+H]⁺=474.2 ¹H NMR (400 MHz, DMSO-d₆): δ 9.83 (s, 1H), 8.87 (d, J=7.2 Hz, 1H), 8.57-8.54 (m, 2H), 8.46-8.44 (m, 2H), 7.84 (d, J=8.8 Hz, 1H), 7.67-7.61 (m, 2H), 7.53 (s, 1H), 7.28 (d, J=8.0 Hz, 1H), 7.04-7.02 (m, 1H), 6.00 (s, 1H), 5.74 (s, 1H), 4.15 (s, 2H), 4.07-4.05 (m, 1H), 2.55-2.51 (m, 1H), 2.28 (s, 3H), 1.46-1.44 (m, 1H), 0.99-0.93 (m, 1H).

### Example S80: Synthesis of 1-(4-((3-methyl-4-((1-methyl-1H-benzo[d]imidazol-5-yl)oxy)phenyl)amino)pyrido[3,2-d]pyrimidin-6-yl)-3-methylenepyrrolidin-2-one; formic acid (Compound 87)

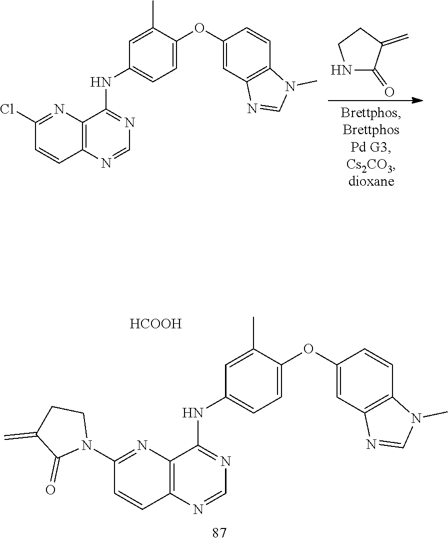

To a solution of 6-chloro-N-{3-methyl-4-[(1-methyl-1,3-benzodiazol-5-yl)oxy]phenyl}pyrido[3,2-d]pyrimidin-4-amine (0.3 g, 0.72 mmol) in 1,4-dioxane (10.0 mL) was added Cs₂CO₃ (0.3 g, 2.16 mmol), Brettphos (77.3 mg, 0.14 mmol), Brettphos Pd G3 (65.3 mg, 0.07 mmol) and 3-methylidenepyrrolidin-2-one (69.8 mg, 0.72 mmol) at room temperature under N₂. The resulting mixture was stirred at 80° C. for 16 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with dichloromethane/methanol (90/10, v/v) and then purified by Prep-HPLC with the following conditions: (Column: Xselect CSH C18 OBD Column 30×150 mm 5 μm; Mobile Phase A: ACN, Mobile Phase B: Water (0.1% FA); Flow rate: 60 mL/min; Gradient: 5% B to 25% B in 12 min; Wave Length: 254 nm) to afford 1-(4-((3-methyl-4-((1-methyl-1H-benzo[d]imidazol-5-yl)oxy)phenyl)amino)pyrido[3,2-d]pyrimidin-6-yl)-3-methylenepyrrolidin-2-one; formic acid (Compound 87) (3.5 mg, 1%). LCMS (ESI, m/z): [M+H]⁺=478.3. ¹H NMR (400 MHz, DMSO-d₆): δ 9.56 (s, 1H), 8.97 (d, J=9.2 Hz, 1H), 8.60 (s, 1H), 8.27 (d, J=9.2 Hz, 1H), 8.18 (d, J=8.8 Hz, 2H), 7.84-7.77 (m, 2H), 7.58 (d, J=8.8 Hz, 1H), 7.11 (d, J=2.4 Hz, 1H), 7.02-7.69 (m, 1H), 6.92 (d, J=8.8 Hz, 1H), 6.08 (d, J=2.8 Hz, 1H), 5.63 (s, 1H), 4.36-4.33 (m, 2H), 3.85 (s, 3H), 2.98-2.93 (m, 2H), 2.28 (s, 3H).

### Example S81: Synthesis of (Z)-3-(2-(dimethylamino)ethylidene)-1-(4-((3-methyl-4-((1-methyl-1H-benzo[d]imidazol-5 yl)oxy)phenyl)amino)pyrido[3,2-d]pyrimidin-6-yl)pyrrolidin-2-one (Compound 88)

### Step 1. Synthesis of (Z)-3-(2-(methylamino)ethylidene)pyrrolidin-2-one trifluoracetate

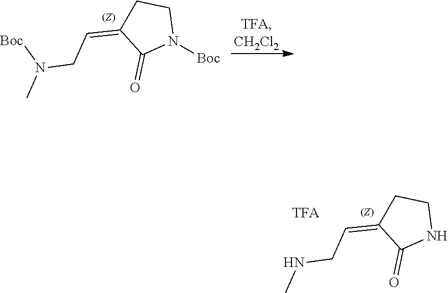

To a solution of tert-butyl (Z)-3-(2-((tert-butoxycarbonyl)(methyl)amino)ethylidene)-2-oxopyrrolidine-1-carboxylate (4.0 g, 11.75 mmol) in DCM (30.0 mL) was added TFA (10.0 mL) at 0° C. The resulting mixture was stirred at room temperature for 1 h. After the reaction was completed, the resulting mixture was concentrated under reduced pressure to afford (Z)-3-(2-(methylamino)ethylidene)pyrrolidin-2-one trifluoracetate (5.0 g, crude) as an black oil. LCMS (ESI, m/z): [M+H]⁺=141.0.

### Step 2. Synthesis of (Z)-3-(2-(dimethylamino)ethylidene)pyrrolidin-2-one

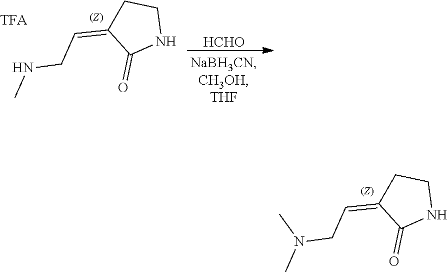

To a mixture of (Z)-3-(2-(methylamino)ethylidene)pyrrolidin-2-one trifluoracetate (5.0 g, crude) in THF (40.0 mL) and MeOH (10.0 mL) was added formaldehyde (3.4 g, 37% in H₂O) at room temperature. The resulting mixture was stirred at room temperature for 3 h. Then NaBH₃CN (11.2 g, 178.33 mmol) was added to the mixture at 0° C. The resulting mixture was stirred at room temperature for additional 3 h. After the reaction was completed, the resulting mixture was concentrated under reduced pressure. The residue was purified by reverse phase flash column chromatography with methanol/H₂O (60/40, v/v) to afford (Z)-3-(2-(dimethylamino)ethylidene)pyrrolidin-2-one (0.2 g, 3%) as a yellow oil. LCMS (ESI, m/z): [M+H]⁺=155.1.

### Step 3. Synthesis of (Z)-3-(2-(dimethylamino)ethylidene)-1-(4-((3-methyl-4-((1-methyl-1H-benzo[d]imidazol-5-yl)oxy)phenyl)amino)pyrido[3,2-d]pyrimidin-6-yl)pyrrolidin-2-one (Compound 88)

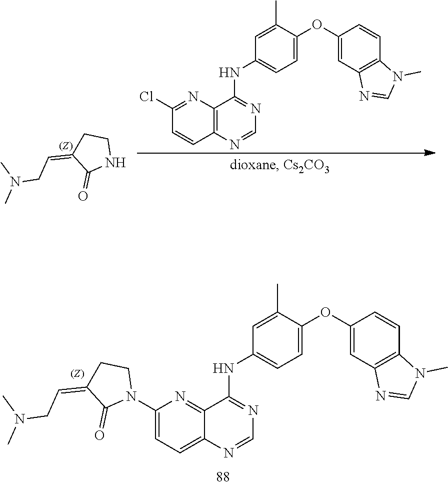

To a mixture of 6-chloro-N-(3-methyl-4-((1-methyl-1H-benzo[d]imidazol-5-yl)oxy)phenyl)pyrido[3,2-d]pyrimidin-4-amine (250.0 mg, 0.60 mmol) and (Z)-3-(2-(dimethylamino)ethylidene)pyrrolidin-2-one (184.9 mg, 1.20 mmol) in dioxane (10.0 mL) were added Cs₂CO₃ (586.1 mg, 1.80 mmol), Pd(OAc)₂ (13.4 mg, 0.06 mmol) and XPhos (57.1 mg, 0.10 mmol) at room temperature under N₂. The resulting mixture was stirred at 80° C. for 16 h. After the reaction was completed, the resulting mixture was diluted with water and extracted with ethyl acetate. The combined organic layers were washed with brine, dried over anhydrous Na₂SO₄ and filtered. The filtrate was concentrated under reduced pressure. The residue was purified by reverse phase flash column chromatography with methanol/H₂O (80/20, v/v) and then purified by Prep-HPLC with the following conditions: (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 32% B to 42% B in 8 min; Wave Length: 254 nm) to afford (Z)-3-(2-(dimethylamino)ethylidene)-1-(4-((3-methyl-4-((1-methyl-1H-benzo[d]imidazol-5-yl)oxy)phenyl)amino)pyrido[3,2-d]pyrimidin-6-yl)pyrrolidin-2-one (Compound 88) (10.2 mg, 3%) as a yellow solid. LCMS (ESI, m/z): [M+H]⁺=535.2. ¹H NMR (400 MHz, DMSO-d₆): δ 9.50 (s, 1H), 8.92 (d, J=9.2 Hz, 1H), 8.58 (s, 1H), 8.23-8.18 (m, 2H), 7.83 (s, 1H), 7.79-7.77 (m, 1H), 7.57 (d, J=8.8 Hz, 1H), 7.10 (d, J=2.0 Hz, 1H), 7.02-6.99 (m, 1H), 6.91 (d, J=8.8 Hz, 1H), 6.22-6.19 (m, 1H), 4.32-4.28 (m, 2H), 3.85 (s, 3H), 3.59 (d, J=6.0 Hz, 2H), 2.91-2.83 (m, 2H), 2.27 (s, 3H), 2.20 (s, 6H).

### Example S82: (S,E)-1-(4-((4-([1,2,4]triazolo[1,5-a]pyridin-7-yloxy)-3-methylphenyl)amino)pyrido[3,2-d]pyrimidin-6-yl)-3-((1-methylpyrrolidin-2-yl)methylene)pyrrolidin-2-one (Compound 89)

### Step 1. Synthesis of tert-butyl 3-(((S)-1-(tert-butoxycarbonyl)pyrrolidin-2-yl)(hydroxy)methyl)-2-oxopyrrolidine-1-carboxylate

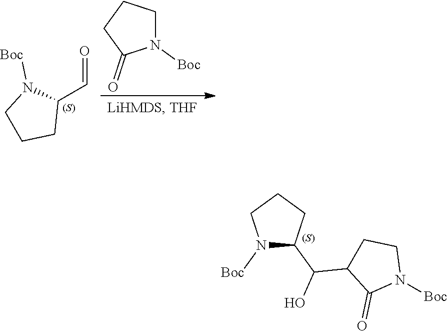

To a solution of tert-butyl 2-oxopyrrolidine-1-carboxylate (2.9 g, 16.06 mmol) in THE (80.0 mL) was added LiHMDS (35.0 mL, 1.0 mol/L) at −78° C. under N₂. The mixture was stirred at −78° C. for 1 h. Then a solution of tert-butyl (S)-2-formylpyrrolidine-1-carboxylate (5.0 g, 25.01 mmol) in THE (20.0 mL) was added to the mixture at −78° C. under N₂. The resulting mixture was stirred at −78° C. for 2 h. After the reaction was completed, the mixture was quenched with sat. NH₄Cl (aq.) and extracted with ethyl acetate. The combined organic layer was washed with brine, dried over anhydrous sodium sulfate and filtered. The filtrate was concentrated under reduced pressure to afford tert-butyl 3-(((S)-1-(tert-butoxycarbonyl)pyrrolidin-2-yl)(hydroxy)methyl)-2-oxopyrrolidine-1-carboxylate (4.3 g, crude) as a yellow oil. LCMS (ESI, m/z): [M+H]⁺=385.1.

### Step 2. Synthesis of tert-butyl 3-(((S)-1-(tert-butoxycarbonyl)pyrrolidin-2-yl)((methylsulfonyl)oxy)methyl)-2-oxopyrrolidine-1-carboxylate

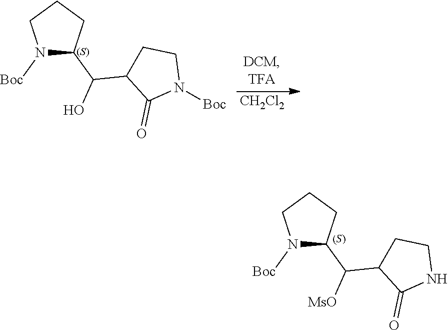

To a solution of tert-butyl 3-(((S)-1-(tert-butoxycarbonyl)pyrrolidin-2-yl)(hydroxy)methyl)-2-oxopyrrolidine-1-carboxylate (3.0 g, crude) in DCM (50.0 mL) was added TEA (3.1 g, 31.21 mmol) and MsCl (1.7 g, 15.60 mmol) at 0° C. The mixture was stirred at room temperature for 16 h. After the reaction was completed, the mixture was diluted with H₂O and extracted with ethyl acetate. The combined organic layer was washed with brine, dried over anhydrous sodium sulfate and filtered. The filtrate was concentrated under reduced pressure to afford tert-butyl 3-(((S)-1-(tert-butoxycarbonyl)pyrrolidin-2-yl)((methylsulfonyl)oxy)methyl)-2-oxopyrrolidine-1-carboxylate (2.5 g, crude) as a brown oil. LCMS (ESI, m/z): [M+H]⁺=463.1.

### Step 3. Synthesis of tert-butyl (S,E)-3-((1-(tert-butoxycarbonyl)pyrrolidin-2-yl)methylene)-2-oxopyrrolidine-1-carboxylate

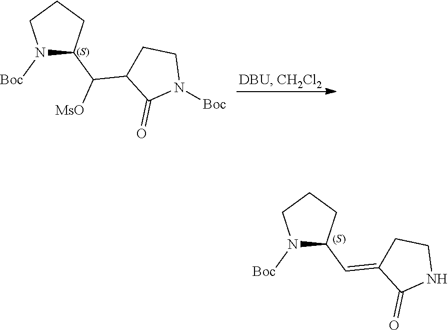

To a solution of tert-butyl 3-(((S)-1-(tert-butoxycarbonyl)pyrrolidin-2-yl)((methylsulfonyl)oxy)methyl)-2-oxopyrrolidine-1-carboxylate (2.0 g, crude) in DCM (20.0 mL) was added DBU (2.6 g, 17.29 mmol) at room temperature. The resulting mixture was stirred at room temperature for 2 h. After the reaction was completed, the mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (20/80, v/v) to afford tert-butyl (S,E)-3-((1-(tert-butoxycarbonyl)pyrrolidin-2-yl)methylene)-2-oxopyrrolidine-1-carboxylate (1.5 g, 94%) as a pink solid. LCMS (ESI, m/z): [M+H]⁺=367.1.

### Step 4. Synthesis of (S,E)-3-(pyrrolidin-2-ylmethylene)pyrrolidin-2-one; 2,2,2-trifluoroacetic acid

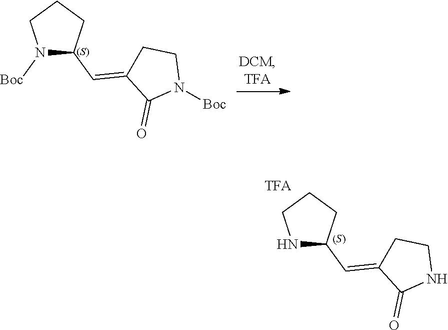

To a solution of tert-butyl (S,E)-3-((1-(tert-butoxycarbonyl)pyrrolidin-2-yl)methylene)-2-oxopyrrolidine-1-carboxylate (500.0 mg, 1.36 mmol) in DCM (5.0 mL) was added TFA (5.0 mL) at room temperature. The resulting mixture was stirred at room temperature for 2 h. After the reaction was completed, the resulting mixture was concentrated under reduced pressure to afford (S,E)-3-(pyrrolidin-2-ylmethylene)pyrrolidin-2-one; 2,2,2-trifluoroacetic acid (200.0 mg, crude) as a yellow solid. LCMS (ESI, m/z): [M+H]⁺=167.1

### Step 5. Synthesis of (S,E)-3-((1-methylpyrrolidin-2-yl)methylene)pyrrolidin-2-one

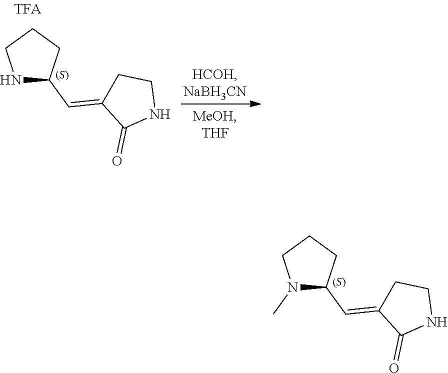

To a solution of (S,E)-3-(pyrrolidin-2-ylmethylene)pyrrolidin-2-one; 2,2,2-trifluoroacetic acid (1.4 g, crude) in THE (20.0 mL) and MeOH (4.0 mL) and HCHO (0.7 g, 40%) at room temperature. The resulting mixture was stirred at room temperature for 1 h. Then NaBH₃CN (2.3 g, 37.89 mmol) was added to the mixture. The resulting mixture was stirred at room temperature for 2 h. After the reaction was completed, the resulting mixture was quenched with water and concentrated under reduced pressure. The residue was purified by reverse phase flash chromatography with acetonitrile/water (30/70, v/v) to afford (S,E)-3-((1-methylpyrrolidin-2-yl)methylene)pyrrolidin-2-one (160.0 mg, 10%) as a yellow solid. LCMS (ESI, m/z): [M+H]⁺=181.1.

### Step 6. Synthesis of (S,E)-1-(4-((4-([1,2,4]triazolo[1,5-a]pyridin-7-yloxy)-3-methylphenyl)amino)pyrido[3,2-d]pyrimidin-6-yl)-3-((1-methylpyrrolidin-2-yl)methylene)pyrrolidin-2-one (Compound 89)

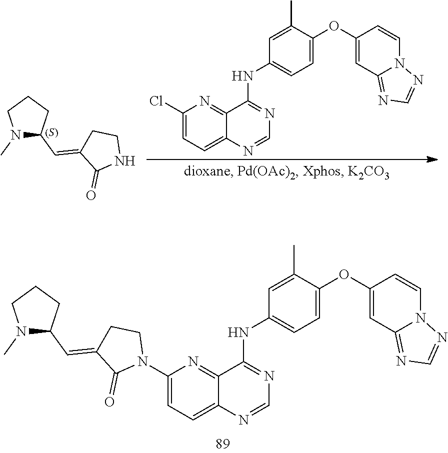

To a solution of (S,E)-3-((1-methylpyrrolidin-2-yl)methylene)pyrrolidin-2-one (142.8 mg, 0.79 mmol) in 1,4-dioxane (5.0 mL) was added K₂CO₃ (258.1 mg, 0.79 mmol), Pd(OAc)₂ (8.9 mg, 0.04 mmol), Xphos (37.8 mg, 0.79 mmol) and N-(4-([1,2,4]triazolo[1,5-a]pyridin-7-yloxy)-3-methylphenyl)-6-chloropyrido[3,2-d]pyrimidin-4-amine (160.0 mg, 0.39 mmol) at room temperature under N₂. The resulting mixture was stirred at 100° C. for 2 h. After the reaction was completed, the mixture was cooled to room temperature and filtered. The filtrate was concentrated under reduced pressure. The residue was purified by flash column chromatography with CH₂Cl₂/MeOH (90/10, v/v) to afford (S,E)-1-(4-((4-([1,2,4]triazolo[1,5-a]pyridin-7-yloxy)-3-methylphenyl)amino)pyrido[3,2-d]pyrimidin-6-yl)-3-((1-methylpyrrolidin-2-yl)methylene)pyrrolidin-2-one (Compound 89) (45.3 mg, 20%) as a yellow solid. LCMS (ESI, m/z): [M+H]⁺=548.4. ¹H NMR (400 MHz, DMSO-d₆): δ 9.59 (s, 1H), 9.00-8.94 (m, 2H), 8.64 (s, 1H), 8.39 (s, 1H), 8.27 (d, J=9.6 Hz, 1H), 8.00-7.97 (m, 2H), 7.26 (d, J=8.4 Hz, 1H), 7.05-7.03 (m, 1H), 6.81 (d, J=2.4 Hz, 1H), 6.43-6.40 (m, 1H), 4.37-4.34 (m, 2H), 3.07-3.03 (m, 1H), 2.94-2.88 (m, 3H), 2.22-2.16 (m, 7H), 2.07-2.01 (m, 1H), 1.81-1.77 (m, 2H), 1.60-1.56 (m, 1H).

### Example S83: Synthesis of (R,E)-1-(4-((4-([1,2,4]triazolo[1,5-a]pyridin-7-yloxy)-3-methylphenyl)amino)pyrido[3,2-d]pyrimidin-6-yl)-3-((1-methylpyrrolidin-2-yl)methylene)pyrrolidin-2-one (Compound 90)

### Step 1. Synthesis of tert-butyl 3-(((R)-1-(tert-butoxycarbonyl)pyrrolidin-2-yl)(hydroxy)methyl)-2-oxopyrrolidine-1-carboxylate

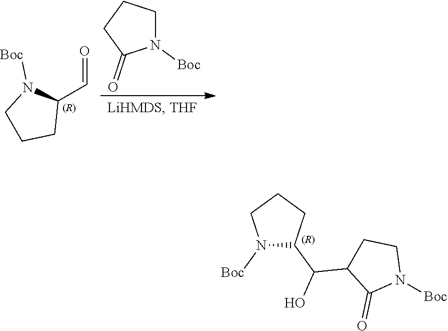

To a solution of tert-butyl 2-oxopyrrolidine-1-carboxylate (4.8 g, 25.70 mmol) in THF (80.0 mL) was added LiHMDS (50.0 mL, 1.0 mol/L) at −78° C. under N₂. The mixture was stirred at −78° C. for 1 h. Then a solution of tert-butyl (2R)-2-formylpyrrolidine-1-carboxylate (8.0 g, 40.151 mmol) in THF (30.0 mL) was added to the mixture at −78° C. under N₂. The resulting mixture was stirred at −78° C. for 2 h. After the reaction was completed, the mixture was quenched with sat. NH₄Cl (aq.) and extracted with ethyl acetate. The combined organic layer was washed with brine, dried over anhydrous sodium sulfate and filtered. The filtrate was concentrated under reduced pressure to afford tert-butyl 3-(((R)-1-(tert-butoxycarbonyl)pyrrolidin-2-yl)(hydroxy)methyl)-2-oxopyrrolidine-1-carboxylate (5.0 g, crude) as a yellow oil. LCMS (ESI, m/z): [M+H]⁺=385.2.

### Step 2. Synthesis of tert-butyl 3-(((R)-1-(tert-butoxycarbonyl)pyrrolidin-2-yl)((methylsulfonyl)oxy)methyl)-2-oxopyrrolidine-1-carboxylate

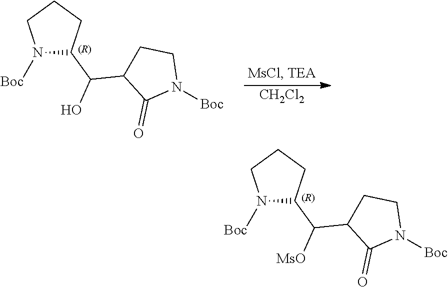

To a solution of tert-butyl 3-(((R)-1-(tert-butoxycarbonyl)pyrrolidin-2-yl)(hydroxy)methyl)-2-oxopyrrolidine-1-carboxylate (13.0 g, crude) in DCM (100.0 mL) was added TEA (10.3 g, 101.44 mmol) and MsCl (7.8 g, 67.63 mmol) at 0° C. The mixture was stirred at room temperature for 16 h. After the reaction was completed, the mixture was diluted with H₂O and extracted with CH₂Cl₂. The combined organic layer was washed with brine, dried over anhydrous sodium sulfate and filtered. The filtrate was concentrated under reduced pressure to afford tert-butyl 3-(((R)-1-(tert-butoxycarbonyl)pyrrolidin-2-yl)((methylsulfonyl)oxy)methyl)-2-oxopyrrolidine-1-carboxylate (14.1 g, crude) as a brown oil. LCMS (ESI, m/z): [M+H]⁺=463.2.

### Step 3. Synthesis of tert-butyl (R,E)-3-((1-(tert-butoxycarbonyl)pyrrolidin-2-yl)methylene)-2-oxopyrrolidine-1-carboxylate

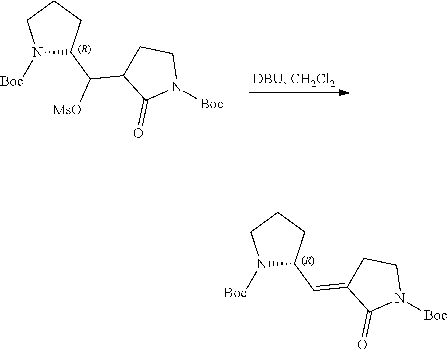

To a solution of tert-butyl 3-(((R)-1-(tert-butoxycarbonyl)pyrrolidin-2-yl)((methylsulfonyl)oxy)methyl)-2-oxopyrrolidine-1-carboxylate (5.0 g, crude) in DCM (50.0 mL) was added DBU (8.2 g, 54.04 mmol) at room temperature. The resulting mixture was stirred at room temperature for 2 h. After the reaction was completed, the mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (40/60, v/v) to afford tert-butyl (R,E)-3-((1-(tert-butoxycarbonyl)pyrrolidin-2-yl)methylene)-2-oxopyrrolidine-1-carboxylate (3.1 g, 78%) as a brown oil. LCMS (ESI, m/z): [M+H]⁺=367.2.

### Step 4. Synthesis of (R,E)-3-(pyrrolidin-2-ylmethylene)pyrrolidin-2-one; 2,2,2-trifluoroacetic acid

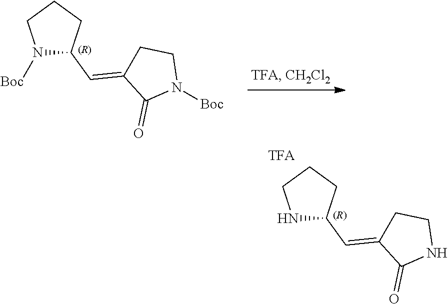

To a solution of tert-butyl (R,E)-3-((1-(tert-butoxycarbonyl)pyrrolidin-2-yl)methylene)-2-oxopyrrolidine-1-carboxylate (1.5 g, 4.09 mmol) in DCM (10.0 mL) was TFA (10.0 mL) at room temperature. The resulting mixture was stirred at room temperature for 2 h. After the reaction was completed, the resulting mixture was concentrated under reduced pressure to afford (R,E)-3-(pyrrolidin-2-ylmethylene)pyrrolidin-2-one; 2,2,2-trifluoroacetic acid (1.0 g, crude) as a yellow solid. LCMS (ESI, m/z): [M+H]⁺=167.1.

### Step 5. Synthesis of (R,E)-3-((1-methylpyrrolidin-2-yl)methylene)pyrrolidin-2-one

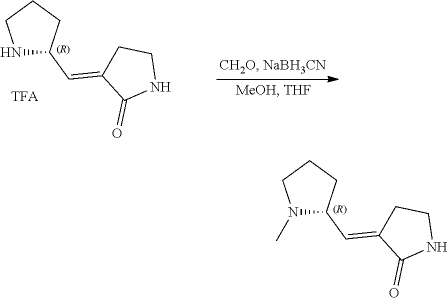

To a solution of (R,E)-3-(pyrrolidin-2-ylmethylene)pyrrolidin-2-one; 2,2,2-trifluoroacetic acid (1.5 g, crude) in THF (25.0 mL) and MeOH (5.0 mL) was added formaldehyde (0.8 g, 40%) at room temperature. The resulting mixture was stirred at room temperature for 1 h. Then NaBH₃CN (22.6 g, 40.61 mmol) was added to the mixture at 0° C. The resulting mixture was stirred at room temperature for 2 h. After the reaction was completed, the resulting mixture was quenched with water and concentrated under reduced pressure. The residue was purified by reverse phase flash chromatography with acetonitrile/water (20/80, v/v) to afford (R,E)-3-((1-methylpyrrolidin-2-yl)methylene)pyrrolidin-2-one (300.0 mg, 17%) as a yellow solid. LCMS (ESI, m/z): [M+H]⁺=181.1.

### Step 6. Synthesis of (R,E)-1-(4-((4-([1,2,4]triazolo[1,5-a]pyridin-7-yloxy)-3-methylphenyl)amino)pyrido[3,2-d]pyrimidin-6-yl)-3-((1-methylpyrrolidin-2-yl)methylene)pyrrolidin-2-one (Compound 90)

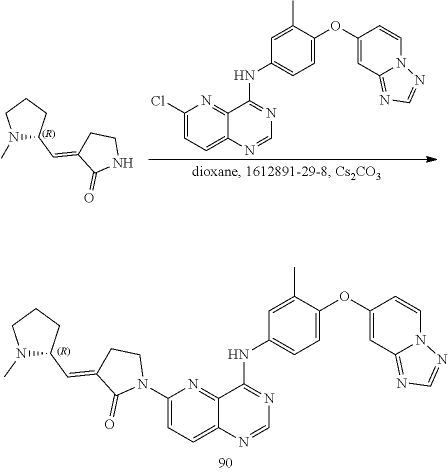

To a solution of (R,E)-3-((1-methylpyrrolidin-2-yl)methylene)pyrrolidin-2-one (178.5 mg, 0.99 mmol) in 1,4-dioxane (10.0 mL) was added Cs₂CO₃ (484.1 mg, 1.49 mmol), Pd-PEPPSI-IPentCl 2-methylpyridine (o-picoline) (84.1 mg, 0.11 mmol) and 6-chloro-N-(3-methyl-4-{[1,2,4]triazolo[1,5-a]pyridin-7-yloxy}phenyl)pyrido[3,2-d]pyrimidin-4-amine (200.0 mg, 0.50 mmol) at room temperature under N₂. The resulting mixture was stirred at 120° C. for 2 h. After the reaction was completed, the mixture was cooled to room temperature and filtered. The filtrate was concentrated under reduced pressure. The residue was purified by flash column chromatography with dichloromethane/methanol (90/10, v/v) and then purified by Prep-HPLC with the following conditions: (Column: XBridge Prep Phenyl OBD Column, 19×250 mm, 5 μm; Mobile Phase A: Water (10 mmol/L NH₄HCO₃), Mobile Phase B: MeOH-HPLC; Flow rate: 20 mL/min; Gradient: 75% B to 75% B in 11 min; Wave Length: 254 nm) to afford (R,E)-1-(4-((4-([1,2,4]triazolo[1,5-a]pyridin-7-yloxy)-3-methylphenyl)amino)pyrido[3,2-d]pyrimidin-6-yl)-3-((1-methylpyrrolidin-2-yl)methylene)pyrrolidin-2-one (Compound 90) (11.2 mg, 4%) as a white solid. LCMS (ESI, m/z): [M+H]⁺=548.2. ¹H NMR (400 MHz, DMSO-d₆): δ 9.61 (s, 1H), 9.01-8.94 (m, 2H), 8.65 (s, 1H), 8.39 (s, 1H), 8.29-8.27 (m, 1H), 8.05-7.97 (m, 2H), 7.26 (d, J=8.4 Hz, 1H), 7.05-7.03 (m, 1H), 6.81 (d, J=2.4 Hz, 1H), 6.48-6.39 (m, 1H), 4.38-4.35 (m, 2H), 3.08-3.04 (m, 1H), 2.93-2.90 (m, 3H), 2.26-2.22 (m, 7H), 2.08-2.04 (s, 1H), 1.88-1.82 (m, 2H), 1.59-1.51 (m, 1H).

### Example S84: Synthesis of (3E)-3-[2-(dimethylamino)ethylidene]-1-[4-({3-fluoro-4-[(1-methyl-1,3-benzodiazol-5 yl)oxy]phenyl}amino)pyrido[3,2-d]pyrimidin-6-yl]pyrrolidin-2-one (Compound 91)

### Step 1. Synthesis of 5-(2-fluoro-4-nitrophenoxy)-1-methyl-1,3-benzodiazole

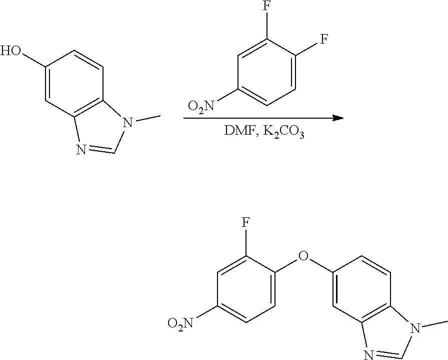 [SMILES: Cn1cnc2cc(O)ccc21 ; O=[N+]([O-])c1ccc(F)c(F)c1 ; Cn1cnc2cc(Oc3ccc([N+](=O)[O-])cc3F)ccc21]

A mixture of 1-methyl-1,3-benzodiazol-5-ol (1.0 g, 6.74 mmol), K₂CO₃ (2.8 g, 20.24 mmol) and 1,2-difluoro-4-nitrobenzene (1.1 g, 6.74 mmol) in DMF (20.0 mL) was stirred at 50° C. for 16 h. After the reaction was completed, the resulting mixture was diluted with H₂O and extracted with ethyl acetate. The combined organic layers were washed with brine, dried over anhydrous sodium sulfate and filtered. The filtrate was concentrated under vacuum. The residue was purified by flash column chromatography with petroleum ether/ethyl acetate (65/35, v/v) to afford 5-(2-fluoro-4-nitrophenoxy)-1-methyl-1,3-benzodiazole (1.7 g, 87%) as a yellow solid. LCMS (ESI, m/z): [M+H]⁺=288.1.

### Step 2. Synthesis of 3-fluoro-4-[(1-methyl-1,3-benzodiazol-5-yl)oxy]aniline

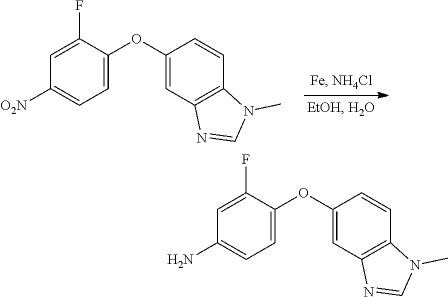

A mixture of 5-(2-fluoro-4-nitrophenoxy)-1-methyl-1,3-benzodiazole (2.0 g, 6.96 mmol), Fe (3.9 g, 69.63 mmol) and NH₄Cl (3.7 g, 69.63 mmol) in CH₃OH (50.0 mL) and H₂O (10.0 mL) was stirred at 80° C. for 1 h. After the reaction was completed, the resulting mixture was filtered. The filtrate was concentrated under reduced pressure. The residue was purified by flash column chromatography with CH₂Cl₂/CH₃OH (90/10, v/v) to afford 3-fluoro-4-[(1-methyl-1,3-benzodiazol-5-yl)oxy]aniline (1.5 g, 83%) as a yellow solid. LCMS (ESI, m/z): [M+H]⁺=258.1.

### Step 3. Synthesis of 6-chloro-N-{3-fluoro-4-[(1-methyl-1,3-benzodiazol-5-yl)oxy]phenyl}pyrido[3,2-d]pyrimidin-4-amine

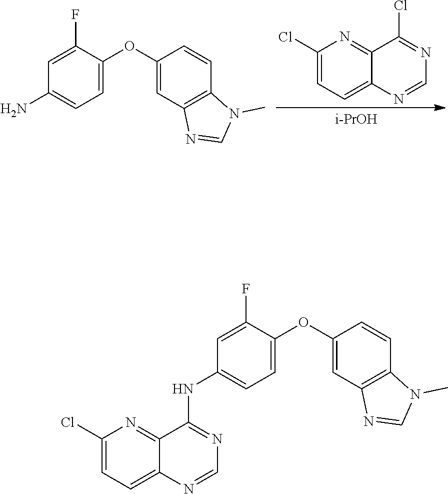 [SMILES: "i-PrOH" ; Cn1cnc2cc(Oc3ccc(N)cc3F)ccc21 ; Clc1ccc2ncnc(Cl)c2n1 ; Cn1cnc2cc(Oc3ccc(Nc4ncnc5ccc(Cl)nc45)cc3F)ccc21]

A mixture of 3-fluoro-4-[(1-methyl-1,3-benzodiazol-5-yl)oxy]aniline (500.0 mg, 1.94 mmol) and 4,6-dichloropyrido[3,2-d]pyrimidine (388.7 mg, 1.94 mmol) in i-PrOH (10.0 mL) was stirred at room temperature for 3 h. After the reaction was completed, the resulting mixture was concentrated under vacuum. The residue was purified by flash column chromatography with CH₂Cl₂/CH₃OH (84/16, v/v) to afford 6-chloro-N-{3-fluoro-4-[(1-methyl-1,3-benzodiazol-5-yl)oxy]phenyl}pyrido[3,2-d]pyrimidin-4-amine (800.0 mg, 97%) as a yellow solid. LCMS (ESI, m/z): [M+H]⁺=421.1.

### Step 4. Synthesis of (3E)-3-[2-(dimethylamino)ethylidene]-1-[4-({3-fluoro-4-[(1-methyl-1,3-benzodiazol-5-yl)oxy]phenyl}amino)pyrido[3,2-d]pyrimidin-6-yl]pyrrolidin-2-one (Compound 91)

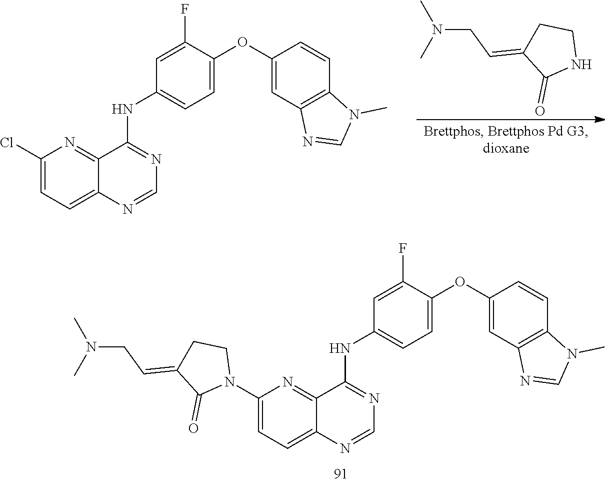

A mixture of 6-chloro-N-{3-fluoro-4-[(1-methyl-1,3-benzodiazol-5-yl)oxy]phenyl}pyrido[3,2-d]pyrimidin-4-amine (163.7 mg, 0.39 mmol), (3E)-3-[2-(dimethylamino)ethylidene]pyrrolidin-2-one (60.0 mg, 0.39 mmol), BrettPhos (41.7 mg, 0.08 mmol), Cs₂CO₃ (380.3 mg, 1.17 mmol) and BrettPhos Pd G3 (35.2 mg, 0.04 mmol) in dioxane (10.0 mL) was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (74/26, v/v) and then purified by Prep-HPLC with the following conditions (Column: Xselect CSH C18 OBD Column 30×150 mm, 511 m; Mobile Phase A: Water (0.1% FA), Mobile Phase B: ACN; Flow rate: 60 mL/min; Gradient: 9% B to 20% B in 12 min, 220 nm) to afford (3E)-3-[2-(dimethylamino)ethylidene]-1-[4-({3-fluoro-4-[(1-methyl-1,3-benzodiazol-5-yl)oxy]phenyl}amino)pyrido[3,2-d]pyrimidin-6-yl]pyrrolidin-2-one (Compound 91) (11.5 mg, 6%) as a yellow solid. LCMS (ESI, m/z): [M+H]⁺=539.3. ¹H NMR (400 MHz, DMSO-d₆): δ 9.65 (s, 1H), 8.99 (d, J=9.2 Hz, 1H), 8.66 (s, 1H), 8.28 (d, J=9.2 Hz, 1H), 8.20-8.15 (m, 2H), 7.77 (d, J=8.8 Hz, 1H), 7.60 (d, J=8.8 Hz, 1H), 7.23-7.16 (m, 2H), 7.09-7.06 (m, 1H), 6.60-6.56 (m, 1H), 4.37-4.34 (m, 2H), 3.86 (s, 3H), 3.15-3.11 (m, 2H), 2.89-2.85 (m, 2H), 2.21 (s, 6H).

### Example S85: Synthesis of (3E)-3 [2-(dimethylamino)ethylidene]-1-[4-({4-[(1-methyl-1,3-benzodiazol-5 yl)oxy]phenyl}amino)pyrido[3,2-d]pyrimidin-6-yl]pyrrolidin-2-one (Compound 92)

### Step 1. Synthesis of 1-methyl-5-(4-nitrophenoxy)-1,3-benzodiazole

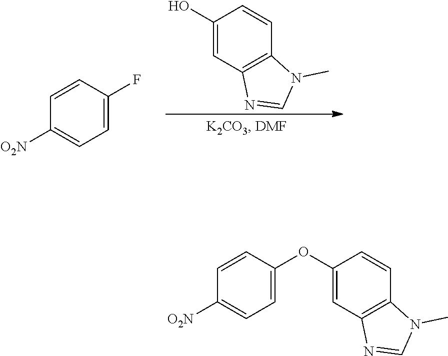 [SMILES: O=[N+]([O-])c1ccc(F)cc1 ; Cn1cnc2cc(O)ccc21 ; Cn1cnc2cc(Oc3ccc([N+](=O)[O-])cc3)ccc21]

A mixture of 4-fluoronitrobenzene (1.0 g, 7.08 mmol), 1-methyl-1,3-benzodiazol-5-ol (1.1 g, 7.08 mmol) and K₂CO₃ (2.9 g, 21.26 mmol) in DMF (50.0 mL) was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (65/35, v/v) to afford 1-methyl-5-(4-nitrophenoxy)-1,3-benzodiazole (1.8 g, 94%) as a yellow solid. LCMS (ESI, m/z): [M+H]⁺=270.1.

### Step 2. Synthesis of 4-[(1-methyl-1,3-benzodiazol-5-yl)oxy]aniline

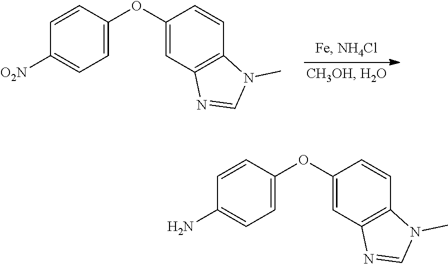 [SMILES: Cn1cnc2cc(Oc3ccc([N+](=O)[O-])cc3)ccc21 ; Cn1cnc2cc(Oc3ccc(N)cc3)ccc21]

A mixture of 1-methyl-5-(4-nitrophenoxy)-1,3-benzodiazole (1.8 g, 6.68 mmol), Fe (3.7 g, 66.85 mmol) and NH₄Cl (3.5 g, 66.85 mmol) in CH₃OH (50.0 mL) and H₂O (10.0 mL) was stirred at 80° C. for 1 h. After the reaction was completed, the resulting mixture was filtered. The filtrate was concentrated under reduced pressure. The residue was purified by flash column chromatography with CH₂Cl₂/CH₃OH (90/10, v/v) to afford 4-[(1-methyl-1,3-benzodiazol-5-yl)oxy]aniline (1.5 g, 93%) as a yellow oil. LCMS (ESI, m/z): [M+H]⁺=240.1.

### Step 3. Synthesis of 6-chloro-N-{4-[(1-methyl-1,3-benzodiazol-5-yl)oxy]phenyl}pyrido[3,2-d]pyrimidin-4-amine

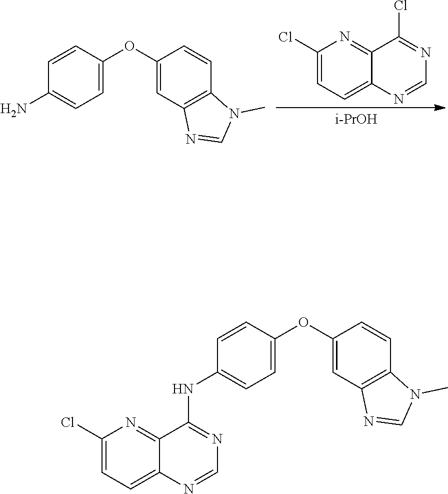 [SMILES: "i-PrOH" ; Cn1cnc2cc(Oc3ccc(N)cc3)ccc21 ; Clc1ccc2ncnc(Cl)c2n1 ; Cn1cnc2cc(Oc3ccc(Nc4ncnc5ccc(Cl)nc45)cc3)ccc21]

A mixture of 4-[(1-methyl-1,3-benzodiazol-5-yl)oxy]aniline (500.0 mg, 2.09 mmol) and 4,6-dichloropyrido[3,2-d]pyrimidine (417.9 mg, 2.09 mmol) in i-PrOH (10.0 mL) was stirred at 90° C. for 1 h. After the reaction was completed, the resulting mixture was concentrated under vacuum. The residue was purified by flash column chromatography with CH₂Cl₂/CH₃OH (91/9, v/v) to afford 6-chloro-N-{4-[(1-methyl-1,3-benzodiazol-5-yl)oxy]phenyl}pyrido[3,2-d]pyrimidin-4-amine (800.0 mg, 95%) as a yellow solid. LCMS (ESI, m/z): [M+H]⁺=403.1.

### Step 4. Synthesis of (3E)-3-[2-(dimethylamino)ethylidene]-1-[4-({4-[(1-methyl-1,3-benzodiazol-5-yl)oxy]phenyl}amino)pyrido[3,2-d]pyrimidin-6-yl]pyrrolidin-2-one (Compound 92)

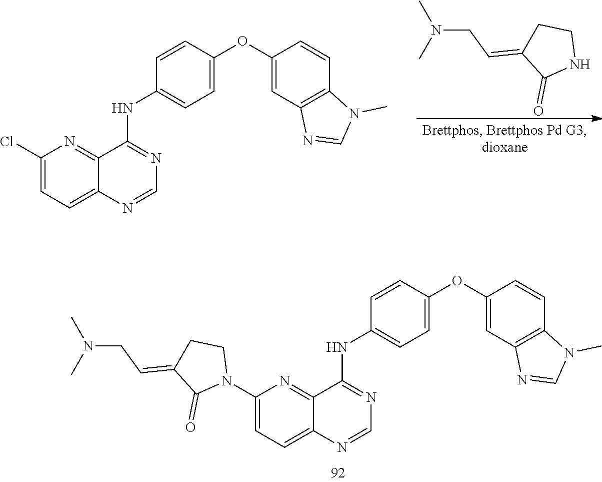

A mixture of 6-chloro-N-{4-[(1-methyl-1,3-benzodiazol-5-yl)oxy]phenyl}pyrido[3,2-d]pyrimidin-4-amine (156.7 mg, 0.38 mmol), (3E)-3-[2-(dimethylamino)ethylidene]pyrrolidin-2-one (60.0 mg, 0.38 mmol), Cs₂CO₃ (380.3 mg, 1.16 mmol), BrettPhos (41.7 mg, 0.07 mmol) and BrettPhos Pd G3 (35.2 mg, 0.03 mmol) in dioxane (10.0 mL) was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (75/25, v/v) and then purified by prep-HPLC with the following conditions (Column: YMC-Actus Triart C18 ExRS, 30×150 mm, 5 μm; Mobile Phase A: Water (10 mmol/L NH₄HCO₃), Mobile Phase B: ACN; Flow rate: 60 mL/min; Gradient: 32% B to 43% B in 10 min, 254 nm) to afford (3E)-3-[2-(dimethylamino)ethylidene]-1-[4-({4-[(1-methyl-1,3-benzodiazol-5-yl)oxy]phenyl}amino)pyrido[3,2-d]pyrimidin-6-yl]pyrrolidin-2-one (Compound 92) (12.0 mg, 5%) as a yellow solid. LCMS (ESI, m/z): [M+H]⁺=521.4. ¹H NMR (400 MHz, DMSO-d₆): δ 9.56 (s, 1H), 8.95 (d, J=9.2 Hz, 1H), 8.56 (s, 1H), 8.24-8.21 (m, 2H), 7.88 (d, J=9.2 Hz, 2H), 7.61 (d, J=8.8 Hz, 1H), 7.29 (d, J=2.0 Hz, 1H), 7.08-7.03 (m, 3H), 6.57-6.54 (m, 1H), 4.34-4.30 (m, 2H), 3.86 (s, 3H), 3.10 (d, J=6.8 Hz, 2H), 2.87-2.83 (m, 2H), 2.20 (s, 6H).

### Example S86: Synthesis of (3E,4S)-3-[2-(dimethylamino)ethylidene]-4-methyl-1-[4-({3-methyl-4-[(1-methyl-1,2,3-benzotriazol-5-yl)oxy]phenyl}amino)pyrido[3,4-d]pyrimidin-6-yl]pyrrolidin-2-one and (3E,4R)-3-[2-(dimethylamino)ethylidene]-4-methyl-1-[4-({3-methyl-4-[(1-methyl-1,2,3-benzotriazol-5-yl)oxy]phenyl}amino)pyrido[3,4-d]pyrimidin-6-yl]pyrrolidin-2-one (Compounds 93 and 94)

### Step 1. Synthesis of 6-chloro-N-{3-methyl-4-[(1-methyl-1,2,3-benzotriazol-5-yl)oxy]phenyl}pyrido[3,4-d]pyrimidin-4-amine

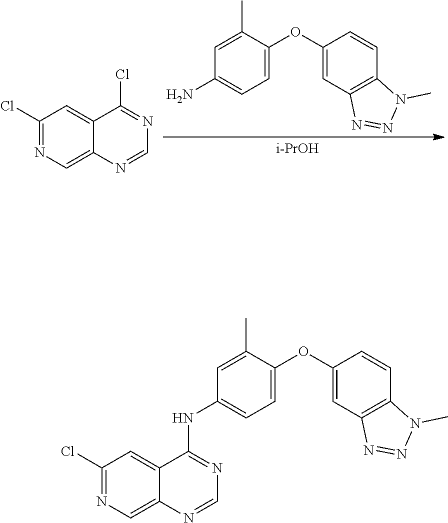 [SMILES: "i-PrOH" ; Clc1cc2c(Cl)ncnc2cn1 ; Cc1cc(N)ccc1Oc1ccc2c(c1)nnn2C ; Cc1cc(Nc2ncnc3cnc(Cl)cc23)ccc1Oc1ccc2c(c1)nnn2C]

To a solution of 4,6-dichloropyrido[3,4-d]pyrimidine (150.0 mg, 0.75 mmol) in i-PrOH (5.0 mL) was added 3-methyl-4-[(1-methyl-1,2,3-benzotriazol-5-yl)oxy]aniline (190.7 mg, 0.75 mmol) at 0° C. The resulting mixture was stirred at room temperature for 2 h. After the reaction was completed, the resulting mixture was concentrated under reduced pressure. The residue was purified by flash column chromatography with CH₂Cl₂/MeOH (10/1, v/v) to afford 6-chloro-N-{3-methyl-4-[(1-methyl-1,2,3-benzotriazol-5-yl)oxy]phenyl}pyrido[3,4-d]pyrimidin-4-amine (150.0 mg, 48%) as a yellow solid. LCMS (ESI, m/z): [M+H]⁺=418.1.

### Step 2. Synthesis of (3E)-3-[2-(dimethylamino)ethylidene]-4-methyl-1-[4-({3-methyl-4-[(1-methyl-1,2,3-benzotriazol-5-yl)oxy]phenyl}amino)pyrido[3,4-d]pyrimidin-6-yl]pyrrolidin-2-one

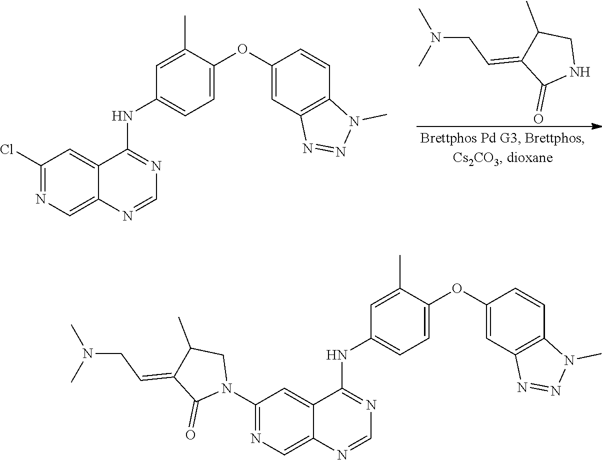

To a solution of 6-chloro-N-{3-methyl-4-[(1-methyl-1,2,3-benzotriazol-5-yl)oxy]phenyl}pyrido[3,4-d]pyrimidin-4-amine (130.0 mg, 0.31 mmol) in dioxane (3.0 mL) were added (3E)-3-[2-(dimethylamino)ethylidene]-4-methylpyrrolidin-2-one (52.3 mg, 0.31 mmol), Cs₂CO₃ (202.7 mg, 0.62 mmol), BrettPhos Pd G3 (28.2 mg, 0.03 mmol) and BrettPhos (33.4 mg, 0.06 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford (3E)-3-[2-(dimethylamino)ethylidene]-4-methyl-1-[4-({3-methyl-4-[(1-methyl-1,2,3-benzotriazol-5-yl)oxy]phenyl}amino)pyrido[3,4-d]pyrimidin-6-yl]pyrrolidin-2-one (50.0 mg, 29%) as a yellow solid. LCMS (ESI, m/z): [M+H]⁺=550.3.

### Step 3. Separation of (3E,4S)-3-[2-(dimethylamino)ethylidene]-4-methyl-1-[4-({3-methyl-4-[(1-methyl-1,2,3-benzotriazol-5-yl)oxy]phenyl}amino)pyrido[3,4-d]pyrimidin-6-yl]pyrrolidin-2-one and (3E,4R)-3-[2-(dimethylamino)ethylidene]-4-methyl-1-[4-({3-methyl-4-[(1-methyl-1,2,3-benzotriazol-5-yl)oxy]phenyl}amino)pyrido[3,4-d]pyrimidin-6-yl]pyrrolidin-2-one (Compounds 93 and 94)

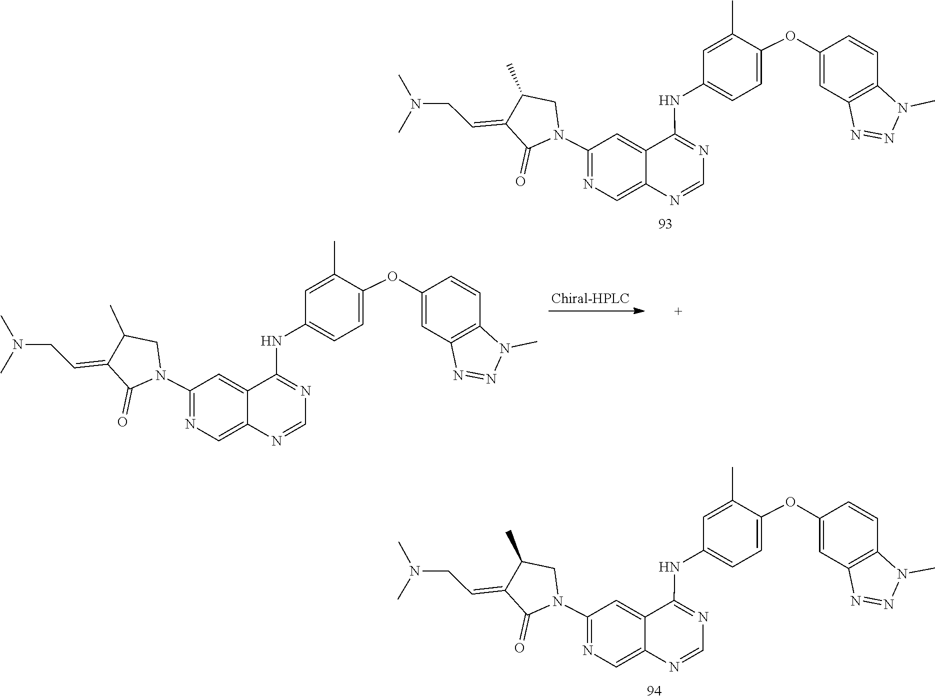

The racemic product of (3E)-3-[2-(dimethylamino)ethylidene]-4-methyl-1-[4-({3-methyl-4-[(1-methyl-1,2,3-benzotriazol-5-yl)oxy]phenyl}amino)pyrido[3,4-d]pyrimidin-6-yl]pyrrolidin-2-one (50.0 mg, 0.09 mmol) was separated by Prep-Chiral-HPLC with the following conditions: (Column: CHIRAL ART Amylose-SA, 2×25 cm, 5 μm; Mobile Phase A: Hex (0.5% 2M NH₃-MeOH)-HPLC, Mobile Phase B: MeOH: DCM=1: 1-HPLC; Flow rate: 20 mL/min; Gradient: 30% B to 30% B in 14 min; Wave Length: 220/254 nm; RT1(min): 11.06; RT2(min): 13.08) to afford (3E,4S)-3-[2-(dimethylamino)ethylidene]-4-methyl-1-[4-({3-methyl-4-[(1-methyl-1,2,3-benzotriazol-5-yl)oxy]phenyl}amino)pyrido[3,4-d]pyrimidin-6-yl]pyrrolidin-2-one Enantiomer 1 (assumed, 16.1 mg, 64%) as a yellow solid and (3E,4R)-3-[2-(dimethylamino)ethylidene]-4-methyl-1-[4-({3-methyl-4-[(1-methyl-1,2,3-benzotriazol-5-yl)oxy]phenyl}amino)pyrido[3,4-d]pyrimidin-6-yl]pyrrolidin-2-one Enantiomer 2 (assumed, 14.7 mg, 58%) as a yellow solid. The absolute stereochemistry of Enantiomers 1 and 2 was not assigned. The two enantiomeric structures that could be obtained from chiral separation of the enantiomeric mixture as described above are shown as Compounds 93 and 94 in Table 1.

(3E)-3-[2-(dimethylamino)ethylidene]-4-methyl-1-[4-({3-methyl-4-[(1-methyl-1,2,3-benzotriazol-5-yl)oxy]phenyl}amino)pyrido[3,4-d]pyrimidin-6-yl]pyrrolidin-2-one Enantiomer 1: RT1(min): 11.06; LCMS (ESI, m/z): [M+H]⁺=550.4. 1H NMR (400 MHz, DMSO-d₆): δ 10.30 (s, 1H), 9.19 (s, 1H), 9.08 (s, 1H), 8.63 (s, 1H), 7.90 (d, J=9.2 Hz, 1H), 7.80 (s, 1H), 7.72-7.70 (m, 1H), 7.38-7.36 (m, 1H), 7.30 (d, J=1.6 Hz, 1H), 7.01 (d, J=8.8 Hz, 1H), 6.57-6.54 (m, 1H), 4.32-4.26 (m, 4H), 3.85-3.82 (m, 1H), 3.67-3.52 (m, 3H), 2.55 (s, 6H), 2.26 (s, 3H), 1.26 (d, J=7.2 Hz, 3H).

(3E)-3-[2-(dimethylamino)ethylidene]-4-methyl-1-[4-({3-methyl-4-[(1-methyl-1,2,3-benzotriazol-5-yl)oxy]phenyl}amino)pyrido[3,4-d]pyrimidin-6-yl]pyrrolidin-2-one Enantiomer 2: RT2(min): 13.08; LCMS (ESI, m/z): [M+H]⁺=550.2. 1H NMR (400 MHz, DMSO-d₆): δ 10.28 (s, 1H), 9.19 (s, 1H), 9.07 (s, 1H), 8.62 (s, 1H), 7.90 (d, J=8.8 Hz, 1H), 7.81 (d, J=2.4 Hz, 1H), 7.73-7.70 (m, 1H), 7.38-7.35 (m, 1H), 7.31 (d, J=2.0 Hz, 1H), 7.00 (d, J=8.4 Hz, 1H), 6.55-6.51 (m, 1H), 4.31 (s, 3H), 4.29-4.23 (m, 1H), 3.82-3.79 (m, 1H), 3.29-3.23 (m, 2H), 3.18-3.12 (m, 1H), 2.25-2.18 (m, 9H), 1.25 (d, J=6.8 Hz, 3H).

### Example S87: Synthesis of 1-(4-((4-([1,2,4]triazolo[1,5-a]pyridin-7-yloxy)-3-methylphenyl)amino)pyrido[3,2-d]pyrimidin-6-yl)-3-methylenepiperidin-2-one (Compound 95)

### Step 1. Synthesis of tert-butyl 3-methylene-2-oxopiperidine-1-carboxylate

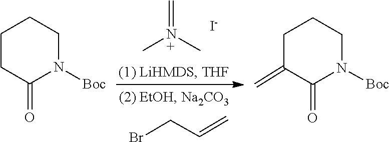

To a solution of tert-butyl 2-oxopiperidine-1-carboxylate (10.0 g, 50.18 mmol) in THF (300.0 mL) was added LiHMDS (100.2 mL, 1.0 mol/L) at −78° C. under N₂. The mixture was stirred at −78° C. for 50 min. Then N-methyl-N-methylenemethanaminium iodide (12.1 g, 65.24 mmol) was added to the mixture. The mixture was stirred at −78° C. for 1.5 h under N₂. After the reaction was completed, the mixture was quenched with saturated NH₄Cl (aq.) and extracted with EtOAc. The combined organic layer was washed with brine, dried over anhydrous sodium sulfate and filtered. The filtrate was concentrated under reduced pressure. The residue was dissolved in EtOH (400.0 mL). Allyl bromide (48.8 g, 401.50 mmol) and Na₂CO₃ (31.9 g, 301.12 mmol) were added to the mixture at room temperature. The final resulting mixture was stirred at room temperature for 2 days. After the reaction was completed, the mixture was filtered. The filtrate was concentrated under reduced pressure. The residue was purified by flash chromatography with petroleum ether/ethyl acetate (70/30, v/v) to afford tert-butyl 3-methylene-2-oxopiperidine-1-carboxylate (7.4 g, 70%) as a yellow oil. LCMS (ESI, m/z): [M+H]⁺=212.1.

### Step 2. Synthesis of 3-methylenepiperidin-2-one

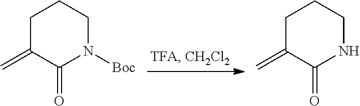

To a solution of tert-butyl 3-methylene-2-oxopiperidine-1-carboxylate (550.0 mg, 2.06 mmol) in CH₂Cl₂ (7.0 mL) was added TFA (5.0 mL) at room temperature. The resulting mixture was stirred at room temperature for 4 h. After the reaction was completed, the resulting mixture was concentrated under vacuum. The pH value of the mixture was adjusted to 7.0 with saturated NaHCO₃ (aq.). The mixture was extracted with CH₂Cl₂. The combined organic layer was washed with brine, dried over anhydrous sodium sulfate and filtered. The filtrate was concentrated under reduced pressure. The residue was purified by flash chromatography with dichloromethane/methanol (92/8, v/v) to afford 3-methylenepiperidin-2-one (210.0 mg, 72%) as a yellow oil. LCMS (ESI, m/z): [M+H]⁺=112.1.

### Step 3. Synthesis of 1-(4-((4-([1,2,4]triazolo[1,5-a]pyridin-7-yloxy)-3-methylphenyl)amino)pyrido[3,2-d]pyrimidin-6-yl)-3-methylenepiperidin-2-one (Compound 95)

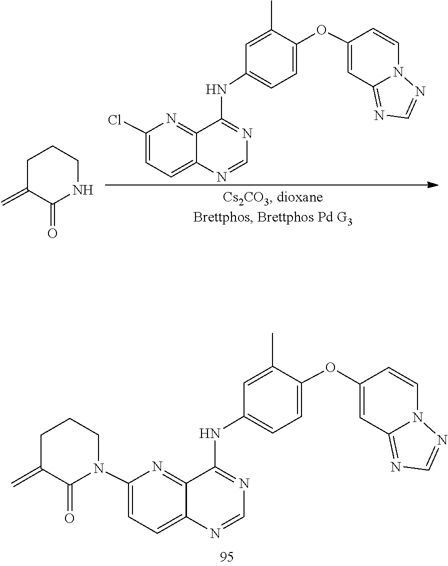

To a solution of 6-chloro-N-(3-methyl-4-{[1,2,4]triazolo[1,5-a]pyridin-7-yloxy}phenyl)pyrido[3,2-d]pyrimidin-4-amine (200.0 mg, 0.50 mmol) in dioxane (8.0 mL) was added 3-methylidenepiperidin-2-one (165.1 mg, 1.48 mmol), BrettPhos (53.1 mg, 0.09 mmol), BrettPhos Pd G3 (44.9 mg, 0.05 mmol) and Cs₂CO₃ (968.1 mg, 2.97 mmol) at room temperature under N₂. The resulting mixture was stirred at 100° C. for 16 h. After the reaction was completed, the mixture was filtered. The filtrate was concentrated under reduced pressure. The residue was purified by flash column chromatography with CH₂Cl₂/MeOH (85/15, v/v) and then purified by Prep-HPLC with the following conditions: (Column: YMC-Actus Triart C18 ExRS, 30×150 mm, 5 μm; Mobile Phase A: Water (10 mmol/L NH₄HCO₃), Mobile Phase B: ACN; Flow rate: 60 mL/min; Gradient: 35% B to 40% B in 10 min; Wave Length: 254 nm) to afford 1-{4-[(3-methyl-4-{[1,2,4]triazolo[1,5-a]pyridin-7-yloxy}phenyl)amino]pyrido[3,2-d]pyrimidin-6-yl}-3-methylidenepiperidin-2-one (Compound 95) (9.7 mg, 4%) as a white solid. LCMS (ESI, m/z): [M+H]⁺=479.3. ¹H NMR (400 MHz, DMSO-d₆): δ 9.76 (s, 1H), 8.94 (d, J=7.6 Hz, 1H), 8.66 (s, 1H), 8.38 (s, 1H), 8.30 (d, J=9.2 Hz, 1H), 8.18 (d, J=8.8 Hz, 1H), 7.99-7.97 (m, 2H), 7.26-7.24 (m, 1H), 7.05-7.02 (m, 1H), 6.80 (d, J=2.4 Hz, 1H), 6.24 (d, J=1.6 Hz, 1H), 5.56 (d, J=1.6 Hz, 1H), 4.32-4.29 (m, 2H), 2.75-2.71 (m, 2H), 2.22 (s, 3H), 2.07-2.00 (m, 2H).

### Example S88: Synthesis of 1-[4-({3-methyl-4-[(1-methyl-1,3-benzodiazol-5-yl)oxy]phenyl}amino)pyrido[3,2-d]pyrimidin-6-yl]-3-methylidenepiperidin-2-one (Compound 96)

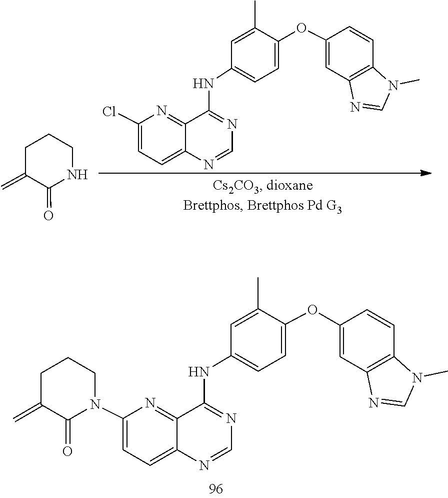

To a solution of 6-chloro-N-{3-methyl-4-[(1-methyl-1,3-benzodiazol-5-yl)oxy]phenyl}pyrido[3,2-d]pyrimidin-4-amine (160.1 mg, 0.38 mmol) in dioxane (10.0 mL) was added 3-methylidenepiperidin-2-one (128.0 mg, 1.15 mmol), Cs₂CO₃ (750.9 mg, 2.30 mmol), BrettPhos (41.2 mg, 0.07 mmol) and BrettPhos Pd G3 (34.8 mg, 0.03 mmol) at room temperature under N₂. The resulting mixture was stirred at 100° C. for 16 h. After the reaction was completed, the mixture was filtered. The filtrate was concentrated under reduced pressure. The residue was purified by flash column chromatography with CH₂Cl₂/MeOH (85/15, v/v) and then purified by Prep-HPLC with the following conditions: (Column: YMC-Actus Triart C18 ExRS, 30×150 mm, 5 μm; Mobile Phase A: Water (10 mmol/L NH₄HCO₃), Mobile Phase B: ACN; Flow rate: 60 mL/min; Gradient: 40% B to 50% B in 10 min; Wave Length: 254 nm) to afford 1-[4-({3-methyl-4-[(1-methyl-1,3-benzodiazol-5-yl)oxy]phenyl}amino)pyrido[3,2-d]pyrimidin-6-yl]-3-methylidenepiperidin-2-one (Compound 96) (24.0 mg, 12%) as a light yellow solid. LCMS (ESI, m/z): [M+H]⁺=492.4. ¹H NMR (400 MHz, DMSO-d₆): δ 9.68 (s, 1H), 8.61 (s, 1H), 8.28 (d, J=9.2 Hz, 1H), 8.17-8.14 (m, 2H), 7.85 (d, J=4.0 Hz, 1H), 7.79-7.76 (m, 1H), 7.57 (d, J=8.8 Hz, 1H), 7.11 (d, J=2.4 Hz, 1H), 7.02-6.99 (m, 1H), 6.90 (d, J=8.0 Hz, 1H), 6.24-6.22 (m, 1H), 5.57-5.55 (m, 1H), 4.31-4.28 (m, 2H), 3.85 (s, 3H), 2.75-2.71 (m, 2H), 2.27 (s, 3H), 2.06-2.00 (m, 2H).

### Example S89: Synthesis of (E)-1-(4-((4-([1,2,4]triazolo[1,5-a]pyridin-7-yloxy)-3-methylphenyl)amino)pyrido[3,2-d]pyrimidin-6-yl)-3-(2-(azetidin-1 yl)ethylidene)pyrrolidin-2-one (Compound 97)

### Step 1. Synthesis of tert-butyl 3-(2-((tert-butyldimethylsilyl)oxy)-1-hydroxyethyl)-2-oxopyrrolidine-1-carboxylate

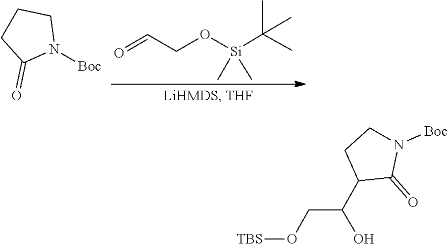

To a solution of tert-butyl 2-oxopyrrolidine-1-carboxylate (25.0 g, 134.97 mmol) in THF (400.0 mL) was added LiHMDS (250.0 mL, 1 mol/L) at −78° C. under N₂. The mixture was stirred at −78° C. for 2 h under N₂. Then a solution of 2-[(tert-butyldimethylsilyl)oxy]acetaldehyde (23.5 g, 134.97 mmol) in THF (50.0 mL) was added dropwise to the mixture at −78° C. The mixture was stirred at −78° C. for additional 2 h under N₂. After the reaction was completed, the reaction mixture was quenched with NH₄Cl (sat.) solution and extracted with ethyl acetate. The combined organic layer was washed with brine, dried over anhydrous sodium sulfate and filtered. The filtrate was concentrated under vacuum The residue was purified by flash column chromatography with petroleum ether/ethyl acetate (3/1, v/v) to afford tert-butyl 3-(2-((tert-butyldimethylsilyl)oxy)-1-hydroxyethyl)-2-oxopyrrolidine-1-carboxylate (15.0 g, 30%) as a yellow oil. LCMS (ESI, m/z): [M+H]⁺=360.2.

### Step 2. Synthesis of tert-butyl 3-(2-((tert-butyldimethylsilyl)oxy)-1-((methylsulfonyl)oxy)ethyl)-2-oxopyrrolidine-1-carboxylate

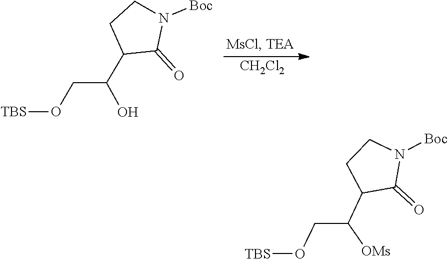

To a solution of tert-butyl 3-(2-((tert-butyldimethylsilyl)oxy)-1-hydroxyethyl)-2-oxopyrrolidine-1-carboxylate (15.0 g, 41.72 mmol) in CH₂Cl₂ (200.0 mL) was added TEA (42.2 g, 417.20 mmol) and MsCl (47.7 g, 417.20 mmol) at 0° C. The resulting mixture was stirred at room temperature for 16 h. After the reaction was completed, the mixture was diluted with H₂O and extracted with CH₂Cl₂. The combined organic layer was washed with brine, dried over anhydrous sodium sulfate and filtered. The filtrate was concentrated under vacuum to afford tert-butyl 3-(2-((tert-butyldimethylsilyl)oxy)-1-((methyl sulfonyl)oxy)ethyl)-2-oxopyrrolidine-1-carboxylate (16.0 g, crude) as a yellow solid. LCMS (ESI, m/z): [M+H]⁺=438.2.

### Step 3. Synthesis of tert-butyl (E)-3-(2-((tert-butyldimethylsilyl)oxy)ethylidene)-2-oxopyrrolidine-1-carboxylate

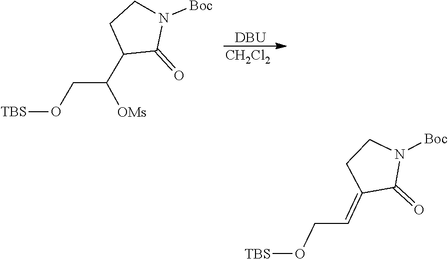

To a solution of tert-butyl 3-(2-((tert-butyldimethylsilyl)oxy)-1-((methylsulfonyl)oxy)ethyl)-2-oxopyrrolidine-1-carboxylate (16.0 g, crude) in CH₂Cl₂ (200.0 mL) was added DBU (8.3 g, 54.84 mmol) at 0° C. The resulting mixture was stirred at room temperature for 2 h. After the reaction was completed, the mixture was diluted with H₂O and extracted with CH₂Cl₂. The combined organic layer was washed with brine, dried over anhydrous sodium sulfate and filtered. The filtrate was concentrated under vacuum. The residue was purified by flash column chromatography with petroleum ether/ethyl acetate (5/1, v/v) to afford tert-butyl (E)-3-(2-((tert-butyldimethylsilyl)oxy)ethylidene)-2-oxopyrrolidine-1-carboxylate (10.0 g, 70%) as a white solid. LCMS (ESI, m/z): [M+H]⁺=342.2.

### Step 4. Synthesis of tert-butyl (E)-3-(2-hydroxyethylidene)-2-oxopyrrolidine-1-carboxylate

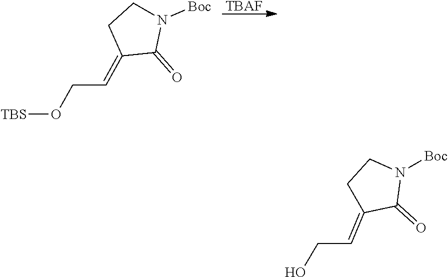

To a solution of tert-butyl (E)-3-(2-((tert-butyldimethylsilyl)oxy)ethylidene)-2-oxopyrrolidine-1-carboxylate (10.0 g, 29.28 mmol) in THE (100.0 mL) was added TBAF (30.0 mL, 1 mol/L) at room temperature. The resulting mixture was stirred at room temperature for 2 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford tert-butyl (E)-3-(2-hydroxyethylidene)-2-oxopyrrolidine-1-carboxylate (6.0 g, 90%) as a white solid. LCMS (ESI, m/z): [M+H]⁺=228.1.

### Step 5. Synthesis of tert-butyl (E)-2-oxo-3-(2-oxoethylidene)pyrrolidine-1-carboxylate

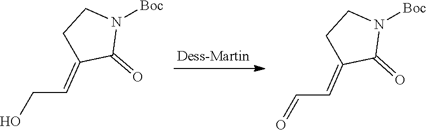 [SMILES: CC(C)(C)OC(=O)N1CC/C(=C\CO)C1=O ; CC(C)(C)OC(=O)N1CC/C(=C\C=O)C1=O]

To a solution of tert-butyl (E)-3-(2-hydroxyethylidene)-2-oxopyrrolidine-1-carboxylate (6.0 g, 26.40 mmol) in CH₂Cl₂ (100.0 mL) was added Dess-Martin (6.0 g, 14.14 mmol) at room temperature under N₂. The resulting mixture was stirred at room temperature for 1 h under N₂. After the reaction was completed, the resulting mixture was concentrated under reduced pressure. The residue was purified by flash column chromatography with petroleum ether/ethyl acetate (1/1, v/v) to afford tert-butyl (E)-2-oxo-3-(2-oxoethylidene)pyrrolidine-1-carboxylate (1.2 g, 20%) as a white solid. LCMS (ESI, m/z): [M+H]⁺=226.1.

### Step 6. Synthesis of tert-butyl (E)-3-(2-(azetidin-1-yl)ethylidene)-2-oxopyrrolidine-1-carboxylate

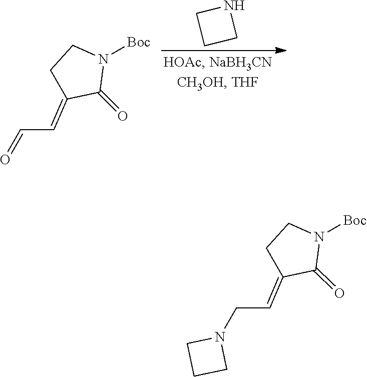

To a solution of azetidine (0.6 g, 10.65 mmol) in THF (15.0 mL)/CH₃OH (3.0 mL) was added HOAc (4.0 mL) and tert-butyl (3E)-2-oxo-3-(2-oxoethylidene)pyrrolidine-1-carboxylate (1.2 g, 5.32 mmol) at room temperature. The resulting mixture was stirred at room temperature for 1 h. Then NaBH₃CN (3.1 g, 47.92 mmol) was added to the mixture at 0° C. The mixture was stirred at room temperature for 1 h. After the reaction was completed, the resulting mixture was concentrated under reduced pressure. The residue was purified by flash column chromatography with CH₂Cl₂/MeOH (10/1, v/v) to afford tert-butyl (3E)-3-[2-(azetidin-1-yl)ethylidene]-2-oxopyrrolidine-1-carboxylate (300.0 mg, 21%) as a white solid. LCMS (ESI, m/z): [M+H]⁺=267.2.

### Step 7. Synthesis of (3E)-3-(2-(azetidin-1-yl)ethylidene)pyrrolidin-2-one

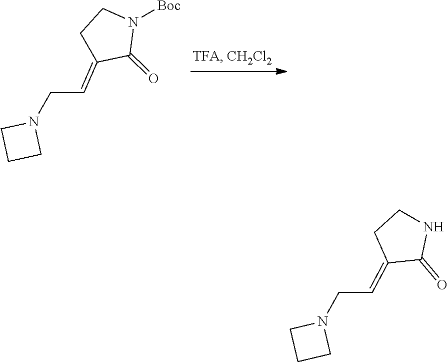 [SMILES: CC(C)(C)OC(=O)N1CC/C(=C\CN2CCC2)C1=O ; O=C1NCC/C1=C\CN1CCC1]

To a solution of tert-butyl (E)-3-(2-(azetidin-1-yl)ethylidene)-2-oxopyrrolidine-1-carboxylate (150.0 mg, 0.56 mmol) in CH₂Cl₂ (4.0 mL) was added TFA (2.0 mL) at room temperature. The mixture was stirred at room temperature for 1 h. After the reaction was completed, the resulting mixture was concentrated under reduced pressure. The residue was purified by reverse phase flash column chromatography with H₂O/ACN (95/5, v/v) to afford (3E)-3-[2-(azetidin-1-yl)ethylidene]pyrrolidin-2-one (45.0 mg, 48%) as a yellow oil. LCMS (ESI, m/z): [M+H]⁺=167.1.

### Step 8. Synthesis of (E)-1-(4-((4-([1,2,4]triazolo[1,5-a]pyridin-7-yloxy)-3-methylphenyl)amino)pyrido[3,2-d]pyrimidin-6-yl)-3-(2-(azetidin-1-yl)ethylidene)pyrrolidin-2-one (Compound 97)

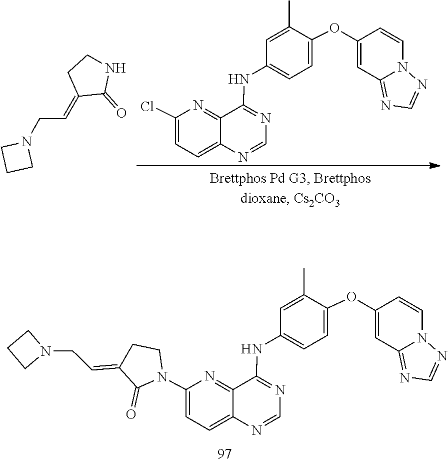

To a solution of (E)-3-(2-(azetidin-1-yl)ethylidene)pyrrolidin-2-one (100.0 mg, 0.60 mmol) in dioxane (10.0 mL) was added N-(4-([1,2,4]triazolo[1,5-a]pyridin-7-yloxy)-3-methylphenyl)-6-chloropyrido[3,2-d]pyrimidin-4-amine (242.9 mg, 0.60 mmol), Cs₂CO₃ (588.0 mg, 1.80 mmol), BrettPhos (64.5 mg, 0.12 mmol) and BrettPhos Pd G3 (54.5 mg, 0.06 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: (Column: Sunfire Prep C18 OBD Column, 19×250 mm, 10 μm; Mobile Phase A: Water (0.1% FA), Mobile Phase B: MeOH-HPLC; Flow rate: 20 mL/min; Gradient: 33% B to 33% B in 18 min; Wave Length: 254 nm) to afford (E)-1-(4-((4-([1,2,4]triazolo[1,5-a]pyridin-7-yloxy)-3-methylphenyl)amino)pyrido[3,2-d]pyrimidin-6-yl)-3-(2-(azetidin-1-yl)ethylidene)pyrrolidin-2-one (4.2 mg, 1%) as a white solid. LCMS (ESI, m/z): [M+H]⁺=534.1. ¹H NMR (400 MHz, DMSO-d₆): δ 9.64 (s, 1H), 8.96-8.85 (m, 2H), 8.62 (s, 1H), 8.39 (s, 1H), 8.27 (d, J=9.2 Hz, 1H), 8.00-7.97 (m, 2H), 7.33 (s, 1H), 7.26 (d, J=8.4 Hz, 1H), 7.05-7.03 (m, 1H), 6.80 (d, J=2.0 Hz, 1H), 4.93-4.88 (m, 2H), 3.21-3.17 (m, 4H), 2.66-2.63 (m, 2H), 2.30-2.27 (m, 2H), 2.22 (s, 3H), 2.02-1.95 (m, 2H).

### Example S90: Synthesis of (E)-1-(4-((3-chloro-4-((1-methyl-1H-benzo[d]imidazol-5-yl)oxy)phenyl)amino)pyrido[3,2-d]pyrimidin-6-yl)-3-(2-(dimethylamino)ethylidene)pyrrolidin-2-one (Compound 98)

### Step 1. Synthesis of 5-(2-chloro-4-nitrophenoxy)-1-methyl-1H-benzo[d]imidazole

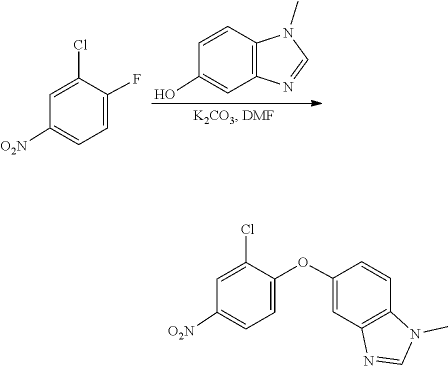

To a solution of 2-chloro-1-fluoro-4-nitrobenzene (3.0 g, 17.09 mmol) in DMF (20.0 mL) was added 1-methyl-1,3-benzodiazol-5-ol (3.0 g, 20.51 mmol) and K₂CO₃ (4.7 g, 34.18 mmol) at room temperature. The resulting mixture was stirred at 100° C. for 3 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (95/5, v/v) to afford 5-(2-chloro-4-nitrophenoxy)-1-methyl-1H-benzo[d]imidazole (4.0 g, 77%) as a yellow solid. LCMS (ESI, m/z): [M+H]⁺=304.0.

### Step 2. Synthesis of 3-chloro-4-((1-methyl-1H-benzo[d]imidazol-5-yl)oxy)aniline

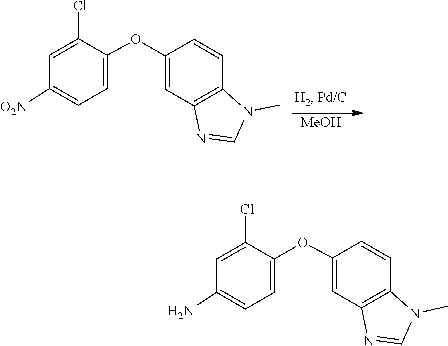 [SMILES: "MeOH" ; Cn1cnc2cc(Oc3ccc([N+](=O)[O-])cc3Cl)ccc21 ; Cn1cnc2cc(Oc3ccc(N)cc3Cl)ccc21]

To a solution of 5-(2-chloro-4-nitrophenoxy)-1-methyl-1,3-benzodiazole (2.0 g, 6.59 mmol) in CH₃OH (20.0 mL) was added Pd/C (800.0 mg, 10% wet) at room temperature under N₂. The resulting mixture was stirred at room temperature for 4 h under H₂. After the reaction was completed, the resulting mixture was filtered. The filtrate was concentrated under reduced pressure. The residue was purified by flash column chromatography CH₂Cl₂/MeOH (90/10, v/v) to afford 3-chloro-4-[(1-methyl-1,3-benzodiazol-5-yl)oxy]aniline (770.0 mg, 93%) as a brown solid. LCMS (ESI, m/z): [M+H]⁺=274.1.

### Step 3. Synthesis of 6-chloro-N-(3-chloro-4-((1-methyl-1H-benzo[d]imidazol-5-yl)oxy)phenyl)pyrido[3,2-d]pyrimidin-4-amine

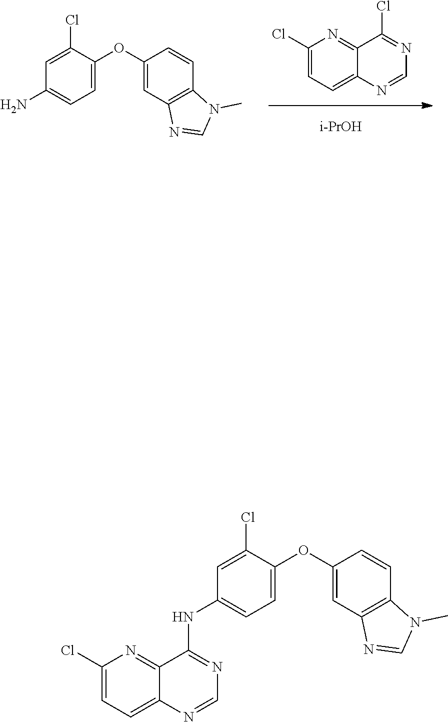 [SMILES: "i-PrOH" ; Cn1cnc2cc(Oc3ccc(N)cc3Cl)ccc21 ; Clc1ccc2ncnc(Cl)c2n1 ; Cn1cnc2cc(Oc3ccc(Nc4ncnc5ccc(Cl)nc45)cc3Cl)ccc21]

To a solution of 3-chloro-4-[(1-methyl-1,3-benzodiazol-5-yl)oxy]aniline (300.0 mg, 1.10 mmol) in i-PrOH (10.0 mL) was added 4,6-dichloropyrido[3,2-d]pyrimidine (220.0 mg, 1.11 mmol) at room temperature. The resulting mixture was stirred at room temperature for 2 h. After the reaction was completed, the resulting mixture was concentrated under reduced pressure. The residue was purified by flash column chromatography with CH₂Cl₂/MeOH (85/15, v/v) to afford 6-chloro-N-{3-chloro-4-[(1-methyl-1,3-benzodiazol-5-yl)oxy]phenyl}pyrido[3,2-d]pyrimidin-4-amine (300.0 mg, 62%) as a yellow solid. LCMS (ESI, m/z): [M+H]⁺=437.2.

### Step 4. Synthesis of (E)-1-(4-((3-chloro-4-((1-methyl-1H-benzo[d]imidazol-5-yl)oxy)phenyl)amino)pyrido[3,2-d]pyrimidin-6-yl)-3-(2-(dimethylamino)ethylidene)pyrrolidin-2-one (Compound 98)

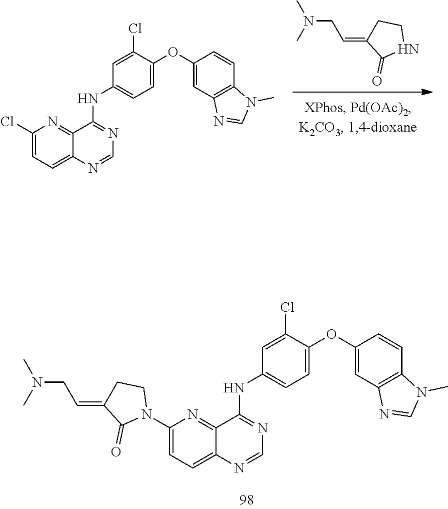

To a solution of 6-chloro-N-{3-chloro-4-[(1-methyl-1,3-benzodiazol-5-yl)oxy]phenyl}pyrido[3,2-d]pyrimidin-4-amine (100.0 mg, 0.23 mmol) in 1,4-dioxane (10.0 mL) was added (E)-3-(2-(dimethylamino)ethylidene)pyrrolidin-2-one (35.3 mg, 0.23 mmol), K₂CO₃ (63.2 mg, 0.46 mmol), XPhos (21.8 mg, 0.05 mmol) and Pd(OAc)₂ (5.1 mg, 0.02 mmol) at room temperature under N₂. The resulting mixture was stirred at 120° C. for 2 h under N₂. After the reaction was completed, the resulting mixture was concentrated under reduced pressure. The residue was purified by flash column chromatography with CH₂Cl₂/MeOH (85/15, v/v) and then purified by Prep-HPLC with the following conditions: (Column: YMC-Actus Triart C18 ExRS, 30×150 mm, 5 μm; Mobile Phase A: Water (10 mmol/L NH₄HCO₃), Mobile Phase B: ACN; Flow rate: 60 mL/min; Gradient: 40% B to 45% B in 10 min; Wave Length: 254 nm) to afford (E)-1-(4-((3-chloro-4-((1-methyl-1H-benzo[d]imidazol-5-yl)oxy)phenyl)amino)pyrido[3,2-d]pyrimidin-6-yl)-3-(2-(dimethylamino)ethylidene)pyrrolidin-2-one (Compound 98) (9.1 mg, 7%) as a white solid. LCMS (ESI, m/z): [M+H]⁺=555.3. ¹H NMR (400 MHz, DMSO-d₆): δ 9.63 (s, 1H), 8.98-8.96 (m, 1H), 8.63 (s, 1H), 8.29-8.24 (m, 2H), 8.21 (s, 1H), 7.92-7.89 (m, 1H), 7.61 (d, J=8.8 Hz, 1H), 7.19 (d, J=2.0 Hz, 1H), 7.10-7.04 (m, 2H), 6.58-6.54 (m, 1H), 4.35-4.31 (m, 2H), 3.86 (s, 3H), 3.10 (d, J=6.8 Hz, 2H), 2.90-2.80 (m, 2H), 2.20 (s, 6H).

### Example S91: Synthesis of (3E)-3-[2-(dimethylamino)ethylidene]-1-[4-({2-fluoro-4-[(1-methyl-1,3-benzodiazol-5-yl)oxy]phenyl}amino)pyrido[3,2-d]pyrimidin-6-yl]pyrrolidin-2-one (Compound 99)

### Step 1. Synthesis of 5-(3-fluoro-4-nitrophenoxy)-1-methyl-1,3-benzodiazole

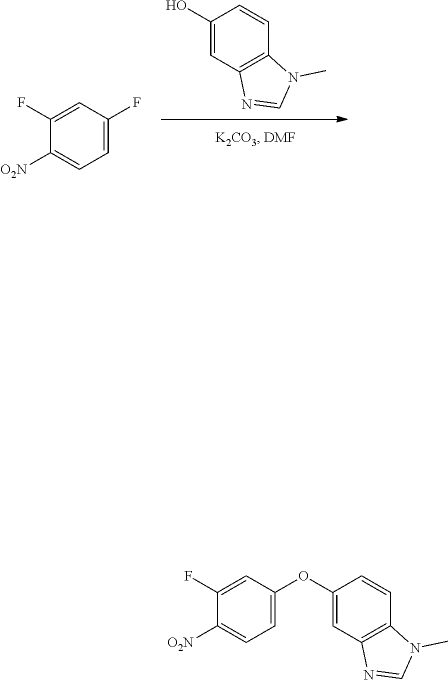

To a solution of 2,4-difluoro-1-nitrobenzene (3.0 g, 17.09 mmol) in DMF (20.0 mL) was added 1-methyl-1,3-benzodiazol-5-ol (3.0 g, 20.51 mmol) and K₂CO₃ (4.7 g, 34.18 mmol) at room temperature. The resulting mixture was stirred at 100° C. for 3 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with ACN/H₂O (80/20, v/v) to afford 5-(3-fluoro-4-nitrophenoxy)-1-methyl-1H-benzo[d]imidazole (500.0 mg, 10%) as a yellow solid. LCMS (ESI, m/z): [M+H]⁺=288.1.

### Step 2. Synthesis of 2-fluoro-4-[(1-methyl-1,3-benzodiazol-5-yl)oxy]aniline

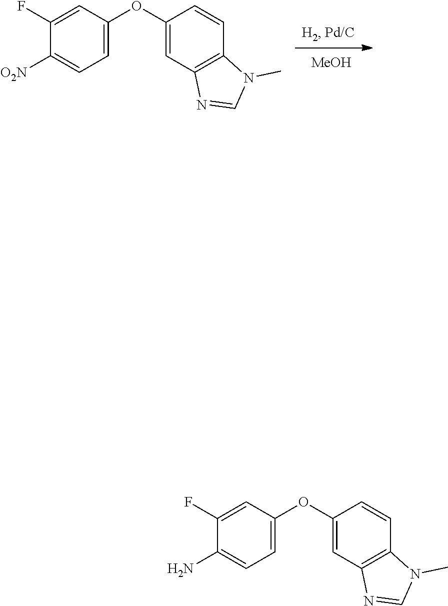 [SMILES: "MeOH" ; Cn1cnc2cc(Oc3ccc([N+](=O)[O-])c(F)c3)ccc21 ; "H2, Pd/C" ; Cn1cnc2cc(Oc3ccc(N)c(F)c3)ccc21]

To a solution of 5-(3-fluoro-4-nitrophenoxy)-1-methyl-1,3-benzodiazole (102.5 mg, 0.35 mmol) in MeOH (20.0 mL) was added Pd/C (26.5 mg, 10% wet) at room temperature under N₂. The resulting mixture was stirred at room temperature for 16 h under H₂. After the reaction was completed, the resulting mixture was filtered. The filtrate was concentrated under reduced pressure to afford 2-fluoro-4-[(1-methyl-1,3-benzodiazol-5-yl)oxy]aniline (115.2 mg, crude) as a yellow solid. LCMS (ESI, m/z): [M+H]⁺=258.0.

### Step 3. Synthesis of 6-chloro-N-{2-fluoro-4-[(1-methyl-1,3-benzodiazol-5-yl)oxy]phenyl}pyrido[3,2-d]pyrimidin-4-amine

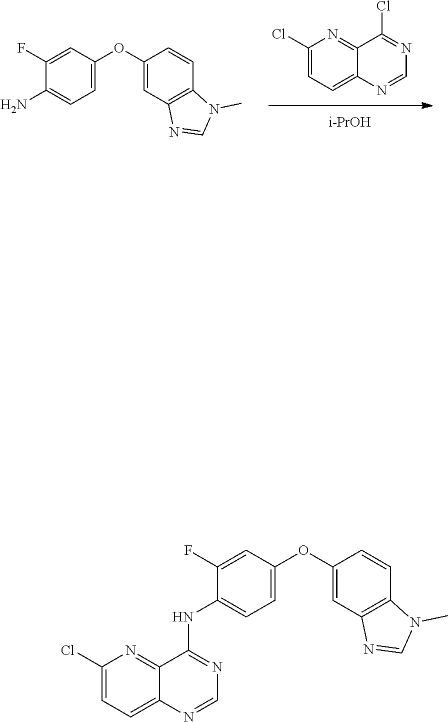 [SMILES: "i-PrOH" ; Cn1cnc2cc(Oc3ccc(N)c(F)c3)ccc21 ; Clc1ccc2ncnc(Cl)c2n1 ; Cn1cnc2cc(Oc3ccc(Nc4ncnc5ccc(Cl)nc45)c(F)c3)ccc21]

To a solution of 2-fluoro-4-[(1-methyl-1,3-benzodiazol-5-yl)oxy]aniline (115.2 mg, 0.44 mmol) in i-PrOH (10.0 mL) was added 4,6-dichloropyrido[3,2-d]pyrimidine (89.5 mg, 0.44 mmol) at room temperature. The resulting mixture was stirred at room temperature for 16 h. After the reaction was completed, the resulting mixture was concentrated under reduced pressure. The residue was purified by flash column chromatography with CH₂Cl₂/MeOH (85/15, v/v) to afford 6-chloro-N-{2-fluoro-4-[(1-methyl-1,3-benzodiazol-5-yl)oxy]phenyl}pyrido[3,2-d]pyrimidin-4-amine (120.0 mg, 63%) as a yellow solid. LCMS (ESI, m/z): [M+H]⁺=421.2.

### Step 4. Synthesis of (3E)-3-[2-(dimethylamino)ethylidene]-1-[4-({2-fluoro-4-[(1-methyl-1,3-benzodiazol-5-yl)oxy]phenyl}amino)pyrido[3,2-d]pyrimidin-6-yl]pyrrolidin-2-one (Compound 99)

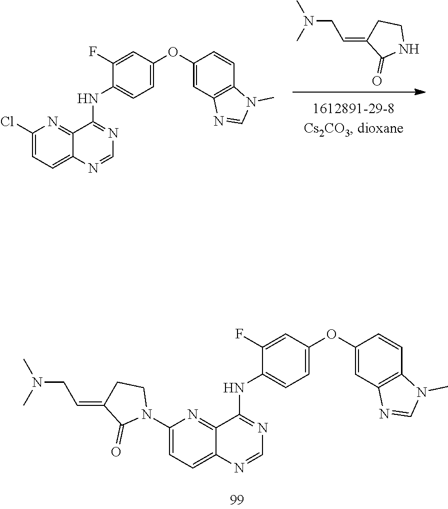

To a solution of 6-chloro-N-{2-fluoro-4-[(1-methyl-1,3-benzodiazol-5-yl)oxy]phenyl}pyrido[3,2-d]pyrimidin-4-amine (110.9 mg, 0.26 mmol) in 1,4-dioxane (6.0 mL) was added (3E)-3-[2-(dimethylamino)ethylidene]pyrrolidin-2-one (44.7 mg, 0.29 mmol), Cs₂CO₃ (233.9 mg, 0.72 mmol) and Pd-PEPPSI-IPentCl 2-methylpyridine (o-picoline) (40.0 mg, 0.04 mmol) at room temperature under N₂. The final reaction mixture was irradiated with microwave radiation at 120° C. for 3 h under N₂. After the reaction was completed, the mixture was cooled to room temperature and filtered. The filtrate was concentrated under reduced pressure. The residue was purified by flash column chromatography with CH₂Cl₂/MeOH (90/10, v/v) and then purified by Prep-HPLC with the following conditions: (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 30% B to 40% B in 8 min; Wave Length: 254 nm) to afford (3E)-3-[2-(dimethylamino)ethylidene]-1-[4-({2-fluoro-4-[(1-methyl-1,3-benzodiazol-5-yl)oxy]phenyl}amino)pyrido[3,2-d]pyrimidin-6-yl]pyrrolidin-2-one (Compound 99) (21.5 mg, 15%) as a yellow solid. LCMS (ESI, m/z): [M+H]⁺=539.3. ¹H NMR (400 MHz, DMSO-d₆): δ 9.55 (s, 1H), 8.98 (d, J=9.2 Hz, 1H), 8.52 (s, 1H), 8.27-8.24 (m, 2H), 7.81-7.77 (m, 1H), 7.66 (d, J=8.8 Hz, 1H), 7.40 (d, J=2.4 Hz, 1H), 7.13-7.11 (m, 1H), 7.00-6.96 (m, 1H), 6.88-6.85 (m, 1H), 6.57-6.55 (m, 1H), 4.29-4.25 (m, 2H), 3.88 (s, 3H), 3.10 (d, J=6.4 Hz, 2H), 2.87-2.84 (m, 2H), 2.20 (s, 6H).

### Example S92: Synthesis of (3E)-1-[4-({3-methyl-4-[(1-methyl-1,3-benzodiazol-5-yl)oxy]phenyl}amino)pyrido[3,2-d]pyrimidin-6-yl]-3-[2-(methylamino)ethylidene]pyrrolidin-2-one; formic acid (Compound 100)

### Step 1. Synthesis of tert-butyl N-methyl-N-{2-[(3E)-2-oxopyrrolidin-3-ylidene]ethyl}carbamate

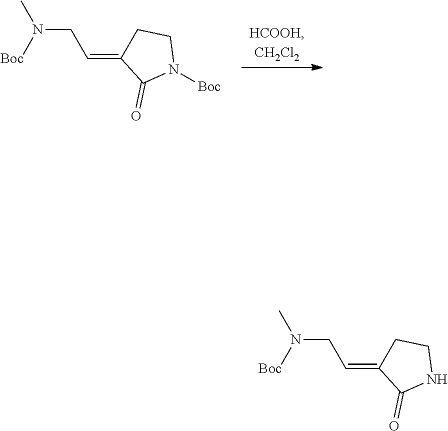

To a solution of tert-butyl (3E)-3-{2-[(tert-butoxycarbonyl)(methyl)amino]ethylidene}-2-oxopyrrolidine-1-carboxylate (500.0 mg, 1.46 mmol) in CH₂Cl₂ (10.0 mL) was added HCOOH (2.0 mL) at room temperature. The mixture was stirred at room temperature for 4 h. After the reaction was completed, the pH value of the mixture was adjusted to 8.0 with saturated NaHCO₃ (aq.). The mixture was extracted with CH₂Cl₂. The combined organic layer was washed with brine, dried over anhydrous sodium sulfate and filtered. The filtrate was concentrated under vacuum. The residue was purified by reverse phase column chromatography with ACN/H₂O (60/40, v/v) afford tert-butyl N-methyl-N-{2-[(3E)-2-oxopyrrolidin-3-ylidene]ethyl}carbamate (260.0 mg, 73%) as a light yellow oil. LCMS (ESI, m/z): [M+H]⁺=241.2.

### Step 2. Synthesis of tert-butyl N-methyl-N-{2-[(3E)-1-[4-({3-methyl-4-[(1-methyl-1,3-benzodiazol-5-yl)oxy]phenyl}amino)pyrido[3,2-d]pyrimidin-6-yl]-2-oxopyrrolidin-3-ylidene]ethyl}carbamate

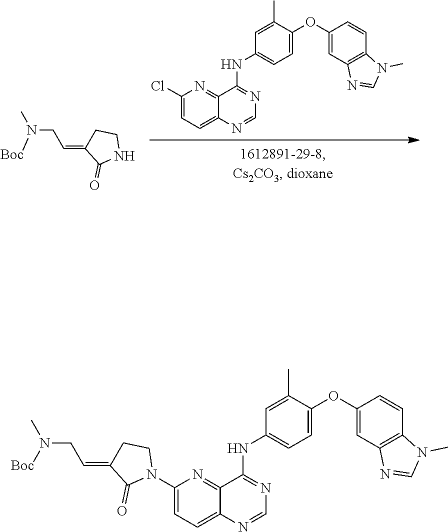

To a solution of tert-butyl N-methyl-N-{2-[(3E)-2-oxopyrrolidin-3-ylidene]ethyl}carbamate (260.0 mg, 1.08 mmol) in 1,4-dioxane (15.0 mL) was added 6-chloro-N-{3-methyl-4-[(1-methyl-1,3-benzodiazol-5-yl)oxy]phenyl}pyrido[3,2-d]pyrimidin-4-amine (541.2 mg, 1.29 mmol), Cs₂CO₃ (1057.5 mg, 3.24 mmol) and Pd-PEPPSI-IPentCl 2-methylpyridine (o-picoline) (90.8 mg, 0.11 mmol) at room temperature under N₂. The resulting mixture was stirred at 100° C. for 16 h. After the reaction was completed, the mixture was cooled to room temperature and filtered. The filtrate was concentrated under reduced pressure. The residue was purified by flash column chromatography with CH₂Cl₂/MeOH (90/10, v/v) to afford tert-butyl N-methyl-N-{2-[(3E)-1-[4-({3-methyl-4-[(1-methyl-1,3-benzodiazol-5-yl)oxy]phenyl}amino)pyrido[3,2-d]pyrimidin-6-yl]-2-oxopyrrolidin-3-ylidene]ethyl}carbamate (210.0 mg, 31%) as a brown oil. LCMS (ESI, m/z): [M+H]⁺=621.3.

### Step 3. Synthesis of (3E)-1-[4-({3-methyl-4-[(1-methyl-1,3-benzodiazol-5-yl)oxy]phenyl}amino)pyrido[3,2-d]pyrimidin-6-yl]-3-[2-(methylamino)ethylidene]pyrrolidin-2-one; formic acid (Compound 100)

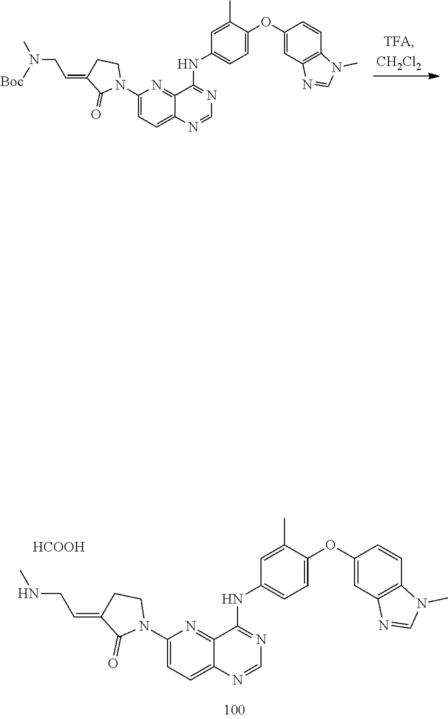

To a solution of tert-butyl N-methyl-N-{2-[(3E)-1-[4-({3-methyl-4-[(1-methyl-1,3-benzodiazol-5-yl)oxy]phenyl}amino)pyrido[3,2-d]pyrimidin-6-yl]-2-oxopyrrolidin-3-ylidene]ethyl}carbamate (110.0 mg, 0.17 mmol) in CH₂Cl₂ (8.0 mL) was added TFA (4.0 mL) at room temperature. The resulting mixture was stirred at room temperature for 2 h. After the reaction was completed, the pH value of the mixture was adjusted to 8.0 with saturated NaHCO₃ (aq.). The mixture was extracted with CH₂Cl₂. The combined organic layer was washed with brine, dried over anhydrous sodium sulfate and filtered. The filtrate was concentrated under vacuum. The residue was purified by flash column chromatography with CH₂Cl₂/MeOH (90/10, v/v) and then purified by Prep-HPLC with the following conditions: (Column: XSelect CSH Fluoro Phenyl, 30×150 mm, 5 μm; Mobile Phase A: ACN, Mobile Phase B: Water (0.1% FA); Flow rate: 60 mL/min; Gradient: 5% B to 10% B in 10 min; Wave Length: 254/220 nm) to afford (3E)-1-[4-({3-methyl-4-[(1-methyl-1,3-benzodiazol-5-yl)oxy]phenyl}amino)pyrido[3,2-d]pyrimidin-6-yl]-3-[2-(methylamino)ethylidene]pyrrolidin-2-one; formic acid (Compound 100) (5.2 mg, 5%) as a yellow solid. LCMS (ESI, m/z): [M+H]⁺=521.4. ¹H NMR (400 MHz, CD₃OD): δ 8.99 (d, J=9.2 Hz, 1H), 8.54 (s, 1H), 8.33 (s, 1H), 8.15-8.12 (m, 2H), 7.79 (d, J=2.4 Hz, 1H), 7.73-7.70 (m, 1H), 7.56 (d, J=8.8 Hz, 1H), 7.15-7.10 (m, 2H), 6.91 (d, J=8.4 Hz, 1H), 6.65-6.62 (s, 1H), 4.37-4.34 (m, 2H), 3.94-3.92 (m, 5H), 3.10-3.03 (m, 2H), 2.82 (s, 3H), 2.32 (s, 3H).

### Example S93: Synthesis of (E)-3-(2-(dimethylamino)ethylidene)-1-(4-((3-methyl-4-((1-methyl-1H-benzo[d][1,2,3]triazol-5-yl)oxy)phenyl)amino)pyrido[3,4-d]pyrimidin-6-yl)pyrrolidin-2-one (Compound 101)

### Step 1. Synthesis of 6-chloro-N-(3-methyl-4-((1-methyl-1H-benzo[d][1,2,3]triazol-5-yl)oxy)phenyl)pyrido[3,4-d]pyrimidin-4-amine

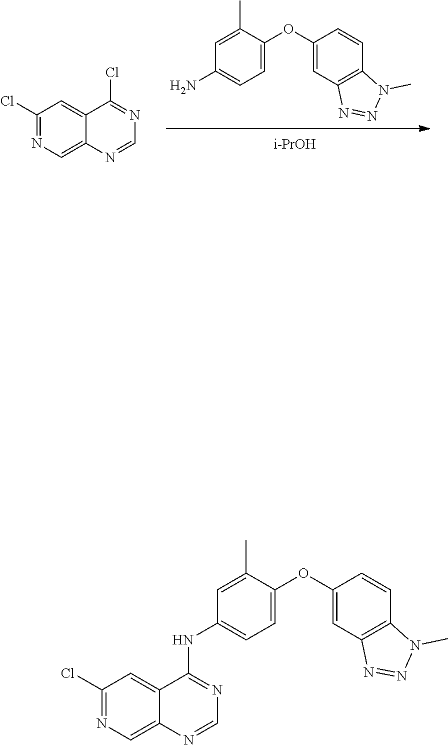 [SMILES: "i-PrOH" ; Clc1cc2c(Cl)ncnc2cn1 ; Cc1cc(N)ccc1Oc1ccc2c(c1)nnn2C ; Cc1cc(Nc2ncnc3cnc(Cl)cc23)ccc1Oc1ccc2c(c1)nnn2C]

To a solution of 3-methyl-4-[(1-methyl-1,2,3-benzotriazol-5-yl)oxy]aniline (279.6 mg, 1.10 mmol) in i-PrOH (10.0 mL) was added 4,6-dichloropyrido[3,4-d]pyrimidine (220.0 mg, 1.10 mmol) at room temperature. The resulting mixture was stirred at room temperature for 4 h. After the reaction was completed, the mixture was concentrated under reduced pressure. The residue was purified by flash column chromatography with CH₂Cl₂/MeOH (90/10, v/v) to afford 6-chloro-N-{3-methyl-4-[(1-methyl-1,2,3-benzotriazol-5-yl)oxy]phenyl}pyrido[3,4-d]pyrimidin-4-amine (225.1 mg, 48%) as a yellow solid. LCMS (ESI, m/z): [M+H]⁺=418.3.

### Step 2. Synthesis of (E)-3-(2-(dimethylamino)ethylidene)-1-(4-((3-methyl-4-((1-methyl-1H-benzo[d][1,2,3]triazol-5-yl)oxy)phenyl)amino)pyrido[3,4-d]pyrimidin-6-yl)pyrrolidin-2-one (Compound 101)

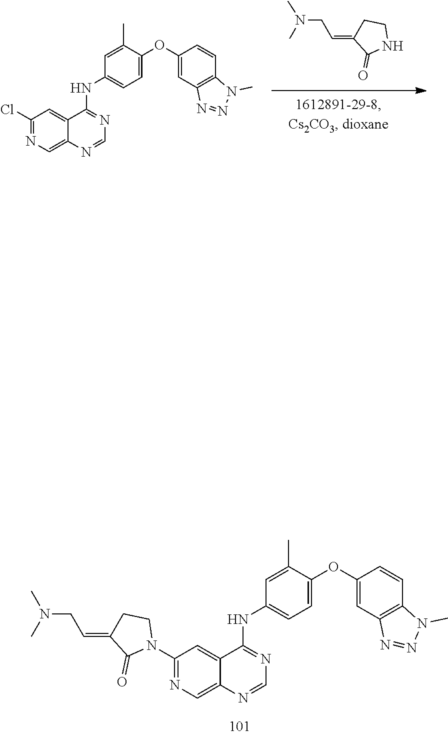

To a solution of 6-chloro-N-{3-methyl-4-[(1-methyl-1,2,3-benzotriazol-5-yl)oxy]phenyl}pyrido[3,4-d]pyrimidin-4-amine (100.0 mg, 0.24 mmol) in 1,4-dioxane (5.0 mL) was added (3E)-3-[2-(dimethylamino)ethylidene]pyrrolidin-2-one (44.2 mg, 0.28 mmol), Cs₂CO₃ (233.9 mg, 0.72 mmol) and Pd-PEPPSI-IPentCl 2-methylpyridine (o-picoline) (40.0 mg, 0.04 mmol) at room temperature under N₂. The final reaction mixture was irradiated with microwave radiation at 100° C. for 2 h under N₂. After the reaction was completed, the mixture was cooled to room temperature and filtered. The filtrate was concentrated under reduced pressure. The residue was purified by flash column chromatography with CH₂Cl₂/MeOH (90/10, v/v) and then purified by Prep-HPLC with the following conditions: (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 32% B to 42% B in 8 min; Wave Length: 254 nm) to afford (3E)-3-[2-(dimethylamino)ethylidene]-1-[4-({3-methyl-4-[(1-methyl-1,2,3-benzotriazol-5-yl)oxy]phenyl}amino)pyrido[3,4-d]pyrimidin-6-yl]pyrrolidin-2-one (Compound 101) (34.6 mg, 26%) as a white solid. LCMS (ESI, m/z): [M+H]⁺=536.3. ¹H NMR (400 MHz, CD₃OD): δ 9.05 (d, J=8.4 Hz, 2H), 8.59 (s, 1H), 7.81-7.79 (m, 2H), 7.72-7.69 (m, 1H), 7.42-7.3-9 (m, 1H), 7.27 (d, J=2.0 Hz, 1H), 7.05 (d, J=8.8 Hz, 1H), 6.76-6.72 (m, 1H), 4.35 (s, 3H), 4.31-4.28 (m, 2H), 3.34-3.32 (m, 2H), 2.97-2.93 (m, 2H), 2.36 (s, 6H), 2.31 (s, 3H).

### Example S94: Synthesis of (E)-3-(2-(bis(methyl-d₃)amino)ethylidene)-1-(4-((3-methyl-4-((1-methyl-1H-benzo[d]imidazol-5 yl)oxy)phenyl)amino)pyrido[3,2-d]pyrimidin-6-yl)pyrrolidin-2-one (Compound 105)

### Step 1. Synthesis of Tert-butyl (3E)-3-[2-(methanesulfonyloxy)ethylidene]-2-oxopyrrolidine-1-carboxylate

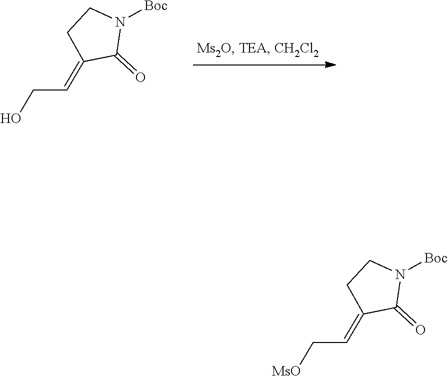 [SMILES: CC(C)(C)OC(=O)N1CC/C(=C\CO)C1=O ; "Ms2O, TEA, CH2Cl2" ; CC(C)(C)OC(=O)N1CC/C(=C\COS(C)(=O)=O)C1=O]

To a solution of tert-butyl (3E)-3-(2-hydroxyethylidene)-2-oxopyrrolidine-1-carboxylate (350.0 mg, 1.54 mmol) in CH₂Cl₂ (10.0 mL) was added TEA (311.7 mg, 3.08 mmol) at room temperature. Then a solution of methanesulfonic anhydride (295.1 mg, 1.69 mmol) in CH₂Cl₂ (1.0 mL) was added dropwise to the mixture at 0° C. The resulting mixture was stirred at room temperature for 16 h. After the reaction was completed, the mixture was diluted with H₂O and extracted with CH₂Cl₂. The combined organic layer was washed with brine, dried over anhydrous sodium sulfate and filtered. The filtrate was concentrated under vacuum to afford tert-butyl (3E)-3-[2-(methanesulfonyloxy)ethylidene]-2-oxopyrrolidine-1-carboxylate (300.0 mg, crude) as dark yellow oil. LCMS (ESI, m/z): [M+H]⁺=306.1.

### Step 2. Synthesis of Tert-butyl (E)-3-(2-(bis(methyl-d₃)amino)ethylidene)-2-oxopyrrolidine-1-carboxylate

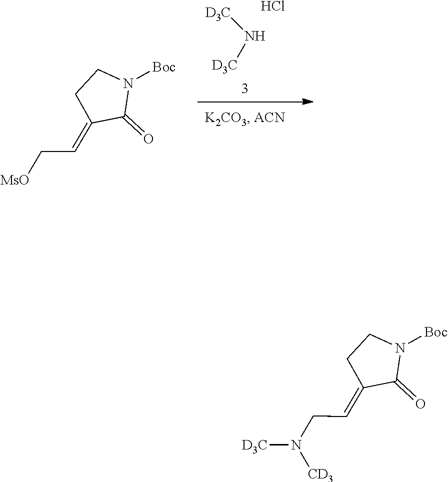

To a solution of dimethyl-d6-amine hydrochloride (172.1 mg, 1.96 mmol) in ACN (12.0 mL) was added K₂CO₃ (678.9 mg, 4.91 mmol) and tert-butyl (E)-3-(2-((methylsulfonyl)oxy)ethylidene)-2-oxopyrrolidine-1-carboxylate (300.0 mg, 0.98 mmol) at room temperature. The resulting mixture was stirred at 60° C. for 2 h. After the reaction was completed, the mixture was diluted with H₂O and extracted with CH₂Cl₂. The combined organic layer was washed with brine, dried over anhydrous sodium sulfate and filtered. The filtrate was concentrated under vacuum. The residue was purified by flash column chromatography with CH₂Cl₂/MeOH (10/1, v/v) to afford tert-butyl (E)-3-(2-(bis(methyl-d₃)amino)ethylidene)-2-oxopyrrolidine-1-carboxylate (100.0 mg, 39%) as a dark yellow oil. LCMS (ESI, m/z): [M+H]⁺=261.2.

### Step 3. Synthesis of (E)-3-(2-(bis(methyl-d3)amino)ethylidene)pyrrolidin-2-one 2,2,2-trifluoroacetate

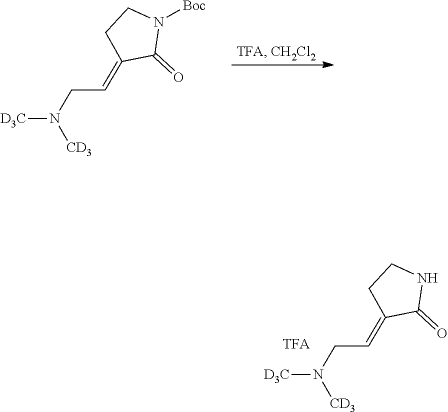

To a solution of tert-butyl (E)-3-(2-(bis(methyl-d3)amino)ethylidene)-2-oxopyrrolidine-1-carboxylate (100.0 mg, 0.38 mmol) in CH₂Cl₂ (2.0 mL) was added TFA (1.0 mL) at room temperature. The mixture was stirred at room temperature for 1 h. After the reaction was completed, the reaction mixture was concentrated under reduced pressure to afford (E)-3-(2-(bis(methyl-d3)amino)ethylidene)pyrrolidin-2-one 2,2,2-trifluoroacetate (250.0 mg, crude) as a dark yellow oil. LCMS (ESI, m/z): [M+H]⁺=161.1.

### Step 4. Synthesis of (E)-3-(2-(bis(methyl-d₃)amino)ethylidene)-1-(4-((3-methyl-4-((1-methyl-1H-benzo[d]imidazol-5-yl)oxy)phenyl)amino)pyrido[3,2-d]pyrimidin-6-yl)pyrrolidin-2-one (Compound 105)

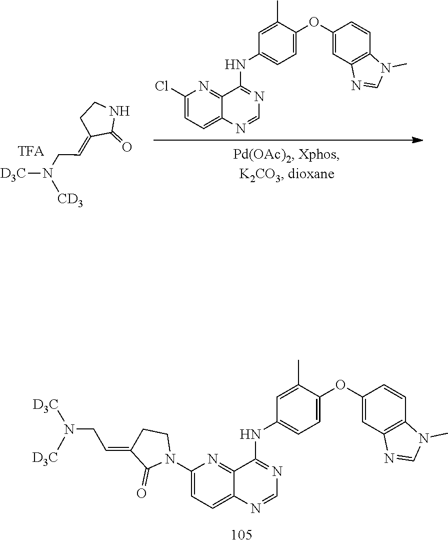 [SMILES: [2H]C([2H])([2H])N(C/C=C1\CCNC1=O)C([2H])([2H])[2H] ; Cc1cc(Nc2ncnc3ccc(Cl)nc23)ccc1Oc1ccc2c(c1)ncn2C ; "Pd(OAc)2, Xphos,K2CO3, dioxane" ; [2H]C([2H])([2H])N(C/C=C1\CCN(c2ccc3ncnc(Nc4ccc(Oc5ccc6c(c5)ncn6C)c(C)c4)c3n2)C1=O)C([2H])([2H])[2H]]

To a mixture of (E)-3-(2-(bis(methyl-d₃)amino)ethylidene)pyrrolidin-2-one 2,2,2-trifluoroacetate (139.4 mg, crude) in dioxane (10.0 mL) was added 6-chloro-N-{3-methyl-4-[(1-methyl-1,3-benzodiazol-5-yl)oxy]phenyl}pyrido[3,2-d]pyrimidin-4-amine (150.0 mg, 0.36 mmol), K₂CO₃ (248.7 mg, 1.80 mmol), Pd(OAc)₂ (8.1 mg, 0.04 mmol) and XPhos (34.3 mg, 0.07 mmol) at room temperature under N₂. The resulting mixture was stirred at 100° C. for 2 h under N₂. After the reaction was completed, the resulting mixture was filtered. The filtrate was concentrated under reduced pressure. The residue was purified by flash column chromatography with CH₂Cl₂/MeOH (10/1, v/v) to afford (E)-3-(2-(bis(methyl-d₃)amino)ethylidene)-1-(4-((3-methyl-4-((1-methyl-1H-benzo[d]imidazol-5-yl)oxy)phenyl)amino)pyrido[3,2-d]pyrimidin-6-yl)pyrrolidin-2-one (Compound 105) (53.5 mg, 27%) as a yellow solid. LCMS (ESI, m/z): [M+H]⁺=541.2. ¹H NMR (400 MHz, DMSO-d₆): δ 9.53 (s, 1H), 8.97 (d, J=9.2 Hz, 1H), 8.59 (s, 1H), 8.25 (d, J=9.2 Hz, 1H), 8.18 (s, 1H), 7.83 (d, J=2.4 Hz, 1H), 7.79-7.76 (m, 1H), 7.58 (d, J=8.8 Hz, 1H), 7.10 (d, J=2.0 Hz, 1H), 7.02-6.99 (m, 1H), 6.91 (d, J=8.8 Hz, 1H), 6.58-6.55 (m, 1H), 4.36-4.32 (m, 2H), 3.85 (s, 3H), 3.16 (d, J=6.8 Hz, 2H), 2.89-2.85 (m, 2H), 2.27 (s, 3H).

### Example S95: Synthesis of (E)-3-(2-(bis(methyl-d₃)amino)ethylidene)-1-(4-((3-methyl-4-((1-(methyl-d₃)-1H-benzo[d]imidazol-5 yl)oxy)phenyl)amino)pyrido[3,2-d]pyrimidin-6-yl)pyrrolidin-2-one (Compound 106)

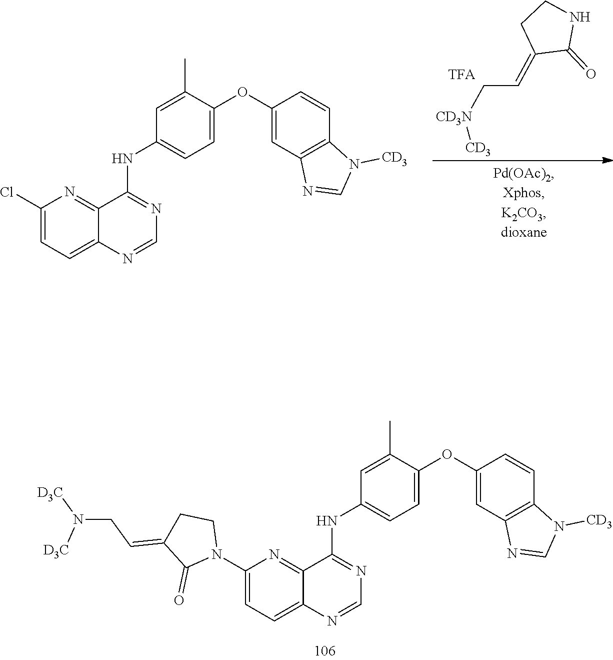

To a mixture of (E)-3-(2-(bis(methyl-d3)amino)ethylidene)pyrrolidin-2-one 2,2,2-trifluoroacetate (350.6 mg, crude) in dioxane (10.0 mL) was added 6-chloro-N-(3-methyl-4-((1-(methyl-d₃)-1H-benzo[d]imidazol-5-yl)oxy)phenyl)pyrido[3,2-d]pyrimidin-4-amine (190.0 mg, 0.45 mmol), K₂CO₃ (312.7 mg, 2.26 mmol), XPhos (43.1 mg, 0.09 mmol) and Pd(OAc)₂ (10.2 mg, 0.05 mmol) at room temperature under N₂. The resulting mixture was stirred at 100° C. for 2 h under N₂. After the reaction was completed, the mixture was filtered. The filtrate was concentrated under reduced pressure. The residue was purified by flash column chromatography with CH₂Cl₂/MeOH (10/1, v/v) and then purified by Prep-HPLC with the following conditions: (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 38% B to 50% B in 8 min; Wave Length: 254 nm) to afford (E)-3-(2-(bis(methyl-d₃)amino)ethylidene)-1-(4-((3-methyl-4-((1-(methyl-d₃)-1H-benzo[d]imidazol-5-yl)oxy)phenyl)amino)pyrido[3,2-d]pyrimidin-6-yl)pyrrolidin-2-one (Compound 106) (43.0 mg, 17%) as a yellow solid. LCMS (ESI, m/z): [M+H]⁺=544.2. ¹H NMR (400 MHz, DMSO-d₆): δ 9.52 (s, 1H), 8.98-8.96 (m, 1H), 8.59 (s, 1H), 8.26-8.23 (m, 1H), 8.17 (s, 1H), 7.83 (s, 1H), 7.79-7.76 (m, 1H), 7.59-7.56 (m, 1H), 7.11 (s, 1H), 7.02-6.99 (m, 1H), 6.92-6.90 (m, 1H), 6.59-6.54 (m, 1H), 4.36-4.32 (m, 2H), 3.12-3.09 (m, 2H), 2.88-2.84 (m, 2H), 2.27 (s, 3H).

### Example S96: Synthesis of (E)-3-(2-(dimethylamino)ethylidene)-1-(4-((3-methyl-4-((1-(methyl-d3)-1H-benzo[d]imidazol-5 yl)oxy)phenyl)amino)pyrido[3,2-d]pyrimidin-6-yl)pyrrolidin-2-one (Compound 119)

### Step 1. Synthesis of 4-bromo-N-(methyl-d3)-2-nitroaniline

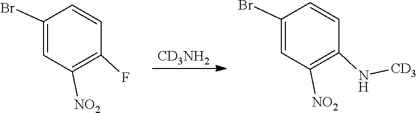

To a solution of 4-bromo-1-fluoro-2-nitrobenzene (10.0 g, 45.46 mmol) in ACN (100.0 mL)/THF (100.0 mL) was added TEA (27.6 g, 272.73 mmol) and CD₃NH₂ (7.7 g, 227.14 mmol) at room temperature. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to 4-bromo-N-(methyl-d3)-2-nitroaniline (10.0 g, 93%) as a yellow solid. LCMS (ESI, m/z): [M+H]⁺=234.0.

### Step 2. Synthesis of 4-bromo-N1-(methyl-d3)benzene-1,2-diamine

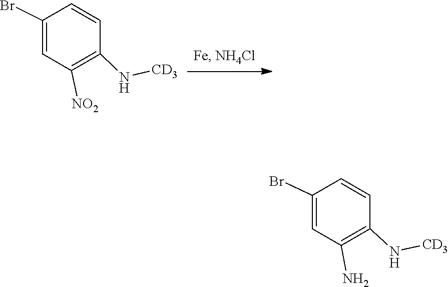 [SMILES: [2H]C([2H])([2H])Nc1ccc(Br)cc1[N+](=O)[O-] ; [2H]C([2H])([2H])Nc1ccc(Br)cc1N]

To a solution of 4-bromo-N-(methyl-d3)-2-nitroaniline (10.0 g, 42.72 mmol) in EtOH (100.0 mL)/H₂O (20.0 mL) was added NH₄Cl (6.8 g, 128.16 mmol) and Fe (7.6 g, 128.16 mmol) at room temperature. The resulting mixture was stirred at 80° C. for 3 h. After the reaction was completed, the resulting mixture was filtered. The filtrate was concentrated under reduced pressure. The residue was purified by flash column chromatography with CH₂Cl₂/MeOH (10/1, v/v) to afford 4-bromo-N1-(methyl-d3)benzene-1,2-diamine (8.0 g, 91%) as a black oil. LCMS (ESI, m/z): [M+H]⁺=204.0.

### Step 3. Synthesis of 5-bromo-1-(methyl-d3)-1H-benzo[d]imidazole

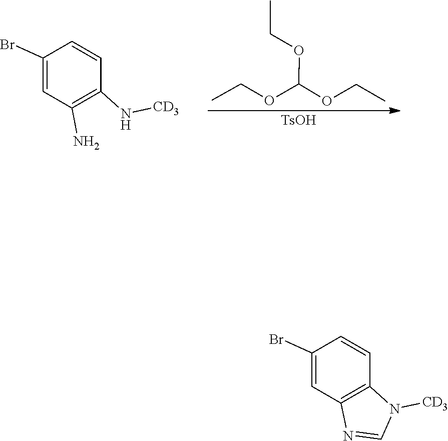

To a solution of 4-bromo-N1-(methyl-d3)benzene-1,2-diamine (8.0 g, 39.19 mmol) in triethyl orthoformate (80.0 mL) was added TsOH (10.1 g, 58.79 mmol) at room temperature. The resulting mixture was stirred at 80° C. for 2 h. After the reaction was completed, the resulting mixture was concentrated under reduced pressure. The residue was purified by flash column chromatography with CH₂Cl₂/MeOH (10/1, v/v) to afford 5-bromo-1-(methyl-d3)-1H-benzo[d]imidazole (10.0 g, crude) as a white solid. LCMS (ESI, m/z): [M+H]⁺=214.0.

### Step 4. Synthesis of 1-(methyl-d3)-1H-benzo[d]imidazol-5-ol

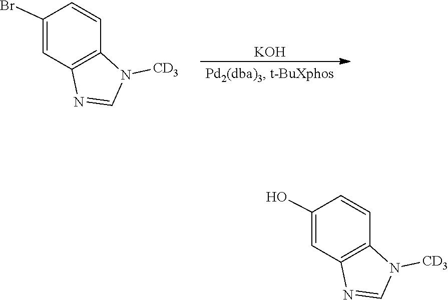 [SMILES: [2H]C([2H])([2H])n1cnc2cc(Br)ccc21 ; "KOH" ; [2H]C([2H])([2H])n1cnc2cc(O)ccc21]

To a mixture of 5-bromo-1-(methyl-d3)-1H-benzo[d]imidazole (7.0 g, 32.69 mmol) in dioxane (70.0 mL)/H₂O (14.0 mL) was added KOH (3.6 g, 65.39 mmol), t-BuXPhos (2.7 g, 6.54 mmol) and Pd₂(dba)₃ (2.9 g, 3.27 mmol) at room temperature under N₂. The resulting mixture was stirred at 80° C. for 4 h. After the reaction was completed, the resulting mixture was concentrated under reduced pressure. The residue was purified by flash column chromatography with CH₂Cl₂/MeOH (10/1, v/v) to afford 1-(methyl-d3)-1H-benzo[d]imidazol-5-ol (750.0 mg, 15%) as a light brown solid. LCMS (ESI, m/z): [M+H]⁺=152.1.

### Step 5. Synthesis of 1-(methyl-d3)-5-(2-methyl-4-nitrophenoxy)-1H-benzo[d]imidazole

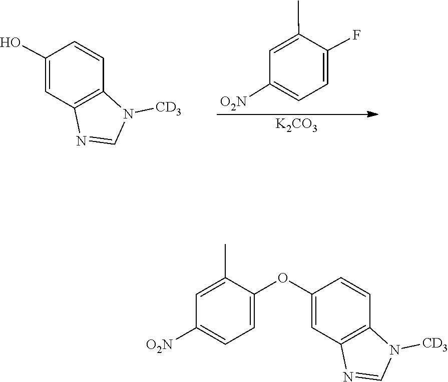

To a solution of 1-(methyl-d3)-1H-benzo[d]imidazol-5-ol (750.0 mg, 4.96 mmol) in DMF (10.0 mL) was added 1-fluoro-2-methyl-4-nitrobenzene (1381.2 mg, 9.92 mmol) and K₂CO₃ (1399.9 mg, 9.92 mmol) at room temperature. The resulting mixture was stirred at 50° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 1-(methyl-d3)-5-(2-methyl-4-nitrophenoxy)-1H-benzo[d]imidazole (400.0 mg, 29%) as a white solid. LCMS (ESI, m/z): [M+H]⁺=287.1

### Step 6. Synthesis of 3-methyl-4-((1-(methyl-d3)-1H-benzo[d]imidazol-5-yl)oxy)aniline

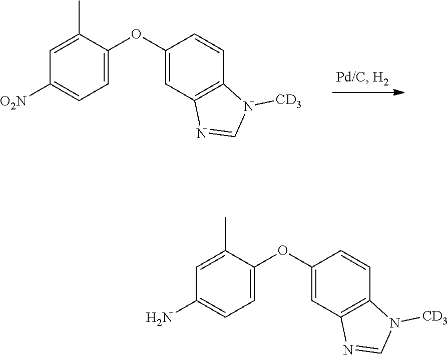 [SMILES: [2H]C([2H])([2H])n1cnc2cc(Oc3ccc([N+](=O)[O-])cc3C)ccc21 ; [2H]C([2H])([2H])n1cnc2cc(Oc3ccc(N)cc3C)ccc21]

To a solution of 1-(methyl-d3)-5-(2-methyl-4-nitrophenoxy)-1H-benzo[d]imidazole (750.0 mg, 2.62 mmol) in MeOH (100.0 mL) was added Pd/C (225.8 mg, dry) at room temperature under N₂. The mixture was stirred at room temperature for 4 h under H₂. After the reaction was completed, the resulting mixture was filtered. The filtrate was concentrated under reduced pressure. The residue was purified by flash column chromatography with CH₂Cl₂/MeOH (10/1 v/v) to afford 3-methyl-4-((1-(methyl-d3)-1H-benzo[d]imidazol-5-yl)oxy)aniline (500.0 mg, 74%) as a white solid. LCMS (ESI, m/z): [M+H]⁺=257.1.

### Step 7. Synthesis of 6-chloro-N-(3-methyl-4-((1-(methyl-d3)-1H-benzo[d]imidazol-5-yl)oxy)phenyl)pyrido[3,2-d]pyrimidin-4-amine

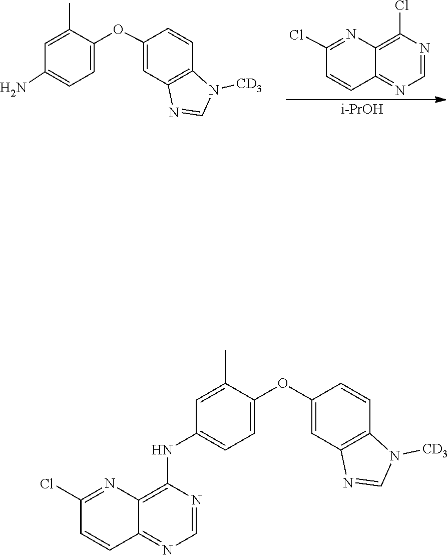 [SMILES: "i-PrOH" ; [2H]C([2H])([2H])n1cnc2cc(Oc3ccc(N)cc3C)ccc21 ; Clc1ccc2ncnc(Cl)c2n1 ; [2H]C([2H])([2H])n1cnc2cc(Oc3ccc(Nc4ncnc5ccc(Cl)nc45)cc3C)ccc21]

To a solution of 4,6-dichloropyrido[3,2-d]pyrimidine (702.3 mg, crude) in i-PrOH (20.0 mL) was added 3-methyl-4-((1-(methyl-d3)-1H-benzo[d]imidazol-5-yl)oxy)aniline (750.0 mg, 2.90 mmol) at 0° C. The mixture was stirred at room temperature for 2 h. After the reaction was completed, the resulting mixture was concentrated under reduced pressure. The residue was purified by flash column chromatography with CH₂Cl₂/MeOH (10/1 v/v) to afford 6-chloro-N-(3-methyl-4-((1-(methyl-d3)-1H-benzo[d]imidazol-5-yl)oxy)phenyl)pyrido[3,2-d]pyrimidin-4-amine (1.1 g, 95%) as a yellow solid. LCMS (ESI, m/z): [M+H]⁺=420.1.

### Step 8. Synthesis of (E)-3-(2-(dimethylamino)ethylidene)-1-(4-((3-methyl-4-((1-(methyl-d3)-1H-benzo[d]imidazol-5-yl)oxy)phenyl)amino)pyrido[3,2-d]pyrimidin-6-yl)pyrrolidin-2-one (Compound 119)

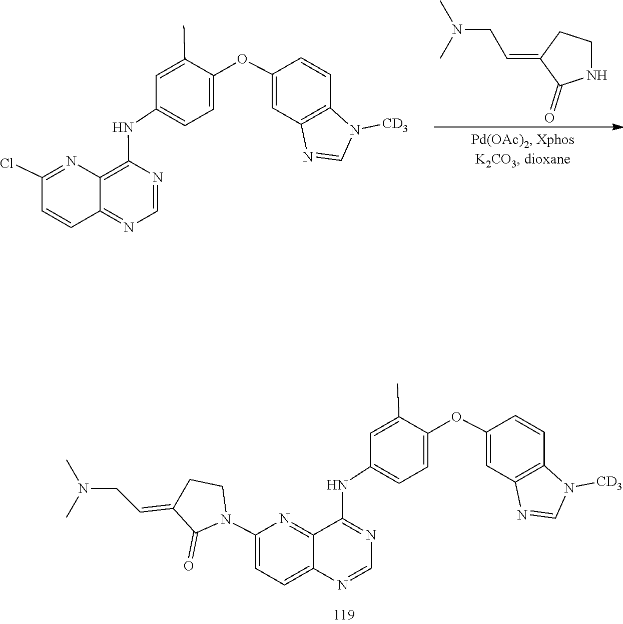 [SMILES: [2H]C([2H])([2H])n1cnc2cc(Oc3ccc(Nc4ncnc5ccc(Cl)nc45)cc3C)ccc21 ; CN(C)C/C=C1\CCNC1=O ; [2H]C([2H])([2H])n1cnc2cc(Oc3ccc(Nc4ncnc5ccc(N6CC/C(=C\CN(C)C)C6=O)nc45)cc3C)ccc21]

To a solution of 6-chloro-N-(3-methyl-4-((1-(methyl-d3)-1H-benzo[d]imidazol-5-yl)oxy)phenyl)pyrido[3,2-d]pyrimidin-4-amine (175.0 mg, 0.41 mmol) in dioxane (10.0 mL) was added (3E)-3-[2-(dimethylamino)ethylidene]pyrrolidin-2-one (64.2 mg, 0.41 mmol), K₂CO₃ (115.2 mg, 0.83 mmol), XPhos (39.7 mg, 0.08 mmol) and Pd(OAc)₂ (9.3 mg, 0.04 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: (Column: Xselect CSH Column 30×150 mm, 5 μm; Mobile Phase A: Water (0.1% FA), Mobile Phase B: ACN; Flow rate: 60 mL/min; Gradient: 8% B to 22% B in 10 min; Wave Length: 220 nm) to afford (E)-3-(2-(dimethylamino)ethylidene)-1-(4-((3-methyl-4-((1-(methyl-d3)-1H-benzo[d]imidazol-5-yl)oxy)phenyl)amino)pyrido[3,2-d]pyrimidin-6-yl)pyrrolidin-2-one (Compound 119) (15.1 mg, 6%) as a white solid. LCMS (ESI, m/z): [M+H]⁺=538.2. ¹H NMR (400 MHz, DMSO-d₆): δ 9.50 (s, 1H), 8.96 (d, J=9.2 Hz, 1H), 8.58 (s, 1H), 8.27-8.22 (m, 1H), 8.17 (s, 1H), 7.83-7.76 (m, 2H), 7.57 (d, J=8.4 Hz, 1H), 7.10 (d, J=2.0 Hz, 1H), 7.01-6.99 (m, 1H), 6.90 (d, J=8.8 Hz, 1H), 6.58-6.54 (m, 1H), 4.34-4.31 (m, 2H), 3.10 (d, J=6.4 Hz, 2H), 2.87-2.83 (m, 2H), 2.27 (s, 3H), 2.20 (s, 6H).

### Example S97: Synthesis of (3E,4R)-3 [2-(dimethylamino)ethylidene]-4-methyl-1-[4-({3-methyl-4-[(1-methyl-1,3-benzodiazol-5 yl)oxy]phenyl}amino)pyrido[3,4-d]pyrimidin-6-yl]pyrrolidin-2-one and (3E,4S)-3-[2-(dimethylamino)ethylidene]-4-methyl-1-[4-([{3-methyl-4-[(1-methyl-1,3-benzodiazol-5-yl)oxy]phenyl}amino)pyrido[3,4-d]pyrimidin-6-yl]pyrrolidin-2-one (Compound 120 and Compound 121)

### Step 1. Synthesis of (3E)-3-[2-(dimethylamino)ethylidene]-4-methyl-1-[4-({3-methyl-4-[(1-methyl-1,3-benzodiazol-5-yl)oxy]phenyl}amino)pyrido[3,4-d]pyrimidin-6-yl]pyrrolidin-2-one

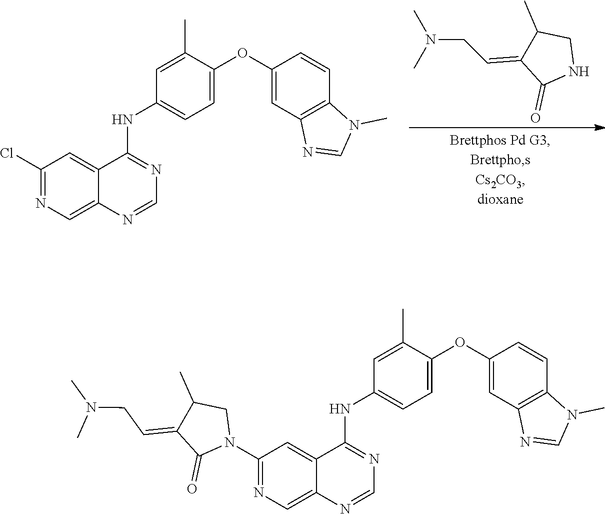

To a solution of 6-chloro-N-{3-methyl-4-[(1-methyl-1,3-benzodiazol-5-yl)oxy]phenyl}pyrido[3,4-d]pyrimidin-4-amine (150.0 mg, 0.36 mmol) in dioxane (5.0 mL) was added (3E)-3-[2-(dimethylamino)ethylidene]-4-methylpyrrolidin-2-one (60.5 mg, 0.36 mmol), Cs₂CO₃ (351.7 mg, 1.08 mmol), BrettPhos (38.6 mg, 0.07 mmol) and BrettPhos Pd G3 (32.6 mg, 0.03 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash chromatography with CH₂Cl₂/MeOH (5/1, v/v) and then purified by Prep-HPLC with the following conditions: (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 34% B to 44% B in 8 min; Wave Length: 254 nm) to afford (3E)-3-[2-(dimethylamino)ethylidene]-4-methyl-1-[4-({3-methyl-4-[(1-methyl-1,3-benzodiazol-5-yl)oxy]phenyl}amino)pyrido[3,4-d]pyrimidin-6-yl]pyrrolidin-2-one (75.0 mg, 37%) as a yellow solid. LCMS (ESI, m/z): [M+H]⁺=549.3.

### Step 2. Synthesis of (3E,4R)-3-[2-(dimethylamino)ethylidene]-4-methyl-1-[4-({3-methyl-4-[(1-methyl-1,3-benzodiazol-5-yl)oxy]phenyl}amino)pyrido[3,4-d]pyrimidin-6-yl]pyrrolidin-2-one and (3E,4S)-3-[2-(dimethylamino)ethylidene]-4-methyl-1-[4-({3-methyl-4-[(1-methyl-1,3-benzodiazol-5-yl)oxy]phenyl}amino)pyrido[3,4-d]pyrimidin-6-yl]pyrrolidin-2-one (Compound 120 and Compound 121)

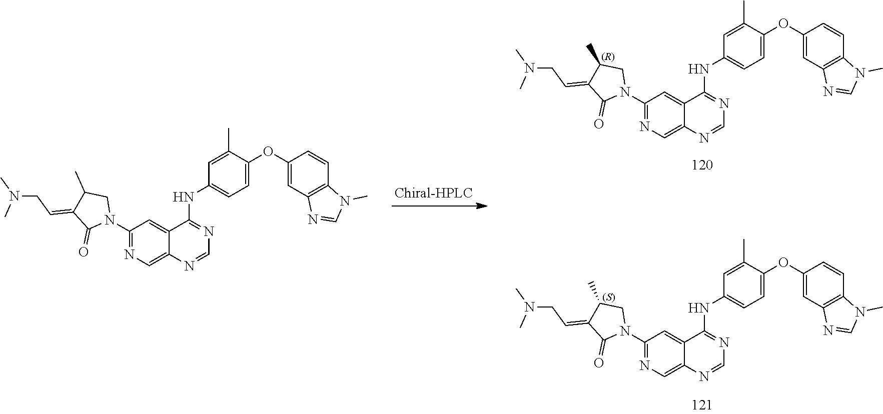

The racemic mixture of (3E)-3-[2-(dimethylamino)ethylidene]-4-methyl-1-[4-({3-methyl-4-[(1-methyl-1,3-benzodiazol-5-yl)oxy]phenyl}amino)pyrido[3,4-d]pyrimidin-6-yl]pyrrolidin-2-one (75.0 mg, 0.13 mmol) was separated by Prep-Chiral-HPLC with the following conditions (Column: CHIRAL ART Cellulose-SB, 2×25 cm, 5 μm; Mobile Phase A: Hex (0.5% 2M NH₃-MeOH)-HPLC, Mobile Phase B: EtOH: DCM=1: 1-HPLC; Flow rate: 20 mL/min; Gradient: 40% B to 40% B in 10 min; Wave Length: 220/254 nm; RT1(min): 6.91; RT2(min): 8.19) to afford 3-[2-(dimethylamino)ethylidene]-4-methyl-1-[4-({3-methyl-4-[(1-methyl-1,3-benzodiazol-5-yl)oxy]phenyl}amino)pyrido[3,4-d]pyrimidin-6-yl]pyrrolidin-2-one Enantiomer 1 (RT1(min): 6.91, 20.3 mg, 54%) as a yellow solid and 3-[2-(dimethylamino)ethylidene]-4-methyl-1-[4-({3-methyl-4-[(1-methyl-1,3-benzodiazol-5-yl)oxy]phenyl}amino)pyrido[3,4-d]pyrimidin-6-yl]pyrrolidin-2-one Enantiomer 2 (RT2(min): 8.19, 15.7 mg, 41%) as a yellow solid. The absolute stereochemistry of Enantiomers 1 and 2 was not assigned. The two enantiomeric structures that could be obtained from chiral separation of the enantiomeric mixture as described above are shown as Compounds 120 and 121 in Table 1.

3-[2-(dimethylamino)ethylidene]-4-methyl-1-[4-({3-methyl-4-[(1-methyl-1,3-benzodiazol-5-yl)oxy]phenyl}amino)pyrido[3,4-d]pyrimidin-6-yl]pyrrolidin-2-one Enantiomer 1: RT1(min): 6.91; LCMS (ESI, m/z): [M+H]⁺=549.4. ¹H NMR (400 MHz, DMSO-d₆): δ 10.24 (s, 1H), 9.17 (s, 1H), 9.05 (s, 1H), 8.59 (s, 1H), 8.18 (s, 1H), 7.74 (s, 1H), 7.63-7.57 (m, 2H), 7.13 (d, J=2.0 Hz, 1H), 7.03-7.00 (m, 1H), 6.86 (d, J=8.8 Hz, 1H), 6.54-6.50 (m, 1H), 4.27-4.22 (m, 1H), 3.84-3.78 (m, 4H), 3.29-3.20 (m, 2H), 3.14-3.08 (m, 1H), 2.26-2.20 (m, 9H), 1.24 ((d, J=6.8 Hz, 3H).

3-[2-(dimethylamino)ethylidene]-4-methyl-1-[4-({3-methyl-4-[(1-methyl-1,3-benzodiazol-5-yl)oxy]phenyl}amino)pyrido[3,4-d]pyrimidin-6-yl]pyrrolidin-2-one Enantiomer 2: RT2(min): 8.19; LCMS (ESI, m/z): [M+H]⁺=549.4. ¹H NMR (400 MHz, DMSO-d₆): δ 10.24 (s, 1H), 9.17 (s, 1H), 9.05 (s, 1H), 8.59 (s, 1H), 8.18 (s, 1H), 7.74 (s, 1H), 7.63-7.57 (m, 2H), 7.13 (d, J=2.4 Hz, 1H), 7.03-7.00 (m, 1H), 6.86 (d, J=8.8 Hz, 1H), 6.54-6.50 (m, 1H), 4.26-4.22 (m, 1H), 3.84-3.78 (m, 4H), 3.29-3.20 (m, 2H), 3.13-3.08 (m, 1H), 2.26-2.20 (m, 9H), 1.24 ((d, J=6.8 Hz, 3H).

### Example S98: Synthesis of (3E,4R)-3 [2-(dimethylamino)ethylidene]-4-methyl-1-{4-[(3-methyl-4-{[1,2,4]triazolo[1,5-a]pyridin-7-yloxy}phenyl)amino]pyrido[3,2-d]pyrimidin-6-yl}pyrrolidin-2-one and (3E,4S)-3-[2-(dimethylamino)ethylidene]-4-methyl-1-{4-[(3-methyl-4-{[1,2,4]triazolo[1,5-a]pyridin-7-yloxy}phenyl)amino]pyrido[3,2-d]pyrimidin-6-yl}pyrrolidin-2-one (Compound 122 and Compound 123)

### Step 1. Synthesis of (3E)-3-[2-(dimethylamino)ethylidene]-4-methyl-1-{4-[(3-methyl-4-{[1,2,4]triazolo[1,5-a]pyridin-7-yloxy}phenyl)amino]pyrido[3,2-d]pyrimidin-6-yl}pyrrolidin-2-one

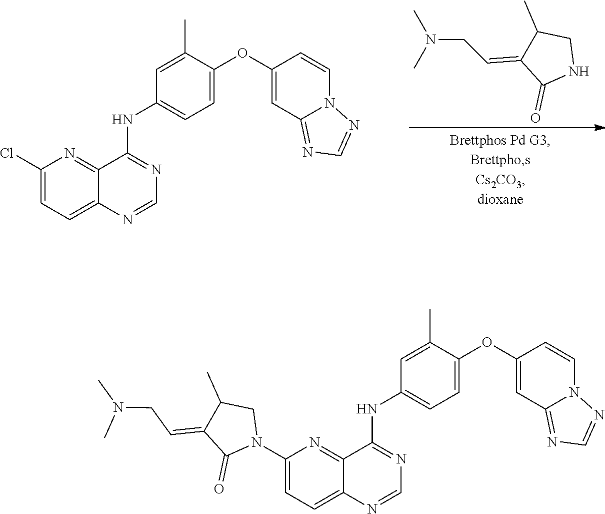 [SMILES: Cc1cc(Nc2ncnc3ccc(Cl)nc23)ccc1Oc1ccn2ncnc2c1 ; CC1CNC(=O)/C1=C/CN(C)C ; Cc1cc(Nc2ncnc3ccc(N4CC(C)/C(=C\CN(C)C)C4=O)nc23)ccc1Oc1ccn2ncnc2c1]

To a solution of 6-chloro-N-(3-methyl-4-{[1,2,4]triazolo[1,5-a]pyridin-7-yloxy}phenyl)pyrido[3,2-d]pyrimidin-4-amine (200.0 mg, 0.50 mmol) in 1,4-dioxane (5.0 mL) were added (3E)-3-[2-(dimethylamino)ethylidene]-4-methylpyrrolidin-2-one (83.3 mg, 0.50 mmol), Cs₂CO₃ (322.7 mg, 1.00 mmol), BrettPhos Pd G3 (44.9 mg, 0.05 mmol) and BrettPhos (53.2 mg, 0.10 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford (3E)-3-[2-(dimethylamino)ethylidene]-4-methyl-1-{4-[(3-methyl-4-{[1,2,4]triazolo[1,5-a]pyridin-7-yloxy}phenyl)amino]pyrido[3,2-d]pyrimidin-6-yl}pyrrolidin-2-one (50.0 mg, 18%) as a yellow solid. LCMS (ESI, m/z): [M+H]⁺=536.2.

### Step 2. Synthesis of (3E,4R)-3-[2-(dimethylamino)ethylidene]-4-methyl-1-{4-[(3-methyl-4-{[1,2,4]triazolo[1,5-a]pyridin-7-yloxy}phenyl)amino]pyrido[3,2-d]pyrimidin-6-yl}pyrrolidin-2-one and (3E,4S)-3-[2-(dimethylamino)ethylidene]-4-methyl-1-{4-[(3-methyl-4-{[1,2,4]triazolo[1,5-a]pyridin-7-yloxy}phenyl)amino]pyrido[3,2-d]pyrimidin-6-yl}pyrrolidin-2-one (Compound 122 and Compound 123)

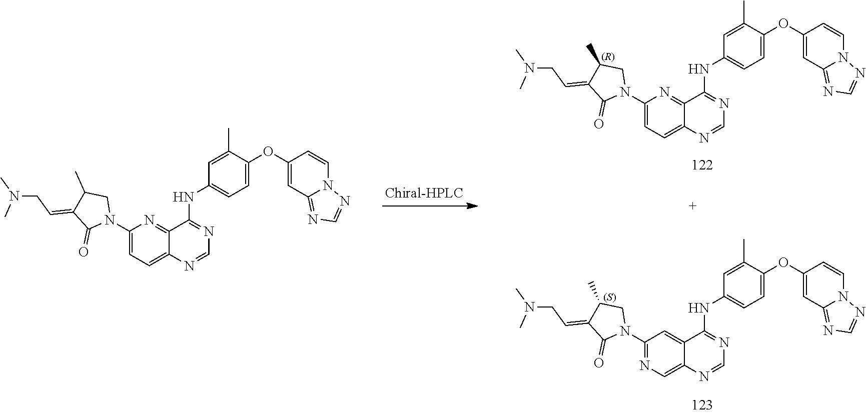

The product of (3E)-3-[2-(dimethylamino)ethylidene]-4-methyl-1-{4-[(3-methyl-4-{[1,2,4]triazolo[1,5-a]pyridin-7-yloxy}phenyl)amino]pyrido[3,2-d]pyrimidin-6-yl}pyrrolidin-2-one (50.0 mg, 0.09 mmol) was separated by Prep-Chiral-HPLC with the following conditions: (Column: CHIRAL ART Cellulose-SB, 2×25 cm, 5 μm; Mobile Phase A: Hex (0.5% 2M NH₃-MeOH)-HPLC, Mobile Phase B: MeOH: DCM=1: 1-HPLC; Flow rate: 20 mL/min; Gradient: 30% B to 30% B in 9 min; Wave Length: 220/254 nm; RT1(min): 6.18; RT2(min): 7.33) to afford 3-[2-(dimethylamino)ethylidene]-4-methyl-1-{4-[(3-methyl-4-{[1,2,4]triazolo[1,5-a]pyridin-7-yloxy}phenyl)amino]pyrido[3,2-d]pyrimidin-6-yl}pyrrolidin-2-one Enantiomer 1 (RT1(min): 6.18, 14.4 mg, 57%) as a yellow solid and 3-[2-(dimethylamino)ethylidene]-4-methyl-1-{4-[(3-methyl-4-{[1,2,4]triazolo[1,5-a]pyridin-7-yloxy}phenyl)amino]pyrido[3,2-d]pyrimidin-6-yl}pyrrolidin-2-one Enantiomer 2 (RT2(min): 7.33, 14.2 mg, 56%) as a yellow solid. The absolute stereochemistry of Enantiomers 1 and 2 was not assigned. The two enantiomeric structures that could be obtained from chiral separation of the enantiomeric mixture as described above are shown as Compounds 122 and 123 in Table 1.

3-[2-(dimethylamino)ethylidene]-4-methyl-1-{4-[(3-methyl-4-{[1,2,4]triazolo[1,5-a]pyridin-7-yloxy}phenyl)amino]pyrido[3,2-d]pyrimidin-6-yl}pyrrolidin-2-one Enantiomer 1: RT1(min): 6.18; LCMS (ESI, m/z): [M+H]⁺=536.3. ¹H NMR (400 MHz, DMSO-d₆): δ 9.59 (s, 1H), 9.00-8.94 (m, 2H), 8.63 (s, 1H), 8.39 (s, 1H), 8.26 (d, J=9.2 Hz, 1H), 7.97-7.95 (m, 2H), 7.26 (d, J=8.4 Hz, 1H), 7.05-7.03 (m, 1H), 6.80 (d, J=1.6 Hz, 1H), 6.58-6.55 (m, 1H), 4.34-4.29 (m, 1H), 4.15-4.12 (m, 1H), 3.29-3.12 (m, 3H), 2.22 (s, 9H), 1.27 (d, J=7.2 Hz, 3H).

3-[2-(dimethylamino)ethylidene]-4-methyl-1-{4-[(3-methyl-4-{[1,2,4]triazolo[1,5-a]pyridin-7-yloxy}phenyl)amino]pyrido[3,2-d]pyrimidin-6-yl}pyrrolidin-2-one Enantiomer 2: RT2(min): 7.33; LCMS (ESI, m/z): [M+H]⁺=536.1. ¹H NMR (400 MHz, DMSO-d₆): δ 9.60 (s, 1H), 9.01-8.94 (m, 2H), 8.63 (s, 1H), 8.39 (s, 1H), 8.27 (d, J=9.2 Hz, 1H), 7.98-7.94 (m, 2H), 7.26 (d, J=8.4 Hz, 1H), 7.04 (d, J=5.6 Hz, 1H), 6.80 (s, 1H), 6.57-6.55 (m, 1H), 4.35-4.30 (m, 1H), 4.15-4.12 (m, 1H), 3.27-3.11 (m, 3H), 2.22 (s, 9H), 1.27 (d, J=6.8 Hz, 3H).

### Example S99: Synthesis of (3E)-3 [2-(dimethylamino)ethylidene]-1-{4-[(3-methyl-4-{[1,2,3,4]tetrazolo[1,5-a]pyridin-7-yloxy}phenyl)amino]pyrido[3,4-d]pyrimidin-6-yl}pyrrolidin-2-one (Compound 107)

### Step 1. Synthesis of 7-(benzyloxy)-[1,2,3,4]tetrazolo[1,5-a]pyridine

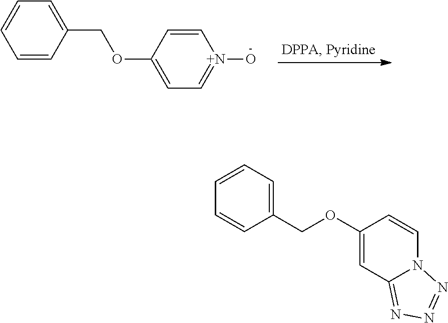 [SMILES: O[n+]1ccc(OCc2ccccc2)cc1 ; c1ccc(COc2ccn3nnnc3c2)cc1]

To a solution of 4-(benzyloxy)pyridin-1-ium-1-olate (6.0 g, 29.82 mmol) in Pyridine (4.8 mL) was added DPPA (16.4 g, 59.63 mmol) at room temperature. The resulting mixture was stirred at 100° C. for 24 h. After the reaction was completed, the resulting mixture was diluted with water and extracted with ethyl acetate. The combined organic layers were washed with brine, dried over anhydrous sodium sulfate and filtered. The filtrate was concentrated under reduced pressure. The residue The residue was purified by flash column chromatography with CH₂Cl₂/MeOH (10/1, v/v) to afford 7-(benzyloxy)-[1,2,3,4]tetrazolo[1,5-a]pyridine (3.0 g, 44%) as a white solid. LCMS (ESI, m/z): [M+H]⁺=227.1.

Step 2. Synthesis of [1,2,3,4]tetrazolo[1,5-a]pyridin-7-ol

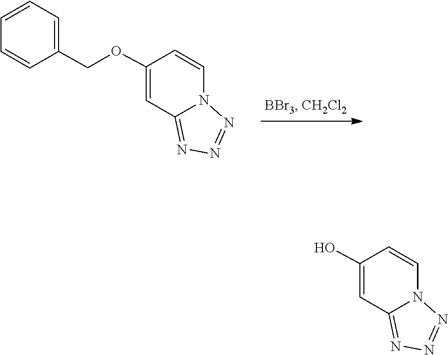 [SMILES: c1ccc(COc2ccn3nnnc3c2)cc1 ; Oc1ccn2nnnc2c1]

To a solution of 7-(benzyloxy)-[1,2,3,4]tetrazolo[1,5-a]pyridine (2.9 g, 12.82 mmol) in DCM (20.0 mL) was added a solution boron tribromide in CH₂Cl₂ (25.6 mL, 1 mol/L) at room temperature. The resulting mixture was stirred at room temperature for 4 h. After the reaction was completed, the reaction mixture was quenched with EtOH at 0° C. the resulting mixture was concentrated under reduced pressure. The residue was purified by flash column chromatography with CH₂Cl₂/MeOH (9/1, v/v) to afford [1,2,3,4]tetrazolo[1,5-a]pyridin-7-ol (1.6 g, 45%) as a brown solid. LCMS (ESI, m/z): [M+H]⁺=137.0.

### Step 3. Synthesis of 7-(2-methyl-4-nitrophenoxy)-[1,2,3,4]tetrazolo[1,5-a]pyridine

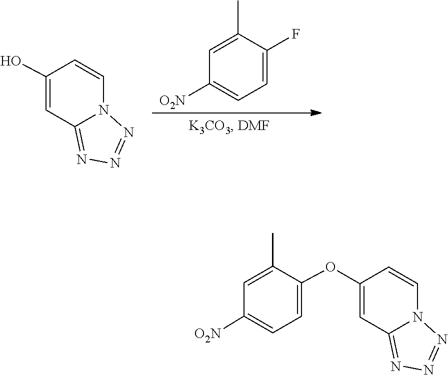 [SMILES: Oc1ccn2nnnc2c1 ; Cc1cc([N+](=O)[O-])ccc1F ; Cc1cc([N+](=O)[O-])ccc1Oc1ccn2nnnc2c1]

To a solution of [1,2,3,4]tetrazolo[1,5-a]pyridin-7-ol (1.58 g, 11.61 mmol) in DMF (15.0 mL) was added 1-fluoro-2-methyl-4-nitrobenzene (1.8 g, 11.61 mmol) and K₂CO₃ (3.2 g, 23.22 mmol) at room temperature. The resulting mixture was stirred at 80° C. for 16 h. After the reaction was completed, the resulting mixture was diluted with water and extracted with ethyl acetate. The combined organic layers were washed with brine, dried over anhydrous sodium sulfate and filtered. The filtrate was concentrated under reduced pressure. The residue was purified by flash column chromatography with CH₂Cl₂/MeOH (5/1, v/v) to afford 7-(2-methyl-4-nitrophenoxy)-[1,2,3,4]tetrazolo[1,5-a]pyridine (800.0 mg, 25%) as a yellow solid. LCMS (ESI, m/z): [M+H]⁺=272.1.

### Step 4. Synthesis of 3-methyl-4-{[1,2,3,4]tetrazolo[1,5-a]pyridin-7-yloxy}aniline

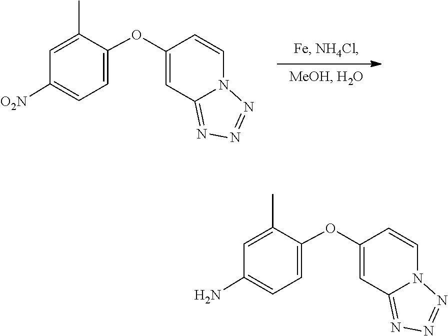 [SMILES: Cc1cc([N+](=O)[O-])ccc1Oc1ccn2nnnc2c1 ; Cc1cc(N)ccc1Oc1ccn2nnnc2c1]

To a solution of 7-(2-methyl-4-nitrophenoxy)-[1,2,3,4]tetrazolo[1,5-a]pyridine (800.0 mg, 2.95 mmol) in MeOH/H₂O (10.0 mL/2.0 mL) was added Fe (230.6 mg, 4.14 mmol) and NH₄Cl (788.8 mg, 14.75 mmol) at room temperature. The resulting mixture was stirred at 80° C. for 2 h. After the reaction was completed, the reaction mixture was filtered. The filtrate was concentrated under reduced pressure. The residue was purified by flash column chromatography with CH₂Cl₂/MeOH (10/1, v/v) to afford 3-methyl-4-{[1,2,3,4]tetrazolo[1,5-a]pyridin-7-yloxy}aniline (300.0 mg, 42%) as a yellow solid. LCMS (ESI, m/z): [M+H]⁺=242.1.

### Step 5. Synthesis of 6-chloro-N-(3-methyl-4-{[1,2,3,4]tetrazolo[1,5-a]pyridin-7-yloxy}phenyl)pyrido[3,2-d]pyrimidin-4-amine

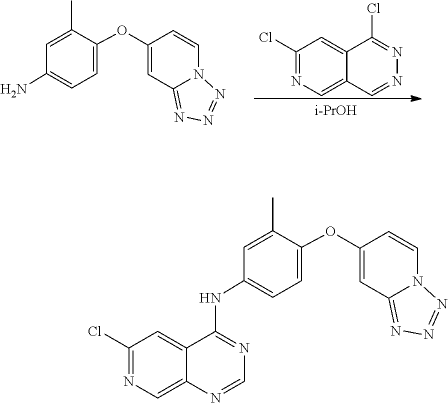 [SMILES: "i-PrOH" ; Cc1cc(N)ccc1Oc1ccn2nnnc2c1 ; Clc1cc2c(Cl)nncc2cn1 ; Cc1cc(Nc2ncnc3cnc(Cl)cc23)ccc1Oc1ccn2nnnc2c1]

To a solution of 3-methyl-4-{[1,2,3,4]tetrazolo[1,5-a]pyridin-7-yloxy}aniline (200.0 mg, 0.83 mmol) in i-PrOH (5.0 mL) was added 4,6-dichloropyrido[3,2-d]pyrimidine (165.8 mg, crude) at room temperature. The resulting mixture was stirred at room temperature for 2 h. After the reaction was completed, the resulting mixture was concentrated under reduced pressure. The residue was purified by flash column chromatography with CH₂Cl₂/MeOH (8/1, v/v) to afford 6-chloro-N-(3-methyl-4-{[1,2,3,4]tetrazolo[1,5-a]pyridin-7-yloxy}phenyl)pyrido[3,2-d]pyrimidin-4-amine (200.0 mg, 59%) as a yellow solid. LCMS (ESI, m/z): [M+H]⁺=405.1.

### Step 6. Synthesis of (3E)-3-[2-(dimethylamino)ethylidene]-1-{4-[(3-methyl-4-{[1,2,3,4]tetrazolo[1,5-a]pyridin-7-yloxy}phenyl)amino]pyrido[3,4-d]pyrimidin-6-yl}pyrrolidin-2-one (Compound 107)

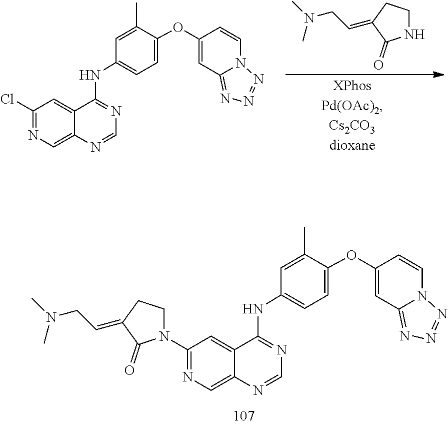 [SMILES: Cc1cc(Nc2ncnc3cnc(Cl)cc23)ccc1Oc1ccn2nnnc2c1 ; CN(C)C/C=C1\CCNC1=O ; Cc1cc(Nc2ncnc3cnc(N4CC/C(=C\CN(C)C)C4=O)cc23)ccc1Oc1ccn2nnnc2c1]

To a solution of 6-chloro-N-(3-methyl-4-{[1,2,3,4]tetrazolo[1,5-a]pyridin-7-yloxy}phenyl)pyrido[3,4-d]pyrimidin-4-amine (140.0 mg, 0.35 mmol) in dioxane (4.0 mL) was added (3E)-3-[2-(dimethylamino)ethylidene]pyrrolidin-2-one (64.0 mg, 0.42 mmol), Cs₂CO₃ (325.8 mg, 1.04 mmol), XPhos (33.0 mg, 0.07 mmol) and Pd(OAc)₂ (7.8 mg, 0.04 mmol) at room temperature under N₂. The resulting mixture was stirred at 100° C. for 2 h. After the reaction was completed, the resulting mixture was diluted with water and extracted with ethyl acetate. The combined organic layers were washed with brine, dried over anhydrous sodium sulfate and filtered. The filtrate was concentrated under reduced pressure. The residue was purified by reverse phase flash column chromatography with CH₃CN/H₂O (68/32, v/v) to afford (3E)-3-[2-(dimethylamino)ethylidene]-1-{4-[(3-methyl-4-{[1,2,3,4]tetrazolo[1,5-a]pyridin-7-yloxy}phenyl)amino]pyrido[3,4-d]pyrimidin-6-yl}pyrrolidin-2-one (Compound 107) (5.2 mg, 2%) as a white solid. LCMS (ESI, m/z): [M+H]⁺=523.3. ¹H NMR (400 MHz, DMSO-d₆): δ 10.38 (s, 1H), 9.33 (d, J=7.6 Hz, 1H), 9.24 (s, 1H), 9.11 (s, 1H), 8.66 (s, 1H), 7.90-7.83 (m, 2H), 7.33 (d, J=7.6 Hz, 1H), 7.28 (d, J=8.8 Hz, 1H), 7.08 (d, J=2.4 Hz, 1H), 6.59-6.51 (m, 1H), 4.22-4.16 (m, 2H), 3.12 (d, J=6.8 Hz, 2H), 2.91-2.82 (m, 2H), 2.22 (s, 9H).

### Example S100: Synthesis of (3E)-3-[2-(dimethylamino)ethylidene]-1-{4-[(3-methyl-4-{[1,2,3,4]tetrazolo[1,5-a]pyridin-7-yloxy}phenyl)amino]pyrido[3,2-d]pyrimidin-6-yl}pyrrolidin-2-one (Compound 108)

### Step 1. Synthesis of 6-chloro-N-(3-methyl-4-{[1,2,3,4]tetrazolo[1,5-a]pyridin-7-yloxy}phenyl)pyrido[3,2-d]pyrimidin-4-amine

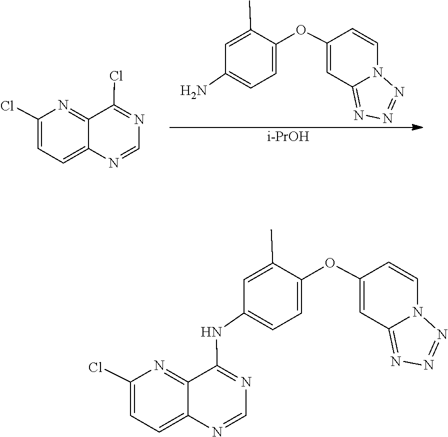 [SMILES: "i-PrOH" ; Clc1ccc2ncnc(Cl)c2n1 ; Cc1cc(N)ccc1Oc1ccn2nnnc2c1 ; Cc1cc(Nc2ncnc3ccc(Cl)nc23)ccc1Oc1ccn2nnnc2c1]

To a solution of 3-methyl-4-{[1,2,3,4]tetrazolo[1,5-a]pyridin-7-yloxy}aniline (200.0 mg, 0.83 mmol) in i-PrOH (4.0 mL) was added 4,6-dichloropyrido[3,2-d]pyrimidine (165.8 mg, crude) at room temperature. The resulting mixture was stirred at room temperature for 2 h. After the reaction was completed, the resulting mixture was concentrated under reduced pressure. The residue was purified by flash column chromatography with CH₂Cl₂/MeOH (8/1, v/v) to afford 6-chloro-N-(3-methyl-4-{[1,2,3,4]tetrazolo[1,5-a]pyridin-7-yloxy}phenyl)pyrido[3,2-d]pyrimidin-4-amine (220.0 mg, 65%) as a yellow solid. LCMS (ESI, m/z): [M+H]⁺=405.1.

### Step 2. Synthesis of (3E)-3-[2-(dimethylamino)ethylidene]-1-{4-[(3-methyl-4-{[1,2,3,4]tetrazolo[1,5-a]pyridin-7-yloxy}phenyl)amino]pyrido[3,2-d]pyrimidin-6-yl}pyrrolidin-2-one (Compound 108)

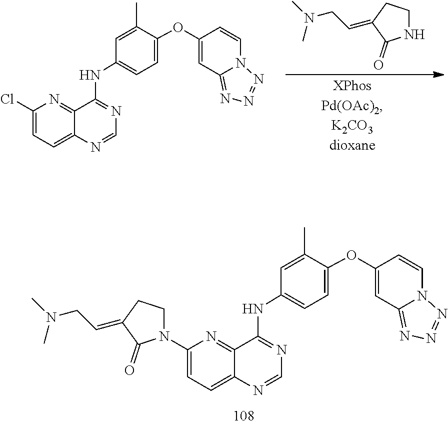 [SMILES: Cc1cc(Nc2ncnc3ccc(Cl)nc23)ccc1Oc1ccn2nnnc2c1 ; CN(C)C/C=C1\CCNC1=O ; Cc1cc(Nc2ncnc3ccc(N4CC/C(=C\CN(C)C)C4=O)nc23)ccc1Oc1ccn2nnnc2c1]

To a solution of 6-chloro-N-(3-methyl-4-{[1,2,3,4]tetrazolo[1,5-a]pyridin-7-yloxy}phenyl)pyrido[3,2-d]pyrimidin-4-amine (93.0 mg, 0.69 mmol) in dioxane (5.0 mL) was added (3E)-3-[2-(dimethylamino)ethylidene]pyrrolidin-2-one (35.4 mg, 0.23 mmol), K₂CO₃ (95.3 mg, 0.69 mmol), XPhos (21.9 mg, 0.05 mmol) and Pd(OAc)₂ (5.2 mg, 0.02 mmol) at room temperature under N₂. The resulting mixture was stirred at 100° C. for 1 h. After the reaction was completed, the reaction mixture was filtered. The filtrate was concentrated under reduced pressure. The residue was purified by flash column chromatography with CH₂Cl₂/MeOH (8/1, v/v) and then purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 30% B to 40% B in 8 min; Wave Length: 254 nm) to afford (3E)-3-[2-(dimethylamino)ethylidene]-1-{4-[(3-methyl-4-{[1,2,3,4]tetrazolo[1,5-a]pyridin-7-yloxy}phenyl)amino]pyrido[3,2-d]pyrimidin-6-yl}pyrrolidin-2-one (Compound 108) (23.6 mg, 19%) as a white solid. LCMS (ESI, m/z): [M+H]⁺=523.2. ¹H NMR (400 MHz, DMSO-d₆): δ 9.59 (s, 1H), 9.33 (d, J=7.6 Hz, 1H), 8.99 (d, J=9.6 Hz, 1H), 8.64 (s, 1H), 8.27 (d, J=9.2 Hz, 1H), 8.02-7.99 (m, 2H), 7.34-7.28 (m, 2H), 7.05 (s, 1H), 6.57 (s, 1H), 4.36-4.33 (m, 2H), 3.12-3.10 (m, 2H), 2.89-2.84 (m, 2H), 2.22-2.21 (m, 9H).

### Example S101: Synthesis of (E)-3-(2-(dimethylamino)ethylidene)-1-(4-((3-methyl-4-((1-(methyl-d3)-1H-benzo[d][1,2,3]triazol-5 yl)oxy)phenyl)amino)pyrido[3,2-d]pyrimidin-6-yl)pyrrolidin-2-one (Compound 117)

### Step 1. Synthesis of 5-bromo-1-(methyl-d3)-1H-benzo[d][1,2,3]triazole

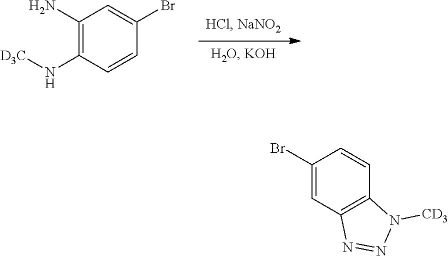 [SMILES: [2H]C([2H])([2H])Nc1ccc(Br)cc1N ; [2H]C([2H])([2H])n1nnc2cc(Br)ccc21]

To a solution of 4-bromo-N1-(methyl-d3)benzene-1,2-diamine (3.3 g, 16.17 mmol) in HCl (5.0 mL, 1 mol/L) was added a solution of NaNO₂ in H₂O (2.0 mL, 1 mol/L) at 0° C. under N₂. The resulting mixture was stirred at 0° C. for 4 h. After the reaction was completed, the mixture was basified to pH=8 with saturated KOH (aq.). The resulting mixture was extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) to afford 5-bromo-1-(methyl-d3)-1H-benzo[d][1,2,3]triazole (1.2 g, 34%) as a dark red solid. LCMS (ESI, m/z): [M+H]⁺=215.1.

### Step 2. Synthesis of 1-(methyl-d3)-1H-benzo[d][1,2,3]triazol-5-ol

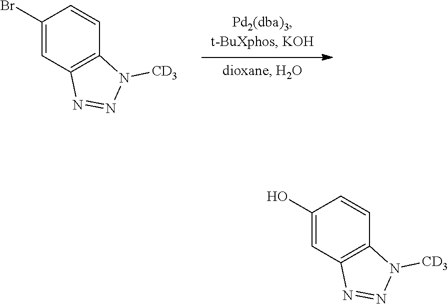 [SMILES: [2H]C([2H])([2H])n1nnc2cc(Br)ccc21 ; [2H]C([2H])([2H])n1nnc2cc(O)ccc21]

To a mixture of 5-bromo-1-(methyl-d3)-1H-benzo[d][1,2,3]triazole (1.0 g, 3.26 mmol) in dioxane (20.0 mL) and H₂O (4.0 mL) were added KOH (1.0 g, 18.60 mmol), t-BuXPhos (394.9 mg, 0.93 mmol) and Pd₂(dba)₃ (425.8 mg, 0.47 mmol) at room temperature under N₂O The resulting mixture was stirred at 100° C. for 4 h. After the reaction was completed, the resulting mixture was diluted with H₂O and extracted with ethyl acetate. The combined organic layer was dried over anhydrous sodium sulfate and filtered. The filtrate was concentrated under vacuum. The residue was purified by flash column chromatography with CH₂Cl₂/CH₃OH (10/1, v/v) to afford 1-(methyl-d3)-1H-benzo[d][1,2,3]triazol-5-ol (200.0 mg, 28%) as an brown solid. LCMS (ESI, m/z): [M+H]⁺=153.2.

### Step 3. Synthesis of 1-(methyl-d3)-5-(2-methyl-4-nitrophenoxy)-1H-benzo[d][1,2,3]triazole

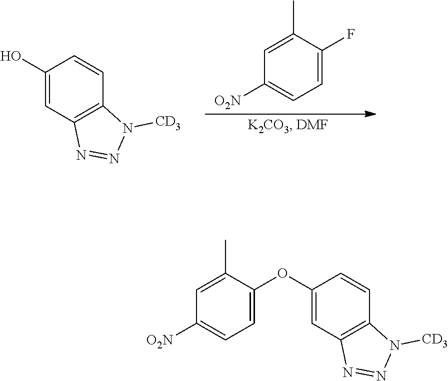 [SMILES: [2H]C([2H])([2H])n1nnc2cc(O)ccc21 ; Cc1cc([N+](=O)[O-])ccc1F ; [2H]C([2H])([2H])n1nnc2cc(Oc3ccc([N+](=O)[O-])cc3C)ccc21]

To a solution of 1-(methyl-d3)-1H-benzo[d][1,2,3]triazol-5-ol (180.0 mg, 1.18 mmol) in DMF (6.0 mL) was added 1-fluoro-2-methyl-4-nitrobenzene (183.5 mg, 1.18 mmol) and K₂CO₃ (326.9 mg, 2.37 mmol) at room temperature. The mixture was stirred at 50° C. for 1 h.

After the reaction was completed, the resulting mixture was diluted with H₂O and extracted with ethyl acetate. The combined organic layer was washed with brine, dried over anhydrous Na₂SO₄ and filtered. The filtrate was concentrated under reduced pressure. The residue was purified by flash column chromatography with CH₂Cl₂/CH₃OH (10/1, v/v) to afford 1-(methyl-d3)-5-(2-methyl-4-nitrophenoxy)-1H-benzo[d][1,2,3]triazole (390.0 mg, crude) as an brown solid. LCMS (ESI, m/z): [M+H]⁺=288.2.

### Step 4. Synthesis of 3-methyl-4-((1-(methyl-d3)-1H-benzo[d][1,2,3]triazol-5-yl)oxy)aniline

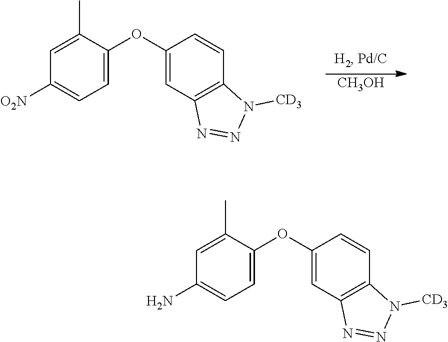 [SMILES: [2H]C([2H])([2H])n1nnc2cc(Oc3ccc([N+](=O)[O-])cc3C)ccc21 ; "CH3OH" ; [2H]C([2H])([2H])n1nnc2cc(Oc3ccc(N)cc3C)ccc21]

To a mixture of 1-(methyl-d3)-5-(2-methyl-4-nitrophenoxy)-1H-benzo[d][1,2,3]triazole (340.0 mg, 1.18 mmol) in methanol (5.0 mL) was added Pd/C (118.5 mg, 10%) at room temperature under N₂. The resulting mixture was stirred at room temperature for 1 h under H₂. After the reaction was completed, the resulting mixture was filtered. The filtrate was concentrated under reduced pressure. The residue was purified by flash column chromatography with CH₂Cl₂/CH₃OH (10/1, v/v) to afford 3-methyl-4-((1-(methyl-d3)-1H-benzo[d][1,2,3]triazol-5-yl)oxy)aniline (230.0 mg, 75%) as an brown solid. LCMS (ESI, m/z): [M+H]⁺=258.3.

### Step 5. Synthesis of 6-chloro-N-(3-methyl-4-((1-(methyl-d3)-1H-benzo[d][1,2,3]triazol-5-yl)oxy)phenyl)pyrido[3,2-d]pyrimidin-4-amine

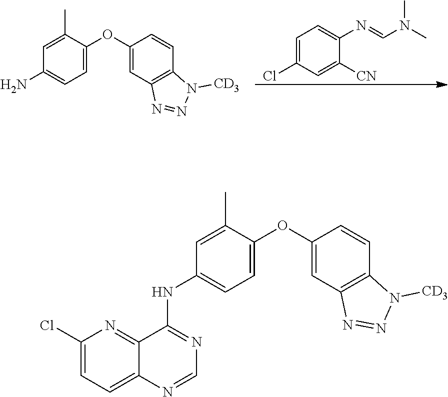 [SMILES: [2H]C([2H])([2H])n1nnc2cc(Oc3ccc(N)cc3C)ccc21 ; CN(C)/C=N/c1ccc(Cl)cc1C#N ; [2H]C([2H])([2H])n1nnc2cc(Oc3ccc(Nc4ncnc5ccc(Cl)nc45)cc3C)ccc21]

To a mixture of 3-methyl-4-((1-(methyl-d3)-1H-benzo[d][1,2,3]triazol-5-yl)oxy)aniline (180.0 mg, 0.70 mmol) in CH₃COOH (5.0 mL) were added (E)-N′-(6-chloro-2-cyanopyridin-3-yl)-N,N-dimethylformimidamide (145.9 mg, 0.70 mmol) at room temperature.

The resulting mixture was stirred at 85° C. for 1 h. After the reaction was completed, the resulting mixture was concentrated under reduced pressure. The residue was purified by flash column chromatography with CH₂Cl₂/CH₃OH (10/1, v/v) to afford 6-chloro-N-(3-methyl-4-((1-(methyl-d3)-1H-benzo[d][1,2,3]triazol-5-yl)oxy)phenyl)pyrido[3,2-d]pyrimidin-4-amine (230.0 mg, 78%) as a white solid. LCMS (ESI, m/z): [M+H]⁺=421.1.

### Step 6. Synthesis of (E)-3-(2-(dimethylamino)ethylidene)-1-(4-((3-methyl-4-((1-(methyl-d3)-1H-benzo[d][1,2,3]triazol-5-yl)oxy)phenyl)amino)pyrido[3,2-d]pyrimidin-6-yl)pyrrolidin-2-one (Compound 117)

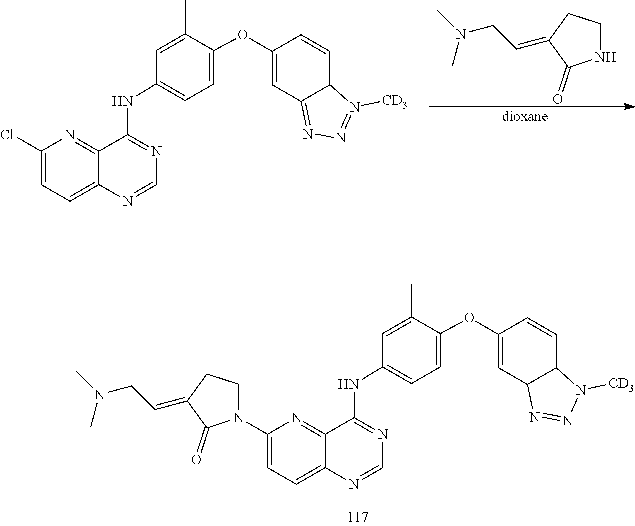

To a mixture of 6-chloro-N-(3-methyl-4-((1-(methyl-d3)-1H-benzo[d][1,2,3]triazol-5-yl)oxy)phenyl)pyrido[3,2-d]pyrimidin-4-amine (150.0 mg, 0.36 mmol) and (E)-3-(2-(dimethylamino)ethylidene)pyrrolidin-2-one (82.4 mg, 0.53 mmol) in dioxane (15.0 mL) were added K₂CO₃ (147.8 mg, 1.07 mmol), XPhos (33.9 mg, 0.07 mmol) and Pd(OAc)₂ (8.0 mg, 0.04 mmol) at room temperature. The resulting mixture was stirred at 100° C. for 12 h under N₂. After the reaction was completed, the resulting mixture was diluted with H₂O and extracted with ethyl acetate. The combined organic layer was washed with brine and dried over anhydrous Na₂SO₄. After filtration, the filtrate was concentrated under reduced pressure. The residue was purified by flash column chromatography with CH₂Cl₂/CH₃OH (10/1, v/v) and then purified by Prep-HPLC with the following conditions (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 44% B to 50% B in 8 min; Wave Length: 254 nm) to afford (E)-3-(2-(dimethylamino)ethylidene)-1-(4-((3-methyl-4-((1-(methyl-d3)-1H-benzo[d][1,2,3]triazol-5-yl)oxy)phenyl)amino)pyrido[3,2-d]pyrimidin-6-yl)pyrrolidin-2-one (Compound 117) (33.8 mg, 17%) as a white solid. LCMS (ESI, m/z): [M+H]⁺=539.3. ¹H NMR (400 MHz, DMSO-d₆): δ 9.57 (s, 1H), 8.99-8.97 (m, 1H), 8.60 (s, 1H), 8.26 (d, J=9.2 Hz, 1H), 7.91-7.86 (m, 3H), 7.37-7.35 (m, 1H), 7.28 (s, 1H), 7.04 (d, J=8.4 Hz, 1H), 6.62-6.55 (m, 1H), 4.37-4.33 (m, 2H), 3.11 (d, J=6.4 Hz, 2H), 2.91-2.84 (m, 2H), 2.25-2.21 (m, 9H).

### Example S102: Synthesis of 6-[(3E)-3 [2-(dimethylamino)ethylidene]-2-oxopyrrolidin-1 yl]-4-({3-methyl-4-[(1-methyl-1,3-benzodiazol-5-yl)oxy]phenyl}amino)-1,5-naphthyridine-3-carbonitrile (Compound 124)

### Step 1. Synthesis of Ethyl (2E)-3-[(6-chloropyridin-3-yl)amino]-2-cyanoprop-2-enoate

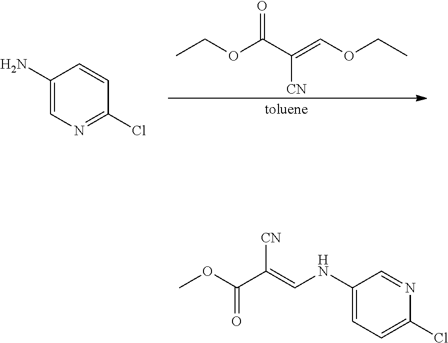 [SMILES: Nc1ccc(Cl)nc1 ; CCO/C=C(\C#N)C(=O)OCC ; COC(=O)/C(C#N)=C/Nc1ccc(Cl)nc1]

To a solution of 6-chloropyridin-3-amine (2.0 g, 15.55 mmol) in toluene (50.0 mL) was added ethyl (2E)-2-cyano-3-ethoxyprop-2-enoate (2.6 g, 15.55 mmol) at room temperature. The resulting mixture was stirred at 100 C for 4 h. After the reaction was completed, the mixture was filtered. The solid was washed with CH₃CN and collected to afford ethyl (2E)-3-[(6-chloropyridin-3-yl)amino]-2-cyanoprop-2-enoate (2.5 g, crude) as a white solid. LCMS (ESI, m/z): [M+H]⁺=252.0.

### Step 2. Synthesis of 6-chloro-4-oxo-1H-1,5-naphthyridine-3-carbonitrile

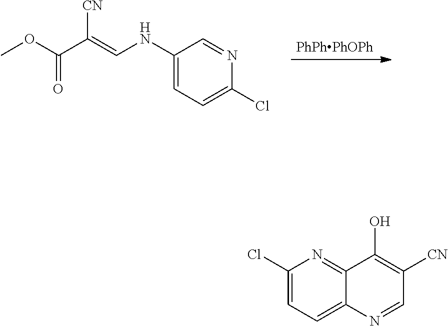 [SMILES: COC(=O)/C(C#N)=C/Nc1ccc(Cl)nc1 ; N#Cc1cnc2ccc(Cl)nc2c1O]

A solution of ethyl (2E)-3-[(6-chloropyridin-3-yl)amino]-2-cyanoprop-2-enoate (3.0 g, 11.92 mmol) in PhPh·PhOPh (90.0 mL) was stirred at 240° C. for 8 h. After the reaction was completed, the resulting mixture was diluted with n-hexane and then filtered. The solid was washed with EtOH and collected to afford 6-chloro-4-oxo-1H-1,5-naphthyridine-3-carbonitrile (0.2 g, crude) as a brown solid. LCMS (ESI, m/z): [M+H]⁺=206.0.

### Step 3. Synthesis of 4,6-dichloro-1,5-naphthyridine-3-carbonitrile

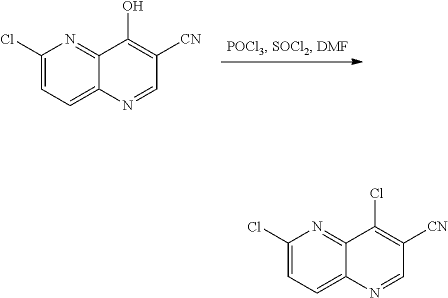 [SMILES: N#Cc1cnc2ccc(Cl)nc2c1O ; N#Cc1cnc2ccc(Cl)nc2c1Cl]

To a solution of 6-chloro-4-oxo-1H-1,5-naphthyridine-3-carbonitrile (200.0 mg, 0.97 mmol) in SOCl₂/POCl₃ (5.0/5.0 mL) was added DMF (0.02 mL) at room temperature. The resulting mixture was stirred at 80° C. for 1 h. After the reaction was completed, the resulting mixture was concentrated under reduced pressure to afford 4,6-dichloro-1,5-naphthyridine-3-carbonitrile (200.0 mg, crude) as a brown solid. LCMS (ESI, m/z): [M+H]⁺=224.0.

### Step 4. Synthesis of 6-chloro-4-({3-methyl-4-[(1-methyl-1,3-benzodiazol-5-yl)oxy]phenyl}amino)-1,5-naphthyridine-3-carbonitrile

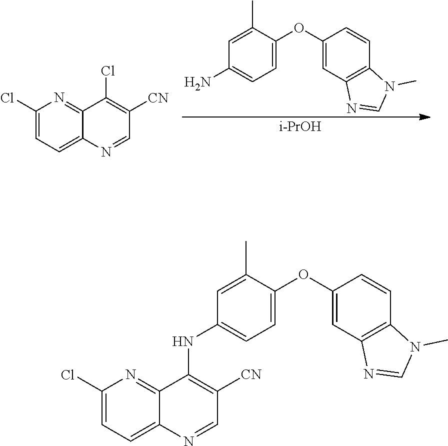 [SMILES: "i-PrOH" ; N#Cc1cnc2ccc(Cl)nc2c1Cl ; Cc1cc(N)ccc1Oc1ccc2c(c1)ncn2C ; Cc1cc(Nc2c(C#N)cnc3ccc(Cl)nc23)ccc1Oc1ccc2c(c1)ncn2C]

To a solution of 4,6-dichloro-1,5-naphthyridine-3-carbonitrile (200.0 mg, 0.89 mmol) in i-PrOH (10.0 mL) was added 3-methyl-4-[(1-methyl-1,3-benzodiazol-5-yl)oxy]aniline (226.0 mg, 0.89 mmol) at 0° C. under N₂. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced. The residue was purified by flash column chromatography with CH₂Cl₂/MeOH (90/10, v/v) to afford 6-chloro-4-({3-methyl-4-[(1-methyl-1,3-benzodiazol-5-yl)oxy]phenyl}amino)-1,5-naphthyridine-3-carbonitrile (100.0 mg, 25%) as a brown yellow solid. LCMS (ESI, m/z): [M+H]⁺=441.1.

### Step 5. Synthesis of 6-[(3E)-3-[2-(dimethylamino)ethylidene]-2-oxopyrrolidin-1-yl]-4-({3-methyl-4-[(1-methyl-1,3-benzodiazol-5-yl)oxy]phenyl}amino)-1,5-naphthyridine-3-carbonitrile (Compound 124)

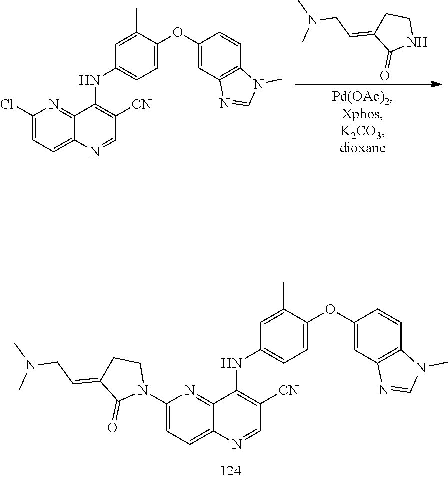

To a solution of 6-chloro-4-({3-methyl-4-[(1-methyl-1,3-benzodiazol-5-yl)oxy]phenyl}amino)-1,5-naphthyridine-3-carbonitrile (100.0 mg, 0.23 mmol) in 1,4-dioxane (5.0 mL) was added (3E)-3-[2-(dimethylamino)ethylidene]pyrrolidin-2-one (70.0 mg, 0.45 mmol), Pd(OAc)₂ (5.1 mg, 0.02 mmol), X-Phos (21.6 mg, 0.05 mmol) and K₂CO₃ (62.7 mg, 0.45 mmol) at room temperature under N₂. The resulting mixture was stirred at 100° C. for 1 h. After the reaction was completed, the resulting mixture was filtered. The filtrate was concentrated under reduced pressure. The residue was purified by flash column chromatography with CH₂Cl₂/MeOH (85/15, v/v) and then purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 30% B to 40% B in 8 min; Wave Length: 254 nm) to afford 6-[(3E)-3-[2-(dimethylamino)ethylidene]-2-oxopyrrolidin-1-yl]-4-({3-methyl-4-[(1-methyl-1,3-benzodiazol-5-yl)oxy]phenyl}amino)-1,5-naphthyridine-3-carbonitrile (Compound 124) (24.6 mg, 19%) as an off-white solid. LCMS (ESI, m/z): [M+H]⁺=559.3. ¹H NMR (400 MHz, DMSO-d₆): δ 9.73 (s, 1H), 8.95 (d, J=9.2 Hz, 1H), 8.51 (s, 1H), 8.35 (d, J=9.2 Hz, 1H), 8.18 (s, 1H), 7.57 (d, J=8.8 Hz, 1H), 7.40 (d, J=2.0 Hz, 1H), 7.27-7.25 (m, 1H), 7.17 (d, J=2.4 Hz, 1H), 7.04-7.01 (m, 1H), 6.94 (d, J=8.4 Hz, 1H), 6.59-6.55 (m, 1H), 4.33-4.29 (m, 2H), 3.85 (s, 3H), 3.14-3.07 (m, 2H), 2.87-2.81 (m, 2H), 2.26 (s, 3H), 2.21 (s, 6H).

### Example S103: Synthesis of (3E)-1-(4-{[4-(1H-1,3-benzodiazol-5-yloxy)-3-methylphenyl]amino}pyrido[3,2-d]pyrimidin-6-yl)-3-[2-(dimethylamino)ethylidene]pyrrolidin-2-one (Compound 118)

### Step 1. Synthesis of Tert-butyl 5-hydroxy-1,3-benzodiazole-1-carboxylate

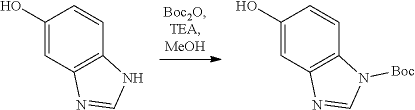 [SMILES: Oc1ccc2[nH]cnc2c1 ; CC(C)(C)OC(=O)n1cnc2cc(O)ccc21]

To a mixture of 1H-1,3-benzodiazol-5-ol (500.0 mg, 3.72 mmol) in MeOH (10.0 mL) was added TEA (377.2 mg, 3.72 mmol) and Boc₂O (976.2 mg, 4.47 mmol) at room temperature. The resulting mixture was stirred at room temperature for 16 h. After the reaction was completed, the resulting mixture was concentrated under reduced pressure. The residue was purified by flash column chromatography with petroleum ether/ethyl acetate (5/1, v/v) to afford tert-butyl 5-hydroxy-1,3-benzodiazole-1-carboxylate (350.0 mg, 40%) as a white solid. LCMS (ESI, m/z): [M+H]⁺=235.1.

### Step 2. Synthesis of Tert-butyl 5-(2-methyl-4-nitrophenoxy)-1,3-benzodiazole-1-carboxylate

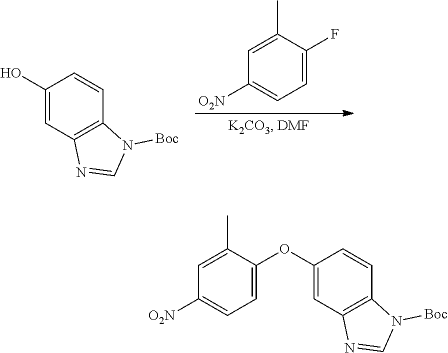 [SMILES: CC(C)(C)OC(=O)n1cnc2cc(O)ccc21 ; Cc1cc([N+](=O)[O-])ccc1F ; Cc1cc([N+](=O)[O-])ccc1Oc1ccc2c(c1)ncn2C(=O)OC(C)(C)C]

To a mixture of tert-butyl 5-hydroxy-1,3-benzodiazole-1-carboxylate (350.0 mg, 1.49 mmol) in DMF (5.0 mL) was added 1-fluoro-2-methyl-4-nitrobenzene (231.7 mg, 1.49 mmol) and K₂CO₃ (825.9 mg, 5.97 mmol) at room temperature. The resulting mixture was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (1/1, v/v) to afford tert-butyl 5-(2-methyl-4-nitrophenoxy)-1,3-benzodiazole-1-carboxylate (220. mg, 39%) as a colorless oil. LCMS (ESI, m/z): [M+H]⁺=370.1.

### Step 3. Synthesis of Tert-butyl 5-(4-amino-2-methylphenoxy)-1,3-benzodiazole-1-carboxylate

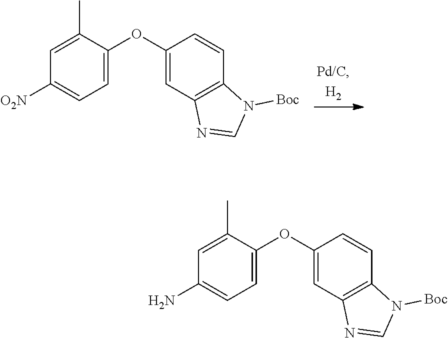 [SMILES: Cc1cc([N+](=O)[O-])ccc1Oc1ccc2c(c1)ncn2C(=O)OC(C)(C)C ; Cc1cc(N)ccc1Oc1ccc2c(c1)ncn2C(=O)OC(C)(C)C]

To a solution of tert-butyl 5-(2-methyl-4-nitrophenoxy)-1,3-benzodiazole-1-carboxylate (200.0 mg, 0.54 mmol) in MeOH (2.0 mL) was added Pd/C (60.0 mg, 10%) at room temperature under N₂. The mixture was stirred at room temperature for 2 h under H₂. After the reaction was completed, the reaction mixture was filtered. The filtrate was concentrated under vacuum. The residue was purified by flash column chromatography with petroleum ether/ethyl acetate (1/1, v/v) to afford tert-butyl 5-(4-amino-2-methylphenoxy)-1,3-benzodiazole-1-carboxylate (150.0 mg, 81%) as a white solid. LCMS (ESI, m/z): [M+H]⁺=340.2.

### Step 4. Synthesis of Tert-butyl 5-[4-({6-chloropyrido[3,2-d]pyrimidin-4-yl}amino)-2-methylphenoxy]-1,3-benzodiazole-1-carboxylate

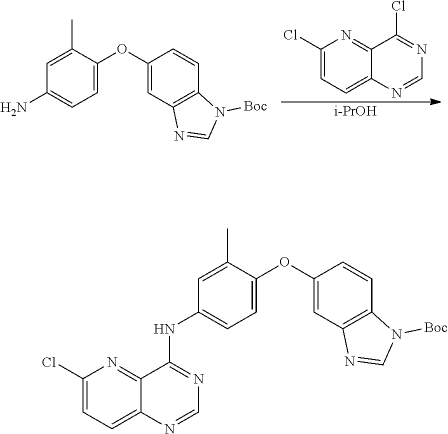 [SMILES: "i-PrOH" ; Cc1cc(N)ccc1Oc1ccc2c(c1)ncn2C(=O)OC(C)(C)C ; Clc1ccc2ncnc(Cl)c2n1 ; Cc1cc(Nc2ncnc3ccc(Cl)nc23)ccc1Oc1ccc2c(c1)ncn2C(=O)OC(C)(C)C]

To a solution of tert-butyl 5-(4-amino-2-methylphenoxy)-1,3-benzodiazole-1-carboxylate (150.0 mg, 0.44 mmol) in i-PrOH (3.0 mL) was added 4,6-dichloropyrido[3,2-d]pyrimidine (88.4 mg, 0.44 mmol) at room temperature. The resulting mixture was stirred at room temperature for 2 h. After the reaction was completed, the resulting mixture was concentrated under vacuum, The residue was purified by flash column chromatography with CH₂Cl₂/MeOH (10/1, v/v) to afford tert-butyl 5-[4-({6-chloropyrido[3,2-d]pyrimidin-4-yl}amino)-2-methylphenoxy]-1,3-benzodiazole-1-carboxylate (140.0 mg, 62%) as a yellow solid. LCMS (ESI, m/z): [M+H]⁺=503.2.

### Step 5. Synthesis of Tert-butyl 5-[4-({6-[(3E)-3-[2-(dimethylamino)ethylidene]-2-oxopyrrolidin-1-yl]pyrido[3,2-d]pyrimidin-4-yl}amino)-2-methylphenoxy]-1,3-benzodiazole-1-carboxylate

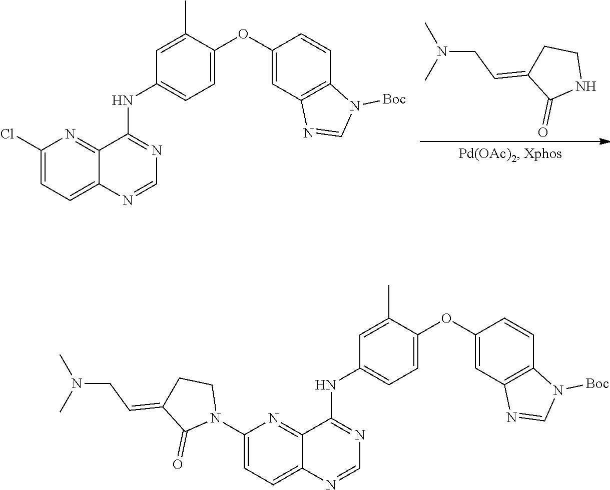

To a mixture of tert-butyl 5-[4-({6-chloropyrido[3,2-d]pyrimidin-4-yl}amino)-2-methylphenoxy]-1,3-benzodiazole-1-carboxylate (130.0 mg, 0.25 mmol) in 1,4-dioxane (4.0 mL) was added (3E)-3-[2-(dimethylamino)ethylidene]pyrrolidin-2-one (39.8 g, 0.25 mmol), Cs₂CO₃ (252.6 mg, 0.77 mmol), Xphos (24.6 mg, 0.05 mmol) and Pd(OAc)₂ (5.8 mg, 0.02 mmol) at room temperature under N₂. The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford tert-butyl 5-[4-({6-[(3E)-3-[2-(dimethylamino)ethylidene]-2-oxopyrrolidin-1-yl]pyrido[3,2-d]pyrimidin-4-yl}amino)-2-methylphenoxy]-1,3-benzodiazole-1-carboxylate (50.0 mg, 31%) as a yellow solid. LCMS (ESI, m/z): [M+H]⁺=621.3.

### Step 6. Synthesis of (3E)-1-(4-{[4-(1H-1,3-benzodiazol-5-yloxy)-3-methylphenyl]amino}pyrido[3,2-d]pyrimidin-6-yl)-3-[2-(dimethylamino)ethylidene]pyrrolidin-2-one (Compound 118)

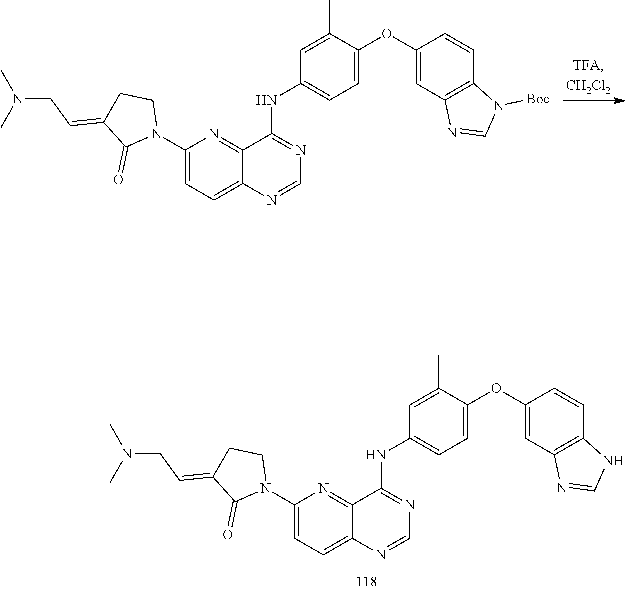 [SMILES: Cc1cc(Nc2ncnc3ccc(N4CC/C(=C\CN(C)C)C4=O)nc23)ccc1Oc1ccc2c(c1)ncn2C(=O)OC(C)(C)C ; Cc1cc(Nc2ncnc3ccc(N4CC/C(=C\CN(C)C)C4=O)nc23)ccc1Oc1ccc2[nH]cnc2c1]

To a solution of tert-butyl 5-[4-({6-[(3E)-3-[2-(dimethylamino)ethylidene]-2-oxopyrrolidin-1-yl]pyrido[3,2-d]pyrimidin-4-yl}amino)-2-methylphenoxy]-1,3-benzodiazole-1-carboxylate (50.0 mg, 0.08 mmol) in CH₂Cl₂ (2.0 mL) was added TFA (0.6 mL) at room temperature. The resulting mixture was stirred at room temperature for 1 h. After the reaction was completed, the mixture was evaporated in vacuo. The residue was purified by Prep-HPLC with the following conditions: (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 38% B to 50% B in 8 min; Wave Length: 254 nm) to afford (3E)-1-(4-{[4-(1H-1,3-benzodiazol-5-yloxy)-3-methylphenyl]amino}pyrido[3,2-d]pyrimidin-6-yl)-3-[2-(dimethylamino)ethylidene]pyrrolidin-2-one (Compound 118) (16.4 mg, 38%) as a yellow solid. LCMS (ESI, m/z): [M+H]⁺=521.3. ¹H NMR (400 MHz, DMSO-d₆): δ 12.44-12.26 (m, 1H), 9.51 (s, 1H), 8.97 (d, J=9.2 Hz, 1H), 8.59 (s, 1H), 8.25-8.15 (m, 2H), 7.84-7.51 (m, 3H), 7.11-6.91 (m, 3H), 6.58-6.55 (m, 1H), 4.35-4.32 (m, 2H), 3.11 (d, J=6.8 Hz, 2H), 2.89-2.84 (m, 2H), 2.26 (s, 3H), 2.21 (s, 6H).

### Example S104: Synthesis of (3E)-3 [2-(dimethylamino)ethylidene]-1-(4-{[3-fluoro-4-(pyridin-3-yloxy)phenyl]amino}pyrido[3,4-d]pyrimidin-6-yl)pyrrolidin-2-one (Compound 125)

### Step 1. Synthesis of 6-chloro-N-[3-fluoro-4-(pyridin-3-yloxy)phenyl]pyrido[3,4-d]pyrimidin-4-amine

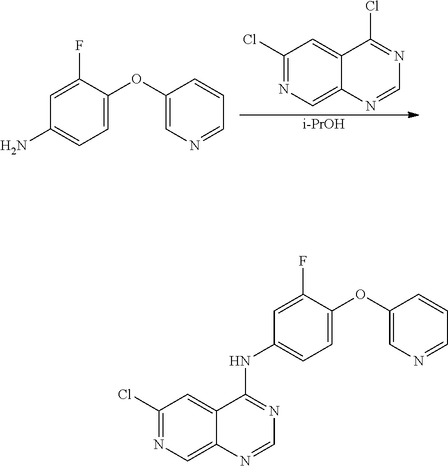 [SMILES: "i-PrOH" ; Nc1ccc(Oc2cccnc2)c(F)c1 ; Clc1cc2c(Cl)ncnc2cn1 ; Fc1cc(Nc2ncnc3cnc(Cl)cc23)ccc1Oc1cccnc1]

To a solution of 3-fluoro-4-(pyridin-3-yloxy)aniline (300.0 mg, 1.47 mmol) in i-PrOH (6.0 mL) was added 4,6-dichloropyrido[3,4-d]pyrimidine (293.9 mg, 1.47 mmol) at 0° C. under N₂. The resulting mixture was stirred at room temperature for 1 h. After the reaction was completed, the reaction mixture was concentrated under reduced pressure. The residue was purified by flash column chromatography with CH₂Cl₂/CH₃OH (85/15, v/v) to afford 6-chloro-N-[3-fluoro-4-(pyridin-3-yloxy)phenyl]pyrido[3,4-d]pyrimidin-4-amine (600.0 mg, 88%) as a yellow solid. LCMS (ESI, m/z): [M+H]⁺=368.1.

### Step 2. Synthesis of (3E)-3-[2-(dimethylamino)ethylidene]-1-(4-{[3-fluoro-4-(pyridin-3-yloxy)phenyl]amino}pyrido[3,4-d]pyrimidin-6-yl)pyrrolidin-2-one (Compound 125)

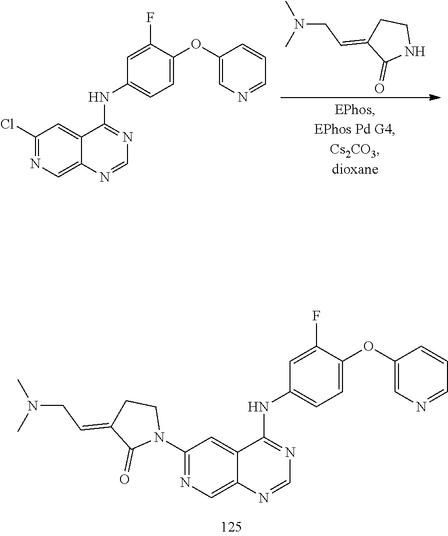

To a solution of 6-chloro-N-[3-fluoro-4-(pyridin-3-yloxy)phenyl]pyrido[3,4-d]pyrimidin-4-amine (300.0 mg, 0.82 mmol) in dioxane (10.0 mL) was added (3E)-3-[2-(dimethylamino)ethylidene]pyrrolidin-2-one (125.8 mg, 0.82 mmol), Cs₂CO₃ (797.3 mg, 2.45 mmol), EPhos Pd G4 (74.9 mg, 0.08 mmol) and EPhos (87.3 mg, 0.16 mmol) at room temperature under N₂. The resulting mixture was stirred at 100° C. for 2 h. After the reaction was completed, the resulting mixture was diluted with water and extracted with ethyl acetate. The combined organic layer was washed with brine, dried over anhydrous sodium sulfate and filtered. The filtrate was concentrated under reduced pressure. The residue was purified by reverse phase flash chromatography with CH₃OH/H₂O (90/10, v/v) and then purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 33% B to 38% B in 8 min, Wave Length: 254 nm) to afford (3E)-3-[2-(dimethylamino)ethylidene]-1-(4-{[3-fluoro-4-(pyridin-3-yloxy)phenyl]amino}pyrido[3,4-d]pyrimidin-6-yl)pyrrolidin-2-one (Compound 125) (28.5 mg, 7%) as a white solid. LCMS (ESI, m/z): [M+H]⁺=486.2. ¹H NMR (400 MHz, DMSO-d₆): δ 10.42 (s, 1H), 9.21 (s, 1H), 9.15-9.11 (m, 1H), 8.69 (s, 1H), 8.43-8.30 (m, 2H), 8.12-8.09 (m, 1H), 7.73-7.65 (m, 1H), 7.46-7.33 (m, 3H), 6.55-6.52 (m, 1H), 4.19-4.16 (m, 2H), 3.13-3.08 (m, 2H), 2.89-2.84 (m, 2H), 2.21 (s, 6H).

### Example S105: Synthesis of (3E)-3 [2-(dimethylamino)ethylidene]-1-(4-{[3-fluoro-4-(pyridin-3-yloxy)phenyl]amino}pyrido[3,2-d]pyrimidin-6-yl)pyrrolidin-2-one (Compound 126)

### Step 1. Synthesis of 3-(2-fluoro-4-nitrophenoxy)pyridine

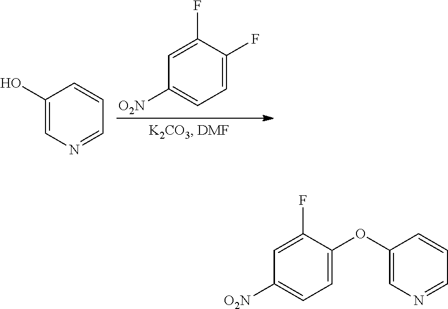

To a solution of 3-hydroxypyridine (2.0 g, 21.03 mmol) in DMF (300.0 mL) was added 1,2-difluoro-4-nitrobenzene (3.4 g, 21.03 mmol) and K₂CO₃ (11.6 g, 84.12 mmol) at room temperature. The resulting mixture was stirred at 50° C. for 16 h. After the reaction was completed, the resulting mixture was diluted with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (50/50, v/v) to afford 3-(2-fluoro-4-nitrophenoxy)pyridine (6.5 g, 92%) as a yellow oil. LCMS (ESI, m/z): [M+H]⁺=235.0.

### Step 2. Synthesis of 3-fluoro-4-(pyridin-3-yloxy)aniline

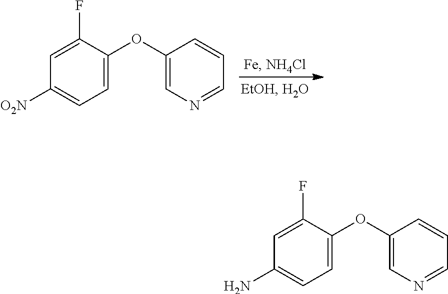 [SMILES: O=[N+]([O-])c1ccc(Oc2cccnc2)c(F)c1 ; Nc1ccc(Oc2cccnc2)c(F)c1]

To a solution of 3-(2-fluoro-4-nitrophenoxy)pyridine (6.0 g, 25.62 mmol) in EtOH/H₂O (200.0/200.0 mL) was added NH₄Cl (6.9 g, 128.11 mmol) and Fe (7.2 g, 128.11 mmol) at room temperature. The resulting mixture was stirred at 80° C. for 2 h. After the reaction was completed, the resulting mixture was filtered. The filtrate was concentrated under reduced pressure. The residue was purified by flash column chromatography with petroleum ether/ethyl acetate (60/40, v/v) to afford 3-fluoro-4-(pyridin-3-yloxy)aniline (3.0 g, 57%) as a white solid. LCMS (ESI, m/z): [M+H]⁺=205.1.

### Step 3. Synthesis of 6-chloro-N-[3-fluoro-4-(pyridin-3-yloxy)phenyl]pyrido[3,2-d]pyrimidin-4-amine

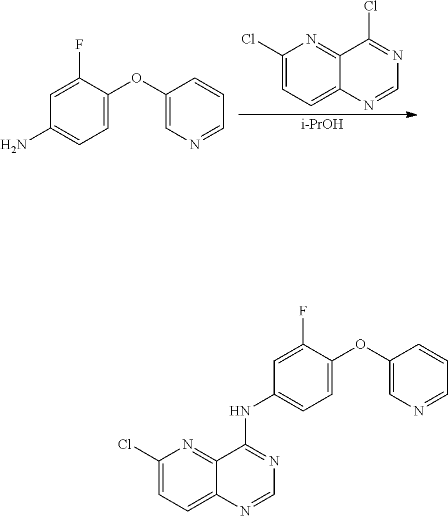 [SMILES: "i-PrOH" ; Nc1ccc(Oc2cccnc2)c(F)c1 ; Clc1ccc2ncnc(Cl)c2n1 ; Fc1cc(Nc2ncnc3ccc(Cl)nc23)ccc1Oc1cccnc1]

To a solution of 3-fluoro-4-(pyridin-3-yloxy)aniline (300.0 mg, 1.47 mmol) in i-PrOH (6.0 mL) was added 4,6-dichloropyrido[3,2-d]pyrimidine (293.9 mg, 1.47 mmol) at 0° C. under N₂. The resulting mixture was stirred at room temperature for 1 h. After the reaction was completed, the filtrate was concentrated under reduced pressure. The residue was purified by flash column chromatography with CH₂Cl₂/CH₃OH (85/15, v/v) to afford 6-chloro-N-[3-fluoro-4-(pyridin-3-yloxy)phenyl]pyrido[3,2-d]pyrimidin-4-amine (600.0 mg, 88%) as a yellow solid. LCMS (ESI, m/z): [M+H]⁺=368.1.

### Step 4. Synthesis of (3E)-3-[2-(dimethylamino)ethylidene]-1-(4-{[3-fluoro-4-(pyridin-3-yloxy)phenyl]amino}pyrido[3,2-d]pyrimidin-6-yl)pyrrolidin-2-one (Compound 126)

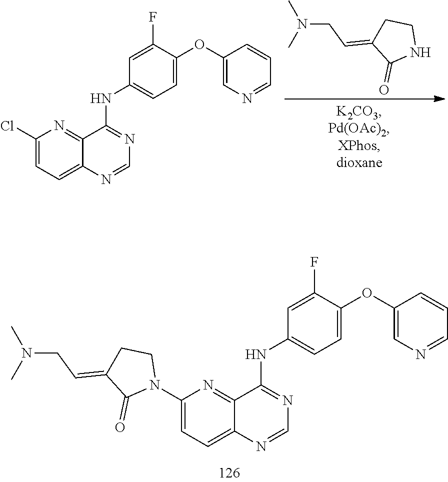

To a solution of 6-chloro-N-[3-fluoro-4-(pyridin-3-yloxy)phenyl]pyrido[3,2-d]pyrimidin-4-amine (300.0 mg, 0.82 mmol) in dioxane (10.0 mL) was added (3E)-3-[2-(dimethylamino)ethylidene]pyrrolidin-2-one (125.8 mg, 0.82 mmol), K₂CO₃ (338.2 mg, 2.45 mmol), Pd(OAc)₂ (18.0 mg, 0.08 mmol) and XPhos (77.8 mg, 0.16 mmol) at room temperature under N₂. The resulting mixture was stirred at 100° C. for 2 h. After the reaction was completed, the resulting mixture was diluted with water and extracted with ethyl acetate. The combined organic layer was washed with brine, dried over anhydrous sodium sulfate and filtered. The filtrate was concentrated under reduced pressure. The residue was purified by reverse phase flash chromatography with CH₃OH/H₂O (90/10, v/v) and then purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 35% B to 40% B in 8 min, Wave Length: 254 nm) to afford (3E)-3-[2-(dimethylamino)ethylidene]-1-(4-{[3-fluoro-4-(pyridin-3-yloxy)phenyl]amino}pyrido[3,2-d]pyrimidin-6-yl)pyrrolidin-2-one (Compound 126) (37.3 mg, 9%) as a white solid. LCMS (ESI, m/z): [M+H]⁺=486.1. ¹H NMR (400 MHz, DMSO-d₆): δ 9.66 (s, 1H), 8.99 (d, J=9.2 Hz, 1H), 8.67 (s, 1H), 8.42-8.36 (m, 2H), 8.29-8.22 (m, 2H), 7.87 (d, J=8.8 Hz, 1H), 7.45-7.35 (m, 3H), 6.62-6.57 (m, 1H), 4.36-4.33 (m, 2H), 3.12-3.10 (m, 2H), 2.91-2.86 (m, 2H), 2.21 (s, 6H).

### Example S106: Synthesis of 1-[4-({3-methyl-4-[(1-methyl-1,3-benzodiazol-5-yl)oxy]phenyl}amino)pyrido[3,4-d]pyrimidin-6-yl]-3-methylidenepiperidin-2-one (Compound 127)

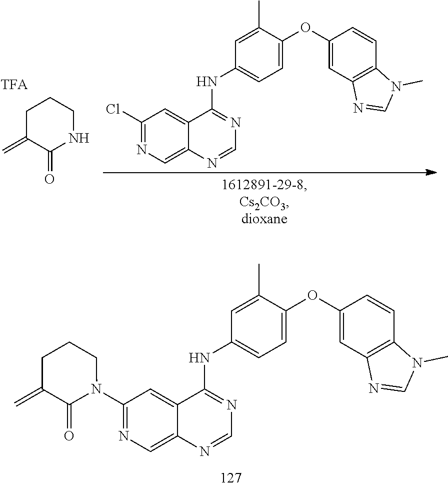

A mixture of 3-methylidenepiperidin-2-one trifluoroacetate (239.5 mg, crude), 6-chloro-N-{3-methyl-4-[(1-methyl-1,3-benzodiazol-5-yl)oxy]phenyl}pyrido[3,4-d]pyrimidin-4-amine (300.0 mg, 0.72 mmol), Pd-PEPPSI-IPentCl 2-methylpyridine (o-picoline) (121.7 mg, 0.14 mmol) and Cs₂CO₃ (703.43 mg, 2.16 mmol) in dioxane (20.0 mL) was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) and then purified by Prep-HPLC with the following conditions (Column: XBridge Prep Phenyl OBD Column, 19×250 mm, 5 μm; Mobile Phase A: Water (10 mmol/L NH₄HCO₃), Mobile Phase B: MeOH-HPLC; Flow rate: 25 mL/min; Gradient: 72% B to 77% B in 12 min, 254 nm) to afford 1-[4-({3-methyl-4-[(1-methyl-1,3-benzodiazol-5-yl)oxy]phenyl}amino)pyrido[3,4-d]pyrimidin-6-yl]-3-methylidenepiperidin-2-one (Compound 127) (3.8 mg, 1%) as a yellow solid. LCMS (ESI, m/z): [M+H]⁺=492.3. ¹H NMR (400 MHz, DMSO-d₆): δ 10.13 (s, 1H), 9.09 (s, 1H), 8.70-8.66 (m, 2H), 8.18 (s, 1H), 7.78 (s, 1H), 7.67-7.57 (m, 2H), 7.13 (s, 1H), 7.02 (d, J=8.0 Hz, 1H), 6.87 (d, J=8.4 Hz, 1H), 6.21 (s, 1H), 5.54 (s, 1H), 4.03-4.00 (m, 2H), 3.85 (s, 3H), 2.76-2.72 (m, 2H), 2.27 (s, 3H), 2.08-1.98 (m, 2H).

### Example S107: Synthesis of (E)-1-(4-((4-([1,2,4]triazolo[1,5-a]pyridin-7-yloxy)-3-methylphenyl)amino)pyrido[3,4-d]pyrimidin-6-yl)-3-(2-(azetidin-1 yl)ethylidene)pyrrolidin-2-one (Compound 102)

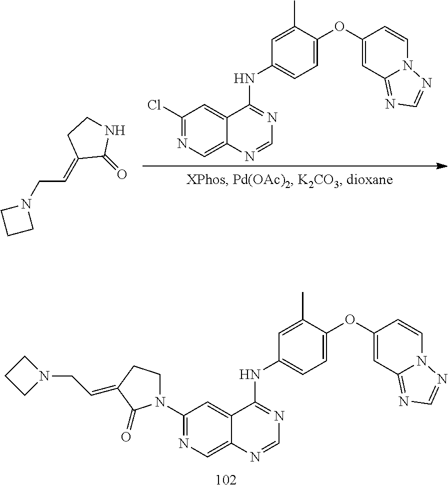 [SMILES: O=C1NCC/C1=C\CN1CCC1 ; Cc1cc(Nc2ncnc3cnc(Cl)cc23)ccc1Oc1ccn2ncnc2c1 ; Cc1cc(Nc2ncnc3cnc(N4CC/C(=C\CN5CCC5)C4=O)cc23)ccc1Oc1ccn2ncnc2c1]

To a solution of (E)-3-(2-(azetidin-1-yl)ethylidene)pyrrolidin-2-one (150.0 mg, 0.90 mmol) in dioxane (5.0 mL) was added N-(4-([1,2,4]triazolo[1,5-a]pyridin-7-yloxy)-3-methylphenyl)-6-chloropyrido[3,4-d]pyrimidin-4-amine (150.0 mg, 0.37 mmol), K₂CO₃ (1247.1 mg, 9.02 mmol), XPhos (86.0 mg, 0.18 mmol) and Pd(OAc)₂ (20.2 mg, 0.09 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: (Column: XSelect CSH Prep C18 OBD Column, 19×250 mm, 5 μm; Mobile Phase A: Water (0.1% FA), Mobile Phase B: ACN; Flow rate: 25 mL/min; Gradient: 10% B to 23% B in 12 min; Wave Length: 254 nm) to afford (E)-1-(4-((4-([1,2,4]triazolo[1,5-a]pyridin-7-yloxy)-3-methylphenyl)amino)pyrido[3,4-d]pyrimidin-6-yl)-3-(2-(azetidin-1-yl)ethylidene)pyrrolidin-2-one (Compound 102) (25.4 mg, 5%) as a white solid. LCMS (ESI, m/z): [M+H]⁺=534.3. ¹H NMR (400 MHz, DMSO-d₆): δ 10.32 (s, 1H), 9.20 (s, 1H), 9.09 (s, 1H), 8.94 (d, J=7.6 Hz, 1H), 8.65 (s, 1H), 8.39 (s, 1H), 7.86-7.82 (m, 2H), 7.23 (d, J=8.8 Hz, 1H), 7.05-7.03 (m, 1H), 6.83 (d, J=2.4 Hz, 1H), 6.41-6.37 (m, 1H), 4.18-4.15 (m, 2H), 3.23-3.17 (m, 6H), 2.90-2.86 (m, 2H), 2.22 (s, 3H), 2.04-1.97 (m, 2H).

### Example S108: Synthesis of (1R,4E,5R)-4 [2-(dimethylamino)ethylidene]-2-{4-[(3-methyl-4-{[1,2,4]triazolo[1,5-a]pyridin-7-yloxy}phenyl)amino]pyrido[3,2-d]pyrimidin-6-yl}-2-azabicyclo[3.1.0]hexan-3-one and (1S,4E,55)-4-[2-(dimethylamino)ethylidene]-2-{4-[(3-methyl-4-{[1,2,4]triazolo[1,5-a]pyridin-7-yloxy}phenyl)amino]pyrido[3,2-d]pyrimidin-6-yl}-2-azabicyclo[3.1.0]hexan-3-one (Compound 103 and Compound 104)

### Step 1. Synthesis of Tert-butyl 4-{2-[(tert-butyldimethylsilyl)oxy]-1-hydroxyethyl}-3-oxo-2-azabicyclo[3.1.0]hexane-2-carboxylate

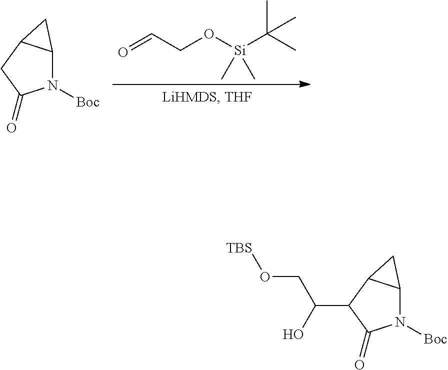

To a solution of tert-butyl 3-oxo-2-azabicyclo[3.1.0]hexane-2-carboxylate (5.0 g, 25.35 mmol) in THF (100.0 mL) was added LiHMDS (51.0 mL, 1 mol/L) at −78° C. under N₂. The mixture was stirred at −78° C. for 2 h under N₂. Then a solution of 2-[(tert-butyldimethylsilyl)oxy]acetaldehyde (5.3 g, 30.42 mmol) in THF (5.0 mL) was added dropwise to the mixture at −78° C. The mixture was stirred at −78° C. for additional 2 h under N₂. After the reaction was completed, the reaction mixture was quenched with NH₄Cl (sat.) solution and extracted with ethyl acetate. The combined organic layer was washed with brine, dried over anhydrous sodium sulfate and filtered. The filtrate was concentrated under vacuum The residue was purified by flash column chromatography with petroleum ether/ethyl acetate (3/1, v/v) to afford tert-butyl 4-{2-[(tert-butyldimethylsilyl)oxy]-1-hydroxyethyl}-3-oxo-2-azabicyclo[3.1.0]hexane-2-carboxylate (3.3 g, crude) as a colorless oil. LCMS (ESI, m/z): [M+H]⁺=372.2.

### Step 2. Synthesis of Tert-butyl-4-{2-[(tert-butyldimethylsilyl)oxy]-1-(methanesulfonyloxy)ethyl}-3-oxo-2-azabicyclo[3.1.0]hexane-2-carboxylate

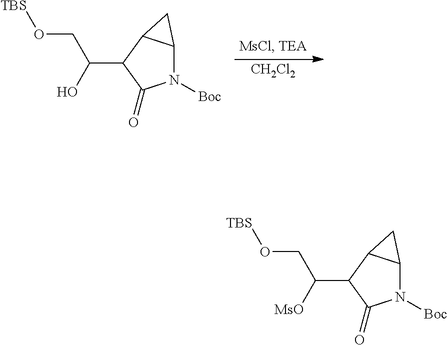

To a solution of tert-butyl-4-{2-[(tert-butyldimethylsilyl)oxy]-1-hydroxyethyl}-3-oxo-2-azabicyclo[3.1.0]hexane-2-carboxylate (3.3 g, crude) in CH₂Cl₂ (50.0 mL) was added TEA (1.8 g, 17.76 mmol) and MsCl (1.7 g, 15.10 mmol) at 0° C. The resulting mixture was stirred at room temperature for 16 h. After the reaction was completed, the mixture was diluted with H₂O and extracted with CH₂Cl₂. The combined organic layer was washed with brine, dried over anhydrous sodium sulfate and filtered. The filtrate was concentrated under vacuum to afford tert-butyl-4-{2-[(tert-butyldimethylsilyl)oxy]-1-(methanesulfonyloxy)ethyl}-3-oxo-2-azabicyclo[3.1.0]hexane-2-carboxylate (3.3 g, crude) as a yellow oil. LCMS (ESI, m/z): [M+H]⁺=450.2.

### Step 3. Synthesis of Tert-butyl-(4E)-4-{2-[(tert-butyldimethylsilyl)oxy]ethylidene}-3-oxo-2-azabicyclo[3.1.0]hexane-2-carboxylate and Tert-butyl-(4Z)-4-{2-[(tert-butyldimethylsilyl)oxy]ethylidene}-3-oxo-2-azabicyclo[3.1.0]hexane-2-carboxylate

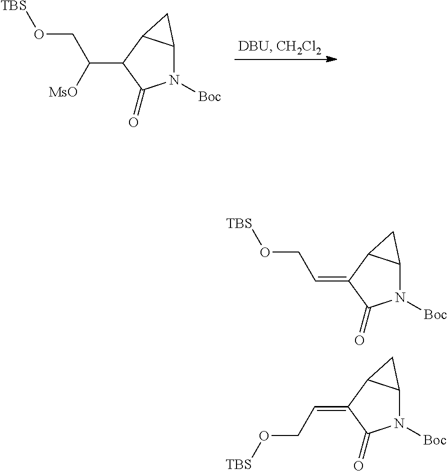

To a solution of tert-butyl-4-{2-[(tert-butyldimethylsilyl)oxy]-1-(methanesulfonyloxy)ethyl}-3-oxo-2-azabicyclo[3.1.0]hexane-2-carboxylate (3.3 g, crude) in CH₂Cl₂ (20.0 mL) was added DBU (2.8 g, 18.34 mmol) at 0° C. under N₂. The resulting mixture was stirred at room temperature for 2 h. After the reaction was completed, the mixture was diluted with H₂O and extracted with CH₂Cl₂. The combined organic layer was washed with brine, dried over anhydrous sodium sulfate and filtered. The filtrate was concentrated under vacuum. The residue was purified by flash column chromatography with petroleum ether/ethyl acetate (5/1, v/v) to afford tert-butyl-(4E)-4-{2-[(tert-butyldimethylsilyl)oxy]ethylidene}-3-oxo-2-azabicyclo[3.1.0]hexane-2-carboxylate (1.5 g, 57%) as a colorless oil and Tert-butyl-(4Z)-4-{2-[(tert-butyldimethylsilyl)oxy]ethylidene}-3-oxo-2-azabicyclo[3.1.0]hexane-2-carboxylate (300.0 mg, 11%) as a colorless oil. LCMS (ESI, m/z): [M+H]⁺=354.2.

### Step 4. Synthesis of Tert-butyl-(4E)-4-(2-hydroxyethylidene)-3-oxo-2-azabicyclo[3.1.0]hexane-2-carboxylate

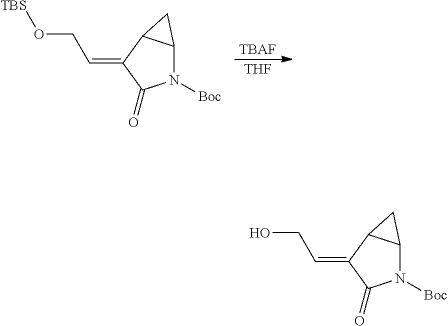

To a mixture of tert-butyl-(4E)-4-{2-[(tert-butyldimethylsilyl)oxy]ethylidene}-3-oxo-2-azabicyclo[3.1.0]hexane-2-carboxylate (1.5 g, 4.24 mmol) in THE (20.0 mL) was added TBAF (3.0 mL, 1 mol/L) at room temperature. The resulting mixture was stirred at room temperature for 2 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford tert-butyl-(4E)-4-(2-hydroxyethylidene)-3-oxo-2-azabicyclo[3.1.0]hexane-2-carboxylate (460.0 mg, 45%) as a light yellow oil. LCMS (ESI, m/z): [M+H]⁺=240.1.

### Step 5. Synthesis of Tert-butyl-(4E)-4-[2-(methanesulfonyloxy)ethylidene]-3-oxo-2-azabicyclo[3.1.0]hexane-2-carboxylate

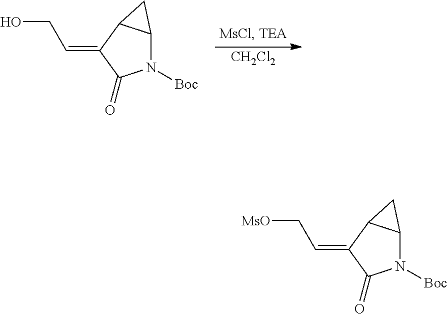

To a solution of tert-butyl-(4E)-4-(2-hydroxyethylidene)-3-oxo-2-azabicyclo[3.1.0]hexane-2-carboxylate (400.0 mg, 1.67 mmol) in CH₂Cl₂ (10.0 mL) was added TEA (422.9 mg, 4.18 mmol) and MsCl (325.5 mg, 2.84 mmol) at 0° C. The resulting mixture was stirred at room temperature for 16 h. After the reaction was completed, the mixture was diluted with H₂O and extracted with CH₂Cl₂. The combined organic layer was washed with brine, dried over anhydrous sodium sulfate and filtered. The filtrate was concentrated under vacuum to afford tert-butyl-(4E)-4-[2-(methanesulfonyloxy)ethylidene]-3-oxo-2-azabicyclo[3.1.0]hexane-2-carboxylate (530.0 mg, crude) as a yellow oil. LCMS (ESI, m/z): [M+H]⁺=318.1.

### Step 6. Synthesis of Tert-butyl-(4E)-4-[2-(dimethylamino)ethylidene]-3-oxo-2-azabicyclo[3.1.0]hexane-2-carboxylate

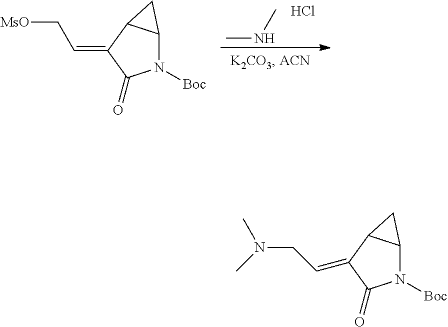

To a solution of tert-butyl-(4E)-4-[2-(methanesulfonyloxy)ethylidene]-3-oxo-2-azabicyclo[3.1.0]hexane-2-carboxylate (530.0 mg, crude) in ACN (15.0 mL) was added dimethylamine hydrochloride (136.1 mg, 1.67 mmol) and K₂CO₃ (1154.0 mg, 8.35 mmol) at room temperature. The resulting mixture was stirred at 60° C. for 2 h. After the reaction was completed, the mixture was diluted with H₂O and extracted with CH₂Cl₂. The combined organic layer was washed with brine, dried over anhydrous sodium sulfate and filtered. The filtrate was concentrated under vacuum. The residue was purified by flash column chromatography with petroleum ether/ethyl acetate (5/1, v/v) to afford tert-butyl-(4E)-4-[2-(dimethylamino)ethylidene]-3-oxo-2-azabicyclo[3.1.0]hexane-2-carboxylate (150.0 mg, 33%) as a yellow oil. LCMS (ESI, m/z): [M+H]⁺=267.2.

### Step 7. Synthesis of (E)-4-(2-(dimethylamino)ethylidene)-2-azabicyclo[3.1.0]hexan-3-one 2,2,2-trifluoroacetate

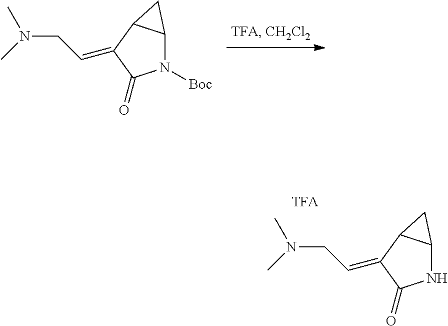 [SMILES: CN(C)C/C=C1/C(=O)N(C(=O)OC(C)(C)C)C2CC12 ; CN(C)C/C=C1/C(=O)NC2CC12 ; "TFA"]

To a solution of tert-butyl-(4E)-4-[2-(dimethylamino)ethylidene]-3-oxo-2-azabicyclo[3.1.0]hexane-2-carboxylate (150.0 mg, 0.56 mmol) in CH₂Cl₂ (5.0 mL) was added TFA (5.0 mL) at 0° C. The resulting mixture was stirred at room temperature for 2 h. After the reaction was completed, the mixture was concentrated under vacuum to afford (E)-4-(2-(dimethylamino)ethylidene)-2-azabicyclo[3.1.0]hexan-3-one 2,2,2-trifluoroacetate (150.0 mg, crude) as a brown oil. LCMS (ESI, m/z): [M+H]⁺=167.1.

### Step 8. Synthesis of (4E)-4-[2-(dimethylamino)ethylidene]-2-{4-[(3-methyl-4-{[1,2,4]triazolo[1,5-a]pyridin-7-yloxy}phenyl)amino]pyrido[3,2-d]pyrimidin-6-yl}-2-azabicyclo[3.1.0]hexan-3-one

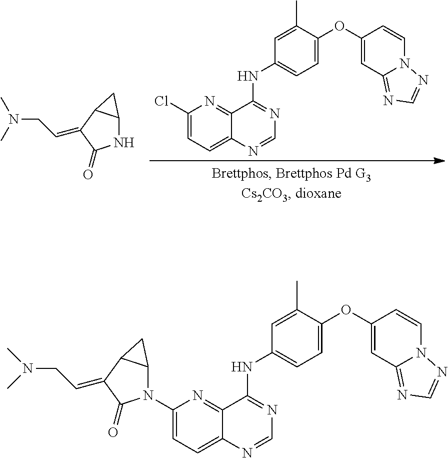 [SMILES: CN(C)C/C=C1/C(=O)NC2CC12 ; Cc1cc(Nc2ncnc3ccc(Cl)nc23)ccc1Oc1ccn2ncnc2c1 ; Cc1cc(Nc2ncnc3ccc(N4C(=O)/C(=C/CN(C)C)C5CC54)nc23)ccc1Oc1ccn2ncnc2c1]

To a solution of (E)-4-(2-(dimethylamino)ethylidene)-2-azabicyclo[3.1.0]hexan-3-one 2,2,2-trifluoroacetate (50.0 mg, crude) in 1,4-dioxane (10.0 mL) were added 6-chloro-N-(3-methyl-4-{[1,2,4]triazolo[1,5-a]pyridin-7-yloxy}phenyl)pyrido[3,2-d]pyrimidin-4-amine (242.9 mg, 0.60 mmol), Cs₂CO₃ (392.0 mg, 1.20 mmol), Pd(OAc)₂ (13.5 mg, 0.06 mmol) and Xphos (57.4 mg, 0.12 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford (4E)-4-[2-(dimethylamino)ethylidene]-2-{4-[(3-methyl-4-{[1,2,4]triazolo[1,5-a]pyridin-7-yloxy}phenyl)amino]pyrido[3,2-d]pyrimidin-6-yl}-2-azabicyclo[3.1.0]hexan-3-one (50.0 mg, 30%) as a yellow solid. LCMS (ESI, m/z): [M+H]⁺=534.2.

### Step 9. Synthesis of (1R,4E,5R)-4-[2-(dimethylamino)ethylidene]-2-{4-[(3-methyl-4-{[1,2,4]triazolo[1,5-a]pyridin-7-yloxy}phenyl)amino]pyrido[3,2-d]pyrimidin-6-yl}-2-azabicyclo[3.1.0]hexan-3-one and (1S,4E,5S)-4-[2-(dimethylamino)ethylidene]-2-{4-1(3-methyl-4-{[1,2,4]triazolo[1,5-a]pyridin-7-yloxy}phenyl)amino]pyrido[3,2-d]pyrimidin-6-yl}-2-azabicyclo[3.1.0]hexan-3-one (Compound 103 and Compound 104)

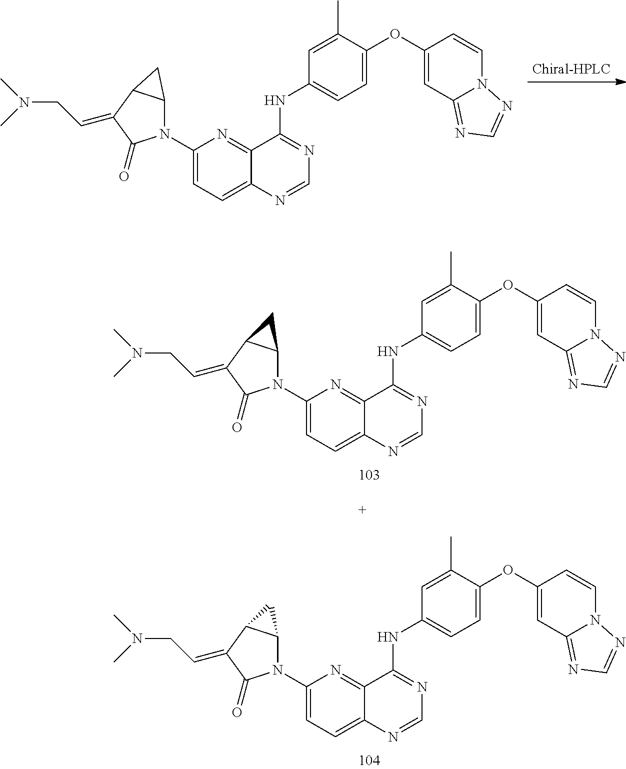 [SMILES: Cc1cc(Nc2ncnc3ccc(N4C(=O)/C(=C/CN(C)C)C5CC54)nc23)ccc1Oc1ccn2ncnc2c1 ; Cc1cc(Nc2ncnc3ccc(N4C(=O)/C(=C/CN(C)C)[C@H]5C[C@H]54)nc23)ccc1Oc1ccn2ncnc2c1 ; Cc1cc(Nc2ncnc3ccc(N4C(=O)/C(=C/CN(C)C)[C@@H]5C[C@@H]54)nc23)ccc1Oc1ccn2ncnc2c1]

The racemic product of (4E)-4-[2-(dimethylamino)ethylidene]-2-{4-[(3-methyl-4-{[1,2,4]triazolo[1,5-a]pyridin-7-yloxy}phenyl)amino]pyrido[3,2-d]pyrimidin-6-yl}-2-azabicyclo[3.1.0]hexan-3-one (50.0 mg, 0.09 mmol) was separated by Prep-Chiral-HPLC with the following conditions: (Column: CHIRAL ART Amylose-SA, 2×25 cm, 5 μm; Mobile Phase A: Hex (0.5% 2M NH₃-MeOH)-HPLC, Mobile Phase B: EtOH: DCM=1: 1-HPLC; Flow rate: 20 mL/min; Gradient: 50% B to 50% B in 7 min; Wave Length: 220/254 nm; RT1(min): 4.81; RT2(min): 6.06) to afford 4-[2-(dimethylamino)ethylidene]-2-{4-[(3-methyl-4-{[1,2,4]triazolo[1,5-a]pyridin-7-yloxy}phenyl)amino]pyrido[3,2-d]pyrimidin-6-yl}-2-azabicyclo[3.1.0]hexan-3-one Enantiomer 1 (RT1: 4.81 min, 13.5 mg, 54%) as a yellow solid and 4-[2-(dimethylamino)ethylidene]-2-{4-[(3-methyl-4-{[1,2,4]triazolo[1,5-a]pyridin-7-yloxy}phenyl)amino]pyrido[3,2-d]pyrimidin-6-yl}-2-azabicyclo[3.1.0]hexan-3-one Enantiomer 2 (RT2: 6.06 min, 15.3 mg, 61%) as a yellow solid. The absolute stereochemistry of Enantiomers 1 and 2 was not assigned. The two enantiomeric structures that could be obtained from chiral separation of the enantiomeric mixture as described above are shown as Compounds 103 and 104 in Table 1.

4-[2-(dimethylamino)ethylidene]-2-{4-[(3-methyl-4-{[1,2,4]triazolo[1,5-a]pyridin-7-yloxy}phenyl)amino]pyrido[3,2-d]pyrimidin-6-yl}-2-azabicyclo[3.1.0]hexan-3-one Enantiomer 1: RT1(min): 4.81; LCMS (ESI, m/z): [M+H]⁺=534.1. ¹H NMR (400 MHz, DMSO-d₆): δ 9.71 (s, 1H), 8.95 (d, J=7.6 Hz, 1H), 8.87 (d, J=9.2 Hz, 1H), 8.64 (s, 1H), 8.39 (s, 1H), 8.27 (d, J=9.2 Hz, 1H), 7.97-7.95 (m, 2H), 7.27 (d, J=9.2 Hz, 1H), 7.05-7.03 (m, 1H), 6.81 (d, J=2.4 Hz, 1H), 6.61-6.57 (m, 1H), 5.12-5.09 (m, 1H), 3.52-3.36 (m, 2H), 2.68-2.58 (m, 1H), 2.33 (s, 6H), 2.23 (s, 3H), 1.47-1.42 (m, 1H), 0.87-0.81 (m, 1H).

4-[2-(dimethylamino)ethylidene]-2-{4-[(3-methyl-4-{[1,2,4]triazolo[1,5-a]pyridin-7-yloxy}phenyl)amino]pyrido[3,2-d]pyrimidin-6-yl}-2-azabicyclo[3.1.0]hexan-3-one Enantiomer 2: RT2(min): 6.06; LCMS (ESI, m/z): [M+H]⁺=534.4. 1H NMR (400 MHz, DMSO-d₆): δ 9.72 (s, 1H), 8.95 (d, J=7.6 Hz, 1H), 8.87 (d, J=9.2 Hz, 1H), 8.64 (s, 1H), 8.39 (s, 1H), 8.28 (d, J=9.2 Hz, 1H), 7.97-7.94 (m, 2H), 7.28-7.26 (m, 1H), 7.06-7.03 (m, 1H), 6.80 (d, J=2.4 Hz, 1H), 6.60-6.57 (m, 1H), 5.14-5.10 (m, 1H), 3.50-3.38 (m, 2H), 2.64-2.59 (m, 1H), 2.35 (s, 6H), 2.23 (s, 3H), 1.47-1.42 (m, 1H), 0.88-0.85 (m, 1H).

### Example S109: Synthesis of (S,E)-1-(4-((4-([1,2,4]triazolo[1,5-a]pyridin-7-yloxy)-3-methylphenyl)amino)pyrido[3,2-d]pyrimidin-6-yl)-3-(2-(dimethylamino)propylidene)pyrrolidin-2-one (Compound 113)

### Step 1. Synthesis of Tert-butyl 3-((25)-2-((tert-butoxycarbonyl)(methyl)amino)-1-hydroxypropyl)-2-oxopyrrolidine-1-carboxylate

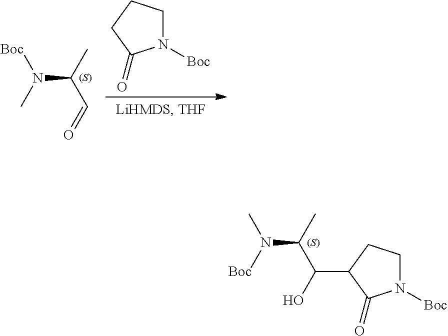

To a solution of tert-butyl 2-oxopyrrolidine-1-carboxylate (3.1 g, 17.09 mmol) in THF (50.0 mL) was added dropwise LiHMDS (50.0 mL, 1 mol/L) at −78° C. under N₂. The mixture was stirred at −78° C. for 1 h. Then a solution of tert-butyl (S)-methyl(1-oxopropan-2-yl)carbamate (5.0 g, 26.70 mmol) in THE (10.0 mL) was added dropwise to the mixture at 78° C. The resulting mixture was stirred at −78° C. for additional 2 h. After the reaction was completed, the mixture was quenched by the addition of sat. NH₄Cl (aq.) and then extracted with ethyl acetate. The combined organic layer was washed with brine, dried over anhydrous sodium sulfate and filtered. The filtrate was concentrated under reduced pressure to afford tert-butyl 3-((2S)-2-((tert-butoxycarbonyl)(methyl)amino)-1-hydroxypropyl)-2-oxopyrrolidine-1-carboxylate (3.0 g, crude) as a yellow oil. LCMS (ESI, m/z): [M+H]⁺=373.2.

### Step 2. Synthesis of Tert-butyl 3-((25)-2-((tert-butoxycarbonyl)(methyl)amino)-1-((methylsulfonyl)oxy)propyl)-2-oxopyrrolidine-1-carboxylate

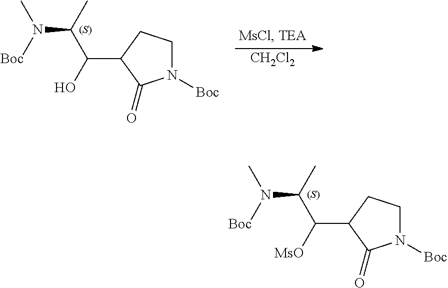

To a solution of tert-butyl 3-((2S)-2-((tert-butoxycarbonyl)(methyl)amino)-1-hydroxypropyl)-2-oxopyrrolidine-1-carboxylate (5.0 g, crude) in DCM (50.0 mL) was added TEA (5.4 g, 53.69 mmol) and MsCl (3.1 g, 26.85 mmol) at 0° C. under N₂. The mixture was stirred at room temperature for 16 h. After the reaction was completed, the mixture was diluted with H₂O and extracted with CH₂Cl₂. The combined organic layer was washed with brine, dried over anhydrous sodium sulfate and filtered. The filtrate was concentrated under reduced pressure to afford tert-butyl 3-((2S)-2-((tert-butoxycarbonyl)(methyl)amino)-1-((methylsulfonyl)oxy)propyl)-2-oxopyrrolidine-1-carboxylate (2.5 g, crude) as a brown oil. LCMS (ESI, m/z): [M+H]⁺=451.2.

### Step 3. Synthesis of Tert-butyl (S,E)-3-(2-((tert-butoxycarbonyl)(methyl)amino)propylidene)-2-oxopyrrolidine-1-carboxylate

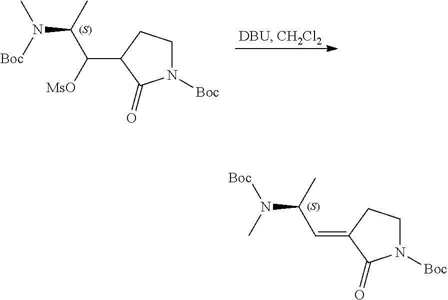

To a solution of tert-butyl 3-((2S)-2-((tert-butoxycarbonyl)(methyl)amino)-1-((methylsulfonyl)oxy)propyl)-2-oxopyrrolidine-1-carboxylate (2.0 g, crude) 1N DCM (20.0 ML) was added DBU (2.7 g, 17.75 mmol) AT ROOM TEMPERATURE. The resulting mixture was stirred at room temperature for 2 h. After the reaction was completed, the mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (45/55, v/v) to afford tert-butyl (S,E)-3-(2-((tert-butoxycarbonyl)(methyl)amino)propylidene)-2-oxopyrrolidine-1-carboxylate (300.0 mg, 19%) as a yellow oil. LCMS (ESI, m/z): [M+H]⁺=355.2.

### Step 4. Synthesis of (S,E)-3-(2-(methylamino)propylidene)pyrrolidin-2-one; 2,2,2-trifluoroacetic acid

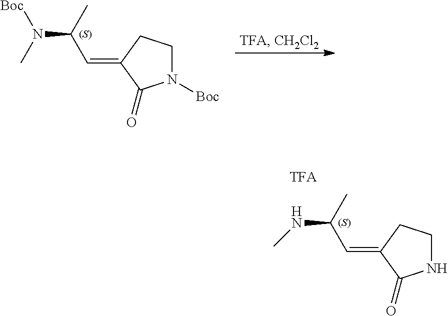

To a solution of tert-butyl (S,E)-3-(2-((tert-butoxycarbonyl)(methyl)amino)propylidene)-2-oxopyrrolidine-1-carboxylate (500.0 mg, 1.41 mmol) in DCM (5.0 mL) was TFA (5.0 mL) AT ROOM TEMPERATURE. The resulting mixture was stirred at room temperature for 2 h. After the reaction was completed, the resulting mixture was concentrated under reduced pressure to afford (S,E)-3-(2-(methylamino)propylidene)pyrrolidin-2-one; 2,2,2-trifluoroacetic acid (300.0 mg, crude) as a yellow oil. LCMS (ESI, m/z): [M+H]⁺=155.1.

### Step 5. Synthesis of (S,E)-3-(2-(dimethylamino)propylidene)pyrrolidin-2-one

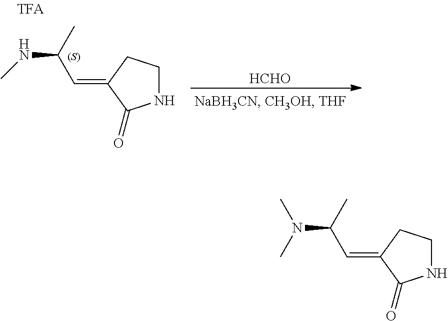

To a solution of (S,E)-3-(2-(methylamino)propylidene)pyrrolidin-2-one; 2,2,2-trifluoroacetic acid (300 mg, crude) in THE (3.0 mL) and MeOH (0.6 mL) was added formaldehyde (175.2 mg, 30%) AT ROOM TEMPERATURE. The resulting mixture was stirred at room temperature for 1 h. Then NaBH₃CN (550.1 mg, 8.75 mmol) was added to the mixture at 0° C. under N₂. The resulting mixture was stirred at room temperature for additional 2 h. After the reaction was completed, the resulting mixture was quenched by H₂O and then concentrated under reduced pressure. The residue was purified by reverse phase flash chromatography with acetonitrile/water (30/70, v/v) to afford (S,E)-3-(2-(dimethylamino)propylidene)pyrrolidin-2-one (200.0 mg, 61%) as a yellow oil. LCMS (ESI, m/z): [M+H]⁺=169.1.

### Step 6. Synthesis of (S,E)-1-(4-((4-([1,2,4]triazolo[1,5-a]pyridin-7-yloxy)-3-methylphenyl)amino)pyrido[3,2-d]pyrimidin-6-yl)-3-(2-(dimethylamino)propylidene)pyrrolidin-2-one (Compound 113)

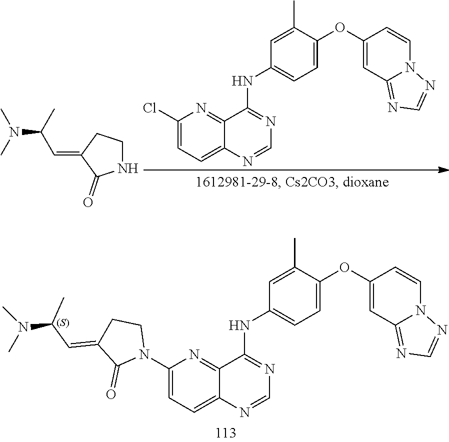

To a solution of (S,E)-3-(2-(dimethylamino)propylidene)pyrrolidin-2-one (100.0 mg, 0.59 mmol) in 1,4-dioxane (5.0 mL) was added Cs₂CO₃ (387.3 mg, 1.18 mmol), Pd-PEPPSI-IPentCl 2-methylpyridine (o-picoline) (50.0 mg, 0.059 mmol) and N-(4-([1,2,4]triazolo[1,5-a]pyridin-7-yloxy)-3-methylphenyl)-6-chloropyrido[3,2-d]pyrimidin-4-amine (240.0 mg, 0.59 mmol) at room temperature under N₂. The final reaction mixture was irradiated with microwave radiation at 120° C. for 2 h under N₂. After the reaction was completed, the mixture was cooled to room temperature and filtered. The filtrate was concentrated under reduced pressure. The residue was purified by flash column chromatography with CH₂Cl₂/MeOH (91/9, v/v) and then purified by Prep-HPLC with the following conditions (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 52% B to 67% B in 8 min; Wave Length: 254 nm) to afford (S,E)-1-(4-((4-([1,2,4]triazolo[1,5-a]pyridin-7-yloxy)-3-methylphenyl)amino)pyrido[3,2-d]pyrimidin-6-yl)-3-(2-(dimethylamino)propylidene)pyrrolidin-2-one (Compound 113) (3.6 mg, 1%) as a yellow solid. LCMS (ESI, m/z): [M+H]⁺=536.3. ¹H NMR (400 MHz, DMSO-d₆): δ 9.61 (s, 1H), 9.00-8.94 (m, 2H), 8.64 (s, 1H), 8.39 (s, 1H), 8.28 (d, J=9.2 Hz, 1H), 8.00-7.97 (m, 2H), 7.26 (d, J=8.4 Hz, 1H), 7.06-7.03 (m, 1H), 6.81 (d, J=2.4 Hz, 1H), 6.54-6.51 (m, 1H), 4.38-4.34 (m, 2H), 3.21-3.17 (m, 1H), 2.92-2.89 (m, 2H), 2.23-2.21 (m, 9H), 1.17 (d, J=6.4 Hz, 3H).

### Example S110: Synthesis of (R,E)-1-(4-((4-([1,2,4]triazolo[1,5-a]pyridin-7-yloxy)-3-methylphenyl)amino)pyrido[3,2-d]pyrimidin-6-yl)-3-(2-(dimethylamino)propylidene)pyrrolidin-2-one (Compound 114)

### Step 1. Synthesis of Tert-butyl 3-((2R)-2-((tert-butoxycarbonyl)(methyl)amino)-1-hydroxypropyl)-2-oxopyrrolidine-1-carboxylate

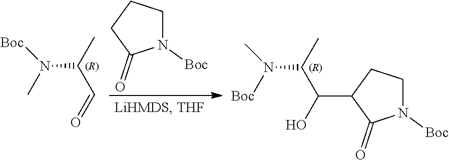

To a solution of tert-butyl 2-oxopyrrolidine-1-carboxylate (3.0 g, 16.22 mmol) in THF (50.0 mL) was added dropwise LiHMDS (50.0 mL, 1.0 mol/L) at −78° C. under N₂. The mixture was stirred at −78° C. for 1 h. Then a solution of tert-butyl (R)-methyl(1-oxopropan-2-yl)carbamate (4.5 g, 24.30 mmol) in THF (10.0 mL) was added dropwise to the mixture at 78° C. The resulting mixture was stirred at −78° C. for additional 2 h. After the reaction was completed, the mixture was quenched by the addition of sat. NH₄Cl (aq.) and then extracted with ethyl acetate. The combined organic layer was washed with brine, dried over anhydrous sodium sulfate and filtered. The filtrate was concentrated under reduced pressure to afford tert-butyl 3-((2R)-2-((tert-butoxycarbonyl)(methyl)amino)-1-hydroxypropyl)-2-oxopyrrolidine-1-carboxylate (2.7 g, crude) as a yellow oil. LCMS (ESI, m/z): [M+H]⁺=373.2.

### Step 2. Synthesis of Tert-butyl 3-((2R)-2-((tert-butoxycarbonyl)(methyl)amino)-1-((methylsulfonyl)oxy)propyl)-2-oxopyrrolidine-1-carboxylate

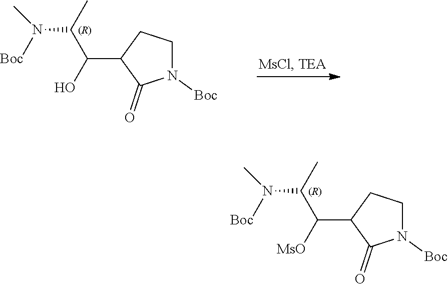

To a solution of tert-butyl 3-((2R)-2-((tert-butoxycarbonyl)(methyl)amino)-1-hydroxypropyl)-2-oxopyrrolidine-1-carboxylate (5.0 g, crude) in DCM (50.0 mL) was added TEA (5.4 g, 53.69 mmol) and MsCl (3.1 g, 26.85 mmol) at 0° C. under N₂. The mixture was stirred at room temperature for 16 h. After the reaction was completed, the mixture was diluted with H₂O and extracted with CH₂Cl₂. The combined organic layer was washed with brine, dried over anhydrous sodium sulfate and filtered. The filtrate was concentrated under reduced pressure to afford tert-butyl 3-((2R)-2-((tert-butoxycarbonyl)(methyl)amino)-1-((methylsulfonyl)oxy)propyl)-2-oxopyrrolidine-1-carboxylate (2.7 g, crude) as a brown oil. LCMS (ESI, m/z): [M+H]⁺=451.2.

### Step 3. Synthesis of Tert-butyl (R,E)-3-(2-((tert-butoxycarbonyl)(methyl)amino)propylidene)-2-oxopyrrolidine-1-carboxylate

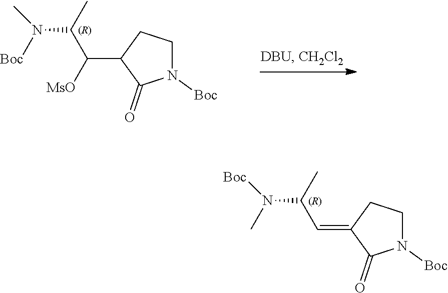

To a solution of tert-butyl 3-((2R)-2-((tert-butoxycarbonyl)(methyl)amino)-1-((methylsulfonyl)oxy)propyl)-2-oxopyrrolidine-1-carboxylate (4.0 g, crude) 1N DCM (50.0 ML) was added DBU (4.1 g, 26.62 mmol) AT ROOM TEMPERATURE. The resulting mixture was stirred at room temperature for 2 h. After the reaction was completed, the mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (57/43, v/v) to afford tert-butyl (R,E)-3-(2-((tert-butoxycarbonyl)(methyl)amino)propylidene)-2-oxopyrrolidine-1-carboxylate (670.0 mg, 21%) as a yellow oil. LCMS (ESI, m/z): [M+H]⁺=355.2.

### Step 4. Synthesis of (R,E)-3-(2-(methylamino)propylidene)pyrrolidin-2-one; 2,2,2-trifluoroacetic acid

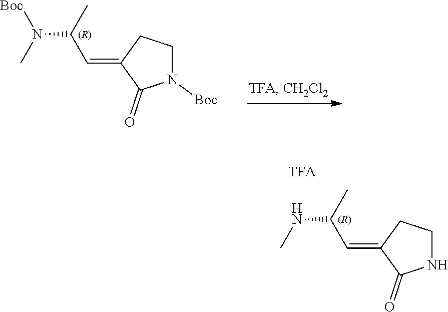

To a solution of tert-butyl (R,E)-3-(2-((tert-butoxycarbonyl)(methyl)amino)propylidene)-2-oxopyrrolidine-1-carboxylate (500.0 mg, 1.41 mmol) in DCM (8.0 mL) was TFA (8.0 mL) AT ROOM TEMPERATURE. The resulting mixture was stirred at room temperature for 2 h. After the reaction was completed, the resulting mixture was concentrated under reduced pressure to afford (R,E)-3-(2-(methylamino)propylidene)pyrrolidin-2-one; 2,2,2-trifluoroacetic acid (200.0 mg, crude) as a yellow oil. LCMS (ESI, m/z): [M+H]⁺=155.1.

### Step 5. Synthesis of (R,E)-3-(2-(dimethylamino)propylidene)pyrrolidin-2-one

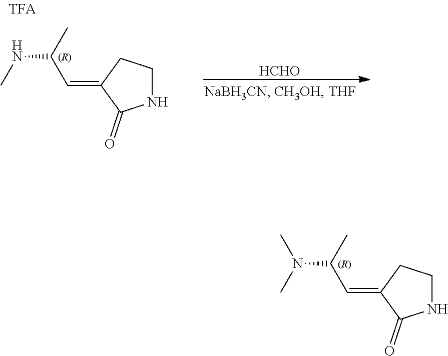

To a solution of (R,E)-3-(2-(methylamino)propylidene)pyrrolidin-2-one; 2,2,2-trifluoroacetic acid (200.0 mg, crude) in THE (3.0 mL) and MeOH (0.6 mL) was added formaldehyde (116.8 mg, 30%) AT ROOM TEMPERATURE. The resulting mixture was stirred at room temperature for 1 h. Then NaBH₃CN (366.7 mg, 5.83 mmol) was added to the mixture at 0° C. under N₂. The resulting mixture was stirred at room temperature for additional 2 h. After the reaction was completed, the resulting mixture was quenched by water and then concentrated under reduced pressure. The residue was purified by reverse phase flash chromatography with acetonitrile/water (45/55, v/v) to afford (R,E)-3-(2-(dimethylamino)propylidene)pyrrolidin-2-one (100.0 mg, 45%) as a yellow oil. LCMS (ESI, m/z): [M+H]⁺=169.1.

### Step 6. Synthesis of (R,E)-1-(4-((4-([1,2,4]triazolo[1,5-a]pyridin-7-yloxy)-3-methylphenyl)amino)pyrido[3,2-d]pyrimidin-6-yl)-3-(2-(dimethylamino)propylidene)pyrrolidin-2-one (Compound 114)

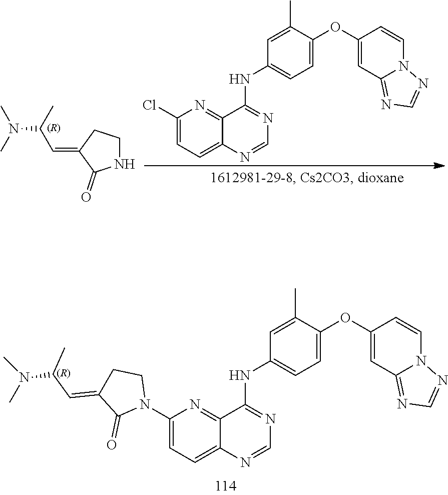

To a solution of (R,E)-3-(2-(dimethylamino)propylidene)pyrrolidin-2-one (100.0 mg, 0.59 mmol) in 1,4-dioxane (8.0 mL) was added Cs₂CO₃ (387.3 mg, 1.18 mmol), Pd-PEPPSI-IPentCl 2-methylpyridine (o-picoline) (50.0 mg, 0.059 mmol) and N-(4-([1,2,4]triazolo[1,5-a]pyridin-7-yloxy)-3-methylphenyl)-6-chloropyrido[3,2-d]pyrimidin-4-amine (240.0 mg, 0.59 mmol) at room temperature under N₂. The final reaction mixture was irradiated with microwave radiation at 120° C. for 2 h. After the reaction was completed, the mixture was cooled to room temperature and filtered. The filtrate was concentrated under reduced pressure. The residue was purified by flash column chromatography with CH₂Cl₂/MeOH (92/8, v/v) and then purified by Prep-HPLC with the following conditions (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 32% B to 48% B in 8 min; Wave Length: 254 nm) to afford (R,E)-1-(4-((4-([1,2,4]triazolo[1,5-a]pyridin-7-yloxy)-3-methylphenyl)amino)pyrido[3,2-d]pyrimidin-6-yl)-3-(2-(dimethylamino)propylidene)pyrrolidin-2-one (Compound 114) (4.9 mg, 2%) as a white solid. LCMS (ESI, m/z): [M+H]⁺=536.2. ¹H NMR (400 MHz, DMSO-d₆): δ 9.60 (s, 1H), 9.00-8.93 (m, 2H), 8.64 (s, 1H), 8.38 (s, 1H), 8.27 (d, J=9.2 Hz, 1H), 8.03-7.91 (m, 2H), 7.26 (d, J=8.0 Hz, 1H), 7.05-7.03 (m, 1H), 6.81 (s, 1H), 6.54-6.51 (m, 1H), 4.39-4.33 (m, 2H), 3.22-3.17 (m, 1H), 2.96-2.88 (m, 2H), 2.25-2.14 (m, 9H), 1.17 (d, J=6.0 Hz, 3H).

### Example S111: Synthesis of S)-1-(4-(4-([1,2,4]triazolo[1,5-a]pyridin-7-yloxy)-3-methylphenylamino)quinazolin-6-yl)-4-((2-(dimethylamino)ethoxy)methyl)-3-methylenepyrrolidin-2-one and (R)-1-(4-(4-([1,2,4]triazolo[1,5-a]pyridin-7-yloxy)-3-methylphenylamino)quinazolin-6-yl)-4-((2-(dimethylamino)ethoxy)methyl)-3-methylenepyrrolidin-2-one (Compound 57 and Compound 58)

### Step 1. Synthesis of Methyl 1-[(4-methoxyphenyl)methyl]-5-oxopyrrolidine-3-carboxylate

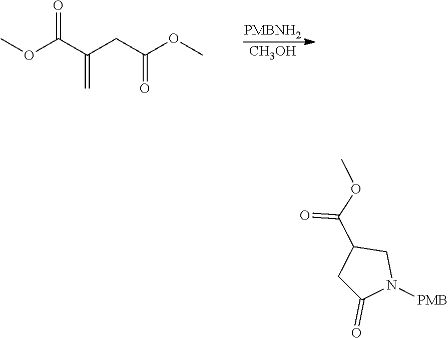

To a solution of 1,4-dimethyl 2-methylidenebutanedioate (20.0 g, 126.46 mmol) in MeOH (200.0 mL) was added (4-methoxyphenyl)methanamine (20.8 g, 151.75 mmol) at room temperature. The resulting mixture was stirred at 80° C. for 16 h. After the reaction was completed. The resulting mixture was concentrated under reduced pressure. The residue was purified by flash chromatography with petroleum ether/ethyl acetate (50/50, v/v) to afforded methyl 1-[(4-methoxyphenyl)methyl]-5-oxopyrrolidine-3-carboxylate (30.0 g, 90%) as a colorless oil. LCMS (ESI, m/z): [M+H]⁺=264.1.

### Step 2. Synthesis of 4-(hydroxymethyl)-1-[(4-methoxyphenyl)methyl]pyrrolidin-2-one

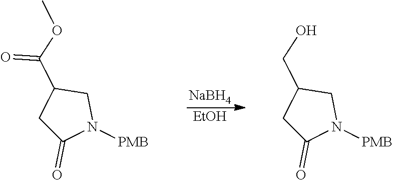

To a solution of methyl 1-[(4-methoxyphenyl)methyl]-5-oxopyrrolidine-3-carboxylate (30.0 g, 113.94 mmol) in ethanol (200.0 mL) was added NaBH₄ (12.9 g, 341.82 mmol) at 0° C. The resulting mixture was stirred at room temperature for 16 h. After the reaction was completed, the resulting mixture was quenched with H₂O and extracted with ethyl acetate. The combined organic layer was washed with brine, dried over anhydrous sodium sulfate and filtered. The filtrate was concentrated under reduced pressure. The residue was purified by flash column chromatography with DCM/MeOH (90/10, v/v) to afford 4-(hydroxymethyl)-1-[(4-methoxyphenyl)methyl]pyrrolidin-2-one (23.0 g, 85%) as a colorless oil. LCMS (ESI, m/z): [M+H]⁺=236.1.

### Step 3. Synthesis of 1-[(4-methoxyphenyl)methyl]-4-{[2-(oxan-2-yloxy)ethoxy]methyl}pyrrolidin-2-one

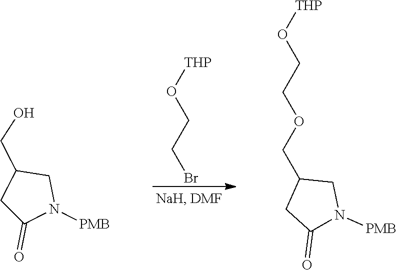

To a solution of 4-(hydroxymethyl)-1-[(4-methoxyphenyl)methyl]pyrrolidin-2-one (15.0 g, 63.75 mmol,) in DMF (200.0 mL) was added NaH (10.2 g, 60%) at room temperature N₂. The resulting mixture was stirred at 60° C. for 1 h and then cooled to room temperature. Then 2-(2-bromoethoxy)oxane (20.0 g, 95.63 mmol) was added to the mixture at room temperature. The resulting mixture was stirred at room temperature for 4 h. After the reaction was completed, the resulting mixture was quenched with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (60/40, v/v) to afford 1-[(4-methoxyphenyl)methyl]-4-{[2-(oxan-2-yloxy)ethoxy]methyl}pyrrolidin-2-one (6.0 g, 25%) as a colorless oil. LCMS (ESI, m/z): [M+H]⁺=364.2.

### Step 4. Synthesis of 1-[(4-methoxyphenyl)methyl]-3-methylidene-4-{[2-(oxan-2-yloxy)ethoxy]methyl}pyrrolidin-2-one

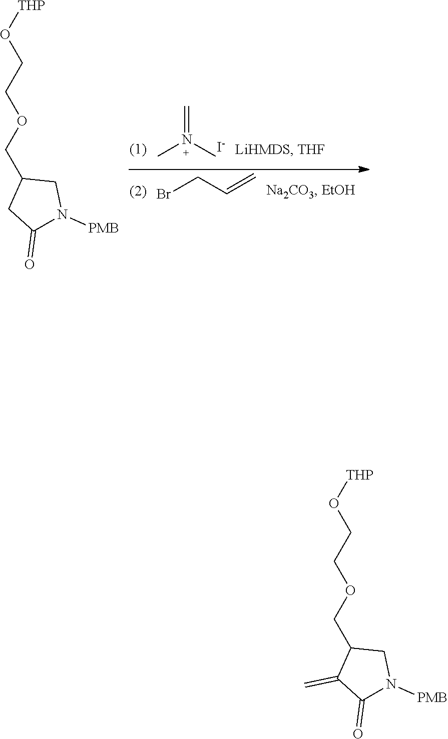

To a solution of 1-[(4-methoxyphenyl)methyl]-4-{[2-(oxan-2-yloxy)ethoxy]methyl}pyrrolidin-2-one (2.7 g, 7.43 mmol) in THE (30.0 mL) was added LiHMDS (14.86 mL, 1 mol/L) at −78° C. under N₂. The mixture was stirred at −78° C. for 50 mm.

Then N-methyl-N-methylenemethanaminium iodide (2.1 g, 11.14 mmol) was added to the mixture. The mixture was stirred at −78° C. for 1.5 h under N₂. The mixture was quenched with saturated NH₄Cl (aq.) and extracted with EtOAc. The combined organic layer was washed with brine, dried over anhydrous sodium sulfate and filtered. The filtrate was concentrated under reduced pressure. The residue was dissolved in EtOH (40.0 mL). Allyl bromide (7.2 g, 59.43 mmol) and Na₂CO₃ (4.7 g, 44.57 mmol) were added to the mixture at room temperature. The resulting mixture was stirred at room temperature for 2 days. After the reaction was completed, the mixture was concentrated under reduced pressure. The residue was purified by flash chromatography with petroleum ether/ethyl acetate (50/30, v/v) to afforded 1-[(4-methoxyphenyl)methyl]-3-methylidene-4-{[2-(oxan-2-yloxy)ethoxy]methyl}pyrrolidin-2-one (200.0 mg, 7%) as a colorless oil. LCMS (ESI, m/z): [M+H]⁺=376.2.

### Step 5. Synthesis of 4-[(2-hydroxyethoxy)methyl]-1-[(4-methoxyphenyl)methyl]-3-methylidenepyrrolidin-2-one

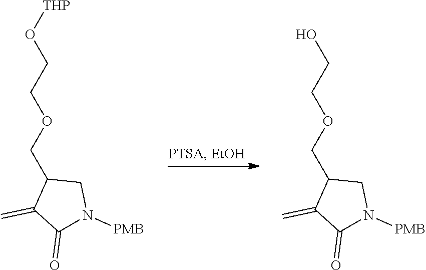

To a solution of 1-[(4-methoxyphenyl)methyl]-3-methylidene-4-{[2-(oxan-2-yloxy)ethoxy]methyl}pyrrolidin-2-one (500.0 mg, 1.33 mmol) in EtOH (10.0 mL) was added PTSA (45.8 mg, 0.27 mmol) at room temperature The resulting mixture was stirred at 80° C. for 16 h. After the reaction was completed, the mixture was concentrated under reduced pressure. The residue was purified by flash column chromatography with CH₂Cl₂/MeOH (90/10, v/v) to afford 4-[(2-hydroxyethoxy)methyl]-1-[(4-methoxyphenyl)methyl]-3-methylidenepyrrolidin-2-one (200.0 mg, 53%) as a yellow oil. LCMS (ESI, m/z): [M+H]⁺=292.1.

### Step 6. Synthesis of 2-({1-[(4-methoxyphenyl)methyl]-4-methylidene-5-oxopyrrolidin-3-yl}methoxy)ethyl 4-methylbenzenesulfonate

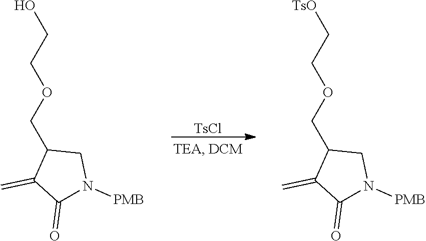

To a mixture of 4-[(2-hydroxyethoxy)methyl]-1-[(4-methoxyphenyl)methyl]-3-methylidenepyrrolidin-2-one (600.0 mg, 2.06 mmol) and TEA (833.6 mg, 8.24 mmol) in DCM (10.0 mL) was added TsCl (1.2 g, 6.2 mmol) at room temperature. The resulting mixture was stirred at room temperature for 4 h. After the reaction was completed, the resulting mixture was quenched with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (90/10, v/v) to afford 2-({1-[(4-methoxyphenyl)methyl]-4-methylidene-5-oxopyrrolidin-3-yl}methoxy)ethyl 4-methylbenzenesulfonate (350.0 mg, 38%) as a yellow oil. LCMS (ESI, m/z): [M+H]⁺=446.2.

### Step 7. Synthesis of 4-((2-(dimethylamino)ethoxy)methyl)-1-(4-methoxybenzyl)-3-methylenepyrrolidin-2-one

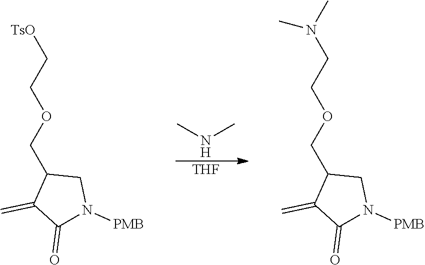

To a solution of 2-({1-[(4-methoxyphenyl)methyl]-4-methylidene-5-oxopyrrolidin-3-yl}methoxy)ethyl 4-methylbenzenesulfonate (445.2 mg, 1.0 mmol) in THF (10.0 mL) was added a solution of dimethylamine in THF (5.0 mL, 2.0 mol/L) at room temperature. The resulting mixture was stirred at 60° C. for 16 h. After the reaction was completed, the mixture was concentrated under reduced pressure. The residue was purified by flash column chromatography with CH₂Cl₂/MeOH (90/10, v/v) to afford 4-((2-(dimethylamino)ethoxy)methyl)-1-(4-methoxybenzyl)-3-methylenepyrrolidin-2-one (200.0 mg, 44%) as a yellow oil. LCMS (ESI, m/z): [M+H]⁺=319.2.

### Step 8. Synthesis of 4-((2-(dimethylamino)ethoxy)methyl)-3-methylenepyrrolidin-2-one

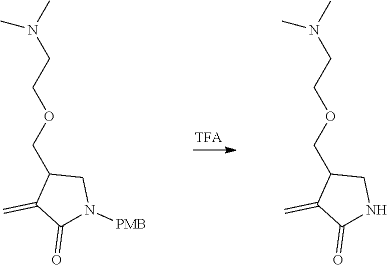

The solution of 4-((2-(dimethylamino)ethoxy)methyl)-1-(4-methoxybenzyl)-3-methylenepyrrolidin-2-one (319.0 mg, 0.99 mmol) in TFA (10.0 mL) stirred at 100° C. for 16 h. After the reaction was completed, the mixture was concentrated under reduced pressure. The residue was purified by reverse phase flash column chromatography with CH₃CN/H₂O (10/90, v/v) to afford 4-((2-(dimethylamino)ethoxy)methyl)-3-methylenepyrrolidin-2-one (100.0 mg, 31%) as a yellow oil. LCMS (ESI, m/z): [M+H]⁺=199.1.

### Step 9. Synthesis of 1-(4-(4-([1,2,4]triazolo[1,5-a]pyridin-7-yloxy)-3-methylphenylamino)quinazolin-6-yl)-4-((2-(dimethylamino)ethoxy)methyl)-3-methylenepyrrolidin-2-one

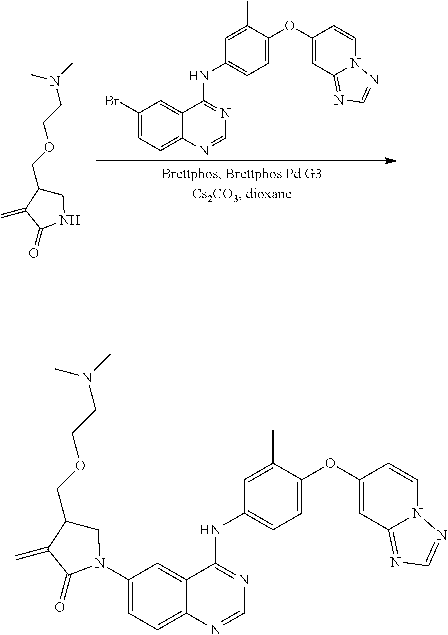

To a solution of 4-((2-(dimethylamino)ethoxy)methyl)-3-methylenepyrrolidin-2-one (99.0 mg, 0.50 mmol) in 1,4-dioxane (10.0 mL) was added N-(4-([1,2,4]triazolo[1,5-a]pyridin-7-yloxy)-3-methylphenyl)-6-bromoquinazolin-4-amine (223.0 mg, 0.50 mmol), BrettPhos (53.6 mg, 0.10 mmol), Cs₂CO₃ (489.0 mg, 1.5 mmol) and BrettPhos Pd G3 (45.3 mg, 0.05 mmol) at room temperature under N₂. The resulting mixture was stirred at 100° C. for 16 h. After the reaction was completed, the resulting mixture was filtered. The filtrate was concentrated under reduced pressure. The residue was purified by flash column chromatography with petroleum DCM/MeOH (5/1, v/v) to afford 1-(4-(4-([1,2,4]triazolo[1,5-a]pyridin-7-yloxy)-3-methylphenylamino)quinazolin-6-yl)-4-((2-(dimethylamino)ethoxy)methyl)-3-methylenepyrrolidin-2-one (60.0 mg, 21%) as a yellow solid. LCMS (ESI, m/z): [M+H]⁺=565.3.

### Step 10. Synthesis of (S)-1-(4-(4-([1,2,4]triazolo[1,5-a]pyridin-7-yloxy)-3-methylphenylamino)quinazolin-6-yl)-4-((2-(dimethylamino)ethoxy)methyl)-3-methylenepyrrolidin-2-one and (R)-1-(4-(4-([1,2,4]triazolo[1,5-a]pyridin-7-yloxy)-3-methylphenylamino)quinazolin-6-yl)-4-((2-(dimethylamino)ethoxy)methyl)-3-methylenepyrrolidin-2-one (Compound 57 and Compound 58)

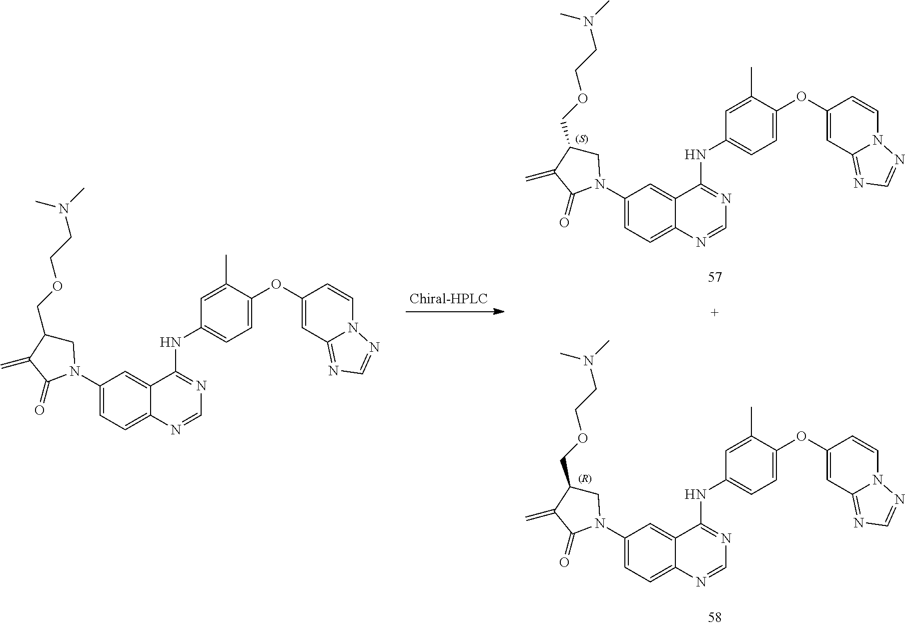

The product of 1-(4-(4-([1,2,4]triazolo[1,5-a]pyridin-7-yloxy)-3-methylphenylamino)quinazolin-6-yl)-4-((2-(dimethylamino)ethoxy)methyl)-3-methylenepyrrolidin-2-one (60.0 mg, 0.11 mmol) was separated by Prep-Chiral-HPLC with the following conditions (Column: CHIRAL ART Amylose-SA, 2×25 cm, 5 μm; Mobile Phase A: Hex (0.5% 2M NH₃-MeOH)-HPLC, Mobile Phase B: EtOH: DCM=1: 1-HPLC; Flow rate: 20 mL/min; Gradient: 30% B to 30% B in 13 min; Wave Length: 220/254 nm; RT1(min): 8.32; RT2(min): 10.64) to afford (S)-1-(4-(4-([1,2,4]triazolo[1,5-a]pyridin-7-yloxy)-3-methylphenylamino)quinazolin-6-yl)-4-((2-(dimethylamino)ethoxy)methyl)-3-methylenepyrrolidin-2-one Enantiomer 1 (RT1 8.32 min, 8.5 mg, 28%) as a white solid and (R) 1-(4-(4-([1,2,4]triazolo[1,5-a]pyridin-7-yloxy)-3-methylphenylamino)quinazolin-6-yl)-4-((2-(dimethylamino)ethoxy)methyl)-3-methylenepyrrolidin-2-one Enantiomer 2 (RT2 10.64 min; 9.0 mg, 30%) as a white solid. The absolute stereochemistry of Enantiomers 1 and 2 was not assigned. The two enantiomeric structures that could be obtained from chiral separation of the enantiomeric mixture as described above are shown as Compounds 57 and 58 in Table 1.

1-(4-(4-([1,2,4]triazolo[1,5-a]pyridin-7-yloxy)-3-methylphenylamino)quinazolin-6-yl)-4-((2-(dimethylamino)ethoxy)methyl)-3-methylenepyrrolidin-2-one Enantiomer 1: RT1(min): 8.32; LCMS (ESI, m/z): [M+H]⁺=565.3. 1H NMR (400 MHz, DMSO-d₆): δ 9.91 (s, 1H), 8.95 (d, J=7.2 Hz, 1H), 8.75 (d, J=8.4 Hz, 1H), 8.59 (s, 1H), 8.39-8.33 (m, 2H), 7.86-7.81 (m, 3H), 7.24 (d, J=8.8 Hz, 1H), 7.05-7.03 (m, 1H), 6.82 (s, 1H), 6.07 (s, 1H), 5.67 (s, 1H), 4.26-4.18 (m, 1H), 3.87-3.84 (m, 1H), 3.78-3.70 (m, 1H), 3.60-3.52 (m, 3H), 3.43-3.39 (m, 1H), 2.67-2.60 (m, 1H), 2.42-2.06 (m, 10H).

1-(4-(4-([1,2,4]triazolo[1,5-a]pyridin-7-yloxy)-3-methylphenylamino)quinazolin-6-yl)-4-((2-(dimethylamino)ethoxy)methyl)-3-methylenepyrrolidin-2-one Enantiomer 2: RT2(min): 10.64; LCMS (ESI, m/z): [M+H]⁺=565.3. 1H NMR (400 MHz, DMSO-d₆): δ 9.91 (s, 1H), 8.95 (d, J=7.6 Hz, 1H), 8.76 (d, J=9.2 Hz, 1H), 8.59 (s, 1H), 8.39-8.33 (m, 2H), 7.86-7.81 (m, 3H), 7.24 (d, J=9.2 Hz, 1H), 7.06-7.04 (m, 1H), 6.83 (s, 1H), 6.07 (s, 1H), 5.67 (s, 1H), 4.22-4.17 (m, 1H), 3.87-3.84 (m, 1H), 3.70-3.62 m, 1H), 3.59-3.52 (m, 3H), 3.42-3.38 (m, 1H), 2.46-2.43 (m, 2H), 2.22 (s, 3H), 2.16 (s, 6H).

### Example S112: Synthesis of (3E)-3 [2-(dimethylamino)ethylidene]-1-[4-({3-fluoro-4-[(1-methyl-1,3-benzodiazol-5 yl)oxy]phenyl}amino)pyrido[3,4-d]pyrimidin-6-yl]pyrrolidin-2-one (Compound 128)

### Step 1. Synthesis of 5-(2-fluoro-4-nitrophenoxy)-1-methyl-1,3-benzodiazole

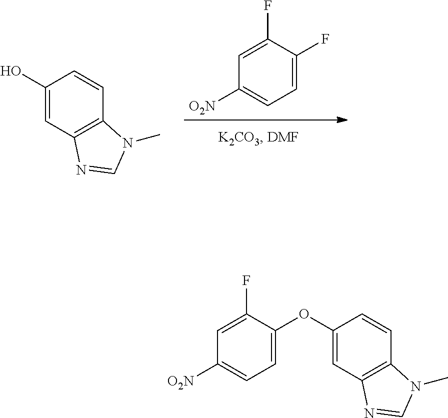 [SMILES: Cn1cnc2cc(O)ccc21 ; O=[N+]([O-])c1ccc(F)c(F)c1 ; Cn1cnc2cc(Oc3ccc([N+](=O)[O-])cc3F)ccc21]

To a solution of 1,2-difluoro-4-nitrobenzene (1.0 g, 6.29 mmol) in DMF (30.0 mL) was added 1-methyl-1,3-benzodiazol-5-ol (0.9 g, 6.29 mmol) and K₂CO₃ (2.6 g, 18.86 mmol) at room temperature. The resulting mixture was stirred at 70° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford 5-(2-fluoro-4-nitrophenoxy)-1-methyl-1,3-benzodiazole (1.2 g, crude) as a yellow solid. LCMS (ESI, m/z): [M+H]⁺=288.1.

### Step 2. Synthesis of 3-fluoro-4-[(1-methyl-1,3-benzodiazol-5-yl)oxy]aniline

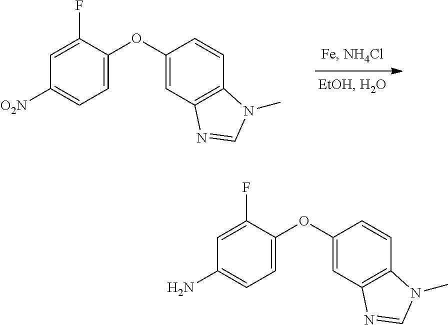 [SMILES: Cn1cnc2cc(Oc3ccc([N+](=O)[O-])cc3F)ccc21 ; Cn1cnc2cc(Oc3ccc(N)cc3F)ccc21]

To a solution of 5-(2-fluoro-4-nitrophenoxy)-1-methyl-1,3-benzodiazole (1.2 g, crude) in EtOH/H₂O (20.0 mL/4.0 mL) was added NH₄Cl (2.1 g, 40.03 mmol) and Fe (2.2 g, 40.03 mmol) at 80° C. The resulting mixture was stirred at 80° C. for 1 h. After the reaction was completed, the resulting mixture was filtered. The filtrate was concentrated under reduced pressure. The residue was purified by flash column chromatography with DCM/MeOH (97/3, v/v) to afford 3-fluoro-4-[(1-methyl-1,3-benzodiazol-5-yl)oxy]aniline (988.0 mg, 95%) as a yellow solid. LCMS (ESI, m/z): [M+H]⁺=258.2.

### Step 3. Synthesis of 6-chloro-N-{3-fluoro-4-[(1-methyl-1,3-benzodiazol-5-yl)oxy]phenyl}pyrido[3,4-d]pyrimidin-4-amine

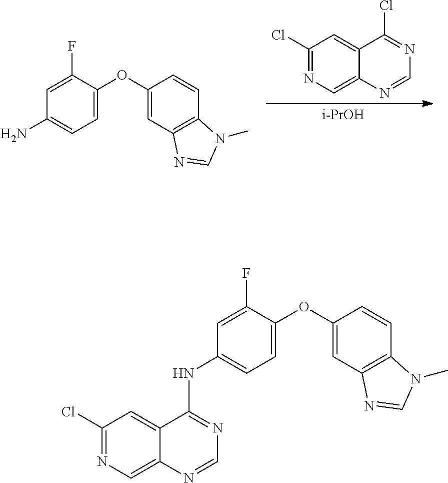 [SMILES: "i-PrOH" ; Cn1cnc2cc(Oc3ccc(N)cc3F)ccc21 ; Clc1cc2c(Cl)ncnc2cn1 ; Cn1cnc2cc(Oc3ccc(Nc4ncnc5cnc(Cl)cc45)cc3F)ccc21]

To a solution of 3-fluoro-4-[(1-methyl-1,3-benzodiazol-5-yl)oxy]aniline (200.0 mg, 0.78 mmol) in i-PrOH (10.0 mL) was added 4,6-dichloropyrido[3,4-d]pyrimidine (155.5 mg, 0.78 mmol) at room temperature. The resulting mixture was stirred at room temperature for 2 h. After the reaction was completed, the resulting mixture was concentrated under reduced pressure. The residue was purified by flash column chromatography with DCM/MeOH (5/1, v/v) to afford 6-chloro-N-{3-fluoro-4-[(1-methyl-1,3-benzodiazol-5-yl)oxy]phenyl}pyrido[3,4-d]pyrimidin-4-amine (288.0 mg, 88%) as a yellow solid. LCMS (ESI, m/z): [M+H]⁺=421.1.

### Step 4. Synthesis of (3E)-3-[2-(dimethylamino)ethylidene]-1-[4-({3-fluoro-4-[(1-methyl-1,3-benzodiazol-5-yl)oxy]phenyl}amino)pyrido[3,4-d]pyrimidin-6-yl]pyrrolidin-2-one (Compound 128)

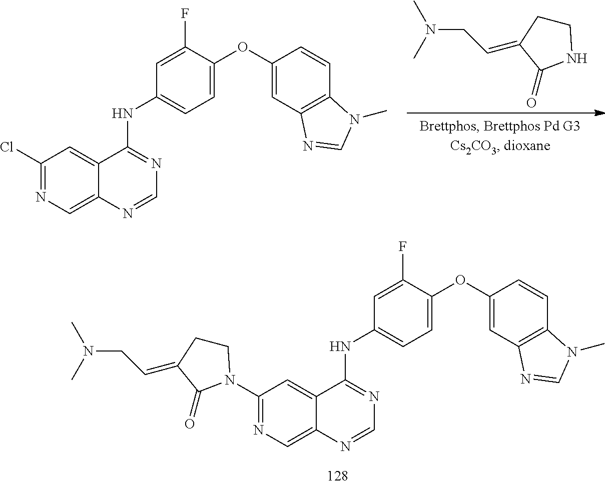

To a solution of 6-chloro-N-{3-fluoro-4-[(1-methyl-1,3-benzodiazol-5-yl)oxy]phenyl}pyrido[3,4-d]pyrimidin-4-amine (100.0 mg, 0.24 mmol) in dioxane (5.0 mL) was added (3E)-3-[2-(dimethylamino)ethylidene]pyrrolidin-2-one (36.6 mg, 0.24 mmol), Cs₂CO₃ (232.3 mg, 0.71 mmol), BrettPhos (25.5 mg, 0.05 mmol) and BrettPhos Pd G3 (21.5 mg, 0.02 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with CH₃CN/H₂O (1/1, v/v) and then purified by Prep-HPLC with the following conditions (Column: Xselect CSH C18 OBD Column 30×150 mm 5 μm; Mobile Phase A: Water (0.1% FA), Mobile Phase B: ACN; Flow rate: 60 mL/min; Gradient: 5% B to 20% B in 10 min; Wave Length: 254 nm) to afford (3E)-3-[2-(dimethylamino)ethylidene]-1-[4-({3-fluoro-4-[(1-methyl-1,3-benzodiazol-5-yl)oxy]phenyl}amino)pyrido[3,4-d]pyrimidin-6-yl]pyrrolidin-2-one (Compound 128) (14.6 mg, 11%) as a yellow solid. LCMS (ESI, m/z): [M+H]⁺=539.2. ¹H NMR (400 MHz, DMSO-d₆): δ 10.39 (s, 1H), 9.20 (s, 1H), 9.10 (s, 1H), 8.67 (s, 1H), 8.21 (s, 1H), 8.06-8.03 (m, 1H), 7.65-7.59 (m, 2H), 7.24 (s, 1H), 7.17-7.07 (m, 2H), 6.56-6.52 (m, 1H), 4.19-4.11 (m, 2H), 3.86 (s, 3H), 3.11 (d, J=6.4 Hz, 2H), 2.95-2.86 (m, 2H), 2.20 (s, 6H).

### Example S113: Synthesis of (3E)-3 [2-(dimethylamino)ethylidene]-1-{4-[(3-fluoro-4-{[1,2,4]triazolo[1,5-a]pyridin-7-yloxy}phenyl)amino]pyrido[3,4-d]pyrimidin-6-yl}pyrrolidin-2-one (Compound 129)

### Step 1. Synthesis of 6-chloro-N-(3-fluoro-4-{[1,2,4]triazolo[1,5-a]pyridin-7-yloxy}phenyl)pyrido[3,4-d]pyrimidin-4-amine

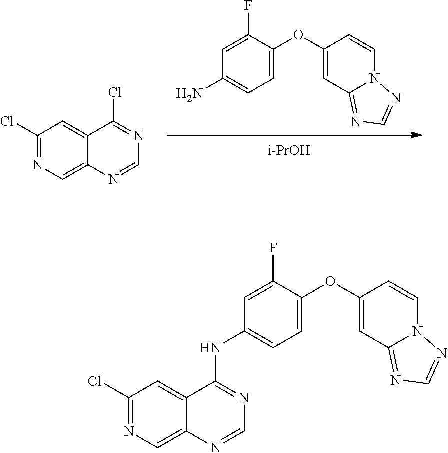 [SMILES: "i-PrOH" ; Clc1cc2c(Cl)ncnc2cn1 ; Nc1ccc(Oc2ccn3ncnc3c2)c(F)c1 ; Fc1cc(Nc2ncnc3cnc(Cl)cc23)ccc1Oc1ccn2ncnc2c1]

To a mixture of 4,6-dichloropyrido[3,4-d]pyrimidine (830.0 mg, crude) in i-PrOH (10.0 mL) was added 3-fluoro-4-{[1,2,4]triazolo[1,5-a]pyridin-7-yloxy}aniline (1010.0 mg, 4.14 mmol) at 0° C. The resulting mixture was stirred at room temperature for 16 h. After the reaction was completed, the mixture was concentrated under reduced pressure. The residue was purified by flash column chromatography with CH₂Cl₂/EtOAc (1/1, v/v) to afford 6-chloro-N-(3-fluoro-4-{[1,2,4]triazolo[1,5-a]pyridin-7-yloxy}phenyl)pyrido[3,4-d]pyrimidin-4-amine (550.0 mg, 32%) as a white solid. LCMS (ESI, m/z): [M+H]⁺=408.1.

### Step 2. Synthesis of (3E)-3-[2-(dimethylamino)ethylidene]-1-{4-[(3-fluoro-4-{[1,2,4]triazolo[1,5-a]pyridin-7-yloxy}phenyl)amino]pyrido[3,4-d]pyrimidin-6-yl}pyrrolidin-2-one (Compound 129)

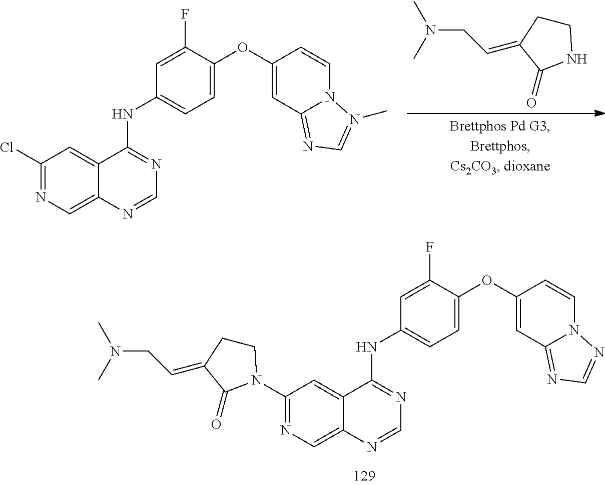

To a mixture of (3E)-3-[2-(dimethylamino)ethylidene]pyrrolidin-2-one (56.7 mg, 0.37 mmol) in dioxane (10.0 mL) was added 6-chloro-N-(3-fluoro-4-{[1,2,4]triazolo[1,5-a]pyridin-7-yloxy}phenyl)pyrido[3,4-d]pyrimidin-4-amine (150.0 mg, 0.37 mmol), Cs₂CO₃ (239.7 mg, 0.74 mmol), BrettPhos (79.0 mg, 0.15 mmol) and BrettPhos Pd G3 (66.7 mg, 0.07 mmol) at room temperature under N₂. The resulting mixture was stirred at 100° C. for 2 h. After the reaction was completed, the resulting mixture was filtered. The filtrate was concentrated under reduced pressure. The residue was purified by flash column chromatography with CH₂Cl₂/MeOH (10/1, v/v) and then purified by Prep-HPLC with the following conditions: (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 27% B to 37% B in 8 min; Wave Length: 254 nm) to afford to afford (3E)-3-[2-(dimethylamino)ethylidene]-1-{4-[(3-fluoro-4-{[1,2,4]triazolo[1,5-a]pyridin-7-yloxy}phenyl)amino]pyrido[3,4-d]pyrimidin-6-yl}pyrrolidin-2-one (Compound 129) (25.2 mg, 13%) as a yellow solid. LCMS (ESI, m/z): [M+H]⁺=526.1. ¹H NMR (400 MHz, DMSO-d₆): δ 10.48 (s, 1H), 9.23 (s, 1H), 9.11 (s, 1H), 8.97 (d, J=7.6 Hz, 1H), 8.71 (s, 1H), 8.43 (s, 1H), 8.18-8.16 (m, 1H), 7.86-7.77 (m, 1H), 7.51-7.47 (m, 1H), 7.12-7.07 (m, 2H), 6.57-6.55 (m, 1H), 4.19-4.16 (m, 2H), 3.10 (d, J=6.0 Hz, 2H), 2.88-2.85 (m, 2H), 2.21 (s, 6H).

### Example S114: Synthesis of (3E)-3-[2-(dimethylamino)ethylidene]-1-[4-({4-[(5 fluoropyridin-3 yl)oxy]-3-methylphenyl}amino)pyrido[3,2-d]pyrimidin-6-yl]pyrrolidin-2-one (Compound 130)

### Step 1. Synthesis of 6-chloro-N-{4-[(5-fluoropyridin-3-yl)oxy]-3-methylphenyl}pyrido[3,2-d]pyrimidin-4-amine

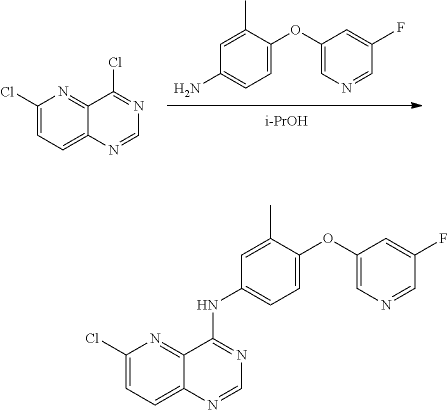 [SMILES: "i-PrOH" ; Clc1ccc2ncnc(Cl)c2n1 ; Cc1cc(N)ccc1Oc1cncc(F)c1 ; Cc1cc(Nc2ncnc3ccc(Cl)nc23)ccc1Oc1cncc(F)c1]

A mixture of 4,6-dichloropyrido[3,2-d]pyrimidine (500.0 mg, crude) and 4-[(5-fluoropyridin-3-yl)oxy]-3-methylaniline (545.5 mg, 2.50 mmol) in i-PrOH (10.0 mL) was stirred at room temperature for 2 h. After the reaction was completed, the resulting mixture was concentrated under vacuum. The residue was purified by flash column chromatography with CH₂Cl₂/CH₃OH (84/16, v/v) to afford 6-chloro-N-{4-[(5-fluoropyridin-3-yl)oxy]-3-methylphenyl}pyrido[3,2-d]pyrimidin-4-amine (800.0 mg, 83%) as a yellow solid. LCMS (ESI, m/z): [M+H]⁺=382.1

### Step 2. Synthesis of (3E)-3-[2-(dimethylamino)ethylidene]-1-[4-({4-[(5-fluoropyridin-3-yl)oxy]-3-methylphenyl}amino)pyrido[3,2-d]pyrimidin-6-yl]pyrrolidin-2-one (Compound 130)

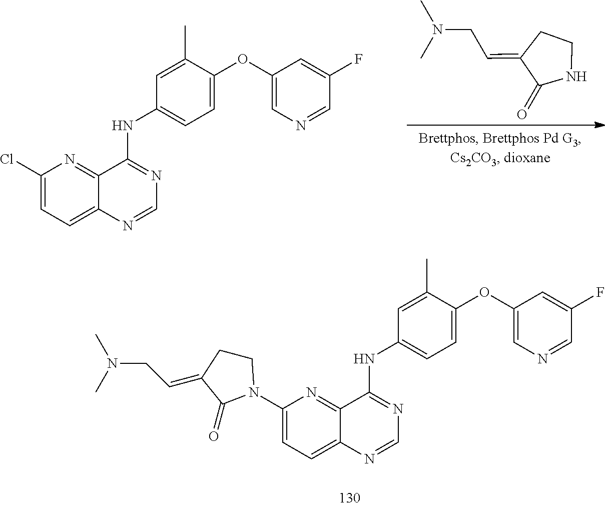 [SMILES: Cc1cc(Nc2ncnc3ccc(Cl)nc23)ccc1Oc1cncc(F)c1 ; CN(C)C/C=C1\CCNC1=O ; Cc1cc(Nc2ncnc3ccc(N4CC/C(=C\CN(C)C)C4=O)nc23)ccc1Oc1cncc(F)c1]

A mixture of 6-chloro-N-{4-[(5-fluoropyridin-3-yl)oxy]-3-methylphenyl}pyrido[3,2-d]pyrimidin-4-amine (200.0 mg, 0.52 mmol), (3E)-3-[2-(dimethylamino)ethylidene]pyrrolidin-2-one (80.8 mg, 0.52 mmol), BrettPhos (56.2 mg, 0.11 mmol), BrettPhos Pd G3 (47.4 mg, 0.05 mmol) and Cs₂CO₃ (512.1 mg, 1.57 mmol) in dioxane (10.0 mL) was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (75/25, v/v) and then purified by Prep-HPLC with the following conditions (Column: Xselect CSH C18 OBD Column 30×150 mm, 5 μm; Mobile Phase A: Water (10 mmol/L NH₄HCO₃), Mobile Phase B: ACN; Flow rate: 60 mL/min; Gradient: 45% B to 51% B in 8 min, 254 nm) to afford (3E)-3-[2-(dimethylamino)ethylidene]-1-[4-({4-[(5-fluoropyridin-3-yl)oxy]-3-methylphenyl}amino)pyrido[3,2-d]pyrimidin-6-yl]pyrrolidin-2-one (Compound 130) (6.4 mg, 2%) as a yellow solid. LCMS (ESI, m/z): [M+H]⁺=500.1. ¹H NMR (400 MHz, DMSO-d₆): δ 9.55 (s, 1H), 8.98 (d, J=8.4 Hz, 1H), 8.63 (s, 1H), 8.35-8.22 (m, 3H), 7.95-7.87 (m, 2H), 7.30 (d, J=8.4 Hz, 1H), 7.16 (d, J=6.4 Hz, 1H), 6.61-6.55 (m, 1H), 4.36-4.31 (m, 2H), 3.17-3.11 (m, 2H), 2.87-2.77 (m, 2H), 2.27-2.21 (m, 9H).

### Example S115: Synthesis of (3E,4R)-3 [2-(dimethylamino)ethylidene]-1-[4-({2-fluoro-5-methyl-4-[(1-methyl-1,2,3-benzotriazol-5 yl)oxy]phenyl}amino)pyrido[3,4-d]pyrimidin-6-yl]-4-methylpyrrolidin-2-one and (3E,4S)-3-[2-(dimethylamino)ethylidene]-1-[4-({2-fluoro-5-methyl-4-[(1-methyl-1,2,3-benzotriazol-5 yl)oxy]phenyl}amino)pyrido[3,4-d]pyrimidin-6-yl]-4-methylpyrrolidin-2-one (Compound 131 and Compound 132)

### Step 1. Synthesis of 5-(5-fluoro-2-methyl-4-nitrophenoxy)-1-methyl-1,2,3-benzotriazole

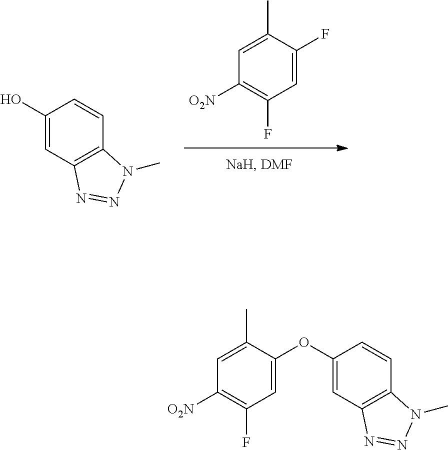 [SMILES: Cn1nnc2cc(O)ccc21 ; Cc1cc([N+](=O)[O-])c(F)cc1F ; Cc1cc([N+](=O)[O-])c(F)cc1Oc1ccc2c(c1)nnn2C]

To a solution of 1-methyl-1,2,3-benzotriazol-5-ol (500.0 mg, 3.35 mmol) in DMF (10.0 mL) was added NaH (120.6 mg, 60%) at 0° C. under N₂. The mixture was stirred at 0° C. for 1 h. Then a solution of 1,5-difluoro-2-methyl-4-nitrobenzene (580.3 mg, 3.35 mmol) in DMF (2.0 mL) was added dropwise to the mixture at 0° C. under N₂. The resulting mixture was stirred at 90° C. for 4 h. After the reaction was completed, the mixture was quenched by the addition of sat. NH₄Cl (aq.). The resulting mixture was extracted with ethyl acetate. The combined organic layer was washed with brine, dried over anhydrous sodium sulfate and filtered. The filtrate was concentrated under reduced pressure. The residue was purified by flash chromatography with petroleum ether/ethyl acetate (40/60, v/v) to afford 5-(5-fluoro-2-methyl-4-nitrophenoxy)-1-methyl-1,2,3-benzotriazole (820.1 mg, 68%) as a yellow solid. LCMS (ESI, m/z): [M+H]⁺=303.1.

### Step 2. Synthesis of 2-fluoro-5-methyl-4-[(1-methyl-1,2,3-benzotriazol-5-yl)oxy]aniline

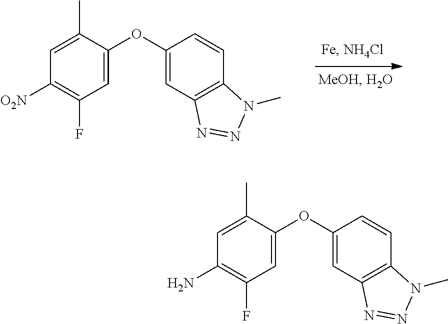 [SMILES: Cc1cc([N+](=O)[O-])c(F)cc1Oc1ccc2c(c1)nnn2C ; Cc1cc(N)c(F)cc1Oc1ccc2c(c1)nnn2C]

To a solution of 5-(5-fluoro-2-methyl-4-nitrophenoxy)-1-methyl-1,2,3-benzotriazole (800.0 mg, 2.65 mmol) in MeOH (10.0 mL) and H₂O (3.0 mL) was added Fe (1484.6 mg, 26.51 mmol) and NH₄Cl (140.5 mg, 2.65 mmol) at room temperature. The resulting mixture was stirred at 80° C. for 2 h. After the reaction was completed, the resulting mixture was filtered. The filtrate was concentrated under vacuum. The residue was purified by flash chromatography with CH₂Cl₂/MeOH (92/8, v/v) to afford 2-fluoro-5-methyl-4-[(1-methyl-1,2,3-benzotriazol-5-yl)oxy]aniline (440.0 mg, 58%) as a yellow solid. LCMS (ESI, m/z): [M+H]⁺=273.2.

### Step 3. Synthesis of 6-chloro-N-{2-fluoro-5-methyl-4-[(1-methyl-1,2,3-benzotriazol-5-yl)oxy]phenyl}pyrido[3,4-d]pyrimidin-4-amine

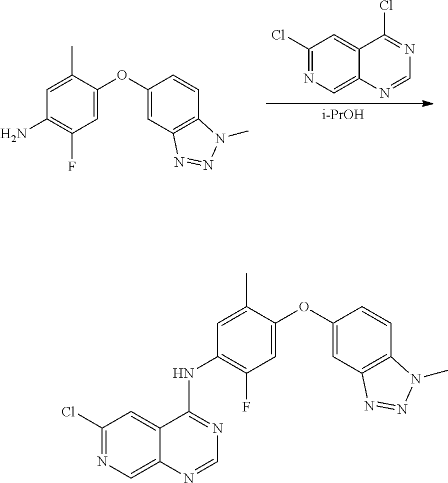 [SMILES: "i-PrOH" ; Cc1cc(N)c(F)cc1Oc1ccc2c(c1)nnn2C ; Clc1cc2c(Cl)ncnc2cn1 ; Cc1cc(Nc2ncnc3cnc(Cl)cc23)c(F)cc1Oc1ccc2c(c1)nnn2C]

To a solution of 2-fluoro-5-methyl-4-[(1-methyl-1,2,3-benzotriazol-5-yl)oxy]aniline (350.0 mg, 1.28 mmol) in i-PrOH (10.0 mL) was added 4,6-dichloro-3H,4H-pyrido[3,4-d]pyrimidine (259.7 mg, crude) at room temperature. The resulting mixture was stirred at room temperature for 16 h. After the reaction was completed, the resulting mixture was concentrated reduced pressure. The residue was purified by flash chromatography with CH₂Cl₂/MeOH (85/15, v/v) to afford 6-chloro-N-{2-fluoro-5-methyl-4-[(1-methyl-1,2,3-benzotriazol-5-yl)oxy]phenyl}pyrido[3,4-d]pyrimidin-4-amine (200.0 mg, 32%) as a yellow solid. LCMS (ESI, m/z): [M+H]⁺=436.1.

### Step 4. Synthesis of (3E)-3-[2-(dimethylamino)ethylidene]-1-[4-({2-fluoro-5-methyl-4-[(1-methyl-1,2,3-benzotriazol-5-yl)oxy]phenyl}amino)pyrido[3,4-d]pyrimidin-6-yl]-4-methylpyrrolidin-2-one

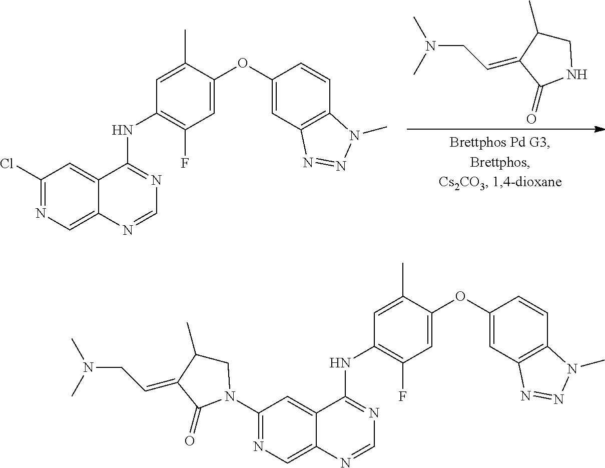

To a solution of 6-chloro-N-{2-fluoro-5-methyl-4-[(1-methyl-1,2,3-benzotriazol-5-yl)oxy]phenyl}pyrido[3,4-d]pyrimidin-4-amine (180.0 mg, 0.41 mmol) in dioxane (5.0 mL) was added (E)-3-(2-(dimethylamino)ethylidene)-4-methylpyrrolidin-2-one (69.4 mg, 0.41 mmol), BrettPhos Pd G3 (37.4 mg, 0.04 mmol), BrettPhos (44.3 mg, 0.08 mmol) and Cs₂CO₃ (403.7 mg, 1.24 mmol) at room temperature under N₂. The resulting mixture was stirred at 100° C. for 2 h. After the reaction was completed, the resulting mixture was filtered. The filtrate was concentrated reduced pressure. The residue was purified by flash chromatography with DCM/MeOH (90/10, v/v) to afford (3E)-3-[2-(dimethylamino)ethylidene]-1-[4-({2-fluoro-5-methyl-4-[(1-methyl-1,2,3-benzotriazol-5-yl)oxy]phenyl}amino)pyrido[3,4-d]pyrimidin-6-yl]-4-methylpyrrolidin-2-one (100.0 mg, 40%) as a yellow solid. LCMS (ESI, m/z): [M+H]⁺=568.3.

### Step 5. Synthesis of (3E,4R)-3-[2-(dimethylamino)ethylidene]-1-[4-({2-fluoro-5-methyl-4-[(1-methyl-1,2,3-benzotriazol-5-yl)oxy]phenyl}amino)pyrido[3,4-d]pyrimidin-6-yl]-4-methylpyrrolidin-2-one and (3E,4S)-3-[2-(dimethylamino)ethylidene]-1-[4-({2-fluoro-5-methyl-4-[(1-methyl-1,2,3-benzotriazol-5-yl)oxy]phenyl}amino)pyrido[3,4-d]pyrimidin-6-yl]-4-methylpyrrolidin-2-one (Compound 131 and Compound 132)

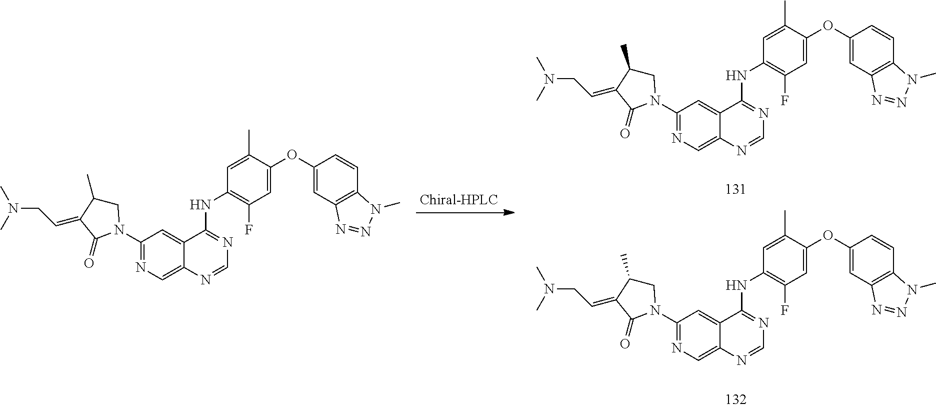

The racemic of (3E)-3-[2-(dimethylamino)ethylidene]-1-[4-({2-fluoro-5-methyl-4-[(1-methyl-1,2,3-benzotriazol-5-yl)oxy]phenyl}amino)pyrido[3,4-d]pyrimidin-6-yl]-4-methylpyrrolidin-2-one (100.0 mg, 0.17 mmol) was separated by Prep-Chiral-HPLC with the following conditions (Column: CHIRAL ART Amylose-SA, 2×25 cm, 5 μm; Mobile Phase A: Hex (0.5% 2M NH₃-MeOH)-HPLC, Mobile Phase B: EtOH: DCM=1: 1-HPLC; Flow rate: 20 mL/min; Gradient: 30% B to 30% B in 21 min; Wave Length: 220/254 nm; RT1(min): 14.47; RT2(min): 17.85) to afford (3E)-3-[2-(dimethylamino)ethylidene]-1-[4-({2-fluoro-5-methyl-4-[(1-methyl-1,2,3-benzotriazol-5-yl)oxy]phenyl}amino)pyrido[3,4-d]pyrimidin-6-yl]-4-methylpyrrolidin-2-one Enantiomer 1 (RT1 14.47 min, 23.1 mg, 46%) and (3E)-3-[2-(dimethylamino)ethylidene]-1-[4-({2-fluoro-5-methyl-4-[(1-methyl-1,2,3-benzotriazol-5-yl)oxy]phenyl}amino)pyrido[3,4-d]pyrimidin-6-yl]-4-methylpyrrolidin-2-one Enantiomer 2 (RT2 17.85 min, 35.2 mg, 70%) as a light yellow solid. The absolute stereochemistry of Enantiomers 1 and 2 was not assigned. The two enantiomeric structures that could be obtained from chiral separation of the enantiomeric mixture as described above are shown as Compounds 131 and 132 in Table 1.

(3E)-3-[2-(dimethylamino)ethylidene]-1-[4-({2-fluoro-5-methyl-4-[(1-methyl-1,2,3-benzotriazol-5-yl)oxy]phenyl}amino)pyrido[3,4-d]pyrimidin-6-yl]-4-methylpyrrolidin-2-one Enantiomer 1: RT1(min): 14.47; LCMS (ESI, m/z): [M+H]⁺=568.5. ¹H NMR (400 MHz, DMSO-d₆): δ 10.33 (s, 1H), 9.18 (s, 1H), 9.09 (s, 1H), 8.55 (s, 1H), 7.95 (d, J=8.8 Hz, 1H), 7.51-7.48 (m, 2H), 7.43-7.40 (m, 1H), 6.88-6.85 (m, 1H), 6.54-6.51 (m, 1H), 4.33 (s, 3H), 4.27-4.23 (m, 1H), 3.83-3.81 (m, 1H), 3.31-3.23 (m, 2H), 3.17-3.13 (m, 1H), 2.26-2.15 (m, 9H), 1.29-1.20 (m, 3H).

(3E)-3-[2-(dimethylamino)ethylidene]-1-[4-({2-fluoro-5-methyl-4-[(1-methyl-1,2,3-benzotriazol-5-yl)oxy]phenyl}amino)pyrido[3,4-d]pyrimidin-6-yl]-4-methylpyrrolidin-2-one Enantiomer 2: RT2(min): 17.85; LCMS (ESI, m/z): [M+H]⁺=568.4. ¹H NMR (400 MHz, DMSO-d₆): δ 10.34 (s, 1H), 9.18 (s, 1H), 9.10 (s, 1H), 8.55 (s, 1H), 7.95 (d, J=8.8 Hz, 1H), 7.51-7.48 (m, 2H), 7.43-7.40 (m, 1H), 6.89-6.86 (m, 1H), 6.54-6.51 (m, 1H), 4.33-4.24 (m, 4H), 3.84-3.82 (m, 1H), 3.52-3.45 (m, 2H), 2.37 (s, 6H), 2.26 (s, 3H), 1.26-1.24 (m, 3H).

### Example S116: Synthesis of (E)-1-(4-((4-([1,2,4]triazolo[1,5-a]pyridin-7-yloxy)-5-chloro-2-fluorophenyl)amino)pyrido[3,4-d]pyrimidin-6-yl)-3-(2-(dimethylamino)ethylidene)pyrrolidin-2-one (Compound 133)

### Step 1. Synthesis of 4,6-dichloropyrido[3,4-d]pyrimidine

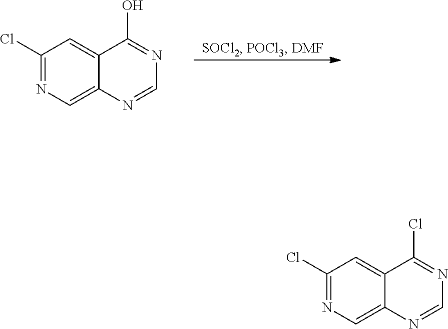

To a solution of 6-chloropyrido[3,4-d]pyrimidin-4-ol (500.0 mg, 2.76 mmol) in SOCl₂ (8.0 mL) was added POCl₃ (1.0 mL) and DMF (0.1 mL) at room temperature under N₂. The resulting mixture was stirred at 80° C. for 2 h. After the reaction was completed, the resulting mixture was concentrated under reduced pressure to afford 4,6-dichloropyrido[3,4-d]pyrimidine (540.0 mg, crude) as a yellow solid.

### Step 2. Synthesis of N-(4-([1,2,4]triazolo[1,5-a]pyridin-7-yloxy)-5-chloro-2-fluorophenyl)-6-chloropyrido[3,4-d]pyrimidin-4-amine

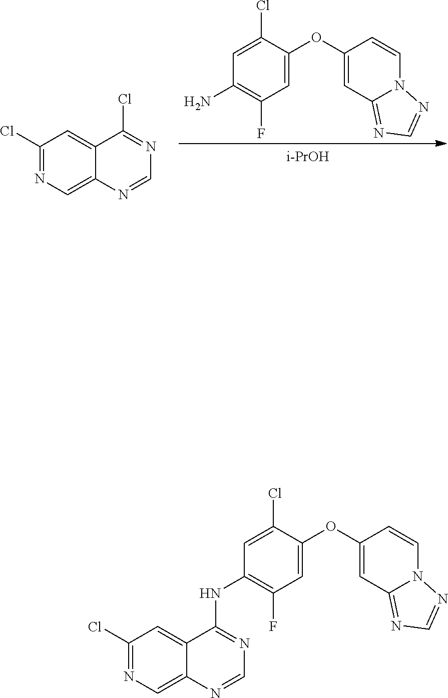 [SMILES: Clc1cc2c(Cl)ncnc2cn1 ; "i-PrOH" ; Nc1cc(Cl)c(Oc2ccn3ncnc3c2)cc1F ; Fc1cc(Oc2ccn3ncnc3c2)c(Cl)cc1Nc1ncnc2cnc(Cl)cc12]

To a solution of 4-([1,2,4]triazolo[1,5-a]pyridin-7-yloxy)-5-chloro-2-fluoroaniline (750.0 mg, 2.70 mmol) in isopropanol (15.0 mL) was added 4,6-dichloropyrido[3,4-d]pyrimidine (540.0 mg, crude) at room temperature. The resulting mixture was stirred at 30° C. for 16 h. After the reaction was completed, the resulting mixture was concentrated under reduced pressure. The residue was purified by flash column with dichloromethane/methanol (85/15, v/v) to afford N-(4 ([1,2,4]triazolo[1,5-a]pyridin-7-yloxy)-5-chloro-2-fluorophenyl)-6-chloropyrido[3,4-d]pyrimidin-4-amine (600.0 mg, 50%) as a yellow solid. LCMS (ESI, m/z): [M+H]⁺=442.0.

### Step 3. Synthesis of (E)-1-(4-((4-([1,2,4]triazolo[1,5-a]pyridin-7-yloxy)-5-chloro-2-fluorophenyl)amino)pyrido[3,4-d]pyrimidin-6-yl)-3-(2-(dimethylamino)ethylidene)pyrrolidin-2-one (Compound 133)

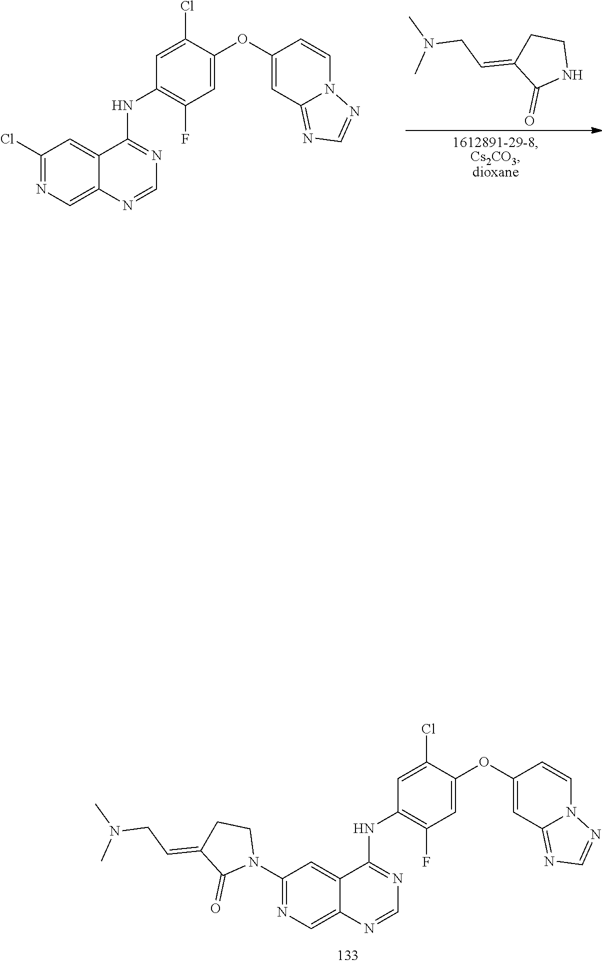 [SMILES: Fc1cc(Oc2ccn3ncnc3c2)c(Cl)cc1Nc1ncnc2cnc(Cl)cc12 ; CN(C)C/C=C1\CCNC1=O ; "1612891-29-8,Cs2CO3,dioxane" ; CN(C)C/C=C1\CCN(c2cc3c(Nc4cc(Cl)c(Oc5ccn6ncnc6c5)cc4F)ncnc3cn2)C1=O]

To a solution of N-(4-([1,2,4]triazolo[1,5-a]pyridin-7-yloxy)-5-chloro-2-fluorophenyl)-6-chloropyrido[3,4-d]pyrimidin-4-amine (300.0 mg, 0.68 mmol) in 1,4-dioxane (10.0 mL) was added (E)-3-(2-(dimethylamino)ethylidene)pyrrolidin-2-one (104.6 mg, 0.68 mmol), Cs₂CO₃ (663.0 mg, 2.04 mmol) and Pd-PEPPSI-IPentCl 2-methylpyridine (o-picoline) (114.1 mg, 0.13 mmol) at room temperature under N₂. The resulting mixture was stirred at 100° C. for 16 h under N₂. After the reaction was completed, the resulting mixture was filtered. The filtrate was concentrated under reduced pressure. The residue was purified by flash column with dichloromethane/methanol (90/10, v/v) and then purified by Prep-HPLC with the following conditions: (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 28% B to 40% B in 8 min; Wave Length: 254 nm) to afford (E)-1-(4-((4-([1,2,4]triazolo[1,5-a]pyridin-7-yloxy)-5-chloro-2-fluorophenyl)amino)pyrido[3,4-d]pyrimidin-6-yl)-3-(2-(dimethylamino)ethylidene)pyrrolidin-2-one (Compound 133) (11.6 mg, 3%) as a white solid. LCMS (ESI, m/z): [M+H]⁺=560.2. ¹H NMR (400 MHz, CD₃OD): δ 9.13 (s, 1H), 9.06 (s, 1H), 8.82 (d, J=7.6 Hz, 1H), 8.55 (s, 1H), 8.35 (s, 1H), 8.03 (d, J=6.8 Hz, 1H), 7.44 (d, J=10.0 Hz, 1H), 7.15-7.13 (m, 1H), 7.06 (d, J=2.0 Hz, 1H), 6.74-6.71 (m, 1H), 4.30-4.27 (m, 2H), 3.25 (d, J=6.8 Hz, 2H), 2.97-2.92 (m, 2H), 2.36 (s, 6H).

### Example S117: Synthesis of (R,E)-3-(2-(dimethylamino)ethylidene)-1-(4-((3-fluoro-4-((1-methyl-1H-benzo[d][1,2,3]triazol-5 yl)oxy)phenyl)amino)pyrido[3,4-d]pyrimidin-6-yl)-4-methylpyrrolidin-2-one and (S,E)-3-(2-(dimethylamino)ethylidene)-1-(4-((3-fluoro-4-((1-methyl-1H-benzo[d][1,2,3]triazol-5 yl)oxy)phenyl)amino)pyrido[3,4-d]pyrimidin-6-yl)-4-methylpyrrolidin-2-one (Compound 134 and Compound 135)

### Step 1. Synthesis of Tert-butyl 4-methyl-2-oxopyrrolidine-1-carboxylate

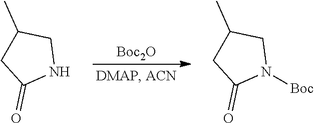

To a solution of 4-methylpyrrolidin-2-one (10.0 g, 100.88 mmol) in ACN (100.0 mL) was added DMAP (1.2 g, 10.09 mmol) and Boc₂O (26.4 g, 121.10 mmol) at room temperature.

The resulting mixture was stirred at room temperature for 4 h. After the reaction was completed, the resulting mixture was concentrated under reduced pressure. The residue was purified by reverse phase flash chromatography with CH₃CN/H₂O (40/60, v/v) to afford tert-butyl 4-methyl-2-oxopyrrolidine-1-carboxylate (8.0 g, 40%) as a brown yellow oil. LCMS (ESI, m/z): [M+H]⁺=200.1.

### Step 2. Synthesis of Tert-butyl 3-{2-[(tert-butyldimethylsilyl)oxy]-1-hydroxyethyl}-4-methyl-2-oxopyrrolidine-1-carboxylate

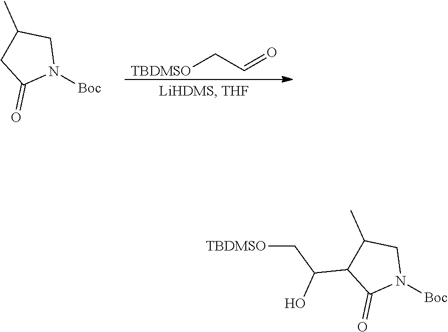

To a solution of tert-butyl 4-methyl-2-oxopyrrolidine-1-carboxylate (5.0 g, 25.09 mmol) in THF (100.0 mL) was added LiHMDS (51.0 mL, 1 mol/L) at −78° C. under N₂. The mixture was stirred at −78° C. for 2 h under N₂. Then a solution of 2-[(tert-butyldimethylsilyl)oxy]acetaldehyde (5.3 g, 30.42 mmol) in THF (5.0 mL) was added dropwise to the mixture at −78° C. The mixture was stirred at −78° C. for additional 2 h under N₂. After the reaction was completed, the reaction mixture was quenched with NH₄Cl (aq.) and then extracted with ethyl acetate. The combined organic layer was washed with brine, dried over anhydrous sodium sulfate and filtered. The filtrate was concentrated under vacuum to afford tert-butyl 3-{2-[(tert-butyldimethylsilyl)oxy]-1-hydroxyethyl}-4-methyl-2-oxopyrrolidine-1-carboxylate (10.2 g, crude) as a yellow oil. LCMS (ESI, m/z): [M+H]⁺=374.2.

### Step 3. Synthesis of Tert-butyl 3-{2-[(tert-butyldimethylsilyl)oxy]-1-(methanesulfonyloxy)ethyl}-4-methyl-2-oxopyrrolidine-1-carboxylate

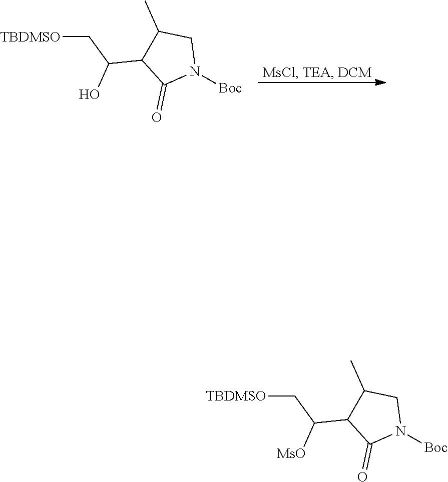 [SMILES: CC1CN(C(=O)OC(C)(C)C)C(=O)C1C(O)CO[Si](C)(C)C(C)(C)C ; "MsCl, TEA, DCM" ; CC1CN(C(=O)OC(C)(C)C)C(=O)C1C(CO[Si](C)(C)C(C)(C)C)OS(C)(=O)=O]

To a solution of tert-butyl 3-{2-[(tert-butyldimethylsilyl)oxy]-1-hydroxyethyl}-4-methyl-2-oxopyrrolidine-1-carboxylate (2.0 g, crude) in CH₂Cl₂ (50.0 mL) was added TEA (2.2 g, 21.42 mmol) and MsCl (1.2 g, 10.71 mmol) at 0° C. The resulting mixture was stirred at room temperature for 16 h. After the reaction was completed, the mixture was diluted with H₂O and extracted with CH₂Cl₂. The combined organic layer was washed with brine, dried over anhydrous sodium sulfate and filtered. The filtrate was concentrated under vacuum to afford tert-butyl 3-{2-[(tert-butyldimethylsilyl)oxy]-1-(methanesulfonyloxy)ethyl}-4-methyl-2-oxopyrrolidine-1-carboxylate (4.1 g, crude) as a brown oil. LCMS (ESI, m/z): [M+H]⁺=452.2.

### Step 4. Synthesis of Tert-butyl (3E)-3-{2-[(tert-butyldimethylsilyl)oxy]ethylidene}-4-methyl-2-oxopyrrolidine-1-carboxylate

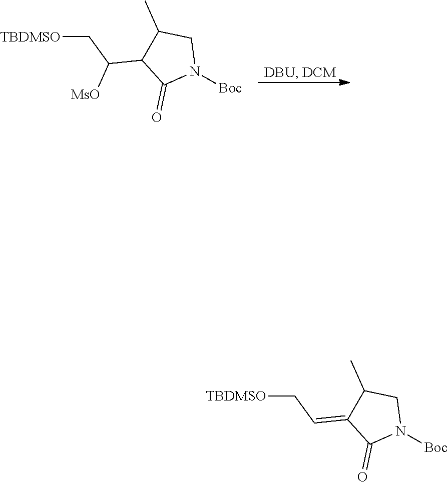

To a solution of tert-butyl 3-{2-[(tert-butyldimethylsilyl)oxy]-1-(methanesulfonyloxy)ethyl}-4-methyl-2-oxopyrrolidine-1-carboxylate (40.0 g, crude) in DCM (500.0 mL) was added DBU (53.9 g, 354.21 mmol) at 0° C. The resulting mixture was stirred at room temperature for 2 h. After the reaction was completed, the mixture was diluted with H₂O and extracted with CH₂Cl₂. The combined organic layer was washed with brine, dried over anhydrous sodium sulfate and filtered. The filtrate was concentrated under vacuum. The residue was purified by flash column chromatography with petroleum ether/ethyl acetate (90/10, v/v) to afford tert-butyl (3E)-3-{2-[(tert-butyldimethylsilyl)oxy]ethylidene}-4-methyl-2-oxopyrrolidine-1-carboxylate (10.0 g, 31%) as a yellow oil: [M+H]⁺=356.2.

### Step 5. Synthesis of Tert-butyl (3E)-3-(2-hydroxyethylidene)-4-methyl-2-oxopyrrolidine-1-carboxylate

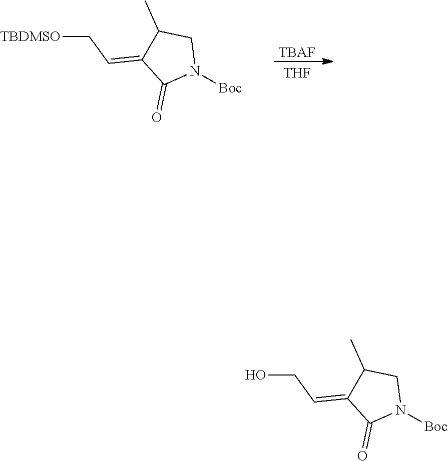 [SMILES: CC1CN(C(=O)OC(C)(C)C)C(=O)/C1=C/CO[Si](C)(C)C(C)(C)C ; "TBAF" ; CC1CN(C(=O)OC(C)(C)C)C(=O)/C1=C/CO ; "THF"]

To a mixture of tert-butyl (3E)-3-{2-[(tert-butyldimethylsilyl)oxy]ethylidene}-4-methyl-2-oxopyrrolidine-1-carboxylate (1.7 g, 4.78 mmol) in tetrahydrofuran (20.0 mL) was added TBAF (6.0 mL, 1 mol/L) at room temperature. The resulting mixture was stirred at room temperature for 2 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (50/50, v/v) to afford tert-butyl (3E)-3-(2-hydroxyethylidene)-4-methyl-2-oxopyrrolidine-1-carboxylate (450.0 mg, 39%) as a yellow oil. LCMS (ESI, m/z): [M+H]⁺=242.1.

### Step 6. Synthesis of Tert-butyl (3E)-3-[2-(methanesulfonyloxy)ethylidene]-4-methyl-2-oxopyrrolidine-1-carboxylate

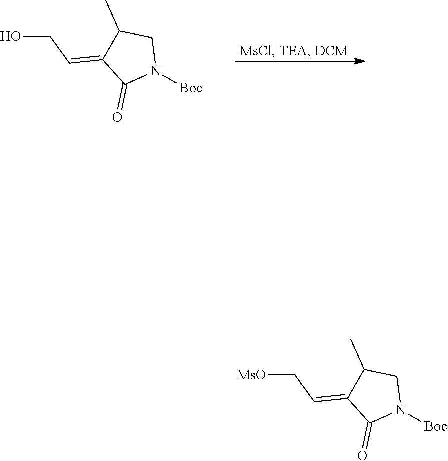 [SMILES: CC1CN(C(=O)OC(C)(C)C)C(=O)/C1=C/CO ; "MsCl, TEA, DCM" ; CC1CN(C(=O)OC(C)(C)C)C(=O)/C1=C/COS(C)(=O)=O]

To a solution of tert-butyl (3E)-3-(2-hydroxyethylidene)-4-methyl-2-oxopyrrolidine-1-carboxylate (1.0 g, 4.14 mmol) in DCM (15.0 mL) was added TEA (1.3 g, 12.43 mmol) and MsCl (0.7 g, 6.22 mmol) at 0° C. The resulting mixture was stirred at room temperature for 16 h.

After the reaction was completed, the mixture was diluted with H₂O and extracted with CH₂Cl₂. The combined organic layer was washed with brine, dried over anhydrous sodium sulfate and filtered. The filtrate was concentrated under vacuum to afford tert-butyl (3E)-3-[2-(methanesulfonyloxy)ethylidene]-4-methyl-2-oxopyrrolidine-1-carboxylate (1.0 g, crude) as a yellow oil. LCMS (ESI, m/z): [M+H]⁺=320.1.

### Step 7. Synthesis of Tert-butyl (3E)-3-[2-(dimethylamino)ethylidene]-4-methyl-2-oxopyrrolidine-1-carboxylate

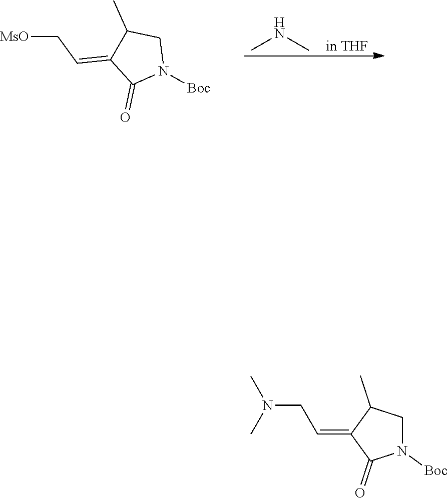

To a solution of tert-butyl (3E)-3-[2-(methanesulfonyloxy)ethylidene]-4-methyl-2-oxopyrrolidine-1-carboxylate (2.0 g, crude) in THF (20.0 mL) was added a solution of dimethylamine in THF (8.0 mL, 2 mol/L) at room temperature. The resulting mixture was stirred at 60° C. for 16 h. After the reaction was completed, the mixture was concentrated under reduced pressure. The residue was purified by flash column chromatography with CH₂Cl₂/MeOH (90/10, v/v) to afford tert-butyl (3E)-3-[2-(dimethylamino)ethylidene]-4-methyl-2-oxopyrrolidine-1-carboxylate (700.0 mg, 41%) as a yellow oil. LCMS (ESI, m/z): [M+H]⁺=269.2.

### Step 8. Synthesis of (E)-3-(2-(dimethylamino)ethylidene)-4-methylpyrrolidin-2-one 2,2,2-trifluoroacetate

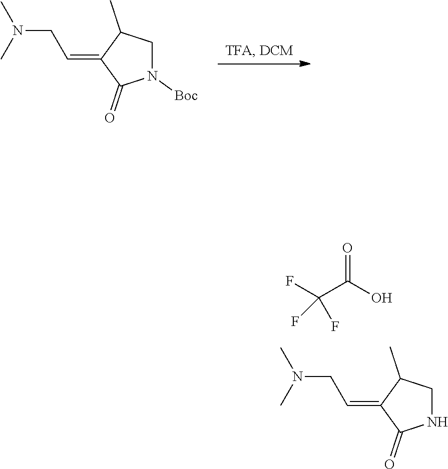

To a solution of tert-butyl (3E)-3-[2-(dimethylamino)ethylidene]-4-methyl-2-oxopyrrolidine-1-carboxylate (150.0 mg, 0.56 mmol) in DCM (5.0 mL) was added TFA (1.0 mL) at room temperature. The resulting mixture was stirred at room temperature for 2 h. After the reaction was completed, the mixture was concentrated under reduced pressure to afford (E)-3-(2-(dimethylamino)ethylidene)-4-methylpyrrolidin-2-one 2,2,2-trifluoroacetate (210.0 mg, crude) as a yellow oil. LCMS (ESI, m/z): [M+H]⁺=169.1.

### Step 9. Synthesis of 5-(2-fluoro-4-nitrophenoxy)-1-methyl-1H-benzo[d][1,2,3]triazole

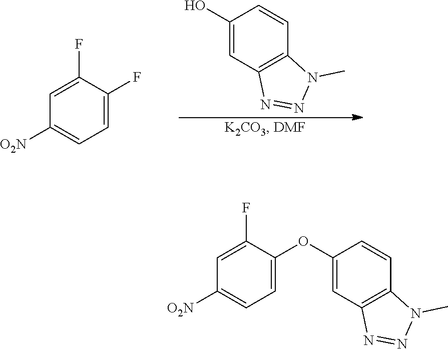

To a solution of 1-methyl-1,2,3-benzotriazol-5-ol (500.0 mg, 3.35 mmol) in DMF (10.0 mL) was added 1,2-difluoro-4-nitrobenzene (532.6 mg, 3.35 mmol) and K₂CO₃ (1.4 g, 10.06 mmol) at room temperature. The resulting mixture was stirred at 100° C. for 2 h. After the reaction was completed, the resulting mixture was diluted with H₂O and then filtered. The solid was washed with H₂O and then collected to afford 5-(2-fluoro-4-nitrophenoxy)-1-methyl-1H-benzo[d][1,2,3]triazole (700.0 mg, crude) as a yellow solid. LCMS (ESI, m/z): [M+H]⁺=289.1.

### Step 10. Synthesis of 3-fluoro-4-((1-methyl-1H-benzo[d][1,2,3]triazol-5-yl)oxy)aniline

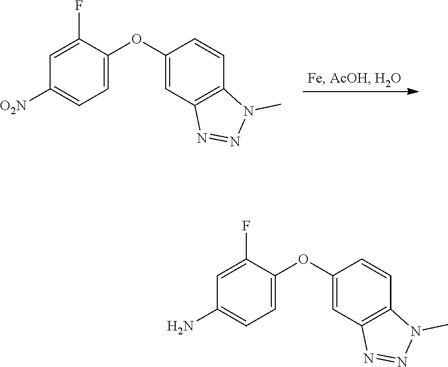

To a solution of 5-(2-fluoro-4-nitrophenoxy)-1-methyl-1H-benzo[d][1,2,3]triazole (700.0 mg, crude) in AcOH (20.0 mL) and H₂O (1.0 mL) was added Fe (723.3 mg, 12.95 mmol) at room temperature. The resulting mixture was stirred at room temperature for 16 h. After the reaction was completed, the resulting mixture was filtered. The filtrate was concentrated under vacuum. The residue was purified by flash column chromatography with petroleum ether/ethyl acetate (50/50, v/v) to afford 3-fluoro-4-((1-methyl-1H-benzo[d][1,2,3]triazol-5-yl)oxy)aniline (420.0 mg, 67%) as a yellow solid. LCMS (ESI, m/z): [M+H]⁺=259.1.

### Step 11. Synthesis of 6-chloro-N-(3-fluoro-4-((1-methyl-1H-benzo[d][1,2,3]triazol-5-yl)oxy)phenyl)pyrido[3,4-d]pyrimidin-4-amine

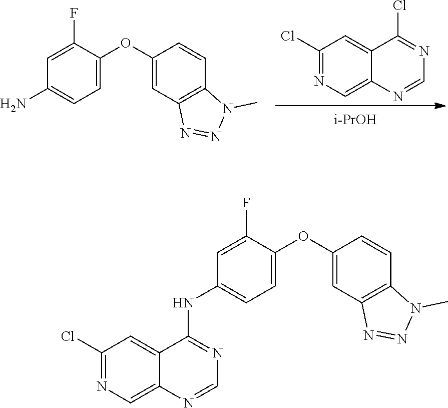 [SMILES: "i-PrOH" ; Cn1nnc2cc(Oc3ccc(N)cc3F)ccc21 ; Clc1cc2c(Cl)ncnc2cn1 ; Cn1nnc2cc(Oc3ccc(Nc4ncnc5cnc(Cl)cc45)cc3F)ccc21]

To a solution of 3-fluoro-4-((1-methyl-1H-benzo[d][1,2,3]triazol-5-yl)oxy)aniline (214.4 mg, 0.83 mmol) in isopropyl alcohol (10.0 mL) was added 4,6-dichloropyrido[3,4-d]pyrimidine (333.0 mg, crude) at room temperature. The resulting mixture was stirred at room temperature for 1 h. After the reaction was completed, the pH value of the mixture was adjusted to 7 with NaHCO₃ (aq.). The mixture was extracted with EtOAc. The combined organic layers were washed with brine and dried over anhydrous Na₂SO₄. After filtration, the filtrate was concentrated under vacuum. The residue was purified by flash column chromatography with CH₂Cl₂/MeOH (20/1, v/v) to afford 6-chloro-N-(3-fluoro-4-((1-methyl-1H-benzo[d][1,2,3]triazol-5-yl)oxy)phenyl)pyrido[3,4-d]pyrimidin-4-amine (174.7 mg, 50%) as a yellow solid. LCMS (ESI, m/z): [M+H]⁺=422.1.

### Step 12. Synthesis of (E)-3-(2-(dimethylamino)ethylidene)-1-(4-((3-fluoro-4-((1-methyl-1H-benzo[d][1,2,3]triazol-5-yl)oxy)phenyl)amino)pyrido[3,4-d]pyrimidin-6-yl)-4-methylpyrrolidin-2-one

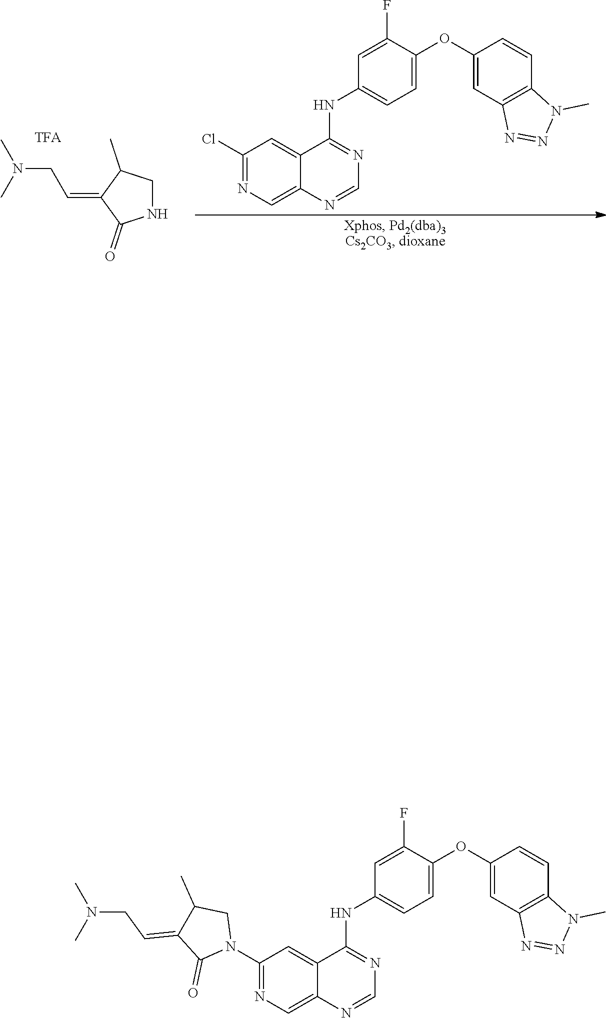

To a solution of 6-chloro-N-(3-fluoro-4-((1-methyl-1H-benzo[d][1,2,3]triazol-5-yl)oxy)phenyl)pyrido[3,4-d]pyrimidin-4-amine (105.0 mg, 0.25 mmol) in dioxane (5.0 mL) was added (E)-3-(2-(dimethylamino)ethylidene)-4-methylpyrrolidin-2-one 2,2,2-trifluoroacetate (41.7 mg, crude), Cs₂CO₃ (403.4 mg, 1.24 mmol), XPhos (23.6 mg, 0.05 mmol) and Pd₂(dba)₃ (22.7 mg, 0.03 mmol) at room temperature. The resulting mixture was stirred at 100° C. for 1 h under N₂. After the reaction was completed, the resulting mixture was filtered. The filtrate was concentrated under vacuum. The residue was purified by flash column chromatography with CH₂Cl₂/MeOH (90/10, v/v) to afford (E)-3-(2-(dimethylamino)ethylidene)-1-(4-((3-fluoro-4-((1-methyl-1H-benzo[d][1,2,3]triazol-5-yl)oxy)phenyl)amino)pyrido[3,4-d]pyrimidin-6-yl)-4-methylpyrrolidin-2-one (70.0 mg, 50%) as a yellow solid. LCMS (ESI, m/z): [M+H]⁺=554.2.

### Step 13. Synthesis of (R,E)-3-(2-(dimethylamino)ethylidene)-1-(4-((3-fluoro-4-((1-methyl-1H-benzo[d][1,2,3]triazol-5-yl)oxy)phenyl)amino)pyrido[3,4-d]pyrimidin-6-yl)-4-methylpyrrolidin-2-one and (S,E)-3-(2-(dimethylamino)ethylidene)-1-(4-((3-fluoro-4-((1-methyl-1H-benzo[d][1,2,3]triazol-5-yl)oxy)phenyl)amino)pyrido[3,4-d]pyrimidin-6-yl)-4-methylpyrrolidin-2-one (Compound 134 and Compound 135)

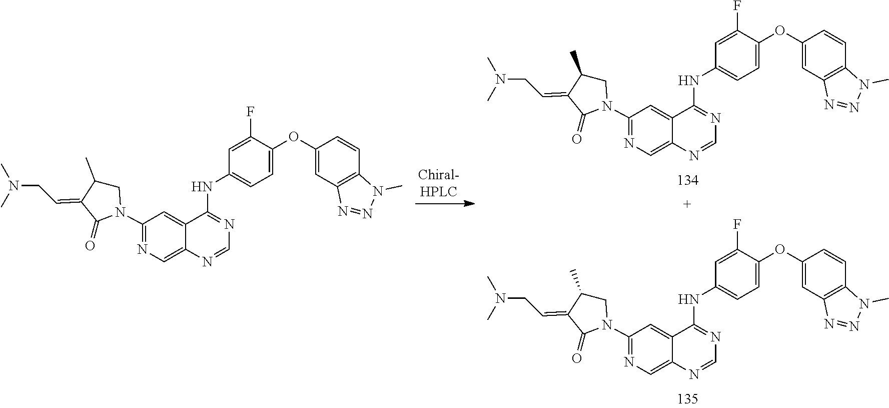

The racemic product of (E)-3-(2-(dimethylamino)ethylidene)-1-(4-((3-fluoro-4-((1-methyl-1H-benzo[d][1,2,3]triazol-5-yl)oxy)phenyl)amino)pyrido[3,4-d]pyrimidin-6-yl)-4-methylpyrrolidin-2-one (70.0 mg, 0.13 mmol) was separated by Prep-Chiral-HPLC with the following conditions (Column: CHIRAL ART Amylose-SA, 2×25 cm, 5 μm; Mobile Phase A: Hex (0.5% 2M NH₃-MeOH)-HPLC, Mobile Phase B: MeOH: DCM=1: 1-HPLC; Flow rate: 20 mL/min; Gradient: 30% B to 30% B in 17 min; Wave Length: 220/254 nm; RT1(min): 12.00; RT2(min): 15.10) to afford (E)-3-(2-(dimethylamino)ethylidene)-1-(4-((3-fluoro-4-((1-methyl-1H-benzo[d][1,2,3]triazol-5-yl)oxy)phenyl)amino)pyrido[3,4-d]pyrimidin-6-yl)-4-methylpyrrolidin-2-one Enantiomer 1 (RT1 12.00 min, 23.6 mg, 67%) as a yellow solid and (E)-3-(2-(dimethylamino)ethylidene)-1-(4-((3-fluoro-4-((1-methyl-1H-benzo[d][1,2,3]triazol-5-yl)oxy)phenyl)amino)pyrido[3,4-d]pyrimidin-6-yl)-4-methylpyrrolidin-2-one Enantiomer 2 (RT2 15.10 min, 23.4 mg, 67%) as a yellow solid. The absolute stereochemistry of Enantiomers 1 and 2 was not assigned. The two enantiomeric structures that could be obtained from chiral separation of the enantiomeric mixture as described above are shown as Compounds 134 and 135 in Table 1.

(E)-3-(2-(dimethylamino)ethylidene)-1-(4-((3-fluoro-4-((1-methyl-1H-benzo[d][1,2,3]triazol-5-yl)oxy)phenyl)amino)pyrido[3,4-d]pyrimidin-6-yl)-4-methylpyrrolidin-2-one Enantiomer 1: RT1(min): 12.00; LCMS (ESI, m/z): [M+H]⁺=554.2. ¹H NMR (400 MHz, DMSO-d₆): δ 10.41 (s, 1H), 9.20 (s, 1H), 9.10 (s, 1H), 8.69 (s, 1H), 8.13-8.10 (m, 1H), 7.91 (d, J=8.8 Hz, 1H), 7.72 (d, J=8.4 Hz, 1H), 7.47-7.41 (m, 2H), 7.31-7.26 (m, 1H), 6.55-6.52 (m, 1H), 4.32 (s, 3H), 4.28-4.23 (m, 1H), 3.82-3.80 (m, 1H), 3.38-3.21 (m, 2H), 3.15-3.11 (m, 1H), 2.22 (s, 6H), 1.25 (d, J=6.8 Hz, 3H).

(E)-3-(2-(dimethylamino)ethylidene)-1-(4-((3-fluoro-4-((1-methyl-1H-benzo[d][1,2,3]triazol-5-yl)oxy)phenyl)amino)pyrido[3,4-d]pyrimidin-6-yl)-4-methylpyrrolidin-2-one Enantiomer 2: RT2(min): 15.10; LCMS (ESI, m/z): [M+H]⁺=554.2. ¹H NMR (400 MHz, DMSO-d₆): δ 10.41 (s, 1H), 9.20 (s, 1H), 9.11 (s, 1H), 8.69 (s, 1H), 8.13-8.10 (m, 1H), 7.95-7.90 (m, 1H), 7.72 (d, J=8.4 Hz, 1H), 7.47-7.41 (m, 2H), 7.35-7.26 (m, 1H), 6.54-6.49 (m, 1H), 4.32 (s, 3H), 4.26-4.24 (m, 1H), 3.83-3.80 (m, 1H), 3.27-3.18 (m, 3H), 2.26 (s, 6H), 1.25 (d, J=6.8 Hz, 3H).

### Example S118: Synthesis of (3E,4R)-3 [2-(dimethylamino)ethylidene]-4-methyl-1-[4-({4-[(1-methyl-1,2,3-benzotriazol-5 yl)oxy]phenyl}amino)pyrido[3,4-d]pyrimidin-6-yl]pyrrolidin-2-one and (3E,4S)-3-[2-(dimethylamino)ethylidene]-4-methyl-1-[4-({4-[(1-methyl-1,2,3-benzotriazol-5-yl)oxy]phenyl}amino)pyrido[3,4-d]pyrimidin-6-yl]pyrrolidin-2-one (Compound 136 and Compound 137)

### Step 1. Synthesis of 1-methyl-5-(4-nitrophenoxy)-1,2,3-benzotriazole

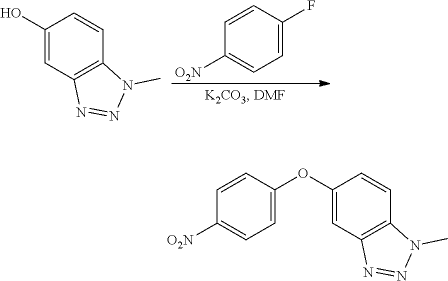

To a solution of 1-methyl-1,2,3-benzotriazol-5-ol (500.0 mg, 3.35 mmol) in DMF (10.0 mL) was added 4-fluoronitrobenzene (473.0 mg, 3.35 mmol) and K₂CO₃ (1.4 g, 10.06 mmol) at room temperature. The resulting mixture was stirred at 100° C. for 2 h. After the reaction was completed, the resulting mixture was diluted with H₂O and then filtered. The solid was washed with water and then collected to afford 1-methyl-5-(4-nitrophenoxy)-1,2,3-benzotriazole (700.0 mg, crude) as a yellow solid. LCMS (ESI, m/z): [M+H]⁺=271.1.

### Step 2. Synthesis of 4-[(1-methyl-1,2,3-benzotriazol-5-yl)oxy]aniline

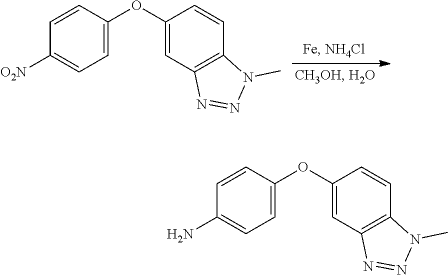 [SMILES: Cn1nnc2cc(Oc3ccc([N+](=O)[O-])cc3)ccc21 ; Cn1nnc2cc(Oc3ccc(N)cc3)ccc21]

To a solution of 1-methyl-5-(4-nitrophenoxy)-1,2,3-benzotriazole (700.0 mg, crude) in MeOH (20.0 mL) and H₂O (4.0 mL) was added Fe (723.3 mg, 12.95 mmol) and NH₄Cl (692.8 mg, 12.95 mmol) at room temperature. The resulting mixture was stirred at 60° C. for 16 h. After the reaction was completed, the resulting mixture was filtered. The filtrate was concentrated under reduced pressure. The residue was purified by flash column chromatography with petroleum ether/ethyl acetate (3/1, v/v) to afford 4-[(1-methyl-1,2,3-benzotriazol-5-yl)oxy]aniline (420.0 mg, 67%) as a yellow solid. LCMS (ESI, m/z): [M+H]⁺=241.1.

### Step 3. Synthesis of 6-chloro-N-{4-[(1-methyl-1,2,3-benzotriazol-5-yl)oxy]phenyl}pyrido[3,4-d]pyrimidin-4-amine

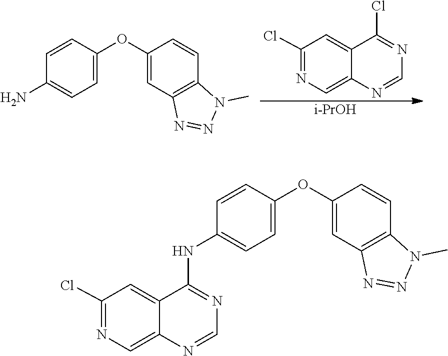 [SMILES: "i-PrOH" ; Cn1nnc2cc(Oc3ccc(N)cc3)ccc21 ; Clc1cc2c(Cl)ncnc2cn1 ; Cn1nnc2cc(Oc3ccc(Nc4ncnc5cnc(Cl)cc45)cc3)ccc21]

To a solution of 4-[(1-methyl-1,2,3-benzotriazol-5-yl)oxy]aniline (200.0 mg, 0.83 mmol) in isopropyl alcohol (10.0 mL) was added 4,6-dichloropyrido[3,4-d]pyrimidine (333.0 mg, crude) at room temperature. The resulting mixture was stirred at room temperature for 1 h. After the reaction was completed, the mixture was concentrated under vacuum. The residue was purified by flash column chromatography with CH₂Cl₂/MeOH (20/1, v/v) to afford 6-chloro-N-{4-[(1-methyl-1,2,3-benzotriazol-5-yl)oxy]phenyl}pyrido[3,4-d]pyrimidin-4-amine (110.0 mg, 33%) as a yellow solid. LCMS (ESI, m/z): [M+H]⁺=404.1.

### Step 4. Synthesis of (3E)-3-[2-(dimethylamino)ethylidene]-4-methyl-1-[4-({4-[(1-methyl-1,2,3-benzotriazol-5-yl)oxy]phenyl}amino)pyrido[3,4-d]pyrimidin-6-yl]pyrrolidin-2-one

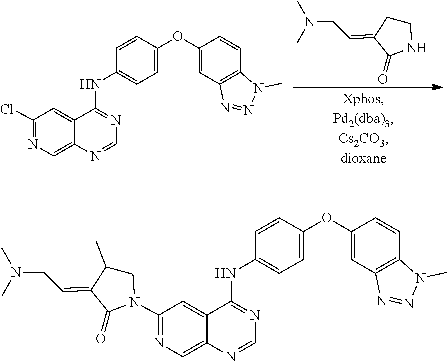

To a solution of 6-chloro-N-{4-[(1-methyl-1,2,3-benzotriazol-5-yl)oxy]phenyl}pyrido[3,4-d]pyrimidin-4-amine (100.0 mg, 0.25 mmol) in dioxane (5.0 mL) was added (3E)-3-[2-(dimethylamino)ethylidene]-4-methylpyrrolidin-2-one (41.7 mg, 0.25 mmol), Cs₂CO₃ (403.4 mg, 1.24 mmol), XPhos (23.6 mg, 0.05 mmol) and Pd₂(dba)₃ (22.7 mg, 0.03 mmol) at room temperature. The resulting mixture was stirred at 100° C. for 1 h under N₂. After the reaction was completed, the resulting mixture was filtered. The filtrate was concentrated under vacuum. The residue was purified by flash column chromatography with CH₂Cl₂/MeOH (10/1, v/v) to afford (3E)-3-[2-(dimethylamino)ethylidene]-4-methyl-1-[4-({4-[(1-methyl-1,2,3-benzotriazol-5-yl)oxy]phenyl}amino)pyrido[3,4-d]pyrimidin-6-yl]pyrrolidin-2-one (50.0 mg, 38%) as a yellow solid. LCMS (ESI, m/z): [M+H]⁺=536.2.

### Step 5. Synthesis of (3E,4R)-3-[2-(dimethylamino)ethylidene]-4-methyl-1-[4-({4-[(1-methyl-1,2,3-benzotriazol-5-yl)oxy]phenyl}amino)pyrido[3,4-d]pyrimidin-6-yl]pyrrolidin-2-one and (3E,4S)-3-[2-(dimethylamino)ethylidene]-4-methyl-1-[4-({4-[(1-methyl-1,2,3-benzotriazol-5-yl)oxy]phenyl}amino)pyrido[3,4-d]pyrimidin-6-yl]pyrrolidin-2-one (Compound 136 and Compound 137)

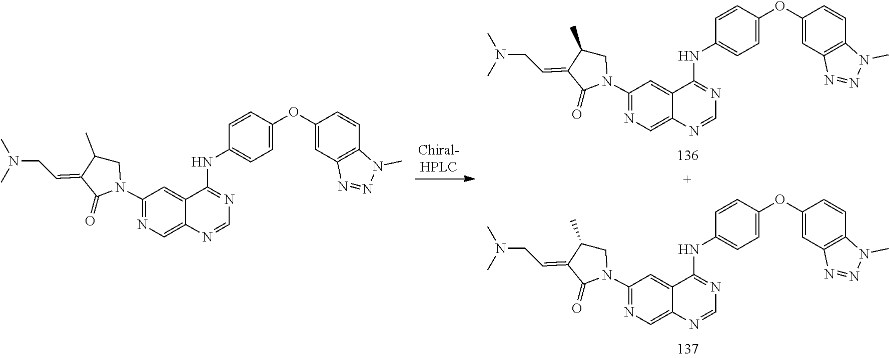

The racemic mixture of (3E)-3-[2-(dimethylamino)ethylidene]-4-methyl-1-[4-({4-[(1-methyl-1,2,3-benzotriazol-5-yl)oxy]phenyl}amino)pyrido[3,4-d]pyrimidin-6-yl]pyrrolidin-2-one (50.0 mg, 0.09 mmol) was separated by Prep-Chiral-HPLC with the following conditions (Column: CHIRALPAK IG, 2×25 cm, 5 μm; Mobile Phase A: Hex (0.5% 2M NH₃-MeOH)-HPLC, Mobile Phase B: EtOH: DCM=1: 1-HPLC; Flow rate: 20 mL/min; Gradient: 70% B to 70% B in 9.5 min; Wave Length: 220/254 nm; RT1(min): 6.87; RT2(min): 8.58) to afford (3E)-3-[2-(dimethylamino)ethylidene]-4-methyl-1-[4-({4-[(1-methyl-1,2,3-benzotriazol-5-yl)oxy]phenyl}amino)pyrido[3,4-d]pyrimidin-6-yl]pyrrolidin-2-one Enantiomer 1 (RT1 6.87 min, 13.6 mg, 54%) as a light yellow solid and (3E)-3-[2-(dimethylamino)ethylidene]-4-methyl-1-[4-({4-[(1-methyl-1,2,3-benzotriazol-5-yl)oxy]phenyl}amino)pyrido[3,4-d]pyrimidin-6-yl]pyrrolidin-2-one Enantiomer 2 (RT2 8.58 min, 16.3 mg, 64%) as a light yellow solid. The absolute stereochemistry of Enantiomers 1 and 2 was not assigned. The two enantiomeric structures that could be obtained from chiral separation of the enantiomeric mixture as described above are shown as Compounds 136 and 137 in Table 1.

(3E)-3-[2-(dimethylamino)ethylidene]-4-methyl-1-[4-({4-[(1-methyl-1,2,3-benzotriazol-5-yl)oxy]phenyl}amino)pyrido[3,4-d]pyrimidin-6-yl]pyrrolidin-2-one Enantiomer 1: RT1(min): 6.87; LCMS (ESI, m/z): [M+H]⁺=536.3. ¹H NMR (400 MHz, DMSO-d₆): δ 10.31 (s, 1H), 9.18 (s, 1H), 9.06 (s, 1H), 8.60 (s, 1H), 7.93-7.83 (m, 3H), 7.57 (s, 1H), 7.41-7.38 (m, 1H), 7.12 (d, J=8.8 Hz, 2H), 6.53-6.50 (m, 1H), 4.33 (s, 3H), 4.27-4.15 (m, 1H), 3.81-3.79 (m, 1H), 3.32-3.21 (m, 2H), 3.16-3.10 (m, 1H), 2.21 (s, 6H), 1.23 (d, J=6.8 Hz, 3H).

(3E)-3-[2-(dimethylamino)ethylidene]-4-methyl-1-[4-({4-[(1-methyl-1,2,3-benzotriazol-5-yl)oxy]phenyl}amino)pyrido[3,4-d]pyrimidin-6-yl]pyrrolidin-2-one Enantiomer 2: RT2(min): 8.58; LCMS (ESI, m/z): [M+H]⁺=536.3. ¹H NMR (400 MHz, DMSO-d₆): δ 10.31 (s, 1H), 9.18 (s, 1H), 9.07 (s, 1H), 8.60 (s, 1H), 7.92 (d, J=9.2 Hz, 1H), 7.85 (d, J=8.4 Hz, 2H), 7.57 (s, 1H), 7.45-7.38 (m, 1H), 7.11 (d, J=8.4 Hz, 2H), 6.52-6.48 (m, 1H), 4.33 (s, 3H), 4.27-4.22 (m, 1H), 3.82-3.79 (m, 1H), 3.33-3.22 (m, 2H), 3.16-3.10 (m, 1H), 2.22 (s, 6H), 1.24 (d, J=6.0 Hz, 3H).

### Example S119: Synthesis of (3E)-1-{4-[(2 fluoro-5-methyl-4-{[1,2,4]triazolo[1,5-a]pyridin-7-yloxy}phenyl)amino]pyrido[3,4-d]pyrimidin-6-yl}-3-[(1-methylazetidin-3-yl)methylidene]pyrrolidin-2-one (Compound 138)

### Step 1. Synthesis of 6-chloro-N-(2-fluoro-5-methyl-4-{[1,2,4]triazolo[1,5-a]pyridin-7-yloxy}phenyl)pyrido[3,4-d]pyrimidin-4-amine

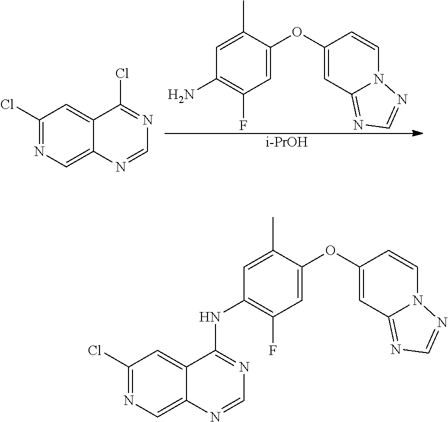 [SMILES: "i-PrOH" ; Clc1cc2c(Cl)ncnc2cn1 ; Cc1cc(N)c(F)cc1Oc1ccn2ncnc2c1 ; Cc1cc(Nc2ncnc3cnc(Cl)cc23)c(F)cc1Oc1ccn2ncnc2c1]

To a mixture of 4,6-dichloropyrido[3,4-d]pyrimidine (1.1 g, crude) in i-PrOH (40.0 mL) was added 2-fluoro-5-methyl-4-{[1,2,4]triazolo[1,5-a]pyridin-7-yloxy}aniline (1.1 g, 4.12 mmol) at 0° C. The resulting mixture was stirred at room temperature for overnight. After the reaction was completed, the mixture was concentrated under reduced pressure. The residue was purified by flash column chromatography with CH₂Cl₂/MeOH (10/1, v/v) to afford 6-chloro-N-(2-fluoro-5-methyl-4-{[1,2,4]triazolo[1,5-a]pyridin-7-yloxy}phenyl)pyrido[3,4-d]pyrimidin-4-amine (1.7 g, 73%) as a yellow solid. LCMS (ESI, m/z): [M+H]⁺=422.1.

### Step 2. Synthesis of (3E)-1-{4-[(2-fluoro-5-methyl-4-{[1,2,4]triazolo[1,5-a]pyridin-7-yloxy}phenyl)amino]pyrido[3,4-d]pyrimidin-6-yl}-3-[(1-methylazetidin-3-yl)methylidene]pyrrolidin-2-one (Compound 138)

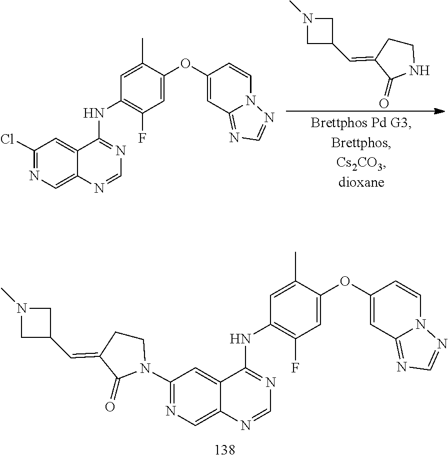 [SMILES: Cc1cc(Nc2ncnc3cnc(Cl)cc23)c(F)cc1Oc1ccn2ncnc2c1 ; CN1CC(/C=C2\CCNC2=O)C1 ; Cc1cc(Nc2ncnc3cnc(N4CC/C(=C\C5CN(C)C5)C4=O)cc23)c(F)cc1Oc1ccn2ncnc2c1]

To a mixture of 6-chloro-N-(2-fluoro-5-methyl-4-{[1,2,4]triazolo[1,5-a]pyridin-7-yloxy}phenyl)pyrido[3,4-d]pyrimidin-4-amine (200.0 mg, 0.47 mmol) in dioxane (25.0 mL) were added (3E)-3-[(1-methylazetidin-3-yl)methylidene]pyrrolidin-2-one (157.6 mg, 0.95 mmol), Cs₂CO₃ (463.5 mg, 1.42 mmol), BrettPhos (101.8 mg, 0.19 mmol) and BrettPhos Pd G3 (86.0 mg, 0.10 mmol) at room temperature under N₂. The mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (5/1, v/v) to afford (3E)-1-{4-[(2-fluoro-5-methyl-4-{[1,2,4]triazolo[1,5-a]pyridin-7-yloxy}phenyl)amino]pyrido[3,4-d]pyrimidin-6-yl}-3-[(1-methylazetidin-3-yl)methylidene]pyrrolidin-2-one (Compound 138) (14.3 mg, 5%) as a yellow solid. LCMS (ESI, m/z): [M+H]⁺=552.1. ¹H NMR (400 MHz, DMSO-d₆): δ 10.44 (s, 1H), 9.18 (s, 1H), 9.08 (s, 1H), 8.98 (d, J=7.6 Hz, 1H), 8.56 (s, 1H), 8.43 (s, 1H), 7.55 (s, 1H), 7.30 (d, J=10.8 Hz, 1H), 7.10-7.07 (m, 1H), 6.98 (d, J=2.8 Hz, 1H), 6.72-6.68 (m, 1H), 4.15-4.12 (m, 2H), 3.53-3.49 (m, 2H), 3.30-3.24 (m, 1H), 2.98-2.95 (m, 2H), 2.82-2.79 (m, 2H), 2.23 (s, 3H), 2.19 (s, 3H).

### Example S120: Synthesis of (3E)-1-{4-[(3-methyl-4-{[1,2,4]triazolo[1,5-a]pyridin-7-yloxy}phenyl)amino]pyrido[3,2-d]pyrimidin-6-yl}-3-[(1-methylazetidin-3-yl)methylidene]pyrrolidin-2-one (Compound 139)

### Step 1. Synthesis of Tert-butyl 3-{[1-(tert-butoxycarbonyl)azetidin-3-yl](hydroxy)methyl}-2-oxopyrrolidine-1-carboxylate

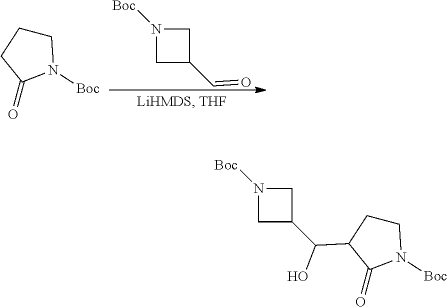 [SMILES: CC(C)(C)OC(=O)N1CCCC1=O ; CC(C)(C)OC(=O)N1CC(C=O)C1 ; CC(C)(C)OC(=O)N1CC(C(O)C2CCN(C(=O)OC(C)(C)C)C2=O)C1]

To a solution of tert-butyl 2-oxopyrrolidine-1-carboxylate (10.0 g, 53.99 mmol) in THF (200.0 mL) was added LiHMDS (90.0 mL, 1 mol/L) at −78° C. under N₂. The mixture was stirred at −78° C. for 2 h under N₂. Then a solution of tert-butyl 3-formylazetidine-1-carboxylate (12.0 g, 12.96 mmol) in THF (40.0 mL) was added dropwise to the mixture at −78° C. The mixture was stirred at −78° C. for additional 2 h. After the reaction was completed, the reaction mixture was quenched with NH₄Cl (sat.) and then extracted with ethyl acetate. The combined organic layer was washed with brine, dried over anhydrous sodium sulfate and filtered. The filtrate was concentrated under vacuum to afford tert-butyl 3-{[1-(tert-butoxycarbonyl)azetidin-3-yl](hydroxy)methyl}-2-oxopyrrolidine-1-carboxylate (19.0 g, crude) as a yellow oil. LCMS (ESI, m/z): [M+H]⁺=371.2.

### Step 2. Synthesis of Tert-butyl 3-{[1-(tert-butoxycarbonyl)azetidin-3-yl](methanesulfonyloxy)methyl}-2-oxopyrrolidine-1-carboxylate

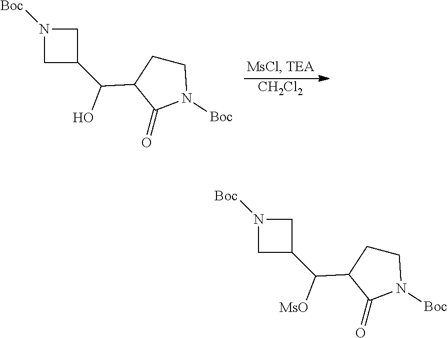

To a mixture of tert-butyl 3-{[1-(tert-butoxycarbonyl)azetidin-3-yl](hydroxy)methyl}-2-oxopyrrolidine-1-carboxylate (19.0 g, crude) in CH₂Cl₂ (400.0 mL) was added TEA (10.4 g, 102.58 mmol) at room temperature. Then MsCl (10.0 g, 87.19 mmol) was added to the mixture at 0° C. The mixture was stirred at room temperature for 16 h. After the reaction was completed, the mixture was diluted with H₂O and extracted with CH₂Cl₂. The combined organic layer was washed with brine, dried over anhydrous sodium sulfate and filtered. The filtrate was concentrated under vacuum to afford tert-butyl 3-{[1-(tert-butoxycarbonyl)azetidin-3-yl](methanesulfonyloxy)methyl}-2-oxopyrrolidine-1-carboxylate (20.0 g, crude) as a brown oil. LCMS (ESI, m/z): [M+H]⁺=449.2.

### Step 3. Synthesis of Tert-butyl(3E)-3-{[1-(tert-butoxycarbonyl)azetidin-3-yl]methylidene}-2-oxopyrrolidine-1-carboxylate DBU, CH₂Cl₁₋₂

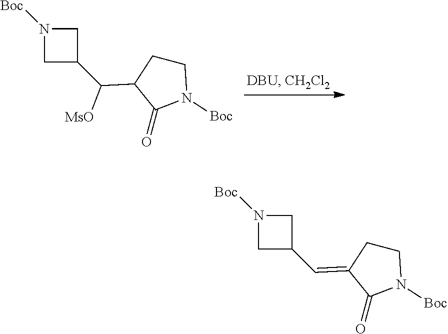 [SMILES: CC(C)(C)OC(=O)N1CC(C(OS(C)(=O)=O)C2CCN(C(=O)OC(C)(C)C)C2=O)C1 ; CC(C)(C)OC(=O)N1CC(/C=C2\CCN(C(=O)OC(C)(C)C)C2=O)C1]

To a solution of tert-butyl 3-{[1-(tert-butoxycarbonyl)azetidin-3-yl](methanesulfonyloxy)methyl}-2-oxopyrrolidine-1-carboxylate (20.0 g, crude) in CH₂Cl₂ (400.0 mL) was added DBU (17.0 g, 111.48 mmol) at 0° C. The mixture was stirred at room temperature for 3 h. After the reaction was completed, the mixture was diluted with H₂O and extracted with CH₂Cl₂. The combined organic layer was washed with brine, dried over anhydrous sodium sulfate and filtered. The filtrate was evaporated in vacuo. The residue was purified by flash column chromatography with petroleum ether/EtOAc (2/1, v/v) to afford tert-butyl (3E)-3-{[1-(tert-butoxycarbonyl)azetidin-3-yl]methylidene}-2-oxopyrrolidine-1-carboxylate (6.7 g, 42%) as a yellow solid. LCMS (ESI, m/z): [M+H]⁺=353.2.

### Step 4. Synthesis of (E)-3-(azetidin-3-ylmethylene)pyrrolidin-2-one 2,2,2-trifluoroacetate

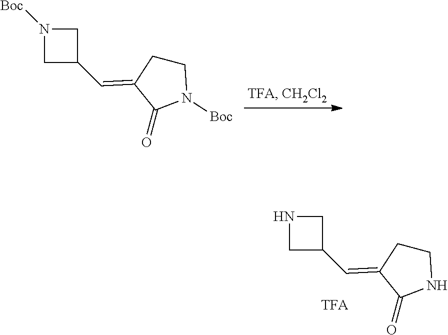 [SMILES: CC(C)(C)OC(=O)N1CC(/C=C2\CCN(C(=O)OC(C)(C)C)C2=O)C1 ; O=C1NCC/C1=C\C1CNC1]

To a solution of tert-butyl (3E)-3-{[1-(tert-butoxycarbonyl)azetidin-3-yl]methylidene}-2-oxopyrrolidine-1-carboxylate (6.7 g, 19.01 mmol) in CH₂Cl₂ (40.0 mL) was added TFA (20.0 mL) at room temperature. The mixture was stirred at room temperature for 1 h. After the reaction was completed, the mixture was concentrated under reduced pressure to afford (E)-3-(azetidin-3-ylmethylene)pyrrolidin-2-one 2,2,2-trifluoroacetate (10.0 g, crude) as a brown oil. LCMS (ESI, m/z): [M+H]⁺=153.1.

### Step 5. Synthesis of (3E)-3-[(1-methylazetidin-3-yl)methylidene]pyrrolidin-2-one

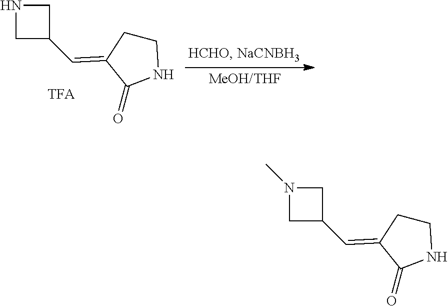

To a mixture of (E)-3-(azetidin-3-ylmethylene)pyrrolidin-2-one 2,2,2-trifluoroacetate (10.0 g, crude) in MeOH (75.0 mL)/THF (375.0 mL) was added HCHO (8.4 g, 40% in H₂O) at room temperature. The mixture was stirred at room temperature for 1 h. Then NaBH₃CN (11.3 g, 179.84 mmol) was added to the mixture at 0° C. The mixture was stirred at room temperature for additional 2 h. After the reaction was completed, the resulting mixture was concentrated under reduced pressure. The residue was purified by reverse phase flash chromatography with H₂O/ACN (95/5, v/v) afford (3E)-3-[(1-methylazetidin-3-yl)methylidene]pyrrolidin-2-one (1.0 g, 15%) as a colorless oil. LCMS (ESI, m/z): [M+H]⁺=167.1.

### Step 6. Synthesis of (3E)-1-{4-[(3-methyl-4-{[1,2,4]triazolo[1,5-a]pyridin-7-yloxy}phenyl)amino]pyrido[3,2-d]pyrimidin-6-yl}-3-[(1-methylazetidin-3-yl)methylidene]pyrrolidin-2-one (Compound 139)

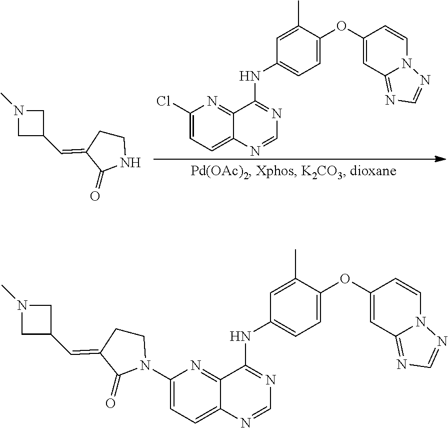 [SMILES: CN1CC(/C=C2\CCNC2=O)C1 ; Cc1cc(Nc2ncnc3ccc(Cl)nc23)ccc1Oc1ccn2ncnc2c1 ; Cc1cc(Nc2ncnc3ccc(N4CC/C(=C\C5CN(C)C5)C4=O)nc23)ccc1Oc1ccn2ncnc2c1]

To a mixture of 6-chloro-N-(3-methyl-4-{[1,2,4]triazolo[1,5-a]pyridin-7-yloxy}phenyl)pyrido[3,2-d]pyrimidin-4-amine (242.9 mg, 0.60 mmol) in dioxane (20.0 mL) were added (3E)-3-[(1-methylazetidin-3-yl)methylidene]pyrrolidin-2-one (200.0 mg, 1.20 mmol), K₂CO₃ (415.7 mg, 3.01 mmol), XPhos (57.4 mg, 0.12 mmol) and Pd(OAc)₂ (13.5 mg, 0.06 mmol) at room temperature under N₂. The mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (5/1, v/v) to afford (3E)-1-{4-[(3-methyl-4-{[1,2,4]triazolo[1,5-a]pyridin-7-yloxy}phenyl)amino]pyrido[3,2-d]pyrimidin-6-yl}-3-[(1-methylazetidin-3-yl)methylidene]pyrrolidin-2-one (Compound 139) (107.9 mg, 33%) as a light yellow solid. LCMS (ESI, m/z): [M+H]⁺=534.2. ¹H NMR (400 MHz, DMSO-d₆): δ 9.57 (s, 1H), 8.99-8.94 (m, 2H), 8.63 (s, 1H), 8.39 (s, 1H), 8.26 (d, J=9.2 Hz, 1H), 7.99-7.95 (m, 2H), 7.25 (d, J=8.4 Hz, 1H), 7.05-7.03 (m, 1H), 6.81-6.74 (m, 2H), 4.33-4.30 (m, 2H), 3.60-3.56 (m, 2H), 3.37-3.32 (m, 1H), 3.10-3.06 (m, 2H), 2.84-2.81 (m, 2H), 2.29 (s, 3H), 2.22 (s, 3H).

### Example S121: Synthesis of (3E)-1-{4-[(2,5-difluoro-4-{[1,2,4]triazolo[1,5-a]pyridin-7-yloxy}phenyl)amino]pyrido[3,4-d]pyrimidin-6-yl}-3-[2-(dimethylamino)ethylidene]pyrrolidin-2-one (Compound 140)

### Step 1. Synthesis of 4,6-dichloropyrido[3,4-d]pyrimidine

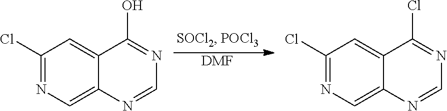

To a solution of 6-chloropyrido[3,4-d]pyrimidin-4-ol (250.0 mg, 1.38 mmol) in SOCl₂ (10.0 mL) was added DMF (0.1 mL) and POCl₃ (2.0 mL) at room temperature. The resulting mixture was stirred at 85° C. for 2 h. After the reaction was completed, the mixture was concentrated under reduced pressure to afford 4,6-dichloropyrido[3,4-d]pyrimidine (200.0 mg, crude) as a yellow solid.

### Step 2. Synthesis of 6-chloro-N-(2,5-difluoro-4-{[1,2,4]triazolo[1,5-a]pyridin-7-yloxy}phenyl)pyrido[3,4-d]pyrimidin-4-amine

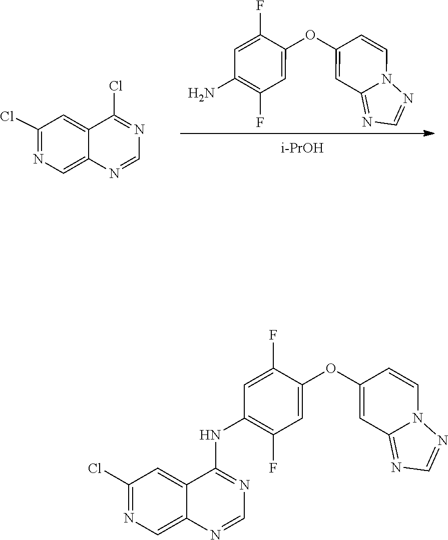 [SMILES: "i-PrOH" ; Clc1cc2c(Cl)ncnc2cn1 ; Nc1cc(F)c(Oc2ccn3ncnc3c2)cc1F ; Fc1cc(Oc2ccn3ncnc3c2)c(F)cc1Nc1ncnc2cnc(Cl)cc12]

To a solution of 2,5-difluoro-4-{[1,2,4]triazolo[1,5-a]pyridin-7-yloxy}aniline (131.1 mg, 0.50 mmol) in i-PrOH (5.0 mL) was added 4,6-dichloropyrido[3,4-d]pyrimidine (100.0 mg, crude) at room temperature. The resulting mixture was stirred at room temperature for 16 h. After the reaction was completed, the resulting mixture was concentrated under reduced pressure. The residue was purified by flash column chromatography with dichloromethane/methanol (83/17, v/v) to afford 6-chloro-N-(2,5-difluoro-4-{[1,2,4]triazolo[1,5-a]pyridin-7-yloxy}phenyl)pyrido[3,4-d]pyrimidin-4-amine (175.8 mg, 82%) as a yellow solid. LCMS (ESI, m/z): [M+H]⁺=426.1.

### Step 3. Synthesis of (3E)-1-{4-[(2,5-difluoro-4-{[1,2,4]triazolo[1,5-a]pyridin-7-yloxy}phenyl)amino]pyrido[3,4-d]pyrimidin-6-yl}-3-[2-(dimethylamino)ethylidene]pyrrolidin-2-one (Compound 140)

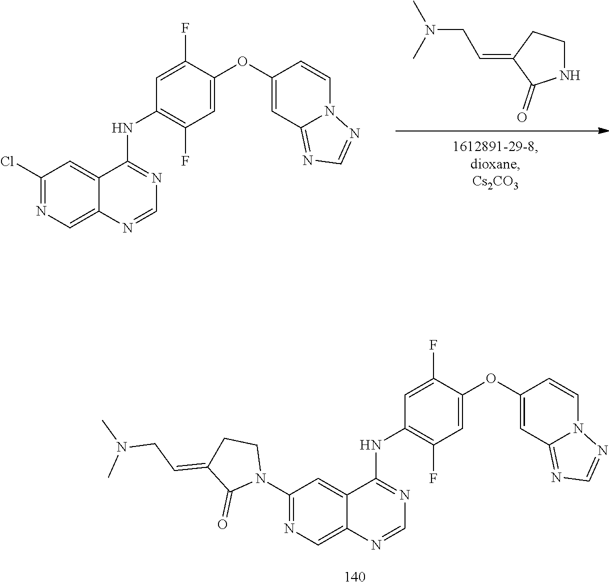 [SMILES: Fc1cc(Oc2ccn3ncnc3c2)c(F)cc1Nc1ncnc2cnc(Cl)cc12 ; CN(C)C/C=C1\CCNC1=O ; CN(C)C/C=C1\CCN(c2cc3c(Nc4cc(F)c(Oc5ccn6ncnc6c5)cc4F)ncnc3cn2)C1=O]

To a solution of 6-chloro-N-(2,5-difluoro-4-{[1,2,4]triazolo[1,5-a]pyridin-7-yloxy}phenyl)pyrido[3,4-d]pyrimidin-4-amine (200.0 mg, 0.47 mmol) in dioxane (5.0 mL) was added Cs₂CO₃ (459.1 mg, 1.41 mmol), (3E)-3-[2-(dimethylamino)ethylidene]pyrrolidin-2-one (72.3 mg, 0.47 mmol) and Pd-PEPPSI-IPentCl 2-methylpyridine (o-picoline (79.0 mg, 0.09 mmol) at room temperature under N₂. The resulting mixture was stirred at 80° C. for 16 h. After the reaction was completed, the mixture was cooled to room temperature and then filtered. The filtrate was concentrated under reduced pressure. The residue was purified by flash column chromatography with dichloromethane/methanol (90/10, v/v) and then purified by Prep-HPLC with the following conditions: (Column: XBridge Prep Phenyl OBD Column, 19×250 mm, 5 μm; Mobile Phase A: Water (10 mmol/L NH₄HCO₃), Mobile Phase B: ACN; Flow rate: 25 mL/min; Gradient: 30% B to 45% B in 10 min; Wave Length: 254 nm) to afford (3E)-1-{4-[(2,5-difluoro-4-{[1,2,4]triazolo[1,5-a]pyridin-7-yloxy}phenyl)amino]pyrido[3,4-d]pyrimidin-6-yl}-3-[2-(dimethylamino)ethylidene]pyrrolidin-2-one (Compound 140) (13.3 mg, 5%) as a white solid. LCMS (ESI, m/z): [M+H]⁺=544.2. ¹H NMR (400 MHz, CD₃OD): δ 9.14 (s, 1H), 9.07 (s, 1H), 8.82-8.80 (m, 1H), 8.55 (s, 1H), 8.36 (s, 1H), 7.88-7.84 (m, 1H), 7.45-7.41 (m, 1H), 7.17-7.15 (m, 2H), 6.74-6.71 (m, 1H), 4.31-4.28 (m, 2H), 3.24 (d, J=6.8 Hz, 2H), 2.99-2.92 (m, 2H), 2.36 (s, 6H).

### Example S122: Synthesis of (E)-1-(4-((4-([1,2,4]triazolo[1,5-a]pyridin-7-yloxy)-2,5-difluorophenyl)amino)pyrido[3,2-d]pyrimidin-6-yl)-3-(2-(dimethylamino)ethylidene)pyrrolidin-2-one (Compound 141)

### Step 1. Synthesis of 4,6-dichloropyrido[3,2-d]pyrimidine

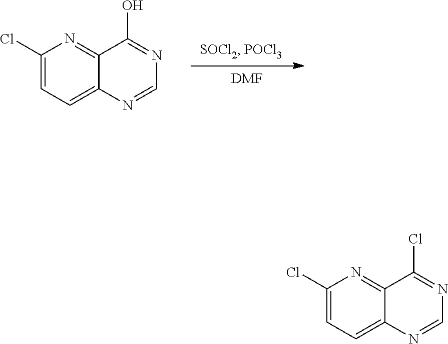

To a solution of 6-chloropyrido[3,2-d]pyrimidin-4-ol (300.0 mg, 1.65 mmol) in SOCl₂ (10.0 mL) was added POCl₃ (2.0 mL) and DMF (0.1 mL) at room temperature. The reaction mixture was stirred at 85° C. for 1 h. After the reaction was completed, the mixture was concentrated under reduced pressure to afford 4,6-dichloropyrido[3,2-d]pyrimidine (325.0 mg, crude) as a yellow solid.

### Step 2. Synthesis of N-(4-([1,2,4]triazolo[1,5-a]pyridin-7-yloxy)-2,5-difluorophenyl)-6-chloropyrido[3,2-d]pyrimidin-4-amine

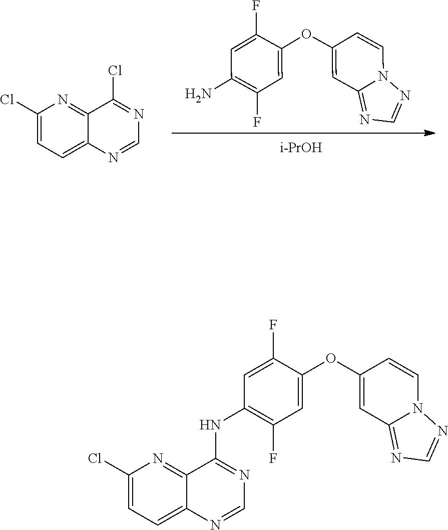 [SMILES: "i-PrOH" ; Clc1ccc2ncnc(Cl)c2n1 ; Nc1cc(F)c(Oc2ccn3ncnc3c2)cc1F ; Fc1cc(Oc2ccn3ncnc3c2)c(F)cc1Nc1ncnc2ccc(Cl)nc12]

To a solution of 4-([1,2,4]triazolo[1,5-a]pyridin-7-yloxy)-2,5-difluoroaniline (426.1 mg, 1.63 mmol) in isopropyl alcohol (15.0 mL) was added 4,6-dichloropyrido[3,2-d]pyrimidine (325.0 mg, crude) at room temperature. The resulting mixture was stirred at room temperature for 3 h. After the reaction was completed, the resulting mixture was concentrated under reduced pressure. The residue was purified by flash column chromatography with dichloromethane/methanol (85/15, v/v) to afford N-(4-([1,2,4]triazolo[1,5-a]pyridin-7-yloxy)-2,5-difluorophenyl)-6-chloropyrido[3,2-d]pyrimidin-4-amine (550.0 mg, 79%) as a yellow solid. LCMS (ESI, m/z): [M+H]⁺=426.1.

### Step 3. Synthesis of (E)-1-(4-((4-([1,2,4]triazolo[1,5-a]pyridin-7-yloxy)-2,5-difluorophenyl)amino)pyrido[3,2-d]pyrimidin-6-yl)-3-(2-(dimethylamino)ethylidene)pyrrolidin-2-one (Compound 141)

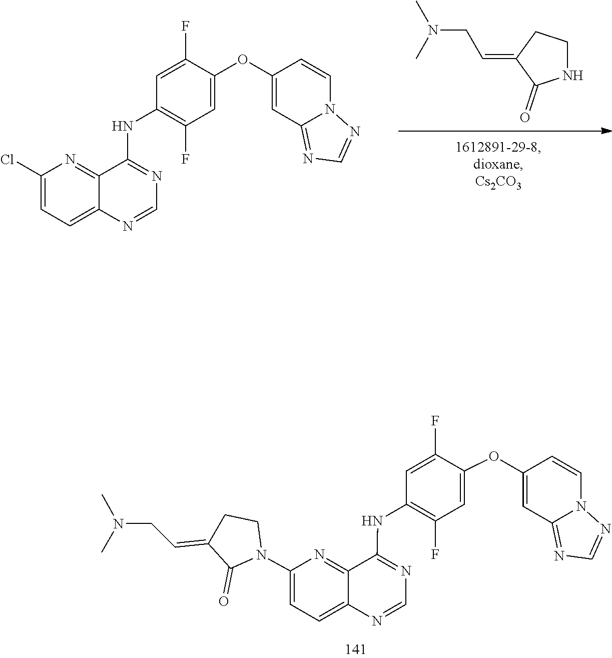 [SMILES: Fc1cc(Oc2ccn3ncnc3c2)c(F)cc1Nc1ncnc2ccc(Cl)nc12 ; CN(C)C/C=C1\CCNC1=O ; CN(C)C/C=C1\CCN(c2ccc3ncnc(Nc4cc(F)c(Oc5ccn6ncnc6c5)cc4F)c3n2)C1=O]

To a solution of N-(4-([1,2,4]triazolo[1,5-a]pyridin-7-yloxy)-2,5-difluorophenyl)-6-chloropyrido[3,2-d]pyrimidin-4-amine (100.0 mg, 0.23 mmol) in 1,4-dioxane (10.0 mL) was added (E)-3-(2-(dimethylamino)ethylidene)pyrrolidin-2-one (39.8 mg, 0.25 mmol), Cs₂CO₃ (53.6 mg, 0.70 mmol) and Pd-PEPPSI-IPentCl 2-methylpyridine (o-picoline) (39.5 mg, 0.04 mmol) at room temperature under N₂. The resulting mixture was stirred at 100° C. for 16 h under N₂. After the reaction was completed, the mixture was filtered. The filtrate was concentrated under reduced pressure. The residue was purified by flash column chromatography with CH₂Cl₂/MeOH (90/10, v/v) and then purified by Prep-HPLC with the following conditions: (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 32% B to 40% B in 8 min; Wave Length: 254 nm) to afford (E)-1-(4-((4-([1,2,4]triazolo[1,5-a]pyridin-7-yloxy)-2,5-difluorophenyl)amino)pyrido[3,2-d]pyrimidin-6-yl)-3-(2-(dimethylamino)ethylidene)pyrrolidin-2-one (Compound 141) (9.6 mg, 7%) as a light yellow solid. LCMS (ESI, m/z): [M+H]⁺=544.3. ¹H NMR (400 MHz, DMSO-d₆): δ 9.57 (s, 1H), 9.04-9.00 (m, 2H), 8.69 (s, 1H), 8.45 (s, 1H), 8.39-8.32 (m, 2H), 7.72-7.67 (m, 1H), 7.20 (d, J=2.4 Hz, 1H), 7.16-7.14 (m, 1H), 6.60-6.57 (m, 1H), 4.29-4.25 (m, 2H), 3.12 (d, J=6.4 Hz, 2H), 2.93-2.86 (m, 2H), 2.22 (s, 6H).

### Example S123: Synthesis of (4Z)-4 [2-(dimethylamino)ethylidene]-2-{4-[(3-methyl-4-{[1,2,4]triazolo[1,5-a]pyridin-7-yloxy}phenyl)amino]pyrido[3,2-d]pyrimidin-6-yl}-2-azabicyclo[3.1.0]hexan-3-one (Compound 142)

### Step 1. Synthesis of Tert-butyl (4Z)-4-(2-hydroxyethylidene)-3-oxo-2-azabicyclo[3.1.0]hexane-2-carboxylate

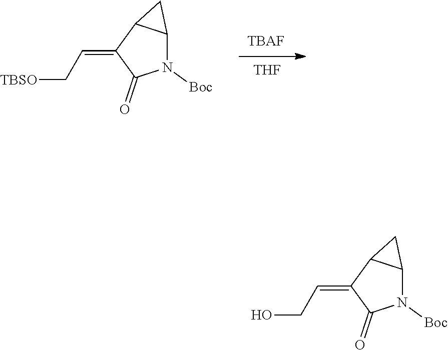 [SMILES: CC(C)(C)OC(=O)N1C(=O)/C(=C\CO[Si](C)(C)C(C)(C)C)C2CC21 ; "TBAF" ; CC(C)(C)OC(=O)N1C(=O)/C(=C\CO)C2CC21 ; "THF"]

To a mixture of tert-butyl (4Z)-4-{2-[(tert-butyldiphenylsilyl)oxy]ethylidene}-3-oxo-2-azabicyclo[3.1.0]hexane-2-carboxylate (300.0 mg, 0.62 mmol) in THE (5.0 mL) was added TBAF (0.6 mL, 1 mol/L) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (1/1, v/v) to afford tert-butyl (4Z)-4-(2-hydroxyethylidene)-3-oxo-2-azabicyclo[3.1.0]hexane-2-carboxylate (200.0 mg, 93%) as a white solid. LCMS (ESI, m/z): [M+H]⁺=240.1.

### Step 2. Synthesis of Tert-butyl (4Z)-4-[2-(methanesulfonyloxy)ethylidene]-3-oxo-2-azabicyclo[3.1.0]hexane-2-carboxylate

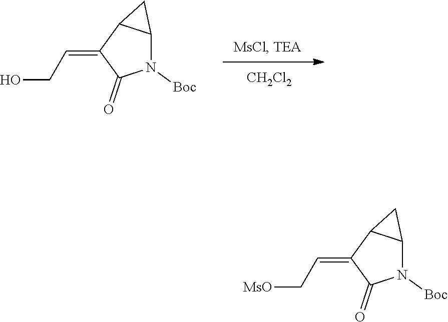 [SMILES: CC(C)(C)OC(=O)N1C(=O)/C(=C\CO)C2CC21 ; "CH2Cl2" ; CC(C)(C)OC(=O)N1C(=O)/C(=C\COS(C)(=O)=O)C2CC21]

To a solution of tert-butyl (4Z)-4-(2-hydroxyethylidene)-3-oxo-2-azabicyclo[3.1.0]hexane-2-carboxylate (200.0 mg, 0.83 mmol) in CH₂Cl₂ (20.0 mL) was added TEA (253.7 mg, 2.50 mmol) and MsCl (191.4 mg, 1.67 mmol) at 0° C. The resulting mixture was stirred at room temperature for 2 h. After the reaction was completed, the reaction mixture was diluted with H₂O and extracted with CH₂Cl₂. The combined organic layer was washed with brine, dried over anhydrous sodium sulfate and filtered. The filtrate was concentrated under vacuum to afford tert-butyl (4Z)-4-[2-(methanesulfonyloxy)ethylidene]-3-oxo-2-azabicyclo[3.1.0]hexane-2-carboxylate (200.0 mg, crude) as a yellow oil. LCMS (ESI, m/z): [M+H]⁺=318.1.

### Step 3. Synthesis of Tert-butyl (4Z)-4-[2-(dimethylamino)ethylidene]-3-oxo-2-azabicyclo[3.1.0]hexane-2-carboxylate

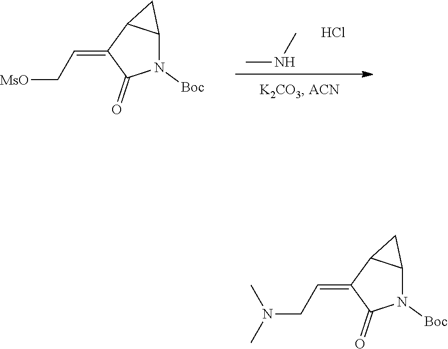

To a solution of tert-butyl (4Z)-4-[2-(methanesulfonyloxy)ethylidene]-3-oxo-2-azabicyclo[3.1.0]hexane-2-carboxylate (200.0 mg, crude) in ACN (5.0 mL) were added dimethylamine hydrochloride (51.3 mg, 0.63 mmol) and K₂CO₃ (261.2 mg, 1.89 mmol) at room temperature. The mixture was stirred at 60° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford tert-butyl (4Z)-4-[2-(dimethylamino)ethylidene]-3-oxo-2-azabicyclo[3.1.0]hexane-2-carboxylate (50.0 mg, 29%) as a yellow oil. LCMS (ESI, m/z): [M+H]⁺=267.2.

### Step 4. Synthesis of (Z)-4-(2-(dimethylamino)ethylidene)-2-azabicyclo[3.1.0]hexan-3-one 2,2,2-trifluoroacetate

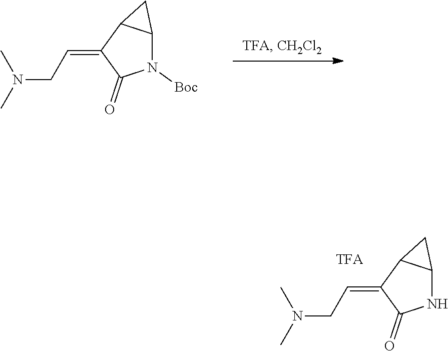 [SMILES: CN(C)C/C=C1\C(=O)N(C(=O)OC(C)(C)C)C2CC12 ; CN(C)C/C=C1\C(=O)NC2CC12]

To a solution of tert-butyl (4Z)-4-[2-(dimethylamino)ethylidene]-3-oxo-2-azabicyclo[3.1.0]hexane-2-carboxylate (50.0 mg, 0.18 mmol) in CH₂Cl₂ (1.0 mL) were added TFA (0.5 mL) at room temperature. The mixture was stirred at room temperature for 1 h. After the reaction was completed, the reaction mixture was concentrated under reduced pressure to afford (Z)-4-(2-(dimethylamino)ethylidene)-2-azabicyclo[3.1.0]hexan-3-one 2,2,2-trifluoroacetate (50.0 mg, crude) as a light yellow oil. LCMS (ESI, m/z): [M+H]⁺=167.1.

### Step 5. Synthesis of (4Z)-4-[2-(dimethylamino)ethylidene]-2-{4-[(3-methyl-4-{[1,2,4]triazolo[1,5-a]pyridin-7-yloxy}phenyl)amino]pyrido[3,2-d]pyrimidin-6-yl}-2-azabicyclo[3.1.0]hexan-3-one (Compound 142)

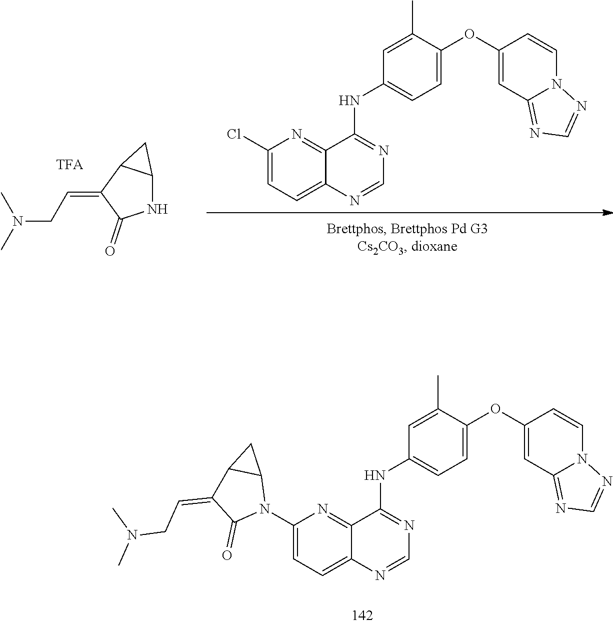 [SMILES: CN(C)C/C=C1\C(=O)NC2CC12 ; Cc1cc(Nc2ncnc3ccc(Cl)nc23)ccc1Oc1ccn2ncnc2c1 ; Cc1cc(Nc2ncnc3ccc(N4C(=O)/C(=C\CN(C)C)C5CC54)nc23)ccc1Oc1ccn2ncnc2c1]

To a solution of (Z)-4-(2-(dimethylamino)ethylidene)-2-azabicyclo[3.1.0]hexan-3-one 2,2,2-trifluoroacetate (50.0 mg, crude) in 1,4-dioxane (2.0 mL) was added 6-chloro-N-(3-methyl-4-{[1,2,4]triazolo[1,5-a]pyridin-7-yloxy}phenyl)pyrido[3,2-d]pyrimidin-4-amine (121.4 mg, 0.30 mmol), Cs₂CO₃ (68.7 mg, 0.90 mmol), BrettPhos (32.2 mg, 0.06 mmol) and BrettPhos Pd G3 (27.2 mg, 0.03 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 35% B to 45% B in 8 min; Wave Length: 254 nm) to afford (4Z)-4-[2-(dimethylamino)ethylidene]-2-{4-[(3-methyl-4-{[1,2,4]triazolo[1,5-a]pyridin-7-yloxy}phenyl)amino]pyrido[3,2-d]pyrimidin-6-yl}-2-azabicyclo[3.1.0]hexan-3-one (Compound 142) (8.9 mg, 5%) as a yellow solid. LCMS (ESI, m/z): [M+H]⁺=534.1. ¹H NMR (400 MHz, DMSO-d₆): δ 9.70 (s, 1H), 8.94 (d, J=7.6 Hz, 1H), 8.87 (d, J=9.2 Hz, 1H), 8.64 (s, 1H), 8.38 (s, 1H), 8.27 (d, J=9.2 Hz, 1H), 7.97-7.94 (m, 2H), 7.28-7.25 (m, 1H), 7.05-7.03 (m, 1H), 6.81 (d, J=2.4 Hz, 1H), 6.61-6.56 (m, 1H), 5.10-5.07 (m, 1H), 3.34-3.22 (m, 2H), 2.61-2.52 (m, 1H), 2.26-2.23 (m, 9H), 1.44-1.42 (m, 1H), 0.83-0.80 (m, 1H).

### Example S124: Synthesis of (R,E)-3-(2-(dimethylamino)ethylidene)-1-(4-((3-fluoro-2-methyl-4-((1-methyl-1H-benzo[d][1,2,3]triazol-5-yl)oxy)phenyl)amino)pyrido[3,4-d]pyrimidin-6-yl)-4-methylpyrrolidin-2-one and (S,E)-3-(2-(dimethylamino)ethylidene)-1-(4-((3-fluoro-2-methyl-4-((1-methyl-1H-benzo[d][1,2,3]triazol-5-yl)oxy)phenyl)amino)pyrido[3,4-d]pyrimidin-6-yl)-4-methylpyrrolidin-2-one (Compound 143 and Compound 144)

### Step 1. Synthesis of 5-(2-fluoro-3-methyl-4-nitrophenoxy)-1-methyl-1H-benzo[d][1,2,3]triazole

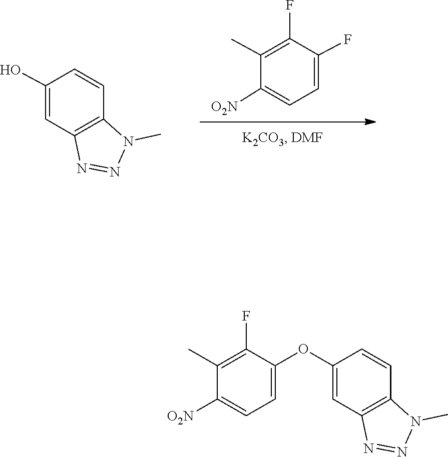

To a solution of 1-methyl-1H-benzo[d][1,2,3]triazol-5-ol (500.0 mg, 3.35 mmol) in DMF (15.0 mL) was added 1,2-difluoro-3-methyl-4-nitrobenzene (696.4 mg, 4.02 mmol) and K₂CO₃ (926.6 mg, 6.70 mmol) at room temperature. The resulting mixture was stirred at 50° C. for 16 h. After the reaction was completed, the resulting mixture was diluted with H₂O and then filtered. The solid was collected and dried to afford 5-(2-fluoro-3-methyl-4-nitrophenoxy)-1-methyl-1H-benzo[d][1,2,3]triazole (700.0 mg, crude) as a brown solid. LCMS (ESI, m/z): [M+H]⁺=303.1.

### Step 2. Synthesis of 3-fluoro-2-methyl-4-((1-methyl-1H-benzo[d][1,2,3]triazol-5-yl)oxy)aniline

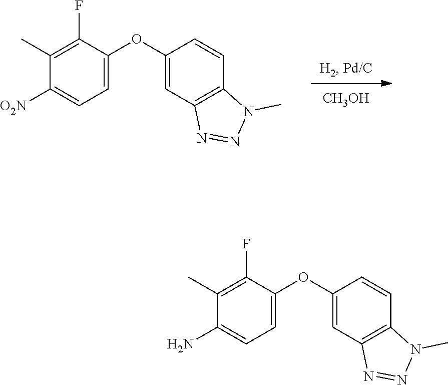

To a solution of 5-(2-fluoro-3-methyl-4-nitrophenoxy)-1-methyl-1H-benzo[d][1,2,3]triazole (700.0 mg, crude) in MeOH (20.0 mL) was added Pd/C (344.0 mg, 10%) at room temperature under N₂. The resulting mixture was stirred at room temperature for 1 h under H₂. After the reaction was completed, the resulting mixture was filtered. The filtrate was concentrated under reduced pressure. The residue was purified by flash column chromatography with petroleum ether/ethyl acetate (1/1, v/v) to afford 3-fluoro-2-methyl-4-((1-methyl-1H-benzo[d][1,2,3]triazol-5-yl)oxy)aniline (500.0 mg, 71%) as a yellow solid. LCMS (ESI, m/z): [M+H]⁺=273.1.

### Step 3. Synthesis of 6-chloro-N-(3-fluoro-2-methyl-4-((1-methyl-1H-benzo[d][1,2,3]triazol-5-yl)oxy)phenyl)pyrido[3,4-d]pyrimidin-4-amine

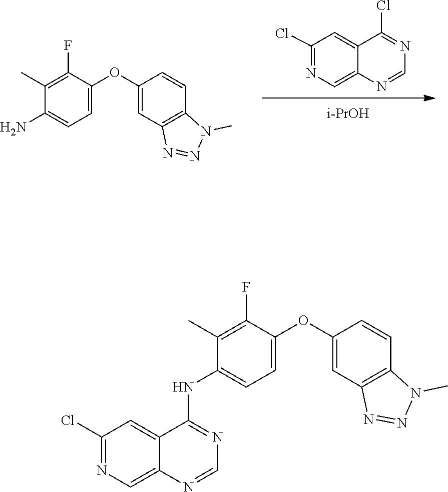

To a solution of 3-fluoro-2-methyl-4-((1-methyl-1H-benzo[d][1,2,3]triazol-5-yl)oxy)aniline (500.0 mg, 1.84 mmol) in isopropyl alcohol (10.0 mL) was added 4,6-dichloropyrido[3,4-d]pyrimidine (734.6 mg, crude) at room temperature. The resulting mixture was stirred at room temperature for 1 h. After the reaction was completed, The pH value of the mixture was adjusted to 7 with aq.NaHCO₃ and then extracted with EtOAc. The combined organic layers were washed with brine and dried over anhydrous Na₂SO₄. After filtration, the filtrate was concentrated under vacuum. The residue was purified by flash column chromatography with CH₂Cl₂/MeOH (10/1, v/v) to afford 6-chloro-N-(3-fluoro-2-methyl-4-((1-methyl-1H-benzo[d][1,2,3]triazol-5-yl)oxy)phenyl)pyrido[3,4-d]pyrimidin-4-amine (300.0 mg, 33%) as a brown solid. LCMS (ESI, m/z): [M+H]⁺=436.1.

### Step 4. Synthesis of (E)-3-(2-(dimethylamino)ethylidene)-1-(4-((3-fluoro-2-methyl-4-((1-methyl-1H-benzo[d][1,2,3]triazol-5-yl)oxy)phenyl)amino)pyrido[3,4-d]pyrimidin-6-yl)-4-methylpyrrolidin-2-one

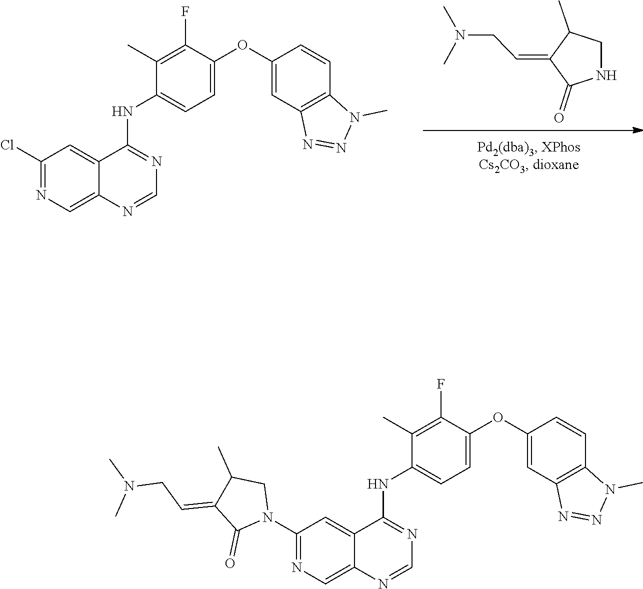

To a solution of 6-chloro-N-(3-fluoro-2-methyl-4-((1-methyl-1H-benzo[d][1,2,3]triazol-5-yl)oxy)phenyl)pyrido[3,4-d]pyrimidin-4-amine (200.0 mg, 0.46 mmol) in dioxane (6.0 mL) was added (E)-3-(2-(dimethylamino)ethylidene)-4-methylpyrrolidin-2-one (77.2 mg, 0.46 mmol), X-Phos (43.7 mg, 0.09 mmol), Cs₂CO₃ (747.6 mg, 2.30 mmol) and Pd₂(dba)₃ (26.4 mg, 0.03 mmol) at room temperature under N₂. The resulting mixture was stirred at 100° C. for 1 h. After the reaction was completed, the resulting mixture was filtered. The filtrate was concentrated under vacuum. The residue was purified by flash column chromatography with CH₂Cl₂/MeOH (9/1, v/v) to afford (E)-3-(2-(dimethylamino)ethylidene)-1-(4-((3-fluoro-2-methyl-4-((1-methyl-1H-benzo[d][1,2,3]triazol-5-yl)oxy)phenyl)amino)pyrido[3,4-d]pyrimidin-6-yl)-4-methylpyrrolidin-2-one (50.0 mg, 18%) as a brown solid. LCMS (ESI, m/z): [M+H]⁺=568.3.

### Step 5. Synthesis of (R,E)-3-(2-(dimethylamino)ethylidene)-1-(4-((3-fluoro-2-methyl-4-((1-methyl-1H-benzo[d][1,2,3]triazol-5-yl)oxy)phenyl)amino)pyrido[3,4-d]pyrimidin-6-yl)-4-methylpyrrolidin-2-one and (S,E)-3-(2-(dimethylamino)ethylidene)-1-(4-((3-fluoro-2-methyl-4-((1-methyl-1H-benzo[d][1,2,3]triazol-5-yl)oxy)phenyl)amino)pyrido[3,4-d]pyrimidin-6-yl)-4-methylpyrrolidin-2-one (Compound 143 and Compound 144)

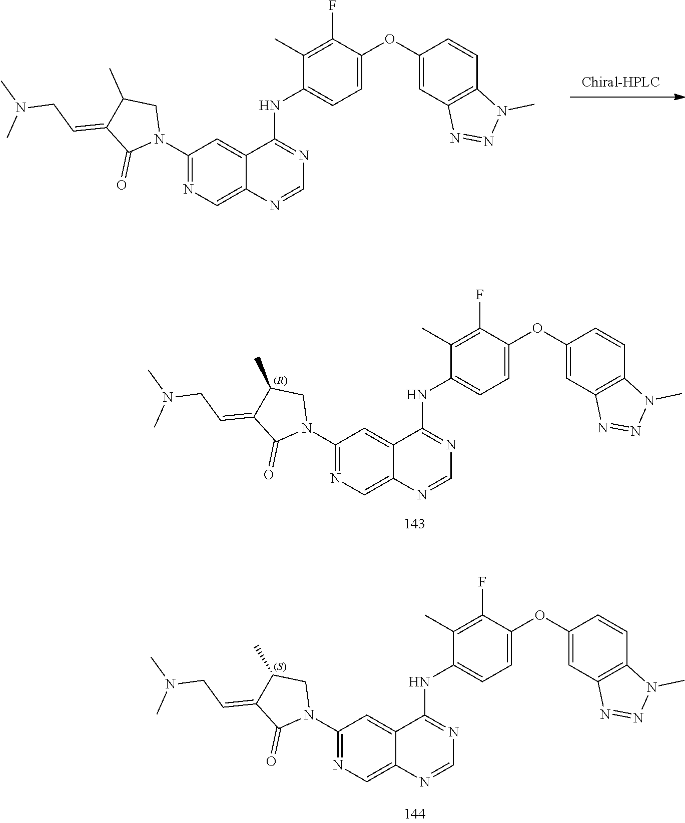

The racemic mixture of (E)-3-(2-(dimethylamino)ethylidene)-1-(4-((3-fluoro-2-methyl-4-((1-methyl-1H-benzo[d][1,2,3]triazol-5-yl)oxy)phenyl)amino)pyrido[3,4-d]pyrimidin-6-yl)-4-methylpyrrolidin-2-one (50.0 mg, 0.09 mmol) was separated by Prep-Chiral-HPLC with the following conditions (Column: CHIRAL ART Cellulose-SB, 2×25 cm, 5 μm; Mobile Phase A: Hex (0.5% 2M NH₃-MeOH)-HPLC, Mobile Phase B: IPA: DCM=1: 1-HPLC; Flow rate: 20 mL/min; Gradient: 30% B to 30% B in 13 min; Wave Length: 220/254 nm; RT1(min): 10.33; RT2(min): 12.40) to afford (R,E)-3-(2-(dimethylamino)ethylidene)-1-(4-((3-fluoro-2-methyl-4-((1-methyl-1H-benzo[d][1,2,3]triazol-5-yl)oxy)phenyl)amino)pyrido[3,4-d]pyrimidin-6-yl)-4-methylpyrrolidin-2-one (assumed, 3.6 mg, 14%) as a white solid and (S,E)-3-(2-(dimethylamino)ethylidene)-1-(4-((3-fluoro-2-methyl-4-((1-methyl-1H-benzo[d][1,2,3]triazol-5-yl)oxy)phenyl)amino)pyrido[3,4-d]pyrimidin-6-yl)-4-methylpyrrolidin-2-one (assumed, 2.1 mg, 8%) as a white solid.

The absolute stereochemistry of Enantiomers 1 and 2 was not assigned. The two enantiomeric structures that could be obtained from chiral separation of the enantiomeric mixture as described above are shown as Compounds 143 and 144 in Table 1.

(R,E)-3-(2-(dimethylamino)ethylidene)-1-(4-((3-fluoro-2-methyl-4-((1-methyl-1H-benzo[d][1,2,3]triazol-5-yl)oxy)phenyl)amino)pyrido[3,4-d]pyrimidin-6-yl)-4-methylpyrrolidin-2-one: RT1(min): 10.33, LCMS (ESI, m/z): [M+H]⁺=568.3. ¹H NMR (400 MHz, DMSO-d₆): δ 10.34 (s, 1H), 9.20 (s, 1H), 9.07 (s, 1H), 8.51 (s, 1H), 7.93 (d, J=8.4 Hz, 1H), 7.51-7.45 (m, 2H), 7.23-7.17 (m, 1H), 7.11-7.02 (m, 1H), 6.55-6.43 (m, 1H), 4.41 (s, 3H), 4.32-4.24 (m, 1H), 3.83-3.75 (m, 1H), 3.31-3.21 (m, 2H), 3.13-3.03 (m, 1H), 2.20-1.92 (m, 9H), 1.45-1.41 (m, 3H).

(S,E)-3-(2-(dimethylamino)ethylidene)-1-(4-((3-fluoro-2-methyl-4-((1-methyl-1H-benzo[d][1,2,3]triazol-5-yl)oxy)phenyl)amino)pyrido[3,4-d]pyrimidin-6-yl)-4-methylpyrrolidin-2-one: RT2(min): 12.40; LCMS (ESI, m/z): [M+H]⁺=568.3. ¹H NMR (400 MHz, DMSO-d₆): δ 10.35 (s, 1H), 9.20 (s, 1H), 9.08 (s, 1H), 8.52 (s, 1H), 7.94 (d, J=9.2 Hz, 1H), 7.51 (d, J=1.6 Hz, 1H), 7.46-7.44 (m, 1H), 7.21 (d, J=8.8 Hz, 1H), 7.08-7.04 (m, 1H), 6.53-6.50 (m, 1H), 4.35-4.33 (m, 3H), 4.27-4.23 (m, 1H), 3.83-3.76 (m, 1H), 3.33-3.21 (m, 2H), 3.15-3.09 (m, 1H), 2.21 (s, 6H), 2.13-2.08 (m, 3H), 1.36-1.23 (m, 3H).

### Example S125: Synthesis of (3E,4R)-1-[4-({2,5-fluoro-4-[(1-methyl-1,2,3-benzotriazol-5-yl)oxy]phenyl}amino)pyrido[3,4-d]pyrimidin-6-yl]-3-[2-(dimethylamino)ethylidene]-4-methylpyrrolidin-2-one and (3E,4S)-1-[4-({2,5-difluoro-4-[(1-methyl-1,2,3-benzotriazol-5-yl)oxy]phenyl}amino)pyrido[3,4-d]pyrimidin-6-yl]-3-[2-(dimethylamino)ethylidene]-4-methylpyrrolidin-2-one (Compound 145 and Compound 146)

### Step 1. Synthesis of 5-(2,5-difluoro-4-nitrophenoxy)-1-methyl-1,2,3-benzotriazole

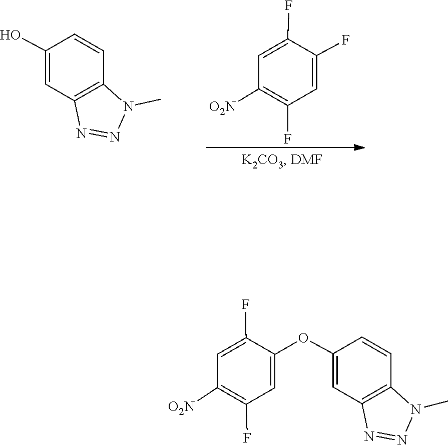 [SMILES: Cn1nnc2cc(O)ccc21 ; O=[N+]([O-])c1cc(F)c(F)cc1F ; Cn1nnc2cc(Oc3cc(F)c([N+](=O)[O-])cc3F)ccc21]

To a solution of 1-methyl-1,2,3-benzotriazol-5-ol (400.0 mg, 2.68 mmol) in DMF (13.0 mL) was added 1,2,4-trifluoro-5-nitrobenzene (569.9 mg, 3.22 mmol) and K₂CO₃ (1111.9 mg, 8.05 mmol) at 0° C. under N₂. The resulting mixture was stirred at 0° C. for 2 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with ethyl acetate/Petroleum ether (30/70, v/v) to afford 5-(2,5-difluoro-4-nitrophenoxy)-1-methyl-1,2,3-benzotriazole (698.3 mg, 85%) as a white solid. LCMS (ESI, m/z): [M+H]⁺=307.2.

### Step 2. Synthesis of 2,5-difluoro-4-[(1-methyl-1,2,3-benzotriazol-5-yl)oxy]aniline

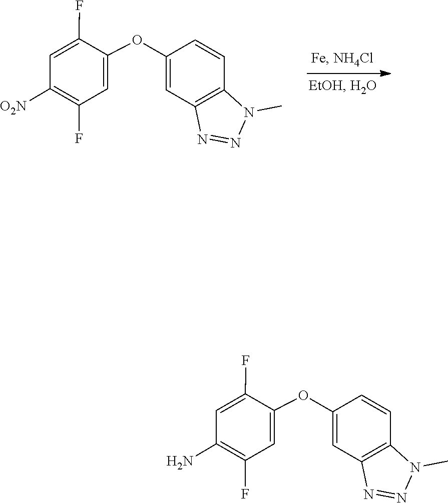 [SMILES: Cn1nnc2cc(Oc3cc(F)c([N+](=O)[O-])cc3F)ccc21 ; Cn1nnc2cc(Oc3cc(F)c(N)cc3F)ccc21]

To a solution of 5-(2,5-difluoro-4-nitrophenoxy)-1-methyl-1,2,3-benzotriazole (780.0 mg, 2.55 mmol) in EtOH (16.0 mL) and H₂O (4.0 mL) were added Fe (711.2 mg, 12.73 mmol) and NH₄Cl (681.5 mg, 12.73 mmol) at room temperature. The mixture was stirred at 80° C. for 16 h. After the reaction was completed, the mixture was filtered. The filtrate was concentrated under reduced pressure. The residue was purified by flash column chromatography with ethyl acetate/petroleum ether (90/10, v/v) to afford 2,5-difluoro-4-[(1-methyl-1,2,3-benzotriazol-5-yl)oxy]aniline (500.0 mg, 71%) as a brown solid. LCMS (ESI, m/z): [M+H]⁺=277.2.

### Step 3. Synthesis of 6-chloro-N-{2,5-difluoro-4-[(1-methyl-1,2,3-benzotriazol-5-yl)oxy]phenyl}pyrido[3,4-d]pyrimidin-4-amine

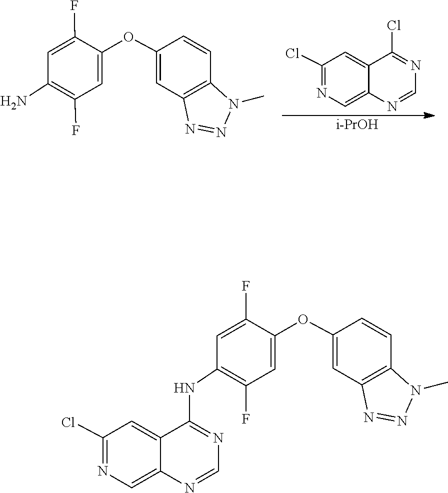 [SMILES: "i-PrOH" ; Cn1nnc2cc(Oc3cc(F)c(N)cc3F)ccc21 ; Clc1cc2c(Cl)ncnc2cn1 ; Cn1nnc2cc(Oc3cc(F)c(Nc4ncnc5cnc(Cl)cc45)cc3F)ccc21]

To a solution of 2,5-difluoro-4-[(1-methyl-1,2,3-benzotriazol-5-yl)oxy]aniline (200.0 mg, 0.72 mmol) in i-PrOH (5.0 mL) was added 4,6-dichloropyrido[3,4-d]pyrimidine (144.8 mg, 0.72 mmol) at room temperature. The mixture was stirred at room temperature for 16 h. After the reaction was completed, the resulting mixture was concentrated under reduced pressure. The residue was purified by flash column chromatography with dichloromethane/methanol (83/17, v/v) to afford 6-chloro-N-{2,5-difluoro-4-[(1-methyl-1,2,3-benzotriazol-5-yl)oxy]phenyl}pyrido[3,4-d]pyrimidin-4-amine (280.0 mg, 87%) as a yellow solid. LCMS (ESI, m/z): [M+H]⁺=440.1.

### Step 4. Synthesis of (E)-1-(4-((2,5-difluoro-4-((1-methyl-1H-benzo[d][1,2,3]triazol-5-yl)oxy)phenyl)amino)pyrido[3,4-d]pyrimidin-6-yl)-3-(2-(dimethylamino)ethylidene)-4-methylpyrrolidin-2-one

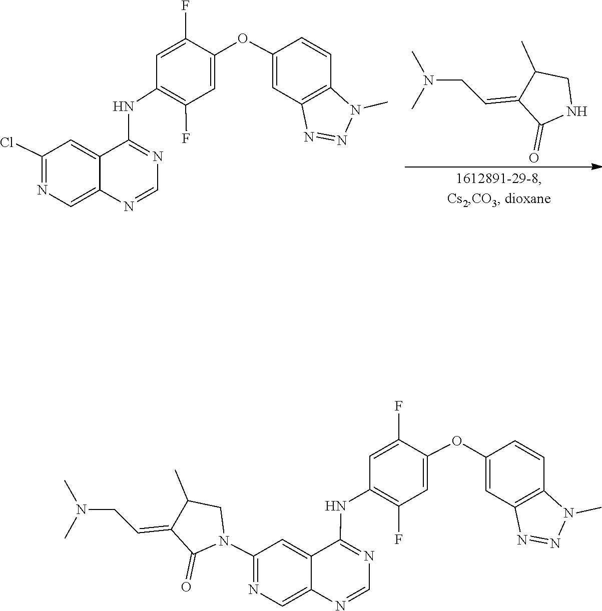

To a solution of 6-chloro-N-{2,5-difluoro-4-[(1-methyl-1,2,3-benzotriazol-5-yl)oxy]phenyl}pyrido[3,4-d]pyrimidin-4-amine (220.0 mg, 0.50 mmol) in dioxane (8.0 mL) was added (3E)-3-[2-(dimethylamino)ethylidene]-4-methylpyrrolidin-2-one (84.2 mg, 0.50 mmol), Pd-PEPPSI-IPentCl 2-methylpyridine (o-picoline) (84.2 mg, 0.10 mmol) and Cs₂CO₃ (488.9 mg, 1.50 mmol) at room temperature under N₂. The mixture was stirred at 100° C. for 2 h. After the reaction was completed, the mixture was filtered. The filtrate was concentrated under reduced pressure. The residue was purified by flash column chromatography with dichloromethane/methanol (91/9, v/v) to afford (E)-1-(4-((2,5-difluoro-4-((1-methyl-1H-benzo[d][1,2,3]triazol-5-yl)oxy)phenyl)amino)pyrido[3,4-d]pyrimidin-6-yl)-3-(2-(dimethylamino)ethylidene)-4-methylpyrrolidin-2-one (52.0 mg, 18%) as a white solid. LCMS (ESI, m/z): [M+H]⁺=572.2.

### Step 5. Synthesis of (3E,4R)-1-[4-({2,5-difluoro-4-[(1-methyl-1,2,3-benzotriazol-5-yl)oxy]phenyl}amino)pyrido[3,4-d]pyrimidin-6-yl]-3-[2-(dimethylamino)ethylidene]-4-methylpyrrolidin-2-one and (3E,4S)-1-[4-({2,5-difluoro-4-[(1-methyl-1,2,3-benzotriazol-5-yl)oxy]phenyl}amino)pyrido[3,4-d]pyrimidin-6-yl]-3-[2-(dimethylamino)ethylidene]-4-methylpyrrolidin-2-one (Compound 145 and Compound 146)

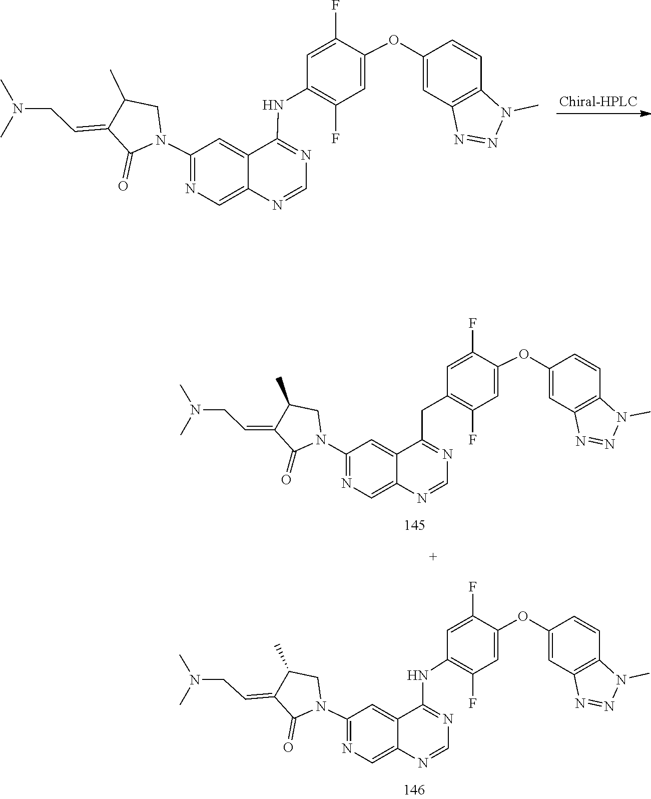

The racemic mixture of (E)-1-(4-((2,5-difluoro-4-((1-methyl-1H-benzo[d][1,2,3]triazol-5-yl)oxy)phenyl)amino)pyrido[3,4-d]pyrimidin-6-yl)-3-(2-(dimethylamino)ethylidene)-4-methylpyrrolidin-2-one (52.0 mg, 0.09 mmol) was separated by Prep-Chiral-HPLC with the following conditions: (Column: CHIRAL ART Cellulose-SB, 2×25 cm, 5 μm; Mobile Phase A: Hex (0.5% 2M NH₃-MeOH)-HPLC, Mobile Phase B: EtOH: DCM=1: 1-HPLC; Flow rate: 20 mL/min; Gradient: 30% B to 30% B in 12 min; Wave Length: 220/254 nm; RT1(min): 9.39; RT2(min): 11.33) to afford (3E)-1-[4-({2,5-difluoro-4-[(1-methyl-1,2,3-benzotriazol-5-yl)oxy]phenyl}amino)pyrido[3,4-d]pyrimidin-6-yl]-3-[2-(dimethylamino)ethylidene]-4-methylpyrrolidin-2-one Enantiomer 1 (RT1 9.39 min, 14.9 mg, 57%) as a white solid and (3E)-1-[4-({2,5-difluoro-4-[(1-methyl-1,2,3-benzotriazol-5-yl)oxy]phenyl}amino)pyrido[3,4-d]pyrimidin-6-yl]-3-[2-(dimethylamino)ethylidene]-4-methylpyrrolidin-2-one Enantiomer 2 (RT2 11.33 min, 14.8 mg, 57%) as a white solid. The absolute stereochemistry of Enantiomers 1 and 2 was not assigned. The two enantiomeric structures that could be obtained from chiral separation of the enantiomeric mixture as described above are shown as Compounds 145 and 146 in Table 1.

(3E)-1-[4-({2,5-difluoro-4-[(1-methyl-1,2,3-benzotriazol-5-yl)oxy]phenyl}amino)pyrido[3,4-d]pyrimidin-6-yl]-3-[2-(dimethylamino)ethylidene]-4-methylpyrrolidin-2-one Enantiomer 1: RT1(min): 9.39; LCMS (ESI, m/z): [M+H]⁺=572.3. ¹H NMR (400 MHz, DMSO-d₆): δ 10.44 (s, 1H), 9.19 (s, 1H), 9.12 (s, 1H), 8.60 (s, 1H), 7.96 (d, J=8.8 Hz, 1H), 7.74-7.70 (m, 1H), 7.65 (s, 1H), 7.50-7.47 (m, 1H), 7.28-7.23 (m, 1H), 6.54-6.51 (m, 1H), 4.34 (s, 3H), 4.27-4.23 (m, 1H), 3.83-3.81 (m, 1H), 3.25-3.21 (m, 2H), 3.15-3.09 (m, 1H), 2.22 (s, 6H), 1.24 (d, J=6.8 Hz, 3H).

(3E)-1-[4-({2,5-difluoro-4-[(1-methyl-1,2,3-benzotriazol-5-yl)oxy]phenyl}amino)pyrido[3,4-d]pyrimidin-6-yl]-3-[2-(dimethylamino)ethylidene]-4-methylpyrrolidin-2-one Enantiomer 2: RT2(min): 11.33; LCMS (ESI, m/z): [M+H]⁺=572.3. ¹H NMR (400 MHz, DMSO-d₆): δ 10.44 (s, 1H), 9.19 (s, 1H), 9.11 (s, 1H), 8.60 (s, 1H), 7.96 (d, J=9.2 Hz, 1H), 7.72-7.65 (m, 2H), 7.50-7.47 (m, 1H), 7.28-7.24 (m, 1H), 6.55-6.48 (m, 1H), 4.34 (s, 3H), 4.27-4.22 (m, 1H), 3.83-3.80 (m, 1H), 3.26-3.21 (m, 2H), 3.15-3.09 (m, 1H), 2.21 (s, 6H), 1.24 (d, J=6.8 Hz, 3H).

### Example S126: Synthesis of (3E,4R)-3 [2-(dimethylamino)ethylidene]-4-methyl-1-[4-({3-methyl-4-[(1-methyl-1,2,3-benzotriazol-5 yl)oxy]phenyl}amino)pyrido[3,2-d]pyrimidin-6-yl]pyrrolidin-2-one and (3E,4S)-3-[2-(dimethylamino)ethylidene]-4-methyl-1-[4-({3-methyl-4-[(1-methyl-1,2,3-benzotriazol-5-yl)oxy]phenyl}amino)pyrido[3,2-d]pyrimidin-6-yl]pyrrolidin-2-one (Compound 151 and Compound 152)

### Step 1. Synthesis of (E)-3-(2-(dimethylamino)ethylidene)-4-methyl-1-(4-((3-methyl-4-((1-methyl-1H-benzo[d][1,2,3]triazol-5-yl)oxy)phenyl)amino)pyrido[3,2-d]pyrimidin-6-yl)pyrrolidin-2-one

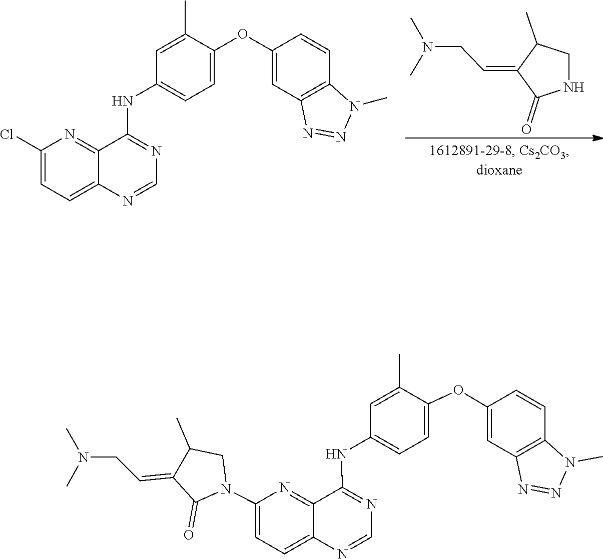

To a solution of 6-chloro-N-(3-methyl-4-((1-methyl-1H-benzo[d][1,2,3]triazol-5-yl)oxy)phenyl)pyrido[3,2-d]pyrimidin-4-amine (100.0 mg, 0.23 mmol) in 1,4-dioxane (10.0 mL) was added (E)-3-(2-(dimethylamino)ethylidene)-4-methylpyrrolidin-2-one (39.8 mg, 0.25 mmol), Cs₂CO₃ (53.6 mg, 0.70 mmol) and Pd-PEPPSI-IPentCl 2-methylpyridine (o-picoline) (39.5 mg, 0.04 mmol) at room temperature under N₂. The resulting mixture was stirred at 100° C. for 16 h. After the reaction was completed, the mixture was filtered. The filtrate was concentrated under reduced pressure. The residue was purified by flash column chromatography with CH₂Cl₂/MeOH (90/10, v/v) to afford (E)-3-(2-(dimethylamino)ethylidene)-4-methyl-1-(4-((3-methyl-4-((1-methyl-1H-benzo[d][1,2,3]triazol-5-yl)oxy)phenyl)amino)pyrido[3,2-d]pyrimidin-6-yl)pyrrolidin-2-one (55.0 mg, 32%) as a light yellow solid. LCMS (ESI, m/z): [M+H]⁺=550.1.

### Step 2. Synthesis of (3E,4R)-3-[2-(dimethylamino)ethylidene]-4-methyl-1-[4-({3-methyl-4-[(1-methyl-1,2,3-benzotriazol-5-yl)oxy]phenyl}amino)pyrido[3,2-d]pyrimidin-6-yl]pyrrolidin-2-one and (3E,4S)-3-[2-(dimethylamino)ethylidene]-4-methyl-1-[4-({3-methyl-4-[(1-methyl-1,2,3-benzotriazol-5-yl)oxy]phenyl}amino)pyrido[3,2-d]pyrimidin-6-yl]pyrrolidin-2-one (Compound 151 and Compound 152)

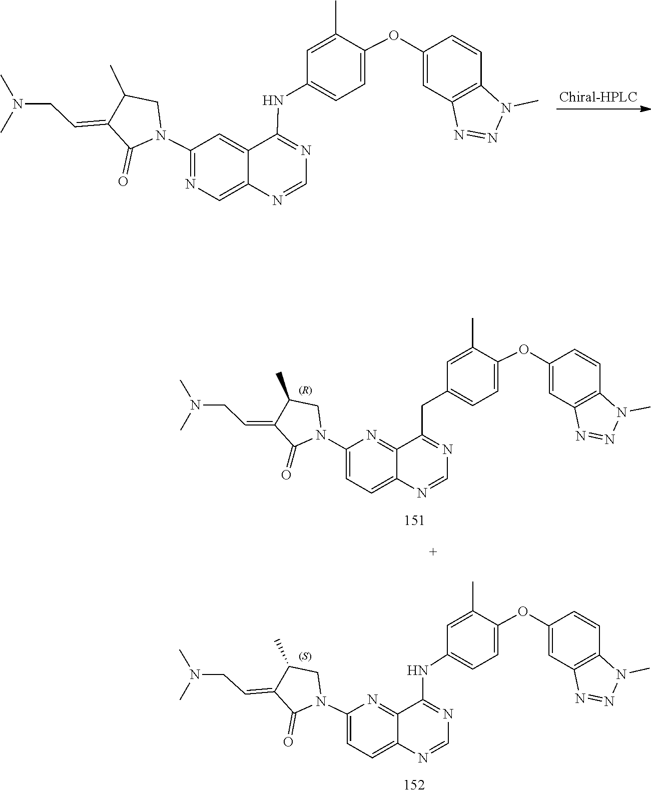

The racemic mixture of (3E)-3-[2-(dimethylamino)ethylidene]-4-methyl-1-[4-({3-methyl-4-[(1-methyl-1,2,3-benzotriazol-5-yl)oxy]phenyl}amino)pyrido[3,2-d]pyrimidin-6-yl]pyrrolidin-2-one (55.0 mg, 0.055 mmol) was separated by Prep-Chiral-HPLC with the following conditions (Column: CHIRAL ART Amylose-SA, 2×25 cm, 5 μm; Mobile Phase A: Hex (0.5% 2M NH₃-MeOH)-HPLC, Mobile Phase B: MeOH: DCM=1: 1-HPLC; Flow rate: 20 mL/min; Gradient: 50% B to 50% B in 15 min; Wave Length: 220/254 nm; RT1(min): 6.57; RT2(min): 12.42) to afford 3-[2-(dimethylamino)ethylidene]-4-methyl-1-[4-({3-methyl-4-[(1-methyl-1,2,3-benzotriazol-5-yl)oxy]phenyl}amino)pyrido[3,2-d]pyrimidin-6-yl]pyrrolidin-2-one Enantiomer 1 (RT1 6.57 min, 9.2 mg, 33%) as a yellow solid and 3-[2-(dimethylamino)ethylidene]-4-methyl-1-[4-({3-methyl-4-[(1-methyl-1,2,3-benzotriazol-5-yl)oxy]phenyl}amino)pyrido[3,2-d]pyrimidin-6-yl]pyrrolidin-2-one Enantiomer 2 (RT2 12.42 min, 10.0 mg, 36%) as a yellow solid. The absolute stereochemistry of Enantiomers 1 and 2 was not assigned. The two enantiomeric structures that could be obtained from chiral separation of the enantiomeric mixture as described above are shown as Compounds 151 and 152 in Table 1.

3-[2-(dimethylamino)ethylidene]-4-methyl-1-[4-({3-methyl-4-[(1-methyl-1,2,3-benzotriazol-5-yl)oxy]phenyl}amino)pyrido[3,2-d]pyrimidin-6-yl]pyrrolidin-2-one Enantiomer 1: RT1(min): 6.57; LCMS (ESI, m/z): [M+H]⁺=550.4. ¹H NMR (400 MHz, DMSO-d₆): δ 9.58 (s, 1H), 8.99 (d, J=9.2 Hz, 1H), 8.61 (s, 1H), 8.26 (d, J=9.2 Hz, 1H), 7.91-7.84 (m, 3H), 7.38-7.35 (m, 1H), 7.28 (s, 1H), 7.05 (d, J=8.4 Hz, 1H), 6.57-6.54 (m, 1H), 4.34-4.31 (m, 4H), 4.15-4.12 (m, 1H), 3.26-3.11 (m, 3H), 2.26-2.22 (m, 9H), 1.27 (d, J=7.2 Hz, 3H).

3-[2-(dimethylamino)ethylidene]-4-methyl-1-[4-({3-methyl-4-[(1-methyl-1,2,3-benzotriazol-5-yl)oxy]phenyl}amino)pyrido[3,2-d]pyrimidin-6-yl]pyrrolidin-2-one Enantiomer 2: RT2(min): 12.42; LCMS (ESI, m/z): [M+H]⁺=550.4. ¹H NMR (400 MHz, DMSO-d₆): δ 9.57 (s, 1H), 8.99 (d, J=9.2 Hz, 1H), 8.61 (s, 1H), 8.26 (d, J=9.6 Hz, 1H), 7.91-7.84 (m, 3H), 7.38-7.35 (m, 1H), 7.28 (s, 1H), 7.05 (d, J=8.4 Hz, 1H), 6.57-6.54 (m, 1H), 4.34-4.31 (m, 4H), 4.15-4.12 (m, 1H), 3.26-3.11 (m, 3H), 2.26-2.22 (m, 9H), 1.27 (d, J=7.2 Hz, 3H).

### Example S127: Synthesis of (R,E)-1-(4-((4-([1,2,4]triazolo[1,5-a]pyridin-7-yloxy)-3-fluorophenyl)amino)pyrido[3,2-d]pyrimidin-6-yl)-3-(2-(dimethylamino)ethylidene)-4-methylpyrrolidin-2-one and (S,E)-1-(4-((4-([1,2,4]triazolo[1,5-a]pyridin-7-yloxy)-3-fluorophenyl)amino)pyrido[3,2-d]pyrimidin-6-yl)-3-(2-(dimethylamino)ethylidene)-4-methylpyrrolidin-2-one (Compound 153 and Compound 154)

### Step 1. Synthesis of (E)-1-(4-((4-([1,2,4]triazolo[1,5-a]pyridin-7-yloxy)-3-fluorophenyl)amino)pyrido[3,2-d]pyrimidin-6-yl)-3-(2-(dimethylamino)ethylidene)-4-methylpyrrolidin-2-one

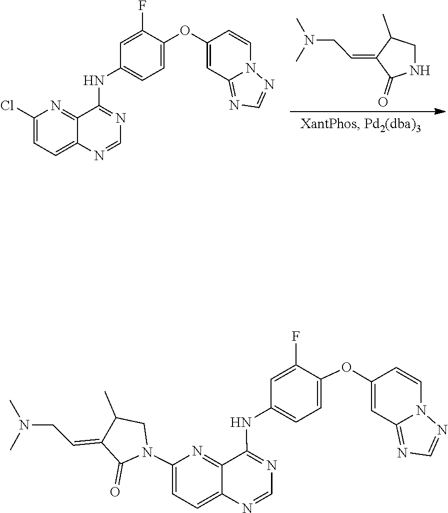

To a solution of N-(4-([1,2,4]triazolo[1,5-a]pyridin-7-yloxy)-3-fluorophenyl)-6-chloropyrido[3,2-d]pyrimidin-4-amine (170.0 mg, 0.41 mmol) in dioxane (10.0 mL) was added (E)-3-(2-(dimethylamino)ethylidene)-4-methylpyrrolidin-2-one (70.1 mg, 0.41 mmol), Cs₂CO₃ (543.3 mg, 1.66 mmol), XantPhos (48.2 mg, 0.08 mmol) and Pd₂(dba)₃ (38.1 mg, 0.04 mmol) at room temperature under N₂. The mixture was stirred at 120° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with dichloromethane/methanol (80/20, v/v) to afford (E)-1-(4-((4-([1,2,4]triazolo[1,5-a]pyridin-7-yloxy)-3-fluorophenyl)amino)pyrido[3,2-d]pyrimidin-6-yl)-3-(2-(dimethylamino)ethylidene)-4-methylpyrrolidin-2-one (55.0 mg, 16%) as a white solid. LCMS (ESI, m/z): [M+H]⁺=540.2.

### Step 2. Synthesis of (R,E)-1-(4-((4-([1,2,4]triazolo[1,5-a]pyridin-7-yloxy)-3-fluorophenyl)amino)pyrido[3,2-d]pyrimidin-6-yl)-3-(2-(dimethylamino)ethylidene)-4-methylpyrrolidin-2-one and (S,E)-1-(4-((4-([1,2,4]triazolo[1,5-a]pyridin-7-yloxy)-3-fluorophenyl)amino)pyrido[3,2-d]pyrimidin-6-yl)-3-(2-(dimethylamino)ethylidene)-4-methylpyrrolidin-2-one (Compound 153 and Compound 154)

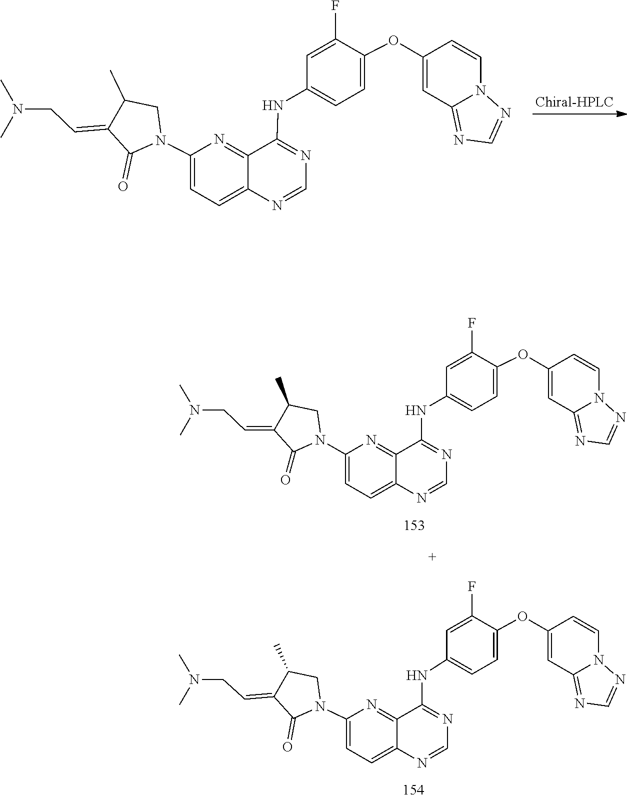 [SMILES: CC1CN(c2ccc3ncnc(Nc4ccc(Oc5ccn6ncnc6c5)c(F)c4)c3n2)C(=O)/C1=C/CN(C)C ; C[C@H]1CN(c2ccc3ncnc(Nc4ccc(Oc5ccn6ncnc6c5)c(F)c4)c3n2)C(=O)/C1=C/CN(C)C ; C[C@@H]1CN(c2ccc3ncnc(Nc4ccc(Oc5ccn6ncnc6c5)c(F)c4)c3n2)C(=O)/C1=C/CN(C)C]

The racemic mixture of (E)-1-(4-((4-([1,2,4]triazolo[1,5-a]pyridin-7-yloxy)-3-fluorophenyl)amino)pyrido[3,2-d]pyrimidin-6-yl)-3-(2-(dimethylamino)ethylidene)-4-methylpyrrolidin-2-one (55.0 mg, 0.10 mmol) was separated by chiral-HPLC with the following conditions: (Column: CHIRAL ART Cellulose-SB, 2×25 cm, 5 μm; Mobile Phase A: Hex (0.5% 2M NH₃-MeOH)-HPLC, Mobile Phase B: MeOH: DCM=1: 1-HPLC; Flow rate: 20 mL/min; Gradient: 25% B to 25% B in 16 min; Wave Length: 220/254 nm; RT1(min): 10.63; RT2(min): 13.89) to afford (E)-1-(4-((4-([1,2,4]triazolo[1,5-a]pyridin-7-yloxy)-3-fluorophenyl)amino)pyrido[3,2-d]pyrimidin-6-yl)-3-(2-(dimethylamino)ethylidene)-4-methylpyrrolidin-2-one (RT1 10.63 min, 12.1 mg, 44%) as a white solid and (E)-1-(4-((4-([1,2,4]triazolo[1,5-a]pyridin-7-yloxy)-3-fluorophenyl)amino)pyrido[3,2-d]pyrimidin-6-yl)-3-(2-(dimethylamino)ethylidene)-4-methylpyrrolidin-2-one (RT2 13.89 min, 17.1 mg, 62%) as a white solid. The absolute stereochemistry of Enantiomers 1 and 2 was not assigned. The two enantiomeric structures that could be obtained from chiral separation of the enantiomeric mixture as described above are shown as Compounds 153 and 154 in Table 1.

(E)-1-(4-((4-([1,2,4]triazolo[1,5-a]pyridin-7-yloxy)-3-fluorophenyl)amino)pyrido[3,2-d]pyrimidin-6-yl)-3-(2-(dimethylamino)ethylidene)-4-methylpyrrolidin-2-one Enantiomer 1: RT1(min): 10.63; LCMS (ESI, m/z): [M+H]⁺=540.4. ¹H NMR (400 MHz, DMSO-d₆): 9.71 (s, 1H), 9.02-8.97 (m, 2H), 8.70 (s, 1H), 8.42 (s, 1H), 8.32-8.29 (m, 2H), 7.94 (d, J=8.8 Hz, 1H), 7.54-7.50 (m, 1H), 7.12-7.10 (m, 1H), 7.03 (d, J=2.8 Hz, 1H), 6.59-6.56 (m, 1H), 4.36-4.31 (m, 1H), 4.15-4.12 (m, 1H), 3.29-3.12 (m, 3H), 2.22 (s, 6H), 1.28 (d, J=7.2 Hz, 3H).

(E)-1-(4-((4-([1,2,4]triazolo[1,5-a]pyridin-7-yloxy)-3-fluorophenyl)amino)pyrido[3,2-d]pyrimidin-6-yl)-3-(2-(dimethylamino)ethylidene)-4-methylpyrrolidin-2-one Enantiomer 2: RT2(min): 13.89; LCMS (ESI, m/z): [M+H]⁺=540.2. ¹H NMR (400 MHz, DMSO-d₆): δ 9.72 (s, 1H), 9.03-8.97 (m, 2H), 8.70 (s, 1H), 8.42 (s, 1H), 8.33-8.29 (m, 2H), 7.94 (d, J=8.8 Hz, 1H), 7.54-7.50 (m, 1H), 7.12-7.10 (m, 1H), 7.06-7.04 (m, 1H), 6.58-6.55 (m, 1H), 4.36-4.31 (m, 1H), 4.15-4.12 (m, 1H), 3.27-3.11 (m, 3H), 2.22 (s, 6H), 1.29-1.24 (m, 3H).

### Example S128: Synthesis of (3E)-3 [2-(dimethylamino)ethylidene]-1-[4-({4-[(5 fluoropyridin-3 yl)oxy]-3-methylphenyl}amino)pyrido[3,4-d]pyrimidin-6-yl]pyrrolidin-2-one (Compound 155)

### Step 1. Synthesis of 6-chloro-N-{4-[(5-fluoropyridin-3-yl)oxy]-3-methylphenyl}pyrido[3,4-d]pyrimidin-4-amine

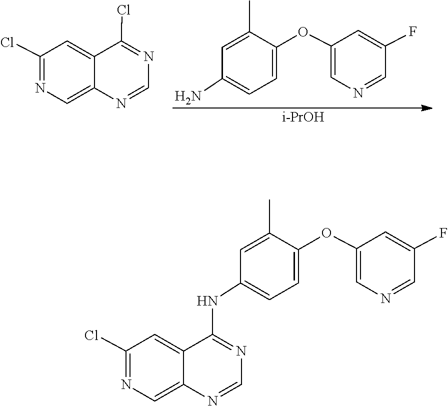 [SMILES: "i-PrOH" ; Clc1cc2c(Cl)ncnc2cn1 ; Cc1cc(N)ccc1Oc1cncc(F)c1 ; Cc1cc(Nc2ncnc3cnc(Cl)cc23)ccc1Oc1cncc(F)c1]

A mixture of 4,6-dichloropyrido[3,4-d]pyrimidine (500.0 mg, 2.50 mmol) and 4-[(5-fluoropyridin-3-yl)oxy]-3-methylaniline (545.5 mg, 2.50 mmol) in i-PrOH (20.0 mL) was stirred at room temperature for 2 h. After the reaction was completed, the resulting mixture was concentrated under vacuum. The residue was purified by flash column chromatography with CH₂Cl₂/CH₃OH (84/16, v/v) to afford 6-chloro-N-{4-[(5-fluoropyridin-3-yl)oxy]-3-methylphenyl}pyrido[3,4-d]pyrimidin-4-amine (800.0 mg, 83%) as a yellow solid. LCMS (ESI, m/z): [M+H]⁺=382.1.

### Step 2. Synthesis of (3E)-3-[2-(dimethylamino)ethylidene]-1-[4-({4-[(5-fluoropyridin-3-yl)oxy]-3-methylphenyl}amino)pyrido[3,4-d]pyrimidin-6-yl]pyrrolidin-2-one (Compound 155)

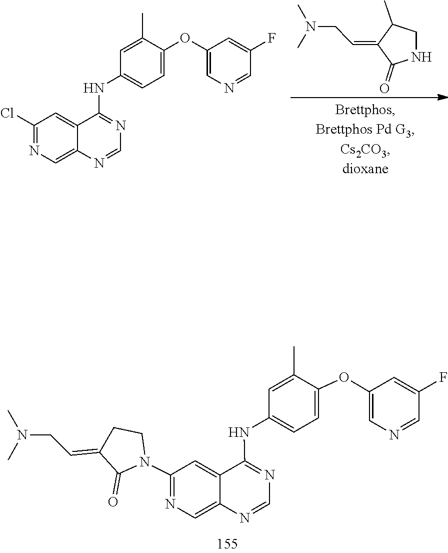

A mixture of 6-chloro-N-{4-[(5-fluoropyridin-3-yl)oxy]-3-methylphenyl}pyrido[3,4-d]pyrimidin-4-amine (150.0 mg, 0.39 mmol), (3E)-3-[2-(dimethylamino)ethylidene]pyrrolidin-2-one (60.5 mg, 0.39 mmol), BrettPhos (42.1 mg, 0.07 mmol), BrettPhos Pd G3 (35.6 mg, 0.03 mmol) and Cs₂CO₃ (384.0 mg, 1.17 mmol) in dioxane (10.0 mL) was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (74/26, v/v) and then purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 35% B to 45% B in 8 min, 254 nm) to afford (3E)-3-[2-(dimethylamino)ethylidene]-1-[4-({4-[(5-fluoropyridin-3-yl)oxy]-3-methylphenyl}amino)pyrido[3,4-d]pyrimidin-6-yl]pyrrolidin-2-one (Compound 155) (51.1 mg, 25%) as a white solid. LCMS (ESI, m/z): [M+H]⁺=500.1. ¹H NMR (400 MHz, DMSO-d₆): δ 10.30 (s, 1H), 9.20 (s, 1H), 9.08 (s, 1H), 8.63 (s, 1H), 8.35 (d, J=2.4 Hz, 1H), 8.23 (s, 1H), 7.83-7.76 (m, 2H), 7.34-7.31 (m, 1H), 7.13 (d, J=8.8 Hz, 1H), 6.55-6.52 (m, 1H), 4.19-4.15 (m, 2H), 3.10 (d, J=6.8 Hz, 2H), 2.88-2.83 (m, 2H), 2.23-2.18 (m, 9H).

### Example S129: Synthesis of (E)-1-(4-((4-([1,2,4]triazolo[1,5-a]pyridin-7-yloxy)-2-fluorophenyl)amino)pyrido[3,2-d]pyrimidin-6-yl)-3-(2-(dimethylamino)ethylidene)pyrrolidin-2-one (Compound 156)

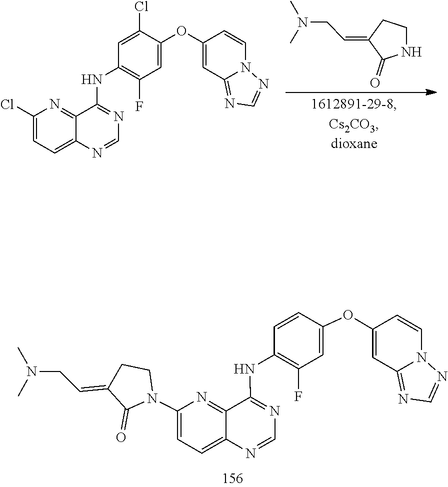 [SMILES: Fc1cc(Oc2ccn3ncnc3c2)c(Cl)cc1Nc1ncnc2ccc(Cl)nc12 ; CN(C)C/C=C1\CCNC1=O ; CN(C)C/C=C1\CCN(c2ccc3ncnc(Nc4ccc(Oc5ccn6ncnc6c5)cc4F)c3n2)C1=O]

To a solution of N-(4-([1,2,4]triazolo[1,5-a]pyridin-7-yloxy)-5-chloro-2-fluorophenyl)-6-chloropyrido[3,2-d]pyrimidin-4-amine (0.2 g, 0.45 mmol) in 1,4-dioxane (10.0 mL) was added Cs₂CO₃ (0.4 g, 1.35 mmol), Pd-PEPPSI-IPentCl 2-methylpyridine (o-picoline) (38.0 mg, 0.04 mmol) and (E)-3-(2-(dimethylamino)ethylidene)pyrrolidin-2-one (0.1 g, 0.68 mmol) at room temperature under N₂. The resulting mixture was stirred at 80° C. for 16 h. After the reaction was completed, the mixture was cooled down to room temperature and filtered. The filtrate was concentrated under reduced pressure. The residue was purified by flash chromatography with CH₂Cl₂/MeOH (85/15, v/v) and then purified by Prep-HPLC with the following conditions: (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 40% B to 50% B in 8 min; Wave Length: 254 nm) to afford (E)-1-(4-((4-([1,2,4]triazolo[1,5-a]pyridin-7-yloxy)-2-fluorophenyl)amino)pyrido[3,2-d]pyrimidin-6-yl)-3-(2-(dimethylamino)ethylidene)pyrrolidin-2-one (Compound 156) (5.8 mg, 2%) as a yellow solid. LCMS (ESI, m/z): [M+H]⁺=526.2. ¹H NMR (400 MHz, DMSO-d₆): δ 9.61 (s, 1H), 9.02-8.99 (m, 2H), 8.59 (s, 1H), 8.45 (s, 1H), 8.31-8.28 (m, 1H), 8.09-8.04 (m, 1H), 7.43-7.40 (m, 1H), 7.22-7.20 (m, 2H), 7.10-7.07 (m, 1H), 6.59-6.58 (m, 1H), 4.30-4.28 (m, 2H), 3.11 (d, J=6.8 Hz, 2H), 2.92-2.85 (m, 2H), 2.21 (s, 6H).

### BIOLOGICAL EXAMPLES

### Example B1. Cell Viability Assays

Cells were treated with compounds, and cell viability was measured as a metric of kinase inhibition.

BT-474, A431, MDA-MB-175VII, NCI-H1781, MCF7, and Ba/F3 cell lines were tested. The Ba/F3 cell line is IL-3-dependent mouse cell line derived from the C3H mouse strain. Ba/F3 cell lines were engineered to express human ERBB2 or EGFR kinases, rendering the cells IL-3 independent. The lines were generated via retroviral transduction utilizing a Moloney murine leukemia virus (MMLV) promoter, and constructs are stably integrated into the cell genome. The sequences of the ERBB2 and EGFR genes used were NCBI Reference Sequences NM_004448.3 and NM_005228.3, respectively.

BT-474, A431, MDA-MB-175VII, NCI-H1781 and MCF7 cells were grown in the appropriate growth medium as described in Table B2 below, and harvested at 50-80% confluence. BT-474, A431, MDA-MB-175VII, NCI-H1781 and MCF7 cells were counted and seeded at 2,000 or 1,500 cells per well in 384-well tissue culture plates (see Table B2). Similarly, Ba/F3 cell lines engineered to express EGFR, ERBB2, or ERBB2 mutants were grown, harvested, counted and seeded at 3000 cells per well in 96-well plates. A subset of wells contained media only (low control, “LC”).

Table B1 provides the growth media and number of cells seeded per well for the each cell line.

TABLE B1

| | | Number of cells |
|---|---|---|
| Cell Line | Growth Medium | seeded per well |
| BT-474 | Dulbecco's Modified Eagle | 2000 |
| | Medium (DMEM) + 10% | |
| | fetal bovine serum (FBS) | |
| A431 | DMEM + 10% FBS | 1500 |
| MDA-MB-175VII | RPMI + 10% FBS | 2000 |
| NCI-H1781 | RPMI + 10% FBS | 1500 |
| MCF7 | EMEM + 10% FBS + | 2000 |
| | 100 ng/ml human insulin | |
| Ba/F3 | RPMI + 10% FBS | 3000 |

Plates were placed in a 37° C., 5% CO₂ incubator for 72 hours. Plates were then removed from the incubator and equilibrated for 15 minutes at room temperature. 40 μl of CellTiter Glo reagent (Promega) was added to measure the relative level of metabolically active cells by quantifying intracellular ATP concentrations. Plates were incubated for 30 minutes at room temperature, and luminescence was measured. Percent viability was normalized to a vehicle control only using the following formula: % viability=100×(Lumₛₐₘₚₗₑ−Lum_{LC})/(Lum_{HC}−Lum^{LC}). IC₅₀ values were calculated using XLFit software or Prism (GraphPad Software), as shown in Tables B2-B5, below. Graphical curves were fitted using a nonlinear regression model with a sigmoidal dose response.

TABLE B2

| | Compound (IC₅₀, nM) | | | | |
|---|---|---|---|---|---|
| Cell Line | Tucatinib | Neratinib | 1 | 3 | 4 |
| BT-474 | 41.8 | 2.2 | 7.45 | 19.7 | 16.3 |
| MDA MB 175VII | 89 | 7.1 | 15.0 | * | * |
| A431 | >10,000 | 43.3 | 5796 | 1137 | 1278 |
| Ba/F3 ERBB2 wt | 26.5 | 1.1 | 2.3 | 11 | 3.9 |
| Ba/F3 ERBB2 A775_G776insYVMA | 356 | 3.5 | 28 | 202 | 16 |
| Ba/F3 EGFR wt | >10,000 | 287 | >10,000 | * | * |
| Ba/F3 ERBB2-G776VC | 193 | 3.9 | 16 | 40 | 7.5 |
| Ba/F3 ERBB2-P780_Y781insGSP | 25.3 | 3.4 | 3.4 | 13 | 2.6 |
| Ba/F3 ERBB2-V777_G778insGC | 12.5 | 0.79 | 0.82 | 3.8 | 1.8 |
| Ba/F3 ERBB2-V777L | 11 | 0.73 | 0.41 | 4.7 | 1.3 |
| Ba/F3 ERBB2-T798M | 1367 | 20 | 151 | 656 | 154 |
| Ba/F3 ERBB2-L755S | 228 | 6.4 | 13 | 172 | 14 |
| Ba/F3 ERBB2-L869R | 63.5 | 2.3 | 3.6 | 47 | 4.8 |
| BaF3-EGFR-V769_D770insASV | * | 62 | 4510 | * | * |
| BaF3_EGFR-D770-N771insSVD | * | 108 | 4056 | * | * |
| BaF3_EGFR-H773_V774insNPH | * | 481 | 9830 | * | * |

* not evaluated

TABLE B3

| | Compound (IC₅₀, nM) | | | | | |
|---|---|---|---|---|---|---|
| Cell Line | 2 | 5 | 6 | 7 | 8 | 9 |
| BT-474 | 19.5 | 27.8 | 16.1 | 31.7 | 52.9 | 44.7 |
| A431 | * | >10.0E+03 | >6.52E+03 | >10.0E+03 | 7500 | >10.0E+03 |
| Ba/F3 ERBB2 | * | 236 | >1.00E+03 | >1.00E+03 | * | * |
| A775_G776insYVMA | | | | | | |

* not evaluated

TABLE B4

| | Compound (IC₅₀, nM) | | | | | | |
|---|---|---|---|---|---|---|---|
| Cell Line | 10 | 11 | 12 | 14 | 15 | 16 | 18 |
| BT-474 | 27.1 | 7670 | 20.4 | 4.32 | 3.27 | 4.67 | 3.73 |
| A431 | 673 | 6510 | 7650 | * | * | * | * |
| Ba/F3 ERBB2 | * | * | 113 | * | 245 | 168.6 | 77.15 |
| A775_G776insYVMA | | | | | | | |

* not evaluated

TABLE B5

| | Ba/F3 ERBB2 |
|---|---|
| Compound | YVMA, IC₅₀, nM |
| 25 | >500 |
| 26 | 8.61 |
| 27 | 11.3 |
| 28 | 5.32 |
| 29 | 31.5 |
| 30 | 17.4 |
| 31 | 3.78 |
| 32 | 134 |
| 33 | 15.8 |
| Example S33, | 7.57 |
| Enantiomer # 1 | |
| Example S33, | 7.6 |
| Enantiomer # 2 | |
| 36 | 80.5 |
| 37 | 17.4 |
| 38 | 2 |
| 39 | 14.6 |
| 40 | >500 |
| 41 | 29 |
| Example S40, | 3.47 |
| Enantiomer # 1 | |
| Example S40, | 68.1 |
| Enantiomer # 2 | |
| 44 | 14.5 |
| 45 | 1.79 |
| 46 | 53.9 |
| 47 | 4.08 |
| 48 | 0.837 |
| 49 | 0.999 |
| 50 | 46.4 |
| 51 | 24.1 |
| 52 | 21.9 |
| 53 | 2.05 |
| 54 | 5.37 |
| 55 | 23 |
| 56 | >500 |
| Example S111, | 18.4 |
| Enantiomer #1 | |
| Example S111, | 10.8 |
| Enantiomer #2 | |
| 59 | 13.4 |
| 60 | 22.5 |
| 61 | 7.15 |
| 62 | 4.36 |
| Example S58, | 1.46 |
| Enantiomer # 1 | |
| Example S58, | 13.3 |
| Enantiomer # 2 | |
| 65 | 36.4 |
| 66 | 1.95 |
| 67 | 1.82 |
| 68 | 3.81 |
| 69 | 3.57 |
| 70 | 4.29 |
| 71 | 2.56 |
| 72 | 5.37 |
| 73 | 22 |
| 74 | 5.17 |
| 75 | 10.4 |
| 76 | >500 |
| 77 | 3.61 |
| 78 | 9.92 |
| 79 | 2.92 |
| 80 | 35.5 |
| 81 | 3.86 |
| 82 | 6.39 |
| 83 | 4.73 |
| 84 | 45 |
| Example S79, | 32.1 |
| Enantiomer #1 | |
| Example S79, | 13.7 |
| Enantiomer #2 | |
| 87 | 23 |
| 88 | 45.9 |
| 89 | 1.87 |
| 90 | 10.8 |
| 91 | 2.08 |
| 92 | 3.12 |
| Example S86, | 2.59 |
| Enantiomer #1 | |
| Example S86, | 240 |
| Enantiomer #2 | |
| 95 | 5.2 |
| 96 | 18.6 |
| 98 | 1.81 |
| 99 | 6.64 |
| 100 | 3.19 |
| 101 | 4.32 |
| 102 | 7.51 |
| Example S108, | 54 |
| Enantiomer #1 | |
| Example S108, | 4.88 |
| Enantiomer #2 | |
| 105 | 2.17 |
| 106 | 2.99 |
| 107 | 81.3 |
| 108 | 11.1 |
| 113 | >500 |
| 114 | 24.3 |
| 117 | 2 |
| 118 | 1.55 |
| 119 | 1.18 |
| Example S97, | 45.6 |
| Enantiomer #1 | |
| Example S97, | 4.12 |
| Enantiomer #2 | |
| Example S98, | 1.13 |
| Enantiomer #1 | |
| Example S98, | 71.2 |
| Enantiomer #2 | |
| 124 | 34.2 |
| 125 | 56 |
| 126 | 34 |
| 127 | 24.3 |
| 128 | 7.9 |
| 129 | 19.3 |
| 130 | 17.8 |
| Example S115, | 11.3 |
| Enantiomer #1 | |
| Example S115, | 286 |
| Enantiomer #2 | |
| 133 | 16.9 |
| Example S117, | 12 |
| Enantiomer #1 | |
| Example S117, | >500 |
| Enantiomer #2 | |
| 136 | 18 |
| 137 | 473 |
| 138 | 99.6 |
| 139 | 15.7 |
| 140 | 78.1 |
| 141 | 28.5 |
| 142 | 5.8 |
| Example S125, | >500 |
| Enantiomer #1 | |
| Example S125, | 27 |
| Enantiomer #2 | |
| Example S126, | 2.04 |
| Enantiomer #1 | |
| Example S126, | 205 |
| Enantiomer #2 | |
| Example S127, | 2.88 |
| Enantiomer #1 | |
| Example S127, | 418 |
| Enantiomer #2 | |
| 155 | 35.4 |
| 156 | 23.3 |

### Example B2. Detection of Phosphorylated ERBB2 (pERBB2) and Phosphorylated EGFR (pEGFR)

BT-474 cells were seeded into a 96-well at 2.0*10⁴ cells/100 μl/well.

Compounds were dissolved and serially diluted in DMSO. The compounds were then were added, mixed, and incubated for four hours at 37° C., 5% CO₂. Compounds were added using four-fold dilutions at final concentrations ranging from 10 μM to 0.01 nM.

Following the four hour incubation with compounds, cell lysates were prepared.

Plates were centrifuged for 5 min at 3000 RPM, and supernatant was removed from each well. Cells were washed 3 times by resuspension in 150 μl PBS, followed by centrifugation and removal of the supernatant, as above. 100 μl of cell lysis buffer (Boston BioProducts, cat #BP-115D) supplied with 1× complete ULTRA cocktail inhibitor (Thermo Scientific™, cat #78443) was then added to the washed cells. Cells were incubated with lysis buffer for 1 hour at 4° C., and then stored at −80° C.

Enzyme-linked immunosorbent assays (ELISA) were performed to measure phosphorylated ERBB2 levels. A capture antibody able to detect phosphorylated and non-phosphorylated ERBB2 (R&D Systems, cat #841425) was added to ELISA plates and incubated at 4° C. overnight. The next day, plates were washed with PBS+0.05% Tween20 (PBST). 150 μl of 5% BSA blocking solution was added for 1 hour at room temperature, with shaking. Plates were washed with PBST. Cell lysates were thawed and 100 μl of lysate was added to the ELISA plate. The plates were incubated for 2 hours at room temperature, with shaking. ELISA plates were then washed with PBST and 100 μl of an HRP-labeled detection antibody that binds phosphorylated tyrosine (R&D Systems, cat #841913) was added to each well. Plates were incubated for 1 hour at room temperature, with shaking. Plates were then washed with PBST, and 100 μl TMB substrate solution (R&D Systems, cat #DY999) was added. Plate were incubated in the dark for 20 minutes at room temperature. 50 μl of Stop solution (R&D Systems, cat #DY994) (50 μl) was added to each well and mixed.

Optical density at 450 nm was read on an EnSpire plate reader (Perkin Elmer). The remaining kinase activity by calculated using the following formula: % Relative activity=100×(A450ₛₐₘₚₗₑ−A450_{LC})/(A450_{HC}−A450_{LC}). The low and high control values (“LC” and “HC”) were generated from lysate from wells without cells or with cells treated with 0.1% DMSO, respectively. IC₅₀ values were calculated using XLFit software using a nonlinear regression model with a sigmoidal dose response, as shown in Table B6 below.

TABLE B6

| | pERBB2 IC₅₀ |
|---|---|
| Compound | (nM) |
| Tucatinib | 23.1 |
| Neratinib | 35.6 |
| 1 | 7.94 |
| 2 | 75.9 |
| 4 | 5.17 |
| 7 | 109 |
| 13 | 12.5 |
| 14 | 26.9 |
| 15 | 8.15 |
| 16 | 9.15 |
| 17 | 19.7 |
| 18 | 7.52 |
| 19 | 25.2 |
| 20 | 12 |
| Example S21, | 20.8 |
| Enantiomer # 1 | |
| Example S21, | 22.3 |
| Enantiomer # 2 | |
| 23 | 23.2 |
| 24 | 383 |
| 25 | 1190 |
| 26 | 11.7 |
| 27 | 17.1 |
| 28 | 7.77 |
| 29 | 5.86 |
| 30 | 12.3 |
| 31 | 15.9 |
| 32 | 72.1 |
| 33 | 18.5 |
| Example S33, | 23.2 |
| Entantiomer # 1 | |
| Example S33, | 24.9 |
| Entantiomer # 2 | |
| 36 | 40.9 |
| 37 | 26.9 |
| 38 | 29.3 |
| 39 | 36.2 |
| 40 | 30.7 |
| 41 | 29 |
| Example S40, | 9.9 |
| Enantiomer # 1 | |
| Example S40, | 35.5 |
| Enantiomer # 2 | |
| 44 | 26.2 |
| 45 | 22.6 |
| 46 | 65.1 |
| 47 | 28.9 |
| 48 | 12.4 |
| 49 | 13.9 |
| 50 | 80.1 |
| 51 | 59.5 |
| 52 | 81.2 |
| 53 | 35.6 |
| 54 | 42 |
| 55 | 23.1 |
| 56 | 6520 |
| Example S111, | 16.6 |
| Enantiomer #1 | |
| Example S111, | 18.2 |
| Enantiomer #2 | |
| 59 | 60.7 |
| 60 | 12 |
| 61 | 132 |
| 62 | 23.9 |
| Example S58, | 29.7 |
| Enantiomer # 1 | |
| Example S58, | 44.2 |
| Enantiomer # 2 | |
| 65 | 128 |
| 66 | 34.2 |
| 67 | 44.2 |
| 68 | 30.5 |
| 69 | 56.4 |
| 70 | 34.4 |
| 71 | 25.7 |
| 72 | 44.9 |
| 73 | 41.5 |
| 74 | 5.43 |
| 75 | 20.4 |
| 76 | >10.0E+03 |
| 77 | 18.4 |
| 78 | 16.8 |
| 79 | 8.77 |
| 80 | 66.1 |
| 81 | 72.3 |
| 82 | 54.1 |
| 83 | 44.2 |
| 84 | 425 |
| Example S79, | 36.6 |
| Enantiomer #1 | |
| Example S79, | 15.3 |
| Enantiomer #2 | |
| 87 | 87.2 |
| 88 | 71.5 |
| 89 | 28.9 |
| 90 | 17.1 |
| 91 | 18.9 |
| 92 | 21.3 |
| Example S86, | 11.5 |
| Enantiomer #1 | |
| Example S86, | 138 |
| Enantiomer #2 | |
| 95 | 12.2 |
| 96 | 22.8 |
| 98 | 40.3 |
| 99 | 38.8 |
| 100 | 57.7 |
| 101 | 22.3 |
| 102 | 8.26 |
| Example S108, | 19.1 |
| Enantiomer #1 | |
| Example S108, | 12.7 |
| Enantiomer #2 | |
| 105 | 43.7 |
| 106 | 28.2 |
| 107 | 21.6 |
| 108 | 13.2 |
| 113 | 327 |
| 114 | 31.1 |
| 117 | 19.2 |
| 118 | 10.3 |
| 119 | 15.4 |
| Example S97, | 41.3 |
| Enantiomer #1 | |
| Example S97, | 21 |
| Enantiomer #2 | |
| Example S98, | 11.9 |
| Enantiomer #1 | |
| Example S98, | 26.4 |
| Enantiomer #2 | |
| 124 | 163 |
| 125 | 42.8 |
| 126 | 99.1 |
| 127 | 27 |
| 128 | 21.4 |
| 129 | 11.9 |
| 130 | 40.6 |
| Example S115, | 32 |
| Enantiomer #1 | |
| Example S115, | 273 |
| Enantiomer #2 | |
| 133 | 10.9 |
| Example S117, | 40 |
| Enantiomer #1 | |
| Example S117, | 589 |
| Enantiomer #2 | |
| 136 | 72.6 |
| 137 | 4110 |
| 138 | 16.8 |
| 139 | 14.3 |
| 140 | 20.5 |
| 141 | 23.8 |
| 142 | 15.4 |
| Example S125, | 710 |
| Enantiomer #1 | |
| Example S125, | 76 |
| Enantiomer #2 | |
| Example S126, | 40.9 |
| Enantiomer #1 | |
| Example S126, | 401 |
| Enantiomer #2 | |
| Example S127, | 14 |
| Enantiomer #1 | |
| Example S127, | 146 |
| Enantiomer #2 | |
| 155 | 17.9 |
| 156 | 21.7 |

Enzyme-linked immunosorbent assays (ELISA) were performed to measure phosphorylated EGFR levels using A431 cells (10% FBS). A431 (1.0*10⁴ cells/40 μl/well) cells were seeded in 384 well. Compounds were dissolved in DMSO, serially diluted in DMSO and then were added, mixed, and incubated for 4 hours at 37° C., 5% CO₂. Following the 4-hours incubation, cells were stimulated for 10 minutes with EGF (Invitrogen, cat #PHG0311) at a final concentration of 30 ng/mL in the incubator. The media was aspirated and cells were lysed in 10 μL lysis buffer with protease and phosphatase inhibitors (PerkinElmer, cat #ALSU-PEGFR-A50K). The plates were placed on a shaker for 5 minutes and then incubated for 30 min at 4° C. for complete lysis. The lysate was transferred to an Optiplate (Perkin Elmer, cat #6007290).

Acceptor mix (PerkinElmer, cat #ALSU-PEGFR-A50K) was prepared just before use and 5 μL was dispensed to all the wells, followed by a 1.5-2 h incubation at room temperature in dark. The donor mix (PerkinElmer, cat #ALSU-PEGFR-A50K) was prepared under low light conditions prior to use and 50 of donor mix was added to all the wells under subdued lighting or green filters. The plates were placed on a shaker for 5 min, sealed, and incubated overnight at room temperature in dark. Plates were read on the Envision (PerkinElmer) using standard AlphaLISA settings.

The percentage of inhibition on EGFR phosphorylation was calculated following equation: % Inhibition=100×(LumHC−LumSample)/(LumHC−LumLC). The low and high controls (LC/HC) are generated from lysate from wells with cells treated with DMSO or 10 mM Staurosporine (BioAustralis, cat #BIA-S1086), respectively. IC50 values were calculated by fitting the Curve using XLfit (v5.3.1.3), equation 201: Y=Bottom+(Top−Bottom)/(1+10\hat{ }((Log IC50−X)*HillSlope)). The IC₅₀ values are shown in Table B7 below.

TABLE B7

| Compound | pEGFR IC₅₀ (nM) |
|---|---|
| 1 | 1230 |
| 2 | >10.0E+03 |
| 4 | 205 |
| 13 | 571 |
| 14 | 3800 |
| 15 | 1250 |
| 16 | 235 |
| 17 | >10.0E+03 |
| 18 | >9.64E+03 |
| 19 | 5630 |
| 20 | 586 |
| Example S21, | 538 |
| Enantiomer #1 | |
| Example S21, | 1980 |
| Enantiomer #2 | |
| 23 | 6150 |
| 24 | >10.0E+03 |
| 25 | >10.0E+03 |
| 26 | 1460 |
| 27 | 604 |
| 28 | 355 |
| 29 | 405 |
| 30 | 68.4 |
| 31 | 117 |
| 32 | 8180 |
| 33 | 1140 |
| Example S33, | 1090 |
| Entantiomer # 1 | |
| Example S33, | 520 |
| Entantiomer # 2 | |
| 36 | 2330 |
| 37 | 1020 |
| 38 | 407 |
| 39 | 497 |
| 40 | >10.0E+03 |
| 41 | 4490 |
| Example S40, | 103 |
| Enantiomer # 1 | |
| Example S40, | 6450 |
| Enantiomer # 2 | |
| 44 | 1940 |
| 45 | 670 |
| 46 | 759 |
| 47 | 2220 |
| 48 | 63.5 |
| 49 | 173 |
| 50 | 2150 |
| 51 | 4200 |
| 52 | >10.0E+03 |
| 53 | 76 |
| 54 | 1540 |
| 55 | 250 |
| 56 | >10.0E+03 |
| Example S111, | 274 |
| Enantiomer #1 | |
| Example S111, | 33.7 |
| Enantiomer #2 | |
| 59 | 710 |
| 60 | 3040 |
| 61 | 1010 |
| 62 | 212 |
| Example S58, | 264 |
| Enantiomer # 1 | |
| Example S58, | >10.0E+03 |
| Enantiomer # 2 | |
| 65 | >10.0E+03 |
| 66 | 93.6 |
| 67 | 53.2 |
| 68 | 973 |
| 69 | >10.0E+03 |
| 70 | 1990 |
| 71 | 273 |
| 72 | 1880 |
| 73 | 514 |
| 74 | 2970 |
| 75 | 697 |
| 76 | >10.0E+03 |
| 77 | 7490 |
| 78 | 1570 |
| 79 | >10.0E+03 |
| 80 | 704 |
| 81 | 1130 |
| 82 | 3990 |
| 83 | 6030 |
| 84 | >10.0E+03 |
| Example S79, | 6100 |
| Enantiomer #1 | |
| Example S79, | 798 |
| Enantiomer #2 | |
| 87 | 8160 |
| 88 | >10.0E+03 |
| 89 | 699 |
| 90 | 5770 |
| 91 | >10.0E+03 |
| 92 | >10.0E+03 |
| Example S86, | 574 |
| Enantiomer #1 | |
| Example S86, | >10.0E+03 |
| Enantiomer #2 | |
| 95 | 476 |
| 96 | 794 |
| 98 | 942 |
| 99 | >10.0E+03 |
| 100 | 157 |
| 101 | 1160 |
| 102 | 3010 |
| Example S108, | 6680 |
| Enantiomer #1 | |
| Example S108, | 175 |
| Enantiomer #2 | |
| 105 | 503 |
| 106 | 1690 |
| 107 | >10.0E+03 |
| 108 | 3410 |
| 113 | >10.0E+03 |
| 114 | >10.0E+03 |
| 117 | >10.0E+03 |
| 118 | 177 |
| 119 | 521 |
| Example S97, | >10.0E+03 |
| Enantiomer #1 | |
| Example S97, | 490 |
| Enantiomer #2 | |
| Example S98, | 158 |
| Enantiomer #1 | |
| Example S98, | >10.0E+03 |
| Enantiomer #2 | |
| 124 | >10.0E+03 |
| 125 | 1140 |
| 126 | 1110 |
| 127 | 1200 |
| 128 | >10.0E+03 |
| 129 | 2320 |
| 130 | 187 |
| Example S115, | 1000 |
| Enantiomer #1 | |
| Example S115, | >10.0E+03 |
| Enantiomer #2 | |
| 133 | 1140 |
| Example S117, | 968 |
| Enantiomer #1 | |
| Example S117, | >10.0E+03 |
| Enantiomer #2 | |
| 136 | 1670 |
| 137 | >10.0E+03 |
| 138 | >10.0E+03 |
| 139 | >10.0E+03 |
| 140 | >10.0E+03 |
| 141 | >10.0E+03 |
| 142 | 543 |
| Example S125, | >10,000 |
| Enantiomer #1 | |
| Example S125, | 2,300 |
| Enantiomer #2 | |
| Example S126, | 299 |
| Enantiomer #1 | |
| Example S126, | >10,000 |
| Enantiomer #2 | |
| Example S127, | 575 |
| Enantiomer #1 | |
| Example S127, | >10.0E+03 |
| Enantiomer #2 | |
| 155 | 331 |
| 156 | 7760 |

What is claimed is:

1. A compound of formula (I) 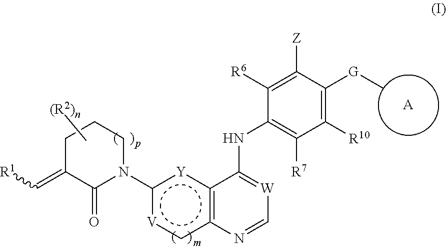
or a pharmaceutically acceptable salt, solvate, hydrate, or co-crystal thereof, or a mixture of any of the foregoing, wherein:
ring A is 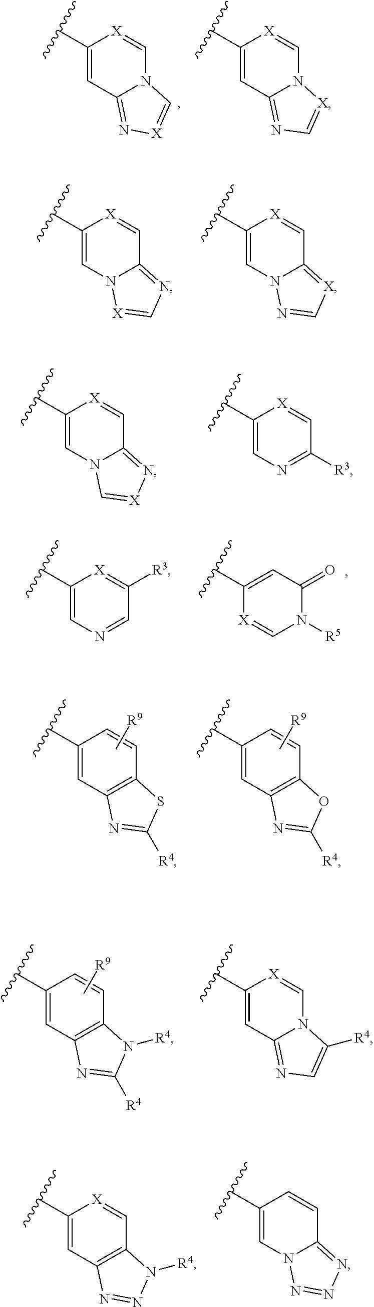 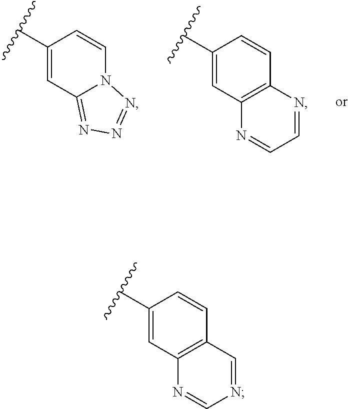 [SMILES: *c1ccn2nnnc2c1 ; *c1ccc2nccnc2c1 ; "or" ; *c1ccc2cncnc2c1]
V is N, S, or C—R⁸;
W is N or C—CN;
each X is independently N or CH;
G is —CR^{g1}R^{g2}—, —O—, or —S—, wherein R^{g1} and R^{g2} are independently —H or —F;
Y is N or C—R^{y}, wherein R^{y} is —H or —F;
Z is —H, halogen, —OCH₃, —C≡CH, or C₁-C₂ alkyl;
R¹ is —H, C₁-C₃ alkyl, or 3- to 7-membered heterocycloalkyl,
wherein the C₁-C₃ alkyl is optionally substituted by 1-4 substituents selected from the group consisting of —F, —OH, a 3- to 7-membered carbon-linked N-heterocycloalkyl, and —NR^{1a}R^{1b},
wherein each R^{1a} and R^{1b} is independently hydrogen, —CD₃, or C₁-C₃ alkyl, or wherein each pair of geminal R^{1a} and R^{1b} may be taken together with the nitrogen atom to which they are attached to form an N-heterocycloalkyl,
wherein the 3- to 7-membered heterocycloalkyl of R¹ is optionally substituted by —F, and
wherein each heterocyclic nitrogen atom, if present, is independently optionally substituted with C₁-C₃ alkyl, and wherein R¹ may be cis or trans when R¹ is not —H;
m is an integer 0 or 1, wherein if m=0, then V is S, and if m=1, then V is N or CR⁸;
n is an integer 0, 1, or 2;
p is an integer 0 or 1;
each R² is independently —H or C₁-C₃ alkyl, wherein the C₁-C₃ alkyl is optionally substituted by 1-4 substituents selected from the group consisting of —F, —OH, —O(C₁-C₃ alkyl), a 3- to 7-membered carbon-linked N-heterocycloalkyl, and NR^{1a}R^{1b} wherein the C₁-C₃ alkyl of the —O(C₁-C₃ alkyl) is optionally substituted by —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl; or
two R² groups on the same carbon atom may be taken together with the carbon atom to which they are attached to form a C₃-C₆ spirocycloalkyl or 4- to 6-membered spiroheterocycloalkyl containing 1-2 ring heteroatoms selected from the group consisting of N, O, and S; or
two R² groups on vicinal carbon atoms may be taken together with the two carbon atoms to form a fused C₃-C₆ cycloalkyl;
R³ is —H, —F, —CD₃, C₁-C₃ alkyl, —CF₂H, —CN, —OR⁴, —SR⁴, —S(O)(C₁-C₃ alkyl), or —S(O)₂(C₁-C₃ alkyl);
each R⁴ is independently —H, —CD₃, C₁-C₃ alkyl, —CF₂H, —CF₃, or cyclopropyl;
R⁵ is C₁-C₃ alkyl, —CD₃, —CF₂H, allyl, —CH₂-cyclopropyl, cyclopropyl, or —OR⁴;
R⁶ is —H, -halogen, or C₁-C₃ alkyl;
R⁷ is —H, -halogen, or C₁-C₃ alkyl;
R⁸ is —H, —F, C₁-C₃ alkyl, or —O(C₁-C₃ alkyl),
wherein the C₁-C₃ alkyl and the C₁-C₃ alkyl of the —O(C₁-C₃ alkyl) are each optionally substituted by 1-4 substituents selected from the group consisting of —F, —OH, —OR^{8a}, and —NR^{8a}R^{8b}, wherein each R^{8a} and R^{8b} are independently H or C₁-C₃ alkyl, or wherein each pair of geminal R^{8a} and R^{8b} may be taken together with the nitrogen atom to which they are attached to form an N-heterocycloalkyl wherein the N-heterocycloalkyl is optionally substituted by C₁-C₃ alkyl; and
R⁹ is —H, halogen, —CN, C₁-C₃ alkyl, —CF₂H, —CF₃, cyclopropyl, —O(C₁-C₃ alkyl), or O-cyclopropyl; and
R¹⁰ is —H or halogen.

2. The compound of claim 1, or a pharmaceutically acceptable salt, solvate, hydrate, or co-crystal thereof, or a mixture of any of the foregoing, wherein p is 0.

3. The compound of claim 1, or a pharmaceutically acceptable salt, solvate, hydrate, or co-crystal thereof, or a mixture of any of the foregoing, wherein p is 1.

4. The compound of any one of claims 1-3, or a pharmaceutically acceptable salt, solvate, hydrate, or co-crystal thereof, or a mixture of any of the foregoing, wherein m is 0.

5. The compound of any one of claims 1-3, or a pharmaceutically acceptable salt, solvate, hydrate, or co-crystal thereof, or a mixture of any of the foregoing, wherein m is 1.

6. The compound of claim 1, wherein the compound of formula (I) is a compound of formula (II-a) 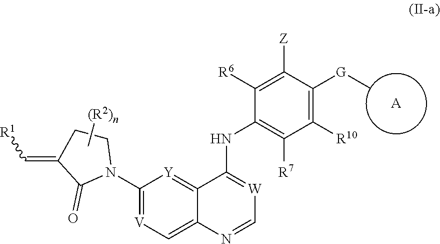
or a pharmaceutically acceptable salt, solvate, hydrate, or co-crystal thereof, or a mixture of any of the foregoing.

7. The compound of claim 1, wherein the compound of formula (I) is a compound of formula (II-b) 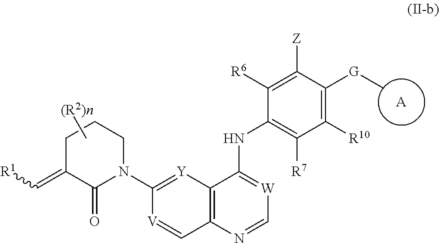
or a pharmaceutically acceptable salt, solvate, hydrate, or co-crystal thereof, or a mixture of any of the foregoing.

8. The compound of claim 1, wherein the compound of formula (I) is a compound of formula (II-c) 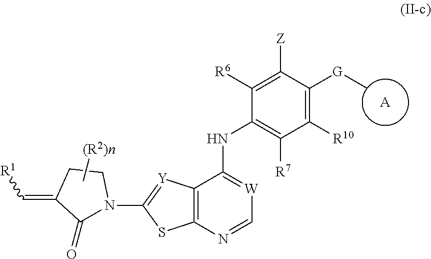
or a pharmaceutically acceptable salt, solvate, hydrate, or co-crystal thereof, or a mixture of any of the foregoing.

9. The compound of claim 1, wherein the compound of formula (I) is a compound of formula (II-d) 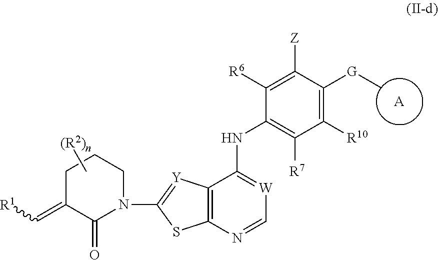
or a pharmaceutically acceptable salt, solvate, hydrate, or co-crystal thereof, or a mixture of any of the foregoing.

10. The compound of any one of claims 1 to 9, wherein Ring A is 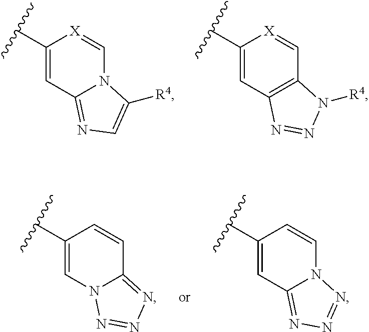
a pharmaceutically acceptable salt, solvate, hydrate, or co-crystal thereof, or a mixture of any of the foregoing.

11. A compound of formula (III) 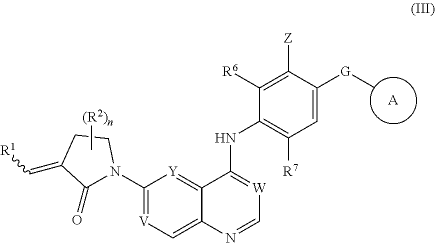
or a pharmaceutically acceptable salt, solvate, hydrate, or co-crystal thereof, or a mixture of any of the foregoing, wherein:
ring A is 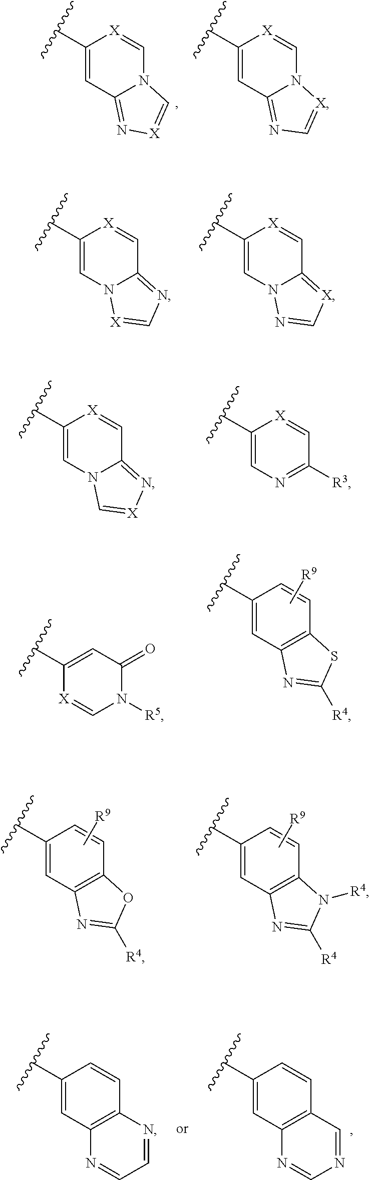
V is N or C—R⁸;
W is N or C—CN;
each X is independently N or CH;
G is —CR^{g1}R^{g2}—, —O—, or —S—, wherein R^{g1} and R^{g2} are independently —H or —F;
Y is N or C—R^{y}, wherein R^{y} is —H or —F;
Z is —H, —F, —Cl, or C₁-C₂ alkyl;
R¹ is —H or C₁-C₃ alkyl,
wherein the C₁-C₃ alkyl is optionally substituted by 1-4 substituents selected from the group consisting of —F, —OH, a 3- to 7-membered carbon-linked N-heterocycloalkyl, and —NR^{1a}R^{1b}, wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl, and wherein R¹ may be cis or trans when R¹ is not —H;
n is an integer 0, 1, or 2;
each R² is independently —H or C₁-C₃ alkyl, wherein the C₁-C₃ alkyl is optionally substituted by 1-4 substituents selected from the group consisting of —F, —OH, —O(C₁-C₃ alkyl), a 3- to 7-membered carbon-linked N-heterocycloalkyl, and NR^{1a}R^{1b} wherein the C₁-C₃ alkyl of the —O(C₁-C₃ alkyl) is optionally substituted by —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl; or
two R² groups on the same carbon atom may be taken together with the carbon atom to which they are attached to form a C₃-C₆ spirocycloalkyl or 4- to 6-membered spiroheterocycloalkyl containing 1-2 ring heteroatoms selected from the group consisting of N, O, and S; or
two R² groups on vicinal carbon atoms may be taken together with the two carbon atoms to form a fused C₃-C₆ cycloalkyl;
R³ is —H, —CD₃, C₁-C₃ alkyl, —CF₂H, —CN, —OR⁴, —SR⁴, —S(O)(C₁-C₃ alkyl), or —S(O)₂(C₁-C₃ alkyl);
each R⁴ is independently —H, C₁-C₃ alkyl, —CF₂H, —CF₃, or cyclopropyl;
R⁵ is C₁-C₃ alkyl, —CD₃, —CF₂H, allyl, —CH₂-cyclopropyl, cyclopropyl, or —OW;
R⁶ is —H or —F;
R⁷ is —H or —F;
R⁸ is —H, —F, or —O(C₁-C₃ alkyl), wherein the C₁-C₃ alkyl is optionally substituted by 1-4 substituents selected from the group consisting of —F, —OH, —OR^{8a}, and —NR^{8a}R^{8b}, wherein each R^{8a} and R^{8b} are independently H or C₁-C₃ alkyl, or wherein each pair of geminal R^{8a} and R^{8b} may be taken together with the nitrogen atom to which they are attached to form an N-heterocycloalkyl wherein the N-heterocycloalkyl is optionally substituted by C₁-C₃ alkyl; and
R⁹ is —H, halogen, —CN, C₁-C₃ alkyl, —CF₂H, —CF₃, cyclopropyl, —O(C₁-C₃ alkyl), or O-cyclopropyl.

12. The compound of any one of claims 1 to 9 and 11, or a pharmaceutically acceptable salt, solvate, hydrate, or co-crystal thereof, or a mixture of any of the foregoing, wherein Ring A is

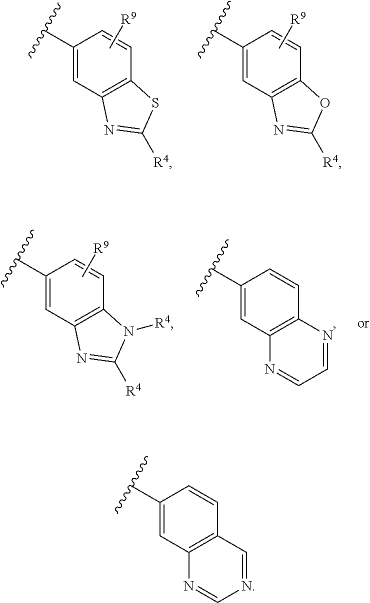

13. The compound of any one of claims 1 to 9 and 11 to 12, or a pharmaceutically acceptable salt, solvate, hydrate, or co-crystal thereof, or a mixture of any of the foregoing, wherein Ring A is

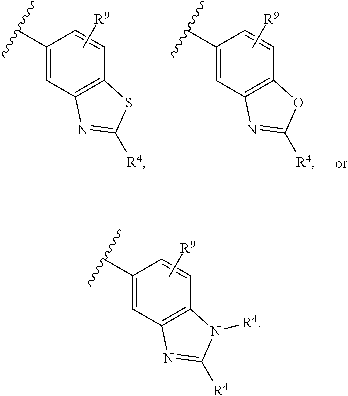

14. The compound of any one of claims 1 to 9 and 11 to 13, or a pharmaceutically acceptable salt, solvate, hydrate, or co-crystal thereof, or a mixture of any of the foregoing, wherein Ring A is

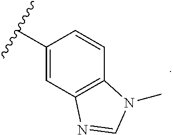 [SMILES: *c1ccc2c(c1)ncn2C]

15. The compound of any one of claims 1 to 7 and 10 to 14, or a pharmaceutically acceptable salt, solvate, hydrate, or co-crystal thereof, or a mixture of any of the foregoing, wherein rO(C₁-C₃ alkyl), wherein the C₁-C₃ alkyl is optionally substituted with —NR^{1a}R^{1b}.

16. A compound of formula (IV) 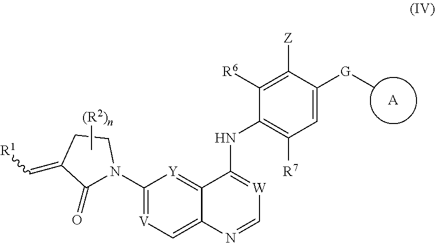
or a pharmaceutically acceptable salt, solvate, hydrate, or co-crystal thereof, or a mixture of any of the foregoing, wherein:
ring A is 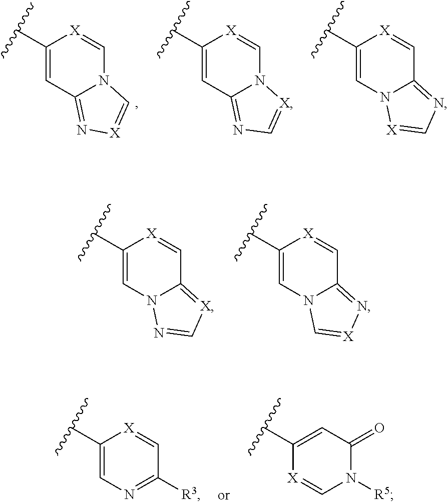
V is N or C—R⁸;
W is N or C—CN;
each X is independently N or CH;
G is —CR^{g1}R^{g2}—, —O—, or —S—, wherein R^{g1} and R^{g2} are independently —H or —F;
Y is N or C—R^{y}, wherein R^{y} is —H or —F;
Z is —H, —F, —Cl, or C₁-C₂ alkyl;
R¹ is —H or C₁-C₃ alkyl,
wherein the C₁-C₃ alkyl is optionally substituted by 1-4 substituents selected from the group consisting of —F, —OH, a 3- to 7-membered carbon-linked N-heterocycloalkyl, and —NR^{1a}R^{1b}, wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl, and wherein R¹ may be cis or trans when R¹ is not —H;
n is an integer 0, 1, or 2;
each R² is independently —H or C₁-C₃ alkyl, wherein the C₁-C₃ alkyl is optionally substituted by 1-4 substituents selected from the group consisting of —F, —OH, —O(C₁-C₃ alkyl), a 3- to 7-membered carbon-linked N-heterocycloalkyl, and —NR^{1a}R^{1b} wherein the C₁-C₃ alkyl of the —O(C₁-C₃ alkyl) is optionally substituted by —NR^{1a}R^{1b} wherein each R^{1a} and R^{1b} are independently C₁-C₃ alkyl or wherein each pair of geminal R^{1a} and R^{1b} may be taken together with the nitrogen atom to which they are attached to form an N-heterocycloalkyl; or
two R² groups on the same carbon atom may be taken together with the carbon atom to which they are attached to form a C₃-C₆ spirocycloalkyl or 4- to 6-membered spiroheterocycloalkyl containing 1-2 ring heteroatoms selected from the group consisting of N, O, and S; or
two R² groups on vicinal carbon atoms may be taken together with the two carbon atoms to form a fused C₃-C₆ cycloalkyl;
R³ is —H, —CD₃, C₁-C₃ alkyl, —CF₂H, —CN, —OR⁴, —SR⁴, —S(O)(C₁-C₃ alkyl), or —S(O)₂(C₁-C₃ alkyl);
each R⁴ is independently —H, C₁-C₃ alkyl, —CF₂H, —CF₃, or cyclopropyl;
R⁵ is C₁-C₃ alkyl, —CD₃, —CF₂H, allyl, —CH₂-cyclopropyl, cyclopropyl, or —OW;
R⁶ is —H or —F;
R⁷ is —H or —F, and
R⁸ is —H or —F.

17. The compound of any one of claims 1 to 9, 11, 15, and 16, or a pharmaceutically acceptable salt, solvate, hydrate, or co-crystal thereof, or a mixture of any of the foregoing, wherein:
ring A is

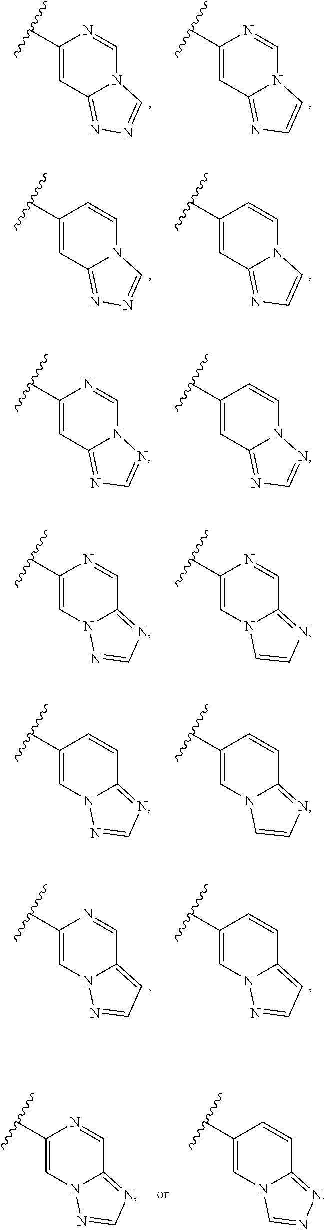

18. The compound of claim 17, or a pharmaceutically acceptable salt, solvate, hydrate, or co-crystal thereof, or a mixture of any of the foregoing, wherein: ring A is

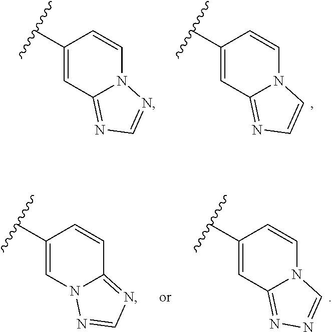 [SMILES: *c1ccn2ncnc2c1 ; *c1ccn2ccnc2c1 ; *c1ccc2ncnn2c1 ; *c1ccn2cnnc2c1]

19. The compound of claim 18, or a pharmaceutically acceptable salt, solvate, hydrate, or co-crystal thereof, or a mixture of any of the foregoing, wherein:
ring A is

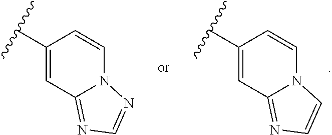 [SMILES: *c1ccn2ncnc2c1 ; *c1ccn2ccnc2c1]

20. The compound of any one of claims 1 to 9, 11, 15 and 16, or a pharmaceutically acceptable salt, solvate, hydrate, or co-crystal thereof, or a mixture of any of the foregoing, wherein:
ring A is

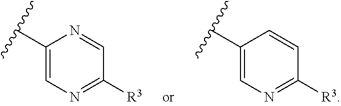

21. The compound of claim 20, or a pharmaceutically acceptable salt, solvate, hydrate, or co-crystal thereof, or a mixture of any of the foregoing, wherein R³ is —H, —CD₃, C₁-C₂ alkyl, or —CF₂H.

22. The compound of claim 21, or a pharmaceutically acceptable salt, solvate, hydrate, or co-crystal thereof, or a mixture of any of the foregoing, wherein R³ is —CH₃ or —CH₂CH₃.

23. The compound of any one of claims 1 to 9, 11, 15 and 16, or a pharmaceutically acceptable salt, solvate, hydrate, or co-crystal thereof, or a mixture of any of the foregoing, wherein:
ring A is

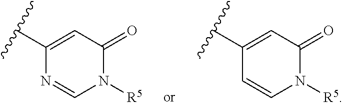

24. The compound of claim 23, or a pharmaceutically acceptable salt, solvate,
hydrate, or co-crystal thereof, or a mixture of any of the foregoing, wherein R⁵ is C₁-C₂ alkyl, —CD₃, or —CF₂H.

25. The compound of claim 24, or a pharmaceutically acceptable salt, solvate, hydrate, or co-crystal thereof, or a mixture of any of the foregoing, wherein R⁵ is —CH₃ or —CF₂H.

26. The compound of any one of claims 1 to 9, 11, 15 and 16, or a pharmaceutically acceptable salt, solvate, hydrate, or co-crystal thereof, or a mixture of any of the foregoing, wherein:
ring A is 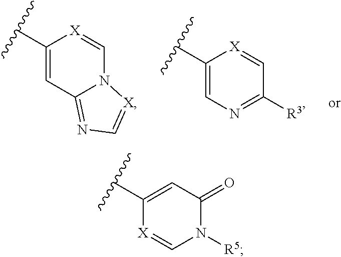
R³ is H, —CD₃, C₁-C₂ alkyl, or —CF₂H, and
R⁵ is C₁-C₂ alkyl, —CD₃, or —CF₂H.

27. The compound of claim 26, or a pharmaceutically acceptable salt, solvate, hydrate, or co-crystal thereof, or a mixture of any of the foregoing, wherein:
R³ is —CH₃ or —CF₂H, and
R⁵ is —CH₃ or —CF₂H.

28. The compound of any one of claims 1-27, or a pharmaceutically acceptable salt, solvate, hydrate, or co-crystal thereof, or a mixture of any of the foregoing, wherein Z is —H, —F, —Cl, or —CH₃.

29. The compound of any one of claims 1-28, or a pharmaceutically acceptable salt, solvate, hydrate, or co-crystal thereof, or a mixture of any of the foregoing, wherein Z is —CH₃.

30. The compound of any one of claims 1-29, or a pharmaceutically acceptable salt, solvate, hydrate, or co-crystal thereof, or a mixture of any of the foregoing, wherein R¹ is —H.

31. The compound of any one of claims 1-29, or a pharmaceutically acceptable salt, solvate, hydrate, or co-crystal thereof, or a mixture of any of the foregoing, wherein R¹ is C₁-C₃ alkyl optionally substituted by —OH or —N(C₁-C₃ alkyl)(C₁-C₃ alkyl).

32. The compound of claim 31, or a pharmaceutically acceptable salt, solvate, hydrate, or co-crystal thereof, or a mixture of any of the foregoing, wherein R¹ is —CH₃, —CH₂OH, (CH₂)₂OH or —CH₂N(CH₃)₂.

33. The compound of claim 31 or 32, or a pharmaceutically acceptable salt, solvate, hydrate, or co-crystal thereof, or a mixture of any of the foregoing, wherein R¹ is —CH₃.

34. The compound of any one of claims 1-33, or a pharmaceutically acceptable salt, solvate, hydrate, or co-crystal thereof, or a mixture of any of the foregoing, wherein Y is N.

35. The compound of any one of claims 1-33, or a pharmaceutically acceptable salt, solvate, hydrate, or co-crystal thereof, or a mixture of any of the foregoing, wherein Y is C—R^{y}.

36. The compound of claim 1-33 and 35, or a pharmaceutically acceptable salt, solvate, hydrate, or co-crystal thereof, or a mixture of any of the foregoing, wherein Y is C—R^{y}, and R^{y} is —H.

37. The compound of claim 1-33 and 35, or a pharmaceutically acceptable salt, solvate, hydrate, or co-crystal thereof, or a mixture of any of the foregoing, wherein Y is C—R^{y}, and R^{y} is —F.

38. The compound of any one of claims 1-37, or a pharmaceutically acceptable salt, solvate, hydrate, or co-crystal thereof, or a mixture of any of the foregoing, wherein W is N.

39. The compound of any one of claims 1-37, or a pharmaceutically acceptable salt, solvate, hydrate, or co-crystal thereof, or a mixture of any of the foregoing, wherein W is C—CN.

40. The compound of any one of claims 1-7, 10-14 and 16-39, or a pharmaceutically acceptable salt, solvate, hydrate, or co-crystal thereof, or a mixture of any of the foregoing, wherein V is N.

41. The compound of any one of claims 1-7 and 10-39, or a pharmaceutically acceptable salt, solvate, hydrate, or co-crystal thereof, or a mixture of any of the foregoing, wherein V is C—R⁸.

42. The compound of any one of claims 1-41, or a pharmaceutically acceptable salt, solvate, hydrate, or co-crystal thereof, or a mixture of any of the foregoing, wherein n is an integer 1 or 2.

43. The compound of any one of claims 1-42, or a pharmaceutically acceptable salt, solvate, hydrate, or co-crystal thereof, or a mixture of any of the foregoing, wherein:
n is 2; and
the two R² groups are present on the same carbon atom and are taken together with the carbon atom to which they are attached to form a C₃-C₆ spirocycloalkyl or 4- to 6-membered spiroheterocycloalkyl containing 1-2 ring heteroatoms selected from the group consisting of N, O, and S, or
the two R² groups are on vicinal carbon atoms and are taken together with the two carbon atoms to form a fused C₃-C₆ cycloalkyl.

44. The compound of any one of claims 1-43, or a pharmaceutically acceptable salt, solvate, hydrate, or co-crystal thereof, or a mixture of any of the foregoing, wherein:
n is 2; and
the two R² groups are present on the same carbon atom and are taken together with the carbon atom to which they are attached to form a C₃-C₆ spirocycloalkyl.

45. The compound of any one of claims 1-43, or a pharmaceutically acceptable salt, solvate, hydrate, or co-crystal thereof, or a mixture of any of the foregoing, wherein:
n is 2; and
the two R² groups are on vicinal carbon atoms and are taken together with the two carbon atoms to form a fused C₃-C₆ cycloalkyl.

46. The compound of any one of claims 1-42, or a pharmaceutically acceptable salt, solvate, hydrate, or co-crystal thereof, or a mixture of any of the foregoing, wherein each R² is independently —H or C₁-C₃ alkyl.

47. The compound of any one of claims 1-46, or a pharmaceutically acceptable salt, solvate, hydrate, or co-crystal thereof, or a mixture of any of the foregoing, wherein G is —O—.

48. The compound of any one of claims 1-46, or a pharmaceutically acceptable salt, solvate, hydrate, or co-crystal thereof, or a mixture of any of the foregoing, wherein G is —CH₂—.

49. The compound of any one of claims 1-46, or a pharmaceutically acceptable salt, solvate, hydrate, or co-crystal thereof, or a mixture of any of the foregoing, wherein G is —S—.

50. The compound of any one of claims 1-49, or a pharmaceutically acceptable salt, solvate, hydrate, or co-crystal thereof, or a mixture of any of the foregoing, wherein R⁶ is —H.

51. The compound of any one of claims 1-49, or a pharmaceutically acceptable salt, solvate, hydrate, or co-crystal thereof, or a mixture of any of the foregoing, wherein R⁶ is —F.

52. The compound of any one of claims 1-51, or a pharmaceutically acceptable salt, solvate, hydrate, or co-crystal thereof, or a mixture of any of the foregoing, wherein R⁷ is —H.

53. The compound of any one of claims 1-51, or a pharmaceutically acceptable salt, solvate, hydrate, or co-crystal thereof, or a mixture of any of the foregoing, wherein R⁷ is —F.

54. A compound selected from the group consisting of: 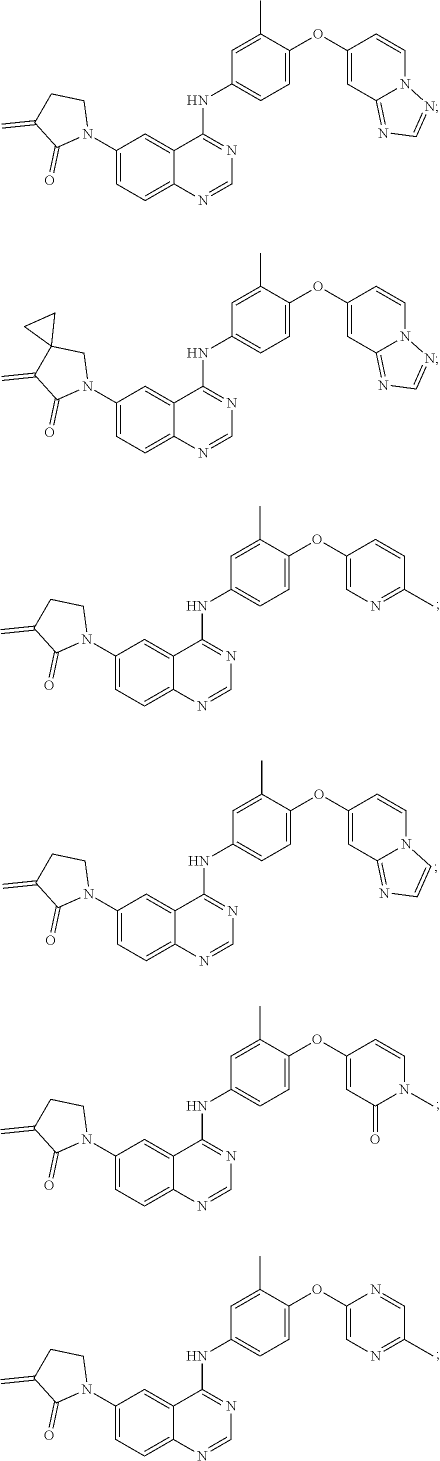 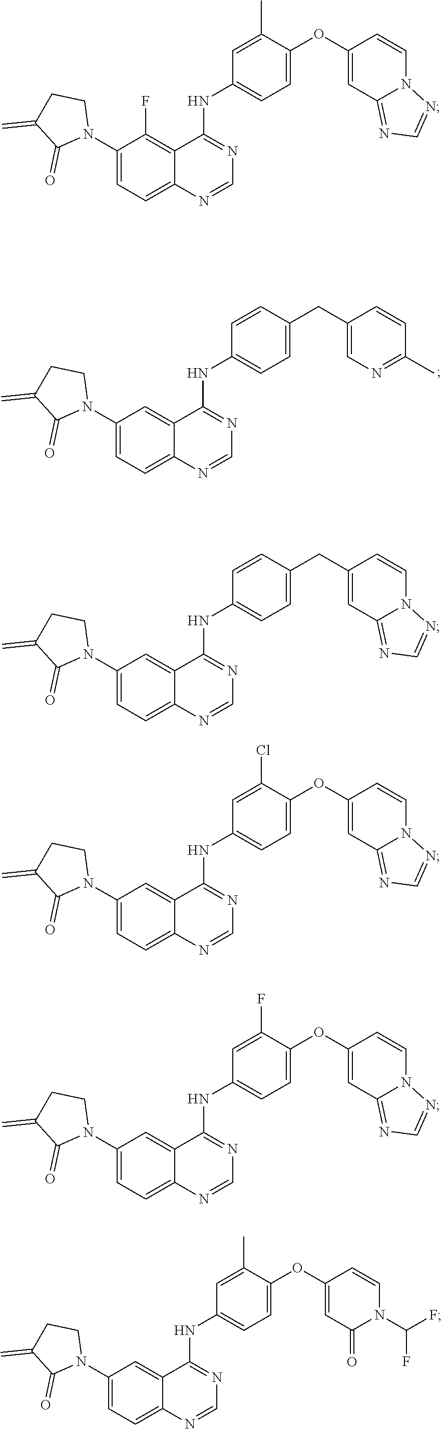 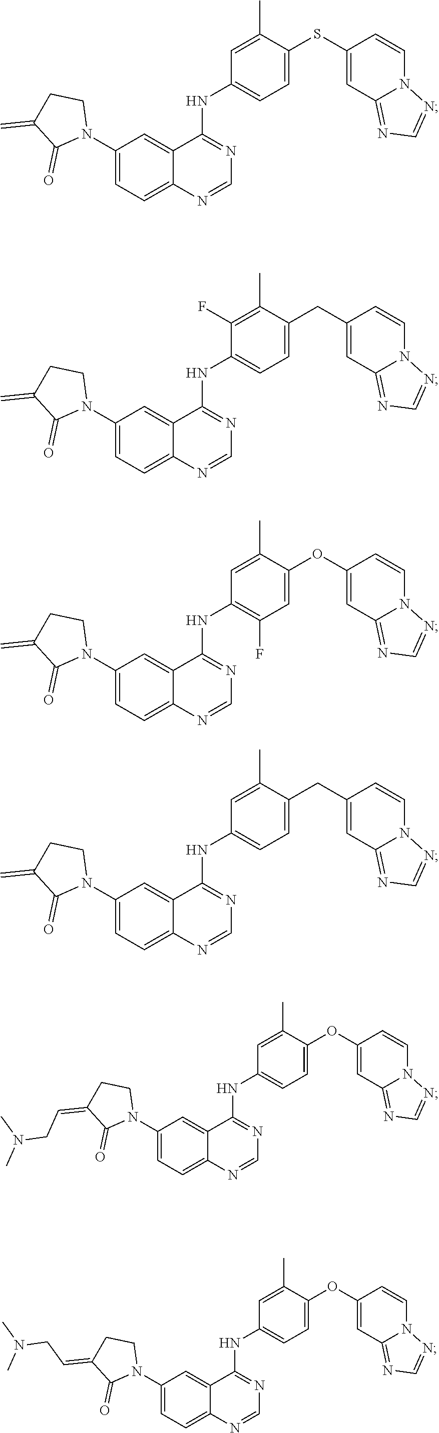 [SMILES: C=C1CCN(c2ccc3ncnc(Nc4ccc(Sc5ccn6ncnc6c5)c(C)c4)c3c2)C1=O ; C=C1CCN(c2ccc3ncnc(Nc4ccc(Cc5ccn6ncnc6c5)c(C)c4F)c3c2)C1=O ; C=C1CCN(c2ccc3ncnc(Nc4cc(C)c(Oc5ccn6ncnc6c5)cc4F)c3c2)C1=O ; C=C1CCN(c2ccc3ncnc(Nc4ccc(Cc5ccn6ncnc6c5)c(C)c4)c3c2)C1=O ; Cc1cc(Nc2ncnc3ccc(N4CC/C(=C/CN(C)C)C4=O)cc23)ccc1Oc1ccn2ncnc2c1 ; Cc1cc(Nc2ncnc3ccc(N4CC/C(=C\CN(C)C)C4=O)cc23)ccc1Oc1ccn2ncnc2c1] 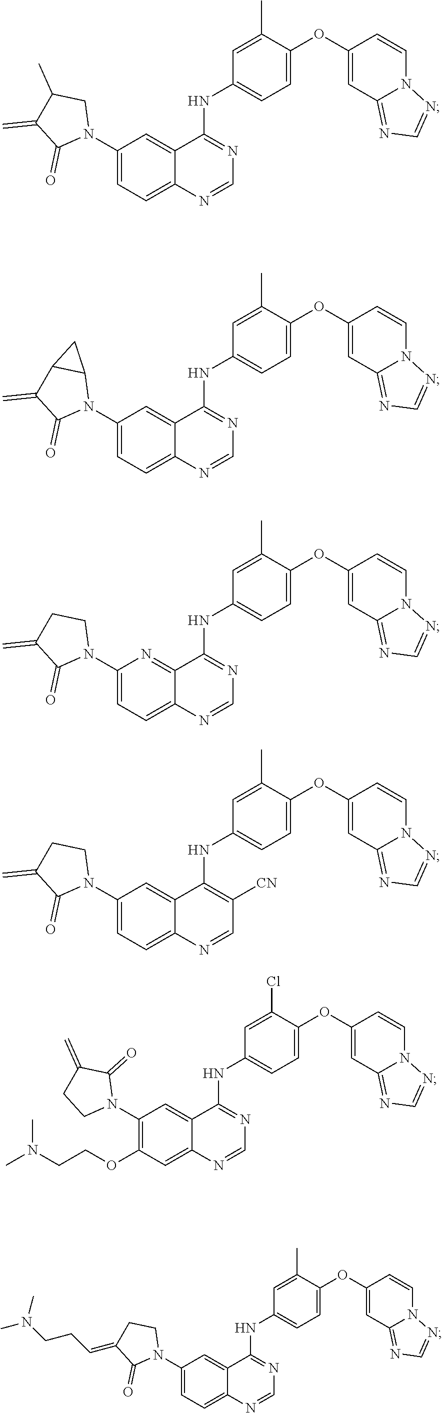 [SMILES: C=C1C(=O)N(c2ccc3ncnc(Nc4ccc(Oc5ccn6ncnc6c5)c(C)c4)c3c2)CC1C ; C=C1C(=O)N(c2ccc3ncnc(Nc4ccc(Oc5ccn6ncnc6c5)c(C)c4)c3c2)C2CC12 ; C=C1CCN(c2ccc3ncnc(Nc4ccc(Oc5ccn6ncnc6c5)c(C)c4)c3n2)C1=O ; C=C1CCN(c2ccc3ncc(C#N)c(Nc4ccc(Oc5ccn6ncnc6c5)c(C)c4)c3c2)C1=O ; C=C1CCN(c2cc3c(Nc4ccc(Oc5ccn6ncnc6c5)c(Cl)c4)ncnc3cc2OCCN(C)C)C1=O ; Cc1cc(Nc2ncnc3ccc(N4CC/C(=C\CCN(C)C)C4=O)cc23)ccc1Oc1ccn2ncnc2c1] 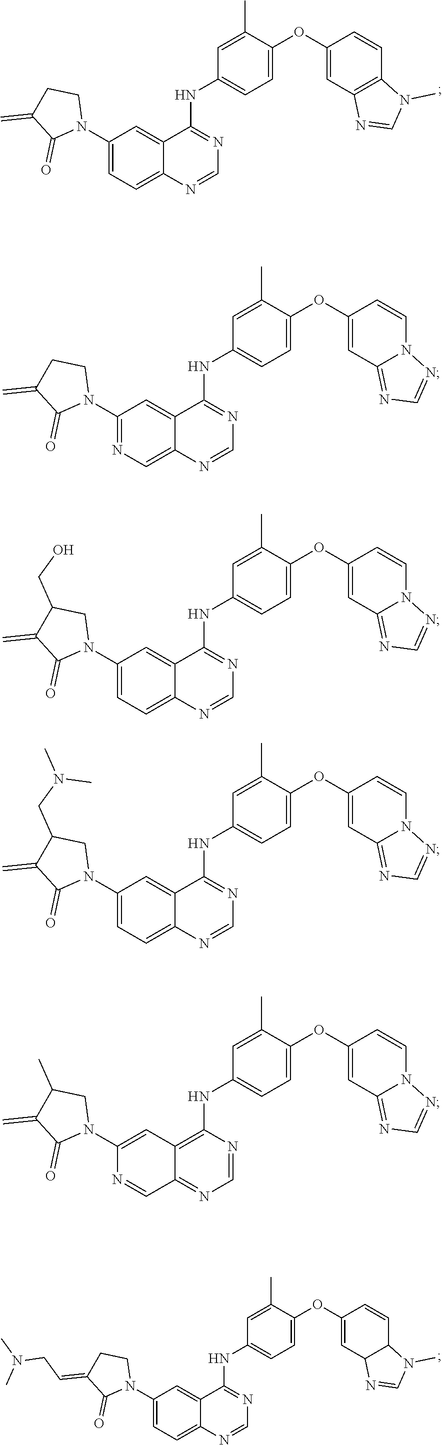 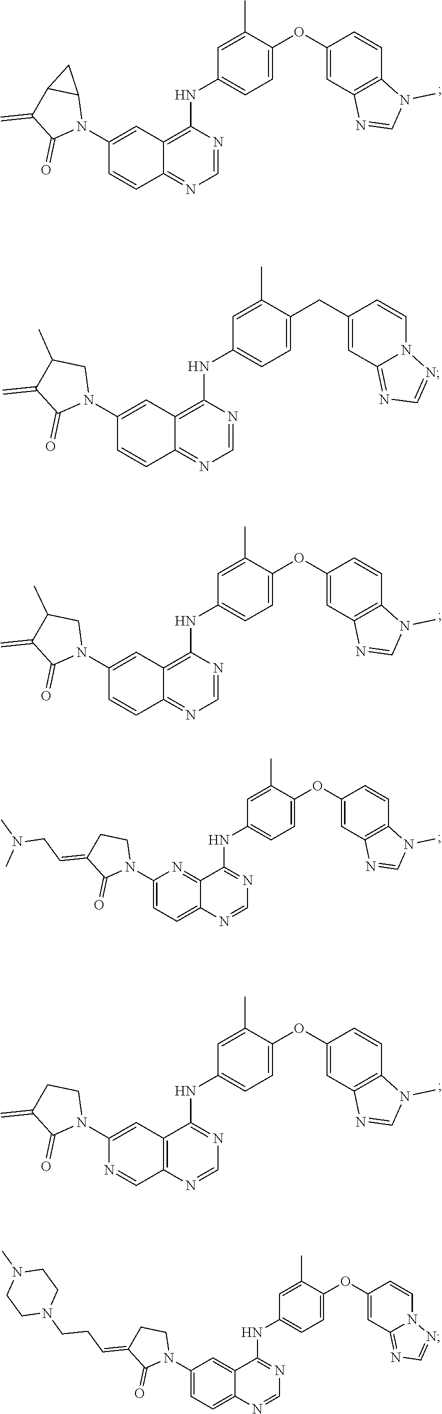 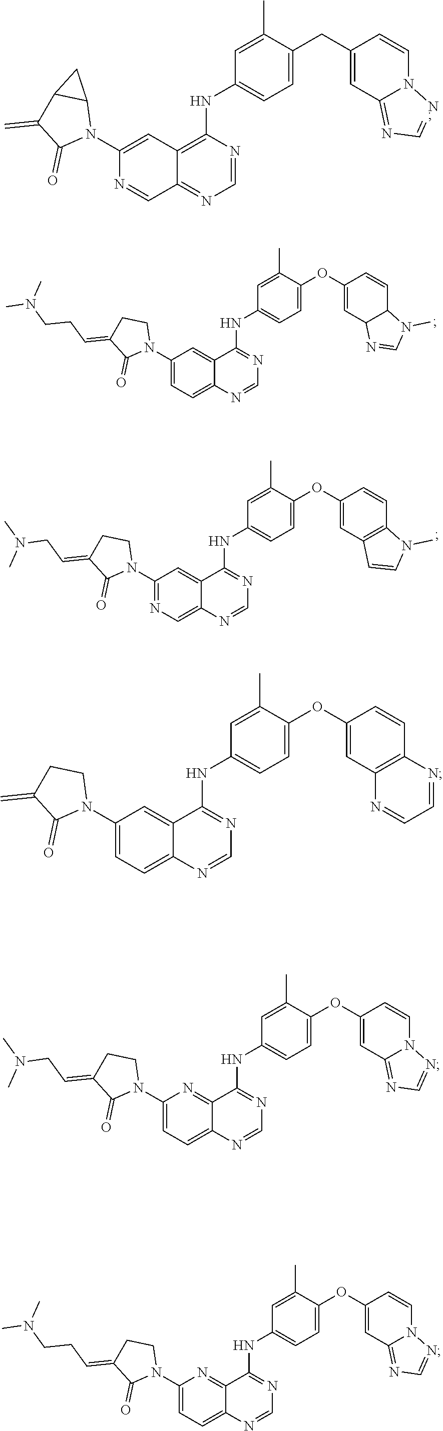 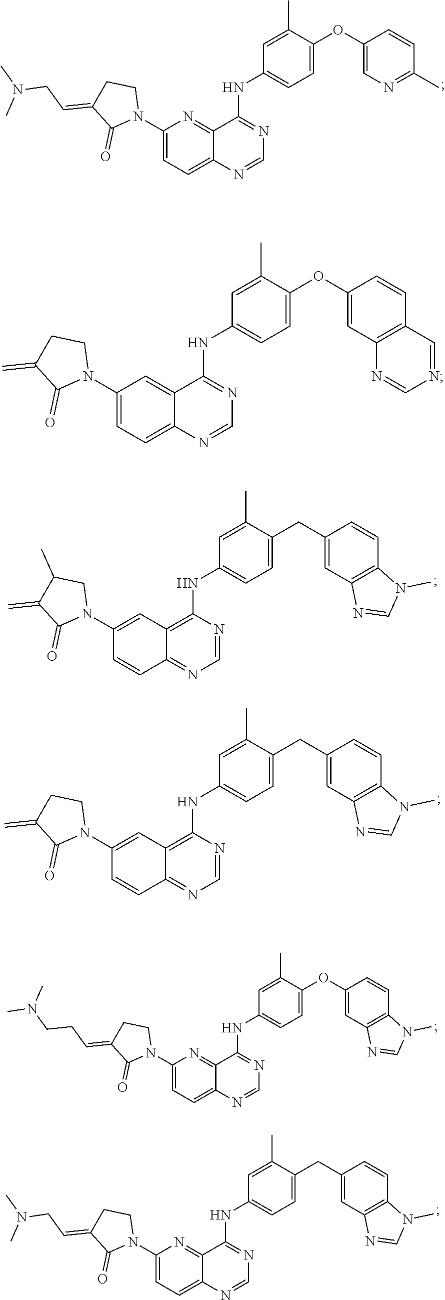 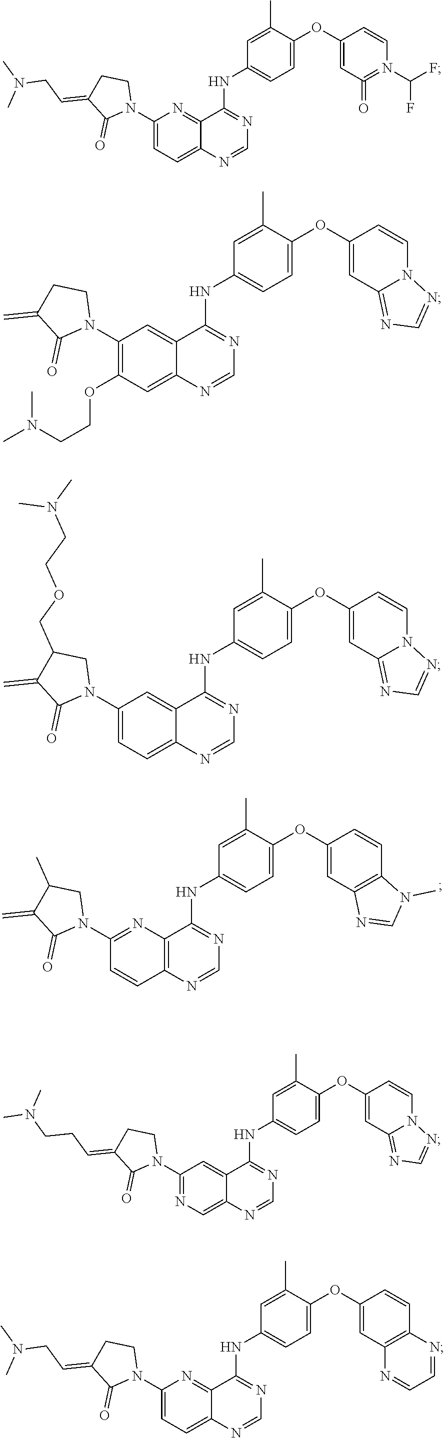 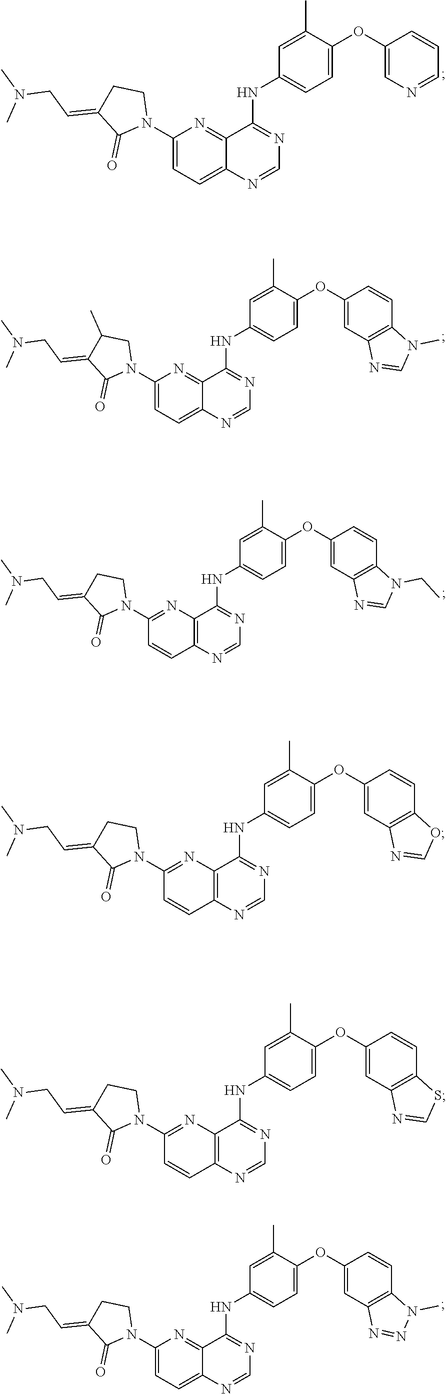 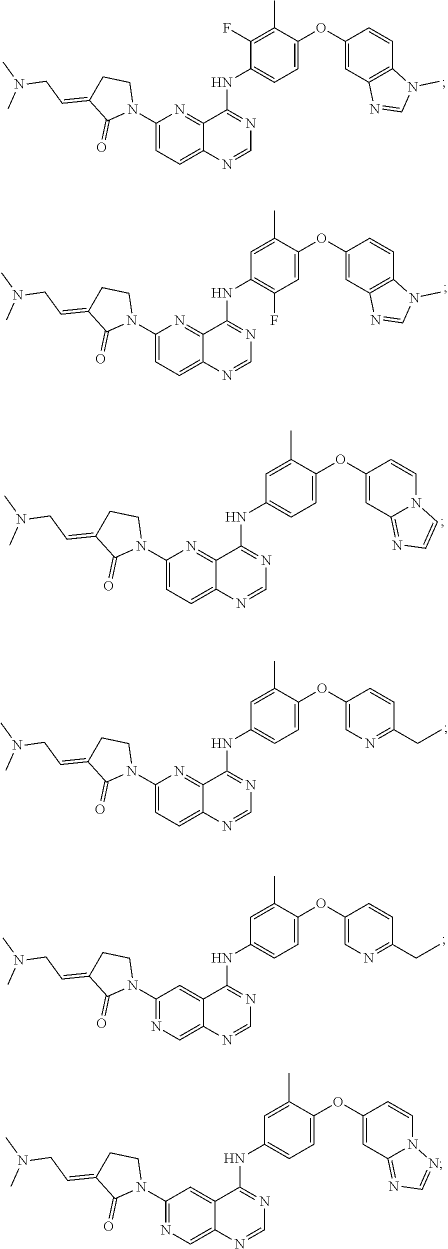 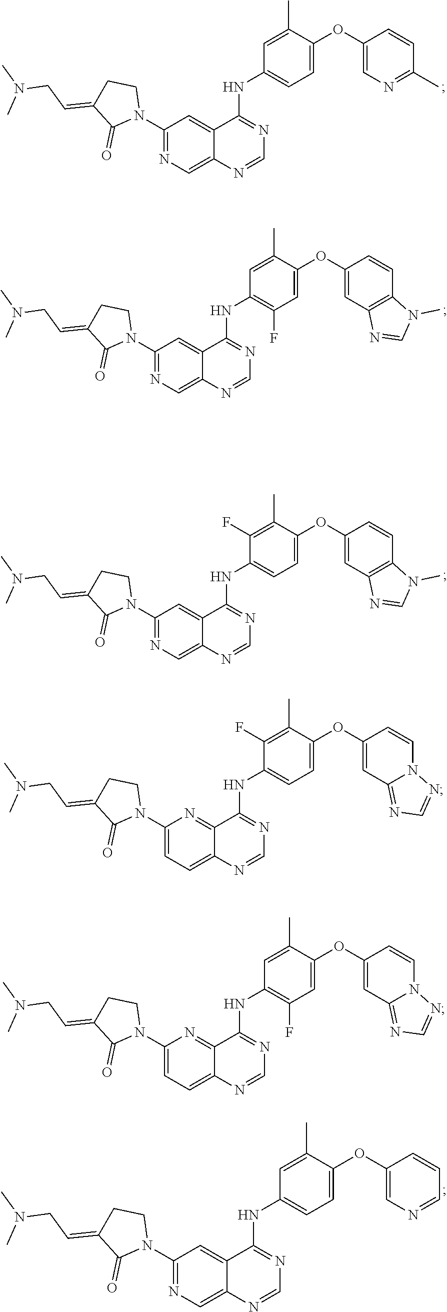 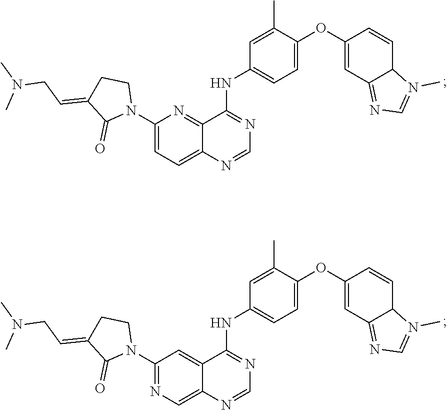 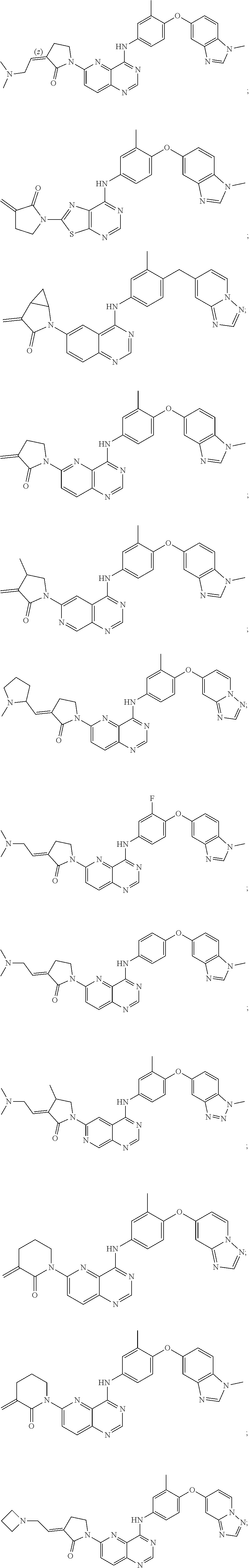 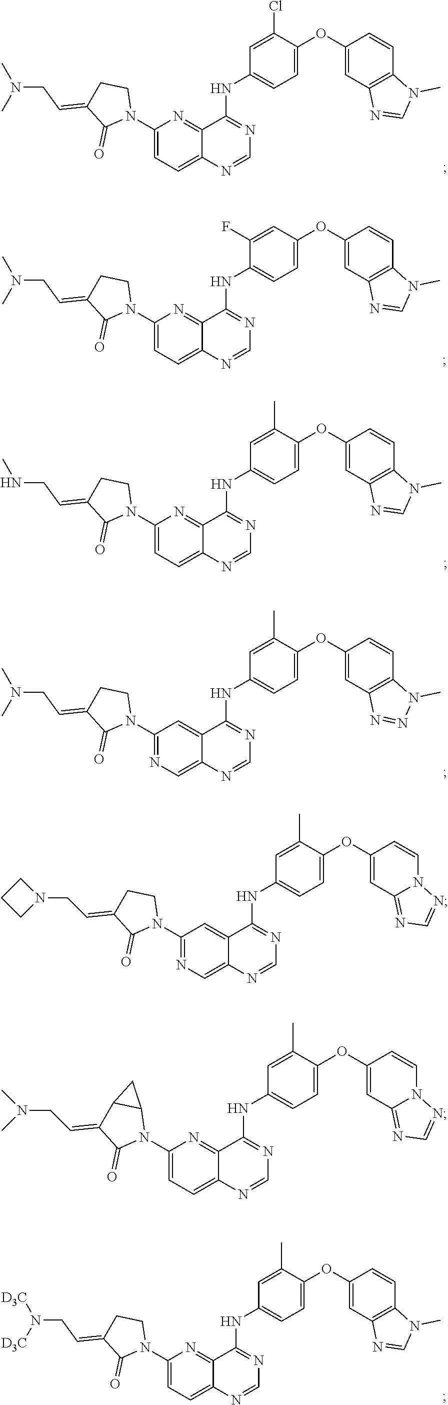 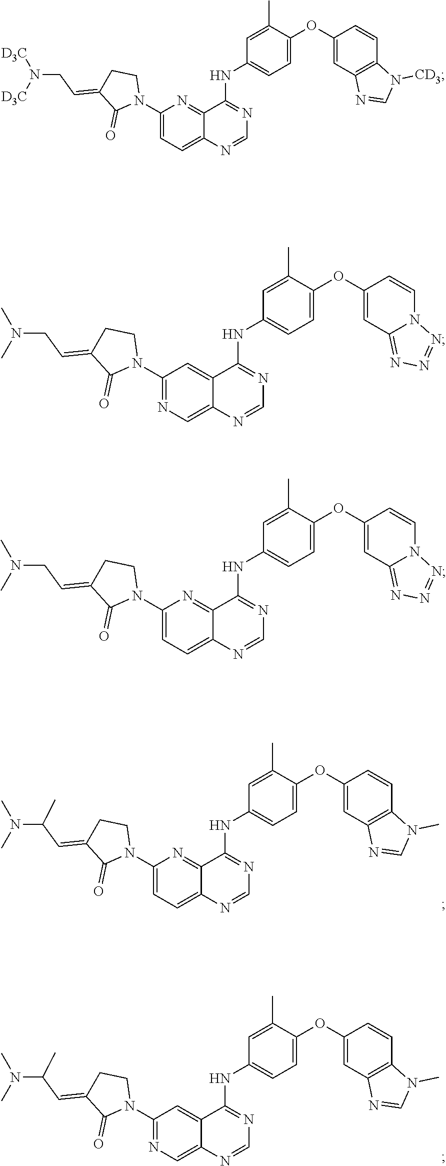 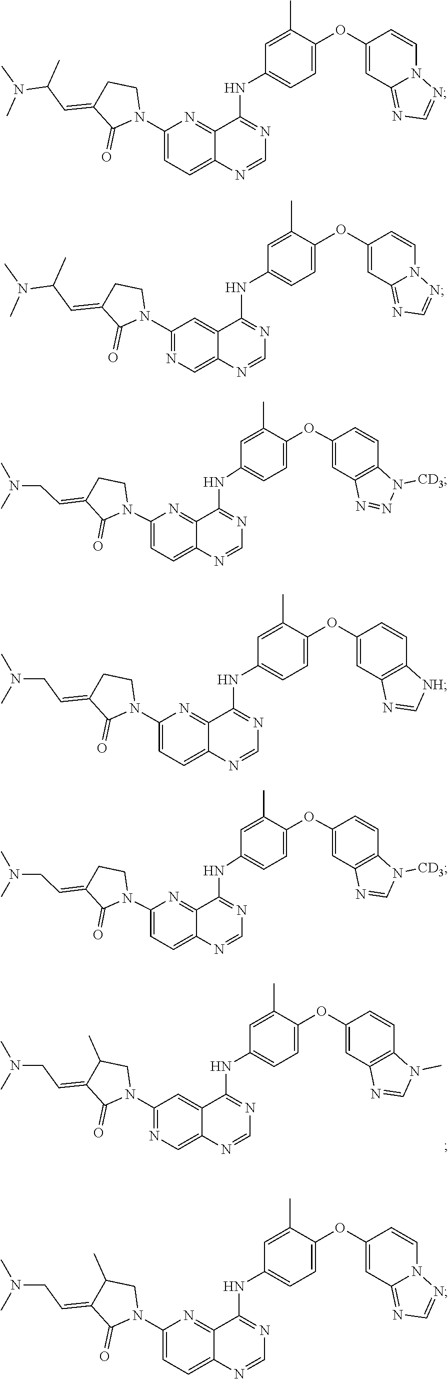 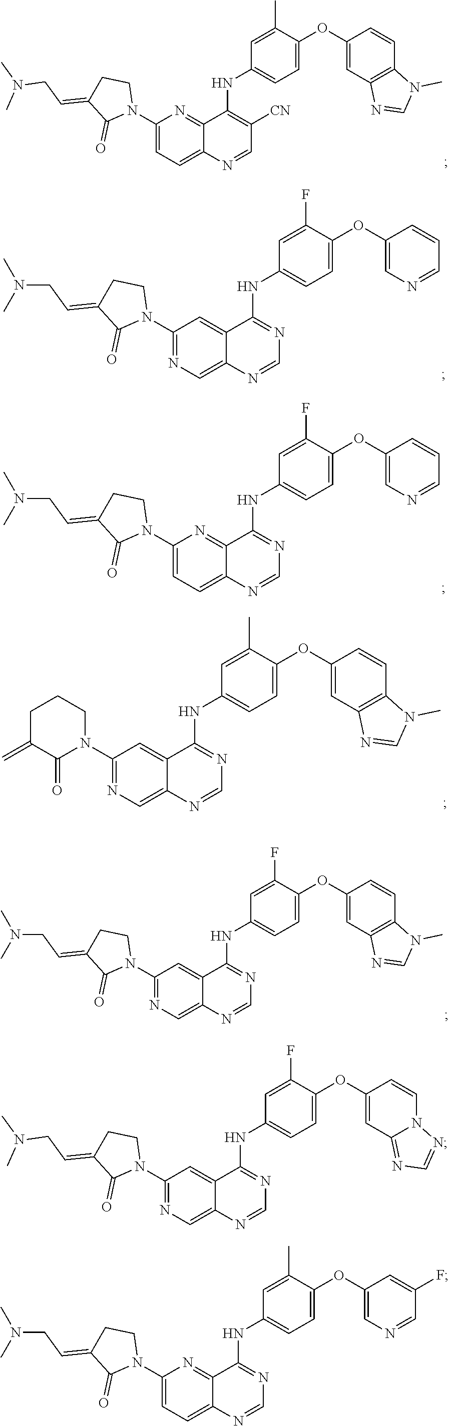 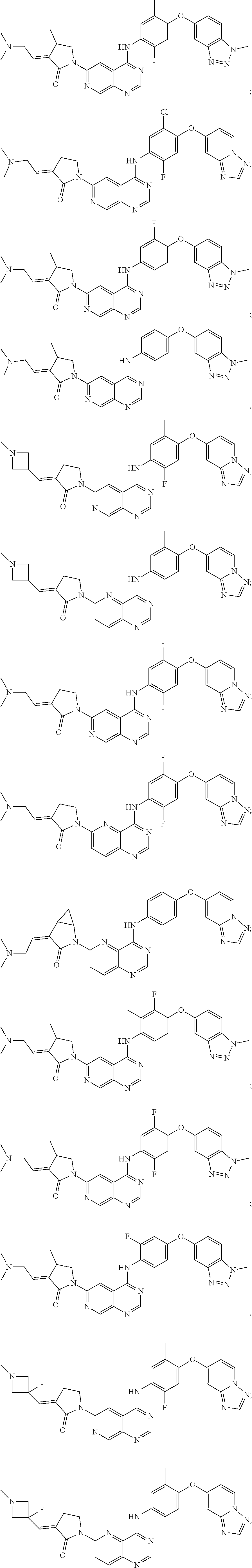 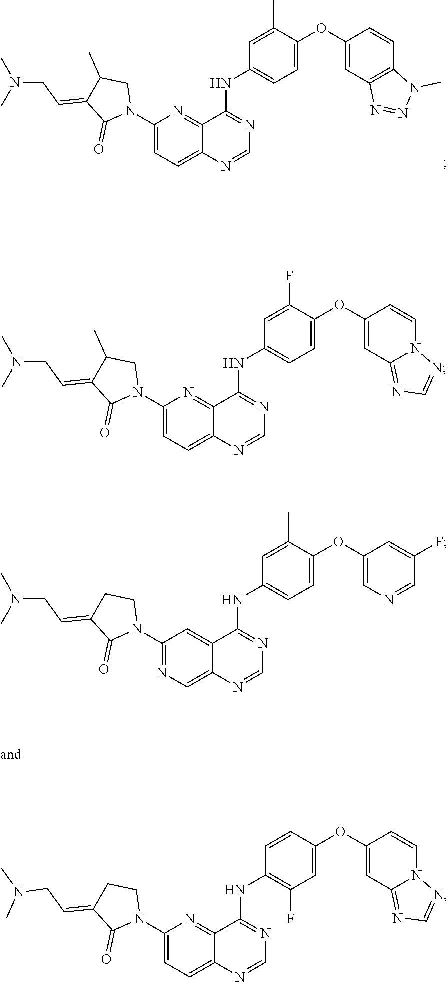 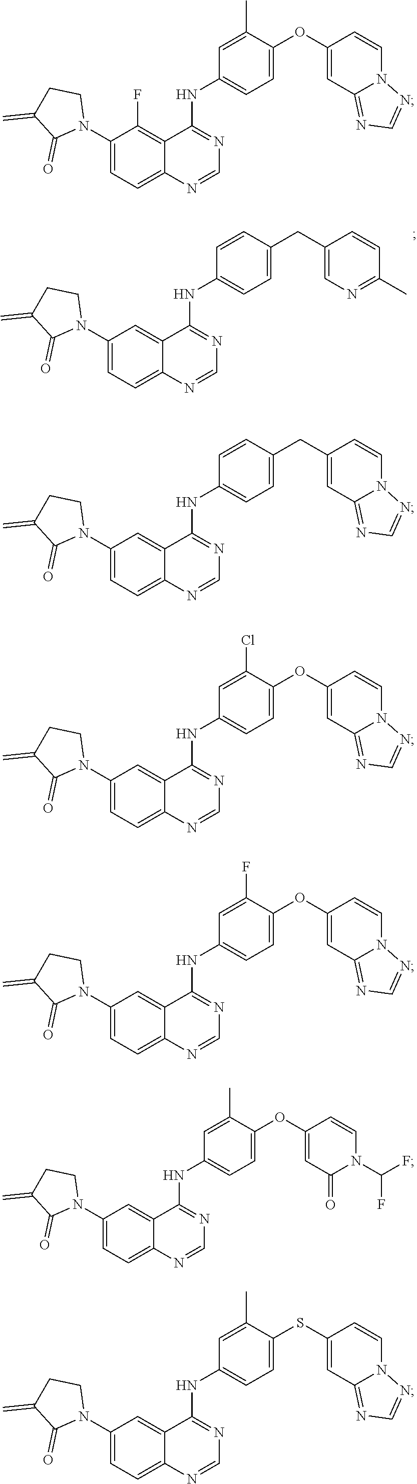 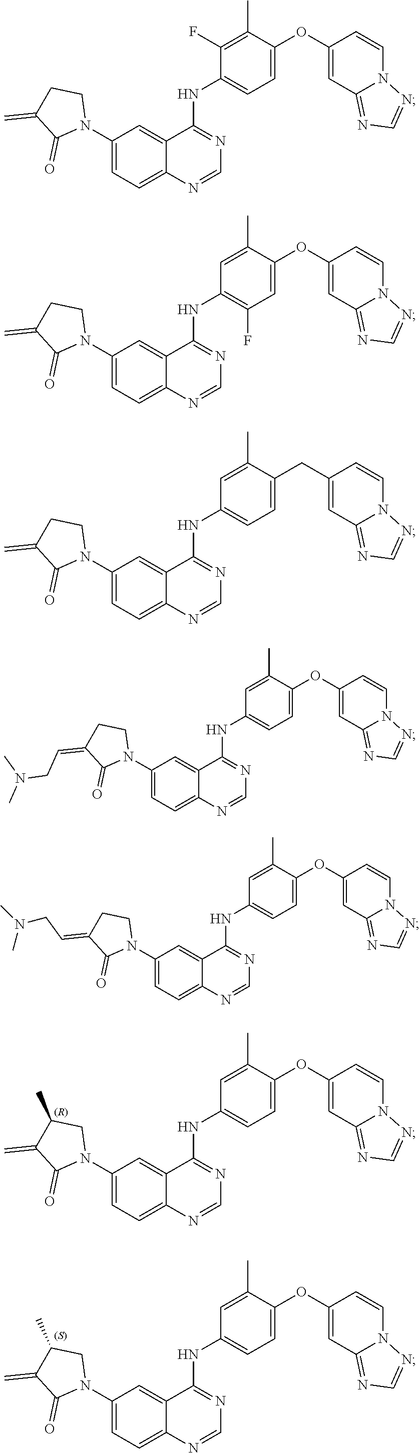 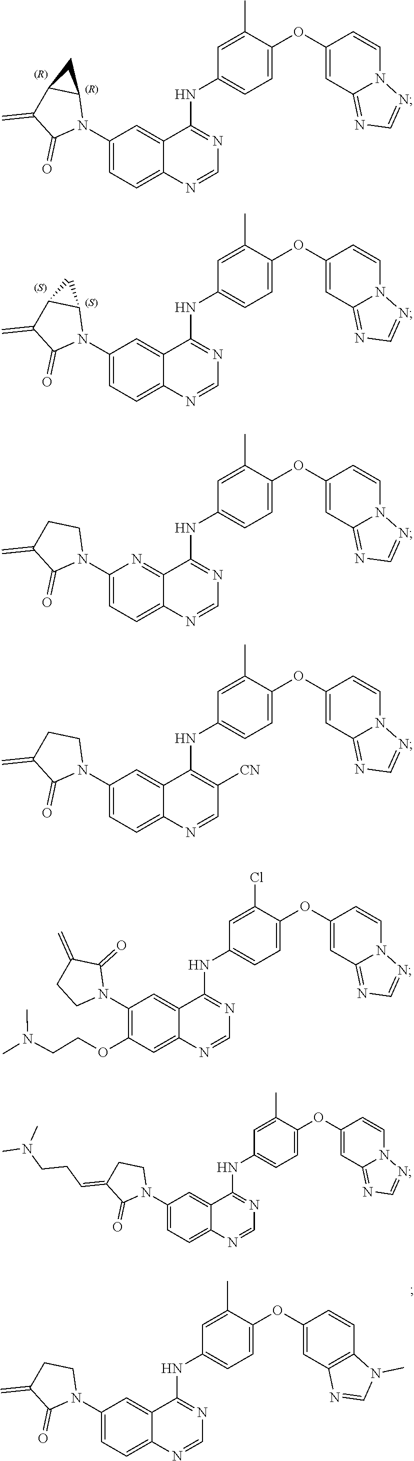 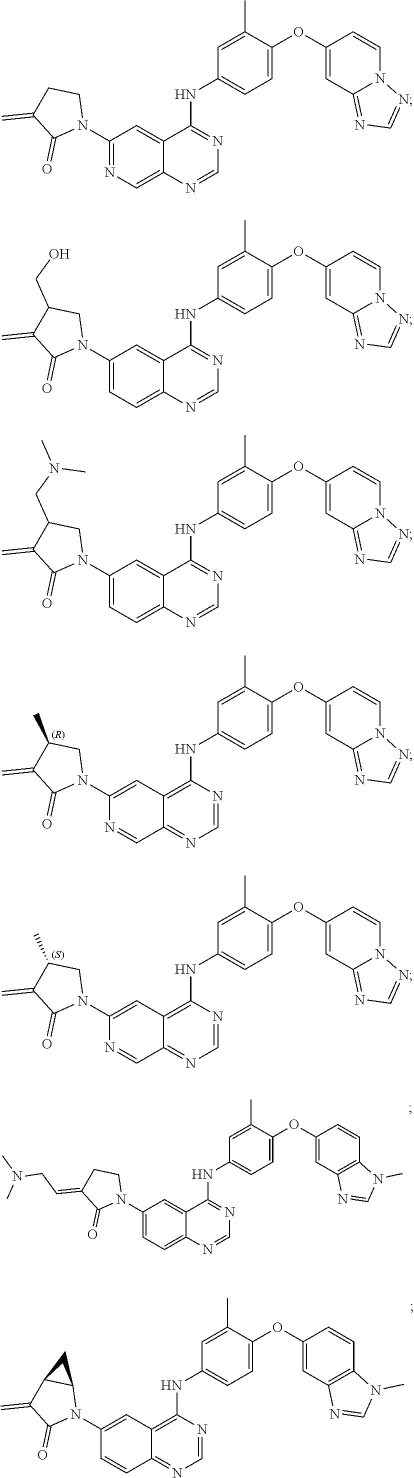 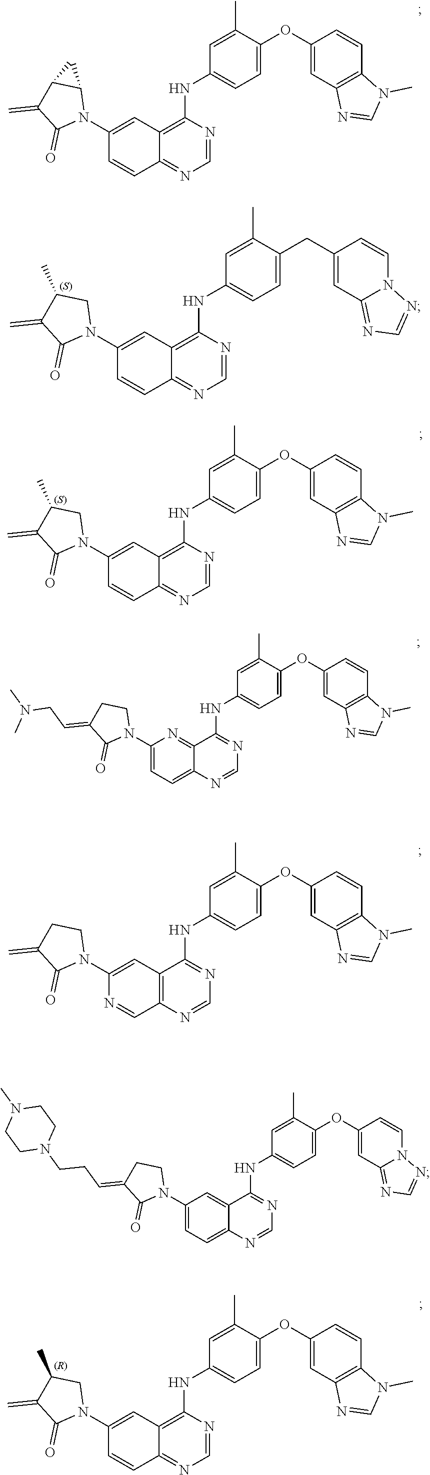 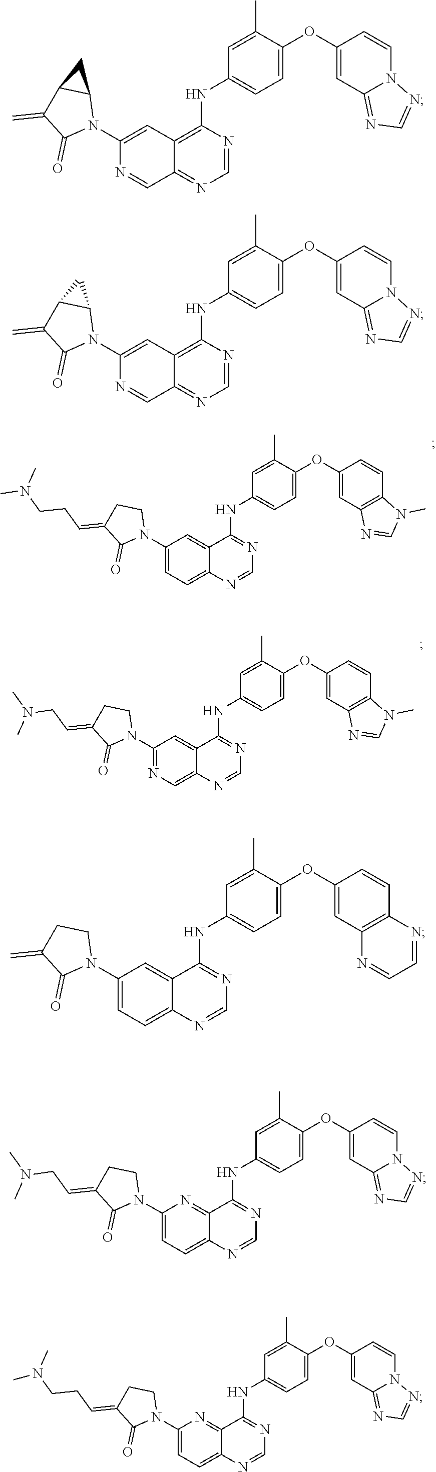 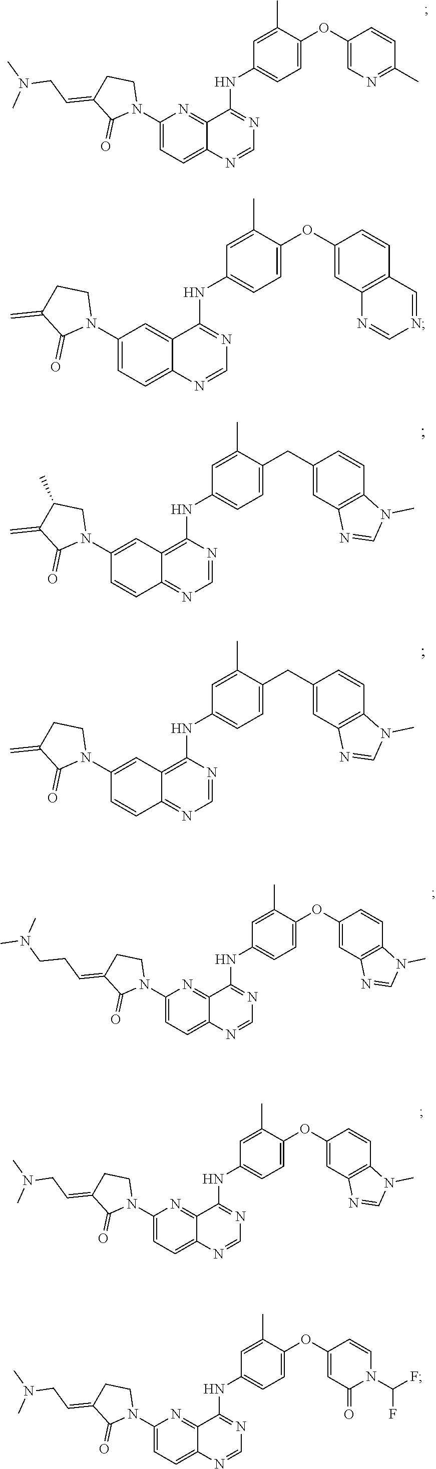 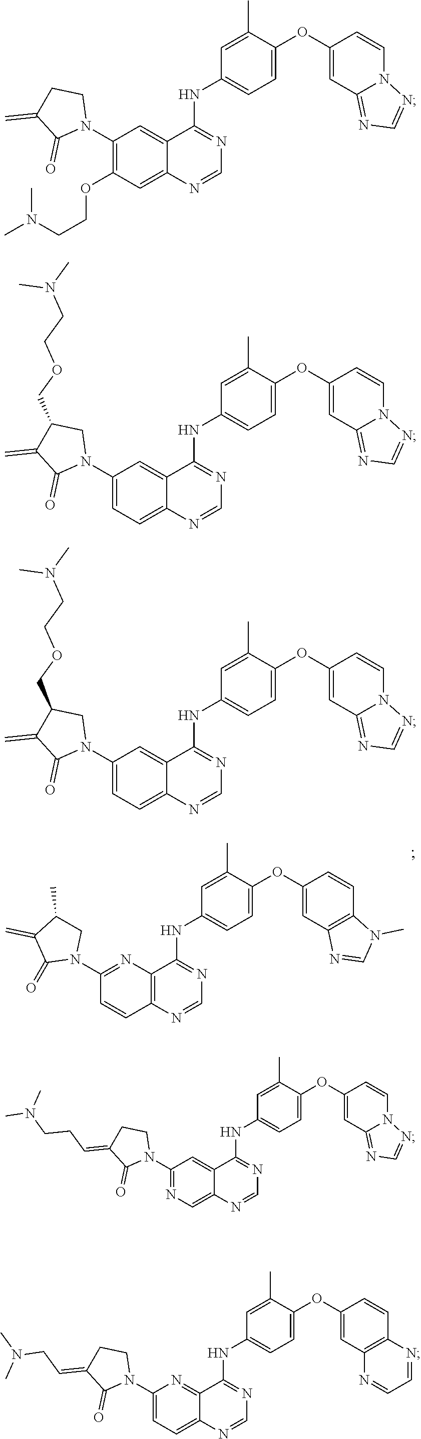 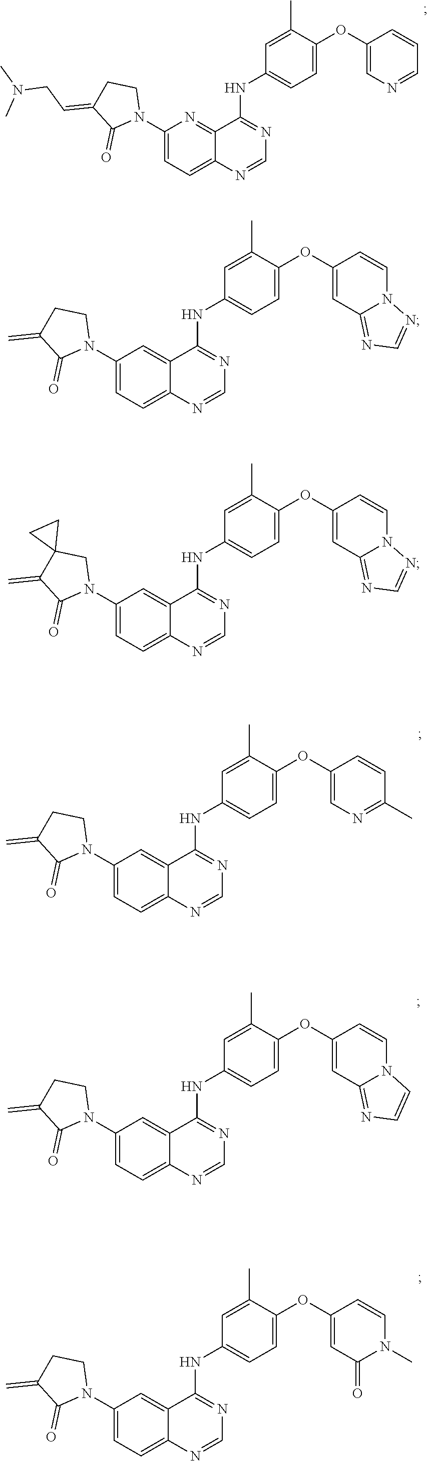 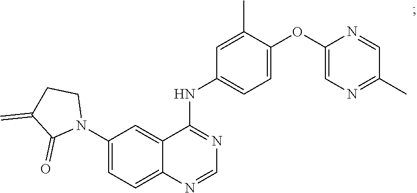 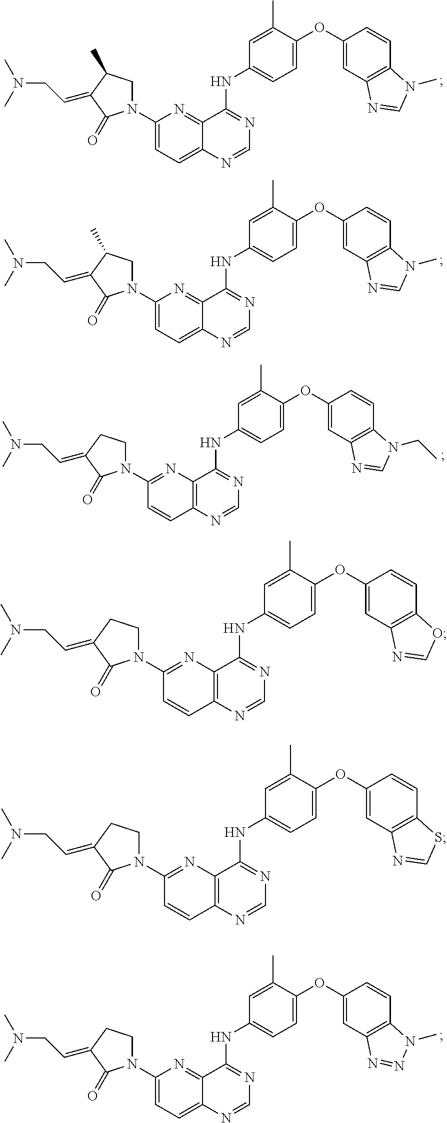 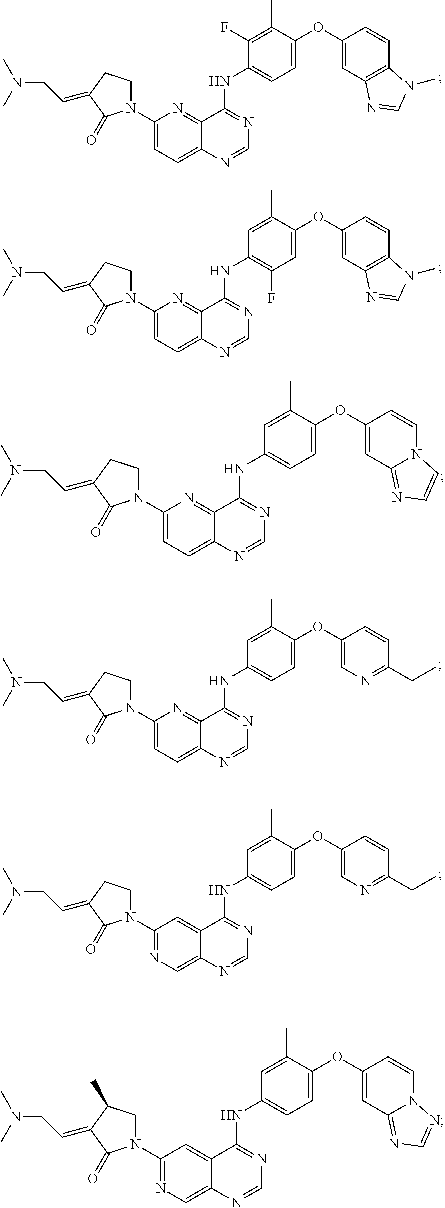 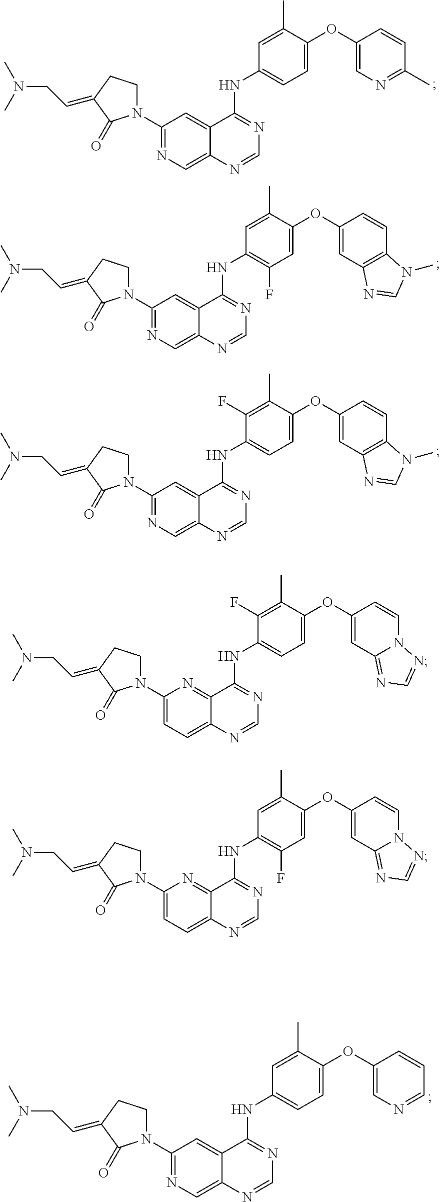 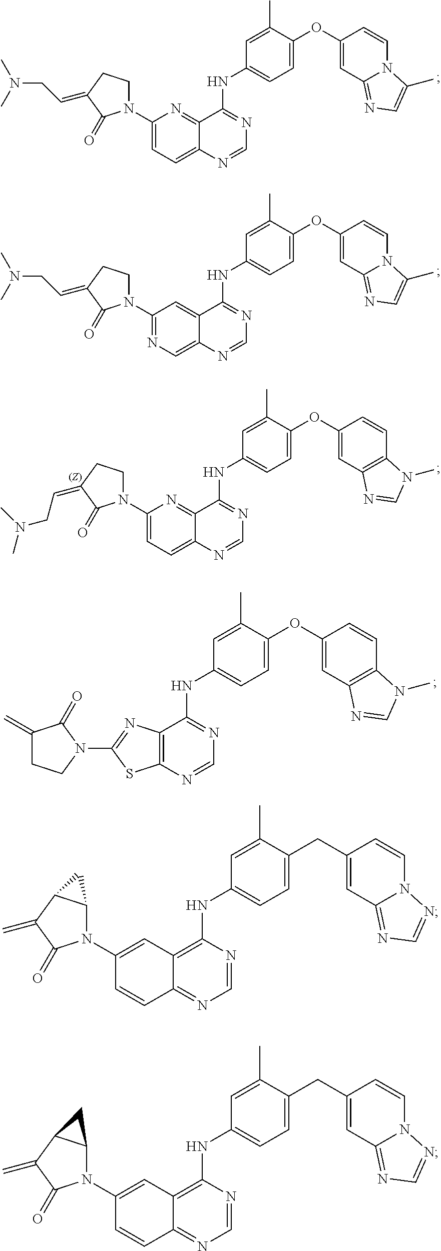 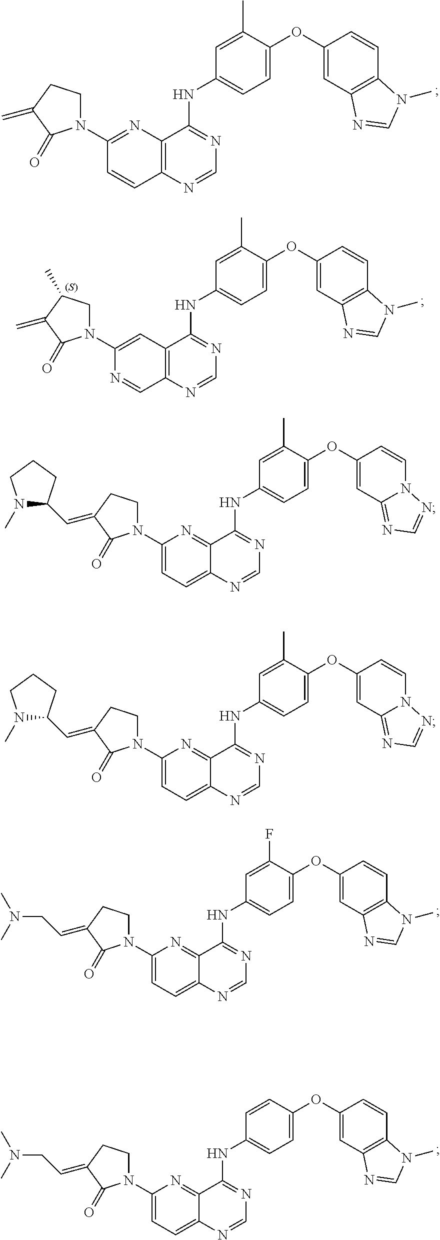 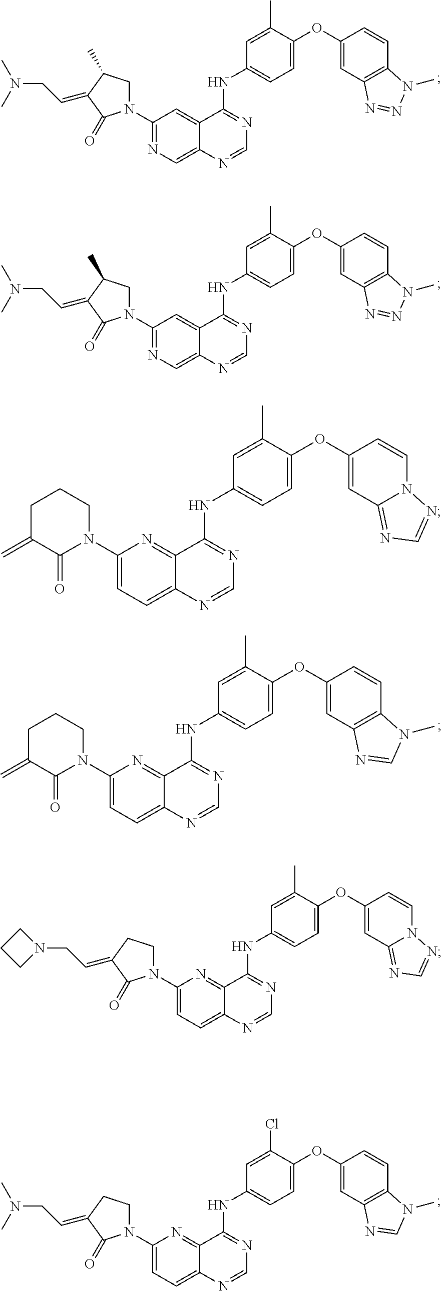 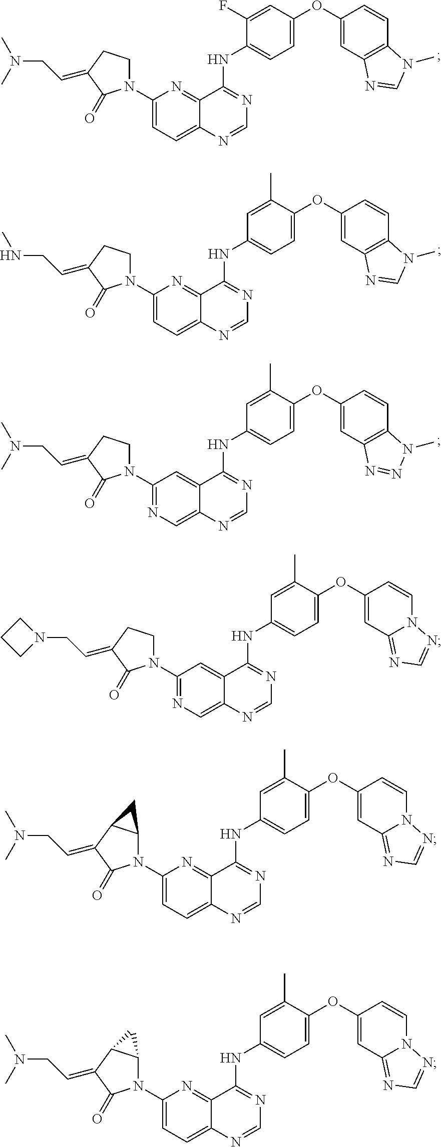 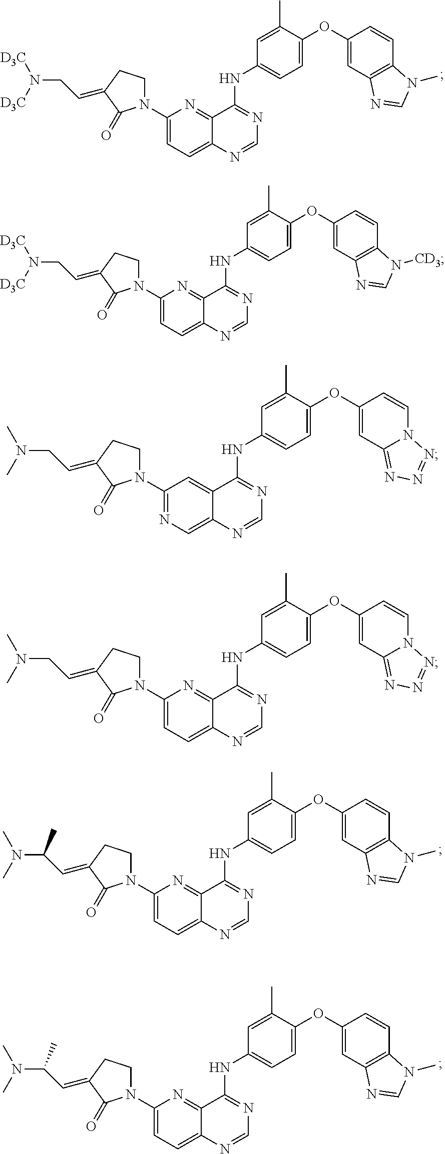 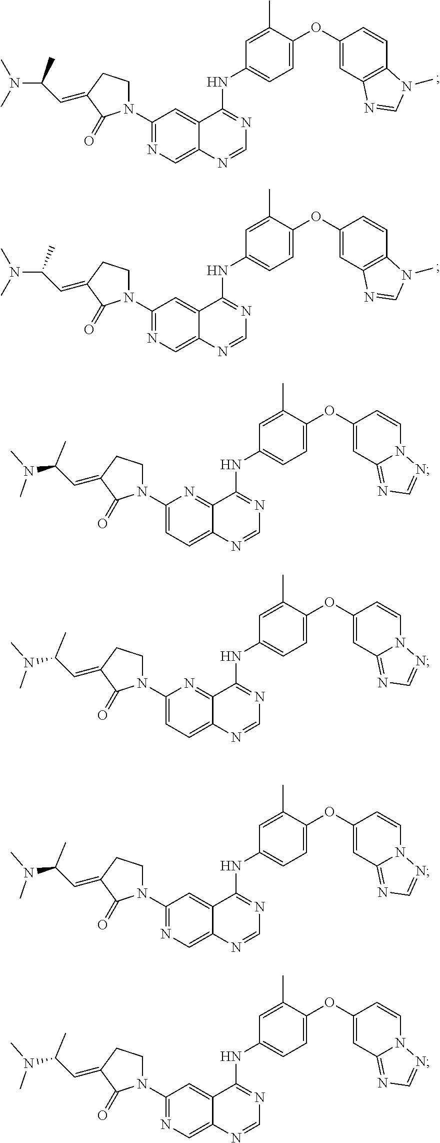 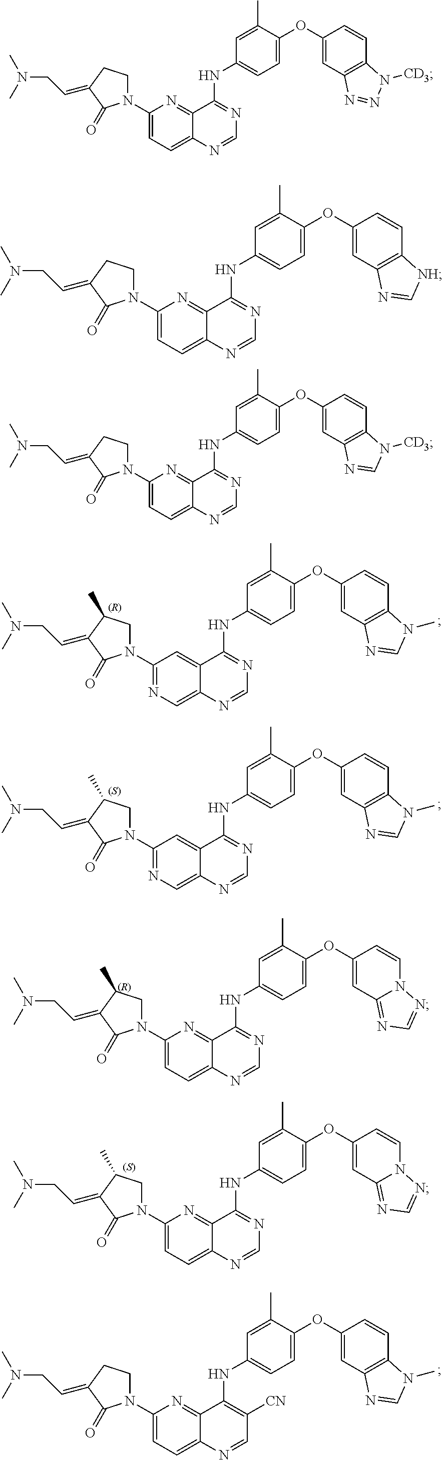 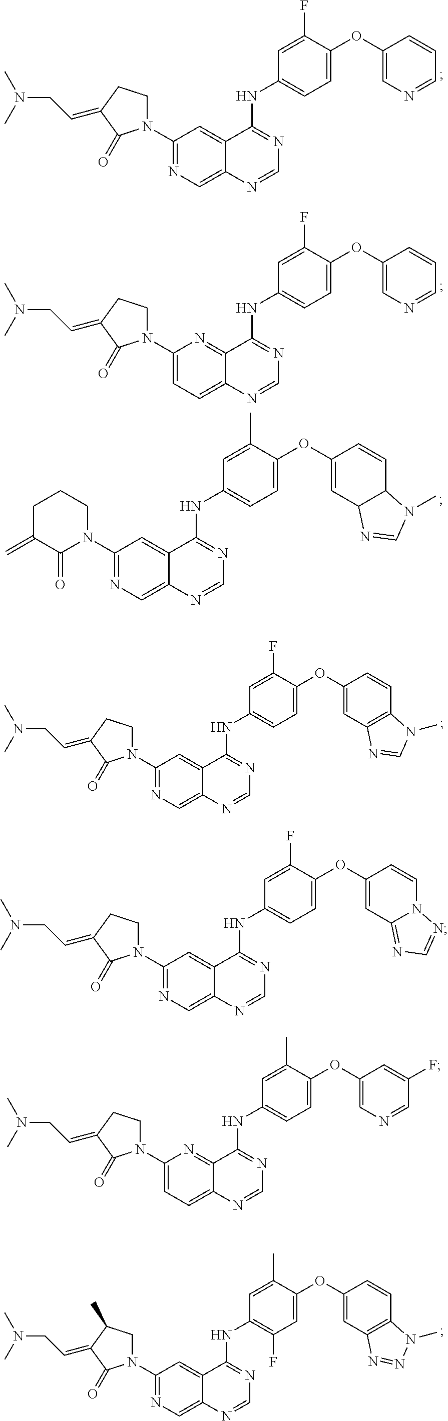 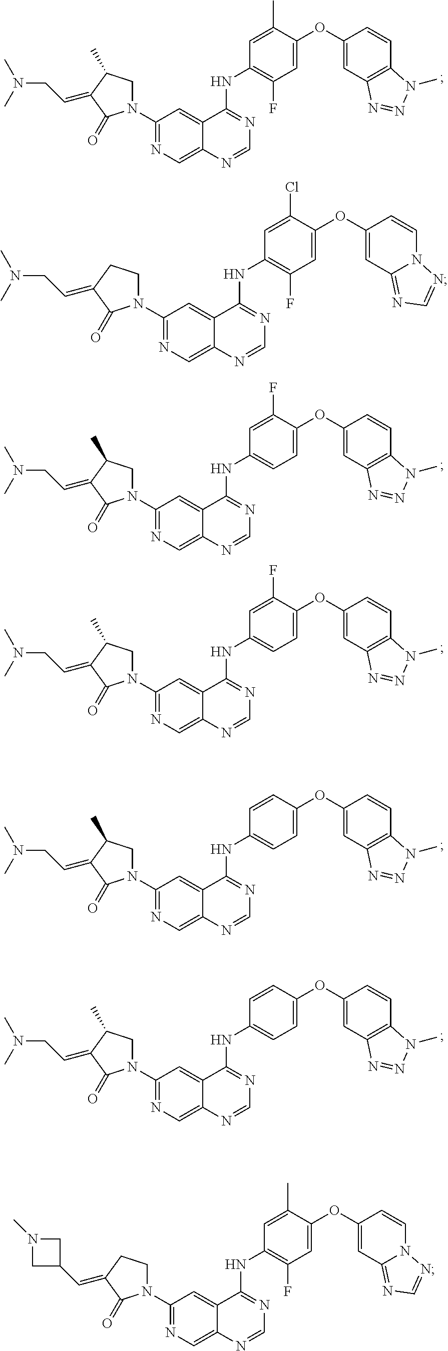 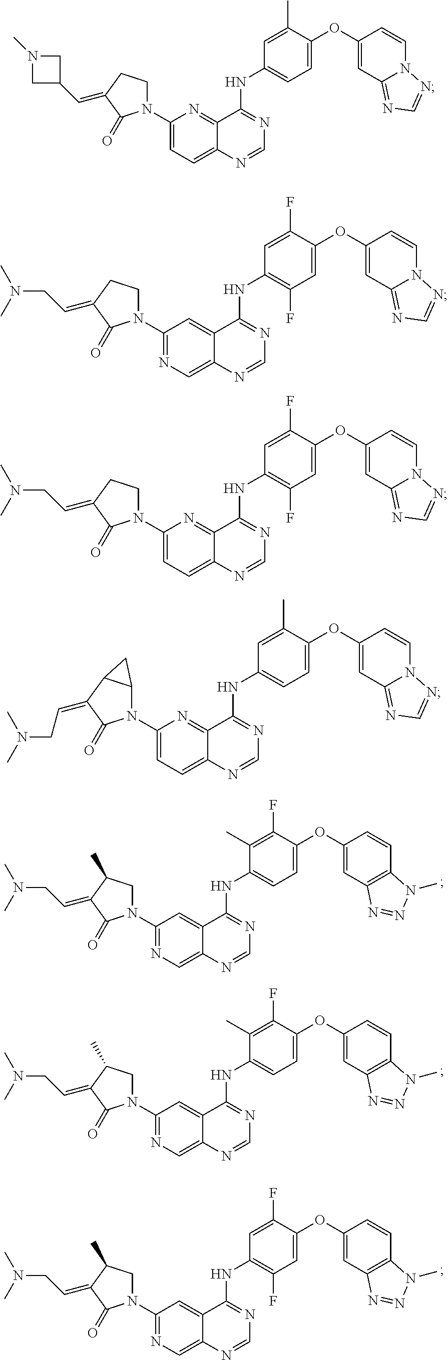 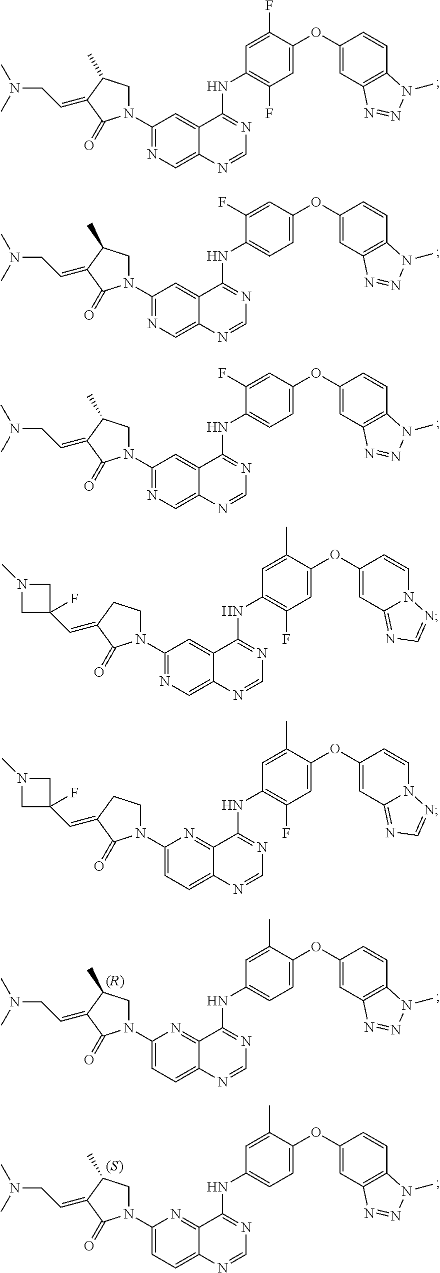 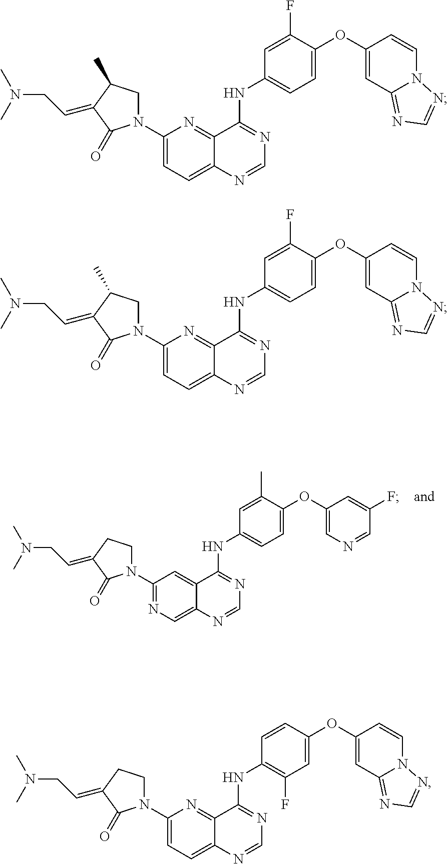 [SMILES: C[C@H]1CN(c2ccc3ncnc(Nc4ccc(Oc5ccn6ncnc6c5)c(F)c4)c3n2)C(=O)/C1=C/CN(C)C ; C[C@@H]1CN(c2ccc3ncnc(Nc4ccc(Oc5ccn6ncnc6c5)c(F)c4)c3n2)C(=O)/C1=C/CN(C)C ; Cc1cc(Nc2ncnc3cnc(N4CC/C(=C\CN(C)C)C4=O)cc23)ccc1Oc1cncc(F)c1 ; "and" ; CN(C)C/C=C1\CCN(c2ccc3ncnc(Nc4ccc(Oc5ccn6ncnc6c5)cc4F)c3n2)C1=O]
or a pharmaceutically acceptable salt, solvate, hydrate, or co-crystal thereof, or a mixture of any of the foregoing.

55. A compound selected from the group consisting of:
or a pharmaceutically acceptable salt, solvate, hydrate, or co-crystal thereof, or a mixture of any of the foregoing.

56. A pharmaceutical composition comprising the compound of any one of claims 1-55, or a pharmaceutically acceptable salt, solvate, hydrate, or co-crystal thereof, or a mixture of any of the foregoing, and at least one pharmaceutically acceptable excipient.

57. A method of inhibiting kinase activity of a human receptor tyrosine kinase ErbB2 or a mutant form of human ErbB2 comprising contacting the ErbB2 or the mutant form with a therapeutically effective amount of the compound of any one of claims 1-55, or a pharmaceutically acceptable salt, solvate, hydrate, or co-crystal thereof, or a mixture of any of the foregoing, or a therapeutically effective amount of the pharmaceutical composition of claim 56.

58. The method of claim 57, wherein the mutant form of human ErbB2 comprises a mutation in Exon 20.

59. The method of claim 57 or 58, wherein the mutant form of human ErbB2 comprises one or more mutations that introduce amino acid deletions and/or insertions selected from the group consisting of: A775_A776insYVMA, G778_P780insGSP, G776delinsVC, P780_Y781insGSP, M774delinsWLV, A775_G776insSVMA, A775_G776insI, G776delinsLC, G778_S779InsCPG, and V777_G778insGSP.

60. The method of claim 57, wherein the mutant form of human ErbB2 comprises a disease-associated point mutation in ErbB2.

61. The method of claim 57 or 60, wherein the mutant form of human ErbB2 comprises one or more point mutations in ErbB2 that introduce:
(a) an amino acid substitution selected from the group consisting of P122L, R217C, I263T, A293T, S305C, S310F/Y, H470Q, I655V, V659E, G660D, R678Q/C, L755R/S/P, I767M, D769H/N/Y, V777L/M, V8421, R868W, H878Y, E930K/D, E1021Q, F1030C, V11281, and N1219S; or
(b) a frameshift at A1232.

62. A method of treating a patient having a cancer, comprising administering to the patient a therapeutically effective amount of the compound of any one of claims 1-55, or a pharmaceutically acceptable salt, solvate, hydrate, or co-crystal thereof, or a mixture of any of the foregoing, or a therapeutically effective amount of the pharmaceutical composition of claim 56.

63. The method of claim 62, wherein the cancer comprises cells or cell tissue having increased ErbB2 kinase activity as compared to a control.

64. The method of claim 62 or 63, wherein the cancer comprises cells or cell tissue having one or more mutations in Exon 20 of the ErbB2.

65. The method of any one of claims 62-64, wherein the cancer comprises cells or cell tissue having one or more mutations in Exon 20 of the ErbB2 that introduce amino acid deletions and/or insertions selected from the group consisting of A775_A776insYVMA, G778_P780insGSP, G776delinsVC, P780_Y781insGSP, M774delinsWLV, A775_G776insSVMA, A775_G776insI, G776delinsLC, G778_S779InsCPG, and V777_G778insGSP.

66. The method of claim 62 or claim 63, wherein the cancer comprises cells or cell tissue having one or more disease-associated point mutations in ErbB2.

67. The method of any one of claims 62, 63 and 66, wherein the cancer comprises cells or cell tissue having one or more point mutations that introduce:
(a) an amino acid substitution selected from the group consisting of P122L, R217C, I263T, A293T, S305C, S310F/Y, H470Q, I655V, V659E, G660D, R678Q/C, L755R/S/P, I767M, D769H/N/Y, V777L/M, V8421, R868W, H878Y, E930K/D, E1021Q, F1030C, V11281, and N1219S; or
(b) a frameshift at A1232.

68. The method of any one of claims 62-67, wherein the cancer is lung, glioma, skin, head and neck, salivary gland, breast, esophageal, liver, stomach (gastric), uterine, cervical, biliary tract, pancreatic, colorectal, renal, bladder or prostate cancer.

69. The method of any one of claims 62-68, wherein the cancer is non-small cell lung cancer.

70. The method of any one of claims 62-69, wherein the patient has received at least one, at least two, or at least three prior therapies for the cancer.

71. The method of claim 70, wherein one or more of the prior therapies selected from the group consisting of lapatinib, neratinib, afatinib, pyrotinib, poziotinib, TAK-788, and tucatinib.

72. The method of any one of claims 62-71, wherein the method further comprises administering one or more additional anti-cancer agents.
